(19) United States
(12) Patent Application Publication
(10) Pub. No.: US 2025/0360194 A1
(43) Pub. Date: Nov. 27, 2025

(54) HERPES SIMPLEX VIRUS MRNA VACCINES

(71) Applicant: ModernaTX, Inc., Cambridge, MA (US)

(72) Inventors: Kapil Bahl, Medford, MA (US); Andrea Carfi, Cambridge, MA (US); Sumana Chandramouli, Cambridge, MA (US); Anthony DiPiazza, Cambridge, MA (US); Alec Freyn, Cambridge, MA (US); Kai Wu, Cambridge, MA (US); Yen-Ting Lai, Cambridge, MA (US)

(73) Assignee: ModernaTX, Inc., Cambridge, MA (US)

(21) Appl. No.: 18/717,792

(22) PCT Filed: Dec. 7, 2022
(86) PCT No.: PCT/US2022/081086

§ 371 Date: Jun. 7, 2024

Related U.S. Application Data
(60) Provisional application No. 63/287,208, filed on Dec. 8, 2021.
(60) Provisional application No. 63/298,112, filed on Jan. 10, 2022.

(51) Int. Cl. A61K 39/12; A61K 39/00; A61P 31/22; C07K 14/005

(52) U.S. Cl. CPC A61K 39/12; A61P 31/22; C07K 14/005; A61K 2039/53; A61K 2039/57; A61K 2039/70

(57) ABSTRACT

Provided herein are messenger ribonucleic acid (mRNA) vaccines encoding multiple herpes simplex virus (HSV) antigens involved in viral attachment and entry. Also provided are methods of using the vaccines and compositions comprising the vaccines.

### RELATED APPLICATIONS

This application claims the benefit under 35 U.S.C. § 119(e) of U.S. provisional application No. 63/287,208, filed Dec. 8, 2021, and U.S. provisional application No. 63/298,112, filed Jan. 10, 2022, each of which is incorporated by reference herein in its entirety.

### REFERENCE TO AN ELECTRONIC SEQUENCE LISTING

The contents of the electronic Sequence Listing (M137870195WO00-SEQ-NTJ.xml; Size: 214,658 bytes; and Date of Creation: Dec. 7, 2022) are herein incorporated by reference in their entirety.

### BACKGROUND

Herpes simplex viruses (HSV), belonging to one of two subtypes (HSV-1 or HSV-2), are double-stranded linear DNA viruses in the Herpesviridae family. These neuroinvasive viruses establish latent infections in nerve ganglia, with sporadic episodes of reactivation and replication causing recurrent symptomatic periods known as “outbreaks.” During such outbreaks, replication-competent virus particles are abundant in the affected area, and contact with the affected area allows for HSV transmission. HSV affects many people worldwide. The World Health Organization estimates that 67 percent of people under the age of 50 are infected with HSV-1 while 11 percent of people between the ages of 15 and 49 are infected with HSV-2.

Currently, antiviral drugs, including acyclovir (Zovirax®), famciclovir (Famvir®), and valacyclovir (Valtrex®), are the only treatments approved by the FDA that people can take to fight HSV. Herpes viruses are more complicated and more evasive than most viruses, so developing a vaccine has been challenging. In fact, several companies that were overseeing clinical trials on a herpes vaccine over the past few years have since abandoned their research. For example, in June of 2018, one company announced the phase II clinical trial for its HSV-2 vaccine did not meet “its primary endpoint.” In September of 2017, another company announced it was exploring “strategic alternatives” for its herpes vaccine but ultimately ceased spending on the vaccine.

Symptomatic infections by other Herpesviridae are often prevented with the use of live-attenuated vaccines, such as attenuated varicella-zoster virus (VZV) for preventing chickenpox and shingles. While clinical trials using live-attenuated HSV vaccines are ongoing, there remains an associated risk of the live virus establishing a latent infection in a vaccinated subject, predisposing the subject to outbreaks later in life. Thus, there is still an urgent need to develop safe and effective HSV vaccines.

### SUMMARY

The messenger ribonucleic acid (mRNA) vaccines provided herein safely direct the body's cellular machinery to produce multiple modified HSV proteins designed to have therapeutically immunogenic activity inside and outside of cells. These HSV mRNA vaccines comprise multiple mRNA polynucleotides, each of which encodes a different intracellular or cell-surface expressed protein strategically designed to elicit improved, balanced humoral and cellular immune responses against HSV. Additionally, the intracellular HSV antigens are designed to prevent deleterious effects of HSV protein expression. Modifications to the surface-expressed HSV glycoproteins improve expression and the antibody response, while modifications to the HSV intracellular proteins elicit an improved CD8⁺ T cell response that can clear cells in which the HSV has re-emerged from latency or is actively replicating and expressing the wild-type protein counterparts. Surprisingly, the modified HSV intracellular proteins produced following mRNA vaccination inhibit the generation of pathogenic glycoprotein-specific Th2 cells without compromising the generation of immunoprotective glycoprotein-specific Th1 cells or glycoprotein-specific antibodies.

HSV mRNA vaccination, in some aspects, results in the expression of HSV glycoprotein B (gB), HSV glycoprotein C (gC), and/or HSV glycoprotein D (gD) by cells of the body, in a similar manner to when the proteins are expressed by the native virus, eliciting the production of antibodies and T cells specific to the glycoproteins (e.g., Th1 cells that produce pro-inflammatory IFN-γ and CD8+ T cells that clear infected cells). Because each of HSV glycoproteins B, C, and D are required for viral entry, multivalent mRNA vaccines against these antigens generate potent neutralizing antibody responses that limit (e.g., prevent) and/or treat HSV infection. Modifications to these antigens are shown herein to improve the antibody response. For example, deletion of the cytoplasmic tail of HSV gC elicits higher antibody titers relative to the wild-type (unmodified) form. As another example, mutation of residue 327 (e.g., F327A) of HSV gC abrogates binding and sequestration of human C3b, which exposes gC epitopes important for immunization that would otherwise be masked by C3b.

Antibodies generated in response to the HSV mRNA vaccines provided herein have multiple antiviral activities that are useful in preventing or ameliorating HSV infections. For example, neutralizing activity towards HSV particles, preventing cellular infection in microneutralization assays. Additionally, elicited antibodies promote antibody-dependent cell-mediated cytotoxicity, which causes the clearance of virally-infected cells. Furthermore, antibodies prevent cell-cell spread by HSV particles, in which HSV released from one cell translocate and infect neighboring cells. Therefore, in addition to their prophylactic uses in preventing HSV infection in naïve or recently exposed subjects, the vaccines of the present disclosure are also useful therapeutically, such as for reducing the duration of an HSV outbreak or preventing reactivation of latent HSV infection.

HSV mRNA vaccination, in other aspects, also results in the expression of HSV intracellular proteins ICP0 and/or ICP4, which are expressed inside the host cell early during reactivation of latent infection. These intracellular proteins, in some embodiments, have been modified to prevent the deleterious effects of HSV protein expression and are capable of eliciting a CD8⁺ T cell response than can clear cells in which HSV has re-emerged from latency or is actively replicating, as discussed above. Modifications to the intracellular HSV proteins include, for example, internal truncations to (i) remove regions of the proteins that are sparse in known CD8⁺ T cell epitopes, thereby increasing the epitope density of modified proteins (e.g., ICP0 and/or ICP4), and/or (ii) disrupt or remove functional portions of the proteins to improve safety or immunogenicity. In some embodiments, an intracellular HSV protein is modified with a disrupted nuclear localization signal, promoting retention in the cytosol, proteasomal processing, and epitope presentation to CD8+ T cells. Additionally, while wild-type ICP0 and ICP4, for example, inhibit innate immune function to facilitate viral replication (e.g., via a USP7-binding domain involved in inhibiting Toll-like receptor signaling, or a RING finger domain involved in inhibiting antiviral interferon responses), these domains may be disrupted to reduce or eliminate these immunosuppressive functions, while retaining immunogenic CD8⁺ T cell epitopes.

Thus, compositions containing mRNAs that collectively encode HSV gB, gC, gD, ICP0, and ICP4 are useful for generating glycoprotein-specific antibodies and robust antiviral Th1 cell responses that control viral replication, while limiting the generation of pathogenic Th2 cells that exacerbate HSV-2 infection.

Some aspects relate to a herpes simplex virus (HSV) messenger ribonucleic acid (mRNA) vaccine comprising (a) an mRNA comprising an open reading frame encoding an HSV glycoprotein B (gB) that comprises a truncated C-terminus, relative to a wild-type HSV gB; (b) an mRNA comprising an open reading frame encoding an HSV glycoprotein C (gC); (c) an mRNA comprising an open reading frame encoding an HSV glycoprotein D (gD); (d) an mRNA comprising an open reading frame encoding an HSV intracellular protein 0 (ICP0); (e) an mRNA comprising an open reading frame encoding an HSV intracellular protein 4 (ICP4); and (f) a lipid nanoparticle.

In some embodiments, the HSV gB comprises a truncated cytoplasmic tail. In some embodiments, the HSV gB does not comprise a cytoplasmic tail.

In some embodiments, the HSV gC comprises an F327A substitution and a truncated C-terminus, relative to a wild-type HSV gC. In some embodiments, the HSV gC comprises a truncated cytoplasmic tail. In some embodiments, the HSV gB and/or HSV gC does not comprise a cytoplasmic tail.

In some embodiments, the HSV ICP0 lacks a nuclear localization signal and/or a RING finger domain.

In some embodiments, the HSV ICP0 comprises a higher density of CD8+ T cell epitopes, relative to a wild-type HSV ICP0.

In some embodiments, the HSV ICP4 lacks a nuclear localization signal and/or a RING finger domain.

In some embodiments, the HSV ICP4 comprises a higher density of CD8+ T cell epitopes, relative to a wild-type HSV ICP4.

Other aspects relate to a herpes simplex virus (HSV) messenger ribonucleic acid (mRNA) vaccine comprising (a) an mRNA comprising an open reading frame encoding an HSV glycoprotein B (gB) that comprises a truncated C-terminus, relative to a wild-type HSV gB; (b) an mRNA comprising an open reading frame encoding an HSV glycoprotein C (gC) that comprises a truncated C-terminus, relative to a wild-type HSV gC; (c) an mRNA comprising an open reading frame encoding an HSV glycoprotein D (gD); (d) an mRNA comprising an open reading frame encoding an HSV intracellular protein 0 (ICP0) that comprises a higher density of CD8+ T cell epitopes relative to a wild-type HSV ICP0; (e) an mRNA comprising an open reading frame encoding an HSV intracellular protein 4 (ICP4) that comprises a higher density of CD8+ T cell epitopes relative to a wild-type HSV ICP4; and (f) a lipid nanoparticle.

Further aspects relate to a herpes simplex virus (HSV) messenger ribonucleic acid (mRNA) vaccine comprising (a) an mRNA comprising an open reading frame encoding an HSV glycoprotein B (gB), optionally wherein the gB comprises a truncated C-terminus, relative to a wild-type HSV gB; (b) an mRNA comprising an open reading frame encoding a HSV glycoprotein C (gC) that comprises an F327A substitution, and a truncated C-terminus, relative to a wild-type HSV gC; and (c) an mRNA comprising an open reading frame encoding a wild-type HSV glycoprotein D (gD); (d) a lipid nanoparticle.

In some embodiments, the method further comprises an mRNA comprising an open reading frame encoding a HSV intracellular protein 0 (ICP0) comprising a higher density of CD8+ T cell epitopes relative to a wild-type HSV ICP0.

In some embodiments, the method further comprises an mRNA comprising an open reading frame encoding a HSV intracellular protein 4 (ICP4) comprising a higher density of CD8+ T cell epitopes relative to a wild-type HSV ICP4.

In some embodiments, the HSV gB and/or HSV gC comprises a truncated cytoplasmic tail. In some embodiments, the HSV gB and/or HSV gC does not comprise a cytoplasmic tail.

Still other aspects relate to a herpes simplex virus (HSV) messenger ribonucleic acid (mRNA) vaccine comprising (a) an mRNA comprising an open reading frame encoding a HSV glycoprotein C (gC) that comprises an F327A substitution, and a truncated C-terminus, relative to a wild-type HSV gC; (b) an mRNA comprising an open reading frame encoding a wild-type HSV glycoprotein D (gD); (c) an mRNA comprising an open reading frame encoding a HSV intracellular protein 0 (ICP0) comprising a higher density of CD8+ T cell epitopes relative to a wild-type HSV ICP0; (d) an mRNA comprising an open reading frame encoding a HSV intracellular protein 4 (ICP4) comprising a higher density of CD8+ T cell epitopes relative to a wild-type HSV ICP4; and (e) a lipid nanoparticle.

In some embodiments, the HSV gC comprises a truncated cytoplasmic tail.

In some embodiments, the HSV gC does not comprise a cytoplasmic tail.

In some embodiments, the vaccine induces a Th1-polarized CD4+ T cell-mediated immune response to the HSV gC, gB, and/or gD.

In some embodiments, the vaccine elicits more Th1 cells that are specific to an antigen selected from HSV gB, gC, or gD, than Th2 cells specific to the antigen.

In some embodiments, a population of CD4+ T cells specific to an antigen selected from HSV gB, gC, or gD, comprises more than 50% Th1 cells.

In some embodiments, each of the HSV gB, gC, and gD comprises a transmembrane domain.

In some embodiments: (a) the HSV gB has a length of about 798 amino acids; (b) the HSV gC has a length of about 469 amino acids; (c) the HSV ICP0 comprises a truncation in a nuclear localization signal, RING finger domain, and/or USP7-binding domain relative to a wild-type HSV ICP0; and/or (d) the HSV ICP4 comprises a truncation in a nuclear localization signal and/or DNA-binding domain relative to a wild-type HSV ICP4.

In some embodiments, the HSV ICP0 does not comprise a nuclear localization signal, does not comprise a USP7-binding domain, and/or does not comprise a RING finger domain.

In some embodiments, the HSV ICP0 does not comprise a nuclear localization signal and/or comprises a truncated DNA-binding domain.

In some embodiments: (a) the HSV gB comprises an amino acid sequence having at least 90%, at least 95%, or 100% identity to the amino acid sequence of SEQ ID NO: 54; (b) the HSV gC comprises an amino acid sequence having at least 90%, at least 95%, or 100% identity to the amino acid sequence of SEQ ID NO: 63; (c) the HSV gD comprises an amino acid sequence having at least 90%, at least 95%, or 100% identity to the amino acid sequence of SEQ ID NO: 39; (d) the HSV ICP0 comprises an amino acid sequence having at least 90%, at least 95%, or 100% identity to the amino acid sequence of SEQ ID NO: 47; and/or (e) the HSV ICP4 comprises an amino acid sequence having at least 90%, at least 95%, or 100% identity to the amino acid sequence of SEQ ID NO: 49.

In some embodiments, the molar ratio of mRNA of (c) and (d) to the mRNA of (a), (b), and (c) is no more than 0.8:1.

In some embodiments, the one or more mRNAs comprise a chemical modification.

In some embodiments, 100% of the uracil nucleotides of the one or more mRNAs comprise a chemical modification.

In some embodiments, the chemical modification is 1-methylpseudouracil.

In some embodiments, the lipid nanoparticle comprises an ionizable lipid, a neutral lipid, a sterol, and a PEG-modified lipid.

In some embodiments, the lipid nanoparticle comprises 40-50 mol % ionizable lipid, 5-15 mol % neutral lipid, 30-50 mol % sterol, and 0.5-3 mol % PEG-modified lipid.

In some embodiments: the ionizable lipid comprises a structure of Compound (I):

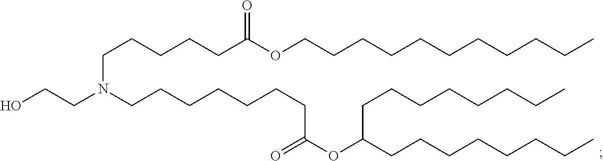 [SMILES: CCCCCCCCCCCOC(=O)CCCCCN(CCO)CCCCCCCC(=O)OC(CCCCCCCC)CCCCCCCC]

the neutral lipid is distearoylphosphatidylcholine (DSPC);
the sterol is cholesterol; and/or
the PEG-modified lipid is 1,2 dimyristoyl-sn-glycerol, methoxypolyethyleneglycol (PEG-DMG).

Some aspects relate to a method comprising administering to a subject the vaccine of any one of the preceding aspects or embodiments.

In some embodiments, the subject has an HSV infection or has been exposed to HSV.

In some embodiments, the vaccine is administered in an amount effective for preventing a latent HSV infection in the subject.

In some embodiments, the vaccine is administered in an amount effective for preventing reactivation of a latent HSV infection in the subject, for preventing replication of HSV, reducing duration of an HSV infection in the subject, for reducing a number of replication-competent HSV particles in the subject, and/or for reducing a number of cells in the subject that comprise an HSV genome.

In some embodiments, the vaccine induces a CD4+ T cell-mediated immune response to the HSV gB, gC, and/or gD, and the CD4+ T cells bind to one or more CD4+ T cell epitopes of the HSV gB, gC, or gD.

In some embodiments, at least 50% of the CD4+ T cells produce one or more cytokines selected from the group consisting of IFN-γ, IL-2, and TNF-α.

In some embodiments, fewer than 10% of the CD4+ T cells produce any one or more of IL-4, IL-5, IL-9, IL-10, or IL-13.

In some embodiments, the vaccine induces a CD8+ T cell-mediated immune response to the HSV ICP0 and/or or ICP4, and the CD8+ T cells bind to one or more CD8+ T cell epitopes of the HSV ICP0 and or ICP4.

In some embodiments, the CD8+ T cells are cytotoxic.

Other aspects relate to a modified herpes simplex virus (HSV) intracellular protein 0 (ICP0) comprising fewer amino acids than a wild-type HSV ICP0.

In some embodiments, the modified HSV ICP0 comprises a truncation in a nuclear localization signal relative to a wild-type HSV ICP0.

In some embodiments, the modified HSV ICP0 does not comprise a nuclear localization signal.

In some embodiments, the modified HSV ICP0 comprises a truncation in a RING finger domain relative to a wild-type HSV ICP0.

In some embodiments, the modified HSV ICP0 does not comprise a RING finger domain.

In some embodiments, the modified HSV ICP0 comprises a truncation in a USP7-binding domain relative to the wild-type HSV ICP0.

In some embodiments, the modified HSV ICP0 does not comprise a USP7-binding domain.

In some embodiments, the modified HSV ICP0 comprises a linker between a first portion of the modified HSV ICP0 and a second portion of the modified HSV ICP0.

In some embodiments, the linker comprises 2-10 glycine residues.

In some embodiments, the modified HSV ICP0 comprises an amino acid sequence having a length that is no more than 65% the length of the wild-type HSV IPC0.

In some embodiments, the modified HSV ICP0 amino acid sequence comprises at least 65% as many T cell epitopes as the wild-type HSV ICP0.

In some embodiments, the modified HSV ICP0 comprises about 487 amino acids.

Other aspects relate to ribonucleic acid (RNA) comprising an open reading frame encoding the modified HSV ICP0 of any one of the preceding aspects or embodiments.

Yet other aspects relate to a modified herpes simplex virus (HSV) intracellular protein 4 (ICP4) comprising fewer amino acids than a wild-type HSV ICP4.

In some embodiments, the modified HSV ICP4 comprises a truncation in a nuclear localization signal relative to the wild-type HSV ICP4.

In some embodiments, the modified HSV ICP4 does not comprise a nuclear localization signal.

In some embodiments, the modified HSV ICP4 comprises a truncation in a DNA-binding domain relative to the wild-type HSV ICP4.

In some embodiments, the modified HSV ICP4 does not comprise a DNA-binding domain.

In some embodiments, the modified HSV ICP4 comprises a linker between a first portion of the modified HSV ICP4 and a second portion of the modified HSV ICP4. In some embodiments, the linker comprises 2-10 glycine residues.

In some embodiments, the modified HSV ICP4 comprises an amino acid sequence having a length that is no more than 65% the length of the wild-type HSV IPC4.

In some embodiments, the modified HSV ICP4 amino acid sequence comprises at least 65% as many T cell epitopes as the wild-type HSV ICP4.

In some embodiments, the modified HSV ICP4 comprises about 687 amino acids.

Some aspects relate to a ribonucleic acid (RNA) comprising an open reading frame encoding the modified HSV ICP4 of any one of the preceding aspects or embodiments.

Other aspects relate to a herpes simplex virus (HSV) protein comprising an amino acid sequence having at least 90%, at least 95%, at least 96%, at least 97%, at least 98%, at least 99%, or 100% identity to the amino acid sequence of any one of SEQ ID NOs: 54, 63, 47, and 49.

Yet other aspects relate to a messenger ribonucleic acid (mRNA) comprising an open reading frame encoding the HSV protein of any one of the preceding aspects or embodiments.

Still other aspects relate to a messenger ribonucleic acid (mRNA) comprising an open reading frame comprising a nucleic acid sequence having at least 90%, at least 95%, at least 96%, at least 97%, at least 98%, at least 99%, or 100% identity to the nucleic acid sequence of any one of SEQ ID NOs: 19, 28, 4, 12, and 14.

In some embodiments, the mRNA comprises a chemical modification.

In some embodiments, 100% of the uracil nucleotides of the mRNA comprises a chemical modification.

In some embodiments, the chemical modification is 1-methylpseudouracil.

### BRIEF DESCRIPTION OF THE DRAWINGS

FIG. 1 shows the variant HSV proteins encoded by mRNAs of vaccine compositions evaluated in Example 1. SEQ ID NO: 78 (GGGSGGG) is shown.

FIGS. 2A-2F show antibody responses in mice vaccinated with compositions including mRNAs encoding HSV-2 gB, gC, gD, ICP0, and/or ICP4 that were evaluated in Example 1.

FIG. 2A shows titers of anti-gB IgG in sera. FIG. 2B shows titers of anti-gC IgG in sera. FIG. 2C shows titers of anti-gD IgG in sera. FIG. 2D shows neutralizing antibody titers towards strain MS of HSV-2. FIG. 2E shows the area under the curve of antibody-dependent cell-mediated cytotoxicity (ADCC) assays using HSV-1 KOS strain and sera collected on day 36.

FIG. 2F shows the extent to which sera blocked interactions between HSV-2 gC and complement protein C3b.

FIGS. 3A-3F show T cell responses in mice vaccinated with compositions including mRNAs encoding HSV-2 gB, gC, gD, ICP0, and/or ICP4 that were evaluated in Example 1.

FIG. 3A shows CD4+ T cell responses to peptides of gB, gC, and gD. FIG. 3B shows CD8+ T cell responses to peptides of gB, gC, and gD. FIG. 3C shows CD4+ T cell responses to peptides of gB, gC, and gD. FIG. 3D shows CD4+ T cell responses to peptides of ICP0 and ICP4. FIG. 3D shows CD8+ T cell responses to peptides of ICP0 and ICP4. FIG. 3E shows cumulative IFN-γ-producing CD4+ T cell responses across groups. FIG. 3F shows cumulative IFN-γ-producing CD8+ T cell responses across groups.

FIG. 4 shows the variant HSV proteins encoded by mRNAs of vaccine compositions evaluated an experiment described in Example 2. SEQ ID NO: 78 (GGGSGGG) is shown.

FIGS. 5A-5G show antibody responses in mice vaccinated with compositions including mRNAs encoding HSV-2 gB, gC, gD, ICP0, and ICP4 that were evaluated in Example 2. FIG. 5A shows titers of anti-gB IgG in sera. FIG. 5B shows titers of anti-gC IgG in sera. FIG. 5C shows titers of anti-gD IgG in sera. FIG. 5D shows neutralizing antibody titers towards strain MS of HSV-2. FIG. 5E shows neutralizing antibody titers towards strain KOS of HSV-1. FIG. 5F shows the area under the curve of antibody-dependent cell-mediated cytotoxicity (ADCC) assays using HSV-1 KOS strain and sera collected on day 36. FIG. 5G shows the extent to which sera blocked interactions between HSV-2 gC and complement protein C3b.

FIGS. 6A-6F show T cell responses in mice vaccinated with compositions including mRNAs encoding HSV-2 gB, gC, gD, ICP0, and ICP4 that were evaluated in Example 2. FIG. 6A shows CD4+ T cell responses to peptides of gB, gC, and gD. FIG. 6B shows CD8+ T cell responses to peptides of gB, gC, and gD. FIG. 6C shows CD4+ T cell responses to peptides of gB, gC, and gD. FIG. 6D shows CD4+ T cell responses to peptides of ICP0 and ICP4. FIG. 6D shows CD8+ T cell responses to peptides of ICP0 and ICP4. FIG. 6E shows cumulative IFN-γ-producing CD4+ T cell responses across groups. FIG. 6F shows cumulative IFN-γ-producing CD8+ T cell responses across groups.

FIGS. 7A-7E show antibody responses in mice vaccinated with compositions including mRNAs encoding HSV-2 gB, gC, gD, ICP0, and ICP4, and varying in their untranslated regions (UTRs), that were evaluated in Example 2. FIG. 7A shows titers of anti-gB IgG in sera. FIG. 7B shows titers of anti-gC IgG in sera. FIG. 7C shows titers of anti-gD IgG in sera. FIG. 7D shows neutralizing antibody titers towards strain MS of HSV-2. FIG. 7E shows the area under the curve of antibody-dependent cell-mediated cytotoxicity (ADCC) assays using HSV-1 KOS strain and sera collected on day 36.

FIGS. 8A-8B show T cell responses in mice vaccinated with compositions including mRNAs encoding HSV-2 gB, gC, gD, ICP0, and ICP4, and varying in their untranslated regions (UTRs), that were evaluated in Example 2. FIG. 8A shows CD4+ T cell responses to peptides of gB, gC, gD, ICP0, or ICP4. FIG. 8B shows CD8+ T cell responses to peptides of gB, gC, gD, ICP0, or ICP4.

FIG. 9 shows an overview of the inoculation, dosing, and sample collection schedule of an HSV challenge, treatment, and monitoring study in guinea pigs. Guinea pigs are inoculated with HSV-2 on day 0 and monitored for 14 days during the acute stage of infection. On days 21 and 35, guinea pigs are administered PBS as negative control, a composition containing mRNAs encoding HSV proteins, or another comparator vaccine as positive control. Vaginal lesions are monitored daily.

FIGS. 10A-10D show the design of mRNAs encoding truncated intracellular proteins (ICP) of HSV and the immunogenicity of truncated ICPs. FIG. 10A shows the location of epitopes in HSV ICP0, as well as the regions of HSV ICP0 that were encoded by mRNAs. FIG. 10B shows the location of epitopes in HSV ICP4, as well as the regions of ICP4 that were encoded by mRNAs. FIGS. 10C-10D show CD4+(FIG. 10D) and CD8+(FIG. 10D) T cell responses of mice immunized with two doses of mRNAs encoding ICP0 and/or ICP4.

FIGS. 11A-11D show in vitro expression of wild-type and modified HSV-2 glycoproteins. FIG. 11A shows total expression intensity of different forms of HSV-2 gB (measured by % of cells expressing gB*median fluorescence intensity of gB+ cells). FIG. 11B shows the frequency of cells expressing different forms of HSV-2 gC following transfection.

FIG. 11C shows the median fluorescence intensity of gC+ cells. FIG. 11D shows total expression intensity (measured by % of cells expressing gB*MFI of gB+ cells).

FIGS. 12A-12C show an overview of HSV antigens encoded by mRNAs of nucleic acid vaccines. FIG. 12A shows HSV proteins and variants that may be encoded by nucleic acid vaccines. FIG. 12B shows flow cytometry data related to expression of gB by cells transfected with mRNA encoding WT HSV gB (left) or pre-fusion HSV gB (right). FIG. 12C shows mean fluorescence intensity (MFI) of cells transfected with mRNA encoding WT HSV gB (left) or pre-fusion HSV gB (right), incubated with sera from mice immunized with PBS control, mRNA encoding WT gB, or pre-fusion gB, then stained with labeled anti-mouse IgG.

FIGS. 13A-13I show the variant HSV proteins encoded by mRNAs of nucleic acid vaccines provided herein. FIG. 13A shows variants of gB. FIG. 13B shows variants of gC. FIG. 13C shows variants of gD. FIG. 13D shows variants of gE. FIG. 13E shows variants of gH, including a gH covalently linked to gL. SEQ ID NO: 121 (GSGGSGSGGSSGGGSGSGGSGGSGSGGRRRRR) is shown. FIG. 13F shows variants of gL. FIG. 13G shows variants of gI. FIG. 13H shows variants of ICP0. FIG. 13I shows variants of HSV ICP4. SEQ ID NO: 78 (GGGSGGG) is shown.

FIGS. 14A-14C show the immunogenicity of a panel of vaccines containing mRNAs encoding prefusion H510P mutant of gB (gBpf), wild-type gB (gBwt), a C3b-binding F327A mutant of gC (gCmut), gD, soluble gE (sgE), or gH and gL (gHgL). FIGS. 14A-14B show neutralizing antibody titers towards strain F of HSV-1 (FIG. 14A) or strain MS of HSV-2 (FIG. 14B) in sera collected on day 36. FIG. 14C shows the area under the curve of antibody-dependent cell-mediated cytotoxicity (ADCC) assays using sera collected on day 36.

FIGS. 15A-15L show the antiviral activities of sera collected from mice vaccinated with one of a panel of vaccines including mRNA encoding HSV ICP4, HSV ICP0 and HSV ICP4, or the combination of gB, gC, and gD and optionally one or more other proteins. FIG. 15A shows neutralizing antibody titers towards HSV-1 (left two bars) and HSV-2 (right two bars) in sera collected from mice vaccinated with mRNA encoding gCmut, gD, and either gBwt or gBpf. FIGS. 15B-15C show neutralizing antibody titers towards strain F of HSV-1 (FIG. 15B) or strain MS of HSV-2 (FIG. 15C) in sera collected on day 36. FIG. 15D shows neutralizing antibody titers towards HSV-1 (left four bars) and HSV-2 (right four bars) in sera collected from mice vaccinated with mRNA encoding gBwt, gCmut, and gD (Base), as well as sgE and/or gHgL. FIG. 15E shows neutralizing antibody titers towards HSV-1 (left five bars) and HSV-2 (right five bars) in sera collected from mice vaccinated with mRNA encoding HSV gBwt, gCmut, and gD (Base), as well as sgE, gHgL, and/or ICP4. FIG. 15F shows neutralizing antibody titers towards HSV-1 (left five bars) and HSV-2 (right five bars) in sera collected from mice vaccinated with mRNA encoding HSV gBwt, gCmut, and gD (Base), as well as sgE, gHgL, ICP0, and/or ICP4. FIGS. 15G-15H show neutralizing antibody titers towards strain F of HSV-1 (FIG. 15G) or strain MS of HSV-2 (FIG. 15H) in sera collected on day 36, when sera were supplemented with guinea pig complement to a final concentration of 2.5% (v/v). FIGS. 15I-15J show neutralizing antibody titers towards strain F of HSV-1 (FIG. 15I) or strain MS of HSV-2 (FIG. 15J) in sera collected on day 36, when sera were supplemented with hyperimmune sera raised in mice by immunization with soluble gE prior to the neutralization assay. FIG. 15K shows neutralization titers against strain F of HSV-1 in a cell-cell spreading assay. FIG. 15L shows the area under the curve of antibody-dependent cell-mediated cytotoxicity (ADCC) assays using sera collected on day 36.

FIGS. 16A-16E show HSV protein-specific antibody titers in sera collected from mice vaccinated with one of a panel of vaccines including mRNA encoding (a) HSV ICP4, (b) HSV ICP0 and HSV ICP4, or (c) the combination of gB, gC, and gD and optionally one or more other proteins. FIG. 16A shows titers of anti-gB IgG in sera. FIG. 16B shows titers of anti-gC IgG in sera. FIG. 16C shows titers of anti-gD IgG in sera. FIG. 16D shows titers of anti-gHgL IgG in sera. FIG. 16E shows titers of anti-gE and anti-gI IgG in sera.

FIGS. 17A-17D show the T cell responses in mice vaccinated with one of a panel of vaccines including mRNA encoding HSV ICP4, HSV ICP0 and HSV ICP4, or the combination of gB, gC, and gD (also referred to as gD2) and optionally one or more other proteins. Mice were given two vaccine doses, one on days 0 and 22, then euthanized on day 36, two weeks after the second dose, to collect spleens for analysis of T cell responses. FIG. 17A shows the percentage of lymphocytes that were viable in each group. FIG. 17B shows the percentage of CD8+ T cells that were specific to HSV gB, as measured by pentamer staining. FIG. 17C shows cytokine responses by gD-specific CD4+ and CD8+ T cells from intracellular cytokine staining (ICS) analysis. FIG. 17D shows analysis of gL-specific CD4+ and CD8+ T cells from ICS analysis.

FIGS. 18A-18F show antiviral activities of sera collected from mice vaccinated with one of a panel of vaccines including different doses of mRNA encoding the combination of gB, gC, and gD and optionally one or more other proteins. Mice were administered two doses of the same mRNA vaccine on days 0 and 22, with sera collected on day 21, three weeks after administration of the first dose, and day 36, 14 days after administration of the second dose. FIGS. 18A-18B show neutralizing antibody titers towards HSV-1 F strain in day 36 sera from mice immunized with compositions containing 2 μg mRNA per antigen (FIG. 18A) or 0.4 μg mRNA per antigen (FIG. 18B). FIGS. 18C-18D show neutralizing antibody titers towards HSV-2 MS strain in day 36 sera from mice immunized with compositions containing 2 μg mRNA per antigen (FIG. 18C) or 0.4 μg mRNA per antigen (FIG. 18D). FIGS. 18E-18F show ADCC activity towards cells infected with HSV-1 KOS strain using day 36 sera from mice immunized with compositions containing 2 μg mRNA per antigen (FIG. 18E) or 0.4 μg mRNA per antigen (FIG. 18F).

FIGS. 19A-19E show HSV protein-specific antibody titers in sera collected from mice vaccinated with one of a panel of vaccines including different doses of mRNA encoding the combination of gB, gC, and gD and optionally one or more other proteins. Mice were administered two doses of the same mRNA vaccine on days 0 and 22, with sera collected on day 21, three weeks after administration of the first dose, and day 36, 14 days after administration of the second dose. FIG. 19A shows titers of anti-gB IgG in sera. FIG. 19B shows titers of anti-gC IgG in sera. FIG. 19C shows titers of anti-gD IgG in sera. FIG. 19D shows titers of anti-gE/gI IgG in sera. FIG. 19E shows titers of anti-gHgL IgG in sera.

FIG. 20 shows the T cell responses in mice vaccinated with one of a panel of vaccines including mRNA encoding the combination of gB, gC, and gD and optionally one or more other proteins. Mice were immunized with two doses of a given vaccine, one administered on day 0 and the other administered on day 22, then euthanized on day 36, two weeks after the second dose, to collect spleens for analysis of T cell responses. The percentage of CD8+ T cells that were specific to the SSIEFARL (SEQ ID NO: 103) epitope of HSV gB is shown.

FIGS. 21A-21G show HSV protein-specific antibody titers in, and antiviral activities of, sera collected from mice vaccinated with one of a panel of vaccines including different doses of mRNA encoding the combination of gB, gC, and gD and optionally one or more other proteins. Mice were administered two doses of the same mRNA vaccine on days 0 and 22, with sera collected on day 21, three weeks after administration of the first dose, and day 36, 14 days after administration of the second dose. FIG. 21A shows titers of anti-gB IgG in sera. FIG. 21B shows titers of anti-gC IgG in sera. FIG. 21C shows titers of anti-gD IgG in sera. FIG. 21D shows titers of anti-gE/gI IgG in sera. FIGS. 21E-21F show neutralizing antibody titers towards HSV-1 F strain (FIG. 21E) and HSV-2 MS strain (FIG. 21F) in day 36 sera. FIG. 21G shows ADCC activity towards cells infected with HSV-1 KOS strain using day 36 sera.

FIGS. 22A-22D show the antiviral activities of sera collected from mice vaccinated with one of a panel of vaccines including mRNA encoding HSV-2 gC, gD, and a variant gB. FIGS. 22A-22B show neutralizing antibody titers towards strain F of HSV-1 (FIG. 22A) or strain MS of HSV-2 (FIG. 22B) in sera collected on day 36. FIG. 22C shows the area under the curve of antibody-dependent cell-mediated cytotoxicity (ADCC) assays using sera collected on day 36. FIG. 22D shows neutralizing antibody titers towards the MS strain of HSV-2 in sera collected on day 36 from mice immunized in a follow-up experiment using mRNA encoding a modified HSV-2 gB.

FIGS. 23A-23B show the antiviral activities of sera collected from mice vaccinated with one of a panel of vaccines including mRNA encoding HSV-2 gB, gC, gD, sgE, variants of gL, and optionally gH, FIG. 23A shows neutralizing antibody titers towards strain MS of HSV-2. FIG. 23B shows the area under the curve of antibody-dependent cell-mediated cytotoxicity (ADCC) assays using sera collected on day 36.

FIGS. 24A-24C show the antiviral activities of sera collected from mice immunized with one of a panel of vaccines containing individual mRNAs encoding HSV-2 gB, gC, gD, or sgE, or compositions containing various ratios of mRNAs encoding gB, gC, and gD, and optionally sgE. FIG. 24A shows neutralizing antibody titers towards strain MS of HSV-2. FIG. 24B shows the area under the curve of antibody-dependent cell-mediated cytotoxicity (ADCC) assays using HSV-1 KOS strain and sera collected on day 36. FIG. 24C shows neutralizing antibody titers in sera collected on day 36 from mice immunized in a follow-up experiment using varying ratios of mRNA encoding HSV-2 gB.

FIGS. 25A-25C show antibody and T cell responses in mice vaccinated with one of a panel of vaccines including mRNAs encoding HSV-2 gB, gC, gD, ICP0, and ICP4. FIG. 25A shows neutralizing antibody titers towards the MS strain of HSV-2 in sera collected on day 36. FIG. 25B shows CD4+ T cell responses in mice. FIG. 25C shows the extent to which sera blocked interactions between HSV-2 gC and complement protein C3b.

FIGS. 26A-26C show antibody and T cell responses in mice vaccinated with compositions including mRNAs encoding HSV-2 gB, gC, gD, ICP0, and ICP4. FIG. 26A shows neutralizing antibody titers towards strain MS of HSV-2. FIG. 26B shows the area under the curve of antibody-dependent cell-mediated cytotoxicity (ADCC) assays using HSV-1 KOS strain and sera collected on day 36. FIG. 26C shows CD4+ and CD8+ T cell responses in mice of group 2.

### DETAILED DESCRIPTION

Provided herein are messenger ribonucleic acid (mRNA) vaccines that build on the knowledge that modified mRNA can safely direct the body's cellular machinery to produce nearly any protein of interest, from native proteins to antibodies and other entirely novel protein constructs that can have therapeutic activity inside and outside of cells. The RNA (e.g., mRNA) vaccines of the present disclosure may be used to induce a balanced immune response against herpes simplex virus (HSV), comprising both cellular and humoral immunity, without risking the possibility of insertional mutagenesis, for example. While not wishing to be bound by theory, it is believed that the RNA vaccines, as mRNA polynucleotides, are better designed to produce the appropriate protein conformation upon translation as the RNA vaccines co-opt natural cellular machinery. Unlike traditional vaccines which are manufactured ex vivo and may trigger unwanted cellular responses, the RNA vaccines are presented to the cellular system in a more native fashion.

### Herpes Simplex Virus Proteins

Some aspects of the present disclosure provide vaccines that include RNA (e.g., mRNA) comprising an open reading frame encoding a herpes simplex virus (HSV) protein. HSV is a double-stranded, linear DNA virus in the Herpesviridae. Two members of the herpes simplex virus family infect humans—known as HSV-1 and HSV-2. Symptoms of HSV infection include the formation of blisters in the skin or mucous membranes of the mouth, lips and/or genitals. HSV is a neuroinvasive virus that can cause sporadic recurring episodes of viral reactivation in infected individuals. HSV is transmitted by contact with an infected area of the skin during a period of viral activation. HSV most commonly infects via the oral or genital mucosa and replicates in the stratified squamous epithelium, followed by uptake into ramifying unmyelinated sensory nerve fibers within the stratified squamous epithelium. The virus is then transported to the cell body of the neuron in the dorsal root ganglion, where it persists in a latent cellular infection (Cunningham AL et al. J Infect Dis. (2006) 194 (Supplement 1): S11-S18).

The terms “naturally occurring” and “wild type” are used interchangeably herein. A naturally occurring HSV protein is an unmodified HSV protein of a herpes simplex virus (e.g., HSV-1 or HSV-2) that occurs in nature, i.e., which is a naturally occurring isolate. As is known in the art, a naturally occurring protein is not genetically engineered. A naturally occurring protein is not genetically (or otherwise) modified to substitute, remove, or add any amino acids. In some embodiments, the naturally occurring isolate of HSV is HSV-2 strain HG52 (GenBank Accession No. Z86099.2). Amino acid sequences of gB, gC, gC, ICP0, and ICP4 of HSV-2 strain HG52 are provided in UniProt Accession Nos. P08666, Q89730, Q69467, P28284, and P90493, respectively.

In some embodiments, a wild-type HSV gB comprises the amino acid sequence of SEQ ID NO: 36. In some embodiments, a wild-type HSV gC comprises the amino acid sequence of SEQ ID NO: 65. In some embodiments, a wild-type HSV gD comprises the amino acid sequence of SEQ ID NO: 39. In some embodiments, a wild-type HSV gB comprises the amino acid sequence of SEQ ID 112. In some embodiments, a wild-type HSV gC comprises the amino acid sequence of SEQ ID NO: 114. In some embodiments, a wild-type HSV gD comprises the amino acid sequence of SEQ ID NO: 116. In some embodiments, a wild-type HSV ICP0 comprises the amino acid sequence of SEQ ID NO: 118. In some embodiments, a wild-type HSV ICP4 comprises the amino acid sequence of SEQ ID NO: 120. Wild-type nucleic acid and/or protein sequences may be obtained, for example, by sequencing the genome or certain genes of one or more viral isolates, and/or proteins expressed by the genome or certain genes of one or more of the viral isolates. Some aspects of the present disclosure provide vaccines comprising RNA (e.g., mRNA) having an open reading frames that encode multiple HSV antigens, including HSV glycoprotein B (gB), glycoprotein C (gC), and glycoprotein D (gD). In some embodiments, a vaccine comprises a first RNA (e.g., mRNA) comprising an open reading frame encoding an HSV glycoprotein B (gB), a second RNA (e.g., mRNA) comprising an open reading frame encoding an HSV glycoprotein C (gC), and a third RNA (e.g., mRNA) comprising an open reading frame encoding an HSV glycoprotein D (gD). Expression of gB, gC, and gD by cells containing the RNA elicits gB-, gC-, and gD-specific antibodies and T cells. Because HSV glycoproteins C, B, and D are required for viral entry, such trivalent vaccines are useful in generating potent neutralizing antibody responses that prevent or limit HSV infection. Additionally, antigens encoded by RNAs of the vaccines may be modified relative to wild-type HSV antigens to improve the antibody responses elicited by immunization (e.g., by stabilizing HSV gB in a prefusion state) or prevent the deleterious effects (e.g., complement sequestration or masking of epitopes by bound complement) of HSV protein expression.

Glycoprotein B (gB) is a viral glycoprotein involved in the viral cell activity of herpes simplex virus (HSV) and is required for the fusion of the HSV's envelope with the cellular membrane. It is the most highly conserved of all surface glycoproteins and primarily acts as a fusion protein, constituting the core fusion machinery. gB, a class III membrane fusion glycoprotein, is a type-1 transmembrane protein trimer of five structural domains. Domain I includes two internal fusion loops and is thought to insert into the cellular membrane during virus-cell fusion. Domain II appears to interact with gH/gL during the fusion process, domain III contains an elongated alpha helix, and domain IV interacts with cellular receptors. In some embodiments, an mRNA of an HSV vaccine of the present disclosure encodes an HSV-1 gB. In other embodiments, an mRNA of an HSV vaccine of the present disclosure encodes an HSV-2 gB. In some embodiments, a wild-type HSV-1 gB comprises the amino acid sequence of SEQ ID NO: 111. In some embodiments, a wild-type HSV-2 gB comprises the amino acid sequence of SEQ ID NO: 112.

In some embodiments, an mRNA of an HSV vaccine of the present disclosure encodes an HSV gB with a truncated C-terminus. An HSV gB with a truncated C-terminus refers to an HSV gB that lacks one or more amino acids that are present at the C-terminus of a wild-type HSV gB. In some embodiments, an encoded HSV gB comprises a truncated cytoplasmic tail. In other embodiments, the HSV gB does not comprise a cytoplasmic tail. For example, a wild-type HSV-2 gB having the amino acid sequence of SEQ ID NO: 112 (Accession No. P06763) comprises a cytoplasmic tail that is 112 amino acids long (amino acids 793-904 of SEQ ID NO: 112), and a so a modified HSV-2 gB with a truncated C-terminus relative to SEQ ID NO: 112 comprises (i) a cytoplasmic tail with having fewer than 112 amino acids; or (ii) no cytoplasmic tail. Similarly, a wild-type HSV-1 gB having the amino acid sequence of SEQ ID NO: 111 (Accession No. P10211) comprises a cytoplasmic tail that is 109 amino acids long (amino acids 796-904 of SEQ ID NO: 111), and a so a modified HSV-2 gB with a truncated C-terminus relative to SEQ ID NO: 111 comprises (i) a cytoplasmic tail with having fewer than 109 amino acids; or (ii) no cytoplasmic tail.

In some embodiments, an HSV gB comprises a cytoplasmic tail that comprises no more than 105, no more than 100, no more than 90, no more than 80, no more than 70, no more than 60, no more than 50, no more than 40, no more than 30, no more than 25, no more than 20, no more than 15, no more than 10, no more than 9, no more than 8, no more than 7, no more than 6, no more than 5, no more than 4, no more than 3, no more than 2, or no more than 1 amino acid. In some embodiments, an HSV gB comprises a cytoplasmic tail that is 1, 2, 3, 4, 5, 6, 7, 8, 9, or 10 amino acids in length. In some embodiments, an HSV gB comprises a cytoplasmic tail that is 10-20, 20-30, 30-40, or 40-50 amino acids in length. In some embodiments, the HSV gB comprises a cytoplasmic tail comprising no more than 20 amino acids. In some embodiments, the HSV gB comprises a cytoplasmic tail comprising no more than 10 amino acids. In some embodiments, the HSV gB comprises a cytoplasmic tail comprising no more than 8 amino acids. In some embodiments, the HSV gB comprises a cytoplasmic tail comprising no more than 6 amino acids. In some embodiments, the HSV gB comprises a cytoplasmic tail comprising no more than 5 amino acids. In some embodiments, the HSV gB comprises a cytoplasmic tail comprising no more than 4 amino acids. In some embodiments, the HSV gB comprises a cytoplasmic tail comprising no more than 3 amino acids. In some embodiments, the HSV gB comprises a cytoplasmic tail comprising no more than 2 amino acids. In some embodiments, the HSV gB comprises a cytoplasmic tail comprising no more than 1 amino acid.

Truncations may be introduced by deleting one or more amino acids from the C-terminus of the cytoplasmic tail (e.g., amino acids 796-904 of SEQ ID NO: 112 or amino acids 793-904 of SEQ ID NO: 111). In some embodiments, the HSV gB encoded by an mRNA of a vaccine of the present disclosure lacks one or more amino acids present at the C-terminus of a wild-type sequence. In some embodiments, the encoded HSV gB lacks 1-112, 10-112, 20-112, 30-112, 40-112, 50-112, 60-112, 70-112, 80-112, 90-112, 100-112, 1-109, 10-109, 20-109, 30-109, 40-109, 50-109, 60-109, 70-109, 80-109, 90-109, 100-109, 1-103, 10-103, 20-103, 30-103, 40-103, 50-103, 60-103, 70-103, 80-103, 90-103, or 100-103 amino acids that are present at the C-terminus of a wild-type gB amino acid sequence.

In some embodiments, an mRNA of the present disclosure encodes an HSV gB that does not comprise a cytoplasmic tail. In some embodiments, the HSV gB comprises an extracellular domain and a transmembrane domain, and does not comprise a cytoplasmic tail.

Glycoprotein C (gC) is a glycoprotein involved in viral attachment to host cells; e.g., it acts as an attachment protein that mediates binding of the HSV-2 virus to host adhesion receptors, namely cell surface heparan sulfate and/or chondroitin sulfate. gC plays a role in host immune evasion (aka viral immunoevasion) by inhibiting the host complement cascade activation. In particular, gC binds to and/or interacts with host complement component C3b; this interaction then inhibits the host immune response by dysregulating the complement cascade (e.g., binds host complement C3b to block neutralization of virus).

In some embodiments, an mRNA of an HSV vaccine of the present disclosure encodes an HSV-1 gC. In other embodiments, an mRNA of an HSV vaccine of the present disclosure encodes an HSV-2 gC. In some embodiments, a wild-type HSV-1 gC comprises the amino acid sequence of SEQ ID NO: 113. In some embodiments, a wild-type HSV-2 gC comprises the amino acid sequence of SEQ ID NO: 114.

In some embodiments, an mRNA of an HSV vaccine of the present disclosure encodes an HSV gC with a truncated C-terminus. An HSV gC with a truncated C-terminus refers to an HSV gC that lacks one or more amino acids that are present at the C-terminus of a wild-type HSV gC. In some embodiments, an encoded HSV gC comprises a truncated cytoplasmic tail. In other embodiments, the HSV gC does not comprise a cytoplasmic tail. For example, a wild-type HSV-2 gC having the amino acid sequence of SEQ ID NO: 114 (Accession No. Q89730) comprises a cytoplasmic tail that is 12 amino acids long (amino acids 469-480 of SEQ ID NO: 114), and a so a modified HSV-2 gC with a truncated C-terminus relative to SEQ ID NO: 114 comprises (i) a cytoplasmic tail with having fewer than 12 amino acids; or (ii) no cytoplasmic tail. Similarly, a wild-type HSV-1 gC having the amino acid sequence of SEQ ID NO: 113 (Accession No. Q8UZ70) comprises a cytoplasmic tail that is 11 amino acids long (amino acids 501-511 of SEQ ID NO: 113), and a so a modified HSV-2 gC with a truncated C-terminus relative to SEQ ID NO: 113 comprises (i) a cytoplasmic tail with having fewer than 11 amino acids; or (ii) no cytoplasmic tail.

In some embodiments, an HSV gC comprises a cytoplasmic tail that comprises no more than 10, no more than 9, no more than 8, no more than 7, no more than 6, no more than 5, no more than 4, no more than 3, no more than 2, or no more than 1 amino acid. In some embodiments, an HSV gC comprises a cytoplasmic tail that is 1, 2, 3, 4, 5, 6, 7, 8, 9, or 10 amino acids in length. In some embodiments, an HSV gC comprises a cytoplasmic tail that is 1-2, 3-4, 4-5, 6-7, 8-9, or 9-10 amino acids in length. In some embodiments, the HSV gC comprises a cytoplasmic tail comprising no more than 10 amino acids. In some embodiments, the HSV gC comprises a cytoplasmic tail comprising no more than 9 amino acids. In some embodiments, the HSV gC comprises a cytoplasmic tail comprising no more than 8 amino acids. In some embodiments, the HSV gC comprises a cytoplasmic tail comprising no more than 7 amino acids. In some embodiments, the HSV gC comprises a cytoplasmic tail comprising no more than 6 amino acids. In some embodiments, the HSV gC comprises a cytoplasmic tail comprising no more than 5 amino acids. In some embodiments, the HSV gC comprises a cytoplasmic tail comprising no more than 4 amino acids. In some embodiments, the HSV gC comprises a cytoplasmic tail comprising no more than 3 amino acids. In some embodiments, the HSV gC comprises a cytoplasmic tail comprising no more than 2 amino acids. In some embodiments, the HSV gC comprises a cytoplasmic tail comprising no more than 1 amino acid.

Truncations may be introduced by deleting one or more amino acids at any position in the cytoplasmic tail (e.g., amino acids 469-480 of SEQ ID NO: 114 or amino acids 501-511 of SEQ ID NO: 113). In some embodiments, the HSV gC encoded by an mRNA of a vaccine of the present disclosure lacks one or more amino acids present at the C-terminus of a wild-type sequence. In some embodiments, the encoded HSV gC lacks 1-10, 1-9, 1-8, 1-7, 1-6, 1-5, 1-4, 1-3, or 1-2 amino acids that are present at the C-terminus of a wild-type gC amino acid sequence.

In some embodiments, an mRNA of the present disclosure encodes an HSV gC that does not comprise a cytoplasmic tail. In some embodiments, the HSV gC comprises an extracellular domain and a transmembrane domain, and does not comprise a cytoplasmic tail.

In some embodiments, an HSV gC encoded by an mRNA of a vaccine of the present disclosure comprises a substitution at a residue corresponding to F327 of a wild-type HSV gC. In some embodiments, the HSV gC comprises an alanine (A) at a position corresponding to a wild-type HSV gC. In some embodiments, the HSV gC comprises an aliphatic amino acid at a residue corresponding to amino acid 327 of a wild-type HSV gC. Aliphatic amino acids are known in the art, and include glycine (G), alanine (A), valine (V), leucine (L), and isoleucine. In some embodiments, the HSV gC comprises an F327A substitution at a residue corresponding to amino acid 327 of a wild-type HSV gC. In some embodiments, the HSV gC comprises an amino acid at a residue corresponding to amino acid 327 of the wild-type HSV gC that is not an aromatic amino acid. Aromatic amino acids are known in the art, and include phenylalanine (F), tyrosine (Y), and tryptophan (W). In some embodiments, an HSV gC comprising an amino acid that is not phenylalanine (F) at a residue corresponding to amino acid 327 of a wild-type HSV gC binds C3b with a lower affinity than the wild-type HSV gC. In some embodiments, an HSV gC comprising an amino acid that is not F, Y, or W at a residue corresponding to amino acid 327 of a wild-type HSV gC binds C3b with a lower affinity than the wild-type HSV gC.

Glycoprotein D (gD) is an envelope glycoprotein that binds to cell surface receptors and/or is involved in cell attachment via poliovirus receptor-related protein and/or herpesvirus entry mediator, facilitating virus entry. gD binds to the potential host cell entry receptors (tumor necrosis factor receptor superfamily, member 14(TNFRSF14)/herpesvirus entry mediator (HVEM), poliovirus receptor-related protein 1 (PVRL1) and or poliovirus receptor-related protein 2 (PVRL2) and is proposed to trigger fusion with host membrane by recruiting the fusion machinery composed of, for example, gB and gH/gL. gD interacts with host cell receptors TNFRSF14 and/or PVRL1 and/or PVRL2 and (1) interacts (via profusion domain) with gB; an interaction which can occur in the absence of related HSV glycoproteins, e.g., gH and/or gL; and (2) gD interacts (via profusion domain) with gH/gL heterodimer, an interaction which can occur in the absence of gB. As such, gD associates with the gB-gH/gL-gD complex. gD also interacts (via C-terminus) with UL11 tegument protein.

In some embodiments, an mRNA of an HSV vaccine of the present disclosure encodes an HSV-1 gD. In other embodiments, an mRNA of an HSV vaccine of the present disclosure encodes an HSV-2 gD. In some embodiments, a wild-type HSV-1 gD comprises the amino acid sequence of SEQ ID NO: 115. In some embodiments, a wild-type HSV-2 gD comprises the amino acid sequence of SEQ ID NO: 116.

In some embodiments, an mRNA of an HSV vaccine of the present disclosure encodes an HSV gD with a truncated C-terminus. An HSV gD with a truncated C-terminus refers to an HSV gD that lacks one or more amino acids that are present at the C-terminus of a wild-type HSV gD. In some embodiments, an encoded HSV gD comprises a truncated cytoplasmic tail. In other embodiments, the HSV gD does not comprise a cytoplasmic tail. For example, a wild-type HSV-2 gD having the amino acid sequence of SEQ ID NO: 116 (Accession No. P03172) comprises a cytoplasmic tail that is 30 amino acids long (amino acids 364-393 of SEQ ID NO: 116), and a so a modified HSV-2 gD with a truncated C-terminus relative to SEQ ID NO: 116 comprises (i) a cytoplasmic tail with having fewer than 30 amino acids; or (ii) no cytoplasmic tail, while an HSV-2 gD with a wild-type cytoplasmic tail comprises all 30 amino acids corresponding to amino acids 364-393 of SEQ ID NO: 116. Similarly, a wild-type HSV-1 gD having the amino acid sequence of SEQ ID NO: 115 (Accession No. Q69091) comprises a cytoplasmic tail that is 33 amino acids long (amino acids 362-394 of SEQ ID NO: 115), and a so a modified HSV-2 gD with a truncated C-terminus relative to SEQ ID NO: 115 comprises (i) a cytoplasmic tail with having fewer than 33 amino acids; or (ii) no cytoplasmic tail, while an HSV-2 gD with a wild-type cytoplasmic tail comprises all 33 amino acids corresponding to amino acids 362-394 of SEQ ID NO: 115.

In some embodiments, an HSV gD comprises a cytoplasmic tail that comprises no more than 35, no more than 30, no more than 25, no more than 20, no more than 15, no more than 10, no more than 9, no more than 8, no more than 7, no more than 6, no more than 5, no more than 4, no more than 3, no more than 2, or no more than 1 amino acid. In some embodiments, an HSV gD comprises a cytoplasmic tail that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 amino acids in length. In some embodiments, an HSV gD comprises a cytoplasmic tail that is 1-2, 3-4, 4-5, 6-7, 8-9, 9-10, 10-15, 15-20, 20-25, 25-30, or 30-35 amino acids in length. In some embodiments, the HSV gD comprises a cytoplasmic tail comprising no more than 10 amino acids. In some embodiments, the HSV gD comprises a cytoplasmic tail comprising no more than 9 amino acids. In some embodiments, the HSV gD comprises a cytoplasmic tail comprising no more than 8 amino acids. In some embodiments, the HSV gD comprises a cytoplasmic tail comprising no more than 7 amino acids. In some embodiments, the HSV gD comprises a cytoplasmic tail comprising no more than 6 amino acids. In some embodiments, the HSV gD comprises a cytoplasmic tail comprising no more than 5 amino acids. In some embodiments, the HSV gD comprises a cytoplasmic tail comprising no more than 4 amino acids. In some embodiments, the HSV gD comprises a cytoplasmic tail comprising no more than 3 amino acids. In some embodiments, the HSV gD comprises a cytoplasmic tail comprising no more than 2 amino acids. In some embodiments, the HSV gD comprises a cytoplasmic tail comprising no more than 1 amino acid.

Truncations may be introduced by deleting one or more amino acids at any position in the cytoplasmic tail (e.g., amino acids 469-480 of SEQ ID 116 or amino acids 501-511 of SEQ ID NO: 115). In some embodiments, the HSV gD encoded by an mRNA of a vaccine of the present disclosure lacks one or more amino acids present at the C-terminus of a wild-type sequence. In some embodiments, the encoded HSV gD lacks 1-10, 1-9, 1-8, 1-7, 1-6, 1-5, 1-4, 1-3, or 1-2 amino acids that are present at the C-terminus of a wild-type gD amino acid sequence.

In some embodiments, an mRNA of the present disclosure encodes an HSV gD that does not comprise a cytoplasmic tail. In some embodiments, the HSV gD comprises an extracellular domain and a transmembrane domain, and does not comprise a cytoplasmic tail.

In some embodiments, the vaccines provide herein further comprise RNA (e.g., mRNA) having an open reading frame that encodes HSV intracellular protein 0 (ICP0). In some embodiments, the vaccines provide herein further comprise RNA (e.g., mRNA) having an open reading frame that encodes HSV intracellular protein 4 (ICP4). Intracellular expression of HSV ICP0 and/or ICP4 promotes generation of ICP0- and ICP4-specific CD8+ T cells, respectively, which clear cells in which HSV is actively replicating and expressing ICP0 and ICP4. In some embodiments, the HSV ICP0 is a modified ICP0 comprising one or more internal deletions relative to a wild-type HSV ICP0 amino acid sequence. Such deletions may reduce the size of an ICP0, thereby increasing the number of ICP0 proteins that may be produced from a given amount of amino acids. In some embodiments, the modified HSV ICP0 comprises an amino acid sequence that is no more than 75%, 70% or 65% as long as a wild-type HSV ICP0 amino acid sequence. In some embodiments, the modified HSV ICP0 comprises no more than 600, 550, 500, or 487 amino acids. In some embodiments, modified HSV ICP0 comprises about 487 amino acids.

In some embodiments, the modified HSV ICP4 comprises an amino acid sequence that is no more than 75%, 70% or 65% as long as a wild-type HSV ICP4 amino acid sequence. In some embodiments, the modified HSV ICP0 comprises no more than 800, 750, 700, or 687 amino acids. In some embodiments, modified HSV ICP0 comprises about 687 amino acids.

In modified HSV ICP0 and/or ICP4, deletions of one or more regions of HSV ICP0 and/or ICP4 that contain few or no T cell epitopes may also increase the epitope density in a modified HSV ICP0 or ICP4, relative to a wild-type HSV ICP0 or ICP4 amino acid sequence, thereby enhancing the CD8+ T cell response elicited by a modified HSV ICP0 or ICP4. Deletions may also remove all or part of a domain of ICP0 or ICP4 to enhance immunogenicity and/or safety of the modified HSV ICP0 or ICP4. For example, a modified HSV ICP0 or ICP4 may comprise a deletion of a nuclear localization signal, promoting retention of the modified HSV ICP0 or ICP4 in the cytoplasm, where it can more readily be processed by the proteasome into epitopes for presentation to CD8+ T cells. As another example, the modified HSV ICP0 may comprise a deletion in a USP7-binding domain, which inhibits Toll-like receptor-mediated signaling and consequently hinders the innate immune response. See Daubeuf et al., Blood. 2009 113(114):3264-3275. In addition, the modified HSV ICP0 may comprise a deletion in a RING finger domain, which inhibits IRF3- and IRF7-mediated activation of interferon-stimulated genes. See Lin et al., J Virol. 2004. 78(4):1674-1684. In some embodiments, the modified HSV ICP4 comprises a deletion in a DNA-binding domain.

In some embodiments, an mRNA of an HSV vaccine of the present disclosure encodes an HSV-1 ICP0. In other embodiments, an mRNA of an HSV vaccine of the present disclosure encodes an HSV-2 ICP0. In some embodiments, a wild-type HSV-1 ICP0 comprises the amino acid sequence of SEQ ID NO: 117. In some embodiments, a wild-type HSV-2 ICP0 comprises the amino acid sequence of SEQ ID NO: 118.

In some embodiments, an mRNA of an HSV vaccine of the present disclosure encodes an HSV ICP0 with a truncation in one or more of a nuclear localization signal, RING finger domain, or USP7-binding domain. For example, an HSV-2 ICP0 having the amino acid sequence of SEQ ID NO: 118 (Accession No. P28284) comprises a nuclear localization signal at amino acids 468-549, a RING-finger domain at amino acids 124-176, and a USP7-binding domain at amino acids 660-665. See Halford et al., PLoS One. 2010. 5(8):e12251; and Pfoh et al., PLoS Pathog. 2015. 11(6):e1004950. Thus, in SEQ ID NO: 118, a nuclear localization signal corresponds to amino acids 468-549, and so a modified ICP0 having a truncation in a nuclear localization signal relative to SEQ ID NO: 118 lacks one or more amino acids corresponding to amino acids 468-549 of SEQ ID NO: 118. In SEQ ID NO: 118, a RING finger domain corresponds to amino acids 124-176, and so a modified ICP0 having a truncation in a RING finger domain relative to SEQ ID NO: 118 lacks one or more amino acids corresponding to amino acids 124-176 of SEQ ID NO: 118. In SEQ ID NO: 118, a USP7-binding domain corresponds to amino acids 660-665, and so a modified ICP0 having a truncation in a USP7-binding domain relative to SEQ ID NO: 118 lacks one or more amino acids corresponding to amino acids 660-665 of SEQ ID NO: 118.

Some portions of ICP0 that are shortened or removed by truncation (e.g., USP7-binding and/or RING finger domains, and/or nuclear localization signals) are located internally on a wild-type HSV ICP0, and so truncations in these domains are internal truncations. An HSV ICP0 comprising a truncation in one or more of these domains or signals is truncated internally, such that it lacks one or more amino acids that is present in the domain of a wild-type ICP0 sequence, but comprises one or more amino acids that flank the deleted amino acid(s) in the wild-type ICP0 sequence. Additionally, an HSV ICP0 encoded by an mRNA of a vaccine of the present disclosure may comprise a truncated C-terminus and/or a truncated N-terminus. In some embodiments, a modified HSV ICP0 lacks an amino acid sequence comprising 1-200, 1-190, 1-180, 1-170, 1-160, 1-150, 1-140, 1-130, 1-120, 1-110, 1-100, 1-90, 1-80, 1-70, 1-60, 1-50, 1-40, 1-30, 1-25, 1-20, 1-10, or 1-5, 10-200, 20-200, 30-200, 40-200, 50-200, 60-200, 70-200, 80-200, 90-200, 100-200, 110-200, 120-200, 130-200, 140-200, 150-200, 160-200, 170-200, 180-200, 190-200, 10-30, 30-50, 50-75, 75-100, 100-125, 125-150, 150-175, or 175-200 amino acids that is present that is present at the C-terminus of a wild-type ICP0 amino acid sequence. In some embodiments, a modified HSV ICP0 lacks an amino acid sequence comprising 1-200, 1-190, 1-180, 1-170, 1-160, 1-150, 1-140, 1-130, 1-120, 1-110, 1-100, 1-90, 1-80, 1-70, 1-60, 1-50, 1-40, 1-30, 1-25, 1-20, 1-10, or 1-5, 10-200, 20-200, 30-200, 40-200, 50-200, 60-200, 70-200, 80-200, 90-200, 100-200, 110-200, 120-200, 130-200, 140-200, 150-200, 160-200, 170-200, 180-200, 190-200, 10-30, 30-50, 50-75, 75-100, 100-125, 125-150, 150-175, or 175-200 amino acids that is present that is present at the N-terminus of a wild-type ICP0 amino acid sequence. In some embodiments, a modified HSV ICP0 lacks an amino acid sequence comprising 1-200, 1-190, 1-180, 1-170, 1-160, 1-150, 1-140, 1-130, 1-120, 1-110, 1-100, 1-90, 1-80, 1-70, 1-60, 1-50, 1-40, 1-30, 1-25, 1-20, 1-10, or 1-5, 10-200, 20-200, 30-200, 40-200, 50-200, 60-200, 70-200, 80-200, 90-200, 100-200, 110-200, 120-200, 130-200, 140-200, 150-200, 160-200, 170-200, 180-200, 190-200, 10-30, 30-50, 50-75, 75-100, 100-125, 125-150, 150-175, or 175-200 amino acids that is present between the N-terminal amino acid and C-terminal amino acid of a wild-type ICP0 amino acid sequence. In some embodiments, a modified HSV ICP0 lacks an amino acid sequence comprising 167 amino acids that is present at the N-terminus of a wild-type HSV ICP0. In some embodiments, a modified HSV ICP0 lacks an amino acid sequence comprising 12 amino acids that is present at the C-terminus of a wild-type HSV ICP0. In some embodiments, a modified HSV ICP0 lacks an amino acid sequence comprising 170 amino acids that is present between the N- and C-termini of a wild-type HSV ICP0.

In some embodiments, the modified HSV ICP0 comprises a truncated nuclear localization signal relative to a wild-type HSV ICP0. In some embodiments, the modified HSV ICP0 comprises a truncated nuclear localization signal comprising no more than 50%, no more than 40%, no more than 30%, no more than 25%, no more than 20%, no more than 15%, no more than 10%, or no more than 5% the length of the nuclear localization signal of the wild-type HSV ICP0. In some embodiments, the modified HSV ICP0 comprises a truncated nuclear localization signal comprising no more than 75, no more than 70, no more than 60, no more than 50, no more than 40, no more than 30, no more than 25, no more than 20, no more than 10, no more than 9, no more than 8, no more than 7, no more than 6, no more than 5, no more than 4, no more than 3, no more than 2, or no more than 1 amino acid. In some embodiments, the modified HSV ICP0 comprises a truncated nuclear localization signal comprising 1, 2, 3, 4, 5, 6, 7, 8, 9, 10, 11, 12, 13, 14, 15, 16, 17, 18, 19, 20, 21, 22, 23, 24, or 25 amino acids. In some embodiments, the truncated nuclear localization signal comprises 1-20, 1-15, 1-10, or 1-5 amino acids. In some embodiments, the truncated nuclear localization signal comprises 1-5, 1-4, 1-3, 1-2, or 1 amino acid. In some embodiments, the modified HSV ICP0 does not comprise a nuclear localization signal. In some embodiments, the modified HSV ICP0 is present in the cytoplasm following translation of the mRNA encoding the HSV ICP0. In some embodiments, the modified HSV ICP0 does not localize to the nucleus following translation of the mRNA encoding the HSV ICP0.

In some embodiments, the modified HSV ICP0 comprises a truncated RING finger domain relative to a wild-type HSV ICP0. In some embodiments, the modified HSV ICP0 comprises a RING finger domain comprising no more than 50%, no more than 40%, no more than 30%, no more than 25%, no more than 20%, no more than 15%, no more than 10%, or no more than 5% the length of the RING finger domain of the wild-type HSV ICP0. In some embodiments, the modified HSV ICP0 comprises a truncated RING finger domain comprising no more than 50, no more than 40, no more than 30, no more than 25, no more than 20, no more than 15, no more than 10, no more than 9, no more than 8, no more than 7, no more than 6, no more than 5, no more than 4, no more than 3, no more than 2, or no more than 1 amino acid. In some embodiments, the modified HSV ICP0 comprises a truncated RING finger domain comprising 1, 2, 3, 4, 5, 6, 7, 8, 9, 10, 11, 12, 13, 14, 15, 16, 17, 18, 19, 20, 21, 22, 23, 24, or 25 amino acids. In some embodiments, the truncated RING finger domain comprises 1-20, 1-15, 1-10, or 1-5 amino acids. In some embodiments, the truncated RING finger domain comprises 1-5, 1-4, 1-3, 1-2, or 1 amino acid.

In some embodiments, the modified HSV ICP0 comprises a truncated USP7-binding domain relative to a wild-type HSV ICP0. In some embodiments, the modified HSV ICP0 comprises a USP7-binding domain comprising no more than 50%, no more than 40%, no more than 30%, no more than 25%, no more than 20%, no more than 15%, no more than 10%, or no more than 5% the length of the USP7-binding domain of the wild-type HSV ICP0. In some embodiments, the modified HSV ICP0 comprises a truncated USP7-binding domain comprising no more than 5, no more than 4, no more than 3, no more than 2, or no more than 1 amino acid. In some embodiments, the modified HSV ICP0 comprises a truncated USP7-binding domain comprising 1, 2, 3, 4, or 5 amino acids. In some embodiments, the truncated USP7-binding domain comprises 1-5 amino acids. In some embodiments, the truncated USP7-binding domain comprises 1-5, 1-4, 1-3, 1-2, or 1 amino acid. In some embodiments, the modified HSV ICP0 does not comprise a RING finger domain. In some embodiments, the modified HSV ICP0 does not comprise a USP7-binding domain. In some embodiments, the modified HSV ICP0 does not bind USP7 in a cell.

In some embodiments, an mRNA of an HSV vaccine of the present disclosure encodes an HSV-1 ICP4. In other embodiments, an mRNA of an HSV vaccine of the present disclosure encodes an HSV-2 ICP4. In some embodiments, a wild-type HSV-1 ICP4 comprises the amino acid sequence of SEQ ID NO: 119. In some embodiments, a wild-type HSV-2 ICP4 comprises the amino acid sequence of SEQ ID NO: 120.

In some embodiments, an mRNA of an HSV vaccine of the present disclosure encodes an HSV ICP4 with a truncation in one or more of a nuclear localization signal, RING finger domain, or DNA-binding domain. An HSV ICP4 with a truncation in a given domain refers to an HSV ICP4 that lacks one or more amino acids that are present in that domain in wild-type HSV ICP4. For example, an HSV-2 ICP4 having the amino acid sequence of SEQ ID NO: 120 (Accession No. P90493) comprises a nuclear localization signal at amino acids 751-834 and a DNA-binding domain at amino acids 319-547. See, e.g., Mullen et al., J Virol. 1994. 68(5):3250-3266. Thus, in SEQ ID NO: 120, a nuclear localization signal corresponds to amino acids 751-834, and so a modified ICP4 having a truncation in a nuclear localization signal relative to SEQ ID NO: 120 lacks one or more amino acids corresponding to amino acids 751-834 of SEQ ID NO: 120. In SEQ ID NO: 120, a DNA-binding domain corresponds to amino acids 319-547, and so a modified ICP4 having a truncation in a DNA-binding domain relative to SEQ ID NO: 120 lacks one or more amino acids corresponding to amino acids 319-547 of SEQ ID NO: 120.

Some portions of ICP4 that are shortened or removed by truncation (e.g., DNA-binding domains and/or nuclear localization signals) are located internally on a wild-type HSV ICP4, and so truncations in these domains are internal truncations. An HSV ICP4 comprising a truncation in one or more of these domains or signals is truncated internally, such that it lacks one or more amino acids that is present in the domain of a wild-type ICP4 sequence, but comprises one or more amino acids that flank the deleted amino acid(s) in the wild-type ICP4 sequence. Additionally, an HSV ICP4 encoded by an mRNA of a vaccine of the present disclosure may comprise a truncated C-terminus and/or a truncated N-terminus. In some embodiments, a modified HSV ICP4 lacks an amino acid sequence comprising 1-400, 1-380, 1-360, 1-340, 1-320, 1-300, 1-280, 1-260, 1-240, 1-220, 1-200, 1-190, 1-180, 1-170, 1-160, 1-150, 1-140, 1-130, 1-120, 1-110, 1-100, 1-90, 1-80, 1-70, 1-60, 1-50, 1-40, 1-30, 1-25, 1-20, 1-10, or 1-5, 10-400, 20-400, 30-400, 40-400, 50-400, 60-400, 70-400, 80-400, 90-400, 100-400, 110-400, 120-400, 130-400, 140-400, 150-400, 160-400, 170-400, 180-400, 190-400, 200-400, 220-400, 240-400, 260-400, 280-400, 300-400, 320-400, 340-400, 360-400, 380-400, 10-30, 30-50, 50-75, 75-100, 100-125, 125-150, 150-175, 175-200, 200-240, 240-280, 280-320, 320-360, or 360-400 amino acids that is present that is present at the C-terminus of a wild-type ICP4 amino acid sequence. In some embodiments, a modified HSV ICP4 lacks an amino acid sequence comprising 1-400, 1-380, 1-360, 1-340, 1-320, 1-300, 1-280, 1-260, 1-240, 1-220, 1-200, 1-190, 1-180, 1-170, 1-160, 1-150, 1-140, 1-130, 1-120, 1-110, 1-100, 1-90, 1-80, 1-70, 1-60, 1-50, 1-40, 1-30, 1-25, 1-20, 1-10, or 1-5, 10-400, 20-400, 30-400, 40-400, 50-400, 60-400, 70-400, 80-400, 90-400, 100-400, 110-400, 120-400, 130-400, 140-400, 150-400, 160-400, 170-400, 180-400, 190-400, 200-400, 220-400, 240-400, 260-400, 280-400, 300-400, 320-400, 340-400, 360-400, 380-400, 10-30, 30-50, 50-75, 75-100, 100-125, 125-150, 150-175, 175-200, 200-240, 240-280, 280-320, 320-360, or 360-400 amino acids that is present that is present at the N-terminus of a wild-type ICP4 amino acid sequence. In some embodiments, a modified HSV ICP4 lacks an amino acid sequence comprising 1-400, 1-380, 1-360, 1-340, 1-320, 1-300, 1-280, 1-260, 1-240, 1-220, 1-200, 1-190, 1-180, 1-170, 1-160, 1-150, 1-140, 1-130, 1-120, 1-110, 1-100, 1-90, 1-80, 1-70, 1-60, 1-50, 1-40, 1-30, 1-25, 1-20, 1-10, or 1-5, 10-400, 20-400, 30-400, 40-400, 50-400, 60-400, 70-400, 80-400, 90-400, 100-400, 110-400, 120-400, 130-400, 140-400, 150-400, 160-400, 170-400, 180-400, 190-400, 200-400, 220-400, 240-400, 260-400, 280-400, 300-400, 320-400, 340-400, 360-400, 380-400, 10-30, 30-50, 50-75, 75-100, 100-125, 125-150, 150-175, 175-200, 200-240, 240-280, 280-320, 320-360, or 360-400 amino acids that is present between the N-terminal amino acid and C-terminal amino acid of a wild-type ICP4 amino acid sequence. In some embodiments, a modified HSV ICP4 lacks an amino acid sequence comprising 382 amino acids that is present at the N-terminus of a wild-type HSV ICP4. In some embodiments, a modified HSV ICP4 lacks an amino acid sequence comprising 75 amino acids that is present at the C-terminus of a wild-type HSV ICP4. In some embodiments, a modified HSV ICP4 lacks an amino acid sequence comprising 160-200 amino acids, and amino acid sequence comprising 1-10 amino acids, and an amino acid sequence comprising 11-30 amino acids, each of which are present between the N- and C-termini of a wild-type HSV ICP4. In some embodiments, a modified HSV ICP4 lacks an amino acid sequence comprising 181 amino acids, and amino acid sequence comprising 5 amino acids, and an amino acid sequence comprising 16 amino acids, each of which are present between the N- and C-termini of a wild-type HSV ICP4. In some embodiments, a modified HSV ICP4 lacks sequences corresponding to amino acids 1-382, 567-571, 614-629, 741-920, and 1244-1318 of a wild-type ICP4.

In some embodiments, a modified HSV ICP4 comprises one or more substitutions at residues corresponding to amino acids 1060-1070 of a wild-type HSV ICP4. In some embodiments, a modified HSV ICP4 comprises 1, 2, 3, 4, 5, 6, 7, 8, 9, 10, or 11 substitutions at residues corresponding to amino acids 1060-1070 of a wild-type HSV ICP4. In some embodiments, a modified HSV ICP4 comprises a substitution at a residue corresponding to amino acid 1064 of wild-type HSV ICP4. In some embodiments, a modified HSV ICP4 comprises a substitution at a residue corresponding to amino acid 1068 of wild-type HSV ICP4. In some embodiments, a modified HSV ICP4 comprises a substitution at a residue corresponding to amino acid 1069 of wild-type HSV ICP4. In some embodiments, a modified HSV ICP4 comprises substitutions at residues corresponding to amino acids 1064, 1068, and 1069. In some embodiments, one or more substitutions are substitutions with an aliphatic amino acid. In some embodiments, 1, 2, 3, 4, 5, 6, 7, 8, 9, 10, or 11 substitutions are substitutions with an aliphatic amino acid. In some embodiments, one or more substitutions are alanine substitutions. In some embodiments, 1, 2, 3, 4, 5, 6, 7, 8, 9, 10, or 11 substitutions are alanine substitutions. In some embodiments, a modified HSV ICP4 comprises a D1064A substitution relative to a wild-type HSV ICP4. In some embodiments, a modified HSV ICP4 comprises a D1068A substitution relative to a wild-type HSV ICP4. In some embodiments, a modified HSV ICP4 comprises a G1069A substitution relative to a wild-type HSV ICP4.

In some embodiments, the modified HSV ICP4 comprises a truncated nuclear localization signal relative to a wild-type HSV ICP4. In some embodiments, the modified HSV ICP4 comprises a truncated nuclear localization signal relative to a wild-type HSV ICP4. In some embodiments, the modified HSV ICP4 comprises a truncated nuclear localization signal comprising no more than 50%, no more than 40%, no more than 30%, no more than 25%, no more than 20%, no more than 15%, no more than 10%, or no more than 5% the length of the nuclear localization signal of the wild-type HSV ICP4. In some embodiments, the modified HSV ICP4 comprises a truncated nuclear localization signal comprising no more than 75, no more than 70, no more than 60, no more than 50, no more than 40, no more than 30, no more than 25, no more than 20, no more than 10, no more than 9, no more than 8, no more than 7, no more than 6, no more than 5, no more than 4, no more than 3, no more than 2, or no more than 1 amino acid. In some embodiments, the modified HSV ICP4 comprises a truncated nuclear localization signal comprising 1, 2, 3, 4, 5, 6, 7, 8, 9, 10, 11, 12, 13, 14, 15, 16, 17, 18, 19, 20, 21, 22, 23, 24, or 25 amino acids. In some embodiments, the truncated nuclear localization signal comprises 1-20, 1-15, 1-10, or 1-5 amino acids. In some embodiments, the truncated nuclear localization signal comprises 1-5, 1-4, 1-3, 1-2, or 1 amino acid. In some embodiments, the modified HSV ICP4 does not comprise a nuclear localization signal. In some embodiments, the modified HSV ICP4 is present in the cytoplasm following translation of the mRNA encoding the HSV ICP4. In some embodiments, the modified HSV ICP4 does not localize to the nucleus following translation of the mRNA encoding the HSV ICP4. In some embodiments, the modified HSV ICP4 does not comprise a nuclear localization signal. In some embodiments, the modified HSV ICP4 is present in the cytoplasm following translation of the mRNA encoding the HSV ICP4. In some embodiments, the modified HSV ICP4 does not localize to the nucleus following translation of the mRNA encoding the HSV ICP4.

In some embodiments, the modified HSV ICP4 comprises a truncated DNA-binding domain relative to a wild-type HSV ICP4. In some embodiments, the modified HSV ICP4 comprises a truncated DNA-binding domain relative to a wild-type HSV ICP4. In some embodiments, the modified HSV ICP4 comprises a DNA-binding domain comprising no more than 50%, no more than 40%, no more than 30%, no more than 25%, no more than 20%, no more than 15%, no more than 10%, or no more than 5% the length of the DNA-binding domain of the wild-type HSV ICP4. In some embodiments, the modified HSV ICP4 comprises a truncated DNA-binding domain comprising no more than 200, no more than 180, no more than 160, no more than 140, no more than 120, no more than 100, no more than 90, no more than 80, no more than 70, no more than 60, no more than 50, no more than 40, no more than 30, no more than 25, no more than 20, no more than 15, no more than 10, no more than 9, no more than 8, no more than 7, no more than 6, no more than 5, no more than 4, no more than 3, no more than 2, or no more than 1 amino acid. In some embodiments, the modified HSV ICP4 comprises a truncated DNA-binding domain comprising 1, 2, 3, 4, 5, 6, 7, 8, 9, 10, 11, 12, 13, 14, 15, 16, 17, 18, 19, 20, 21, 22, 23, 24, or 25 amino acids. In some embodiments, the truncated DNA-binding domain comprises 1-50, 1-40, 1-30, 1-25, 1-20, 1-15, 1-10, or 1-5 amino acids. In some embodiments, the truncated DNA-binding domain comprises 1-5, 1-4, 1-3, 1-2, or 1 amino acid. In some embodiments, the modified HSV ICP4 does not comprise a DNA-binding domain. In some embodiments, the modified HSV ICP4 does not comprise a DNA-binding domain. In some embodiments, the modified HSV ICP4 does not bind DNA in a cell.

In some embodiments, a modified ICP0 or ICP4 comprises a linker. The linker may be a 2A or GS linker described herein in the section entitled “Linkers and Cleavable Peptides.” Alternatively, the linker may be another linker known in the art. A linker of a modified ICP0 or ICP4 may be present in place of an internal truncation relative to a wild-type sequence of the ICP0 or ICP4 (i.e., the amino acid sequence of the modified ICP0 or ICP4 comprises deletion of one or more amino acids, relative to the wild-type sequence, and insertion of the linker at the position previously occupied by the deleted amino acids). In some embodiments, the linker comprises 2-10 glycine residues. In some embodiments, the linker comprises 2, 3, 4, 5, 6, 7, 8, 9, 10, 11, 12, 13, 14, 15, 16, 17, 18, 19, or 20 glycine residues. In some embodiments, the linker comprises 2-20, 2-15, 2-10, 2-5, 2-3, 3-5, 5-7, 7-10, 10-15, 15-20, 3-10, 4-8, or 5-6 glycine residues. In some embodiments, the linker comprises 5-6 glycine residues. In some embodiments, the linker comprises the amino acid sequence GGGSGGG (SEQ ID NO: 78). In some embodiments, the modified ICP0 or ICP4 comprises a linker in place of one or more internal truncation from the wild-type sequence of the ICP0 or ICP4. In some embodiments, the modified ICP0 or ICP4 comprises a linker in place of each internal truncation relative to the wild-type ICP0 or ICP4, respectively. The linkers connecting portions of the modified ICP0 or ICP4 may comprise the same amino acid sequence (e.g., each portion is connected by a linker having the amino acid sequence GGS). In other embodiments, linkers connecting different pairs of portions of the modified ICP0 or ICP4 may comprise different amino acid sequences (e.g., a first and second portion are connected by a linker having the amino acid sequence GGG, and a second and third portion are connected by a linker having the amino acid sequence GGS). Linkers connecting different pairs of portions may be the same length, or different lengths.

Vaccines of the present disclosure are useful for generating HSV-specific antibodies and T cells which, in addition to prophylactically preventing HSV infection in subjects not yet exposed to HSV, are useful for preventing latent HSV reactivation or reducing the duration of an HSV outbreak in subjects previously infected with HSV.

In some embodiments, the vaccines provide herein further comprise RNA (e.g., mRNA) having an open reading frame that encodes HSV glycoprotein E (gE). Antibodies specific to HSV gE prevent HSV virions from sequestering other circulating antibodies, allowing neutralizing HSV-specific antibodies to more efficiently neutralize HSV particles.

The genome of Herpes Simplex Viruses (HSV-1 and HSV-2) contains about 85 open reading frames, such that HSV can generate at least 85 unique proteins. These genes encode 4 major classes of proteins: (1) those associated with the outermost external lipid bilayer of HSV (the envelope), (2) the internal protein coat (the capsid), (3) an intermediate complex connecting the envelope with the capsid coat (the tegument), and (4) proteins responsible for replication and infection.

Examples of envelope proteins include UL1 (gL), UL10 (gM), UL20, UL22, UL27 (gB), UL43, UL44 (gC), UL45, UL49A, UL53 (gK), US4 (gG), US5 (gJ), US6 (gD), US7 (gI), US8 (gE), and US10. Examples of capsid proteins include UL6, UL18, UL19, UL35, and UL38. Tegument proteins include UL11, UL13, UL21, UL36, UL37, UL41, UL45, UL46, UL47, UL48, UL49, US9, and US10. Other HSV proteins include UL2, UL3, UL4, UL5, UL7, UL8, UL9, UL12, UL14, UL15, UL16, UL17, UL23, UL24, UL25, UL26, UL26.5, UL28, UL29, UL30, UL31, UL32, UL33, UL34, UL39, UL40, UL42, UL50, UL51, UL52, UL54, UL55, UL56, US1, US2, US3, US81, US11, US12, ICP0, and ICP4.

Since the envelope (most external portion of an HSV particle) is the first to encounter target cells, the present disclosure encompasses antigenic polypeptides associated with the envelope as immunogenic agents. In brief, surface and membrane proteins-glycoprotein D (gD), glycoprotein B (gB), glycoprotein C (gC), glycoprotein H (gH), glycoprotein L (gL) may be used as HSV vaccine antigens.

In epithelial cells, the heterodimer glycoprotein E/glycoprotein I (gE/gI) is required for the cell-to-cell spread of the virus, by sorting nascent virions to cell junctions. Once the virus reaches the cell junctions, virus particles can spread to adjacent cells extremely rapidly through interactions with cellular receptors that accumulate at these junctions. By similarity, it is implicated in basolateral spread in polarized cells. In neuronal cells, gE/gI is essential for the anterograde spread of the infection throughout the host nervous system. Together with US9, the heterodimer gE/gI is involved in the sorting and transport of viral structural components toward axon tips. The heterodimer gE/gI serves as a receptor for the Fc part of host IgG. Dissociation of gE/gI from IgG occurs at acidic pH, thus may be involved in anti-HSV antibodies bipolar bridging, followed by intracellular endocytosis and degradation, thereby interfering with host IgG-mediated immune responses. gE/gI interacts (via C-terminus) with VP22 tegument protein; this interaction is necessary for the recruitment of VP22 to the Golgi and its packaging into virions.

In some embodiments, HSV vaccines of the present disclosure comprise one or more RNAs (e.g., mRNAs) encoding HSV (HSV-1 or HSV-2) glycoproteins B, C, and D. In some embodiments, the HSV vaccines further comprise one or more RNAs encoding HSV (HSV-1 or HSV-2) glycoprotein E and intracellular protein 0 (ICP0).

In some embodiments, HSV vaccines comprise an RNA (e.g., mRNA) encoding an HSV (HSV-1 or HSV-2) protein having at least 95%, at least 96%, at least 97%, at least 98% or at least 99% identity with HSV (HSV-1 or HSV-2) glycoprotein B and has HSV (HSV-1 or HSV-2) glycoprotein B activity.

In some embodiments, HSV vaccines comprise an RNA (e.g., mRNA) encoding an HSV (HSV-1 or HSV-2) protein having at least 95%, at least 96%, at least 97%, at least 98% or at least 99% identity with HSV (HSV-1 or HSV-2) glycoprotein C and has HSV (HSV-1 or HSV-2) glycoprotein C activity.

In some embodiments, HSV vaccines comprise an RNA (e.g., mRNA) encoding an HSV (HSV-1 or HSV-2) protein having at least 95%, at least 96%, at least 97%, at least 98% or at least 99% identity with HSV (HSV-1 or HSV-2) glycoprotein D and has HSV (HSV-1 or HSV-2) glycoprotein D activity.

In some embodiments, HSV vaccines comprise an RNA (e.g., mRNA) encoding an HSV (HSV-1 or HSV-2) protein having at least 95%, at least 96%, at least 97%, at least 98% or at least 99% identity with HSV (HSV-1 or HSV-2) glycoprotein E and has HSV (HSV-1 or HSV-2) glycoprotein E activity.

In some embodiments, HSV vaccines comprise an RNA (e.g., mRNA) encoding an HSV (HSV-1 or HSV-2) protein having at least 95%, at least 96%, at least 97%, at least 98% or at least 99% identity with HSV (HSV-1 or HSV-2) intracellular protein 0 and has HSV (HSV-1 or HSV-2) intracellular protein 0 activity.

Non-limiting examples of HSV proteins of the present disclosure are provided in Table 2.

HSV RNA (e.g., mRNA) vaccines, as provided herein may be used to induce a balanced immune response, comprising both cellular and humoral immunity, without many of the risks associated with DNA vaccination.

The RNA (e.g., mRNA) of the present disclosure encode a HSV protein of interest, intended to raise an immune response to HSV infection. Thus, the HSV proteins of the present disclosure are antigenic, i.e., they are antigens. Antigenicity is the ability to be specifically recognized by antibodies generated as a result of an immune response to a given substance, such as a HSV protein of the present disclosure. Thus, an antigens is a protein capable of inducing an immune response (e.g., causing an immune system to produce antibodies against the antigen). In some embodiments, an antigen is an immunogen. Immunogenicity refers to the ability of a substance to induce cellular and humoral immune responses. The compositions of the present disclosure do not comprise antigens per se, but rather comprise RNA (e.g., mRNA) that have an open reading frame encoding a protein antigen (referred to herein simply as a “HSV protein”) that once delivered to subject is expressed by cells in the subject. Delivery of the RNA (e.g., mRNA) is achieved by formulating the RNA in appropriate carriers or delivery vehicles (e.g., lipid nanoparticles) such that upon administration to cells, tissues or subjects, the RNA is taken up by cells which, in turn, express the protein(s) encoded by the RNA.

It should be understood that the term “protein” encompasses peptides and the term “antigen” encompasses antigenic fragments.

The vaccines of the present disclosure provide a unique advantage over traditional protein-based vaccination approaches in which protein antigens are purified or produced in vitro, e.g., recombinant protein production technologies. The vaccines of the present disclosure comprise RNA (e.g., mRNA) encoding the desired HSV protein antigen(s), which when introduced into the body, i.e., administered to a mammalian subject (for example a human) in vivo, cause the cells of the body to express the desired antigen(s). In order to facilitate delivery of the RNA (e.g., mRNA) to the cells of the body, the RNA is encapsulated in a lipid nanoparticle (LNP). Upon delivery and uptake by cells of the body, the RNA is translated in the cytosol and protein antigens are generated by the host cell machinery. The proteins are presented and elicit an adaptive humoral and cellular immune response. Neutralizing antibodies are directed against the expressed proteins, and hence the proteins are considered relevant target antigens for vaccine development.

Many proteins have a quaternary or three-dimensional structure, which includes more than one polypeptide or several polypeptide chains that associate into an oligomeric molecule. As used herein the term “subunit” refers to a single protein molecule, for example, a polypeptide or polypeptide chain resulting from processing of a nascent protein molecule, which subunit assembles (or “coassembles”) with other protein molecules (e.g., subunits or chains) to form a protein complex. Proteins can have a relatively small number of subunits and therefore be described as “oligomeric” or can consist of a large number of subunits and therefore be described as “multimeric”. The subunits of an oligomeric or multimeric protein may be identical, homologous or totally dissimilar and dedicated to disparate tasks.

Proteins or protein subunits can further comprise domains. As used herein, the term “domain” refers to a distinct functional and/or structural unit within a protein. Typically, a “domain” is responsible for a particular function or interaction, contributing to the overall role of a protein. Domains can exist in a variety of biological contexts. Similar domains (i.e., domains sharing structural, functional and/or sequence homology) can exist within a single protein or can exist within distinct proteins having similar or different functions. A protein domain is often a conserved part of a given protein tertiary structure or sequence that can function and exist independently of the rest of the protein or subunit thereof.

As used herein, the term “antigen” is distinct from the term “epitope,” which is a substructure of an antigen. An epitope of a part of an antigen to which an antibody attaches. An epitope may be a peptide, for example, a 7-10 amino acid peptide, or a carbohydrate structure. The art describes protein antigens that are delivered to subjects or immune cells in isolated form, e.g., isolated proteins, however, the design, testing, validation, and production of protein antigens can be costly and time-consuming, especially when producing proteins at large scale. By contrast, mRNA technology is amenable to rapid design and testing of mRNA encoding a variety of antigens. Moreover, rapid production of mRNA coupled with formulation in appropriate delivery vehicles (e.g., lipid nanoparticles), can proceed quickly and can rapidly produce mRNA vaccines at large scale. Potential benefit also arises from the fact that antigens encoded by the mRNAs of the present disclosure are expressed by the cells of the subject, e.g., are expressed by the human body, and thus the subject, e.g., the human body, serves as the “factory” to produce the antigens which, in turn, elicits the desired immune response.

The vaccines, as provided herein, may include an RNA (e.g., mRNA) or multiple RNAs encoding two or more antigens of the same or different HSV strains. Also provided herein are combination vaccines that include RNA (e.g., mRNA) encoding one or more HSV antigens and one or more antigen(s) of a different organism. Thus, the vaccines of the present disclosure may be combination vaccines that target one or more antigens of the same strain/species, or one or more antigens of different strains/species, e.g., antigens that induce immunity to organisms that are found in the same geographic areas where the risk of HSV infection is high or organisms to which an individual is likely to be exposed to when exposed to HSV.

The vaccines, as provided herein, may include multiple RNAs encoding different antigens.

In some embodiments, the molar ratio of (a) an mRNA encoding an HSV ICP0 to (b) an mRNA encoding an HSV gB is no more than 0.8:1. In some embodiments, the molar ratio is about 0.65:1.

In some embodiments, the molar ratio of (a) an mRNA encoding an HSV ICP0 to (b) an mRNA encoding an HSV gC is no more than 1.1:1. In some embodiments, the molar ratio is about 1.0:1.

In some embodiments, the molar ratio of (a) an mRNA encoding an HSV ICP0 to (b) an mRNA encoding an HSV gD is no more than 1.3:1. In some embodiments, the molar ratio is about 1.2:1.

In some embodiments, the molar ratio of (a) an mRNA encoding an HSV ICP4 to (b) an mRNA encoding an HSV gB is no more than 1.0:1. In some embodiments, the molar ratio is about 0.9:1.

In some embodiments, the molar ratio of (a) an mRNA encoding an HSV ICP4 to (b) an mRNA encoding an HSV gC is no more than 1.5:1. In some embodiments, the molar ratio is about 1.4:1.

In some embodiments, the molar ratio of (a) an mRNA encoding an HSV ICP4 to (b) an mRNA encoding an HSV gD is no more than 1.8:1. In some embodiments, the molar ratio is about 1.6:1.

In some embodiments the molar ratio of (a) an mRNA encoding an HSV ICP0 and an mRNA encoding an HSV ICP4, to (b) an mRNA encoding an HSV gB is no more than 1.6:1. In some embodiments, the molar ratio is about 1.5:1.

In some embodiments the molar ratio of (a) an mRNA encoding an HSV ICP0 and an mRNA encoding an HSV ICP4, to (b) an mRNA encoding an HSV gC is no more than 2.5:1. In some embodiments, the molar ratio is about 2.4:1.

In some embodiments the molar ratio of (a) an mRNA encoding an HSV ICP0 and an mRNA encoding an HSV ICP4, to (b) an mRNA encoding an HSV gD is no more than 2.9:1. In some embodiments, the molar ratio is about 2.8:1.

In some embodiments, the molar ratio of (a) mRNAs encoding HSV ICP0 and ICP4 to (b) mRNAs encoding HSV gB, gC, and gD is no more than 0.8:1. In some embodiments, the molar ratio is about 0.7:1.

### Variants of Wild-Type Herpes Simplex Virus Proteins

In some embodiments, the compositions of the present disclosure include RNA (e.g., mRNA) that encodes a HSV protein variant. Protein variants are proteins (including full length proteins and peptides) that differ in their amino acid sequence relative to a wild-type, native, or reference amino acid sequence. A protein variant may possess one or more substitutions, deletions, and/or insertions at certain positions within its amino acid sequence, as compared to a wild-type, native, or reference amino acid sequence. Ordinarily, protein variants have at least 50% identity to a wild-type, native or reference sequence. In some embodiments, a protein variant has at least 60%, at least 70%, at least 75%, at least 80%, at least 85%, at least 90%, or at least 95% identity to a wild-type, native, or reference sequence.

A protein variant encoded by an RNA (e.g., mRNA) of the disclosure may contain amino acid changes that confer any of a number of desirable properties, for example, that enhance its immunogenicity, enhance its expression, and/or improve its stability or PK/PD properties in a subject. Protein variants can be made using routine mutagenesis techniques and assayed as appropriate to determine whether they possess the desired property. Assays to determine expression levels and immunogenicity of proteins, including protein variants, are well known in the art. Similarly, PK/PD properties of a protein variant can be measured using art recognized techniques, for example, by determining expression of the protein variant in a vaccinated subject over time and/or by looking at the durability of an induced immune response. The stability of a protein variant encoded by an RNA (e.g., mRNA) may be measured by assaying thermal stability or stability upon urea denaturation or may be measured using in silico prediction, for example. Methods for such experiments and in silico determinations are known in the art. Other methods for determining protein variant expression levels, immunogenicity and/or PK/PD properties of a protein variant may be used.

In some embodiments, an RNA (e.g., mRNA) comprises an open reading frame that comprises a nucleotide sequence of any one of the sequences provided herein or comprises a nucleotide sequence that has at least 50%, at least 60%, at least 70%, at least 75%, at least 80%, at least 85%, at least 90%, or at least 95% identity to a nucleotide sequence of any one of the sequences provided herein. See, e.g., SEQ ID NOs: 1-34, which are reproduced below in Table 1.

In some embodiments, an RNA (e.g., mRNA) comprises an open reading frame that encodes a protein comprising an amino acid sequence of any one of the sequences provided herein or comprises an amino acid sequence that has at least 50%, at least 60%, at least 70%, at least 75%, at least 80%, at least 85%, at least 90%, or at least 95% identity to the amino acid sequence of any one of the sequences provided herein. See, e.g., SEQ ID NOs: 35-68, which are reproduced below in Table 2.

“Identity” refers to a relationship between two or among three or more sequences (e.g., amino acid sequences or nucleotide sequences) as determined by comparing the sequences to each other. Identity also refers to the degree of sequence relatedness between or among sequences as determined by the number of matches between or among strings of amino acids (polypeptides) or strings of nucleotides (polynucleotides). Identity is a measure of the percent of identical matches between the smaller of two or more sequences with gap alignments (if any) addressed by a particular mathematical model or computer program (e.g., “algorithms”). Identity of related polypeptides and polynucleotides can be readily calculated by known methods. “Percent (%) identity” as it applies to polypeptide or polynucleotide sequences is defined as the percentage of residues (amino acid or nucleic acid residues) in the candidate (first) polypeptide or polynucleotide sequence that are identical with the residues in a second polypeptide or polynucleotide sequence after aligning the sequences and introducing gaps, if necessary, to achieve the maximum percent identity.

Methods and computer programs for the alignment are well known in the art. It is understood that identity depends on a calculation of percent identity but may differ in value due to gaps and penalties introduced in the calculation. Generally, variants of a particular polynucleotide or polypeptide have at least 90%, 91%, 92%, 93%, 94%, 95%, 96%, 97%, 98%, 99% but less than 100% sequence identity to that particular wild-type, native, or reference sequence as determined by sequence alignment programs and parameters described herein and known to those skilled in the art. Such tools for alignment include but are not limited to those of the BLAST suite (Altschul, S. F., et al. Nucleic Acids Res. 1997; 25:3389-3402); and those based on the Smith-Waterman algorithm (Smith, T. F. & Waterman, M. S. J. Mol. Biol. 1981; 147:195-197). A general global alignment technique based on dynamic programming is the Needleman-Wunsch algorithm (Needleman, S. B. & Wunsch, C. D. J. Mol. Biol. 1920; 48:443-453). A Fast Optimal Global Sequence Alignment Algorithm (FOGSAA) also has been developed that purportedly produces global alignment of nucleotide and protein sequences faster than other optimal global alignment methods, including the Needleman-Wunsch algorithm.

As such, polynucleotides and polypeptides containing substitutions, insertions and/or deletions (e.g., indels), and covalent modifications with respect to wild-type, native, or reference sequence, for example, the polypeptide (e.g., protein) sequences disclosed herein, are included within the scope of this disclosure. For example, sequence tags or amino acids, such as one or more lysine(s), can be added to polypeptide sequences (e.g., at the N-terminal and/or C-terminal end). Sequence tags can be used for peptide detection, purification and/or localization. Lysines can be used to increase peptide solubility or to allow for biotinylation. Alternatively, amino acid residues located at the N-terminal and/or C-terminal regions of the amino acid sequence of a protein may optionally be deleted providing for truncated sequences. Certain amino acids (e.g., C-terminal or N-terminal amino acids) may be deleted depending on the use of the sequence, as for example, expression of the sequence as part of a larger sequence that is soluble or linked to a solid support. In some embodiments, sequences for (or encoding) signal sequences, termination sequences, transmembrane domains, linkers, multimerization domains (e.g., foldon regions) and the like are substituted with alternative sequences that achieve the same or a similar function. In some embodiments, cavities in the core of proteins can be filled to improve stability, e.g., by introducing larger amino acids. In other embodiments, buried hydrogen bond networks are replaced with hydrophobic resides to improve stability. In yet other embodiments, glycosylation sites are removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA) vaccine.

As recognized by those skilled in the art, protein fragments, functional protein domains, and homologous proteins are also considered to be within the scope of HSV proteins provided herein. For example, provided herein is any protein fragment of (meaning a polypeptide sequence at least one amino acid residue shorter than but otherwise identical to) a wild-type, native, or reference sequence, provided that the fragment is immunogenic and confers a protective immune response to HSV. In addition to protein variants that are identical to the wild-type, native, or reference protein but are truncated, in some embodiments, a protein includes 2, 3, 4, 5, 6, 7, 8, 9, 10, or more mutations (e.g., substitutions, insertions and/or deletions), as shown in any of the sequences provided or referenced herein. Protein variants can range in length from about 4, 6, or 8 amino acids to full length proteins.

Non-limiting examples of HSV protein variants and nucleotide sequences encoding the HSV protein variants are provided in Table 1 and Table 2.

TABLE 1
Nucleic acid sequences encoding exemplary HSV antigens

| | | SEQ |
|---|---|---|
| | | ID |
| Protein | Sequence | NO. |
| gB WT | AUGCGGGGUGGCGGACUGGUGUGCGCCCUGGUGGUCGGUGCACUUGUGGCCGCUGUGG | 1 |
| | CUAGCGCUGCACCAGCCGCACCCAGAGCCAGCGGAGGUGUGGCCGCGACCGUUGCAGC | |
| | CAACGGAGGCCCGGCUUCUCAGCCUCCUCCUGUGCCCAGCCCCGCUACCACCAAGGCC | |
| | CGUAAGCGGAAGACCAAGAAGCCUCCCAAGCGGCCCGAGGCUACACCACCACCCGACG | |
| | CCAACGCAACUGUUGCUGCUGGCCACGCCACCCUGAGGGCCCACCUGCGGGAGAUCAA | |
| | GGUGGAGAACGCCGACGCCCAGUUCUACGUGUGUCCACCUCCAACCGGCGCCACCGUG | |
| | GUACAGUUCGAGCAACCUCGCCGGUGCCCCACUCGACCCGAGGGCCAGAACUACACCG | |
| | AGGGCAUCGCCGUGGUGUUCAAGGAGAACAUCGCCCCAUACAAGUUCAAGGCCACCAU | |
| | GUACUACAAGGACGUGACCGUGAGCCAGGUGUGGUUCGGCCACCGGUACAGCCAGUUC | |
| | AUGGGCAUCUUCGAGGAUCGGGCCCCAGUGCCCUUCGAGGAGGUGAUCGACAAGAUCA | |
| | ACGCCAAGGGCGUGUGCCGGAGCACCGCCAAGUACGUGCGGAACAACAUGGAGACCAC | |
| | CGCCUUCCACCGGGACGACCACGAGACCGACAUGGAGCUGAAGCCCGCCAAGGUGGCC | |
| | ACACGGACAAGCCGGGGCUGGCACACCACCGACCUGAAGUACAACCCAAGCCGCGUGG | |
| | AGGCGUUCCACCGGUACGGCACCACCGUGAACUGCAUCGUGGAGGAGGUGGACGCCCG | |
| | GAGCGUGUACCCCUACGACGAAUUCGUGCUGGCCACCGGCGACUUCGUGUACAUGAGC | |
| | CCCUUCUACGGCUACCGGGAGGGCAGCCACACCGAGCACACCAGCUACGCCGCCGACC | |
| | GGUUCAAGCAGGUGGACGGCUUCUACGCCCGGGACCUGACCACUAAAGCCCGGGCCAC | |
| | CUCACCAACCACCCGGAACCUGCUGACCACCCCAAAGUUCACCGUGGCCUGGGACUGG | |
| | GUGCCCAAGCGACCCGCCGUGUGCACCAUGACCAAGUGGCAGGAAGUGGACGAGAUGC | |
| | UGCGGGCCGAAUACGGCGGCAGCUUCCGGUUCAGCAGCGACGCCAUCAGCACCACCUU | |
| | CACCACCAACCUGACCCAGUACAGCCUGAGCCGGGUGGAUCUGGGCGACUGCAUCGGC | |
| | AGAGACGCUCGCGAGGCCAUCGACCGGAUGUUCGCCCGGAAGUACAACGCCACCCACA | |
| | UCAAGGUGGGCCAGCCCCAGUACUACCUCGCCACCGGCGGCUUCCUGAUCGCCUACCA | |
| | GCCCUUGCUGAGCAACACCCUGGCCGAGCUGUACGUGCGGGAGUACAUGCGGGAGCAG | |
| | GACCGGAAGCCCCGGAACGCCACACCCGCCCCUCUGAGAGAAGCCCCUUCCGCCAACG | |
| | CCAGCGUGGAGCGGAUCAAGACCACCAGCAGCAUCGAGUUCGCCCGGCUGCAGUUCAC | |
| | CUACAACCACAUCCAGCGGCACGUGAACGACAUGCUGGGUCGGAUCGCCGUGGCUUGG | |
| | UGCGAGCUGCAGAACCACGAGCUGACCCUGUGGAACGAGGCCCGGAAGCUGAACCCCA | |
| | ACGCCAUCGCCAGCGCUACUGUGGGCCGGAGGGUAAGCGCUCGGAUGCUGGGCGACGU | |
| | GAUGGCCGUGAGCACGUGUGUGCCCGUGGCCCCAGACAACGUGAUCGUGCAGAACAGC | |
| | AUGCGGGUGAGUAGCCGGCCUGGAACCUGCUACAGCCGGCCCCUGGUGUCUUUCCGGU | |
| | ACGAGGACCAGGGUCCCCUGAUCGAGGGCCAGCUGGGCGAGAACAACGAGCUGCGGCU | |
| | GACUCGCGACGCUCUGGAGCCCUGCACCGUGGGCCACAGACGGUACUUCAUCUUCGGC | |
| | GGCGGCUACGUGUACUUCGAGGAGUACGCCUACAGCCACCAGCUGAGCCGGGCCGACG | |
| | UGACCACCGUGAGCACCUUCAUCGACCUGAACAUCACCAUGCUGGAGGACCACGAGUU | |
| | CGUGCCCCUGGAGGUGUACACCCGGCACGAGAUCAAGGACAGCGGCCUGCUGGAUUAC | |
| | ACCGAGGUGCAGCGGCGGAACCAGCUGCACGACCUGCGGUUCGCCGACAUCGACACCG | |
| | UGAUACGGGCCGACGCAAACGCCGCCAUGUUCGCCGGCCUGUGCGCCUUCUUCGAGGG | |
| | CAUGGGCGACCUGGGCAGAGCCGUGGGCAAGGUGGUGAUGGGCGUGGUGGGCGGAGUG | |
| | GUAAGCGCCGUGAGCGGCGUGAGCAGCUUCAUGAGCAACCCCUUCGGUGCCCUGGCAG | |
| | UGGGCCUGCUGGUGCUUGCUGGCCUGGUCGCUGCCUUCUUCGCCUUCCGGUACGUGCU | |
| | ACAGCUGCAGCGGAACCCCAUGAAGGCCCUGUACCCUCUGACCACCAAGGAGCUGAAG | |
| | ACCUCAGACCCUGGCGGCGUUGGAGGAGAGGGUGAGGAGGGAGCAGAGGGCGGAGGCU | |
| | UCGACGAGGCCAAGCUGGCUGAGGCCCGGGAGAUGAUCCGGUACAUGGCCCUGGUGAG | |
| | CGCCAUGGAGCGGACCGAGCACAAGGCCAGAAAGAAGGGCACCAGCGCCCUGCUGAGC | |
| | AGCAAGGUGACCAACAUGGUGCUGCGGAAGCGGAACAAGGCCCGGUACUCACCCCUGC | |
| | ACAACGAGGACGAGGCCGGCGACGAAGACGAGCUG | |
| gB pf | AUGCGGGGUGGCGGACUGGUGUGCGCCCUGGUGGUCGGUGCACUUGUGGCCGCUGUGG | 2 |
| (H510P) | CUAGCGCUGCACCAGCCGCACCCAGAGCCAGCGGAGGUGUGGCCGCGACCGUUGCAGC | |
| | CAACGGAGGCCCGGCUUCUCAGCCUCCUCCUGUGCCCAGCCCCGCUACCACCAAGGCC | |
| | CGUAAGCGGAAGACCAAGAAGCCUCCCAAGCGGCCCGAGGCUACACCACCACCCGACG | |
| | CCAACGCAACUGUUGCUGCUGGCCACGCCACCCUGAGGGCCCACCUGCGGGAGAUCAA | |
| | GGUGGAGAACGCCGACGCCCAGUUCUACGUGUGUCCACCUCCAACCGGCGCCACCGUG | |
| | GUACAGUUCGAGCAACCUCGCCGGUGCCCCACUCGACCCGAGGGCCAGAACUACACCG | |
| | AGGGCAUCGCCGUGGUGUUCAAGGAGAACAUCGCCCCAUACAAGUUCAAGGCCACCAU | |
| | GUACUACAAGGACGUGACCGUGAGCCAGGUGUGGUUCGGCCACCGGUACAGCCAGUUC | |
| | AUGGGCAUCUUCGAGGAUCGGGCCCCAGUGCCCUUCGAGGAGGUGAUCGACAAGAUCA | |
| | ACGCCAAGGGCGUGUGCCGGAGCACCGCCAAGUACGUGCGGAACAACAUGGAGACCAC | |
| | CGCCUUCCACCGGGACGACCACGAGACCGACAUGGAGCUGAAGCCCGCCAAGGUGGCC | |
| | ACACGGACAAGCCGGGGCUGGCACACCACCGACCUGAAGUACAACCCAAGCCGCGUGG | |
| | AGGCGUUCCACCGGUACGGCACCACCGUGAACUGCAUCGUGGAGGAGGUGGACGCCCG | |
| | GAGCGUGUACCCCUACGACGAAUUCGUGCUGGCCACCGGCGACUUCGUGUACAUGAGC | |
| | CCCUUCUACGGCUACCGGGAGGGCAGCCACACCGAGCACACCAGCUACGCCGCCGACC | |
| | GGUUCAAGCAGGUGGACGGCUUCUACGCCCGGGACCUGACCACUAAAGCCCGGGCCAC | |
| | CUCACCAACCACCCGGAACCUGCUGACCACCCCAAAGUUCACCGUGGCCUGGGACUGG | |
| | GUGCCCAAGCGACCCGCCGUGUGCACCAUGACCAAGUGGCAGGAAGUGGACGAGAUGC | |
| | UGCGGGCCGAAUACGGCGGCAGCUUCCGGUUCAGCAGCGACGCCAUCAGCACCACCUU | |
| | CACCACCAACCUGACCCAGUACAGCCUGAGCCGGGUGGAUCUGGGCGACUGCAUCGGC | |
| | AGAGACGCUCGCGAGGCCAUCGACCGGAUGUUCGCCCGGAAGUACAACGCCACCCACA | |
| | UCAAGGUGGGCCAGCCCCAGUACUACCUCGCCACCGGCGGCUUCCUGAUCGCCUACCA | |
| | GCCCUUGCUGAGCAACACCCUGGCCGAGCUGUACGUGCGGGAGUACAUGCGGGAGCAG | |
| | GACCGGAAGCCCCGGAACGCCACACCCGCCCCUCUGAGAGAAGCCCCUUCCGCCAACG | |
| | CCAGCGUGGAGCGGAUCAAGACCACCAGCAGCAUCGAGUUCGCCCGGCUGCAGUUCAC | |
| | CUACAACCACAUCCAGCGGCCUGUGAACGACAUGCUGGGUCGGAUCGCCGUGGCUUGG | |
| | UGCGAGCUGCAGAACCACGAGCUGACCCUGUGGAACGAGGCCCGGAAGCUGAACCCCA | |
| | ACGCCAUCGCCAGCGCUACUGUGGGCCGGAGGGUAAGCGCUCGGAUGCUGGGCGACGU | |
| | GAUGGCCGUGAGCACGUGUGUGCCCGUGGCCCCAGACAACGUGAUCGUGCAGAACAGC | |
| | AUGCGGGUGAGUAGCCGGCCUGGAACCUGCUACAGCCGGCCCCUGGUGUCUUUCCGGU | |
| | ACGAGGACCAGGGUCCCCUGAUCGAGGGCCAGCUGGGCGAGAACAACGAGCUGCGGCU | |
| | GACUCGCGACGCUCUGGAGCCCUGCACCGUGGGCCACAGACGGUACUUCAUCUUCGGC | |
| | GGCGGCUACGUGUACUUCGAGGAGUACGCCUACAGCCACCAGCUGAGCCGGGCCGACG | |
| | UGACCACCGUGAGCACCUUCAUCGACCUGAACAUCACCAUGCUGGAGGACCACGAGUU | |
| | CGUGCCCCUGGAGGUGUACACCCGGCACGAGAUCAAGGACAGCGGCCUGCUGGAUUAC | |
| | ACCGAGGUGCAGCGGCGGAACCAGCUGCACGACCUGCGGUUCGCCGACAUCGACACCG | |
| | UGAUACGGGCCGACGCAAACGCCGCCAUGUUCGCCGGCCUGUGCGCCUUCUUCGAGGG | |
| | CAUGGGCGACCUGGGCAGAGCCGUGGGCAAGGUGGUGAUGGGCGUGGUGGGCGGAGUG | |
| | GUAAGCGCCGUGAGCGGCGUGAGCAGCUUCAUGAGCAACCCCUUCGGUGCCCUGGCAG | |
| | UGGGCCUGCUGGUGCUUGCUGGCCUGGUCGCUGCCUUCUUCGCCUUCCGGUACGUGCU | |
| | ACAGCUGCAGCGGAACCCCAUGAAGGCCCUGUACCCUCUGACCACCAAGGAGCUGAAG | |
| | ACCUCAGACCCUGGCGGCGUUGGAGGAGAGGGUGAGGAGGGAGCAGAGGGCGGAGGCU | |
| | UCGACGAGGCCAAGCUGGCUGAGGCCCGGGAGAUGAUCCGGUACAUGGCCCUGGUGAG | |
| | CGCCAUGGAGCGGACCGAGCACAAGGCCAGAAAGAAGGGCACCAGCGCCCUGCUGAGC | |
| | AGCAAGGUGACCAACAUGGUGCUGCGGAAGCGGAACAAGGCCCGGUACUCACCCCUGC | |
| | ACAACGAGGACGAGGCCGGCGACGAAGACGAGCUG | |
| gC F327A | AUGGCACUUGGACGCGUCGGUCUGGCUGUCGGCCUGUGGGGCCUGCUGUGGGUGGGCG | 3 |
| | UGGUGGUGGUGCUGGCUAACGCCAGCCCCGGACGUACCAUCACCGUGGGGCCCAGAGG | |
| | CAACGCCAGUAACGCCGCGCCAUCAGCCAGCCCGAGAAACGCUAGUGCUCCCCGGACC | |
| | ACGCCAACUCCACCACAGCCCCGGAAGGCCACCAAGAGCAAGGCCAGCACCGCCAAGC | |
| | CCGCUCCACCUCCCAAGACCGGCCCACCCAAGACCAGCAGCGAGCCCGUGCGGUGCAA | |
| | CCGGCACGAUCCUCUGGCCCGGUACGGCUCACGGGUGCAGAUCCGGUGCCGGUUCCCC | |
| | AACAGCACCAGGACCGAGAGCCGGCUGCAGAUCUGGCGGUACGCCACCGCCACAGACG | |
| | CCGAGAUCGGCACCGCCCCAAGCCUGGAGGAGGUGAUGGUGAACGUGUCUGCUCCACC | |
| | UGGCGGCCAGCUGGUGUACGACAGCGCACCCAACCGGACCGAUCCCCACGUGAUCUGG | |
| | GCAGAAGGCGCUGGUCCUGGCGCUAGCCCUCGACUGUACAGCGUGGUCGGCCCACUGG | |
| | GCAGGCAGCGGCUGAUCAUCGAGGAGCUGACCCUGGAGACCCAGGGCAUGUACUACUG | |
| | GGUGUGGGGCCGGACAGAUCGGCCUAGCGCCUACGGGACCUGGGUGCGGGUUCGCGUG | |
| | UUCCGGCCACCUAGCCUGACCAUCCAUCCCCACGCCGUGCUGGAGGGCCAGCCCUUCA | |
| | AAGCCACCUGUACCGCCGCCACCUACUACCCAGGCAACCGGGCCGAGUUCGUGUGGUU | |
| | CGAGGACGGACGGCGCGUGUUCGACCCCGCCCAGAUCCACACCCAGACCCAGGAGAAC | |
| | CCCGACGGCUUCAGCACCGUGAGCACGGUGACCAGCGCUGCCGUUGGAGGCCAAGGCC | |
| | CUCCCAGAACCUUCACCUGCCAGCUGACCUGGCACCGGGACAGCGUGAGCGCUUCUCG | |
| | CCGGAACGCCAGCGGAACUGCCAGCGUGCUGCCACGGCCCACCAUCACCAUGGAGUUC | |
| | ACCGGCGACCACGCCGUGUGCACAGCCGGCUGCGUACCCGAGGGCGUGACCUUCGCCU | |
| | GGUUCCUGGGCGACGACAGCAGCCCCGCCGAGAAGGUGGCCGUGGCCAGCCAGACCAG | |
| | CUGUGGAAGACCCGGAACCGCCACCAUCCGGAGCACCCUGCCCGUGAGCUACGAGCAG | |
| | ACCGAGUACAUCUGUCGGCUGGCCGGCUACCCUGACGGCAUCCCCGUGUUGGAGCACC | |
| | ACGGCAGCCACCAGCCUCCUCCUCGGGAUCCCACCGAGCGGCAGGUGAUCAGGGCCGU | |
| | GGAGGGUGCAGGCAUCGGCGUGGCCGUGCUGGUGGCCGUAGUGCUAGCCGGCACCGCC | |
| | GUGGUAUACCUGACCCACGCCAGCAGCGUGCGCUACCGGAGACUGCGG | |
| gD WT* | AUGGGUCGGCUGACCAGCGGAGUUGGCACCGCCGCACUCCUGGUGGUGGCCGUAGGCC | 4 |
| | UGCGGGUGGUGUGCGCCAAGUACGCCCUGGCCGACCCAUCCCUGAAGAUGGCCGACCC | |
| | UAACCGGUUCCGGGGCAAGAAUCUGCCCGUGCUGGAUCAGCUGACCGAUCCUCCUGGC | |
| | GUGAAGCGGGUGUACCACAUCCAGCCCAGCCUGGAGGAUCCCUUCCAGCCACCCUCCA | |
| | UCCCCAUCACCGUUUACUACGCCGUGCUGGAGAGAGCCUGCCGCAGCGUGCUGCUGCA | |
| | CGCUCCAUCCGAGGCGCCCCAGAUCGUGCGGGGCGCAAGCGACGAGGCCCGGAAGCAC | |
| | ACCUACAACCUGACCAUCGCCUGGUACCGGAUGGGCGACAACUGCGCCAUCCCUAUCA | |
| | CCGUGAUGGAGUACACCGAGUGCCCCUACAACAAGAGCCUGGGAGUGUGCCCCAUCCG | |
| | GACCCAGCCUCGGUGGUCCUACUACGACAGCUUCAGCGCCGUGUCCGAGGACAACCUG | |
| | GGCUUCCUGAUGCACGCCCCUGCCUUCGAGACCGCCGGCACCUACCUGCGGCUGGUGA | |
| | AGAUCAACGACUGGACCGAGAUCACCCAGUUCAUCCUGGAGCACCGGGCAAGGGCCAG | |
| | CUGCAAGUACGCGCUGCCUCUGCGGAUCCCUCCCGCUGCUUGCCUGACCAGCAAGGCC | |
| | UACCAGCAGGGCGUGACCGUGGACAGCAUCGGCAUGCUGCCCAGGUUCAUCCCCGAGA | |
| | ACCAGCGCACCGUGGCCCUGUACAGCCUGAAGAUCGCAGGCUGGCACGGACCCAAGCC | |
| | UCCUUACACCUCCACCCUGCUGCCACCCGAGCUGAGCGACACCACCAACGCCACCCAG | |
| | CCCGAGCUGGUGCCCGAGGACCCCGAGGACUCCGCCCUGCUGGAGGAUCCGGCCGGCA | |
| | CCGUAAGCUCCCAGAUCCCACCCAACUGGCACAUCCCCAGCAUCCAGGACGUGGCCCC | |
| | ACAUCACGCGCCUGCCGCUCCAAGCAACCCCGGCCUGAUCAUUGGAGCUCUGGCCGGG | |
| | AGCACUCUGGCGGUGCUGGUGAUCGGCGGCAUCGCCUUCUGGGUGCGUCGGAGAGCCC | |
| | AGAUGGCCCCUAAGCGGCUGCGGCUGCCACACAUACGGGACGACGACGCGCCACCAUC | |
| | CCACCAGCCCCUGUUCUAC | |
| gE | AUGGCCAGAGGAGCCGGCCUGGUGUUCUUCGUGGGCGUGUGGGUGGUGUCCUGCCUGG | 5 |
| | CCGCAGCUCCCAGAACCAGCUGGAAGCGGGUGACCAGCGGCGAGGACGUGGUGUUACU | |
| | GCCAGCUCCAGCCGAGCGGACUCGGGCCCACAAGCUGCUUUGGGCAGCCGAGCCACUG | |
| | GACGCCUGCGGGCCAUUACGGCCUAGCUGGGUGGCUCUGUGGCCACCUAGACGGGUGC | |
| | UGGAGACCGUGGUGGACGCAGCCUGCAUGCGGGCACCAGAGCCCCUGGCCAUCGCCUA | |
| | CUCUCCACCCUUCCCAGCCGGCGACGAAGGCCUGUACAGCGAGCUGGCUUGGAGAGAC | |
| | CGGGUGGCCGUGGUGAACGAGAGCCUGGUGAUCUACGGCGCCCUGGAGACCGACAGCG | |
| | GCCUGUACACCCUGAGCGUGGUGGGCCUGAGCGACGAGGCCCGGCAAGUGGCCAGCGU | |
| | GGUGCUGGUGGUUGAGCCCGCCCCUGUGCCAACGCCCACUCCCGACGACUACGACGAG | |
| | GAGGACGACGCAGGCGUGAGCGAGCGGACCCCAGUGAGCGUGCCUCCACCCACUCCUC | |
| | CUCGGAGACCUCCCGUGGCUCCACCUACCCAUCCCCGGGUGAUCCCCGAGGUGAGCCA | |
| | CGUGCGGGGCGUGACCGUGCACAUGGAGACACCCGAGGCCAUCCUGUUCGCCCCUGGC | |
| | GAGACCUUCGGCACCAACGUGAGCAUCCACGCCAUAGCCCACGACGACGGCCCUUACG | |
| | CCAUGGACGUCGUGUGGAUGCGGUUCGACGUGCCCAGCAGCUGCGCCGAGAUGCGGAU | |
| | CUACGAGGCCUGCCUGUACCAUCCCCAGCUGCCCGAGUGCCUGAGCCCCGCUGACGCA | |
| | CCCUGCGCCGUGAGCAGCUGGGCCUACCGGCUGGCUGUGCGGAGCUACGCCGGGUGUA | |
| | GCCGGACAACGCCUCCGCCACGGUGCUUCGCCGAGGCCCGGAUGGAACCUGUUCCCGG | |
| | CCUGGCCUGGCUUGCUAGCACCGUGAACCUGGAGUUCCAGCACGCCAGCCCACAACAC | |
| | GCCGGCCUGUACCUGUGCGUGGUGUACGUGGACGACCACAUCCACGCCUGGGGCCACA | |
| | UGACCAUCAGCACCGCCGCCCAGUACCGGAACGCCGUGGUGGAGCAGCAUCUGCCCCA | |
| | GAGGCAGCCCGAGCCAGUGGAGCCCACGAGACCACACGUGCGGGCACCUCACCCUGCU | |
| | CCCAGCGCACGUGGGCCACUGCGGCUGGGAGCUGUGCUGGGCGCCGCUCUGCUGCUGG | |
| | CAGCCCUGGGACUGAGCGCCUGGGCCUGCAUGACCUGUUGGAGGAGGCGGUCUUGGCG | |
| | CGCAGUGAAGAGCCGGGCCAGUGCCACAGGCCCAACCUACAUCCGGGUGGCCGACAGC | |
| | GAGCUGUACGCCGACUGGAGCAGCGACAGCGAGGGCGAACGGGACGGCAGCCUGUGGC | |
| | AGGAUCCACCAGAGCGGCCUGACAGCCCCAGCACCAACGGCAGCGGCUUCGAGAUCCU | |
| | GAGCCCAACCGCCCCUAGCGUGUACCCUCACAGCGAGGGACGGAAGAGCAGACGGCCC | |
| | CUGACCACCUUCGGGUCUGGCAGCCCAGGCCGGAGACACAGCCAGGCCAGCUACCCCA | |
| | GCGUGCUGUGG | |
| sgE | AUGGCCAGAGGAGCCGGCCUGGUGUUCUUCGUGGGCGUGUGGGUGGUGUCCUGCCUGG | 6 |
| | CCGCAGCUCCCAGAACCAGCUGGAAGCGGGUGACCAGCGGCGAGGACGUGGUGUUACU | |
| | GCCAGCUCCAGCUGGCCCUGAGGAGCGGACUCGGGCCCACAAGCUGCUUUGGGCAGCC | |
| | GAGCCACUGGACGCCUGCGGGCCAUUACGGCCUAGCUGGGUGGCUCUGUGGCCACCUA | |
| | GACGGGUGCUGGAGACCGUGGUGGACGCAGCCUGCAUGCGGGCACCAGAGCCCCUGGC | |
| | CAUCGCCUACUCUCCACCCUUCCCAGCCGGCGACGAAGGCCUGUACAGCGAGCUGGCU | |
| | UGGAGAGACCGGGUGGCCGUGGUGAACGAGAGCCUGGUGAUCUACGGCGCCCUGGAGA | |
| | CCGACAGCGGCCUGUACACCCUGAGCGUGGUGGGCCUGAGCGACGAGGCCCGGCAAGU | |
| | GGCCAGCGUGGUGCUGGUGGUUGAGCCCGCCCCUGUGCCAACGCCCACUCCCGACGAC | |
| | UACGACGAGGAGGACGACGCAGGCGUGAGCGAGCGGACCCCAGUGAGCGUGCCUCCAC | |
| | CCACUCCUCCUCGGAGACCUCCCGUGGCUCCACCUACCCAUCCCCGGGUGAUCCCCGA | |
| | GGUGAGCCACGUGCGGGGCGUGACCGUGCACAUGGAGACACCCGAGGCCAUCCUGUUC | |
| | GCCCCUGGCGAGACCUUCGGCACCAACGUGAGCAUCCACGCCAUAGCCCACGACGACG | |
| | GCCCUUACGCCAUGGACGUCGUGUGGAUGCGGUUCGACGUGCCCAGCAGCUGCGCCGA | |
| | GAUGCGGAUCUACGAGGCCUGCCUGUACCAUCCCCAGCUGCCCGAGUGCCUGAGCCCC | |
| | GCUGACGCACCCUGCGCCGUGAGCAGCUGGGCCUACCGGCUGGCUGUGCGGAGCUACG | |
| | CCGGGUGUAGCCGGACAACGCCUCCGCCACGGUGCUUCGCCGAGGCCCGGAUGGAACC | |
| | UGUUCCCGGCCUGGCCUGGCUUGCUAGCACCGUGAACCUGGAGUUCCAGCACGCCAGC | |
| | CCACAACACGCCGGCCUGUACCUGUGCGUGGUGUACGUGGACGACCACAUCCACGCCU | |
| | GGGGCCACAUGACCAUCAGCACCGCCGCCCAGUACCGGAACGCCGUGGUGGAGCAGCA | |
| | UCUGCCCCAGAGGCAGCCCGAGCCAGUGGAGCCCACGAGACCACACGUGCGGGCACCU | |
| | CCACCUGCUCCCAGCGCACGUGGGCCACUGCGG | |
| gH | AUGGGCCCCGGUCUGUGGGUGGUGAUGGGCGUGCUGGUUGGCGUGGCUGGCGGACACG | 7 |
| | ACACCUACUGGACCGAGCAGAUCGACCCCUGGUUCCUGCACGGCCUGGGCUUGGCUCG | |
| | GACCUACUGGCGGGACACCAACACCGGACGGCUGUGGCUGCCCAACACCCCAGACGCC | |
| | AGCGAUCCCCAAAGAGGCCGGCUGGCUCCACCUGGCGAGCUGAACCUGACCACCGCCA | |
| | GCGUGCCCAUGCUGCGGUGGUACGCCGAGCGGUUCUGCUUCGUGCUGGUGACCACGGC | |
| | CGAGUUUCCACGGGACCCAGGCCAGCUGCUGUACAUCCCCAAGACCUACCUGCUGGGU | |
| | AGACCCCGGAACGCCAGCCUUCCCGAGCUGCCUGAGGCUGGCCCCACCUCACGACCAC | |
| | CCGCCGAGGUGACCCAGCUGAAGGGCCUGAGCCACAAUCCCGGAGCCAGCGCUCUGCU | |
| | GCGGAGCCGGGCUUGGGUGACCUUCGCCGCAGCUCCCGAUAGGGAGGGCCUGACAUUC | |
| | CCGAGAGGCGACGACGGGGCAACCGAAAGACACCCAGACGGCCGCAGAAACGCUCCAC | |
| | CUCCCGGACCACCAGCAGGUACGCCCCGGCAUCCCACCACCAACCUGAGCAUCGCCCA | |
| | CCUGCACAACGCCAGCGUGACUUGGCUGGCGGCCAGAGGUCUACUGCGGACGCCCGGC | |
| | AGAUACGUGUACCUGAGCCCCUCUGCCAGCACCUGGCCAGUGGGCGUGUGGACCACCG | |
| | GUGGCCUGGCCUUCGGUUGCGACGCCGCUCUCGUGCGAGCCCGGUACGGCAAGGGCUU | |
| | CAUGGGCCUGGUUAUCAGCAUGCGGGACAGUCCUCCCGCCGAGAUCAUCGUGGUGCCC | |
| | GCCGACAAGACCCUGGCCCGGGUUGGCAACCCCACCGACGAGAACGCACCCGCAGUGC | |
| | UGCCAGGGCCUCCAGCUGGCCCUCGGUACCGGGUGUUCGUGCUGGGAGCGCCAACCCC | |
| | AGCCGACAACGGCAGCGCGUUGGACGCCUUACGACGGGUGGCCGGCUAUCCCGAGGAG | |
| | AGCACCAACUACGCCCAGUACAUGAGCCGGGCCUACGCCGAGUUUCUGGGCGAGGACC | |
| | CAGGGAGCGGCACCGACGCAAGGCCCAGCCUGUUUUGGCGACUGGCAGGCCUCCUGGC | |
| | CAGCAGCGGCUUCGCCUUCGUGAACGCCGCACACGCGCACGACGCCAUCCGGCUGAGC | |
| | GACCUGCUGGGCUUCCUGGCCCACUCUCGGGUGCUCGCAGGCCUUGCUGCUCGGGGAG | |
| | CAGCCGGUUGUGCCGCCGACAGCGUGUUCCUGAACGUGAGCGUGCUUGACCCUGCGGC | |
| | CAGGCUGAGACUGGAGGCCCGGCUGGGACACCUGGUGGCCGCCAUCCUGGAGCGGGAG | |
| | CAGAGCCUGGCAGCCCACGCUCUGGGCUACCAGCUGGCCUUUGUUCUGGACAGCCCAG | |
| | CCGCCUACGGAGCCGUUGCCCCUUCCGCAGCCAGACUGAUCGACGCCCUGUACGCCGA | |
| | AUUCUUAGGUGGACGGGCCCUGACCGCACCAAUGGUGCGGCGGGCCCUGUUCUACGCC | |
| | ACCGCUGUCCUGCGGGCACCAUUCCUUGCCGGAGCCCCAUCUGCCGAGCAGCGGGAGC | |
| | GUGCCAGAAGGGGUCUGCUGAUCACCACCGCCCUGUGCACCAGCGACGUUGCUGCCGC | |
| | CACCCACGCUGAUUUACGAGCUGCCCUCGCACGGACCGACCACCAGAAGAACCUGUUC | |
| | UGGCUGCCCGACCACUUCUCUCCUUGCGCCGCGAGCCUACGGUUCGACCUGGCCGAAG | |
| | GCGGCUUCAUCCUGGACGCCCUCGCCAUGGCCACCCGGAGCGACAUCCCCGCCGACGU | |
| | GAUGGCCCAACAGACCCGGGGAGUGGCCAGUGCACUGACCCGGUGGGCCCACUACAAC | |
| | GCCCUGAUCCGGGCCUUCGUGCCCGAGGCCACCCACCAGUGCAGCGGCCCCAGCCACA | |
| | ACGCCGAGCCCCGGAUCCUGGUGCCCAUCACCCACAACGCUAGCUACGUGGUGACCCA | |
| | CACUCCCUUGCCCCGGGGAAUCGGCUACAAGCUGACCGGCGUGGACGUUCGGCGGCCC | |
| | CUGUUCAUCACCUACCUGACCGCCACCUGUGAGGGGCACGCCCGAGAGAUCGAGCCCA | |
| | AGCGGCUGGUGCGGACCGAGAAUCGGCGAGACCUGGGCCUGGUGGGUGCCGUGUUCCU | |
| | GCGGUACACACCCGCCGGCGAGGUGAUGAGCGUGCUGCUGGUGGACACCGACGCCACC | |
| | CAGCAGCAAUUGGCCCAAGGCCCCGUUGCCGGCACACCCAACGUGUUCAGCAGCGACG | |
| | UGCCCAGCGUGGCCCUGCUGCUGUUCCCCAACGGCACCGUGAUCCACCUGCUGGCCUU | |
| | CGACACCCUGCCCAUCGCCACCAUCGCCCCAGGGUUCCUGGCUGCGAGCGCUCUGGGA | |
| | GUGGUGAUGAUCACCGCCGCCUUAGCCGGCAUCCUGCGGGUUGUGCGGACCUGCGUGC | |
| | CCUUCCUGUGGCGGCGGGAG | |
| gL | AUGGGCUUCGUGUGCCUGUUCGGCCUGGUGGUGAUGGGCGCUUGGGGUGCCUGGGGCG | 8 |
| | GUAGCCAGGCCACCGAGUACGUGCUGCGGAGCGUGAUCGCCAAGGAGGUGGGCGACAU | |
| | CCUGCGGGUGCCCUGCAUGCGGACACCCGCCGACGACGUGAGCUGGCGGUACGAGGCC | |
| | CCUAGCGUGAUCGACUACGCCCGGAUCGACGGCAUCUUCCUGCGGUACCACUGCCCCG | |
| | GCCUGGACACCUUCCUGUGGGACCGGCACGCCCAGAGAGCCUACCUGGUGAACCCCUU | |
| | CCUGUUCGCCGCCGGCUUCCUGGAGGACCUGAGCCACAGCGUGUUCCCCGCCGACACC | |
| | CAGGAGACCACAACCCGGCGGGCUCUGUACAAGGAGAUCCGGGACGCCUUAGGCAGCC | |
| | GGAAGCAGGCCGUGAGCCACGCACCAGUGCGGGCUGGCUGCGUGAACUUCGACUACAG | |
| | CCGGACCAGGCGGUGUGUGGGCAGACGGGACCUUCGGCCAGCCAACACCACCAGCACC | |
| | UGGGAGCCUCCUGUGAGCAGCGACGACGAGGCCAGCAGCCAGAGCAAGCCCUUAGCCA | |
| | CCCAGCCACCUGUGCUGGCCCUGUCAAACGCUCCACCCAGGCGGGUGAGCCCUACUCG | |
| | CGGACGGCGAAGGCAUACCCGGCUGAGGCGGAAC | |
| gL Δsp | AUGGCCUGGGGCGGUAGCCAGGCCACCGAGUACGUGCUGCGGAGCGUGAUCGCCAAGG | 9 |
| | AGGUGGGCGACAUCCUGCGGGUGCCCUGCAUGCGGACACCCGCCGACGACGUGAGCUG | |
| | GCGGUACGAGGCCCCUAGCGUGAUCGACUACGCCCGGAUCGACGGCAUCUUCCUGCGG | |
| | UACCACUGCCCCGGCCUGGACACCUUCCUGUGGGACCGGCACGCCCAGAGAGCCUACC | |
| | UGGUGAACCCCUUCCUGUUCGCCGCCGGCUUCCUGGAGGACCUGAGCCACAGCGUGUU | |
| | CCCCGCCGACACCCAGGAGACCACAACCCGGCGGGCUCUGUACAAGGAGAUCCGGGAC | |
| | GCCUUAGGCAGCCGGAAGCAGGCCGUGAGCCACGCACCAGUGCGGGCUGGCUGCGUGA | |
| | ACUUCGACUACAGCCGGACCAGGCGGUGUGUGGGCAGACGGGACCUUCGGCCAGCCAA | |
| | CACCACCAGCACCUGGGAGCCUCCUGUGAGCAGCGACGACGAGGCCAGCAGCCAGAGC | |
| | AAGCCCUUAGCCACCCAGCCACCUGUGCUGGCCCUGUCAAACGCUCCACCCAGGCGGG | |
| | UGAGCCCUACUCGCGGACGGCGAAGGCAUACCCGGCUGAGGCGGAAC | |
| gHgL | AUGGGCUUCGUGUGCCUGUUCGGCCUGGUGGUGAUGGGCGCUUGGGGUGCCUGGGGCG | 10 |
| link | GUAGCCAGGCCACCGAGUACGUGCUGCGGAGCGUGAUCGCCAAGGAGGUGGGCGACAU | |
| | CCUGCGGGUGCCCUGCAUGCGGACACCCGCCGACGACGUGAGCUGGCGGUACGAGGCC | |
| | CCUAGCGUGAUCGACUACGCCCGGAUCGACGGCAUCUUCCUGCGGUACCACUGCCCCG | |
| | GCCUGGACACCUUCCUGUGGGACCGGCACGCCCAGAGAGCCUACCUGGUGAACCCCUU | |
| | CCUGUUCGCCGCCGGCUUCCUGGAGGACCUGAGCCACAGCGUGUUCCCCGCCGACACC | |
| | CAGGAGACCACAACCCGGCGGGCUCUGUACAAGGAGAUCCGGGACGCCUUAGGCAGCC | |
| | GGAAGCAGGCCGUGAGCCACGCACCAGUGCGGGCUGGCUGCGUGAACUUCGACUACAG | |
| | CCGGACCAGGCGGUGUGUGGGCAGACGGGACCUUCGGCCAGCCAACACCACCAGCACC | |
| | UGGGAGCCUCCUGUGAGCAGCGACGACGAGGCCAGCAGCCAGAGCAAGCCCUUAGCCA | |
| | CCCAGCCACCUGUGCUGGCCCUGUCAAACGCUCCACCCAGGCGGGUGAGCCCUACUCG | |
| | CGGACGGCGAAGGCAUACCCGGAGAAGACGGAGAGGCAGCGGCGGAUCUGGCAGCGGC | |
| | GGAAGCAGCGGUGGAGGAAGCGGCAGCGGAGGCUCAGGCGGCUCAGGGUCUGGCGGCA | |
| | GAAGGAGGCGGCGGCACGACACCUACUGGACCGAGCAGAUCGACCCCUGGUUCCUGCA | |
| | CGGCCUGGGCUUGGCUCGGACCUACUGGCGGGACACCAACACCGGACGGCUGUGGCUG | |
| | CCCAACACCCCAGACGCCAGCGAUCCCCAAAGAGGCCGGCUGGCUCCACCUGGCGAGC | |
| | UGAACCUGACCACCGCCAGCGUGCCCAUGCUGCGGUGGUACGCCGAGCGGUUCUGCUU | |
| | CGUGCUGGUGACCACGGCCGAGUUUCCACGGGACCCAGGCCAGCUGCUGUACAUCCCC | |
| | AAGACCUACCUGCUGGGUAGACCCCGGAACGCCAGCCUUCCCGAGCUGCCUGAGGCUG | |
| | GCCCCACCUCACGACCACCCGCCGAGGUGACCCAGCUGAAGGGCCUGAGCCACAAUCC | |
| | CGGAGCCAGCGCUCUGCUGCGGAGCCGGGCUUGGGUGACCUUCGCCGCAGCUCCCGAU | |
| | AGGGAGGGCCUGACAUUCCCGAGAGGCGACGACGGGGCAACCGAAAGACACCCAGACG | |
| | GCCGCAGAAACGCUCCACCUCCCGGACCACCAGCAGGUACGCCCCGGCAUCCCACCAC | |
| | CAACCUGAGCAUCGCCCACCUGCACAACGCCAGCGUGACUUGGCUGGCGGCCAGAGGU | |
| | CUACUGCGGACGCCCGGCAGAUACGUGUACCUGAGCCCCUCUGCCAGCACCUGGCCAG | |
| | UGGGCGUGUGGACCACCGGUGGCCUGGCCUUCGGUUGCGACGCCGCUCUCGUGCGAGC | |
| | CCGGUACGGCAAGGGCUUCAUGGGCCUGGUUAUCAGCAUGCGGGACAGUCCUCCCGCC | |
| | GAGAUCAUCGUGGUGCCCGCCGACAAGACCCUGGCCCGGGUUGGCAACCCCACCGACG | |
| | AGAACGCACCCGCAGUGCUGCCAGGGCCUCCAGCUGGCCCUCGGUACCGGGUGUUCGU | |
| | GCUGGGAGCGCCAACCCCAGCCGACAACGGCAGCGCGUUGGACGCCUUACGACGGGUG | |
| | GCCGGCUAUCCCGAGGAGAGCACCAACUACGCCCAGUACAUGAGCCGGGCCUACGCCG | |
| | AGUUUCUGGGCGAGGACCCAGGGAGCGGCACCGACGCAAGGCCCAGCCUGUUUUGGCG | |
| | ACUGGCAGGCCUCCUGGCCAGCAGCGGCUUCGCCUUCGUGAACGCCGCACACGCGCAC | |
| | GACGCCAUCCGGCUGAGCGACCUGCUGGGCUUCCUGGCCCACUCUCGGGUGCUCGCAG | |
| | GCCUUGCUGCUCGGGGAGCAGCCGGUUGUGCCGCCGACAGCGUGUUCCUGAACGUGAG | |
| | CGUGCUUGACCCUGCGGCCAGGCUGAGACUGGAGGCCCGGCUGGGACACCUGGUGGCC | |
| | GCCAUCCUGGAGCGGGAGCAGAGCCUGGCAGCCCACGCUCUGGGCUACCAGCUGGCCU | |
| | UUGUUCUGGACAGCCCAGCCGCCUACGGAGCCGUUGCCCCUUCCGCAGCCAGACUGAU | |
| | CGACGCCCUGUACGCCGAAUUCUUAGGUGGACGGGCCCUGACCGCACCAAUGGUGCGG | |
| | CGGGCCCUGUUCUACGCCACCGCUGUCCUGCGGGCACCAUUCCUUGCCGGAGCCCCAU | |
| | CUGCCGAGCAGCGGGAGCGUGCCAGAAGGGGUCUGCUGAUCACCACCGCCCUGUGCAC | |
| | CAGCGACGUUGCUGCCGCCACCCACGCUGAUUUACGAGCUGCCCUCGCACGGACCGAC | |
| | CACCAGAAGAACCUGUUCUGGCUGCCCGACCACUUCUCUCCUUGCGCCGCGAGCCUAC | |
| | GGUUCGACCUGGCCGAAGGCGGCUUCAUCCUGGACGCCCUCGCCAUGGCCACCCGGAG | |
| | CGACAUCCCCGCCGACGUGAUGGCCCAACAGACCCGGGGAGUGGCCAGUGCACUGACC | |
| | CGGUGGGCCCACUACAACGCCCUGAUCCGGGCCUUCGUGCCCGAGGCCACCCACCAGU | |
| | GCAGCGGCCCCAGCCACAACGCCGAGCCCCGGAUCCUGGUGCCCAUCACCCACAACGC | |
| | UAGCUACGUGGUGACCCACACUCCCUUGCCCCGGGGAAUCGGCUACAAGCUGACCGGC | |
| | GUGGACGUUCGGCGGCCCCUGUUCAUCACCUACCUGACCGCCACCUGUGAGGGGCACG | |
| | CCCGAGAGAUCGAGCCCAAGCGGCUGGUGCGGACCGAGAAUCGGCGAGACCUGGGCCU | |
| | GGUGGGUGCCGUGUUCCUGCGGUACACACCCGCCGGCGAGGUGAUGAGCGUGCUGCUG | |
| | GUGGACACCGACGCCACCCAGCAGCAAUUGGCCCAAGGCCCCGUUGCCGGCACACCCA | |
| | ACGUGUUCAGCAGCGACGUGCCCAGCGUGGCCCUGCUGCUGUUCCCCAACGGCACCGU | |
| | GAUCCACCUGCUGGCCUUCGACACCCUGCCCAUCGCCACCAUCGCCCCAGGGUUCCUG | |
| | GCUGCGAGCGCUCUGGGAGUGGUGAUGAUCACCGCCGCCUUAGCCGGCAUCCUGCGGG | |
| | UUGUGCGGACCUGCGUGCCCUUCCUGUGGCGGCGGGAG | |
| ICP0 | AUGGAGCCACGACCCGGCACAUCCAGCAGAGCUGACCCCGGUCCUGAACGGCCUCCUC | 11 |
| | GGCAGACCCCUGGCACUCAACCAGCCGCACCACACGCCUGGGGCAUGCUGAACGACAU | |
| | GCAGUGGCUGGCCAGCAGCGACAGCGAGGAGGAGACCGAGGUGGGCAUCAGCGACGAC | |
| | GACCUGCACCGGGACAGCACCAGCGAAGCCGGCAGCACCGACACCGAGAUGUUCGAGG | |
| | CCGGCCUGAUGGACGCCGCUACUCCGCCAGCAAGGCCACCAGCAGAGCGGCAAGGUAG | |
| | CCCCACGCCUGCUGACGCUCAGGGCUCCUGUGGAGGAGGCCCCGUGGGUGAGGAGGAA | |
| | GCAGAGGCCGGCGGUGGUGGAGACGUGAACACUCCCGUGGCCUACCUGAUCGUUGGCG | |
| | UGACAGCCAGCGGCAGCUUCAGCACCAUCCCCAUCGUGAACGAUCCACGGACUCGGGU | |
| | CGAGGCCGAAGCAGCAGUGAGGGCCGGUACAGCCGUGGACUUCAUCUGGACCGGCAAC | |
| | CCUCGGACCGCGCCAAGGAGCCUGAGCCUGGGCGGUCACACCGUCCGGGCUCUGUCAC | |
| | CUACUCCUCCCUGGCCAGGCACCGACGACGAGGACGACGAUCUGGCCGACGUGGACUA | |
| | CGUGCCACCCGCUCCUCGAAGAGCCCCGCGGAGAGGUGGUGGAGGUGCUGGGGCGACC | |
| | CGAGGUACCAGUCAGCCCGCCGCGACUAGACCCGCACCUCCUGGUGCACCCCGCAGCU | |
| | CCAGUUCUGGCGGCGCUCCCCUGAGAGCAGGCGUUGGAUCUGGCAGUGGCGGAGGACC | |
| | CGCUGUUGCCGCGGUAGUCCCACGGGUUGCCAGCCUGCCUCCUGCCGCAGGUGGAGGC | |
| | AGGGCCCAAGCCAGGAGGGUGGGCGAAGACGCAGCCGCAGCCGAAGGCCGAACCCCUC | |
| | CCGCACGGCAACCCAGGGCUGCCCAGGAGCCACCCAUCGUGAUCAGCGACUCUCCGCC | |
| | UCCCAGUCCACGGAGACCCGCUGGUCCUGGCCCUCUGAGCUUCGUGAGCAGCAGCUCA | |
| | GCCCAGGUGAGCUCUGGGCCGGGUGGAGGAGGACUGCCCCAAUCAAGUGGCAGAGCCG | |
| | CCCGACCAAGAGCAGCAGUCGCUCCUCGGGUGCGGUCUCCACCUAGGGCAGCAGCUGC | |
| | UCCCGUGGUGAGCGCCUCAGCUGACGCAGCUGGUCCAGCUCCACCCGCCGUUCCCGUC | |
| | GACGCUCACCGUGCGCCGAGAAGCCGGAUGACCCAGGCCCAGACCGACACCCAAGCCC | |
| | AGAGCCUCGGCCGUGCAGGAGCAACCGACGCAAGAGGCAGCGGCGGUCCAGGUGCAGA | |
| | GGGUGGAUCCGGACCUGCGGCCAGCAGCAGUGCCAGCAGUUCUGCGGCACCUCGGAGC | |
| | CCACUGGCCCCACAAGGGGUGGGCGCCAAACGUGCAGCACCGCGUCGUGCCCCAGACA | |
| | GCGAUAGCGGAGACCGGGGACACGGACCUCUCGCACCAGCUAGUGCCGGCGCAGCACC | |
| | UCCUAGCGCUAGCCCCAGCAGCCAAGCCGCCGUAGCCGCUGCUAGCUCAAGCAGCGCA | |
| | UCAAGCAGUAGCGCGAGCUCAAGCUCCGCCUCUAGCUCGAGCGCAAGCUCCAGCAGCG | |
| | CCAGCAGUAGCAGCGCCUCGAGCAGCUCUGCCAGUUCCUCCGCCGGAGGAGCUGGAGG | |
| | CAGCGUGGCCUCUGCUAGUGGCGCCGGUGAGCGGAGAGAGACCAGCUUAGGACCGCGU | |
| | GCGGCAGCACCUCGGGGACCUCGGAAGUGUGCGCGCAAGACUCGGCACGCCGAAGGAG | |
| | GCCCUGAACCCGGAGCCAGAGAUCCUGCACCAGGCCUGACCCGGUACCUGCCCAUCGC | |
| | CGGCGUGAGCAGCGUGGUGGCCCUGGCACCCUACGUGAACAAGACCGUGACCGGAGAC | |
| | UGCCUGCCCGUGCUGGACAUGGAGACCGGCCACAUCGGCGCCUACGUGGUGCUGGUGG | |
| | ACCAGACCGGCAACGUGGCCGACCUGCUGAGAGCAGCCGCUCCAGCCUGGAGUCGGCG | |
| | GACCCUGCUGCCAGAGCACGCCAGAAACUGCGUGCGGCCGCCUGACUACCCCACACCG | |
| | CCCGCAAGCGAGUGGAACAGCCUGUGGAUGACACCCGUGGGCAACAUGCUGUUCGACC | |
| | AGGGCACUCUGGUGGGCGCACUGGACUUUCACGGCCUGCGGUCUCGGCACCCCUGGUC | |
| | UCGGGAACAAGGCGCUCCUGCGCCUGCUGGUGACGCACCUGCUGGCCACGGCGAG | |
| ICP0 | AUGAACACUCCCGUGGCCUACCUGAUCGUGGGUGUGACCGCCAGCGGCAGCUUCAGCA | 12 |
| Variant | CCAUCCCCAUCGUGAACGAUCCCCGGACGCGCGUCGAAGCAGAAGCCGCCGUGAGAGC | |
| | GGGUACCGCCGUGGACUUCAUCUGGACCGGCAAUCCUCGGACCGCUCCUCGGAGCCUG | |
| | AGCCUGGGCGGACACACCGUGAGGGCCCUUAGCCCCACCCCUCCUUGGCCCGGAACCG | |
| | ACGACGAGGACGACGACCUGGCCGACGUGGACUACGUGCCUCCGGCUCCUAGACGCGC | |
| | UCCUAGAAGGGGAGGUGGCGGUGCUGGAGCAACUAGGGGAACCAGCCAGCCUGCCGCU | |
| | ACAAGACCCGCACCUCCUGGUGCACCGCGGAGCAGCAGUAGCGGUGGCGCACCUCUGC | |
| | GGGCAGGCGUUGGAUCAGGCUCCGGAGGCGGACCAGCUGUGGCCGCCGUGGUUCCUCG | |
| | GGUGGCCAGCCUACCACCUGCAGCCGGUGGAGGCAGAGCGCAAGCUCGACGGGUGGGA | |
| | GAGGACGCAGCUGCUGCCGAAGGCCGGACACCACCCGCUAGGCAACCACGGGCAGCCC | |
| | AGGAGCCUCCCAUCGUGAUCAGCGACAGCCCGCCACCAAGUCCCCGGAGACCUGCCGG | |
| | ACCUGGACCCCUGAGCUUCGUGAGCAGCAGCAGCGCCCAGGUGUCAAGCGGUCCAGGA | |
| | GGCGGAGGCUUACCCCAGAGCAGCGGGAGGGCUGCAAGACCACGCGCUGCAGUGGCUC | |
| | CGCGGGUGAGAUCUCCGCCACGUGCCGCUGCAGCCCCUGUGGUGAGCGCAUCCGCUGA | |
| | CGCUGCCGGUCCUGCACCUCCUGCCGUGCCCGUUGACGCCCAUAGAGCUCCCCGGAGC | |
| | CGGAUGACCCAGGCCCAGACCGACACCCAAGCUCAAAGCCUGGGUCGUGCCGGUGCAA | |
| | CAGACGCCAGGGGCAGUGGUGGACCAGGUGCAGAGGGCGGGCCUGGAGUUCCACGGGG | |
| | AACCAACACCCCUGGUGCCGCUCCACACGCUGCCGAAGGGGCUGCUGCUGGCGGAGGC | |
| | AGUGGAGGUGGCUCUCCAGCCCCUGGACUGACCCGGUACCUGCCCAUCGCCGGCGUGA | |
| | GCAGCGUGGUGGCCCUGGCUCCCUACGUGAACAAGACCGUGACCGGCGACUGCUUACC | |
| | CGUGCUGGACAUGGAGACCGGCCACAUCGGCGCCUACGUGGUGCUGGUGGACCAGACC | |
| | GGCAACGUGGCCGACCUCCUGAGAGCCGCCGCACCAGCUUGGAGCCGGAGAACCCUGC | |
| | UGCCUGAGCACGCCCGGAACUGCGUGCGGCCUCCCGACUACCCCACACCGCCUGCCAG | |
| | CGAGUGGAACAGCCUGUGGAUGACGCCCGUGGGCAACAUGCUGUUCGACCAGGGCACC | |
| | CUGGUGGGAGCCCUGGACUUCCACGGCCUGAGGAGCCGGCACCCUUGGAGCCGGGAGC | |
| | AAGGCGCACCC | |
| ICP4 | AUGAGCGCCGAGCAACAAGGACGGGGCGCAGAAGUGGCCAUGGCCGACGAGGACGGAG | 13 |
| | GAAGACUCCGGGCCGCAGCAGAAACCACCGGAGGUCCUGGCUCACCCGAUCCAGCAGA | |
| | CGGCCCUCCUCCAACCCCAAAUCCGGAUCGAAGACCAGCUGCUCGGCCAGGCUUUGGG | |
| | UGGCACGGAGGACCUGAGGAGAACGAGGACGAAGCGGACGCCUCAGGCGAGGCUGUGG | |
| | ACGAGCCCGCUGCUGACGGUGUGGUGAGCCCACGGCAACUGGCCCUGCUGGCCAGCAU | |
| | GGUGGACGAGGCCGUGCGGACCAUCCCCAGUCCACCUCCAGAGCGAGACGGGGCCCAA | |
| | GAGGAGGCCGCCAGAAGCCCCAGUCCUCCACGCACCCCAAGCAUGCGGGCUGACUACG | |
| | GCGAGGAGAACGCCGGAAGGUGGGUUCGGGGCCCAGAGACCACCUCUGCCGUGCGGGG | |
| | UGCUUACCCCGACCCCAUGGCCAGCCUAAGCCCUAGACCACCUGCACCAGCUGCUCGG | |
| | GCUCCUGCCUCUGCAGCAGAUCACGCUGCCGGCGGAACUCUGGGAGCCGACGACGAGG | |
| | AAGCCGGUGUGCCUGCUCGCGCACCUGGAGCUGCUCCUCGACCCUCUCCUCCGAGGGC | |
| | UGAACCAGCGCCAGCUCGGACACCAGCCGCAACCGCAGGACGGCUGGAAAGGCGUCGU | |
| | GCCAGAGCAGCCGUGGCUGGCAGAGACGCCACAGGCCGCUUUACCGCCGGACGGCCAA | |
| | GACGGGUGGAGUUGGACGCAGACGCCGCUAGCGGUGCUUUCUACGCCCGGUACCGAGA | |
| | CGGCUACGUUAGCGGAGAACCUUGGCCUGGAGCAGGACCACCGCCACCUGGCAGAGUG | |
| | CUGUACGGUGGACUGGGCGACAGCCGUCCUGGCCUGUGGGGAGCUCCCGAAGCAGAGG | |
| | AAGCCCGGGCAAGAUUCGAAGCCAGCGGAGCUCCAGCUCCUGUGUGGGCACCCGAGUU | |
| | AGGAGACGCCGCCCAGCAGUACGCCCUGAUCACCCGGCUGCUGUACACCCCAGACGCC | |
| | GAAGCCAUGGGCUGGCUGCAGAAUCCAAGAGUGGCUCCCGGAGACGUGGCCCUGGACC | |
| | AGGCCUGCUUCCGGAUUUCCGGCGCCGCACGGAAUAGCAGCAGCUUCAUCUCUGGCAG | |
| | CGUUGCCCGAGCCGUGCCACACCUGGGCUACGCCAUGGCUGCGGGCAGGUUUGGCUGG | |
| | GGCCUGGCUCACGUGGCCGCUGCCGUAGCAAUGAGCCGGCGGUACGACAGAGCCCAGA | |
| | AGGGCUUCCUGCUGACCAGCCUGCGGAGAGCCUACGCUCCCCUUCUGGCUCGGGAGAA | |
| | CGCCGCUUUAACGGGCGCACGGACUCCUGACGACGGAGGCGACGCGAACCGCCACGAC | |
| | GGUGACGACGCCAGAGGGAAGCCUCCCCUCCCUAGCGCUUCCCCAGCCGACGAACGAG | |
| | CUGUGCCAGCGGGUUACGGCGCUGCCGGAGUACUGGCUGCGCUUGGAAGACUGAGCGC | |
| | CGCACCUGCAUCUGCGCCUGCUGGCGCUAGAGCUGAGGCCGGUAGAGUGGCCGUGGAG | |
| | UGCCUGGCCGCCUGCAGAGGCAUCCUGGAAGCCCUGGCCGAGGGCUUUGACGGCGAUU | |
| | UAGCCGCCGUGCCCGGAUUGGCAGGAGCACGCCCAGCUGCACCUCCACGACCUGGUCC | |
| | UGCGGGUGCUGCUGCUCCUCCGCACGCAGACGCUCCCAGACUACGCGCCUGGCUUCGG | |
| | GAGCUGCGGUUCGUUCGGGACGCCCUGGUGCUAAUGCGCCUGAGAGGCGAUCUGCGCG | |
| | UAGCGGGAGGAAGCGAAGCCGCUGUGGCUGCUGUUCGGGCCGUGAGCCUGGUGGCUGG | |
| | AGCUCUAGGCCCCGCAUUACCCCGGAGCCCUAGACUGCUGAGCAGCGACCUGCUGUUC | |
| | CAGAACCAGAGCUUGCGGCCCUUACUGGCUGACACCGUGGCAGCGGCUGACUCCCUUG | |
| | CUGCCCCAGCUAGCGCACCCAGAGAGGCUGCUGACGCUCCAAGGCCCGCUGCAGCACG | |
| | ACCCGCUGCACUCACACGGAGGCCUGCCGAAGGCCCUGACCCACAGGGCGGUUGGAGG | |
| | CGACAGCCUCCUGGCCCCUCACAUACGCCCGCACCUAGCGCUGCCGCUCUGGAGGCCU | |
| | ACUGCGCUCCUAGAGCCGUGGCCGAGCUGACCGACCAUCCCCUGUUUCCGGCACCUUG | |
| | GAGACCCGCCCUCAUGUUCGACCCUAGAGCCCUGGCUAGCCUUGCAGCGCGGUGUGCA | |
| | GCUCCUCCUCCAGGAGGGGCUCCAGCUGCCUUUGGGCCACUCCGCGCUAGUGGACCCC | |
| | UCAGAAGGGCCGCUGCCUGGAUGCGUCAGGUGCCCGAUCCCGAGGACGUGCGGGUGGU | |
| | GAUCCUGUAUAGUCCCCUGCCUGGCGAGGAUUUGGCUGCCGGAAGAGCAGGAGGCGGA | |
| | CCACCUCCUGAGUGGAGUGCCGAACGGGGAGGCCUGAGCUGCCUGCUGGCUGCCCUGG | |
| | GCAACCGGCUGUGCGGACCUGCCACUGCCGCUUGGGCCGGAAACUGGACUGGCGCUCC | |
| | CGACGUGUCGGCACUCGGAGCGCAAGGCGUGCUGCUGCUGUCCACCAGAGACCUCGCA | |
| | UUCGCAGGGGCCGUGGAGUUCCUUGGCCUCUUGGCGGGUGCUUGCGACCGGCGGCUGA | |
| | UCGUGGUGAACGCCGUUCGCGCAGCUGCUUGGCCUGCAGCAGCCCCAGUGGUGAGCCG | |
| | GCAGCACGCCUACCUGGCCUGUGAGGUGCUGCCAGCCGUGCAGUGCGCCGUAAGGUGG | |
| | CCCGCUGCUCGAGACCUGCGGCGGACUGUGCUGGCCAGCGGUAGGGUGUUUGGACCCG | |
| | GCGUGUUCGCACGAGUGGAGGCAGCACACGCUCGGCUGUACCCCGACGCUCCUCCACU | |
| | GCGGCUUUGCAGGGGCGCUAACGUGCGGUACAGGGUGCGGACCAGGUUCGGCCCCGAC | |
| | ACACUGGUGCCCAUGAGUCCCCGGGAAUACCGGCGCGCUGUGUUGCCCGCCCUUGACG | |
| | GAAGAGCUGCCGCUUCCGGAGCUGGAGACGCCAUGGCCCCUGGUGCACCAGACUUCUG | |
| | CGAGGACGAGGCCCACAGCCAUCGGGCUUGUGCACGGUGGGGUCUGGGAGCCCCACUG | |
| | AGACCCGUGUACGUAGCCCUGGGCAGAGACGCUGUACGCGGAGGCCCAGCAGAGCUGA | |
| | GGGGACCCAGACGGGAGUUUUGUGCCCGGGCUCUGCUGGAGCCAGACGGCGACGCUCC | |
| | ACCUCUGGUCCUGAGAGACGACGCAGACGCUGGGCCUCCACCUCAGAUCCGGUGGGCU | |
| | UCUGCAGCUGGACGGGCAGGAACCGUGCUGGCUGCAGCCGGUGGUGGCGUGGAGGUUG | |
| | UCGGGACUGCCGCUGGUCUGGCUACACCUCCACGACGCGAGCCCGUGGACAUGGACGC | |
| | CGAGCUGGAGGACGACGACGACGGCCUGUUCGGCGAG | |
| ICP4 | AUGCGGGUGCUGUACGGUGGCCUGGGCGACUCAAGACCUGGCCUCUGGGGCGCACCUG | 14 |
| Variant | AGGCAGAGGAAGCGCGGGCUCGUUUUGAGGCAUCUGGUGCCCCUGCCCCUGUGUGGGC | |
| | CCCAGAGCUGGGAGACGCCGCCCAGCAGUACGCCCUGAUCACCCGGCUGCUGUACACA | |
| | CCCGACGCCGAGGCCAUGGGCUGGCUGCAGAACCCUCGGGUGGCUCCAGGGGACGUGG | |
| | CCCUGGACCAGGCCUGCUUCAGAAUAAGCGGGGCCGCUCGGAACAGCAGCAGCUUCAU | |
| | CAGCGGCAGCGUUGCUCGAGCCGUGCCUCACCUGGGCUACGCAAUGGCCGCCGGCAGA | |
| | UUCGGCUGGGGCCUGGCUCACGUCGCUGCGGCAGUGGCUAUGAGCCGGCGGUACGACA | |
| | GGGCCCAGAAGGGCUUCCUGUUGACAAGCCUGCGCCGCGCAUACGCUCCACUGUUAGC | |
| | CCGCGAGAACGCCGCACUGACGGGAGCCCGGACUCCUGACGACGGCGGAGACGCCAAC | |
| | AGACACGACGGGGACGACGCAAGAGGUAAACCGCCCCUGCCCAGUGCUGCGGCCUCUC | |
| | CCGCUGACGAGAGAGCCGUGCCUGCAGGCUACGGCGCAGCUGGGGUGCUGGCAGCACU | |
| | GGGCAGGCUGUCAGCCGCCCCUGCUAGCGCUCCAGCUGGCGCUAGGGCUGAAGCAGGC | |
| | AGGGUGGCCGUGGAGUGCUUAGCCGCCUGUCGGGGCAUCCUGGAGGCCCUGGCCGAAG | |
| | GCUUUGACGGGGACCUGGCUGCGGUGCCUGGCUUGGCCGGUGCCAGACCUGCAGCACC | |
| | UCCCCGACCUGGUCCAGCAGGCGCAGCAGCUCCUCCUCACGCUGACGCCCCACGCCUG | |
| | AGAGCCUGGCUGCGGGAGCUGCGGUUCGUGCGGGACGCCCUGGUGCUGAUGCGGCUGA | |
| | GAGGCGACCUGCGUGUUGCUGGCGGUUCCGAGGCAGCUGUGGCCGCAGUGAGAGCCGU | |
| | UAGCCUGGUGGCCGGCGCAUUAGGACCAGCCCUGCCACGUAGCCCGAGACUGCUGAGC | |
| | AGCGACCUGCUGUUCCAGAAUCAGUCUUUGGGUGGAGGGUCCGGUGGAGGACCUCCUC | |
| | CAGGUGGAGCACCUGCCGCCUUCGGCCCUUUAAGGGCGUCUGGCCCUCUGCGUAGAGC | |
| | AGCCGCCUGGAUGCGGCAGGUGCCCGAUCCCGAGGACGUGCGGGUGGUGAUCCUGUAC | |
| | AGUCCCCUGCCCGGCGAAGAUCUCGCAGCCGGAAGAGCCGGAGGCGGACCACCACCUG | |
| | AGUGGAGCGCCGAACGAGGUGGGCUGAGCUGUCUUCUCGCCGCGCUGGGCAACAGACU | |
| | CUGUGGACCCGCUACAGCCGCCUGGGCUGGCAAUUGGACAGGUGCCCCGGACGUAUCC | |
| | GCCCUGGGCGCACAAGGCGUGCUGCUGCUGAGCACCCGGGAUCUGGCAUUCGCCGGCG | |
| | CCGUGGAGUUUCUGGGACUGCUGGCAGGUGCCUGUGACCGGCGGCUGAUCGUGGUCAA | |
| | CGCAGUCAGAGCCGCUGACUGGCCUGCGGACGGACCUGUGGUGAGCCGGCAGCACGCC | |
| | UACCUGGCCUGCGAGGUGCUGCCAGCCGUGCAGUGUGCAGUUCGGUGGCCUGCCGCCA | |
| | GAGACCUGCGGCGGACCGUGUUAGCCAGCGGCCGGGUUUUCGGUCCCGGAGUGUUCGC | |
| | UCGGGUGGAAGCAGCCCACGCACGGCUGUACCCCGACGCCCCACCUCUGCGGCUGUGC | |
| | AGAGGAGCAAACGUGCGGUACCGGGUGCGGACACGGUUCGGCCCAGACACCCUGGUGC | |
| | CCAUGUCACCCCGGGAGUACCGGAGAGCCGUGUUACCUGCCCUUGACGGAAGGGCAGC | |
| | CGCUUCUGGGGCCGGUGACGCUAUGGCUCCUGGCGCUCCCGACUUCUGCGAGGACGAA | |
| | GCCCACAGCCACCGAGCCUGCGCCAGGUGGGGUCUCGGUGCCCCACUGAGACCCGUGU | |
| | ACGUGGCUCUGGGACGGGACGCUGUACGGGGUGGGCCCGCUGAACUUAGAGGGCCACG | |
| | GCGGGAGUUCUGUGCACGGGCCCUGCUGGAG | |
| gB 2P#1 | AUGCGGGGUGGCGGACUGGUGUGCGCCCUGGUGGUCGGUGCACUUGUGGCCGCUGUGG | 15 |
| | CUAGCGCUGCACCAGCCGCACCCAGAGCCAGCGGAGGUGUGGCCGCGACCGUUGCAGC | |
| | CAACGGAGGCCCGGCUUCUCAGCCUCCUCCUGUGCCCAGCCCCGCUACCACCAAGGCC | |
| | CGUAAGCGGAAGACCAAGAAGCCUCCCAAGCGGCCCGAGGCUACACCACCACCCGACG | |
| | CCAACGCAACUGUUGCUGCUGGCCACGCCACCCUGAGGGCCCACCUGCGGGAGAUCAA | |
| | GGUGGAGAACGCCGACGCCCAGUUCUACGUGUGUCCACCUCCAACCGGCGCCACCGUG | |
| | GUACAGUUCGAGCAACCUCGCCGGUGCCCCACUCGACCCGAGGGCCAGAACUACACCG | |
| | AGGGCAUCGCCGUGGUGUUCAAGGAGAACAUCGCCCCAUACAAGUUCAAGGCCACCAU | |
| | GUACUACAAGGACGUGACCGUGAGCCAGGUGUGGUUCGGCCACCGGUACAGCCAGUUC | |
| | AUGGGCAUCUUCGAGGAUCGGGCCCCAGUGCCCUUCGAGGAGGUGAUCGACAAGAUCA | |
| | ACGCCAAGGGCGUGUGCCGGAGCACCGCCAAGUACGUGCGGAACAACAUGGAGACCAC | |
| | CGCCUUCCACCGGGACGACCACGAGACCGACAUGGAGCUGAAGCCCGCCAAGGUGGCC | |
| | ACACGGACAAGCCGGGGCUGGCACACCACCGACCUGAAGUACAACCCAAGCCGCGUGG | |
| | AGGCGUUCCACCGGUACGGCACCACCGUGAACUGCAUCGUGGAGGAGGUGGACGCCCG | |
| | GAGCGUGUACCCCUACGACGAAUUCGUGCUGGCCACCGGCGACUUCGUGUACAUGAGC | |
| | CCCUUCUACGGCUACCGGGAGGGCAGCCACACCGAGCACACCAGCUACGCCGCCGACC | |
| | GGUUCAAGCAGGUGGACGGCUUCUACGCCCGGGACCUGACCACUAAAGCCCGGGCCAC | |
| | CUCACCAACCACCCGGAACCUGCUGACCACCCCAAAGUUCACCGUGGCCUGGGACUGG | |
| | GUGCCCAAGCGACCCGCCGUGUGCACCAUGACCAAGUGGCAGGAAGUGGACGAGAUGC | |
| | UGCGGGCCGAAUACGGCGGCAGCUUCCGGUUCAGCAGCGACGCCAUCAGCACCACCUU | |
| | CACCACCAACCUGACCCAGUACAGCCUGAGCCGGGUGGAUCUGGGCGACUGCAUCGGC | |
| | AGAGACGCUCGCGAGGCCAUCGACCGGAUGUUCGCCCGGAAGUACAACGCCACCCACA | |
| | UCAAGGUGGGCCAGCCCCAGUACUACCUCGCCACCGGCGGCUUCCUGAUCGCCUACCA | |
| | GCCCUUGCUGAGCAACACCCUGGCCGAGCUGUACGUGCGGGAGUACAUGCGGGAGCAG | |
| | GACCGGAAGCCCCGGAACGCCACACCCGCCCCUCUGAGAGAAGCCCCUUCCGCCAACG | |
| | CCAGCGUGGAGCGGAUCAAGACCACCAGCAGCAUCGAGUUCGCCCGGCUGCAGUUCAC | |
| | CUACAACCACAUCCAGCGGCACGUGAACGACAUGCUGGGUCGGCCUCCCGUGGCUUGG | |
| | UGCGAGCUGCAGAACCACGAGCUGACCCUGUGGAACGAGGCCCGGAAGCUGAACCCCA | |
| | ACGCCAUCGCCAGCGCUACUGUGGGCCGGAGGGUAAGCGCUCGGAUGCUGGGCGACGU | |
| | GAUGGCCGUGAGCACGUGUGUGCCCGUGGCCCCAGACAACGUGAUCGUGCAGAACAGC | |
| | AUGCGGGUGAGUAGCCGGCCUGGAACCUGCUACAGCCGGCCCCUGGUGUCUUUCCGGU | |
| | ACGAGGACCAGGGUCCCCUGAUCGAGGGCCAGCUGGGCGAGAACAACGAGCUGCGGCU | |
| | GACUCGCGACGCUCUGGAGCCCUGCACCGUGGGCCACAGACGGUACUUCAUCUUCGGC | |
| | GGCGGCUACGUGUACUUCGAGGAGUACGCCUACAGCCACCAGCUGAGCCGGGCCGACG | |
| | UGACCACCGUGAGCACCUUCAUCGACCUGAACAUCACCAUGCUGGAGGACCACGAGUU | |
| | CGUGCCCCUGGAGGUGUACACCCGGCACGAGAUCAAGGACAGCGGCCUGCUGGAUUAC | |
| | ACCGAGGUGCAGCGGCGGAACCAGCUGCACGACCUGCGGUUCGCCGACAUCGACACCG | |
| | UGAUACGGGCCGACGCAAACGCCGCCAUGUUCGCCGGCCUGUGCGCCUUCUUCGAGGG | |
| | CAUGGGCGACCUGGGCAGAGCCGUGGGCAAGGUGGUGAUGGGCGUGGUGGGCGGAGUG | |
| | GUAAGCGCCGUGAGCGGCGUGAGCAGCUUCAUGAGCAACCCCUUCGGUGCCCUGGCAG | |
| | UGGGCCUGCUGGUGCUUGCUGGCCUGGUCGCUGCCUUCUUCGCCUUCCGGUACGUGCU | |
| | ACAGCUGCAGCGGAACCCCAUGAAGGCCCUGUACCCUCUGACCACCAAGGAGCUGAAG | |
| | ACCUCAGACCCUGGCGGCGUUGGAGGAGAGGGUGAGGAGGGAGCAGAGGGCGGAGGCU | |
| | UCGACGAGGCCAAGCUGGCUGAGGCCCGGGAGAUGAUCCGGUACAUGGCCCUGGUGAG | |
| | CGCCAUGGAGCGGACCGAGCACAAGGCCAGAAAGAAGGGCACCAGCGCCCUGCUGAGC | |
| | AGCAAGGUGACCAACAUGGUGCUGCGGAAGCGGAACAAGGCCCGGUACUCACCCCUGC | |
| | ACAACGAGGACGAGGCCGGCGACGAAGACGAGCUG | |
| gB 2P#2 | AUGCGGGGUGGCGGACUGGUGUGCGCCCUGGUGGUCGGUGCACUUGUGGCCGCUGUGG | 16 |
| | CUAGCGCUGCACCAGCCGCACCCAGAGCCAGCGGAGGUGUGGCCGCGACCGUUGCAGC | |
| | CAACGGAGGCCCGGCUUCUCAGCCUCCUCCUGUGCCCAGCCCCGCUACCACCAAGGCC | |
| | CGUAAGCGGAAGACCAAGAAGCCUCCCAAGCGGCCCGAGGCUACACCACCACCCGACG | |
| | CCAACGCAACUGUUGCUGCUGGCCACGCCACCCUGAGGGCCCACCUGCGGGAGAUCAA | |
| | GGUGGAGAACGCCGACGCCCAGUUCUACGUGUGUCCACCUCCAACCGGCGCCACCGUG | |
| | GUACAGUUCGAGCAACCUCGCCGGUGCCCCACUCGACCCGAGGGCCAGAACUACACCG | |
| | AGGGCAUCGCCGUGGUGUUCAAGGAGAACAUCGCCCCAUACAAGUUCAAGGCCACCAU | |
| | GUACUACAAGGACGUGACCGUGAGCCAGGUGUGGUUCGGCCACCGGUACAGCCAGUUC | |
| | AUGGGCAUCUUCGAGGAUCGGGCCCCAGUGCCCUUCGAGGAGGUGAUCGACAAGAUCA | |
| | ACGCCAAGGGCGUGUGCCGGAGCACCGCCAAGUACGUGCGGAACAACAUGGAGACCAC | |
| | CGCCUUCCACCGGGACGACCACGAGACCGACAUGGAGCUGAAGCCCGCCAAGGUGGCC | |
| | ACACGGACAAGCCGGGGCUGGCACACCACCGACCUGAAGUACAACCCAAGCCGCGUGG | |
| | AGGCGUUCCACCGGUACGGCACCACCGUGAACUGCAUCGUGGAGGAGGUGGACGCCCG | |
| | GAGCGUGUACCCCUACGACGAAUUCGUGCUGGCCACCGGCGACUUCGUGUACAUGAGC | |
| | CCCUUCUACGGCUACCGGGAGGGCAGCCACACCGAGCACACCAGCUACGCCGCCGACC | |
| | GGUUCAAGCAGGUGGACGGCUUCUACGCCCGGGACCUGACCACUAAAGCCCGGGCCAC | |
| | CUCACCAACCACCCGGAACCUGCUGACCACCCCAAAGUUCACCGUGGCCUGGGACUGG | |
| | GUGCCCAAGCGACCCGCCGUGUGCACCAUGACCAAGUGGCAGGAAGUGGACGAGAUGC | |
| | UGCGGGCCGAAUACGGCGGCAGCUUCCGGUUCAGCAGCGACGCCAUCAGCACCACCUU | |
| | CACCACCAACCUGACCCAGUACAGCCUGAGCCGGGUGGAUCUGGGCGACUGCAUCGGC | |
| | AGAGACGCUCGCGAGGCCAUCGACCGGAUGUUCGCCCGGAAGUACAACGCCACCCACA | |
| | UCAAGGUGGGCCAGCCCCAGUACUACCUCGCCACCGGCGGCUUCCUGAUCGCCUACCA | |
| | GCCCUUGCUGAGCAACACCCUGGCCGAGCUGUACGUGCGGGAGUACAUGCGGGAGCAG | |
| | GACCGGAAGCCCCGGAACGCCACACCCGCCCCUCUGAGAGAAGCCCCUUCCGCCAACG | |
| | CCAGCGUGGAGCGGAUCAAGACCACCAGCAGCAUCGAGUUCGCCCGGCUGCAGUUCAC | |
| | CUACAACCACAUCCAGCGGCACGUGAACGACCCUCCCGGUCGGAUCGCCGUGGCUUGG | |
| | UGCGAGCUGCAGAACCACGAGCUGACCCUGUGGAACGAGGCCCGGAAGCUGAACCCCA | |
| | ACGCCAUCGCCAGCGCUACUGUGGGCCGGAGGGUAAGCGCUCGGAUGCUGGGCGACGU | |
| | GAUGGCCGUGAGCACGUGUGUGCCCGUGGCCCCAGACAACGUGAUCGUGCAGAACAGC | |
| | AUGCGGGUGAGUAGCCGGCCUGGAACCUGCUACAGCCGGCCCCUGGUGUCUUUCCGGU | |
| | ACGAGGACCAGGGUCCCCUGAUCGAGGGCCAGCUGGGCGAGAACAACGAGCUGCGGCU | |
| | GACUCGCGACGCUCUGGAGCCCUGCACCGUGGGCCACAGACGGUACUUCAUCUUCGGC | |
| | GGCGGCUACGUGUACUUCGAGGAGUACGCCUACAGCCACCAGCUGAGCCGGGCCGACG | |
| | UGACCACCGUGAGCACCUUCAUCGACCUGAACAUCACCAUGCUGGAGGACCACGAGUU | |
| | CGUGCCCCUGGAGGUGUACACCCGGCACGAGAUCAAGGACAGCGGCCUGCUGGAUUAC | |
| | ACCGAGGUGCAGCGGCGGAACCAGCUGCACGACCUGCGGUUCGCCGACAUCGACACCG | |
| | UGAUACGGGCCGACGCAAACGCCGCCAUGUUCGCCGGCCUGUGCGCCUUCUUCGAGGG | |
| | CAUGGGCGACCUGGGCAGAGCCGUGGGCAAGGUGGUGAUGGGCGUGGUGGGCGGAGUG | |
| | GUAAGCGCCGUGAGCGGCGUGAGCAGCUUCAUGAGCAACCCCUUCGGUGCCCUGGCAG | |
| | UGGGCCUGCUGGUGCUUGCUGGCCUGGUCGCUGCCUUCUUCGCCUUCCGGUACGUGCU | |
| | ACAGCUGCAGCGGAACCCCAUGAAGGCCCUGUACCCUCUGACCACCAAGGAGCUGAAG | |
| | ACCUCAGACCCUGGCGGCGUUGGAGGAGAGGGUGAGGAGGGAGCAGAGGGCGGAGGCU | |
| | UCGACGAGGCCAAGCUGGCUGAGGCCCGGGAGAUGAUCCGGUACAUGGCCCUGGUGAG | |
| | CGCCAUGGAGCGGACCGAGCACAAGGCCAGAAAGAAGGGCACCAGCGCCCUGCUGAGC | |
| | AGCAAGGUGACCAACAUGGUGCUGCGGAAGCGGAACAAGGCCCGGUACUCACCCCUGC | |
| | ACAACGAGGACGAGGCCGGCGACGAAGACGAGCUG | |
| gB 2P#3 | AUGCGGGGUGGCGGACUGGUGUGCGCCCUGGUGGUCGGUGCACUUGUGGCCGCUGUGG | 17 |
| | CUAGCGCUGCACCAGCCGCACCCAGAGCCAGCGGAGGUGUGGCCGCGACCGUUGCAGC | |
| | CAACGGAGGCCCGGCUUCUCAGCCUCCUCCUGUGCCCAGCCCCGCUACCACCAAGGCC | |
| | CGUAAGCGGAAGACCAAGAAGCCUCCCAAGCGGCCCGAGGCUACACCACCACCCGACG | |
| | CCAACGCAACUGUUGCUGCUGGCCACGCCACCCUGAGGGCCCACCUGCGGGAGAUCAA | |
| | GGUGGAGAACGCCGACGCCCAGUUCUACGUGUGUCCACCUCCAACCGGCGCCACCGUG | |
| | GUACAGUUCGAGCAACCUCGCCGGUGCCCCACUCGACCCGAGGGCCAGAACUACACCG | |
| | AGGGCAUCGCCGUGGUGUUCAAGGAGAACAUCGCCCCAUACAAGUUCAAGGCCACCAU | |
| | GUACUACAAGGACGUGACCGUGAGCCAGGUGUGGUUCGGCCACCGGUACAGCCAGUUC | |
| | AUGGGCAUCUUCGAGGAUCGGGCCCCAGUGCCCUUCGAGGAGGUGAUCGACAAGAUCA | |
| | ACGCCAAGGGCGUGUGCCGGAGCACCGCCAAGUACGUGCGGAACAACAUGGAGACCAC | |
| | CGCCUUCCACCGGGACGACCACGAGACCGACAUGGAGCUGAAGCCCGCCAAGGUGGCC | |
| | ACACGGACAAGCCGGGGCUGGCACACCACCGACCUGAAGUACAACCCAAGCCGCGUGG | |
| | AGGCGUUCCACCGGUACGGCACCACCGUGAACUGCAUCGUGGAGGAGGUGGACGCCCG | |
| | GAGCGUGUACCCCUACGACGAAUUCGUGCUGGCCACCGGCGACUUCGUGUACAUGAGC | |
| | CCCUUCUACGGCUACCGGGAGGGCAGCCACACCGAGCACACCAGCUACGCCGCCGACC | |
| | GGUUCAAGCAGGUGGACGGCUUCUACGCCCGGGACCUGACCACUAAAGCCCGGGCCAC | |
| | CUCACCAACCACCCGGAACCUGCUGACCACCCCAAAGUUCACCGUGGCCUGGGACUGG | |
| | GUGCCCAAGCGACCCGCCGUGUGCACCAUGACCAAGUGGCAGGAAGUGGACGAGAUGC | |
| | UGCGGGCCGAAUACGGCGGCAGCUUCCGGUUCAGCAGCGACGCCAUCAGCACCACCUU | |
| | CACCACCAACCUGACCCAGUACAGCCUGAGCCGGGUGGAUCUGGGCGACUGCAUCGGC | |
| | AGAGACGCUCGCGAGGCCAUCGACCGGAUGUUCGCCCGGAAGUACAACGCCACCCACA | |
| | UCAAGGUGGGCCAGCCCCAGUACUACCUCGCCACCGGCGGCUUCCUGAUCGCCUACCA | |
| | GCCCUUGCUGAGCAACACCCUGGCCGAGCUGUACGUGCGGGAGUACAUGCGGGAGCAG | |
| | GACCGGAAGCCCCGGAACGCCACACCCGCCCCUCUGAGAGAAGCCCCUUCCGCCAACG | |
| | CCAGCGUGGAGCGGAUCAAGACCACCAGCAGCAUCGAGUUCGCCCGGCUGCAGUUCAC | |
| | CUACAACCACAUCCAGCGGCCUCCCAACGACAUGCUGGGUCGGAUCGCCGUGGCUUGG | |
| | UGCGAGCUGCAGAACCACGAGCUGACCCUGUGGAACGAGGCCCGGAAGCUGAACCCCA | |
| | ACGCCAUCGCCAGCGCUACUGUGGGCCGGAGGGUAAGCGCUCGGAUGCUGGGCGACGU | |
| | GAUGGCCGUGAGCACGUGUGUGCCCGUGGCCCCAGACAACGUGAUCGUGCAGAACAGC | |
| | AUGCGGGUGAGUAGCCGGCCUGGAACCUGCUACAGCCGGCCCCUGGUGUCUUUCCGGU | |
| | ACGAGGACCAGGGUCCCCUGAUCGAGGGCCAGCUGGGCGAGAACAACGAGCUGCGGCU | |
| | GACUCGCGACGCUCUGGAGCCCUGCACCGUGGGCCACAGACGGUACUUCAUCUUCGGC | |
| | GGCGGCUACGUGUACUUCGAGGAGUACGCCUACAGCCACCAGCUGAGCCGGGCCGACG | |
| | UGACCACCGUGAGCACCUUCAUCGACCUGAACAUCACCAUGCUGGAGGACCACGAGUU | |
| | CGUGCCCCUGGAGGUGUACACCCGGCACGAGAUCAAGGACAGCGGCCUGCUGGAUUAC | |
| | ACCGAGGUGCAGCGGCGGAACCAGCUGCACGACCUGCGGUUCGCCGACAUCGACACCG | |
| | UGAUACGGGCCGACGCAAACGCCGCCAUGUUCGCCGGCCUGUGCGCCUUCUUCGAGGG | |
| | CAUGGGCGACCUGGGCAGAGCCGUGGGCAAGGUGGUGAUGGGCGUGGUGGGCGGAGUG | |
| | GUAAGCGCCGUGAGCGGCGUGAGCAGCUUCAUGAGCAACCCCUUCGGUGCCCUGGCAG | |
| | UGGGCCUGCUGGUGCUUGCUGGCCUGGUCGCUGCCUUCUUCGCCUUCCGGUACGUGCU | |
| | ACAGCUGCAGCGGAACCCCAUGAAGGCCCUGUACCCUCUGACCACCAAGGAGCUGAAG | |
| | ACCUCAGACCCUGGCGGCGUUGGAGGAGAGGGUGAGGAGGGAGCAGAGGGCGGAGGCU | |
| | UCGACGAGGCCAAGCUGGCUGAGGCCCGGGAGAUGAUCCGGUACAUGGCCCUGGUGAG | |
| | CGCCAUGGAGCGGACCGAGCACAAGGCCAGAAAGAAGGGCACCAGCGCCCUGCUGAGC | |
| | AGCAAGGUGACCAACAUGGUGCUGCGGAAGCGGAACAAGGCCCGGUACUCACCCCUGC | |
| | ACAACGAGGACGAGGCCGGCGACGAAGACGAGCUG | |
| gB 2P#4 | AUGCGGGGUGGCGGACUGGUGUGCGCCCUGGUGGUCGGUGCACUUGUGGCCGCUGUGG | 18 |
| | CUAGCGCUGCACCAGCCGCACCCAGAGCCAGCGGAGGUGUGGCCGCGACCGUUGCAGC | |
| | CAACGGAGGCCCGGCUUCUCAGCCUCCUCCUGUGCCCAGCCCCGCUACCACCAAGGCC | |
| | CGUAAGCGGAAGACCAAGAAGCCUCCCAAGCGGCCCGAGGCUACACCACCACCCGACG | |
| | CCAACGCAACUGUUGCUGCUGGCCACGCCACCCUGAGGGCCCACCUGCGGGAGAUCAA | |
| | GGUGGAGAACGCCGACGCCCAGUUCUACGUGUGUCCACCUCCAACCGGCGCCACCGUG | |
| | GUACAGUUCGAGCAACCUCGCCGGUGCCCCACUCGACCCGAGGGCCAGAACUACACCG | |
| | AGGGCAUCGCCGUGGUGUUCAAGGAGAACAUCGCCCCAUACAAGUUCAAGGCCACCAU | |
| | GUACUACAAGGACGUGACCGUGAGCCAGGUGUGGUUCGGCCACCGGUACAGCCAGUUC | |
| | AUGGGCAUCUUCGAGGAUCGGGCCCCAGUGCCCUUCGAGGAGGUGAUCGACAAGAUCA | |
| | ACGCCAAGGGCGUGUGCCGGAGCACCGCCAAGUACGUGCGGAACAACAUGGAGACCAC | |
| | CGCCUUCCACCGGGACGACCACGAGACCGACAUGGAGCUGAAGCCCGCCAAGGUGGCC | |
| | ACACGGACAAGCCGGGGCUGGCACACCACCGACCUGAAGUACAACCCAAGCCGCGUGG | |
| | AGGCGUUCCACCGGUACGGCACCACCGUGAACUGCAUCGUGGAGGAGGUGGACGCCCG | |
| | GAGCGUGUACCCCUACGACGAAUUCGUGCUGGCCACCGGCGACUUCGUGUACAUGAGC | |
| | CCCUUCUACGGCUACCGGGAGGGCAGCCACACCGAGCACACCAGCUACGCCGCCGACC | |
| | GGUUCAAGCAGGUGGACGGCUUCUACGCCCGGGACCUGACCACUAAAGCCCGGGCCAC | |
| | CUCACCAACCACCCGGAACCUGCUGACCACCCCAAAGUUCACCGUGGCCUGGGACUGG | |
| | GUGCCCAAGCGACCCGCCGUGUGCACCAUGACCAAGUGGCAGGAAGUGGACGAGAUGC | |
| | UGCGGGCCGAAUACGGCGGCAGCUUCCGGUUCAGCAGCGACGCCAUCAGCACCACCUU | |
| | CACCACCAACCUGACCCAGUACAGCCUGAGCCGGGUGGAUCUGGGCGACUGCAUCGGC | |
| | AGAGACGCUCGCGAGGCCAUCGACCGGAUGUUCGCCCGGAAGUACAACGCCACCCACA | |
| | UCAAGGUGGGCCAGCCCCAGUACUACCUCGCCACCGGCGGCUUCCUGAUCGCCUACCA | |
| | GCCCUUGCUGAGCAACACCCUGGCCGAGCUGUACGUGCGGGAGUACAUGCGGGAGCAG | |
| | GACCGGAAGCCCCGGAACGCCACACCCGCCCCUCUGAGAGAAGCCCCUUCCGCCAACG | |
| | CCAGCGUGGAGCGGAUCAAGACCACCAGCAGCAUCGAGUUCGCCCGGCUGCAGUUCAC | |
| | CUACAACCCUCCCCAGCGGCACGUGAACGACAUGCUGGGUCGGAUCGCCGUGGCUUGG | |
| | UGCGAGCUGCAGAACCACGAGCUGACCCUGUGGAACGAGGCCCGGAAGCUGAACCCCA | |
| | ACGCCAUCGCCAGCGCUACUGUGGGCCGGAGGGUAAGCGCUCGGAUGCUGGGCGACGU | |
| | GAUGGCCGUGAGCACGUGUGUGCCCGUGGCCCCAGACAACGUGAUCGUGCAGAACAGC | |
| | AUGCGGGUGAGUAGCCGGCCUGGAACCUGCUACAGCCGGCCCCUGGUGUCUUUCCGGU | |
| | ACGAGGACCAGGGUCCCCUGAUCGAGGGCCAGCUGGGCGAGAACAACGAGCUGCGGCU | |
| | GACUCGCGACGCUCUGGAGCCCUGCACCGUGGGCCACAGACGGUACUUCAUCUUCGGC | |
| | GGCGGCUACGUGUACUUCGAGGAGUACGCCUACAGCCACCAGCUGAGCCGGGCCGACG | |
| | UGACCACCGUGAGCACCUUCAUCGACCUGAACAUCACCAUGCUGGAGGACCACGAGUU | |
| | CGUGCCCCUGGAGGUGUACACCCGGCACGAGAUCAAGGACAGCGGCCUGCUGGAUUAC | |
| | ACCGAGGUGCAGCGGCGGAACCAGCUGCACGACCUGCGGUUCGCCGACAUCGACACCG | |
| | UGAUACGGGCCGACGCAAACGCCGCCAUGUUCGCCGGCCUGUGCGCCUUCUUCGAGGG | |
| | CAUGGGCGACCUGGGCAGAGCCGUGGGCAAGGUGGUGAUGGGCGUGGUGGGCGGAGUG | |
| | GUAAGCGCCGUGAGCGGCGUGAGCAGCUUCAUGAGCAACCCCUUCGGUGCCCUGGCAG | |
| | UGGGCCUGCUGGUGCUUGCUGGCCUGGUCGCUGCCUUCUUCGCCUUCCGGUACGUGCU | |
| | ACAGCUGCAGCGGAACCCCAUGAAGGCCCUGUACCCUCUGACCACCAAGGAGCUGAAG | |
| | ACCUCAGACCCUGGCGGCGUUGGAGGAGAGGGUGAGGAGGGAGCAGAGGGCGGAGGCU | |
| | UCGACGAGGCCAAGCUGGCUGAGGCCCGGGAGAUGAUCCGGUACAUGGCCCUGGUGAG | |
| | CGCCAUGGAGCGGACCGAGCACAAGGCCAGAAAGAAGGGCACCAGCGCCCUGCUGAGC | |
| | AGCAAGGUGACCAACAUGGUGCUGCGGAAGCGGAACAAGGCCCGGUACUCACCCCUGC | |
| | ACAACGAGGACGAGGCCGGCGACGAAGACGAGCUG | |
| gB Δct | AUGCGGGGUGGCGGACUGGUGUGCGCCCUGGUGGUCGGUGCACUUGUGGCCGCUGUGG | 19 |
| | CUAGCGCUGCACCAGCCGCACCCAGAGCCAGCGGAGGUGUGGCCGCGACCGUUGCAGC | |
| | CAACGGAGGCCCGGCUUCUCAGCCUCCUCCUGUGCCCAGCCCCGCUACCACCAAGGCC | |
| | CGUAAGCGGAAGACCAAGAAGCCUCCCAAGCGGCCCGAGGCUACACCACCACCCGACG | |
| | CCAACGCAACUGUUGCUGCUGGCCACGCCACCCUGAGGGCCCACCUGCGGGAGAUCAA | |
| | GGUGGAGAACGCCGACGCCCAGUUCUACGUGUGUCCACCUCCAACCGGCGCCACCGUG | |
| | GUACAGUUCGAGCAACCUCGCCGGUGCCCCACUCGACCCGAGGGCCAGAACUACACCG | |
| | AGGGCAUCGCCGUGGUGUUCAAGGAGAACAUCGCCCCAUACAAGUUCAAGGCCACCAU | |
| | GUACUACAAGGACGUGACCGUGAGCCAGGUGUGGUUCGGCCACCGGUACAGCCAGUUC | |
| | AUGGGCAUCUUCGAGGAUCGGGCCCCAGUGCCCUUCGAGGAGGUGAUCGACAAGAUCA | |
| | ACGCCAAGGGCGUGUGCCGGAGCACCGCCAAGUACGUGCGGAACAACAUGGAGACCAC | |
| | CGCCUUCCACCGGGACGACCACGAGACCGACAUGGAGCUGAAGCCCGCCAAGGUGGCC | |
| | ACACGGACAAGCCGGGGCUGGCACACCACCGACCUGAAGUACAACCCAAGCCGCGUGG | |
| | AGGCGUUCCACCGGUACGGCACCACCGUGAACUGCAUCGUGGAGGAGGUGGACGCCCG | |
| | GAGCGUGUACCCCUACGACGAAUUCGUGCUGGCCACCGGCGACUUCGUGUACAUGAGC | |
| | CCCUUCUACGGCUACCGGGAGGGCAGCCACACCGAGCACACCAGCUACGCCGCCGACC | |
| | GGUUCAAGCAGGUGGACGGCUUCUACGCCCGGGACCUGACCACUAAAGCCCGGGCCAC | |
| | CUCACCAACCACCCGGAACCUGCUGACCACCCCAAAGUUCACCGUGGCCUGGGACUGG | |
| | GUGCCCAAGCGACCCGCCGUGUGCACCAUGACCAAGUGGCAGGAAGUGGACGAGAUGC | |
| | UGCGGGCCGAAUACGGCGGCAGCUUCCGGUUCAGCAGCGACGCCAUCAGCACCACCUU | |
| | CACCACCAACCUGACCCAGUACAGCCUGAGCCGGGUGGAUCUGGGCGACUGCAUCGGC | |
| | AGAGACGCUCGCGAGGCCAUCGACCGGAUGUUCGCCCGGAAGUACAACGCCACCCACA | |
| | UCAAGGUGGGCCAGCCCCAGUACUACCUCGCCACCGGCGGCUUCCUGAUCGCCUACCA | |
| | GCCCUUGCUGAGCAACACCCUGGCCGAGCUGUACGUGCGGGAGUACAUGCGGGAGCAG | |
| | GACCGGAAGCCCCGGAACGCCACACCCGCCCCUCUGAGAGAAGCCCCUUCCGCCAACG | |
| | CCAGCGUGGAGCGGAUCAAGACCACCAGCAGCAUCGAGUUCGCCCGGCUGCAGUUCAC | |
| | CUACAACCACAUCCAGCGGCACGUGAACGACAUGCUGGGUCGGAUCGCCGUGGCUUGG | |
| | UGCGAGCUGCAGAACCACGAGCUGACCCUGUGGAACGAGGCCCGGAAGCUGAACCCCA | |
| | ACGCCAUCGCCAGCGCUACUGUGGGCCGGAGGGUAAGCGCUCGGAUGCUGGGCGACGU | |
| | GAUGGCCGUGAGCACGUGUGUGCCCGUGGCCCCAGACAACGUGAUCGUGCAGAACAGC | |
| | AUGCGGGUGAGUAGCCGGCCUGGAACCUGCUACAGCCGGCCCCUGGUGUCUUUCCGGU | |
| | ACGAGGACCAGGGUCCCCUGAUCGAGGGCCAGCUGGGCGAGAACAACGAGCUGCGGCU | |
| | GACUCGCGACGCUCUGGAGCCCUGCACCGUGGGCCACAGACGGUACUUCAUCUUCGGC | |
| | GGCGGCUACGUGUACUUCGAGGAGUACGCCUACAGCCACCAGCUGAGCCGGGCCGACG | |
| | UGACCACCGUGAGCACCUUCAUCGACCUGAACAUCACCAUGCUGGAGGACCACGAGUU | |
| | CGUGCCCCUGGAGGUGUACACCCGGCACGAGAUCAAGGACAGCGGCCUGCUGGAUUAC | |
| | ACCGAGGUGCAGCGGCGGAACCAGCUGCACGACCUGCGGUUCGCCGACAUCGACACCG | |
| | UGAUACGGGCCGACGCAAACGCCGCCAUGUUCGCCGGCCUGUGCGCCUUCUUCGAGGG | |
| | CAUGGGCGACCUGGGCAGAGCCGUGGGCAAGGUGGUGAUGGGCGUGGUGGGCGGAGUG | |
| | GUAAGCGCCGUGAGCGGCGUGAGCAGCUUCAUGAGCAACCCCUUCGGUGCCCUGGCAG | |
| | UGGGCCUGCUGGUGCUUGCUGGCCUGGUCGCUGCCUUCUUCGCCUUCCGGUACGUGCU | |
| | ACAGCUGCAGCGGAAC | |
| gB | AUGCGGGGUGGCGGACUGGUGUGCGCCCUGGUGGUCGGUGCACUUGUGGCCGCUGUGG | 20 |
| 2P#2Δct | CUAGCGCUGCACCAGCCGCACCCAGAGCCAGCGGAGGUGUGGCCGCGACCGUUGCAGC | |
| | CAACGGAGGCCCGGCUUCUCAGCCUCCUCCUGUGCCCAGCCCCGCUACCACCAAGGCC | |
| | CGUAAGCGGAAGACCAAGAAGCCUCCCAAGCGGCCCGAGGCUACACCACCACCCGACG | |
| | CCAACGCAACUGUUGCUGCUGGCCACGCCACCCUGAGGGCCCACCUGCGGGAGAUCAA | |
| | GGUGGAGAACGCCGACGCCCAGUUCUACGUGUGUCCACCUCCAACCGGCGCCACCGUG | |
| | GUACAGUUCGAGCAACCUCGCCGGUGCCCCACUCGACCCGAGGGCCAGAACUACACCG | |
| | AGGGCAUCGCCGUGGUGUUCAAGGAGAACAUCGCCCCAUACAAGUUCAAGGCCACCAU | |
| | GUACUACAAGGACGUGACCGUGAGCCAGGUGUGGUUCGGCCACCGGUACAGCCAGUUC | |
| | AUGGGCAUCUUCGAGGAUCGGGCCCCAGUGCCCUUCGAGGAGGUGAUCGACAAGAUCA | |
| | ACGCCAAGGGCGUGUGCCGGAGCACCGCCAAGUACGUGCGGAACAACAUGGAGACCAC | |
| | CGCCUUCCACCGGGACGACCACGAGACCGACAUGGAGCUGAAGCCCGCCAAGGUGGCC | |
| | ACACGGACAAGCCGGGGCUGGCACACCACCGACCUGAAGUACAACCCAAGCCGCGUGG | |
| | AGGCGUUCCACCGGUACGGCACCACCGUGAACUGCAUCGUGGAGGAGGUGGACGCCCG | |
| | GAGCGUGUACCCCUACGACGAAUUCGUGCUGGCCACCGGCGACUUCGUGUACAUGAGC | |
| | CCCUUCUACGGCUACCGGGAGGGCAGCCACACCGAGCACACCAGCUACGCCGCCGACC | |
| | GGUUCAAGCAGGUGGACGGCUUCUACGCCCGGGACCUGACCACUAAAGCCCGGGCCAC | |
| | CUCACCAACCACCCGGAACCUGCUGACCACCCCAAAGUUCACCGUGGCCUGGGACUGG | |
| | GUGCCCAAGCGACCCGCCGUGUGCACCAUGACCAAGUGGCAGGAAGUGGACGAGAUGC | |
| | UGCGGGCCGAAUACGGCGGCAGCUUCCGGUUCAGCAGCGACGCCAUCAGCACCACCUU | |
| | CACCACCAACCUGACCCAGUACAGCCUGAGCCGGGUGGAUCUGGGCGACUGCAUCGGC | |
| | AGAGACGCUCGCGAGGCCAUCGACCGGAUGUUCGCCCGGAAGUACAACGCCACCCACA | |
| | UCAAGGUGGGCCAGCCCCAGUACUACCUCGCCACCGGCGGCUUCCUGAUCGCCUACCA | |
| | GCCCUUGCUGAGCAACACCCUGGCCGAGCUGUACGUGCGGGAGUACAUGCGGGAGCAG | |
| | GACCGGAAGCCCCGGAACGCCACACCCGCCCCUCUGAGAGAAGCCCCUUCCGCCAACG | |
| | CCAGCGUGGAGCGGAUCAAGACCACCAGCAGCAUCGAGUUCGCCCGGCUGCAGUUCAC | |
| | CUACAACCACAUCCAGCGGCACGUGAACGACCCUCCCGGUCGGAUCGCCGUGGCUUGG | |
| | UGCGAGCUGCAGAACCACGAGCUGACCCUGUGGAACGAGGCCCGGAAGCUGAACCCCA | |
| | ACGCCAUCGCCAGCGCUACUGUGGGCCGGAGGGUAAGCGCUCGGAUGCUGGGCGACGU | |
| | GAUGGCCGUGAGCACGUGUGUGCCCGUGGCCCCAGACAACGUGAUCGUGCAGAACAGC | |
| | AUGCGGGUGAGUAGCCGGCCUGGAACCUGCUACAGCCGGCCCCUGGUGUCUUUCCGGU | |
| | ACGAGGACCAGGGUCCCCUGAUCGAGGGCCAGCUGGGCGAGAACAACGAGCUGCGGCU | |
| | GACUCGCGACGCUCUGGAGCCCUGCACCGUGGGCCACAGACGGUACUUCAUCUUCGGC | |
| | GGCGGCUACGUGUACUUCGAGGAGUACGCCUACAGCCACCAGCUGAGCCGGGCCGACG | |
| | UGACCACCGUGAGCACCUUCAUCGACCUGAACAUCACCAUGCUGGAGGACCACGAGUU | |
| | CGUGCCCCUGGAGGUGUACACCCGGCACGAGAUCAAGGACAGCGGCCUGCUGGAUUAC | |
| | ACCGAGGUGCAGCGGCGGAACCAGCUGCACGACCUGCGGUUCGCCGACAUCGACACCG | |
| | UGAUACGGGCCGACGCAAACGCCGCCAUGUUCGCCGGCCUGUGCGCCUUCUUCGAGGG | |
| | CAUGGGCGACCUGGGCAGAGCCGUGGGCAAGGUGGUGAUGGGCGUGGUGGGCGGAGUG | |
| | GUAAGCGCCGUGAGCGGCGUGAGCAGCUUCAUGAGCAACCCCUUCGGUGCCCUGGCAG | |
| | UGGGCCUGCUGGUGCUUGCUGGCCUGGUCGCUGCCUUCUUCGCCUUCCGGUACGUGCU | |
| | ACAGCUGCAGCGGAAC | |
| gB | AUGCGGGGUGGCGGACUGGUGUGCGCCCUGGUGGUCGGUGCACUUGUGGCCGCUGUGG | 21 |
| 2P#4Δct | CUAGCGCUGCACCAGCCGCACCCAGAGCCAGCGGAGGUGUGGCCGCGACCGUUGCAGC | |
| | CAACGGAGGCCCGGCUUCUCAGCCUCCUCCUGUGCCCAGCCCCGCUACCACCAAGGCC | |
| | CGUAAGCGGAAGACCAAGAAGCCUCCCAAGCGGCCCGAGGCUACACCACCACCCGACG | |
| | CCAACGCAACUGUUGCUGCUGGCCACGCCACCCUGAGGGCCCACCUGCGGGAGAUCAA | |
| | GGUGGAGAACGCCGACGCCCAGUUCUACGUGUGUCCACCUCCAACCGGCGCCACCGUG | |
| | GUACAGUUCGAGCAACCUCGCCGGUGCCCCACUCGACCCGAGGGCCAGAACUACACCG | |
| | AGGGCAUCGCCGUGGUGUUCAAGGAGAACAUCGCCCCAUACAAGUUCAAGGCCACCAU | |
| | GUACUACAAGGACGUGACCGUGAGCCAGGUGUGGUUCGGCCACCGGUACAGCCAGUUC | |
| | AUGGGCAUCUUCGAGGAUCGGGCCCCAGUGCCCUUCGAGGAGGUGAUCGACAAGAUCA | |
| | ACGCCAAGGGCGUGUGCCGGAGCACCGCCAAGUACGUGCGGAACAACAUGGAGACCAC | |
| | CGCCUUCCACCGGGACGACCACGAGACCGACAUGGAGCUGAAGCCCGCCAAGGUGGCC | |
| | ACACGGACAAGCCGGGGCUGGCACACCACCGACCUGAAGUACAACCCAAGCCGCGUGG | |
| | AGGCGUUCCACCGGUACGGCACCACCGUGAACUGCAUCGUGGAGGAGGUGGACGCCCG | |
| | GAGCGUGUACCCCUACGACGAAUUCGUGCUGGCCACCGGCGACUUCGUGUACAUGAGC | |
| | CCCUUCUACGGCUACCGGGAGGGCAGCCACACCGAGCACACCAGCUACGCCGCCGACC | |
| | GGUUCAAGCAGGUGGACGGCUUCUACGCCCGGGACCUGACCACUAAAGCCCGGGCCAC | |
| | CUCACCAACCACCCGGAACCUGCUGACCACCCCAAAGUUCACCGUGGCCUGGGACUGG | |
| | GUGCCCAAGCGACCCGCCGUGUGCACCAUGACCAAGUGGCAGGAAGUGGACGAGAUGC | |
| | UGCGGGCCGAAUACGGCGGCAGCUUCCGGUUCAGCAGCGACGCCAUCAGCACCACCUU | |
| | CACCACCAACCUGACCCAGUACAGCCUGAGCCGGGUGGAUCUGGGCGACUGCAUCGGC | |
| | AGAGACGCUCGCGAGGCCAUCGACCGGAUGUUCGCCCGGAAGUACAACGCCACCCACA | |
| | UCAAGGUGGGCCAGCCCCAGUACUACCUCGCCACCGGCGGCUUCCUGAUCGCCUACCA | |
| | GCCCUUGCUGAGCAACACCCUGGCCGAGCUGUACGUGCGGGAGUACAUGCGGGAGCAG | |
| | GACCGGAAGCCCCGGAACGCCACACCCGCCCCUCUGAGAGAAGCCCCUUCCGCCAACG | |
| | CCAGCGUGGAGCGGAUCAAGACCACCAGCAGCAUCGAGUUCGCCCGGCUGCAGUUCAC | |
| | CUACAACCCUCCCCAGCGGCACGUGAACGACAUGCUGGGUCGGAUCGCCGUGGCUUGG | |
| | UGCGAGCUGCAGAACCACGAGCUGACCCUGUGGAACGAGGCCCGGAAGCUGAACCCCA | |
| | ACGCCAUCGCCAGCGCUACUGUGGGCCGGAGGGUAAGCGCUCGGAUGCUGGGCGACGU | |
| | GAUGGCCGUGAGCACGUGUGUGCCCGUGGCCCCAGACAACGUGAUCGUGCAGAACAGC | |
| | AUGCGGGUGAGUAGCCGGCCUGGAACCUGCUACAGCCGGCCCCUGGUGUCUUUCCGGU | |
| | ACGAGGACCAGGGUCCCCUGAUCGAGGGCCAGCUGGGCGAGAACAACGAGCUGCGGCU | |
| | GACUCGCGACGCUCUGGAGCCCUGCACCGUGGGCCACAGACGGUACUUCAUCUUCGGC | |
| | GGCGGCUACGUGUACUUCGAGGAGUACGCCUACAGCCACCAGCUGAGCCGGGCCGACG | |
| | UGACCACCGUGAGCACCUUCAUCGACCUGAACAUCACCAUGCUGGAGGACCACGAGUU | |
| | CGUGCCCCUGGAGGUGUACACCCGGCACGAGAUCAAGGACAGCGGCCUGCUGGAUUAC | |
| | ACCGAGGUGCAGCGGCGGAACCAGCUGCACGACCUGCGGUUCGCCGACAUCGACACCG | |
| | UGAUACGGGCCGACGCAAACGCCGCCAUGUUCGCCGGCCUGUGCGCCUUCUUCGAGGG | |
| | CAUGGGCGACCUGGGCAGAGCCGUGGGCAAGGUGGUGAUGGGCGUGGUGGGCGGAGUG | |
| | GUAAGCGCCGUGAGCGGCGUGAGCAGCUUCAUGAGCAACCCCUUCGGUGCCCUGGCAG | |
| | UGGGCCUGCUGGUGCUUGCUGGCCUGGUCGCUGCCUUCUUCGCCUUCCGGUACGUGCU | |
| | ACAGCUGCAGCGGAAC | |
| gB | AUGCGGGGUGGCGGACUGGUGUGCGCCCUGGUGGUCGGUGCACUUGUGGCCGCUGUGG | 22 |
| 2P#3and4 | CUAGCGCUGCACCAGCCGCACCCAGAGCCAGCGGAGGUGUGGCCGCGACCGUUGCAGC | |
| Δct | CAACGGAGGCCCGGCUUCUCAGCCUCCUCCUGUGCCCAGCCCCGCUACCACCAAGGCC | |
| | CGUAAGCGGAAGACCAAGAAGCCUCCCAAGCGGCCCGAGGCUACACCACCACCCGACG | |
| | CCAACGCAACUGUUGCUGCUGGCCACGCCACCCUGAGGGCCCACCUGCGGGAGAUCAA | |
| | GGUGGAGAACGCCGACGCCCAGUUCUACGUGUGUCCACCUCCAACCGGCGCCACCGUG | |
| | GUACAGUUCGAGCAACCUCGCCGGUGCCCCACUCGACCCGAGGGCCAGAACUACACCG | |
| | AGGGCAUCGCCGUGGUGUUCAAGGAGAACAUCGCCCCAUACAAGUUCAAGGCCACCAU | |
| | GUACUACAAGGACGUGACCGUGAGCCAGGUGUGGUUCGGCCACCGGUACAGCCAGUUC | |
| | AUGGGCAUCUUCGAGGAUCGGGCCCCAGUGCCCUUCGAGGAGGUGAUCGACAAGAUCA | |
| | ACGCCAAGGGCGUGUGCCGGAGCACCGCCAAGUACGUGCGGAACAACAUGGAGACCAC | |
| | CGCCUUCCACCGGGACGACCACGAGACCGACAUGGAGCUGAAGCCCGCCAAGGUGGCC | |
| | ACACGGACAAGCCGGGGCUGGCACACCACCGACCUGAAGUACAACCCAAGCCGCGUGG | |
| | AGGCGUUCCACCGGUACGGCACCACCGUGAACUGCAUCGUGGAGGAGGUGGACGCCCG | |
| | GAGCGUGUACCCCUACGACGAAUUCGUGCUGGCCACCGGCGACUUCGUGUACAUGAGC | |
| | CCCUUCUACGGCUACCGGGAGGGCAGCCACACCGAGCACACCAGCUACGCCGCCGACC | |
| | GGUUCAAGCAGGUGGACGGCUUCUACGCCCGGGACCUGACCACUAAAGCCCGGGCCAC | |
| | CUCACCAACCACCCGGAACCUGCUGACCACCCCAAAGUUCACCGUGGCCUGGGACUGG | |
| | GUGCCCAAGCGACCCGCCGUGUGCACCAUGACCAAGUGGCAGGAAGUGGACGAGAUGC | |
| | UGCGGGCCGAAUACGGCGGCAGCUUCCGGUUCAGCAGCGACGCCAUCAGCACCACCUU | |
| | CACCACCAACCUGACCCAGUACAGCCUGAGCCGGGUGGAUCUGGGCGACUGCAUCGGC | |
| | AGAGACGCUCGCGAGGCCAUCGACCGGAUGUUCGCCCGGAAGUACAACGCCACCCACA | |
| | UCAAGGUGGGCCAGCCCCAGUACUACCUCGCCACCGGCGGCUUCCUGAUCGCCUACCA | |
| | GCCCUUGCUGAGCAACACCCUGGCCGAGCUGUACGUGCGGGAGUACAUGCGGGAGCAG | |
| | GACCGGAAGCCCCGGAACGCCACACCCGCCCCUCUGAGAGAAGCCCCUUCCGCCAACG | |
| | CCAGCGUGGAGCGGAUCAAGACCACCAGCAGCAUCGAGUUCGCCCGGCUGCAGUUCAC | |
| | CUACAACCCUCCCCAGCGGCCUCCCAACGACAUGCUGGGUCGGAUCGCCGUGGCUUGG | |
| | UGCGAGCUGCAGAACCACGAGCUGACCCUGUGGAACGAGGCCCGGAAGCUGAACCCCA | |
| | ACGCCAUCGCCAGCGCUACUGUGGGCCGGAGGGUAAGCGCUCGGAUGCUGGGCGACGU | |
| | GAUGGCCGUGAGCACGUGUGUGCCCGUGGCCCCAGACAACGUGAUCGUGCAGAACAGC | |
| | AUGCGGGUGAGUAGCCGGCCUGGAACCUGCUACAGCCGGCCCCUGGUGUCUUUCCGGU | |
| | ACGAGGACCAGGGUCCCCUGAUCGAGGGCCAGCUGGGCGAGAACAACGAGCUGCGGCU | |
| | GACUCGCGACGCUCUGGAGCCCUGCACCGUGGGCCACAGACGGUACUUCAUCUUCGGC | |
| | GGCGGCUACGUGUACUUCGAGGAGUACGCCUACAGCCACCAGCUGAGCCGGGCCGACG | |
| | UGACCACCGUGAGCACCUUCAUCGACCUGAACAUCACCAUGCUGGAGGACCACGAGUU | |
| | CGUGCCCCUGGAGGUGUACACCCGGCACGAGAUCAAGGACAGCGGCCUGCUGGAUUAC | |
| | ACCGAGGUGCAGCGGCGGAACCAGCUGCACGACCUGCGGUUCGCCGACAUCGACACCG | |
| | UGAUACGGGCCGACGCAAACGCCGCCAUGUUCGCCGGCCUGUGCGCCUUCUUCGAGGG | |
| | CAUGGGCGACCUGGGCAGAGCCGUGGGCAAGGUGGUGAUGGGCGUGGUGGGCGGAGUG | |
| | GUAAGCGCCGUGAGCGGCGUGAGCAGCUUCAUGAGCAACCCCUUCGGUGCCCUGGCAG | |
| | UGGGCCUGCUGGUGCUUGCUGGCCUGGUCGCUGCCUUCUUCGCCUUCCGGUACGUGCU | |
| | ACAGCUGCAGCGGAAC | |
| gI | AUGCCCGGCCGGUCUCUGCAGGGCCUGGCCAUCCUGGGCCUGUGGGUGUGCGCCACCG | 23 |
| | GCUUAGUGGUGCGCGGCCCUACCGUGAGCCUGGUGAGCGACUCACUGGUGGACGCUGG | |
| | CGCCGUUGGUCCACAGGGCUUCGUGGAGGAGGACCUGCGGGUGUUCGGCGAGCUGCAC | |
| | UUCGUGGGCGCACAGGUGCCCCACACCAACUACUACGACGGCAUCAUCGAGCUGUUCC | |
| | ACUAUCCCCUGGGCAACCACUGCCCUCGGGUGGUGCACGUGGUGACCCUGACCGCCUG | |
| | CCCACGGAGACCCGCUGUGGCCUUCACCCUGUGCCGGAGCACCCACCACGCCCACAGC | |
| | CCAGCCUACCCCACCUUAGAGCUGGGCCUUGCCAGGCAGCCUCUGCUGAGAGUGCGGA | |
| | CCGCCACUCGGGACUACGCCGGCCUGUACGUGCUGAGGGUGUGGGUGGGCAGCGCCAC | |
| | CAACGCCAGCCGGUUCGUGCUGGGAGUGGCCCUGAGCGCCAACGGCACCUUCGUGUAC | |
| | AACGGCAGCGACUACGGCUCUUGUGACCCCGCCCAGCUGCCAUUCAGCGCCCCAAGGC | |
| | UGGGACCCAGCAGCGUGUACACCCCAGGAGCCUCCAGACCAACACCACCACGGACCAC | |
| | CACCCCUCCAAGCAGUCCCCGGGAUCCCACCCCAGCUCCAGGCGAUACAGGGACUCCC | |
| | GCUCCAGCAAGCGGCGAGAUCGCCCCUCCCAACAGCACCCGGAGCGCUAGCGAAAGCA | |
| | GGCACCGGCUGACCGUGGCCCAGGUGAUCCAGAUCGCCAUCCCCGCCAGCAUUAUCGC | |
| | CUUUGUGUUCCUGGGCAGCUGCAUCUGCUUCAUACAUCGGUGCCAGCGGCGGUAUAGA | |
| | CGCCCGCGAGGACAGAUCUACAACCCUGGCGGCGUGUCCUGCGCUGUGAACGAGGCCG | |
| | CCAUGGCCAGACUGGGCGCCGAGCUGAGGAGCCACCCCAACACCCCACCAAAGCCUCG | |
| | GCGGCGAAGCUCAAGCAGCACAACCAUGCCAAGCCUGACCAGCAUCGCCGAGGAAAGC | |
| | GAACCCGGGCCAGUGGUGCUGCUGAGCGUGUCCCCACGACCACGGAGUGGUCCCACUG | |
| | CACCUCAGGAGGUG | |
| sgI | AUGCCCGGCCGGUCUCUGCAGGGCCUGGCCAUCCUGGGCCUGUGGGUGUGCGCCACCG | 24 |
| | GCUUAGUGGUGCGCGGCCCUACCGUGAGCCUGGUGAGCGACUCACUGGUGGACGCUGG | |
| | CGCCGUUGGUCCACAGGGCUUCGUGGAGGAGGACCUGCGGGUGUUCGGCGAGCUGCAC | |
| | UUCGUGGGCGCACAGGUGCCCCACACCAACUACUACGACGGCAUCAUCGAGCUGUUCC | |
| | ACUAUCCCCUGGGCAACCACUGCCCUCGGGUGGUGCACGUGGUGACCCUGACCGCCUG | |
| | CCCACGGAGACCCGCUGUGGCCUUCACCCUGUGCCGGAGCACCCACCACGCCCACAGC | |
| | CCAGCCUACCCCACCUUAGAGCUGGGCCUUGCCAGGCAGCCUCUGCUGAGAGUGCGGA | |
| | CCGCCACUCGGGACUACGCCGGCCUGUACGUGCUGAGGGUGUGGGUGGGCAGCGCCAC | |
| | CAACGCCAGCCGGUUCGUGCUGGGAGUGGCCCUGAGCGCCAACGGCACCUUCGUGUAC | |
| | AACGGCAGCGACUACGGCUCUUGUGACCCCGCCCAGCUGCCAUUCAGCGCCCCAAGGC | |
| | UGGGACCCAGCAGCGUGUACACCCCAGGAGCCUCCAGACCAACACCACCACGGACCAC | |
| | CACCCCUCCAAGCAGUCCCCGGGAUCCCACCCCAGCUCCAGGCGAUACAGGGACUCCC | |
| | GCUCCAGCAAGCGGCGAGAUCGCCCCUCCCAACAGCACCCGGAGCGCUAGCGAAAGCA | |
| | GGCACCGG | |
| gE Δct | AUGGCCAGAGGAGCCGGCCUGGUGUUCUUCGUGGGCGUGUGGGUGGUGUCCUGCCUGG | 25 |
| | CCGCAGCUCCCAGAACCAGCUGGAAGCGGGUGACCAGCGGCGAGGACGUGGUGUUACU | |
| | GCCAGCUCCAGCCGAGCGGACUCGGGCCCACAAGCUGCUUUGGGCAGCCGAGCCACUG | |
| | GACGCCUGCGGGCCAUUACGGCCUAGCUGGGUGGCUCUGUGGCCACCUAGACGGGUGC | |
| | UGGAGACCGUGGUGGACGCAGCCUGCAUGCGGGCACCAGAGCCCCUGGCCAUCGCCUA | |
| | CUCUCCACCCUUCCCAGCCGGCGACGAAGGCCUGUACAGCGAGCUGGCUUGGAGAGAC | |
| | CGGGUGGCCGUGGUGAACGAGAGCCUGGUGAUCUACGGCGCCCUGGAGACCGACAGCG | |
| | GCCUGUACACCCUGAGCGUGGUGGGCCUGAGCGACGAGGCCCGGCAAGUGGCCAGCGU | |
| | GGUGCUGGUGGUUGAGCCCGCCCCUGUGCCAACGCCCACUCCCGACGACUACGACGAG | |
| | GAGGACGACGCAGGCGUGAGCGAGCGGACCCCAGUGAGCGUGCCUCCACCCACUCCUC | |
| | CUCGGAGACCUCCCGUGGCUCCACCUACCCAUCCCCGGGUGAUCCCCGAGGUGAGCCA | |
| | CGUGCGGGGCGUGACCGUGCACAUGGAGACACCCGAGGCCAUCCUGUUCGCCCCUGGC | |
| | GAGACCUUCGGCACCAACGUGAGCAUCCACGCCAUAGCCCACGACGACGGCCCUUACG | |
| | CCAUGGACGUCGUGUGGAUGCGGUUCGACGUGCCCAGCAGCUGCGCCGAGAUGCGGAU | |
| | CUACGAGGCCUGCCUGUACCAUCCCCAGCUGCCCGAGUGCCUGAGCCCCGCUGACGCA | |
| | CCCUGCGCCGUGAGCAGCUGGGCCUACCGGCUGGCUGUGCGGAGCUACGCCGGGUGUA | |
| | GCCGGACAACGCCUCCGCCACGGUGCUUCGCCGAGGCCCGGAUGGAACCUGUUCCCGG | |
| | CCUGGCCUGGCUUGCUAGCACCGUGAACCUGGAGUUCCAGCACGCCAGCCCACAACAC | |
| | GCCGGCCUGUACCUGUGCGUGGUGUACGUGGACGACCACAUCCACGCCUGGGGCCACA | |
| | UGACCAUCAGCACCGCCGCCCAGUACCGGAACGCCGUGGUGGAGCAGCAUCUGCCCCA | |
| | GAGGCAGCCCGAGCCAGUGGAGCCCACGAGACCACACGUGCGGGCACCUCACCCUGCU | |
| | CCCAGCGCACGUGGGCCACUGCGGCUGGGAGCUGUGCUGGGCGCCGCUCUGCUGCUGG | |
| | CAGCCCUGGGACUGAGCGCCUGGGCC | |
| gI Δct | AUGCCCGGCCGGUCUCUGCAGGGCCUGGCCAUCCUGGGCCUGUGGGUGUGCGCCACCG | 26 |
| | GCUUAGUGGUGCGCGGCCCUACCGUGAGCCUGGUGAGCGACUCACUGGUGGACGCUGG | |
| | CGCCGUUGGUCCACAGGGCUUCGUGGAGGAGGACCUGCGGGUGUUCGGCGAGCUGCAC | |
| | UUCGUGGGCGCACAGGUGCCCCACACCAACUACUACGACGGCAUCAUCGAGCUGUUCC | |
| | ACUAUCCCCUGGGCAACCACUGCCCUCGGGUGGUGCACGUGGUGACCCUGACCGCCUG | |
| | CCCACGGAGACCCGCUGUGGCCUUCACCCUGUGCCGGAGCACCCACCACGCCCACAGC | |
| | CCAGCCUACCCCACCUUAGAGCUGGGCCUUGCCAGGCAGCCUCUGCUGAGAGUGCGGA | |
| | CCGCCACUCGGGACUACGCCGGCCUGUACGUGCUGAGGGUGUGGGUGGGCAGCGCCAC | |
| | CAACGCCAGCCGGUUCGUGCUGGGAGUGGCCCUGAGCGCCAACGGCACCUUCGUGUAC | |
| | AACGGCAGCGACUACGGCUCUUGUGACCCCGCCCAGCUGCCAUUCAGCGCCCCAAGGC | |
| | UGGGACCCAGCAGCGUGUACACCCCAGGAGCCUCCAGACCAACACCACCACGGACCAC | |
| | CACCCCUCCAAGCAGUCCCCGGGAUCCCACCCCAGCUCCAGGCGAUACAGGGACUCCC | |
| | GCUCCAGCAAGCGGCGAGAUCGCCCCUCCCAACAGCACCCGGAGCGCUAGCGAAAGCA | |
| | GGCACCGGCUGACCGUGGCCCAGGUGAUCCAGAUCGCCAUCCCCGCCAGCAUUAUCGC | |
| | CUUUGUGUUCCUGGGCAGCUGCAUCUGCUUCAUACAU | |
| gC Δct | AUGGCACUUGGACGCGUCGGUCUGGCUGUCGGCCUGUGGGGCCUGCUGUGGGUGGGCG | 27 |
| | UGGUGGUGGUGCUGGCUAACGCCAGCCCCGGACGUACCAUCACCGUGGGGCCCAGAGG | |
| | CAACGCCAGUAACGCCGCGCCAUCAGCCAGCCCGAGAAACGCUAGUGCUCCCCGGACC | |
| | ACGCCAACUCCACCACAGCCCCGGAAGGCCACCAAGAGCAAGGCCAGCACCGCCAAGC | |
| | CCGCUCCACCUCCCAAGACCGGCCCACCCAAGACCAGCAGCGAGCCCGUGCGGUGCAA | |
| | CCGGCACGAUCCUCUGGCCCGGUACGGCUCACGGGUGCAGAUCCGGUGCCGGUUCCCC | |
| | AACAGCACCAGGACCGAGAGCCGGCUGCAGAUCUGGCGGUACGCCACCGCCACAGACG | |
| | CCGAGAUCGGCACCGCCCCAAGCCUGGAGGAGGUGAUGGUGAACGUGUCUGCUCCACC | |
| | UGGCGGCCAGCUGGUGUACGACAGCGCACCCAACCGGACCGAUCCCCACGUGAUCUGG | |
| | GCAGAAGGCGCUGGUCCUGGCGCUAGCCCUCGACUGUACAGCGUGGUCGGCCCACUGG | |
| | GCAGGCAGCGGCUGAUCAUCGAGGAGCUGACCCUGGAGACCCAGGGCAUGUACUACUG | |
| | GGUGUGGGGCCGGACAGAUCGGCCUAGCGCCUACGGGACCUGGGUGCGGGUUCGCGUG | |
| | UUCCGGCCACCUAGCCUGACCAUCCAUCCCCACGCCGUGCUGGAGGGCCAGCCCUUCA | |
| | AAGCCACCUGUACCGCCGCCACCUACUACCCAGGCAACCGGGCCGAGUUCGUGUGGUU | |
| | CGAGGACGGACGGCGCGUGUUCGACCCCGCCCAGAUCCACACCCAGACCCAGGAGAAC | |
| | CCCGACGGCUUCAGCACCGUGAGCACGGUGACCAGCGCUGCCGUUGGAGGCCAAGGCC | |
| | CUCCCAGAACCUUCACCUGCCAGCUGACCUGGCACCGGGACAGCGUGAGCUUUUCUCG | |
| | CCGGAACGCCAGCGGAACUGCCAGCGUGCUGCCACGGCCCACCAUCACCAUGGAGUUC | |
| | ACCGGCGACCACGCCGUGUGCACAGCCGGCUGCGUACCCGAGGGCGUGACCUUCGCCU | |
| | GGUUCCUGGGCGACGACAGCAGCCCCGCCGAGAAGGUGGCCGUGGCCAGCCAGACCAG | |
| | CUGUGGAAGACCCGGAACCGCCACCAUCCGGAGCACCCUGCCCGUGAGCUACGAGCAG | |
| | ACCGAGUACAUCUGUCGGCUGGCCGGCUACCCUGACGGCAUCCCCGUGUUGGAGCACC | |
| | ACGGCAGCCACCAGCCUCCUCCUCGGGAUCCCACCGAGCGGCAGGUGAUCAGGGCCGU | |
| | GGAGGGUGCAGGCAUCGGCGUGGCCGUGCUGGUGGCCGUAGUGCUAGCCGGCACCGCC | |
| | GUGGUAUACCUGACC | |
| gC F327A | AUGGCACUUGGACGCGUCGGUCUGGCUGUCGGCCUGUGGGGCCUGCUGUGGGUGGGCG | 28 |
| Δct | UGGUGGUGGUGCUGGCUAACGCCAGCCCCGGACGUACCAUCACCGUGGGGCCCAGAGG | |
| | CAACGCCAGUAACGCCGCGCCAUCAGCCAGCCCGAGAAACGCUAGUGCUCCCCGGACC | |
| | ACGCCAACUCCACCACAGCCCCGGAAGGCCACCAAGAGCAAGGCCAGCACCGCCAAGC | |
| | CCGCUCCACCUCCCAAGACCGGCCCACCCAAGACCAGCAGCGAGCCCGUGCGGUGCAA | |
| | CCGGCACGAUCCUCUGGCCCGGUACGGCUCACGGGUGCAGAUCCGGUGCCGGUUCCCC | |
| | AACAGCACCAGGACCGAGAGCCGGCUGCAGAUCUGGCGGUACGCCACCGCCACAGACG | |
| | CCGAGAUCGGCACCGCCCCAAGCCUGGAGGAGGUGAUGGUGAACGUGUCUGCUCCACC | |
| | UGGCGGCCAGCUGGUGUACGACAGCGCACCCAACCGGACCGAUCCCCACGUGAUCUGG | |
| | GCAGAAGGCGCUGGUCCUGGCGCUAGCCCUCGACUGUACAGCGUGGUCGGCCCACUGG | |
| | GCAGGCAGCGGCUGAUCAUCGAGGAGCUGACCCUGGAGACCCAGGGCAUGUACUACUG | |
| | GGUGUGGGGCCGGACAGAUCGGCCUAGCGCCUACGGGACCUGGGUGCGGGUUCGCGUG | |
| | UUCCGGCCACCUAGCCUGACCAUCCAUCCCCACGCCGUGCUGGAGGGCCAGCCCUUCA | |
| | AAGCCACCUGUACCGCCGCCACCUACUACCCAGGCAACCGGGCCGAGUUCGUGUGGUU | |
| | CGAGGACGGACGGCGCGUGUUCGACCCCGCCCAGAUCCACACCCAGACCCAGGAGAAC | |
| | CCCGACGGCUUCAGCACCGUGAGCACGGUGACCAGCGCUGCCGUUGGAGGCCAAGGCC | |
| | CUCCCAGAACCUUCACCUGCCAGCUGACCUGGCACCGGGACAGCGUGAGCGCUUCUCG | |
| | CCGGAACGCCAGCGGAACUGCCAGCGUGCUGCCACGGCCCACCAUCACCAUGGAGUUC | |
| | ACCGGCGACCACGCCGUGUGCACAGCCGGCUGCGUACCCGAGGGCGUGACCUUCGCCU | |
| | GGUUCCUGGGCGACGACAGCAGCCCCGCCGAGAAGGUGGCCGUGGCCAGCCAGACCAG | |
| | CUGUGGAAGACCCGGAACCGCCACCAUCCGGAGCACCCUGCCCGUGAGCUACGAGCAG | |
| | ACCGAGUACAUCUGUCGGCUGGCCGGCUACCCUGACGGCAUCCCCGUGUUGGAGCACC | |
| | ACGGCAGCCACCAGCCUCCUCCUCGGGAUCCCACCGAGCGGCAGGUGAUCAGGGCCGU | |
| | GGAGGGUGCAGGCAUCGGCGUGGCCGUGCUGGUGGCCGUAGUGCUAGCCGGCACCGCC | |
| | GUGGUAUACCUGACC | |
| gD Δct | AUGGGUCGGCUGACCAGCGGAGUUGGCACCGCCGCACUCCUGGUGGUGGCCGUAGGCC | 29 |
| | UGCGGGUGGUGUGCGCCAAGUACGCCCUGGCCGACCCAUCCCUGAAGAUGGCCGACCC | |
| | UAACCGGUUCCGGGGCAAGAAUCUGCCCGUGCUGGAUCAGCUGACCGAUCCUCCUGGC | |
| | GUGAAGCGGGUGUACCACAUCCAGCCCAGCCUGGAGGAUCCCUUCCAGCCACCCUCCA | |
| | UCCCCAUCACCGUUUACUACGCCGUGCUGGAGAGAGCCUGCCGCAGCGUGCUGCUGCA | |
| | CGCUCCAUCCGAGGCGCCCCAGAUCGUGCGGGGCGCAAGCGACGAGGCCCGGAAGCAC | |
| | ACCUACAACCUGACCAUCGCCUGGUACCGGAUGGGCGACAACUGCGCCAUCCCUAUCA | |
| | CCGUGAUGGAGUACACCGAGUGCCCCUACAACAAGAGCCUGGGAGUGUGCCCCAUCCG | |
| | GACCCAGCCUCGGUGGUCCUACUACGACAGCUUCAGCGCCGUGUCCGAGGACAACCUG | |
| | GGCUUCCUGAUGCACGCCCCUGCCUUCGAGACCGCCGGCACCUACCUGCGGCUGGUGA | |
| | AGAUCAACGACUGGACCGAGAUCACCCAGUUCAUCCUGGAGCACCGGGCAAGGGCCAG | |
| | CUGCAAGUACGCGCUGCCUCUGCGGAUCCCUCCCGCUGCUUGCCUGACCAGCAAGGCC | |
| | UACCAGCAGGGCGUGACCGUGGACAGCAUCGGCAUGCUGCCCAGGUUCAUCCCCGAGA | |
| | ACCAGCGCACCGUGGCCCUGUACAGCCUGAAGAUCGCAGGCUGGCACGGACCCAAGCC | |
| | UCCUUACACCUCCACCCUGCUGCCACCCGAGCUGAGCGACACCACCAACGCCACCCAG | |
| | CCCGAGCUGGUGCCCGAGGACCCCGAGGACUCCGCCCUGCUGGAGGAUCCGGCCGGCA | |
| | CCGUAAGCUCCCAGAUCCCACCCAACUGGCACAUCCCCAGCAUCCAGGACGUGGCCCC | |
| | ACAUCACGCGCCUGCCGCUCCAAGCAACCCCGGCCUGAUCAUUGGAGCUCUGGCCGGG | |
| | AGCACUCUGGCGGUGCUGGUGAUCGGCGGCAUCGCCUUCUGGGUG | |
| gC | AUGGCCCUUGGACGGGUAGGCCUAGCCGUGGGCCUGUGGGGCCUACUGUGGGUGGGUG | 30 |
| | UGGUCGUGGUGCUGGCCAAUGCCUCCCCCGGACGCACGAUAACGGUGGGCCCGCGAGG | |
| | CAACGCGAGCAAUGCUGCCCCCUCCGCGUCCCCGCGGAACGCAUCCGCCCCCCGAACC | |
| | ACACCCACGCCCCCACAACCCCGCAAAGCGACGAAAUCCAAGGCCUCCACCGCCAAAC | |
| | CGGCUCCGCCCCCCAAGACCGGACCCCCGAAGACAUCCUCGGAGCCCGUGCGAUGCAA | |
| | CCGCCACGACCCGCUGGCCCGGUACGGCUCGCGGGUGCAAAUCCGAUGCCGGUUUCCC | |
| | AACUCCACGAGGACUGAGUCCCGUCUCCAGAUCUGGCGUUAUGCCACGGCGACGGACG | |
| | CCGAAAUCGGAACAGCGCCUAGCUUAGAAGAGGUGAUGGUGAACGUGUCGGCCCCGCC | |
| | CGGGGGCCAACUGGUGUAUGACAGUGCCCCCAACCGAACGGACCCGCAUGUAAUCUGG | |
| | GCGGAGGGCGCCGGCCCGGGCGCCAGCCCGCGCCUGUACUCGGUUGUCGGCCCGCUGG | |
| | GUCGGCAGCGGCUCAUCAUCGAAGAGUUAACCCUGGAGACACAGGGCAUGUACUAUUG | |
| | GGUGUGGGGCCGGACGGACCGCCCGUCCGCCUACGGGACCUGGGUCCGCGUUCGAGUA | |
| | UUUCGCCCUCCGUCGCUGACCAUCCACCCCCACGCGGUGCUGGAGGGCCAGCCGUUUA | |
| | AGGCGACGUGCACGGCCGCAACCUACUACCCGGGCAACCGCGCGGAGUUCGUCUGGUU | |
| | UGAGGACGGUCGCCGCGUAUUCGAUCCGGCACAGAUACACACGCAGACGCAGGAGAAC | |
| | CCCGACGGCUUUUCCACCGUCUCCACCGUGACCUCCGCGGCCGUCGGCGGGCAGGGCC | |
| | CCCCUCGCACCUUCACCUGCCAGCUGACGUGGCACCGCGACUCCGUGUCGUUCUCUCG | |
| | GCGCAACGCCAGCGGCACGGCCUCGGUUCUGCCGCGGCCGACCAUUACCAUGGAGUUU | |
| | ACAGGCGACCAUGCGGUCUGCACGGCCGGCUGUGUGCCCGAGGGGGUCACGUUUGCUU | |
| | GGUUCCUGGGGGAUGACUCCUCGCCGGCGGAAAAGGUGGCCGUCGCGUCCCAGACAUC | |
| | GUGCGGGCGCCCCGGCACCGCCACGAUCCGCUCCACCCUGCCGGUCUCGUACGAGCAG | |
| | ACCGAGUACAUCUGUAGACUGGCGGGAUACCCGGACGGAAUUCCGGUCCUAGAGCACC | |
| | ACGGAAGCCACCAGCCCCCGCCGCGGGACCCAACCGAGCGGCAGGUGAUCCGGGCGGU | |
| | GGAGGGGGCGGGGAUCGGAGUGGCUGUCCUUGUCGCGGUGGUUCUGGCCGGGACCGCG | |
| | GUAGUGUACCUGACCCAUGCCUCCUCGGUACGCUAUCGUCGGCUGCGG | |
| sgD | AUGGGUCGGCUGACCAGCGGAGUUGGCACCGCCGCACUCCUGGUGGUGGCCGUAGGCC | 31 |
| | UGCGGGUGGUGUGCGCCAAGUACGCCCUGGCCGACCCAUCCCUGAAGAUGGCCGACCC | |
| | UAACCGGUUCCGGGGCAAGAAUCUGCCCGUGCUGGAUCAGCUGACCGAUCCUCCUGGC | |
| | GUGAAGCGGGUGUACCACAUCCAGCCCAGCCUGGAGGAUCCCUUCCAGCCACCCUCCA | |
| | UCCCCAUCACCGUUUACUACGCCGUGCUGGAGAGAGCCUGCCGCAGCGUGCUGCUGCA | |
| | CGCUCCAUCCGAGGCGCCCCAGAUCGUGCGGGGCGCAAGCGACGAGGCCCGGAAGCAC | |
| | ACCUACAACCUGACCAUCGCCUGGUACCGGAUGGGCGACAACUGCGCCAUCCCUAUCA | |
| | CCGUGAUGGAGUACACCGAGUGCCCCUACAACAAGAGCCUGGGAGUGUGCCCCAUCCG | |
| | GACCCAGCCUCGGUGGUCCUACUACGACAGCUUCAGCGCCGUGUCCGAGGACAACCUG | |
| | GGCUUCCUGAUGCACGCCCCUGCCUUCGAGACCGCCGGCACCUACCUGCGGCUGGUGA | |
| | AGAUCAACGACUGGACCGAGAUCACCCAGUUCAUCCUGGAGCACCGGGCAAGGGCCAG | |
| | CUGCAAGUACGCGCUGCCUCUGCGGAUCCCUCCCGCUGCUUGCCUGACCAGCAAGGCC | |
| | UACCAGCAGGGCGUGACCGUGGACAGCAUCGGCAUGCUGCCCAGGUUCAUCCCCGAGA | |
| | ACCAGCGCACCGUGGCCCUGUACAGCCUGAAGAUCGCAGGCUGGCACGGACCCAAGCC | |
| | UCCUUACACCUCCACCCUGCUGCCACCCGAGCUGAGCGACACCACCAACGCCACCCAG | |
| | CCCGAGCUGGUGCCCGAGGACCCCGAGGACUCCGCCCUGCUGGAGGAUCCGGCCGGCA | |
| | CCGUAAGCUCCCAGAUCCCACCCAACUGGCACAUCCCCAGCAUCCAGGACGUGGCCCC | |
| | ACAUCACGCGCCUGCCGCUCCAAGCAACCCC | |
| gD gp120 | AUGGGUCGGCUGACCAGCGGAGUUGGCACCGCCGCACUCCUGGUGGUGGCCGUAGGCC | 32 |
| | UGCGGGUGGUGUGCGCCAAGUACGCCCUGGCCGACCCAUCCCUGAAGAUGGCCGACCC | |
| | UAACCGGUUCCGGGGCAAGAAUCUGCCCGUGCUGGAUCAGGGCGUGCCCGUGUGGAAG | |
| | GAGGCCACCACCACCCUGUUCUGCGCCAGCGACGCCAAGGCCUACGACACCGAGGUGC | |
| | ACAACGUGUGGGCCACCCACGCCUGUGUGCCCACCGACCCCAACCCACAGGAGGUGGU | |
| | GCUGGUGAACGUGACCGAGAACUUCAACAUGUGGAAGAACGACAUGGUGGAGCAGAUG | |
| | CACGAGGACAUCAUCAGCCUGUGGGACCAGAGCCUGAAGCCCUGCGUGAAGCUGACUC | |
| | CCCUGUGCGUGAGCCUGAAGUGCACCGACCUGAAGAACGACACCAACACCAACAGCAG | |
| | CAGCGGCCGGAUGAUCAUGGAGAAGGGCGAGAUCAAGAACUGCAGCUUCAACAUCAGC | |
| | ACCAGCAUCCGGGGCAAGGUGCAGAAGGAGUACGCCUUCUUCUACAAGCUGGACAUCA | |
| | UCCCCAUCGACAACGACACCACCAGCUACAAGCUGACCAGCUGCAACACCAGCGUGAU | |
| | CACCCAGGCCUGCCCCAAGGUGAGCUUCGAGCCCAUCCCCAUCCACUACUGCGCCCCA | |
| | GCCGGCUUUGCCAUCCUGAAGUGCAACAACAAGACCUUCAACGGCACCGGACCCUGCA | |
| | CGAACGUGAGCACCGUGCAGUGCACCCACGGAAUCCGGCCCGUGGUGAGCACCCAGCU | |
| | GCUGCUGAACGGCAGCCUGGCCGAGGAGGAGGUGGUGAUCCGGAGCGUGAACUUCACC | |
| | GACAACGCCAAGACCAUCAUCGUGCAGCUGAACACCAGCGUGGAGAUCAACUGCACCC | |
| | GGCCCAACAACAACACCCGGAAGCGGAUCAGGAUCCAAAGAGGCCCCGGCAGAGCCUU | |
| | CGUGACCAUCGGCAAGAUCGGCAACAUGCGGCAGGCCCACUGCAACAUCAGCCGGGCC | |
| | AAGUGGAACAACACCCUGAAGCAGAUCGCCAGCAAGCUGCGGGAGCAGUUCGGCAACA | |
| | ACAAGACCAUCAUCUUCAAGCAGAGCUCAGGCGGGGAUCCCGAGAUCGUGACCCACAG | |
| | CUUCAACUGCGGCGGCGAGUUCUUCUACUGCAACAGCACCCAGCUGUUCAAUAGCACC | |
| | UGGUUCAACAGCACCUGGAGCACCGAGGGCAGCAACAACACCGAGGGCAGCGACACCA | |
| | UCACCCUGCCCUGCCGGAUCAAGCAGAUCAUCAACAUGUGGCAGAAGGUGGGCAAGGC | |
| | CAUGUACGCCCCUCCCAUCAGCGGCCAGAUCCGGUGCAGCAGCAACAUCACCGGCCUG | |
| | CUGCUGACCCGGGACGGUGGCAACAGCAACAACGAGAGCGAGAUCUUUAGACCCGGAG | |
| | GCGGCGACAUGCGGGACAACUGGCGGAGCGAGCUGUACAAGUACAAGGUGGUGAAGAU | |
| | CGAGCCACUGGGAGUGGCCCCUACCAAGGCCAAGCGGCGCGUAGUGCAGGGCGGCGGA | |
| | AGUGGUGGCGGACUGAUCAUUGGAGCUCUGGCCGGGAGCACUCUGGCGGUGCUGGUGA | |
| | UCGGCGGCAUCGCCUUCUGGGUGCGUCGGAGAGCCCAGAUGGCCCCUAAGCGGCUGCG | |
| | GCUGCCACACAUACGGGACGACGACGCGCCACCAUCCCACCAGCCCCUGUUCUAC | |
| sgD | AUGGGUCGGCUGACCAGCGGAGUUGGCACCGCCGCACUCCUGGUGGUGGCCGUAGGCC | 33 |
| gp120 | UGCGGGUGGUGUGCGCCAAGUACGCCCUGGCCGACCCAUCCCUGAAGAUGGCCGACCC | |
| | UAACCGGUUCCGGGGCAAGAAUCUGCCCGUGCUGGAUCAGGGCGUGCCCGUGUGGAAG | |
| | GAGGCCACCACCACCCUGUUCUGCGCCAGCGACGCCAAGGCCUACGACACCGAGGUGC | |
| | ACAACGUGUGGGCCACCCACGCCUGUGUGCCCACCGACCCCAACCCACAGGAGGUGGU | |
| | GCUGGUGAACGUGACCGAGAACUUCAACAUGUGGAAGAACGACAUGGUGGAGCAGAUG | |
| | CACGAGGACAUCAUCAGCCUGUGGGACCAGAGCCUGAAGCCCUGCGUGAAGCUGACUC | |
| | CCCUGUGCGUGAGCCUGAAGUGCACCGACCUGAAGAACGACACCAACACCAACAGCAG | |
| | CAGCGGCCGGAUGAUCAUGGAGAAGGGCGAGAUCAAGAACUGCAGCUUCAACAUCAGC | |
| | ACCAGCAUCCGGGGCAAGGUGCAGAAGGAGUACGCCUUCUUCUACAAGCUGGACAUCA | |
| | UCCCCAUCGACAACGACACCACCAGCUACAAGCUGACCAGCUGCAACACCAGCGUGAU | |
| | CACCCAGGCCUGCCCCAAGGUGAGCUUCGAGCCCAUCCCCAUCCACUACUGCGCCCCA | |
| | GCCGGCUUUGCCAUCCUGAAGUGCAACAACAAGACCUUCAACGGCACCGGACCCUGCA | |
| | CGAACGUGAGCACCGUGCAGUGCACCCACGGAAUCCGGCCCGUGGUGAGCACCCAGCU | |
| | GCUGCUGAACGGCAGCCUGGCCGAGGAGGAGGUGGUGAUCCGGAGCGUGAACUUCACC | |
| | GACAACGCCAAGACCAUCAUCGUGCAGCUGAACACCAGCGUGGAGAUCAACUGCACCC | |
| | GGCCCAACAACAACACCCGGAAGCGGAUCAGGAUCCAAAGAGGCCCCGGCAGAGCCUU | |
| | CGUGACCAUCGGCAAGAUCGGCAACAUGCGGCAGGCCCACUGCAACAUCAGCCGGGCC | |
| | AAGUGGAACAACACCCUGAAGCAGAUCGCCAGCAAGCUGCGGGAGCAGUUCGGCAACA | |
| | ACAAGACCAUCAUCUUCAAGCAGAGCUCAGGCGGGGAUCCCGAGAUCGUGACCCACAG | |
| | CUUCAACUGCGGCGGCGAGUUCUUCUACUGCAACAGCACCCAGCUGUUCAAUAGCACC | |
| | UGGUUCAACAGCACCUGGAGCACCGAGGGCAGCAACAACACCGAGGGCAGCGACACCA | |
| | UCACCCUGCCCUGCCGGAUCAAGCAGAUCAUCAACAUGUGGCAGAAGGUGGGCAAGGC | |
| | CAUGUACGCCCCUCCCAUCAGCGGCCAGAUCCGGUGCAGCAGCAACAUCACCGGCCUG | |
| | CUGCUGACCCGGGACGGUGGCAACAGCAACAACGAGAGCGAGAUCUUUAGACCCGGAG | |
| | GCGGCGACAUGCGGGACAACUGGCGGAGCGAGCUGUACAAGUACAAGGUGGUGAAGAU | |
| | CGAGCCACUGGGAGUGGCCCCUACCAAGGCCAAGCGGCGCGUAGUGCAGCGGGAGAAG | |
| | CGG | |
| sICP0 | AUGGUGUUCACCCCUCAGAUCCUGGGCCUGAUGCUGUUCUGGAUCAGCGCCAGCCGGG | 34 |
| Variant | GCAACACUCCCGUGGCCUACCUGAUCGUGGGUGUGACCGCCAGCGGCAGCUUCAGCAC | |
| | CAUCCCCAUCGUGAACGAUCCCCGGACGCGCGUCGAAGCAGAAGCCGCCGUGAGAGCG | |
| | GGUACCGCCGUGGACUUCAUCUGGACCGGCAAUCCUCGGACCGCUCCUCGGAGCCUGA | |
| | GCCUGGGCGGACACACCGUGAGGGCCCUUAGCCCCACCCCUCCUUGGCCCGGAACCGA | |
| | CGACGAGGACGACGACCUGGCCGACGUGGACUACGUGCCUCCGGCUCCUAGACGCGCU | |
| | CCUAGAAGGGGAGGUGGCGGUGCUGGAGCAACUAGGGGAACCAGCCAGCCUGCCGCUA | |
| | CAAGACCCGCACCUCCUGGUGCACCGCGGAGCAGCAGUAGCGGUGGCGCACCUCUGCG | |
| | GGCAGGCGUUGGAUCAGGCUCCGGAGGCGGACCAGCUGUGGCCGCCGUGGUUCCUCGG | |
| | GUGGCCAGCCUACCACCUGCAGCCGGUGGAGGCAGAGCGCAAGCUCGACGGGUGGGAG | |
| | AGGACGCAGCUGCUGCCGAAGGCCGGACACCACCCGCUAGGCAACCACGGGCAGCCCA | |
| | GGAGCCUCCCAUCGUGAUCAGCGACAGCCCGCCACCAAGUCCCCGGAGACCUGCCGGA | |
| | CCUGGACCCCUGAGCUUCGUGAGCAGCAGCAGCGCCCAGGUGUCAAGCGGUCCAGGAG | |
| | GCGGAGGCUUACCCCAGAGCAGCGGGAGGGCUGCAAGACCACGCGCUGCAGUGGCUCC | |
| | GCGGGUGAGAUCUCCGCCACGUGCCGCUGCAGCCCCUGUGGUGAGCGCAUCCGCUGAC | |
| | GCUGCCGGUCCUGCACCUCCUGCCGUGCCCGUUGACGCCCAUAGAGCUCCCCGGAGCC | |
| | GGAUGACCCAGGCCCAGACCGACACCCAAGCUCAAAGCCUGGGUCGUGCCGGUGCAAC | |
| | AGACGCCAGGGGCAGUGGUGGACCAGGUGCAGAGGGCGGGCCUGGAGUUCCACGGGGA | |
| | ACCAACACCCCUGGUGCCGCUCCACACGCUGCCGAAGGGGCUGCUGCUGGCGGAGGCA | |
| | GUGGAGGUGGCUCUCCAGCCCCUGGACUGACCCGGUACCUGCCCAUCGCCGGCGUGAG | |
| | CAGCGUGGUGGCCCUGGCUCCCUACGUGAACAAGACCGUGACCGGCGACUGCUUACCC | |
| | GUGCUGGACAUGGAGACCGGCCACAUCGGCGCCUACGUGGUGCUGGUGGACCAGACCG | |
| | GCAACGUGGCCGACCUCCUGAGAGCCGCCGCACCAGCUUGGAGCCGGAGAACCCUGCU | |
| | GCCUGAGCACGCCCGGAACUGCGUGCGGCCUCCCGACUACCCCACACCGCCUGCCAGC | |
| | GAGUGGAACAGCCUGUGGAUGACGCCCGUGGGCAACAUGCUGUUCGACCAGGGCACCC | |
| | UGGUGGGAGCCCUGGACUUCCACGGCCUGAGGAGCCGGCACCCUUGGAGCCGGGAGCA | |
| | AGGCGCACCC | |
| sICP4 | AUGGUGUUCACCCCUCAGAUCCUGGGCCUGAUGCUGUUCUGGAUCAGCGCCAGCCGGG | 35 |
| Variant | GCCGGGUGCUGUACGGUGGCCUGGGCGACUCAAGACCUGGCCUCUGGGGCGCACCUGA | |
| | GGCAGAGGAAGCGCGGGCUCGUUUUGAGGCAUCUGGUGCCCCUGCCCCUGUGUGGGCC | |
| | CCAGAGCUGGGAGACGCCGCCCAGCAGUACGCCCUGAUCACCCGGCUGCUGUACACAC | |
| | CCGACGCCGAGGCCAUGGGCUGGCUGCAGAACCCUCGGGUGGCUCCAGGGGACGUGGC | |
| | CCUGGACCAGGCCUGCUUCAGAAUAAGCGGGGCCGCUCGGAACAGCAGCAGCUUCAUC | |
| | AGCGGCAGCGUUGCUCGAGCCGUGCCUCACCUGGGCUACGCAAUGGCCGCCGGCAGAU | |
| | UCGGCUGGGGCCUGGCUCACGUCGCUGCGGCAGUGGCUAUGAGCCGGCGGUACGACAG | |
| | GGCCCAGAAGGGCUUCCUGUUGACAAGCCUGCGCCGCGCAUACGCUCCACUGUUAGCC | |
| | CGCGAGAACGCCGCACUGACGGGAGCCCGGACUCCUGACGACGGCGGAGACGCCAACA | |
| | GACACGACGGGGACGACGCAAGAGGUAAACCGCCCCUGCCCAGUGCUGCGGCCUCUCC | |
| | CGCUGACGAGAGAGCCGUGCCUGCAGGCUACGGCGCAGCUGGGGUGCUGGCAGCACUG | |
| | GGCAGGCUGUCAGCCGCCCCUGCUAGCGCUCCAGCUGGCGCUAGGGCUGAAGCAGGCA | |
| | GGGUGGCCGUGGAGUGCUUAGCCGCCUGUCGGGGCAUCCUGGAGGCCCUGGCCGAAGG | |
| | CUUUGACGGGGACCUGGCUGCGGUGCCUGGCUUGGCCGGUGCCAGACCUGCAGCACCU | |
| | CCCCGACCUGGUCCAGCAGGCGCAGCAGCUCCUCCUCACGCUGACGCCCCACGCCUGA | |
| | GAGCCUGGCUGCGGGAGCUGCGGUUCGUGCGGGACGCCCUGGUGCUGAUGCGGCUGAG | |
| | AGGCGACCUGCGUGUUGCUGGCGGUUCCGAGGCAGCUGUGGCCGCAGUGAGAGCCGUU | |
| | AGCCUGGUGGCCGGCGCAUUAGGACCAGCCCUGCCACGUAGCCCGAGACUGCUGAGCA | |
| | GCGACCUGCUGUUCCAGAAUCAGUCUUUGGGUGGAGGGUCCGGUGGAGGACCUCCUCC | |
| | AGGUGGAGCACCUGCCGCCUUCGGCCCUUUAAGGGCGUCUGGCCCUCUGCGUAGAGCA | |
| | GCCGCCUGGAUGCGGCAGGUGCCCGAUCCCGAGGACGUGCGGGUGGUGAUCCUGUACA | |
| | GUCCCCUGCCCGGCGAAGAUCUCGCAGCCGGAAGAGCCGGAGGCGGACCACCACCUGA | |
| | GUGGAGCGCCGAACGAGGUGGGCUGAGCUGUCUUCUCGCCGCGCUGGGCAACAGACUC | |
| | UGUGGACCCGCUACAGCCGCCUGGGCUGGCAAUUGGACAGGUGCCCCGGACGUAUCCG | |
| | CCCUGGGCGCACAAGGCGUGCUGCUGCUGAGCACCCGGGAUCUGGCAUUCGCCGGCGC | |
| | CGUGGAGUUUCUGGGACUGCUGGCAGGUGCCUGUGACCGGCGGCUGAUCGUGGUCAAC | |
| | GCAGUCAGAGCCGCUGACUGGCCUGCGGACGGACCUGUGGUGAGCCGGCAGCACGCCU | |
| | ACCUGGCCUGCGAGGUGCUGCCAGCCGUGCAGUGUGCAGUUCGGUGGCCUGCCGCCAG | |
| | AGACCUGCGGCGGACCGUGUUAGCCAGCGGCCGGGUUUUCGGUCCCGGAGUGUUCGCU | |
| | CGGGUGGAAGCAGCCCACGCACGGCUGUACCCCGACGCCCCACCUCUGCGGCUGUGCA | |
| | GAGGAGCAAACGUGCGGUACCGGGUGCGGACACGGUUCGGCCCAGACACCCUGGUGCC | |
| | CAUGUCACCCCGGGAGUACCGGAGAGCCGUGUUACCUGCCCUUGACGGAAGGGCAGCC | |
| | GCUUCUGGGGCCGGUGACGCUAUGGCUCCUGGCGCUCCCGACUUCUGCGAGGACGAAG | |
| | CCCACAGCCACCGAGCCUGCGCCAGGUGGGGUCUCGGUGCCCCACUGAGACCCGUGUA | |
| | CGUGGCUCUGGGACGGGACGCUGUACGGGGUGGGCCCGCUGAACUUAGAGGGCCACGG | |
| | CGGGAGUUCUGUGCACGGGCCCUGCUGGAG | |

“s” indicates a soluble protein. “Δct” indicates a deletion of the cytoplasmic tail. “Asp” indicates a deletion of the signal peptide.

Some aspects of the present disclosure provide a ribonucleic acid (RNA) comprising an open reading frame comprising a nucleic acid sequence having at least 90%, at least 95%, at least 96%, at least 97%, at least 98%, at least 99%, or 100% identity to the nucleic acid sequence of any one of SEQ ID NOs: 1-35.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 1.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 2.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 3.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 4.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 5.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 6.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 7.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 8.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 9.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 10.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 11.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 12.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 13.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 14.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 15.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 16.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 17.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 18.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 19.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 20.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the amino acid sequence of SEQ ID NO: 21.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 22.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 23.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 24.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 25.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 26.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 27.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 28.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 29.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 30.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 31.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 32.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 33.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 34.

In some embodiments, an RNA (e.g., mRNA) comprises an open reading frame comprising a nucleic acid sequence having at least 90%, at least 95%, at least 96%, at least 97%, at least 98%, at least 99%, or 100% identity to the nucleic acid sequence of SEQ ID NO: 35.

TABLE 2
Amino acid sequences of exemplary HSV protein variants

| | | SEQ |
|---|---|---|
| | | ID |
| Protein | Amino acid sequence | NO. |
| gB WT | MRGGGLVCALVVGALVAAVASAAPAAPRASGGVAATVAANGGPASQPPPVPSPATT | 36 |
| | KARKRKTKKPPKRPEATPPPDANATVAAGHATLRAHLREIKVENADAQFYVCPPPT | |
| | GATVVQFEQPRRCPTRPEGQNYTEGIAVVFKENIAPYKFKATMYYKDVTVSQVWFG | |
| | HRYSQFMGIFEDRAPVPFEEVIDKINAKGVCRSTAKYVRNNMETTAFHRDDHETDM | |
| | ELKPAKVATRTSRGWHTTDLKYNPSRVEAFHRYGTTVNCIVEEVDARSVYPYDEFV | |
| | LATGDFVYMSPFYGYREGSHTEHTSYAADRFKQVDGFYARDLTTKARATSPTTRNL | |
| | LTTPKFTVAWDWVPKRPAVCTMTKWQEVDEMLRAEYGGSFRFSSDAISTTFTTNLT | |
| | QYSLSRVDLGDCIGRDAREAIDRMFARKYNATHIKVGQPQYYLATGGFLIAYQPLL | |
| | SNTLAELYVREYMREQDRKPRNATPAPLREAPSANASVERIKTTSSIEFARLOFTY | |
| | NHIQRHVNDMLGRIAVAWCELQNHELTLWNEARKLNPNAIASATVGRRVSARMLGD | |
| | VMAVSTCVPVAPDNVIVONSMRVSSRPGTCYSRPLVSFRYEDQGPLIEGOLGENNE | |
| | LRLTRDALEPCTVGHRRYFIFGGGYVYFEEYAYSHQLSRADVTTVSTFIDLNITML | |
| | EDHEFVPLEVYTRHEIKDSGLLDYTEVQRRNQLHDLRFADIDTVIRADANAAMFAG | |
| | LCAFFEGMGDLGRAVGKVVMGVVGGVVSAVSGVSSFMSNPFGALAVGLLVLAGLVA | |
| | AFFAFRYVLQLQRNPMKALYPLTTKELKTSDPGGVGGEGEEGAEGGGEDEAKLAEA | |
| | REMIRYMALVSAMERTEHKARKKGTSALLSSKVTNMVLRKRNKARYSPLHNEDEAG | |
| | DEDEL | |
| gB pf | MRGGGLVCALVVGALVAAVASAAPAAPRASGGVAATVAANGGPASQPPPVPSPATT | 37 |
| | KARKRKTKKPPKRPEATPPPDANATVAAGHATLRAHLREIKVENADAQFYVCPPPT | |
| | GATVVQFEQPRRCPTRPEGQNYTEGIAVVFKENIAPYKFKATMYYKDVTVSQVWFG | |
| | HRYSQFMGIFEDRAPVPFEEVIDKINAKGVCRSTAKYVRNNMETTAFHRDDHETDM | |
| | ELKPAKVATRTSRGWHTTDLKYNPSRVEAFHRYGTTVNCIVEEVDARSVYPYDEFV | |
| | LATGDFVYMSPFYGYREGSHTEHTSYAADRFKQVDGFYARDLTTKARATSPTTRNL | |
| | LTTPKFTVAWDWVPKRPAVCTMTKWQEVDEMLRAEYGGSFRFSSDAISTTFTTNLT | |
| | QYSLSRVDLGDCIGRDAREAIDRMFARKYNATHIKVGQPQYYLATGGFLIAYQPLL | |
| | SNTLAELYVREYMREQDRKPRNATPAPLREAPSANASVERIKTTSSIEFARLOFTY | |
| | NHIQRPVNDMLGRIAVAWCELQNHELTLWNEARKLNPNAIASATVGRRVSARMLGD | |
| | VMAVSTCVPVAPDNVIVONSMRVSSRPGTCYSRPLVSFRYEDQGPLIEGOLGENNE | |
| | LRLTRDALEPCTVGHRRYFIFGGGYVYFEEYAYSHQLSRADVTTVSTFIDLNITML | |
| | EDHEFVPLEVYTRHEIKDSGLLDYTEVQRRNQLHDLRFADIDTVIRADANAAMFAG | |
| | LCAFFEGMGDLGRAVGKVVMGVVGGVVSAVSGVSSFMSNPFGALAVGLLVLAGLVA | |
| | AFFAFRYVLQLQRNPMKALYPLTTKELKTSDPGGVGGEGEEGAEGGGFDEAKLAEA | |
| | REMIRYMALVSAMERTEHKARKKGTSALLSSKVTNMVLRKRNKARYSPLHNEDEAG | |
| | DEDEL | |
| gC F327A | MALGRVGLAVGLWGLLWVGVVVVLANASPGRTITVGPRGNASNAAPSASPRNASAP | 38 |
| | RTTPTPPQPRKATKSKASTAKPAPPPKTGPPKTSSEPVRCNRHDPLARYGSRVQIR | |
| | CRFPNSTRTESRLQIWRYATATDAEIGTAPSLEEVMVNVSAPPGGQLVYDSAPNRT | |
| | DPHVIWAEGAGPGASPRLYSVVGPLGRORLIIEELTLETQGMYYWVWGRTDRPSAY | |
| | GTWVRVRVFRPPSLTIHPHAVLEGQPFKATCTAATYYPGNRAEFVWFEDGRRVEDP | |
| | AQIHTQTQENPDGFSTVSTVTSAAVGGQGPPRTFTCOLTWHRDSVSASRRNASGTA | |
| | SVLPRPTITMEFTGDHAVCTAGCVPEGVTFAWFLGDDSSPAEKVAVASQTSCGRPG | |
| | TATIRSTLPVSYEQTEYICRLAGYPDGIPVLEHHGSHQPPPRDPTERQVIRAVEGA | |
| | GIGVAVLVAVVLAGTAVVYLTHASSVRYRRLR | |
| gD WT | MGRLTSGVGTAALLVVAVGLRVVCAKYALADPSLKMADPNRFRGKNLPVLDQLTDP | 39 |
| | PGVKRVYHIQPSLEDPFQPPSIPITVYYAVLERACRSVLLHAPSEAPQIVRGASDE | |
| | ARKHTYNLTIAWYRMGDNCAIPITVMEYTECPYNKSLGVCPIRTOPRWSYYDSFSA | |
| | VSEDNLGFLMHAPAFETAGTYLRLVKINDWTEITQFILEHRARASCKYALPLRIPP | |
| | AACLTSKAYQQGVTVDSIGMLPRFIPENQRTVALYSLKIAGWHGPKPPYTSTLLPP | |
| | ELSDTTNATQPELVPEDPEDSALLEDPAGTVSSQIPPNWHIPSIQDVAPHHAPAAP | |
| | SNPGLIIGALAGSTLAVLVIGGIAFWVRRRAQMAPKRLRLPHIRDDDAPPSHOPLF | |
| | Y | |
| gE | MARGAGLVFFVGVWVVSCLAAAPRTSWKRVTSGEDVVLLPAPAGPEERTRAHKLLW | 40 |
| | AAEPLDACGPLRPSWVALWPPRRVLETVVDAACMRAPEPLAIAYSPPFPAGDEGLY | |
| | SELAWRDRVAVVNESLVIYGALETDSGLYTLSVVGLSDEARQVASVVLVVEPAPVP | |
| | TPTPDDYDEEDDAGVSERTPVSVPPPTPPRRPPVAPPTHPRVIPEVSHVRGVTVHM | |
| | ETPEAILFAPGETFGTNVSIHAIAHDDGPYAMDVVWMRFDVPSSCAEMRIYEACLY | |
| | HPQLPECLSPADAPCAVSSWAYRLAVRSYAGCSRTTPPPRCFAEARMEPVPGLAWL | |
| | ASTVNLEFQHASPQHAGLYLCVVYVDDHIHAWGHMTISTAAQYRNAVVEQHLPQRQ | |
| | PEPVEPTRPHVRAPPPAPSARGPLRLGAVLGAALLLAALGLSAWACMTCWRRRSWR | |
| | AVKSRASATGPTYIRVADSELYADWSSDSEGERDGSLWQDPPERPDSPSTNGSGFE | |
| | ILSPTAPSVYPHSEGRKSRRPLTTFGSGSPGRRHSQASYSSVLW | |
| sgE | MARGAGLVFFVGVWVVSCLAAAPRTSWKRVTSGEDVVLLPAPAGPEERTRAHKLLW | 41 |
| | AAEPLDACGPLRPSWVALWPPRRVLETVVDAACMRAPEPLAIAYSPPFPAGDEGLY | |
| | SELAWRDRVAVVNESLVIYGALETDSGLYTLSVVGLSDEARQVASVVLVVEPAPVP | |
| | TPTPDDYDEEDDAGVSERTPVSVPPPTPPRRPPVAPPTHPRVIPEVSHVRGVTVHM | |
| | ETPEAILFAPGETFGTNVSIHAIAHDDGPYAMDVVWMRFDVPSSCAEMRIYEACLY | |
| | HPQLPECLSPADAPCAVSSWAYRLAVRSYAGCSRTTPPPRCFAEARMEPVPGLAWL | |
| | ASTVNLEFQHASPQHAGLYLCVVYVDDHIHAWGHMTISTAAQYRNAVVEQHLPQRQ | |
| | PEPVEPTRPHVRAPPPAPSARGPLR | |
| gH | MGPGLWVVMGVLVGVAGGHDTYWTEQIDPWFLHGLGLARTYWRDTNTGRLWLPNTP | 42 |
| | DASDPQRGRLAPPGELNLTTASVPMLRWYAERFCFVLVTTAEFPRDPGQLLYIPKT | |
| | YLLGRPRNASLPELPEAGPTSRPPAEVTQLKGLSHNPGASALLRSRAWVTFAAAPD | |
| | REGLTFPRGDDGATERHPDGRRNAPPPGPPAGTPRHPTTNLSIAHLHNASVTWLAA | |
| | RGLLRTPGRYVYLSPSASTWPVGVWTTGGLAFGCDAALVRARYGKGFMGLVISMRD | |
| | SPPAEIIVVPADKTLARVGNPTDENAPAVLPGPPAGPRYRVFVLGAPTPADNGSAL | |
| | DALRRVAGYPEESTNYAQYMSRAYAEFLGEDPGSGTDARPSLFWRLAGLLASSGFA | |
| | FVNAAHAHDAIRLSDLLGFLAHSRVLAGLAARGAAGCAADSVFLNVSVLDPAARLR | |
| | LEARLGHLVAAILEREQSLAAHALGYQLAFVLDSPAAYGAVAPSAARLIDALYAEF | |
| | LGGRALTAPMVRRALFYATAVLRAPFLAGAPSAEQRERARRGLLITTALCTSDVAA | |
| | ATHADLRAALARTDHOKNLFWLPDHFSPCAASLRFDLAEGGFILDALAMATRSDIP | |
| | ADVMAQQTRGVASALTRWAHYNALIRAFVPEATHOCSGPSHNAEPRILVPITHNAS | |
| | YVVTHTPLPRGIGYKLTGVDVRRPLFITYLTATCEGHAREIEPKRLVRTENRRDLG | |
| | LVGAVFLRYTPAGEVMSVLLVDTDATQQQLAQGPVAGTPNVESSDVPSVALLLFPN | |
| | GTVIHLLAFDTLPIATIAPGFLAASALGVVMITAALAGILRVVRTCVPFLWRRE | |
| gL | MGFVCLFGLVVMGAWGAWGGSQATEYVLRSVIAKEVGDILRVPCMRTPADDVSWRY | 43 |
| | EAPSVIDYARIDGIFLRYHCPGLDTFLWDRHAQRAYLVNPFLFAAGFLEDLSHSVF | |
| | PADTQETTTRRALYKEIRDALGSRKQAVSHAPVRAGCVNFDYSRTRRCVGRRDLRP | |
| | ANTTSTWEPPVSSDDEASSQSKPLATQPPVLALSNAPPRRVSPTRGRRRHTRLRRN | |
| gL Asp | MAWGGSQATEYVLRSVIAKEVGDILRVPCMRTPADDVSWRYEAPSVIDYARIDGIF | 44 |
| | LRYHCPGLDTFLWDRHAQRAYLVNPFLFAAGFLEDLSHSVFPADTQETTTRRALYK | |
| | EIRDALGSRKQAVSHAPVRAGCVNEDYSRTRRCVGRRDLRPANTTSTWEPPVSSDD | |
| | EASSQSKPLATQPPVLALSNAPPRRVSPTRGRRRHTRLRRN | |
| gHgL link | MGFVCLFGLVVMGAWGAWGGSQATEYVLRSVIAKEVGDILRVPCMRTPADDVSWRY | 45 |
| | EAPSVIDYARIDGIFLRYHCPGLDTFLWDRHAQRAYLVNPFLFAAGFLEDLSHSVF | |
| | PADTQETTTRRALYKEIRDALGSRKQAVSHAPVRAGCVNFDYSRTRRCVGRRDLRP | |
| | ANTTSTWEPPVSSDDEASSQSKPLATQPPVLALSNAPPRRVSPTRGRRRHTRrRRr | |
| | gsggsgsggssgggsgsggsggsgsggrrrrrHDTYWTEQIDPWFLHGLGLARTYW | |
| | RDTNTGRLWLPNTPDASDPQRGRLAPPGELNLTTASVPMLRWYAERFCFVLVTTAE | |
| | FPRDPGOLLYIPKTYLLGRPRNASLPELPEAGPTSRPPAEVTOLKGLSHNPGASAL | |
| | LRSRAWVTFAAAPDREGLTFPRGDDGATERHPDGRRNAPPPGPPAGTPRHPTTNLS | |
| | IAHLHNASVTWLAARGLLRTPGRYVYLSPSASTWPVGVWTTGGLAFGCDAALVRAR | |
| | YGKGFMGLVISMRDSPPAEIIVVPADKTLARVGNPTDENAPAVLPGPPAGPRYRVF | |
| | VLGAPTPADNGSALDALRRVAGYPEESTNYAQYMSRAYAEFLGEDPGSGTDARPSL | |
| | FWRLAGLLASSGFAFVNAAHAHDAIRLSDLLGFLAHSRVLAGLAARGAAGCAADSV | |
| | FLNVSVLDPAARLRLEARLGHLVAAILEREQSLAAHALGYQLAFVLDSPAAYGAVA | |
| | PSAARLIDALYAEFLGGRALTAPMVRRALFYATAVLRAPFLAGAPSAEQRERARRG | |
| | LLITTALCTSDVAAATHADLRAALARTDHQKNLFWLPDHFSPCAASLRFDLAEGGE | |
| | ILDALAMATRSDIPADVMAQQTRGVASALTRWAHYNALIRAFVPEATHQCSGPSHN | |
| | AEPRILVPITHNASYVVTHTPLPRGIGYKLTGVDVRRPLFITYLTATCEGHAREIE | |
| | PKRLVRTENRRDLGLVGAVFLRYTPAGEVMSVLLVDTDATQQQLAQGPVAGTPNVF | |
| | SSDVPSVALLLFPNGTVIHLLAFDTLPIATIAPGFLAASALGVVMITAALAGILRV | |
| | VRTCVPFLWRRE | |
| ICPO | MEPRPGTSSRADPGPERPPROTPGTQPAAPHAWGMLNDMQWLASSDSEEETEVGIS | 46 |
| | DDDLHRDSTSEAGSTDTEMFEAGLMDAATPPARPPAERQGSPTPADAQGSCGGGPV | |
| | GEEEAEAGGGGDVNTPVAYLIVGVTASGSFSTIPIVNDPRTRVEAEAAVRAGTAVD | |
| | FIWTGNPRTAPRSLSLGGHTVRALSPTPPWPGTDDEDDDLADVDYVPPAPRRAPRR | |
| | GGGGAGATRGTSQPAATRPAPPGAPRSSSSGGAPLRAGVGSGSGGGPAVAAVVPRV | |
| | ASLPPAAGGGRAQARRVGEDAAAAEGRTPPAROPRAAQEPPIVISDSPPPSPRRPA | |
| | GPGPLSFVSSSSAQVSSGPGGGGLPQSSGRAARPRAAVAPRVRSPPRAAAAPVVSA | |
| | SADAAGPAPPAVPVDAHRAPRSRMTQAQTDTQAQSLGRAGATDARGSGGPGAEGGS | |
| | GPAASSSASSSAAPRSPLAPQGVGAKRAAPRRAPDSDSGDRGHGPLAPASAGAAPP | |
| | SASPSSQAAVAAASSSSASSSSASSSSASSSSASSSSASSSSASSSSASSSAGGAG | |
| | GSVASASGAGERRETSLGPRAAAPRGPRKCARKTRHAEGGPEPGARDPAPGLTRYL | |
| | PIAGVSSVVALAPYVNKTVTGDCLPVLDMETGHIGAYVVLVDQTGNVADLLRAAAP | |
| | AWSRRTLLPEHARNCVRPPDYPTPPASEWNSLWMTPVGNMLFDQGTLVGALDFHGL | |
| | RSRHPWSREQGAPAPAGDAPAGHGE | |
| ICPO | MNTPVAYLIVGVTASGSFSTIPIVNDPRTRVEAEAAVRAGTAVDFIWTGNPRTAPR | 47 |
| Variant | SLSLGGHTVRALSPTPPWPGTDDEDDDLADVDYVPPAPRRAPRRGGGGAGATRGTS | |
| | QPAATRPAPPGAPRSSSSGGAPLRAGVGSGSGGGPAVAAVVPRVASLPPAAGGGRA | |
| | QARRVGEDAAAAEGRTPPARQPRAAQEPPIVISDSPPPSPRRPAGPGPLSFVSSSS | |
| | AQVSSGPGGGGLPQSSGRAARPRAAVAPRVRSPPRAAAAPVVSASADAAGPAPPAV | |
| | PVDAHRAPRSRMTQAQTDTQAQSLGRAGATDARGSGGPGAEGGPGVPRGTNTPGAA | |
| | PHAAEGAAAGGGSGGGSPAPGLTRYLPIAGVSSVVALAPYVNKTVTGDCLPVLDME | |
| | TGHIGAYVVLVDQTGNVADLLRAAAPAWSRRTLLPEHARNCVRPPDYPTPPASEWN | |
| | SLWMTPVGNMLFDQGTLVGALDFHGLRSRHPWSREQGAP | |
| ICP4 | MSAEQQGRGAEVAMADEDGGRLRAAAETTGGPGSPDPADGPPPTPNPDRRPAARPG | 48 |
| | FGWHGGPEENEDEADASGEAVDEPAADGVVSPROLALLASMVDEAVRTIPSPPPER | |
| | DGAQEEAARSPSPPRTPSMRADYGEENAGRWVRGPETTSAVRGAYPDPMASLSPRP | |
| | PAPAARAPASAADHAAGGTLGADDEEAGVPARAPGAAPRPSPPRAEPAPARTPAAT | |
| | AGRLERRRARAAVAGRDATGRFTAGRPRRVELDADAASGAFYARYRDGYVSGEPWP | |
| | GAGPPPPGRVLYGGLGDSRPGLWGAPEAEEARARFEASGAPAPVWAPELGDAAQQY | |
| | ALITRLLYTPDAEAMGWLONPRVAPGDVALDOACFRISGAARNSSSFISGSVARAV | |
| | PHLGYAMAAGRFGWGLAHVAAAVAMSRRYDRAQKGFLLTSLRRAYAPLLARENAAL | |
| | TGARTPDDGGDANRHDGDDARGKPPLPSASPADERAVPAGYGAAGVLAALGRLSAA | |
| | PASAPAGARAEAGRVAVECLAACRGILEALAEGFDGDLAAVPGLAGARPAAPPRPG | |
| | PAGAAAPPHADAPRLRAWLRELRFVRDALVLMRLRGDLRVAGGSEAAVAAVRAVSL | |
| | VAGALGPALPRSPRLLSSDLLFQNQSLRPLLADTVAAADSLAAPASAPREAADAPR | |
| | PAAARPAALTRRPAEGPDPQGGWRRQPPGPSHTPAPSAAALEAYCAPRAVAELTDH | |
| | PLFPAPWRPALMFDPRALASLAARCAAPPPGGAPAAFGPLRASGPLRRAAAWMRQV | |
| | PDPEDVRVVILYSPLPGEDLAAGRAGGGPPPEWSAERGGLSCLLAALGNRLCGPAT | |
| | AAWAGNWTGAPDVSALGAQGVLLLSTRDLAFAGAVEFLGLLAGACDRRLIVVNAVR | |
| | AAAWPAAAPVVSRQHAYLACEVLPAVQCAVRWPAARDLRRTVLASGRVFGPGVFAR | |
| | VEAAHARLYPDAPPLRLCRGANVRYRVRTRFGPDTLVPMSPREYRRAVLPALDGRA | |
| | AASGAGDAMAPGAPDFCEDEAHSHRACARWGLGAPLRPVYVALGRDAVRGGPAELR | |
| | GPRREFCARALLEPDGDAPPLVLRDDADAGPPPQIRWASAAGRAGTVLAAAGGGVE | |
| | VVGTAAGLATPPRREPVDMDAELEDDDDGLEGE | |
| ICP4 | MRVLYGGLGDSRPGLWGAPEAEEARARFEASGAPAPVWAPELGDAAQQYALITRLL | 49 |
| Variant | YTPDAEAMGWLONPRVAPGDVALDQACFRISGAARNSSSFISGSVARAVPHLGYAM | |
| | AAGRFGWGLAHVAAAVAMSRRYDRAQKGFLLTSLRRAYAPLLARENAALTGARTPD | |
| | DGGDANRHDGDDARGKPPLPSAAASPADERAVPAGYGAAGVLAALGRLSAAPASAP | |
| | AGARAEAGRVAVECLAACRGILEALAEGFDGDLAAVPGLAGARPAAPPRPGPAGAA | |
| | APPHADAPRLRAWLRELRFVRDALVLMRLRGDLRVAGGSEAAVAAVRAVSLVAGAL | |
| | GPALPRSPRLLSSDLLFQNQSLGGGSGGGPPPGGAPAAFGPLRASGPLRRAAAWMR | |
| | QVPDPEDVRVVILYSPLPGEDLAAGRAGGGPPPEWSAERGGLSCLLAALGNRLCGP | |
| | ATAAWAGNWTGAPDVSALGAQGVLLLSTRDLAFAGAVEFLGLLAGACDRRLIVVNA | |
| | VRAADWPADGPVVSRQHAYLACEVLPAVOCAVRWPAARDLRRTVLASGRVFGPGVE | |
| | ARVEAAHARLYPDAPPLRLCRGANVRYRVRTRFGPDTLVPMSPREYRRAVLPALDG | |
| | RAAASGAGDAMAPGAPDFCEDEAHSHRACARWGLGAPLRPVYVALGRDAVRGGPAE | |
| | LRGPRREFCARALLE | |
| gB 2P#1 | MRGGGLVCALVVGALVAAVASAAPAAPRASGGVAATVAANGGPASQPPPVPSPATT | 50 |
| | KARKRKTKKPPKRPEATPPPDANATVAAGHATLRAHLREIKVENADAQFYVCPPPT | |
| | GATVVQFEQPRRCPTRPEGQNYTEGIAVVFKENIAPYKFKATMYYKDVTVSQVWFG | |
| | HRYSQFMGIFEDRAPVPFEEVIDKINAKGVCRSTAKYVRNNMETTAFHRDDHETDM | |
| | ELKPAKVATRTSRGWHTTDLKYNPSRVEAFHRYGTTVNCIVEEVDARSVYPYDEFV | |
| | LATGDFVYMSPFYGYREGSHTEHTSYAADRFKQVDGFYARDLTTKARATSPTTRNL | |
| | LTTPKFTVAWDWVPKRPAVCTMTKWQEVDEMLRAEYGGSFRESSDAISTTFTTNLT | |
| | QYSLSRVDLGDCIGRDAREAIDRMFARKYNATHIKVGQPQYYLATGGFLIAYQPLL | |
| | SNTLAELYVREYMREQDRKPRNATPAPLREAPSANASVERIKTTSSIEFARLOFTY | |
| | NHIQRHVNDMLGRPPVAWCELQNHELTLWNEARKLNPNAIASATVGRRVSARMLGD | |
| | VMAVSTCVPVAPDNVIVQNSMRVSSRPGTCYSRPLVSFRYEDQGPLIEGOLGENNE | |
| | LRLTRDALEPCTVGHRRYFIFGGGYVYFEEYAYSHQLSRADVTTVSTFIDLNITML | |
| | EDHEFVPLEVYTRHEIKDSGLLDYTEVQRRNQLHDLRFADIDTVIRADANAAMFAG | |
| | LCAFFEGMGDLGRAVGKVVMGVVGGVVSAVSGVSSFMSNPFGALAVGLLVLAGLVA | |
| | AFFAFRYVLQLQRNPMKALYPLTTKELKTSDPGGVGGEGEEGAEGGGFDEAKLAEA | |
| | REMIRYMALVSAMERTEHKARKKGTSALLSSKVTNMVLRKRNKARYSPLHNEDEAG | |
| | DEDEL | |
| gB 2P#2 | MRGGGLVCALVVGALVAAVASAAPAAPRASGGVAATVAANGGPASOPPPVPSPATT | 51 |
| | KARKRKTKKPPKRPEATPPPDANATVAAGHATLRAHLREIKVENADAQFYVCPPPT | |
| | GATVVQFEQPRRCPTRPEGQNYTEGIAVVFKENIAPYKFKATMYYKDVTVSQVWFG | |
| | HRYSQFMGIFEDRAPVPFEEVIDKINAKGVCRSTAKYVRNNMETTAFHRDDHETDM | |
| | ELKPAKVATRTSRGWHTTDLKYNPSRVEAFHRYGTTVNCIVEEVDARSVYPYDEFV | |
| | LATGDFVYMSPFYGYREGSHTEHTSYAADRFKQVDGFYARDLTTKARATSPTTRNL | |
| | LTTPKFTVAWDWVPKRPAVCTMTKWQEVDEMLRAEYGGSFRESSDAISTTFTTNLT | |
| | QYSLSRVDLGDCIGRDAREAIDRMFARKYNATHIKVGQPQYYLATGGFLIAYQPLL | |
| | SNTLAELYVREYMREQDRKPRNATPAPLREAPSANASVERIKTTSSIEFARLOFTY | |
| | NHIQRHVNDPPGRIAVAWCELQNHELTLWNEARKLNPNAIASATVGRRVSARMLGD | |
| | VMAVSTCVPVAPDNVIVONSMRVSSRPGTCYSRPLVSFRYEDQGPLIEGOLGENNE | |
| | LRLTRDALEPCTVGHRRYFIFGGGYVYFEEYAYSHQLSRADVTTVSTFIDLNITML | |
| | EDHEFVPLEVYTRHEIKDSGLLDYTEVQRRNQLHDLRFADIDTVIRADANAAMFAG | |
| | LCAFFEGMGDLGRAVGKVVMGVVGGVVSAVSGVSSFMSNPFGALAVGLLVLAGLVA | |
| | AFFAFRYVLQLQRNPMKALYPLTTKELKTSDPGGVGGEGEEGAEGGGFDEAKLAEA | |
| | REMIRYMALVSAMERTEHKARKKGTSALLSSKVTNMVLRKRNKARYSPLHNEDEAG | |
| | DEDEL | |
| gB 2P#3 | MRGGGLVCALVVGALVAAVASAAPAAPRASGGVAATVAANGGPASQPPPVPSPATT | 52 |
| | KARKRKTKKPPKRPEATPPPDANATVAAGHATLRAHLREIKVENADAQFYVCPPPT | |
| | GATVVQFEQPRRCPTRPEGQNYTEGIAVVFKENIAPYKFKATMYYKDVTVSQVWFG | |
| | HRYSQFMGIFEDRAPVPFEEVIDKINAKGVCRSTAKYVRNNMETTAFHRDDHETDM | |
| | ELKPAKVATRTSRGWHTTDLKYNPSRVEAFHRYGTTVNCIVEEVDARSVYPYDEFV | |
| | LATGDFVYMSPFYGYREGSHTEHTSYAADRFKQVDGFYARDLTTKARATSPTTRNL | |
| | LTTPKFTVAWDWVPKRPAVCTMTKWQEVDEMLRAEYGGSFRFSSDAISTTFTTNLT | |
| | QYSLSRVDLGDCIGRDAREAIDRMFARKYNATHIKVGQPQYYLATGGFLIAYQPLL | |
| | SNTLAELYVREYMREQDRKPRNATPAPLREAPSANASVERIKTTSSIEFARLOFTY | |
| | NHIQRPPNDMLGRIAVAWCELQNHELTLWNEARKLNPNAIASATVGRRVSARMLGD | |
| | VMAVSTCVPVAPDNVIVONSMRVSSRPGTCYSRPLVSFRYEDQGPLIEGOLGENNE | |
| | LRLTRDALEPCTVGHRRYFIFGGGYVYFEEYAYSHQLSRADVTTVSTFIDLNITML | |
| | EDHEFVPLEVYTRHEIKDSGLLDYTEVQRRNQLHDLRFADIDTVIRADANAAMFAG | |
| | LCAFFEGMGDLGRAVGKVVMGVVGGVVSAVSGVSSFMSNPFGALAVGLLVLAGLVA | |
| | AFFAFRYVLQLQRNPMKALYPLTTKELKTSDPGGVGGEGEEGAEGGGFDEAKLAEA | |
| | REMIRYMALVSAMERTEHKARKKGTSALLSSKVTNMVLRKRNKARYSPLHNEDEAG | |
| | DEDEL | |
| gB 2P#4 | MRGGGLVCALVVGALVAAVASAAPAAPRASGGVAATVAANGGPASOPPPVPSPATT | 53 |
| | KARKRKTKKPPKRPEATPPPDANATVAAGHATLRAHLREIKVENADAQFYVCPPPT | |
| | GATVVQFEQPRRCPTRPEGQNYTEGIAVVFKENIAPYKFKATMYYKDVTVSQVWFG | |
| | HRYSQFMGIFEDRAPVPFEEVIDKINAKGVCRSTAKYVRNNMETTAFHRDDHETDM | |
| | ELKPAKVATRTSRGWHTTDLKYNPSRVEAFHRYGTTVNCIVEEVDARSVYPYDEFV | |
| | LATGDFVYMSPFYGYREGSHTEHTSYAADRFKQVDGFYARDLTTKARATSPTTRNL | |
| | LTTPKFTVAWDWVPKRPAVCTMTKWQEVDEMLRAEYGGSFRFSSDAISTTFTTNLT | |
| | QYSLSRVDLGDCIGRDAREAIDRMFARKYNATHIKVGQPQYYLATGGFLIAYQPLL | |
| | SNTLAELYVREYMREQDRKPRNATPAPLREAPSANASVERIKTTSSIEFARLOFTY | |
| | NPPQRHVNDMLGRIAVAWCELONHELTLWNEARKLNPNAIASATVGRRVSARMLGD | |
| | VMAVSTCVPVAPDNVIVONSMRVSSRPGTCYSRPLVSFRYEDQGPLIEGOLGENNE | |
| | LRLTRDALEPCTVGHRRYFIFGGGYVYFEEYAYSHQLSRADVTTVSTFIDLNITML | |
| | EDHEFVPLEVYTRHEIKDSGLLDYTEVQRRNQLHDLRFADIDTVIRADANAAMFAG | |
| | LCAFFEGMGDLGRAVGKVVMGVVGGVVSAVSGVSSFMSNPFGALAVGLLVLAGLVA | |
| | AFFAFRYVLQLQRNPMKALYPLTTKELKTSDPGGVGGEGEEGAEGGGFDEAKLAEA | |
| | REMIRYMALVSAMERTEHKARKKGTSALLSSKVTNMVLRKRNKARYSPLHNEDEAG | |
| | DEDEL | |
| gB Act | MRGGGLVCALVVGALVAAVASAAPAAPRASGGVAATVAANGGPASOPPPVPSPATT | 54 |
| | KARKRKTKKPPKRPEATPPPDANATVAAGHATLRAHLREIKVENADAQFYVCPPPT | |
| | GATVVQFEQPRRCPTRPEGQNYTEGIAVVFKENIAPYKFKATMYYKDVTVSQVWFG | |
| | HRYSQFMGIFEDRAPVPFEEVIDKINAKGVCRSTAKYVRNNMETTAFHRDDHETDM | |
| | ELKPAKVATRTSRGWHTTDLKYNPSRVEAFHRYGTTVNCIVEEVDARSVYPYDEFV | |
| | LATGDFVYMSPFYGYREGSHTEHTSYAADRFKQVDGFYARDLTTKARATSPTTRNL | |
| | LTTPKFTVAWDWVPKRPAVCTMTKWQEVDEMLRAEYGGSFRFSSDAISTTFTTNLT | |
| | QYSLSRVDLGDCIGRDAREAIDRMFARKYNATHIKVGQPQYYLATGGFLIAYQPLL | |
| | SNTLAELYVREYMREQDRKPRNATPAPLREAPSANASVERIKTTSSIEFARLOFTY | |
| | NHIQRHVNDMLGRIAVAWCELQNHELTLWNEARKLNPNAIASATVGRRVSARMLGD | |
| | VMAVSTCVPVAPDNVIVONSMRVSSRPGTCYSRPLVSFRYEDQGPLIEGOLGENNE | |
| | LRLTRDALEPCTVGHRRYFIFGGGYVYFEEYAYSHQLSRADVTTVSTFIDLNITML | |
| | EDHEFVPLEVYTRHEIKDSGLLDYTEVQRRNQLHDLRFADIDTVIRADANAAMFAG | |
| | LCAFFEGMGDLGRAVGKVVMGVVGGVVSAVSGVSSFMSNPFGALAVGLLVLAGLVA | |
| | AFFAFRYVLQLQRN | |
| gB 2P#2Act | MRGGGLVCALVVGALVAAVASAAPAAPRASGGVAATVAANGGPASQPPPVPSPATT | 55 |
| | KARKRKTKKPPKRPEATPPPDANATVAAGHATLRAHLREIKVENADAQFYVCPPPT | |
| | GATVVQFEQPRRCPTRPEGQNYTEGIAVVFKENIAPYKFKATMYYKDVTVSQVWFG | |
| | HRYSQFMGIFEDRAPVPFEEVIDKINAKGVCRSTAKYVRNNMETTAFHRDDHETDM | |
| | ELKPAKVATRTSRGWHTTDLKYNPSRVEAFHRYGTTVNCIVEEVDARSVYPYDEFV | |
| | LATGDFVYMSPFYGYREGSHTEHTSYAADRFKQVDGFYARDLTTKARATSPTTRNL | |
| | LTTPKFTVAWDWVPKRPAVCTMTKWQEVDEMLRAEYGGSFRFSSDAISTTFTTNLT | |
| | QYSLSRVDLGDCIGRDAREAIDRMFARKYNATHIKVGQPQYYLATGGFLIAYQPLL | |
| | SNTLAELYVREYMREQDRKPRNATPAPLREAPSANASVERIKTTSSIEFARLOFTY | |
| | NHIQRHVNDPPGRIAVAWCELQNHELTLWNEARKLNPNAIASATVGRRVSARMLGD | |
| | VMAVSTCVPVAPDNVIVONSMRVSSRPGTCYSRPLVSFRYEDQGPLIEGOLGENNE | |
| | LRLTRDALEPCTVGHRRYFIFGGGYVYFEEYAYSHQLSRADVTTVSTFIDLNITML | |
| | EDHEFVPLEVYTRHEIKDSGLLDYTEVQRRNQLHDLRFADIDTVIRADANAAMFAG | |
| | LCAFFEGMGDLGRAVGKVVMGVVGGVVSAVSGVSSFMSNPFGALAVGLLVLAGLVA | |
| | AFFAFRYVLQLQRN | |
| gB 2P#4Act | MRGGGLVCALVVGALVAAVASAAPAAPRASGGVAATVAANGGPASQPPPVPSPATT | 56 |
| | KARKRKTKKPPKRPEATPPPDANATVAAGHATLRAHLREIKVENADAQFYVCPPPT | |
| | GATVVQFEQPRRCPTRPEGONYTEGIAVVFKENIAPYKFKATMYYKDVTVSQVWFG | |
| | HRYSQFMGIFEDRAPVPFEEVIDKINAKGVCRSTAKYVRNNMETTAFHRDDHETDM | |
| | ELKPAKVATRTSRGWHTTDLKYNPSRVEAFHRYGTTVNCIVEEVDARSVYPYDEFV | |
| | LATGDFVYMSPFYGYREGSHTEHTSYAADRFKQVDGFYARDLTTKARATSPTTRNL | |
| | LTTPKFTVAWDWVPKRPAVCTMTKWQEVDEMLRAEYGGSFRFSSDAISTTFTTNLT | |
| | QYSLSRVDLGDCIGRDAREAIDRMFARKYNATHIKVGQPQYYLATGGFLIAYQPLL | |
| | SNTLAELYVREYMREQDRKPRNATPAPLREAPSANASVERIKTTSSIEFARLOFTY | |
| | NPPQRHVNDMLGRIAVAWCELQNHELTLWNEARKLNPNAIASATVGRRVSARMLGD | |
| | VMAVSTCVPVAPDNVIVONSMRVSSRPGTCYSRPLVSFRYEDQGPLIEGOLGENNE | |
| | LRLTRDALEPCTVGHRRYFIFGGGYVYFEEYAYSHQLSRADVTTVSTFIDLNITML | |
| | EDHEFVPLEVYTRHEIKDSGLLDYTEVQRRNQLHDLRFADIDTVIRADANAAMFAG | |
| | LCAFFEGMGDLGRAVGKVVMGVVGGVVSAVSGVSSFMSNPFGALAVGLLVLAGLVA | |
| | AFFAFRYVLQLQRN | |
| gB | MRGGGLVCALVVGALVAAVASAAPAAPRASGGVAATVAANGGPASOPPPVPSPATT | 57 |
| 2P#3and4A | KARKRKTKKPPKRPEATPPPDANATVAAGHATLRAHLREIKVENADAQFYVCPPPT | |
| ct | GATVVQFEQPRRCPTRPEGQNYTEGIAVVFKENIAPYKFKATMYYKDVTVSQVWFG | |
| | HRYSQFMGIFEDRAPVPFEEVIDKINAKGVCRSTAKYVRNNMETTAFHRDDHETDM | |
| | ELKPAKVATRTSRGWHTTDLKYNPSRVEAFHRYGTTVNCIVEEVDARSVYPYDEFV | |
| | LATGDFVYMSPFYGYREGSHTEHTSYAADRFKQVDGFYARDLTTKARATSPTTRNL | |
| | LTTPKFTVAWDWVPKRPAVCTMTKWQEVDEMLRAEYGGSFRFSSDAISTTFTTNLT | |
| | QYSLSRVDLGDCIGRDAREAIDRMFARKYNATHIKVGQPQYYLATGGFLIAYQPLL | |
| | SNTLAELYVREYMREQDRKPRNATPAPLREAPSANASVERIKTTSSIEFARLOFTY | |
| | NPPQRPPNDMLGRIAVAWCELQNHELTLWNEARKLNPNAIASATVGRRVSARMLGD | |
| | VMAVSTCVPVAPDNVIVONSMRVSSRPGTCYSRPLVSFRYEDQGPLIEGOLGENNE | |
| | LRLTRDALEPCTVGHRRYFIFGGGYVYFEEYAYSHQLSRADVTTVSTFIDLNITML | |
| | EDHEFVPLEVYTRHEIKDSGLLDYTEVQRRNQLHDLRFADIDTVIRADANAAMFAG | |
| | LCAFFEGMGDLGRAVGKVVMGVVGGVVSAVSGVSSFMSNPFGALAVGLLVLAGLVA | |
| | AFFAFRYVLQLQRN | |
| gI | MPGRSLQGLAILGLWVCATGLVVRGPTVSLVSDSLVDAGAVGPQGFVEEDLRVEGE | 58 |
| | LHFVGAQVPHTNYYDGIIELFHYPLGNHCPRVVHVVTLTACPRRPAVAFTLCRSTH | |
| | HAHSPAYPTLELGLARQPLLRVRTATRDYAGLYVLRVWVGSATNASLFVLGVALSA | |
| | NGTFVYNGSDYGSCDPAQLPFSAPRLGPSSVYTPGASRPTPPRITTSPSSPRDPTP | |
| | APGDTGTPAPASGERAPPNSTRSASESRHRLTVAQVIQIAIPASIIAFVELGSCIC | |
| | FIHRCORRYRRPRGQIYNPGGVSCAVNEAAMARLGAELRSHPNTPPKPRRRSSSST | |
| | TMPSLTSIAEESEPGPVVLLSVSPRPRSGPTAPQEV | |
| sgI | MPGRSLQGLAILGLWVCATGLVVRGPTVSLVSDSLVDAGAVGPQGFVEEDLRVEGE | 59 |
| | LHFVGAQVPHTNYYDGIIELFHYPLGNHCPRVVHVVTLTACPRRPAVAFTLCRSTH | |
| | HAHSPAYPTLELGLARQPLLRVRTATRDYAGLYVLRVWVGSATNASRFVLGVALSA | |
| | NGTFVYNGSDYGSCDPAQLPFSAPRLGPSSVYTPGASRPTPPRTTTPPSSPRDPTP | |
| | APGDTGTPAPASGEIAPPNSTRSASESRHR | |
| gE Act | MARGAGLVFFVGVWVVSCLAAAPRTSWKRVTSGEDVVLLPAPAERTRAHKLLWAAE | 60 |
| | PLDACGPLRPSWVALWPPRRVLETVVDAACMRAPEPLAIAYSPPFPAGDEGLYSEL | |
| | AWRDRVAVVNESLVIYGALETDSGLYTLSVVGLSDEARQVASVVLVVEPAPVPTPT | |
| | PDDYDEEDDAGVSERTPVSVPPPTPPRRPPVAPPTHPRVIPEVSHVRGVTVHMETP | |
| | EAILFAPGETFGTNVSIHAIAHDDGPYAMDVVWMRFDVPSSCAEMRIYEACLYHPQ | |
| | LPECLSPADAPCAVSSWAYRLAVRSYAGCSRTTPPPRCFAEARMEPVPGLAWLAST | |
| | VNLEFQHASPQHAGLYLCVVYVDDHIHAWGHMTISTAAQYRNAVVEQHLPQRQPEP | |
| | VEPTRPHVRAPHPAPSARGPLRLGAVLGAALLLAALGLSAWA | |
| gI Act | MPGRSLQGLAILGLWVCATGLVVRGPTVSLVSDSLVDAGAVGPQGFVEEDLRVEGE | 61 |
| | LHFVGAQVPHTNYYDGIIELFHYPLGNHCPRVVHVVTLTACPRRPAVAFTLCRSTH | |
| | HAHSPAYPTLELGLARQPLLRVRTATRDYAGLYVLRVWVGSATNASRFVLGVALSA | |
| | NGTFVYNGSDYGSCDPAQLPFSAPRLGPSSVYTPGASRPTPPRTTTPPSSPRDPTP | |
| | APGDTGTPAPASGEIAPPNSTRSASESRHRLTVAQVIQIAIPASIIAFVELGSCIC | |
| | FIH | |
| gC Act | MALGRVGLAVGLWGLLWVGVVVVLANASPGRTITVGPRGNASNAAPSASPRNASAP | 62 |
| | RTTPTPPQPRKATKSKASTAKPAPPPKTGPPKTSSEPVRCNRHDPLARYGSRVOIR | |
| | CRFPNSTRTESRLQIWRYATATDAEIGTAPSLEEVMVNVSAPPGGOLVYDSAPNRT | |
| | DPHVIWAEGAGPGASPRLYSVVGPLGRORLIIEELTLETQGMYYWVWGRTDRPSAY | |
| | GTWVRVRVFRPPSLTIHPHAVLEGQPFKATCTAATYYPGNRAEFVWFEDGRRVFDP | |
| | AQIHTQTQENPDGFSTVSTVTSAAVGGQGPPRTFTCQLTWHRDSVSFSRRNASGTA | |
| | SVLPRPTITMEFTGDHAVCTAGCVPEGVTFAWFLGDDSSPAEKVAVASQTSCGRPG | |
| | TATIRSTLPVSYEQTEYICRLAGYPDGIPVLEHHGSHOPPPRDPTERQVIRAVEGA | |
| | GIGVAVLVAVVLAGTAVVYLT | |
| gC F327A | MALGRVGLAVGLWGLLWVGVVVVLANASPGRTITVGPRGNASNAAPSASPRNASAP | 63 |
| Act | RTTPTPPQPRKATKSKASTAKPAPPPKTGPPKTSSEPVRCNRHDPLARYGSRVQIR | |
| | CRFPNSTRTESRLQIWRYATATDAEIGTAPSLEEVMVNVSAPPGGQLVYDSAPNRT | |
| | DPHVIWAEGAGPGASPRLYSVVGPLGRORLIIEELTLETQGMYYWVWGRTDRPSAY | |
| | GTWVRVRVFRPPSLTIHPHAVLEGQPFKATCTAATYYPGNRAEFVWFEDGRRVEDP | |
| | AQIHTQTQENPDGFSTVSTVTSAAVGGQGPPRTFTCQLTWHRDSVSASRRNASGTA | |
| | SVLPRPTITMEFTGDHAVCTAGCVPEGVTFAWFLGDDSSPAEKVAVASQTSCGRPG | |
| | TATIRSTLPVSYEQTEYICRLAGYPDGIPVLEHHGSHQPPPRDPTERQVIRAVEGA | |
| | GIGVAVLVAVVLAGTAVVYLT | |
| gD Act | MGRLTSGVGTAALLVVAVGLRVVCAKYALADPSLKMADPNRFRGKNLPVLDQLTDP | 64 |
| | PGVKRVYHIQPSLEDPFQPPSIPITVYYAVLERACRSVLLHAPSEAPQIVRGASDE | |
| | ARKHTYNLTIAWYRMGDNCAIPITVMEYTECPYNKSLGVCPIRTOPRWSYYDSFSA | |
| | VSEDNLGFLMHAPAFETAGTYLRLVKINDWTEITQFILEHRARASCKYALPLRIPP | |
| | AACLTSKAYQQGVTVDSIGMLPRFIPENORTVALYSLKIAGWHGPKPPYTSTLLPP | |
| | ELSDTTNATQPELVPEDPEDSALLEDPAGTVSSQIPPNWHIPSIQDVAPHHAPAAP | |
| | SNPGLIIGALAGSTLAVLVIGGIAFWV | |
| gC | MALGRVGLAVGLWGLLWVGVVVVLANASPGRTITVGPRGNASNAAPSASPRNASAP | 65 |
| | RTTPTPPQPRKATKSKASTAKPAPPPKTGPPKTSSEPVRCNRHDPLARYGSRVQIR | |
| | CRFPNSTRTESRLQIWRYATATDAEIGTAPSLEEVMVNVSAPPGGOLVYDSAPNRT | |
| | DPHVIWAEGAGPGASPRLYSVVGPLGRORLIIEELTLETQGMYYWVWGRTDRPSAY | |
| | GTWVRVRVFRPPSLTIHPHAVLEGOPFKATCTAATYYPGNRAEFVWFEDGRRVEDP | |
| | AQIHTQTQENPDGFSTVSTVTSAAVGGQGPPRTFTCQLTWHRDSVSFSRRNASGTA | |
| | SVLPRPTITMEFTGDHAVCTAGCVPEGVTFAWFLGDDSSPAEKVAVASQTSCGRPG | |
| | TATIRSTLPVSYEQTEYICRLAGYPDGIPVLEHHGSHQPPPRDPTERQVIRAVEGA | |
| | GIGVAVLVAVVLAGTAVVYLTHASSVRYRRLR | |
| sgD | MGRLTSGVGTAALLVVAVGLRVVCAKYALADPSLKMADPNRFRGKNLPVLDQLTDP | 66 |
| | PGVKRVYHIQPSLEDPFQPPSIPITVYYAVLERACRSVLLHAPSEAPQIVRGASDE | |
| | ARKHTYNLTIAWYRMGDNCAIPITVMEYTECPYNKSLGVCPIRTOPRWSYYDSFSA | |
| | VSEDNLGFLMHAPAFETAGTYLRLVKINDWTEITQFILEHRARASCKYALPLRIPP | |
| | AACLTSKAYQQGVTVDSIGMLPRFIPENQRTVALYSLKIAGWHGPKPPYTSTLLPP | |
| | ELSDTTNATQPELVPEDPEDSALLEDPAGTVSSQIPPNWHIPSIQDVAPHHAPAAP | |
| | SNP | |
| gD gp120 | MGRLTSGVGTAALLVVAVGLRVVCAKYALADPSLKMADPNRFRGKNLPVLDQGVPV | 67 |
| | WKEATTTLFCASDAKAYDTEVHNVWATHACVPTDPNPQEVVLVNVTENFNMWKNDM | |
| | VEQMHEDIISLWDQSLKPCVKLTPLCVSLKCTDLKNDTNTNSSSGRMIMEKGEIKN | |
| | CSFNISTSIRGKVQKEYAFFYKLDIIPIDNDTTSYKLTSCNTSVITQACPKVSFEP | |
| | IPIHYCAPAGFAILKCNNKTFNGTGPCTNVSTVQCTHGIRPVVSTOLLINGSLAEE | |
| | EVVIRSVNFTDNAKTIIVQLNTSVEINCTRPNNNTRKRIRIQRGPGRAFVTIGKIG | |
| | NMRQAHCNISRAKWNNTLKQIASKLREQFGNNKTIIFKQSSGGDPEIVTHSENCGG | |
| | EFFYCNSTQLFNSTWFNSTWSTEGSNNTEGSDTITLPCRIKQIINMWQKVGKAMYA | |
| | PPISGQIRCSSNITGLLLTRDGGNSNNESEIFRPGGGDMRDNWRSELYKYKVVKIE | |
| | PLGVAPTKAKRRVVQGGGSGGGLIIGALAGSTLAVLVIGGIAFWVRRRAQMAPKRL | |
| | RLPHIRDDDAPPSHOPLFY | |
| sgD gp120 | MGRLTSGVGTAALLVVAVGLRVVCAKYALADPSLKMADPNRFRGKNLPVLDQGVPV | 68 |
| | WKEATTTLFCASDAKAYDTEVHNVWATHACVPTDPNPQEVVLVNVTENFNMWKNDM | |
| | VEQMHEDIISLWDQSLKPCVKLTPLCVSLKCTDLKNDTNTNSSSGRMIMEKGEIKN | |
| | CSFNISTSIRGKVQKEYAFFYKLDIIPIDNDTTSYKLISCNTSVITQACPKVSFEP | |
| | IPIHYCAPAGFAILKCNNKTENGTGPCTNVSTVOCTHGIRPVVSTOLLINGSLAEE | |
| | EVVIRSVNFTDNAKTIIVQLNTSVEINCTRPNNNTRKRIRIQRGPGRAFVTIGKIG | |
| | NMRQAHCNISRAKWNNTLKQIASKLREQFGNNKTIIFKQSSGGDPEIVTHSENCGG | |
| | EFFYCNSTQLENSTWENSTWSTEGSNNTEGSDTITLPCRIKQIINMWQKVGKAMYA | |
| | PPISGQIRCSSNITGLLLTRDGGNSNNESEIFRPGGGDMRDNWRSELYKYKVVKIE | |
| | PLGVAPTKAKRRVVQREKR | |
| SICPO | MVFTPQILGLMLEWISASRGNTPVAYLIVGVTASGSFSTIPIVNDPRTRVEAEAAV | 69 |
| Variant | RAGTAVDFIWTGNPRTAPRSLSLGGHTVRALSPTPPWPGTDDEDDDLADVDYVPPA | |
| | PRRAPRRGGGGAGATRGTSQPAATRPAPPGAPRSSSSGGAPLRAGVGSGSGGGPAV | |
| | AAVVPRVASLPPAAGGGRAQARRVGEDAAAAEGRTPPARQPRAAQEPPIVISDSPP | |
| | PSPRRPAGPGPLSFVSSSSAQVSSGPGGGGLPQSSGRAARPRAAVAPRVRSPPRAA | |
| | AAPVVSASADAAGPAPPAVPVDAHRAPRSRMTQAQTDTQAQSLGRAGATDARGSGG | |
| | PGAEGGPGVPRGTNTPGAAPHAAEGAAAGGGSGGGSPAPGLTRYLPIAGVSSVVAL | |
| | APYVNKTVTGDCLPVLDMETGHIGAYVVLVDQTGNVADLLRAAAPAWSRRTLLPEH | |
| | ARNCVRPPDYPTPPASEWNSLWMTPVGNMLFDQGTLVGALDFHGLRSRHPWSREQG | |
| | AP | |
| sICP4 | MVFTPQILGLMLEWISASRGRVLYGGLGDSRPGLWGAPEAEEARARFEASGAPAPV | 70 |
| Variant | WAPELGDAAQQYALITRLLYTPDAEAMGWLONPRVAPGDVALDQACFRISGAARNS | |
| | SSFISGSVARAVPHLGYAMAAGRFGWGLAHVAAAVAMSRRYDRAQKGFLLTSLRRA | |
| | YAPLLARENAALTGARTPDDGGDANRHDGDDARGKPPLPSAAASPADERAVPAGYG | |
| | AAGVLAALGRLSAAPASAPAGARAEAGRVAVECLAACRGILEALAEGFDGDLAAVP | |
| | GLAGARPAAPPRPGPAGAAAPPHADAPRLRAWLRELRFVRDALVLMRLRGDLRVAG | |
| | GSEAAVAAVRAVSLVAGALGPALPRSPRLLSSDLLFQNQSLGGGSGGGPPPGGAPA | |
| | AFGPLRASGPLRRAAAWMRQVPDPEDVRVVILYSPLPGEDLAAGRAGGGPPPEWSA | |
| | ERGGLSCLLAALGNRLCGPATAAWAGNWTGAPDVSALGAQGVLLLSTRDLAFAGAV | |
| | EFLGLLAGACDRRLIVVNAVRAADWPADGPVVSROHAYLACEVLPAVQCAVRWPAA | |
| | RDLRRTVLASGRVFGPGVFARVEAAHARLYPDAPPLRLCRGANVRYRVRTRFGPDT | |
| | LVPMSPREYRRAVLPALDGRAAASGAGDAMAPGAPDFCEDEAHSHRACARWGLGAP | |
| | LRPVYVALGRDAVRGGPAELRGPRREFCARALLE | |
| HSV-1 gB | MRQGAPARGRRWFVVWALLGLTLGVLVASAAPSSPGTPGVAAATQAANGGPATPAP | 111 |
| WT | PAPGAPPTGDPKPKKNRKPKPPKPPRPAGDNATVAAGHATLREHLRDIKAENTDAN | |
| (Accession | FYVCPPPTGATVVQFEQPRRCPTRPEGQNYTEGIAVVFKENIAPYKFKATMYYKDV | |
| No. | TVSQVWFGHRYSQFMGIFEDRAPVPFEEVIDKINAKGVCRSTAKYVRNNLETTAFH | |
| P10211) | RDDHETDMELKPANAATRTSRGWHTTDLKYNPSRVEAFHRYGTTVNCIVEEVDARS | |
| | VYPYDEFVLATGDFVYMSPFYGYREGSHTEHTSYAADRFKOVDGFYARDLTTKARA | |
| | TAPTTRNLLTTPKFTVAWDWVPKRPSVCTMTKWQEVDEMLRSEYGGSFRESSDAIS | |
| | TTFTTNLTEYPLSRVDLGDCIGKDARDAMDRIFARRYNATHIKVGQPQYYLANGGE | |
| | LIAYQPLLSNTLAELYVREHLREQSRKPPNPTPPPPGASANASVERIKTTSSIEFA | |
| | RLQFTYNHIQRHVNDMLGRVAIAWCELONHELTLWNEARKLNPNAIASATVGRRVS | |
| | ARMLGDVMAVSTCVPVAADNVIVONSMRISSRPGACYSRPLVSFRYEDQGPLVEGQ | |
| | LGENNELRLTRDAIEPCTVGHRRYFTFGGGYVYFEEYAYSHOLSRADITTVSTFID | |
| | LNITMLEDHEFVPLEVYTRHEIKDSGLLDYTEVQRRNOLHDLRFADIDTVIHADAN | |
| | AAMFAGLGAFFEGMGDLGRAVGKVVMGIVGGVVSAVSGVSSFMSNPFGALAVGLLV | |
| | LAGLAAAFFAFRYVMRLQSNPMKALYPLTTKELKNPTNPDASGEGEEGGDFDEAKL | |
| | AEAREMIRYMALVSAMERTEHKAKKKGTSALLSAKVTDMVMRKRRNTNYTQVPNKD | |
| | GDADEDDL | |
| HSV-2 gB | MRGGGLICALVVGALVAAVASAAPAAPAAPRASGGVAATVAANGGPASRPPPVPSP | 112 |
| WT | ATTKARKRKTKKPPKRPEATPPPDANATVAAGHATLRAHLREIKVENADAQFYVCP | |
| (Accession | PPTGATVVQFEQPRRCPTRPEGQNYTEGIAVVFKENIAPYKFKATMYYKDVTVSQV | |
| No. | WFGHRYSQFMGIFEDRAPVPFEEVIDKINAKGVCRSTAKYVRNNMETTAFHRDDHE | |
| P06763) | TDMELKPAKVATRTSRGWHTTDLKYNPSRVEAFHRYGTTVNCIVEEVDARSVYPYD | |
| | EFVLATGDFVYMSPFYGYREGSHTEHTSYAADRFKQVDGFYARDLTTKARATSPTT | |
| | RNLLTTPKFTVAWDWVPKRPAVCTMTKWQEVDEMLRAEYGGSFRFSSDAISTTETT | |
| | NLTQYSLSRVDLGDCIGRDAREAIDRMFARKYNATHIKVGQPQYYLATGGFLIAYQ | |
| | PLLSNTLAELYVREYMREQDRKPRNATPAPLREAPSANASVERIKTTSSIEFARLQ | |
| | FTYNHIQRHVNDMLGRIAVAWCELQNHELTLWNEARKLNPNAIASATVGRRVSARM | |
| | LGDVMAVSTCVPVAPDNVIVONSMRVSSRPGTCYSRPLVSFRYEDQGPLIEGOLGE | |
| | NNELRLTRDALEPCTVGHRRYFIFGGGYVYFEEYAYSHQLSRADVTTVSTFIDLNI | |
| | TMLEDHEFVPLEVYTRHEIKDSGLLDYTEVQRRNQLHDLRFADIDTVIRADANAAM | |
| | FAGLCAFFEGMGDLGRAVGKVVMGVVGGVVSAVSGVSSFMSNPFGALAVGLLVLAG | |
| | LVAAFFAFRYVLQLQRNPMKALYPLTTKELKTSDPGGVGGEGEEGAEGGGFDEAKL | |
| | AEAREMIRYMALVSAMERTEHKARKKGTSALLSSKVTNMVLRKRNKARYSPLHNED | |
| | EAGDEDEL | |
| HSV-1 gC | MAPGRVGLAVVLWSLLWLGAGVAGGSETASTGPTITAGAVTNASEAPTSGPPGSAA | 113 |
| WT | SPEVTPTSTPNPNNVTQNKTTPTEPASPPTTPKPTSTPKSPPTSTPDPKPKNNTTP | |
| (Accession | AKSGRPTKPPGPVWCDRRDPLARYGSRVOIRCRERNSTRMEFRLQIWRYSMGPSPP | |
| No. | IAPAPDLEEVLTNITAPPGGLLVYDSAPNLTDPHVLWAEGAGPGADPPLYSVTGPL | |
| Q8UZ70) | PTQRLIIGEVTPATQGMYYLAWGRMDSPHEYGTWVRVRMFRPPSLTLQPHAVMEGQ | |
| | PFKATCTAAAYYPRNPVEFVWFEDDRQVENPGQIDTQTHEHPDGFTTVSTVTSEAV | |
| | GGQVPPRTFTCOMTWHRDSVTFSRRNATGLALVLPRPTITMEFGVRHVACTAGCVP | |
| | EGVTFTWFLGDDPSPAAKSAVTAQESCDHPGLATVRSTLPISYDYSEYICRLTGYP | |
| | AGIPVLEHHGSHQPPPRDPTERQVIEAIEWVGIGIGVLAAGVLVVTAIVYVVRTSQ | |
| | SRQRHRR | |
| HSV-2 gC | MALGRVGLAVGLWGLLWVGVVVVLANASPGRTITVGPRGNASNAAPSASPRNASAP | 114 |
| WT | RTTPTPPQPRKATKSKASTAKPAPPPKTGPPKTSSEPVRCNRHDPLARYGSRVQIR | |
| (Accession | CRFPNSTRTEFRLOIWRYATATDAEIGTAPSLEEVMVNVSAPPGGQLVYDSAPNRT | |
| No. | DPHVIWAEGAGPGASPRLYSVVGPLGRORLIIEELTLETQGMYYWVWGRTDRPSAY | |
| Q89730) | GTWVRVRVFRPPSLTIHPHAVLEGQPFKATCTAATYYPGNRAEFVWFEDGRRVFDP | |
| | AQIHTQTQENPDGFSTVSTVTSAAVGGQGPPRTFTCQLTWHRDSVSFSRRNASGTA | |
| | SVLPRPTITMEFTGDHAVCTAGCVPEGVTFAWFLGDDSSPAEKVAVASQTSCGRPG | |
| | TATIRSTLPVSYEQTEYICRLAGYPDGIPVLEHHGSHOPPPRDPTERQVIRAVEGA | |
| | GIGVAVLVAVVLAGTAVVYLTHASSVRYRRLR | |
| HSV-1 gD | MGGAAARLGAVILFVVIVGLHGVRSKYALVDASLKMADPNRFRGKDLPVLDQLTDP | 115 |
| WT | PGVRRVYHIQAGLPDPFQPPSLPITVYYAVLERACRSVLLNAPSEAPQIVRGASED | |
| (Accession | VRKQPYNLTIAWFRMGGNCAIPITVMEYTECSYNKSLGACPIRTOPRWNYYDSFSA | |
| No. | VSEDNLGFLMHAPAFETAGTYLRLVKINDWTEITQFILEHRAKGSCKYALPLRIPP | |
| Q69091) | SACLSPQAYQQGVTVDSIGMLPRFIPENQRTVAVYSLKIAGWHGPKAPYTSTLLPP | |
| | ELSETPNATQPELAPEDPEDSALLEDPVGTVAPQIPPNWHIPSIQDAATPYHPPAT | |
| | PNNMGLIAGAVGGSLLAALVICGIVYWMRRHTQKAPKRIRLPHIREDDQPSSHQPL | |
| | FY | |
| HSV-2 gD | MGRLTSGVGTAALLVVAVGLRVVCAKYALADPSLKMADPNRFRGKNLPVLDRLTDP | 116 |
| WT | PGVKRVYHIQPSLEDPFQPPSIPITVYYAVLERACRSVLLHAPSEAPQIVRGASDE | |
| (Accession | ARKHTYNLTIAWYRMGDNCAIPITVMEYTECPYNKSLGVCPIRTOPRWSYYDSFSA | |
| No. | VSEDNLGFLMHAPAFETAGTYLRLVKINDWTEITOFILEHRARASCKYALPLRIPP | |
| P03172) | AACLTSKAYQQGVTVDSIGMLPRFIPENQRTVALYSLKIAGWHGPKPPYTSTLLPP | |
| | ELSDTTNATQPELVPEDPEDSALLEDPAGTVSSQIPPNWHIPSIQDVAPHHAPAAP | |
| | SNPGLIIGALAGSTLAVLVIGGIAFWVRRRAQMAPKRLRLPHIRDDDAPPSHOPLF | |
| | Y | |
| HSV-1 | MEPRPGASTRRPEGRPQREPAPDVWVFPCDRDLPDSSDSEAETEVGGRGDADHHDD | 117 |
| ICPO WT | DSASEADSTDTELFETGLLGPQGVDGGAVSGGSPPREEDPGSCGGAPPREDGGSDE | |
| (Accession | GDVCAVCTDEIAPHLRCDTFPCMHRFCIPCMKTWMQLRNTCPLCNAKLVYLIVGVT | |
| No. | PSGSFSTIPIVNDPQTRMEAEEAVRAGTAVDFIWTGNQRFAPRYLTLGGHTVRALS | |
| P08393) | PTHPEPTIDEDDDDLDDADYVPPAPRRTPRAPPRRGAAAPPVTGGASHAAPQPAAA | |
| | RTAPPSAPIGPHGSSNTNTTTNSSGGGGSRQSRAAAPRGASGPSGGVGVGVGVVEA | |
| | EAGRPRGRTGPLVNRPAPLANNRDPIVISDSPPASPHRPPAAPMPGSAPRPGPPAS | |
| | AAASGPARPRAAVAPCVRAPPPGPGPRAPAPGAEPAARPADARRVPQSHSSLAQAA | |
| | NOEQSLCRARATVARGSGGPGVEGGHGPSRGAAPSGAAPLPSAASVEQEAAVRPRK | |
| | RRGSGQENPSPQSTRPPLAPAGAKRAATHPPSDSGPGGRGOGGPGTPLISSAASAS | |
| | SSSASSSSAPTPAGAASSAAGAASSSASASSGGAVGALGGRQEETSLGPRAASGPR | |
| | GPRKCARKTRHAETSGAVPAGGLTRYLPISGVSSVVALSPYVNKTITGDCLPILDM | |
| | ETGNIGAYVVLVDQTGNMATRLRAAVPGWSRRTLLPETAGNHVMPPEYPTAPASEW | |
| | NSLWMTPVGNMLFDQGTLVGALDERSLRSRHPWSGEQGASTRDEGKQ | |
| HSV-2 | MEPRPGTSSRADPGPERPPRQTPGTQPAAPHAWGMLNDMQWLASSDSEEETEVGIS | 118 |
| ICPO WT | DDDLHRDSTSEAGSTDTEMFEAGLMDAATPPARPPAEROGSPTPADAQGSCGGGPV | |
| (Accession | GEEEAEAGGGGDVCAVCTDEIAPPLRCQSFPCLHPFCIPCMKTWIPLRNTCPLCNT | |
| No. | PVAYLIVGVTASGSFSTIPIVNDPRTRVEAEAAVRAGTAVDFIWTGNPRTAPRSLS | |
| P28284) | LGGHTVRALSPTPPWPGTDDEDDDLADVDYVPPAPRRAPRRGGGGAGATRGTSQPA | |
| | ATRPAPPGAPRSSSSGGAPLRAGVGSGSGGGPAVAAVVPRVASLPPAAGGGRAQAR | |
| | RVGEDAAAAEGRTPPARQPRAAQEPPIVISDSPPPSPRRPAGPGPLSFVSSSSAQV | |
| | SSGPGGGGLPQSSGRAARPRAAVAPRVRSPPRAAAAPVVSASADAAGPAPPAVPVD | |
| | AHRAPRSRMTQAQTDTQAQSLGRAGATDARGSGGPGAEGGPGVPRGTNTPGAAPHA | |
| | AEGAAARPRKRRGSDSGPAASSSASSSAAPRSPLAPQGVGAKRAAPRRAPDSDSGD | |
| | RGHGPLAPASAGAAPPSASPSSQAAVAAASSSSASSSSASSSSASSSSASSSSASS | |
| | SSASSSSASSSAGGAGGSVASASGAGERRETSLGPRAAAPRGPRKCARKTRHAEGG | |
| | PEPGARDPAPGLTRYLPIAGVSSVVALAPYVNKTVTGDCLPVLDMETGHIGAYVVL | |
| | VDQTGNVADLLRAAAPAWSRRTLLPEHARNCVRPPDYPTPPASEWNSLWMTPVGNM | |
| | LFDQGTLVGALDFHGLRSRHPWSREQGAPAPAGDAPAGHGE | |
| HSV-1 | MASENKQRPGSPGPTDGPPPTPSPDRDERGALGWGAETEEGGDDPDHDPDHPHDLD | 119 |
| ICP4 WT | DARRDGRAPAAGTDAGEDAGDAVSPRQLALLASMVEEAVRTIPTPDPAASPPRTPA | |
| (Accession | FRADDDDGDEYDDAADAAGDRAPARGREREAPLRGAYPDPTDRLSPRPPAQPPRRR | |
| No. | RHGRWRPSASSTSSDSGSSSSSSASSSSSSSDEDEDDDGNDAADHAREARAVGRGP | |
| P08392) | SSAAPAAPGRTPPPPGPPPLSEAAPKPRAAARTPAASAGRIERRRARAAVAGRDAT | |
| | GRFTAGQPRRVELDADATSGAFYARYRDGYVSGEPWPGAGPPPPGRVLYGGLGDSR | |
| | PGLWGAPEAEEARRRFEASGAPAAVWAPELGDAAQQYALITRLLYTPDAEAMGWLQ | |
| | NPRVVPGDVALDQACFRISGAARNSSSFITGSVARAVPHLGYAMAAGREGWGLAHA | |
| | AAAVAMSRRYDRAQKGFLLTSLRRAYAPLLARENAALTGAAGSPGAGADDEGVAAV | |
| | AAAAPGERAVPAGYGAAGILAALGRLSAAPASPAGGDDPDAARHADADDDAGRRAQ | |
| | AGRVAVECLAACRGILEALAEGFDGDLAAVPGLAGARPASPPRPEGPAGPASPPPP | |
| | HADAPRLRAWLRELRFVRDALVLMRLRGDLRVAGGSEAAVAAVRAVSLVAGALGPA | |
| | LPRDPRLPSSAAAAAADLLFDNOSLRPLLAAAASAPDAADALAAAAASAAPREGRK | |
| | RKSPGPARPPGGGGPRPPKTKKSGADAPGSDARAPLPAPAPPSTPPGPEPAPAQPA | |
| | APRAAAAQARPRPVAVSRRPAEGPDPLGGWRROPPGPSHTAAPAAAALEAYCSPRA | |
| | VAELTDHPLFPVPWRPALMFDPRALASIAARCAGPAPAAQAACGGGDDDDNPHPHG | |
| | AAGGRLFGPLRASGPLRRMAAWMRQIPDPEDVRVVVLYSPLPGEDLAGGGASGGPP | |
| | EWSAERGGLSCLLAALANRLCGPDTAAWAGNWTGAPDVSALGAQGVLLLSTRDLAF | |
| | AGAVEFLGLLASAGDRRLIVVNTVRACDWPADGPAVSRQHAYLACELLPAVQCAVR | |
| | WPAARDLRRTVLASGRVFGPGVFARVEAAHARLYPDAPPLRLCRGGNVRYRVRTRE | |
| | GPDTPVPMSPREYRRAVLPALDGRAAASGTTDAMAPGAPDFCEEEAHSHAACARWG | |
| | LGAPLRPVYVALGREAVRAGPARWRGPRRDFCARALLEPDDDAPPLVLRGDDDGPG | |
| | ALPPAPPGIRWASATGRSGTVLAAAGAVEVLGAEAGLATPPRREVVDWEGAWDEDD | |
| | GGAFEGDGVL | |
| HSV-2 | MSAEQRKKKKTTTTTQGRGAEVAMADEDGGRLRAAAETTGGPGSPDPADGPPPTPN | 120 |
| ICP4 WT | PDRRPAARPGFGWHGGPEENEDEADDAAADADADEAAPASGEAVDEPAADGVVSPR | |
| (Accession | QLALLASMVDEAVRTIPSPPPERDGAQEEAARSPSPPRTPSMRADYGEENDDDDDD | |
| No. | DDDDDRDAGRWVRGPETTSAVRGAYPDPMASLSPRPPAPRRHHHHHHHRRRRAPRR | |
| P90493) | RSAASDSSKSGSSSSASSASSSASSSSSASASSSDDDDDDDAARAPASAADHAAGG | |
| | TLGADDEEAGVPARAPGAAPRPSPPRAEPAPARTPAATAGRLERRRARAAVAGRDA | |
| | TGRFTAGRPRRVELDADAASGAFYARYRDGYVSGEPWPGAGPPPPGRVLYGGLGDS | |
| | RPGLWGAPEAEEARARFEASGAPAPVWAPELGDAAQQYALITRLLYTPDAEAMGWL | |
| | QNPRVAPGDVALDQACFRISGAARNSSSFISGSVARAVPHLGYAMAAGRFGWGLAH | |
| | VAAAVAMSRRYDRAQKGFLLTSLRRAYAPLLARENAALTGARTPDDGGDANRHDGD | |
| | DARGKPAAAAAPLPSAAASPADERAVPAGYGAAGVLAALGRLSAAPASAPAGADDD | |
| | DDDDGAGGGGGGRRAEAGRVAVECLAACRGILEALAEGFDGDLAAVPGLAGARPAA | |
| | PPRPGPAGAAAPPHADAPRLRAWLRELRFVRDALVLMRLRGDLRVAGGSEAAVAAV | |
| | RAVSLVAGALGPALPRSPRLLSSAAAAAADLLFONOSLRPLLADTVAAADSLAAPA | |
| | SAPREARKRKSPAPARAPPGGAPRPPKKSRADAPRPAAAPPAGAAPPAPPTPPPRP | |
| | PRPAALTRRPAEGPDPOGGWRROPPGPSHTPAPSAAALEAYCAPRAVAELTDHPLF | |
| | PAPWRPALMEDPRALASLAARCAAPPPGGAPAAFGPLRASGPLRRAAAWMRQVPDP | |
| | EDVRVVILYSPLPGEDLAAGRAGGGPPPEWSAERGGLSCLLAALGNRLCGPATAAW | |
| | AGNWTGAPDVSALGAQGVLLLSTRDLAFAGAVEFLGLLAGACDRRLIVVNAVRAAD | |
| | WPADGPVVSRQHAYLACEVLPAVQCAVRWPAARDLRRTVLASGRVFGPGVFARVEA | |
| | AHARLYPDAPPLRLCRGANVRYRVRTRFGPDTLVPMSPREYRRAVLPALDGRAAAS | |
| | GAGDAMAPGAPDFCEDEAHSHRACARWGLGAPLRPVYVALGRDAVRGGPAELRGPR | |
| | REFCARALLEPDGDAPPLVLRDDADAGPPPQIRWASAAGRAGTVLAAAGGGVEVVG | |
| | TAAGLATPPRREPVDMDAELEDDDDGLEGE | |

“Δct” indicates a deletion of the cytoplasmic tail.
“Δsp” indicates a deletion of the signal peptide.

Some aspects of the present disclosure provide a herpes simplex virus (HSV) protein comprising an amino acid sequence having at least 85%, at least 90%, at least 95%, at least 96%, at least 97%, at least 98%, at least 99%, or 100% identity to the amino acid sequence of any one of SEQ ID NOs: 36-70.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 36.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 37.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 38.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 39.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 40.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 41.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 42.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 43.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 44.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 45.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 46.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 47.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 48.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 49.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 50.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 51.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 52.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 53.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 54.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 55.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 56.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 57.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 58.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 59.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 60.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 61.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 62.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 63.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 64.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 65.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 66.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 67.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 68.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 69.

In some embodiments, an HSV protein comprises an amino acid sequence having at least 85%, at least 90%, at least 95%, at least 96%, at least 97%, at least 98%, at least 99%, or 100% identity to the amino acid sequence of SEQ ID NO: 70.

### Signal Peptides

In some embodiments, an RNA (e.g., mRNA) has an ORF that encodes a signal peptide fused to the HSV protein. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than HSV proteins in nature) are known in the art and can be tested for desired properties and then incorporated into a nucleic acid of the disclosure.

In some embodiments, an RNA (e.g., mRNA) comprises an open reading frame that encodes an HSV protein fused to a signal peptide comprising an amino acid sequence that has at least 50%, at least 60%, at least 70%, at least 75%, at least 80%, at least 85%, at least 90%, or at least 95% identity to the amino acid sequence of any one of the sequences provided herein. See, e.g., SEQ ID NOs: 71-75, which are reproduced below in Table 3. In some embodiments, an mRNA comprises an open reading frame that encodes an HSV protein including an endogenous signal peptide of the wild-type HSV protein (e.g., an mRNA encoding a (wild-type or modified) HSV gB encodes an HSV gB signal peptide).

TABLE 3
Signal Peptides

| Description | Sequence | SEQ ID NO: |
|---|---|---|
| HulgGk signal peptide | METPAQLLFLLLLWLPDTTG | 71 |
| IgE heavy chain epsilon −1 signal | MDWTWILFLVAAATR VHS | 72 |
| peptide | | |
| Japanese encephalitis PRM signal | MLGSNSGQRVVFTILLLLVAPAYS | 73 |
| sequence | | |
| VSVg protein signal sequence | MKCLLYLAFLFIGVNCA | 74 |
| Japanese encephalitis JEV signal | MWLVSLAIVTACAGA | 75 |
| sequence | | |

### Fusion Proteins

In some embodiments, an RNA (e.g., mRNA) encodes a fusion protein. Thus, an encoded protein may include two or more proteins (e.g., protein and/or protein fragment) joined together with or without a linker. Fusion proteins, in some embodiments, retain the functional property of each independent (nonfusion) protein.

In some embodiments, a fusion protein comprises 2, 3, 4, 5, 6, 7, 8, 9, or 10 of the following HSV proteins: gB, gC, gD, gE, gH, gL, gI, ICP0, and ICP4. Exemplary fusion proteins of the disclosure are provided in Table 2. For example, in some embodiments, an RNA (e.g., mRNA) encodes a protein that has at least 50%, at least 60%, at least 70%, at least 75%, at least 80%, at least 85%, at least 90%, or at least 95% identity to a sequence selected from SEQ ID NOs:36-70. In some embodiments, an RNA (e.g., mRNA) encodes a protein that is 80%, 81%, 82%, 83%, 84%, 85%, 86%, 87%, 88%, 89%, 90%, 91%, 92%, 93%, 94%, 95%, 96%, 97%, 98%, 99% or identical to a sequence selected from SEQ ID NOs: 36-70. In some embodiments, the RNA (e.g., mRNA) vaccines of the disclosure comprise an ORF that has at least 50%, at least 60%, at least 70%, at least 75%, at least 80%, at least 85%, at least 90%, or at least 95% identity to a sequence selected from SEQ ID NOs: 1-35. In some embodiments, the RNA (e.g., mRNA) vaccines of the disclosure comprise an ORF that has 50%, 60%, 70%, 75%, 80%, 85%, 90%, or 95% identity to a sequence selected from SEQ ID NOs: 1-35.

### Linkers and Cleavable Peptides

In some embodiments, an RNA (e.g., mRNA) that encodes a fusion protein further encodes a linker located between at least one or each domain of the fusion protein. The linker may be, for example, a cleavable linker or protease-sensitive linker. In some embodiments, the linker is selected from the group consisting of F2A linker, P2A linker, T2A linker, E2A linker, and combinations thereof (see, e.g., WO 2017/127750). This family of self-cleaving peptide linkers, referred to as 2A peptides, has been described in the art (see, e.g., Kim, J. H. et al. PLoS ONE 2011; 6:e18556). In some embodiments, the linker is an F2A linker.

In some embodiments, the linker is a GS linker. GS linkers are polypeptide linkers that include glycine and serine amino acids repeats. They comprise flexible and hydrophilic residues and can be used to perform fusion of protein subunits without interfering in the folding and function of the protein domains, and without formation of secondary structures. In some embodiments, an RNA (e.g., mRNA) encodes a fusion protein that comprises a GS linker that is 3 to 20 amino acids long. For example, the GS linker may have a length of (or have a length of at least) 3, 4, 5, 6, 7, 8, 9, 10, 11, 12, 13, 14, 15, 16, 17, 18, 19, or 20 amino acids. In some embodiments, a GS linker is (or is at least) 15 amino acids long (e.g., GGSGGSGGSGGSGGG (SEQ ID NO: 76)). In some embodiments, a GS linker is (or is at least) 8 amino acids long (e.g., GGGSGGGS (SEQ ID NO: 77)). In some embodiments, a GS linker is (or is at least) 7 amino acids long (e.g., GGGSGGG (SEQ ID NO: 78)). In some embodiments, a GS linker comprises the amino acid sequence GGGSGG (SEQ ID NO: 82). In some embodiments, a GS linker is (or is at least) 4 amino acid long (e.g., GGGS (SEQ ID NO: 79)). In some embodiments, the GS linker comprises (GGGS)n (SEQ ID NO: 80), where n is any integer from 1-5. In some embodiments, a GS linker is (or is at least) 4 amino acid long (e.g., GSGG (SEQ ID NO: 81)). In some embodiments, the GS linker comprises (GSGG)n (SEQ ID NO: 80), where n is any integer from 1-5. In some embodiments, a linker is a glycine linker, for example having a length of (or a length of at least) 3 amino acids (e.g., GGG). In some embodiments, a protein encoded by an RNA (e.g., mRNA) includes two or more linkers, which may be the same or different from each other.

The skilled artisan will appreciate that other art-recognized linkers may be suitable for use in the constructs of the disclosure (e.g., encoded by the nucleic acids of the disclosure). The skilled artisan will likewise appreciate that other polycistronic constructs (RNA (e.g., mRNA) encoding more than one protein separately within the same molecule) may be suitable for use as provided herein.

### Nucleic Acids Encoding Herpes Simplex Virus Proteins

Nucleic acids comprise a polymer of nucleotides (nucleotide monomers). Thus, nucleic acids are also referred to as polynucleotides. Nucleic acids may be or may include, for example, deoxyribonucleic acid (DNA), ribonucleic acid (RNA), threose nucleic acid (TNA), glycol nucleic acid (GNA), peptide nucleic acid (PNA), locked nucleic acid (LNA, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acid (ENA), cyclohexenyl nucleic acid (CeNA) and/or chimeras and/or combinations thereof.

RNA (e.g., mRNA) of the present disclosure comprises an open reading frame (ORF) encoding a HSV protein. In some embodiments, the RNA (e.g., mRNA) further comprises a 5′ untranslated region (UTR), 3′ UTR, a poly(A) tail and/or a 5′ cap analog.

### Messenger RNA

Messenger RNA (mRNA) is RNA that encodes a (at least one) protein (a naturally-occurring, non-naturally-occurring, or modified polymer of amino acids) and can be translated to produce the encoded protein in vitro, in vivo, in situ, or ex vivo. It is understood that mRNA is not self-amplifying RNA (saRNA) (see, e.g., Bloom K et al. Gene Therapy 2021; 28: 117-129 for a comparison of mRNA and saRNA). saRNAs include alphavirus replicase sequences that encode an RNA-dependent RNA polymerase. mRNA does not include alphavirus replicase sequences.

The skilled artisan will appreciate that, except where otherwise noted, nucleic acid sequences set forth in the instant application may recite “T”s in a representative DNA sequence but where the sequence represents mRNA, the “T”s would be substituted for “U”s. Thus, any of the DNAs disclosed and identified by a particular sequence identification number herein also disclose the corresponding mRNA sequence complementary to the DNA, where each “T” of the DNA sequence is substituted with “U.”

Naturally-occurring eukaryotic mRNA molecules can contain stabilizing elements, including, but not limited to, UTRs at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

Exemplary sequences of mRNA that encode HSV proteins of the present disclosure are provided in Table 1. In some embodiments, the mRNA comprises an ORF that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or identical to a sequence selected from SEQ ID NOs: 1-35. In some embodiments, the mRNA comprises a nucleotide sequence that is 80%, 81%, 82%, 83%, 84%, 85%, 86%, 87%, 88%, 89%, 90%, 91%, 92%, 93%, 94%, 95%, 96%, 97%, 98%, 99% or identical to a sequence selected from SEQ ID NOs: 1-35.

### Untranslated Regions (UTRs)

The mRNAs of the present disclosure may comprise one or more regions or parts which act or function as an untranslated region. A “5′ untranslated region” (UTR) refers to a region of an mRNA that is directly upstream (i.e., 5′) from the start codon (i.e., the first codon of an mRNA transcript translated by a ribosome) that does not encode a polypeptide. A “3′ untranslated region” (UTR) refers to a region of an mRNA that is directly downstream (i.e., 3′) from the stop codon (i.e., the codon of an mRNA transcript that signals a termination of translation) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

Where mRNAs are designed to encode a (at least one) HSV protein, the mRNA may comprise a 5′ UTR and/or 3′ UTR. UTRs of an mRNA are transcribed but not translated. In mRNA, the 5′ UTR starts at the transcription start site and continues to the start codon but does not include the start codon; the 3′ UTR starts immediately following the stop codon and continues until the transcriptional termination signal. There is a growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′ UTR and 3′ UTR sequences are known.

It should also be understood that the mRNA of the present disclosure may include any 5′ UTR and/or any 3′ UTR. Exemplary UTR sequences include SEQ ID NOs: 83-93; however, other UTR sequences may be used or exchanged for any of the UTR sequences described herein.

In some embodiments, a 5′ UTR of the present disclosure comprises a sequence selected from:

(SEQ ID NO: 83)
GGGAAAUAAGAGAGAAAAGAAGAGUAAGAAGAAAUAUAAGAGCCACC,
(SEQ ID NO: 84)
GGGAAAUAAGAGAGAAAAGAAGAGUAAGAAGAAAUAUAAGACCCCGGCGC
CGCCACC,
(SEQ ID NO: 85)
GAGGAAAUCGCAAAAUUUGCUCUUCGCGUUAGAUUUCUUUUAGUUUUCUC
GCAACUAGCAAGCUUUUUGUUCUCGCC,
and
(SEQ ID NO: 109)
GGAAAUCGCAAAAUUUGCUCUUCGCGUUAGAUUUCUUUUAGUUUUCUCGC
AACUAGCAAGCUUUUUGUUCUCGCC.

In some embodiments, a 3′ UTR of the present disclosure comprises a sequence selected from

(SEQ ID NO: 86)
UGAUAAUAGGCUGGAGCCUCGGUGGCCAUGCUUCUUGCCCCUUGGGCCUC
CCCCCAGCCCCUCCUCCCCUUCCUGCACCCGUACCCCCGUGGUCUUUGAA
UAAAGUCUGAGUGGGCGGC,
(SEQ ID NO: 87)
UGAUAAUAGGCUGGAGCCUCGGUGGCCUAGCUUCUUGCCCCUUGGGCCUC
CCCCCAGCCCCUCCUCCCCUUCCUGCACCCGUACCCCCGUGGUCUUUGAA
UAAAGUCUGAGUGGGCGGC,
(SEQ ID NO: 88)
UAAAGCUCCCCGGGGGCCUCGGUGGCCUAGCUUCUUGCCCCUUGGGCCUC
CCCCCAGCCCCUCCUCCCCUUCCUGCAGGAGAUUGAGUGUAGUGACUAGU
GGUCUUUGAAUAAAGUCUGAGUGGGCGGC,
(SEQ ID NO: 89)
UAAAGCUCCCCGGGGGCCUCGGUGGCCUAGCUUCUUGCCCCUUGGGCCUC
CCCCCAGCCCCUCCUCCCCUUCCUGCAGGAUUGAGACUACGGGUGGUCUU
UGAAUAAAGUCUGAGUGGGGGC,
(SEQ ID NO: 90)
UAAAGCUCCCCGGGGGCCUCGGUGGCCUAGCUUCUUGCCCCUUGGGCCUC
CCCCCAGCCCCUCCUCCCCUUCCUGCAGCAUAGACACUACGUGGUCUUUG
AAUAAAGUCUGAGUGGGCGGC,
(SEQ ID NO: 91)
UAAAGCUCCCCGGGGGCCUCGGUGGCCUAGCUUCUUGCCCCUUGGGCCUC
CCCCCAGCCCCUCCUCCCCUUCCUGCAGGAGAUUGAGUGUAGUGGUGGUC
UUUGAAUAAAGUCUGAGUGGGCGGC,
(SEQ ID NO: 92)
UAAAGCUCCCCGGGGGCCUCGGUGGCCUAGCUUCUUGCCCCUUGGGCCUC
CCCCCAGCCCCUCCUCCCCUUCCUGCAGGAGAUUGAGUGUAGUGACGUGG
UCUUUGAAUAAAGUCUGAGUGGGCGGC,
(SEQ ID NO: 93)
UAAAGCUCCCCGGGGGCCUCGGUGGCCUAGCUUCUUGCCCCUUGGGCCUC
CCCCCAGCCCCUCCUCCCCUUCCUGCAGUGGUCUUUGAAUAAAGUCUGAG
UGGGCGGC,
and
(SEQ ID NO: 110)
UAAAGCUCCCCGGGGGCCUCGGUGGCCUAGCUUCUUGCCCCUUGGGCCUC
CCCCCAGCCCCUCCUCCCCUUCCUGCACCCGUACCCCCGUGGUCUUUGAA
UAAAGUCUGAGUGGGCGGC.

In some embodiments, each mRNA encoding a distinct HSV antigen comprises a 3′ UTR comprising a distinct nucleotide sequence selected from SEQ ID NOs: 88-93. In some embodiments, the mRNA encoding HSV gB comprises a 3′ UTR comprising the nucleotide sequence of SEQ ID NO: 88. In some embodiments, the mRNA encoding HSV gC comprises a 3′ UTR comprising the nucleotide sequence of SEQ ID NO: 89. In some embodiments, the mRNA encoding HSV gD comprises a 3′ UTR comprising the nucleotide sequence of SEQ ID NO: 90. In some embodiments, the mRNA encoding HSV ICP0 comprises a 3′ UTR comprising the nucleotide sequence of SEQ ID NO: 91. In some embodiments, the mRNA encoding HSV ICP4 comprises a 3′ UTR comprising the nucleotide sequence of SEQ ID NO: 92.

In some embodiments, a 3′ UTR comprises, in 5′-to-3′ order: (a) the nucleic acid sequence UAAAGCUCCCCGGGGGCCUCGGUGGCCUAGCUUCUUGCCCCUUGGGCCUCCCCCC AGCCCCUCCUCCCCUUCCUGCAG (SEQ ID NO: 94), (b) an identification and ratio determination (IDR) sequence, and (c) the nucleic acid sequence UGGUCUUUGAAUAAAGUCUGAGUGGGCGGC (SEQ ID NO: 95). In some embodiments, each mRNA encoding a distinct HSV antigen comprises a 3′ UTR comprising, in 5′-to-3′ order: (a) the nucleotide sequence of SEQ ID NO: 94; (b) a distinct IDR sequence; and (c) the nucleotide sequence of SEQ ID NO: 95. IDR sequences are described herein in the section entitled “Identification and Ratio Determination (IDR) Sequences.” UTRs may also be omitted from the mRNA provided herein.

A 5′ UTR does not encode a protein (is non-coding). Natural 5′ UTRs have features that play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 96), where R is a purine (adenine or guanine) three bases upstream of the start codon (AUG), which is followed by another ‘G’0.5′UTR also have been known to form secondary structures which are involved in elongation factor binding.

In some embodiments of the disclosure, a 5′ UTR is a heterologous UTR, i.e., is a UTR found in nature associated with a different ORF. In other embodiments, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US2014/0206753, WO2013/185069), the sequence GGGAUCCUACC (SEQ ID NO: 97) (WO2014/144196) may also be used. In other embodiments, a 5′ UTR is a 5′ UTR of a TOP gene lacking the 5′ TOP motif (the oligopyrimidine tract) (e.g., WO2015/101414, WO2015/101415, WO2015/062738, WO2015/024667, WO2015/024667); 5′ UTR element derived from ribosomal protein Large 32 (L32) gene (WO/2015101414, WO2015101415, WO/2015/062738), 5′ UTR element derived from the 5′ UTR of an hydroxysteroid (17-0) dehydrogenase 4 gene (HSD17B4) (WO2015024667), or a 5′ UTR element derived from the 5′ UTR of ATP5A1 (WO2015/024667) can be used. In some embodiments, an internal ribosome entry site (IRES) is used instead of a 5′ UTR.

A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of mRNA of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hours, 12 hours, 1 day, 2 days, and 7 days post-transfection.

Those of ordinary skill in the art will understand that 5′ UTRs that are heterologous or synthetic may be used with any desired 3′ UTR sequence. For example, a heterologous or synthetic 5′ UTR may be used with a synthetic 3′ UTR or with a heterologous 3′ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US2010/0293625 and WO2015/085318A2, each of which is herein incorporated by reference.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type/native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US2010/0129877, which is incorporated herein by reference.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature, or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US 2009/0226470, herein incorporated by reference, and those known in the art.

### Open Reading Frames

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and/or 3′ UTRs, but that those elements, unlike the ORF, need not necessarily be present in an RNA (e.g., mRNA) of the present disclosure.

### 5′ End Capping

In some embodiments, an RNA (e.g., mRNA) comprises a 5′ terminal cap. 5′-capping of polynucleotides may be completed concomitantly during an in vitro transcription reaction using, for example, the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, MA). 5′-capping of modified RNA (e.g., mRNA) may be completed post-transcriptionally using, for example, a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source. Other cap analogs may be used.

### Polyadenylation Tailing

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. It can, in some instances, comprise up to about 400 adenine nucleotides. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation. In some embodiments, the length of the 3′-poly(A) tail may be an essential element with respect to the stability of the individual mRNA. In some embodiments, a poly(A) tail has a length of about 50, about 100, about 150, about 200, about 250, about 300, about 350, or about 400 nucleotides. In some embodiments, a poly(A) tail has a length of 100 nucleotides.

### Additional Stabilizing Elements

RNA (e.g., mRNA) provided herein, in some embodiments, includes an additional stabilizing element. Stabilizing elements may include, for example, a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, an RNA (e.g., mRNA) includes an open reading frame (coding region), a histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g., Luciferase, GFP, EGFP, β-Galactosidase, EGFP), or a marker or selection protein (e.g., alpha-Globin, Galactokinase and Xanthine:guanine phosphoribosyl transferase (GPT)).

In some embodiments, an RNA (e.g., mRNA) includes the combination of a poly(A) sequence or polyadenylation signal and at least one histone stem-loop, even though both represent alternative mechanisms in nature, they act synergistically to increase the protein expression beyond the level observed with either of the individual elements. The synergistic effect of the combination of poly(A) and a histone stem-loop does not depend on the order of the elements or the length of the poly(A) sequence.

In some embodiments, an RNA (e.g., mRNA) does not include a histone downstream element (HDE). “Histone downstream element” (HDE) includes a purine-rich polynucleotide stretch of approximately 15 to 20 nucleotides 3′ of naturally-occurring stem-loops, representing the binding site for the U7 snRNA, which is involved in processing of histone pre-mRNA into mature histone mRNA. In some embodiments, the nucleic acid does not include an intron.

An RNA (e.g., mRNA) may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, an RNA (e.g., mRNA) has one or more AU-rich sequences removed. These sequences, sometimes referred to as AURES are destabilizing sequences found in the 3′ UTR. The AURES may be removed from the mRNA. Alternatively, the AURES may remain in the mRNA.

### Sequence Optimization

In some embodiments, an open reading frame encoding a protein of the disclosure is codon optimized. Codon optimization methods are known in the art. An open reading frame of any one or more of the sequences provided herein may be codon optimized. Codon optimization, in some embodiments, may be used to match codon frequencies in target and host organisms to ensure proper folding; bias GC content to increase RNA (e.g., mRNA) stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and RNA (e.g., mRNA) degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame sequence is optimized using optimization algorithms.

In some embodiments, a codon optimized sequence shares less than 95% sequence identity to a naturally-occurring or wild-type sequence open reading frame (e.g., a naturally-occurring or wild-type RNA (e.g., mRNA) sequence encoding a HSV protein antigen). In some embodiments, a codon optimized sequence shares less than 90% sequence identity to a naturally-occurring or wild-type sequence (e.g., a naturally-occurring or wild-type RNA (e.g., mRNA) sequence encoding a HSV protein). In some embodiments, a codon optimized sequence shares less than 85% sequence identity to a naturally-occurring or wild-type sequence (e.g., a naturally-occurring or wild-type RNA (e.g., mRNA) sequence encoding a HSV protein). In some embodiments, a codon optimized sequence shares less than 80% sequence identity to a naturally-occurring or wild-type sequence (e.g., a naturally-occurring or wild-type RNA (e.g., mRNA) sequence encoding a HSV protein). In some embodiments, a codon optimized sequence shares less than 75% sequence identity to a naturally-occurring or wild-type sequence (e.g., a naturally-occurring or wild-type RNA (e.g., mRNA) sequence encoding a HSV protein).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type RNA (e.g., mRNA) sequence encoding a HSV protein). In some embodiments, a codon optimized sequence shares between 65% and 75% or about 80% sequence identity to a naturally-occurring or wild-type sequence (e.g., a naturally-occurring or wild-type RNA (e.g., mRNA) sequence encoding a HSV protein).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a HSV protein encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA (e.g., mRNA) may be one in which the levels of G/C are enhanced. The G/C-content of nucleic acid molecules (e.g., mRNA) may influence the stability of the RNA. RNA (e.g., mRNA) having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an RNA (e.g., mRNA) stabilized by sequence modifications in the translated region. Due to the degeneracy of the genetic code, the modifications work by substituting existing codons for those that promote greater RNA stability without changing the resulting amino acid. The approach is limited to coding regions of the RNA (e.g., mRNA).

### Chemically Unmodified Nucleotide

In some embodiments, an RNA (e.g., mRNA) is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g., A, G, C, or U). In some embodiments, nucleotides and nucleosides of the present disclosure comprise standard deoxyribonucleosides such as those present in DNA (e.g., dA, dG, dC, or dT).

### Chemically Modified Nucleotides

The compositions of the present disclosure comprise, in some embodiments, an RNA having an open reading frame encoding a HSV protein, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally-occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally-occurring modified nucleotides and nucleotides can be found, inter alia, in the widely recognized MODOMICS database.

In some embodiments, a non-naturally-occurring modified nucleotide or nucleoside of the disclosure is one as is generally known or recognized in the art. Non-limiting examples of such non-naturally-occurring modified nucleotides and nucleosides can be found, inter alia, in international publication numbers WO2013052523A1; WO2014093924A1; WO2015051173A2; WO2015051169A2; WO2015089511A2; or WO2017153936A1, each of which is herein incorporated by reference.

Hence, nucleic acids of the disclosure (e.g., DNA and RNA, such as mRNA) can comprise standard nucleotides and nucleosides, naturally-occurring nucleotides and nucleosides, non-naturally-occurring nucleotides and nucleosides, or any combination thereof.

Nucleic acids of the disclosure (e.g., DNA and RNA, such as mRNA), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA (e.g., mRNA) introduced to a cell or organism, exhibits reduced degradation in the cell or organism, respectively, relative to an unmodified nucleic acid comprising standard nucleotides and nucleosides.

In some embodiments, a modified RNA (e.g., mRNA) introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA, such as mRNA), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA, such as mRNA). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA, such as mRNA) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA, such as mRNA) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, an RNA (e.g., mRNA) of the disclosure comprises 1-methyl-pseudouridine (m1ψ) substitutions at one or more or all uridine positions of the nucleic acid.

In some embodiments, an RNA (e.g., mRNA) of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, an RNA (e.g., mRNA) of the disclosure comprises pseudouridine (ψ) substitutions at one or more or all uridine positions of the nucleic acid.

In some embodiments, an RNA (e.g., mRNA) of the disclosure comprises pseudouridine (ψ) substitutions at one or more or all uridine positions of the nucleic acid and 5-methyl cytidine substitutions at one or more or all cytidine positions of the nucleic acid.

In some embodiments, an RNA (e.g., mRNA) of the disclosure comprises uridine at one or more or all uridine positions of the nucleic acid.

In some embodiments, RNAs (e.g., mRNAs) are uniformly modified (e.g., fully modified, modified throughout the entire sequence) for a particular modification. For example, a nucleic acid can be uniformly modified with 1-methyl-pseudouridine, meaning that all uridine residues in the RNA (e.g., mRNA) sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the RNA (e.g., mRNA) including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The RNA (e.g., mRNA) may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure or can be replaced by a plurality of compounds having different structures (e.g., 2, 3, 4 or more unique structures).

### Nucleic Acid Production

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

The synthesis of nucleic acids of the present disclosure by the sequential addition of monomer building blocks may be carried out in a liquid phase.

The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### In Vitro Transcription

cDNA encoding the polynucleotides described herein may be transcribed using an in vitro transcription (IVT) system. In vitro transcription of RNA (e.g., mRNA) is known in the art and is described in International Publication WO 2014/152027, which is incorporated by reference herein in its entirety. In some embodiments, the RNA of the present disclosure is prepared in accordance with any one or more of the methods described in WO 2018/053209 or WO 2019/036682, each of which is incorporated by reference herein.

In some embodiments, the RNA (e.g., mRNA) transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of an RNA, for example, but not limited to HSV mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes an RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the RNA (e.g., mRNA) encoded by the template.

In some embodiments, a nucleic acid (e.g., template DNA and/or RNA) includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides.

An in vitro transcription system typically comprises a transcription buffer (e.g., with magnesium), nucleotide triphosphates (NTPs), an RNase inhibitor and a polymerase (e.g., T7 RNA polymerase). In some embodiments, one or more of the NTPs is a chemically modified NTP (e.g., with 1-methylpseudouridine or other chemical modifications described herein and/or known in the art).

In some embodiments, the NTPs comprise adenosine triphosphate (ATP), cytidine triphosphate (CTP), uridine triphosphate (UTP), and guanosine triphosphate (GTP), or an analog of each respective NTP. The ratio of NTPs may vary. In some embodiments, the ratio of GTP:ATP:CTP:UTP is 1:1:1:1. In some embodiments, the amount of the GTP or an analogue thereof is greater than an amount of the UTP or an analogue thereof. In some embodiments, the amount of the GTP is greater than the amount of the UTP. In some embodiments, the amount of ATP is greater than the amount of UTP, and the amount of CTP is greater than the amount of UTP. In some embodiments, the amount of the GTP or an analogue thereof is greater than an amount of the UTP or an analogue thereof. In some embodiments, an IVT system comprises an at least 2:1 ratio of GTP concentration to ATP concentration, an at least 2:1 ratio of GTP concentration to CTP concentration, and an at least 4:1 ratio of GTP concentration to UTP concentration. In some embodiments, an IVT system comprises a 2:1 ratio of GTP concentration to ATP concentration, a 2:1 ratio of GTP concentration to CTP concentration, and a 4:1 ratio of GTP concentration to UTP concentration. In some embodiments, an IVT system comprises guanosine diphosphate (GDP). In some embodiments, an IVT system comprises an at least 3:1 ratio of GTP plus GDP concentration to ATP concentration, an at least 6:1 ratio of GTP plus GDP concentration to CTP concentration, and an at least 6:1 ratio of GTP plus GDP concentration to UTP concentration.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

An IVT system, in some embodiments, comprises magnesium buffer, dithiothreitol (DTT) spermidine, pyrophosphatase, and/or RNase inhibitor. In some embodiments, an IVT system omits an RNase inhibitor. An IVT system may be incubated at 25 degrees Celsius or at 37 degrees Celsius. Other temperatures may be used, depending in part on the polymerase (e.g., use of a variant polymerase).

In some embodiments, the RNA transcript is capped via enzymatic capping. In some embodiments, the RNA comprises 5′ terminal cap, for example, 7mG(5′)ppp(5′)NlmpNp.

### Identification and Ratio Determination (IDR) Sequences

In some embodiments, one or more nucleic acids comprises an Identification and Ratio Determination sequence. An Identification and Ratio Determination (IDR) sequence is a sequence of a biological molecule (e.g., nucleic acid or protein) that, when combined with the sequence of a target biological molecule, serves to identify the target biological molecule. Typically, an IDR sequence is a heterologous sequence that is incorporated within or appended to a sequence of a target biological molecule and can be used as a reference to identify the target molecule. Thus, in some embodiments, a nucleic acid (e.g., mRNA) comprises (i) a target sequence of interest (e.g., a coding sequence encoding a therapeutic and/or antigenic peptide or protein); and (ii) a unique IDR sequence.

An RNA species (e.g., RNA having a given coding sequence) may comprise an IDR sequence that differs from the IDR sequence of other RNA species (e.g., RNA(s) having different coding sequence(s)). Each IDR sequence thus identifies a particular RNA species, and so the abundance of IDR sequences may be measured to determine the abundance of each RNA species in a composition. Use of distinct IDR sequences to identify RNA species allows for analysis of multivalent RNA compositions (e.g., containing multiple RNA species) containing RNA species with similar coding sequences and/or lengths, which could otherwise be difficult to distinguish using PCR— or chromatography-based analysis of full-length RNAs.

Each RNA species in a multivalent RNA composition may comprise an IDR sequence that is not a sequence isomer of an IDR sequence of another RNA species in a multivalent RNA composition (e.g., the IDR sequence does not have the same number of adenosine nucleotides, the same number of cytosine nucleotides, the same number of guanine nucleotides, and the same number of uracil nucleotides, as another IDR sequence in the composition, even if those sequences have different sequences). Having identical nucleotide compositions causes sequence isomers to have the same mass, presenting a challenge to distinguishing sequence isomers using mass-based identification methods (e.g., mass spectrometry).

Each RNA species in a multivalent RNA composition may comprise an IDR sequence having a mass that differs from the mass of IDR sequences of each other RNA species in a multivalent RNA composition. For example, the mass of each IDR sequence may differ from the mass of other IDR sequences by at least 9 Da, at least 25 Da, at least 25 Da, or at least 50 Da. Use of IDR sequences with distinct masses allows RNA fragments comprising different IDR sequences to be distinguished using mass-based analysis methods (e.g., mass spectrometry), which do not require reverse transcription, amplification, or sequencing of RNAs.

Each RNA species in an RNA composition may comprises an IDR sequence with a different length. For example, each IDR sequence may have a length independently selected from 0 to 25 nucleotides. The length of a nucleic acid influences the rate at which the nucleic acid traverses a chromatography column, and so the use of IDR sequences of different lengths on different RNA species allows RNA fragments having different IDR sequences to be distinguished using chromatography-based methods (e.g., LC-UV).

IDR sequences may be chosen such that no IDR sequence comprises a start codon, ‘AUG’. Lack of a start codon in an IDR sequence prevents undesired translation of nucleotide sequences within and/or downstream from the IDR sequence.

IDR sequences may be chosen such that no IDR sequence comprises a recognition site for a restriction enzyme. In one example, no IDR sequence comprises a recognition site for XbaI, ‘UCUAG’. Lack of a recognition site for a restriction enzyme (e.g., XbaI recognition site ‘UCUAG’) allows the restriction enzyme to be used in generating and modifying a DNA template for in vitro transcription, without affecting the IDR sequence or sequence of the transcribed RNA.

Non-limiting examples of distinct IDR sequences include: GAGAUUGAGUGUAGUGACUAG (SEQ ID NO: 98), GAGAUUGAGUGUAGUGAC (SEQ ID NO: 99), GAGAUUGAGUGUAGUG (SEQ ID NO: 100), GAUUGAGACUACGGG (SEQ ID NO: 101), and CAUAGACACUACG (SEQ ID NO: 102). In some embodiments of the compositions described herein, each mRNA encoding a distinct protein comprises a 3′ UTR comprising a distinct IDR sequence selected from SEQ ID NOs: 98-102.

### Lipid Compositions

In some embodiments, the nucleic acids are formulated as a lipid composition, such as a composition comprising a lipid nanoparticle, a liposome, and/or a lipoplex. In some embodiments, nucleic acids are formulated as lipid nanoparticle (LNP) compositions. Lipid nanoparticles typically comprise amino lipid, non-cationic lipid, structural lipid, and PEG lipid components along with the nucleic acid cargo of interest. The lipid nanoparticles can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2017/038426; PCT/US2014/027077; PCT/US2014/055394; PCT/US2016/052117; PCT/US2012/069610; PCT/US2017/027492; PCT/US2016/059575; PCT/US2016/069491; PCT/US2016/069493; and PCT/US2014/066242, all of which are incorporated by reference herein in their entirety.

In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid, 5-25% non-cationic lipid, 25-55% structural lipid, and 0.5-15% PEG-modified lipid.

In some embodiments, the lipid nanoparticle comprises a molar ratio of 20-60% ionizable amino lipid, 5-30% non-cationic lipid, 10-55% structural lipid, and 0.5-15% PEG-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

### Ionizable Amino Lipids

### Formula (AI)

In some embodiments, the ionizable amino lipid of the present disclosure is a compound of Formula (A1):

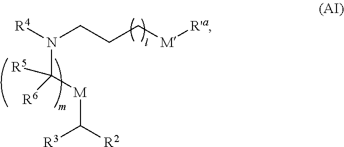

or its N-oxide, or a salt or isomer thereof,
wherein R′^{a} is R′^{branched}; wherein R′^{branched}

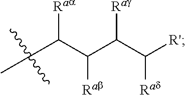

wherein [symbol] denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

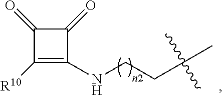

wherein [symbol] denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (AI), R′^{a} is R′^{branched}; R′^{branched} is

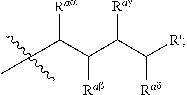

[symbol]

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}; R′^{branched} is

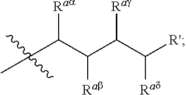

[symbol]

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (AI), R′^{a} is R′^{branched}; R′^{branched} is

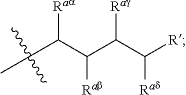

[symbol]

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is H

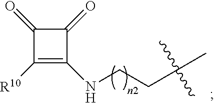

R¹⁰ NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}; R′^{branched} is

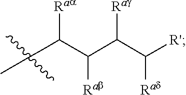

[symbol]

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the compound of Formula (AI) is selected from:

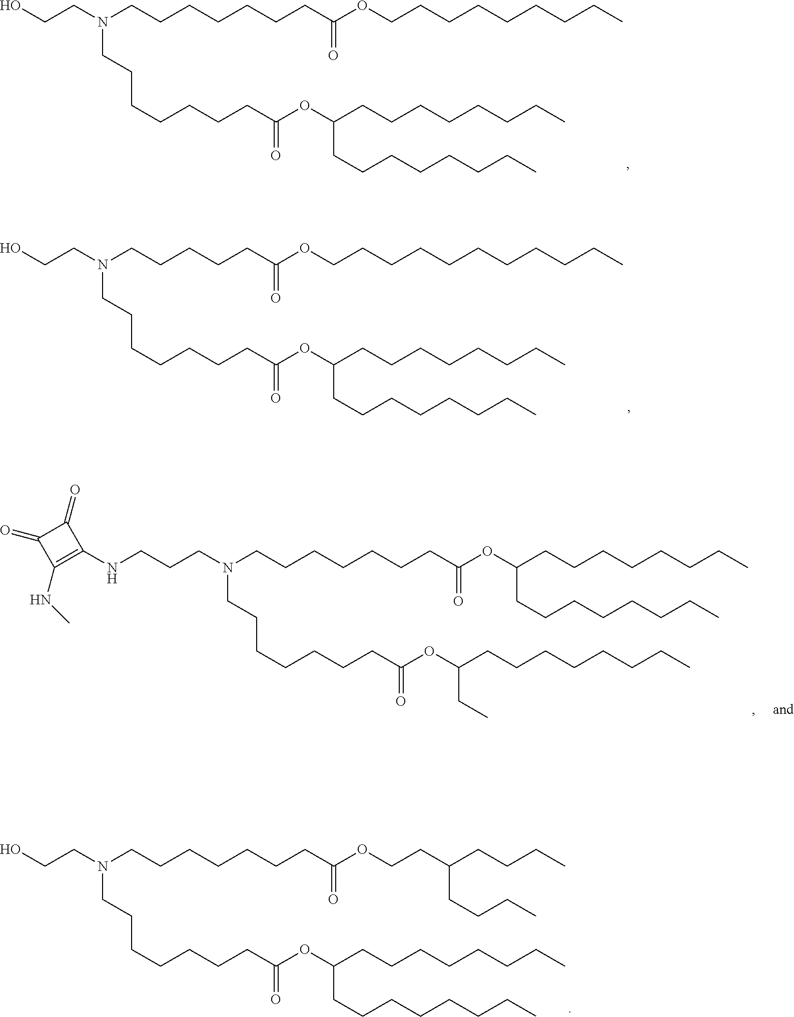 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the ionizable amino lipid of Formula (AI) is a compound of Formula (AIa):

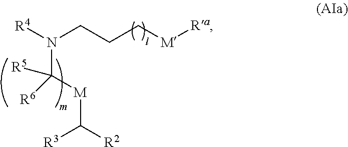

or its N-oxide, or a salt or isomer thereof,
wherein R′^{a} is R′^{branched}; wherein
R′^{branched} is:

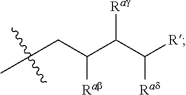

wherein [symbol] denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

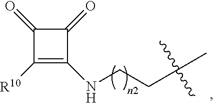

wherein [symbol] denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid of Formula (AI) is a compound of Formula (AIb):

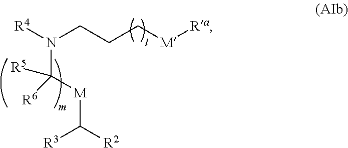

or its N-oxide, or a salt or isomer thereof,
wherein R′^{a} is R′^{branched}; wherein
R′^{branched} is:

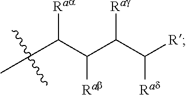

wherein [symbol] denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (AI) or (AIb), R′^{a} is R′^{branched}; R′^{branched} is

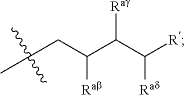

[symbol]

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of Formula (AI) or (AIb), R′^{a} is R′^{branched}; R′^{branched} is

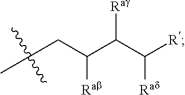

[symbol]

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of Formula (AI) or (AIb), R′^{a} is R′^{branched}; R′^{branched} is

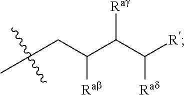

[symbol]

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the ionizable amino lipid of Formula (AI) is a compound of Formula (AIc):

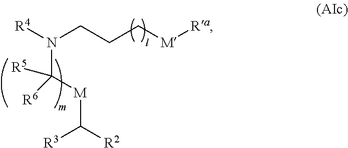

or its N-oxide, or a salt or isomer thereof,
wherein R′^{a} is R′^{branched}; wherein
R′^{branched} is:

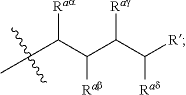

wherein [symbol] denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

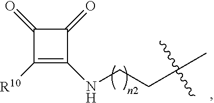

wherein [symbol] denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

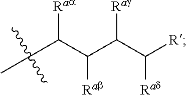

[symbol]

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

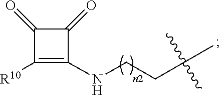

[symbol]

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; 1 is 5; and m is 7.

In some embodiments, the compound of Formula (AIc) is:

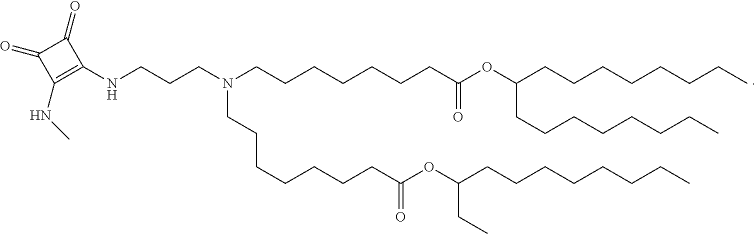 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

### Formula (AII)

In some embodiments, the ionizable amino lipid is a compound of Formula (AII):

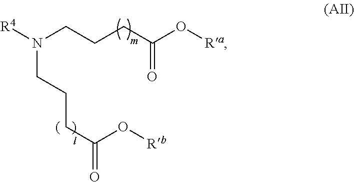

or its N-oxide, or a salt or isomer thereof,
wherein R′^{a} is R′^{branched} or R′^{cyclic}; wherein
R′^{branched} is:

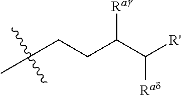

### and R′^{cyclic} is:

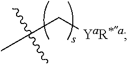

and
R′^{b} is:

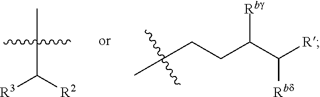

wherein [symbol] denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

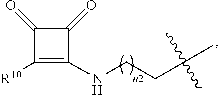

wherein [symbol] denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-a):

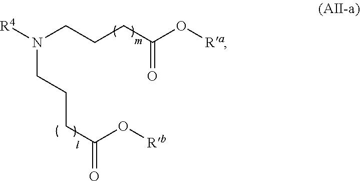

or its N-oxide, or a salt or isomer thereof,
wherein R′^{a} is R′^{branched} or R′^{cyclic}; wherein
R′^{branched} is:

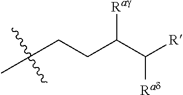

### and R′^{b} is:

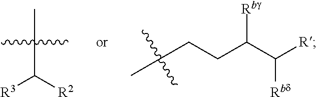

wherein [symbol] denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

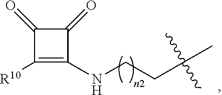

wherein [symbol] denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-b):

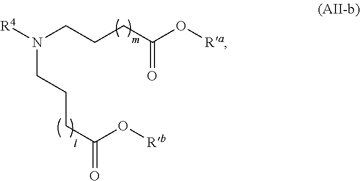

or its N-oxide, or a salt or isomer thereof,
wherein R′^{a} is R′^{branched} or R′^{cyclic}; wherein
R′^{branched} is:

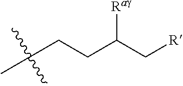

### and R′^{b} is:

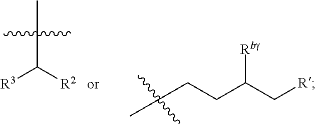

wherein [symbol] denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

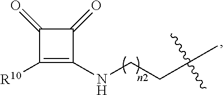

wherein [symbol] denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-c):

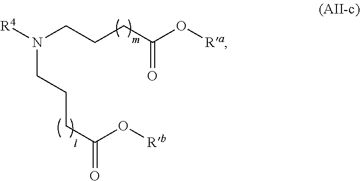

or its N-oxide, or a salt or isomer thereof,
wherein R′^{a} is R′^{branched} or R′^{cyclic}; wherein
R′^{branched} is:

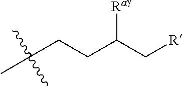

### and R′^{b} is:

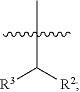

wherein [symbol] denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

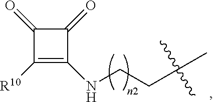

wherein [symbol] denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-d):

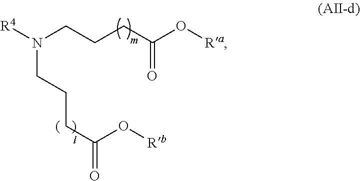

or its N-oxide, or a salt or isomer thereof,
wherein R′^{a} is R′^{branched} or R′^{cyclic}; wherein
R′^{branched} is:

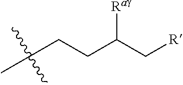

### and R′ is:

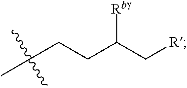

wherein [symbol] denotes a point of attachment;
wherein R^{aγ} and R^{aγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

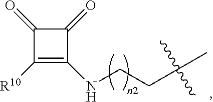

wherein [symbol] denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-e):

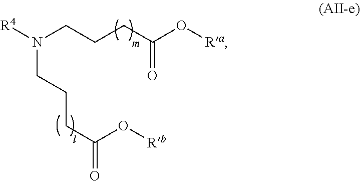

or its N-oxide, or a salt or isomer thereof,
wherein R′ a is R′ branched or R′^{cyclic}; wherein
R′^{branched} is:

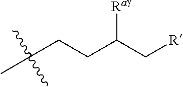

### and R′^{b} is:

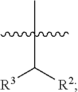

wherein [symbol] denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (AII), (AII-a), (AII-b), (AII-c), (AII-d), or (AII-e), m and l are each independently selected from 4, 5, and 6. In some embodiments of the compound of Formula (AII), (AII-a), (AII-b), (AII-c), (AII-d), or (AII-e), m and l are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C2-s alkyl.

In some embodiments of the compound of Formula (AII), (AII-a), (AII-b), (AII-c), (AII-d), or (AII-e), R′^{b} is:

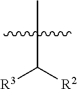

and R² and R³ are each independently a C₁₋₁₄ alkyl. In some embodiments of the compound of Formula (AII), (AII-a), (AII-b), (AII-c), (AII-d), or (AII-e), R′^{b} is:

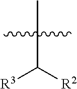

and R² and R³ are each independently a C₆₋₁₀ alkyl. In some embodiments of the compound of Formula (AII), (AII-a), (AII-b), (AII-c), (AII-d), or (AII-e), R′^{b} is:

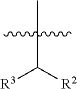

and R² and R³ are each a C₈ alkyl.

In some embodiments of the compound of Formula (AII), (AII-a), (AII-b), (AII-c), (AII-d), or (AII-e), R′^{branched} is:

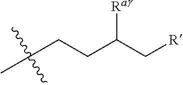

### and R′^{b} is:

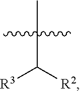

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

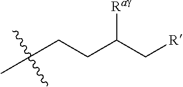

### and R′^{b} is:

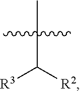

R^{aγ} is a C₂₋₆ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

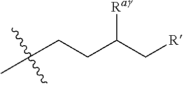

### and R′^{b} is:

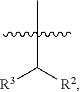

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

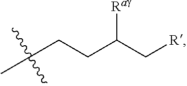

### R′^{b} is:

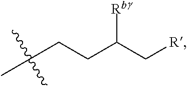

and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

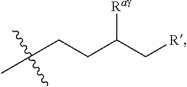

### R′^{b} is:

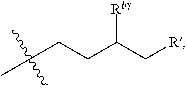

and R^{aγ} and R^{bγ} are each a C₂₋₆ alkyl.

In some embodiments of the compound of Formula (AII), (AII-a), (AII-b), (AII-c), (AII-d), or (AII-e), m and l are each independently selected from 4, 5, and 6 and each R′ independently is a C₁₋₁₂ alkyl. In some embodiments of the compound of Formula (AII), (AII-a), (AII-b), (AII-c), (AII-d), or (AII-e), m and l are each 5 and each R′ independently is a C2-s alkyl.

In some embodiments of the compound of (AII), (AII-a), (AII-b), (AII-c), (AII-d), or (AII-e), R′^{branched} is:

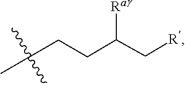

### R′^{b} is:

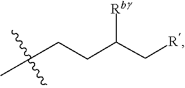

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

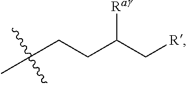

### R′^{b} is:

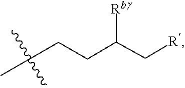

m and l are each 5, each R′ independently is a C₂₋₅ alkyl, and R^{aγ} and R^{b} are each a C₂₋₆ alkyl.

In some embodiments of the compound of Formula (AII), (AII-a), (AII-b), (AII-c), (AII-d), or (AII-e), R′^{branched} is:

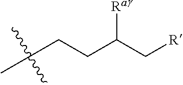

### and R′^{b} is:

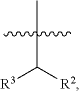

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

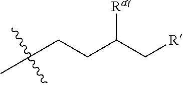

### and R′^{b} is:

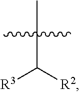

m and l are each 5, R′ is a C₂₋₅ alkyl, R^{aγ} is a C₂₋₆ alkyl, and R² and R³ are each a C₈ alkyl.

In some embodiments of the compound of (AII), (AII-a), (AII-b), (AII-c), (AII-d), or (AII-e), R⁴ is

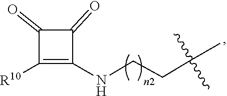

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

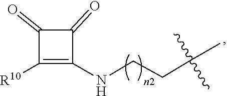

wherein R¹⁰ is NH(CH₃) and n2 is 2.

In some embodiments of the compound of Formula (AII), (AII-a), (AII-b), (AII-c), (AII-d), or (AII-e), R′^{branched} is:

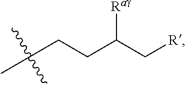

### R′^{b} is:

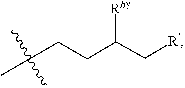

m and l are each independently selected from 4, 5, and 6, each R′ independently is a C₁₋₁₂ alkyl, R^{aγ} and R^{aγ} are each a C₁₋₁₂ alkyl, and R⁴ is

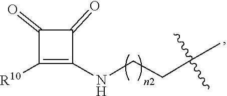

wherein R¹⁰ is NH(C₁₋₆ alkyl), and n2 is 2. In some embodiments of the compound of Formula (AII), (AII-a), (AII-b), (AII-c), (AII-d), or (AII-e), R′^{branched} is:

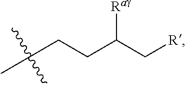

### R′^{b} is:

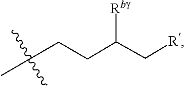

m and l are each 5, each R′ independently is a C₂₋₅ alkyl, R^{aγ} and R^{bγ} are each a C₂₋₆ alkyl, and R⁴ is

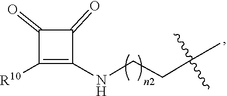

wherein R¹⁰ is NH(CH₃) and n2 is 2.

In some embodiments of the compound of Formula (AII), (AII-a), (AII-b), (AII-c), (AII-d), or (AII-e), R′^{branched} is:

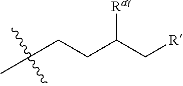

### and R′^{b} is:

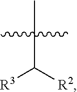

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

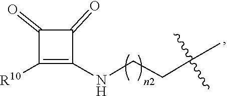

wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is:

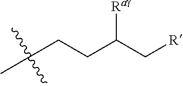

### and R′^{b} is:

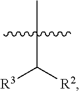

m and l are each 5, R′ is a C₂₋₅ alkyl, R^{aγ} is a C₂₋₆ alkyl, R² and R³ are each a C₈ alkyl, and R⁴ is

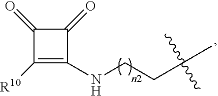

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂)ₙOH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

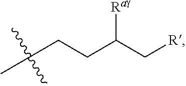

### R′^{b} is:

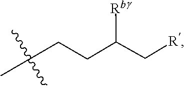

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AII), (AII-a), (AII-b), (AII-c), (AII-d), or (AII-e), R′^{branched} is:

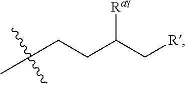

### R′^{b} is:

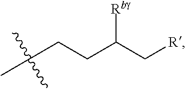

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid of Formula (AII) is a compound of Formula (AII-f):

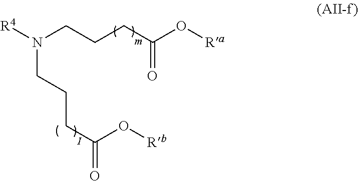

or its N-oxide, or a salt or isomer thereof,
wherein R′^{a} is R′^{branched} or R′^{cyclic}; wherein
R′^{branched} is:

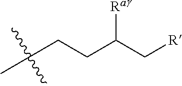

### and R′^{b} is:

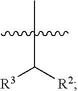

wherein [symbol] denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (AII-f), m and l are each 5, and n is 2, 3, or 4.

In some embodiments of the compound of Formula (AII-f) R′ is a C₂₋₅ alkyl, R^{aγ} is a C₂₋₆ alkyl, and R² and R³ are each a C₆₋₁₀ alkyl.

In some embodiments of the compound of Formula (AII-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the ionizable amino lipid of Formula (AII) is a compound of Formula (AII-g):

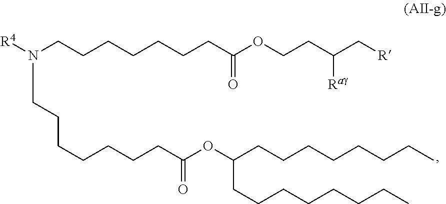

or its N-oxide, or a salt or isomer thereof; wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

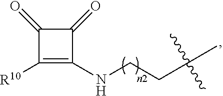

wherein [symbol] denotes a point of attachment, R¹⁰ is NH(C₁₋₆ alkyl), and n2 is selected from the group consisting of 1, 2, and 3.

In some embodiments, the ionizable amino lipid of Formula (AII) is a compound of Formula (AII-h):

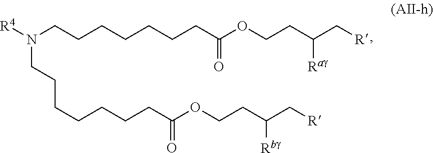

or its N-oxide, or a salt or isomer thereof; wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

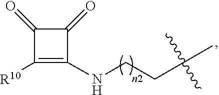

wherein [symbol] denotes a point of attachment, R¹⁰ is NH(C₁₋₆ alkyl), and n2 is selected from the group consisting of 1, 2, and 3.

In some embodiments of the compound of Formula (AII-g) or (AII-h), R⁴ is

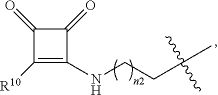

wherein
R¹⁰ is NH(CH₃) and n2 is 2.

In some embodiments of the compound of Formula (AII-g) or (AII-h), R⁴ is —(CH₂)₂OH.

### Formula (AII)

In some embodiments, the ionizable amino lipids of the present disclosure may be one or more of compounds of Formula (AIII):

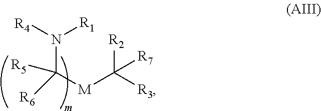

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which:
R₁ is selected from the group consisting of C₅-30 alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In certain embodiments, a subset of compounds of Formula (AIII) includes those of Formula (AIII-A):

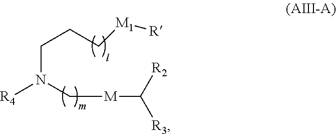

or its N-oxide, or a salt or isomer thereof, wherein 1 is selected from 1, 2, 3, 4, and 5; m is selected from 5, 6, 7, 8, and 9; M1 is a bond or M′; R₄ is hydrogen, unsubstituted C₁₋₃ alkyl, or —(CH₂)ₙQ, in which Q is —OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group, and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (AIII) includes those of Formula (AIII-B):

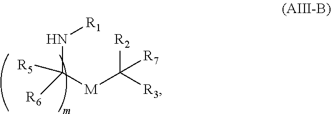

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is H, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (AIII) includes those of Formula (AIII-C):

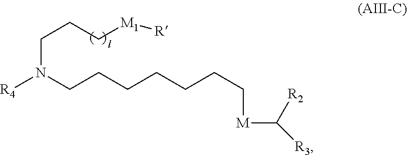

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, the compounds of Formula (AIII) are of Formula (AIII-D),

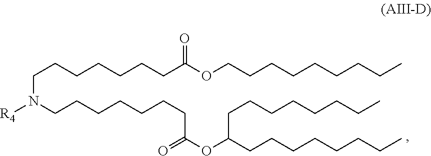

or their N-oxides, or salts or isomers thereof, wherein R₄ is as described herein.

In another embodiment, the compounds of Formula (AIII) are of Formula (AIII-E),

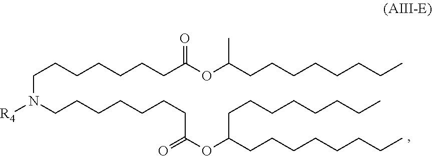

or their N-oxides, or salts or isomers thereof, wherein R₄ is as described herein.

In another embodiment, the compounds of Formula (AIII) are of Formula (AIII-F) or (AIII-G):

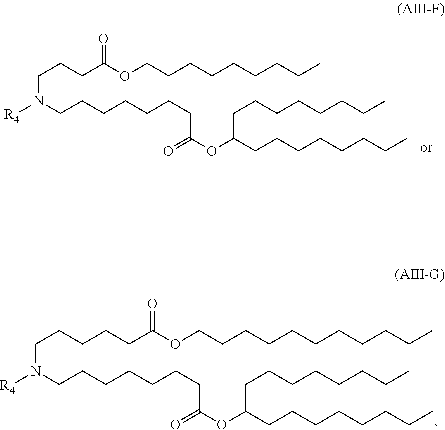

or their N-oxides, or salts or isomers thereof, wherein R₄ is as described herein.

In another embodiment, the compounds of Formula (AIII) are of Formula (AIII-H):

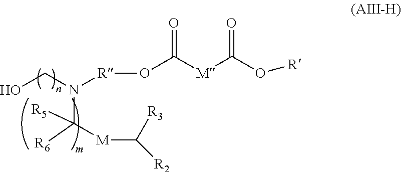

or their N-oxides, or salts or isomers thereof,
wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (AIII) are of Formula (AIII-I):

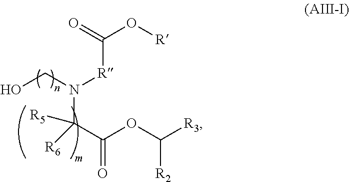

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

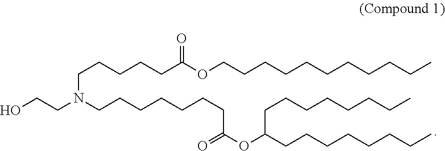 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

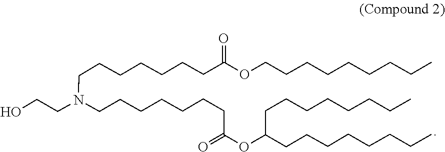 [SMILES: CCCCCCCCCOC(=O)CCCCCCCN(CCO)CCCCCCCC(=O)OC(CCCCCCCC)CCCCCCCC]

In a further embodiment, the compounds of Formula (AIII) are of Formula (AIII-J),

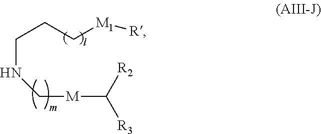

or their N-oxides, or salts or isomers thereof, wherein 1 is selected from 1, 2, 3, 4, and 5; m is selected from 5, 6, 7, 8, and 9; M1 is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable amino lipids are of Formula (AIII), or salts or isomers thereof, wherein:
R¹ is —R″M′R′;
R₂ and R₃ are each independently selected from C₁₋₁₄ alkyl and C₂₋₁₄ alkenyl;
R₄ is —(CH₂)ₙQ, wherein Q is OH and n is selected from 3, 4, and 5;
M and M′ are each independently —OC(O)—;
R₅, R₆, and R₇ are each H;
R′ is a linear C₁₋₁₂ alkyl, or C₁₋₁₂ alkyl substituted with C₆₋₉ alkyl;
R″ is C₃₋₁₄ alkyl;
m is selected from 5, 6, 7, 8, 9, 10, 11, 12, and 13.

In some embodiments, the ionizable amino lipids are of Formula (AIII), or salts or isomers thereof, wherein:
R¹ is R″M′R′;
R₂ and R₃ are each independently C₁₋₁₄ alkyl;
R₄ is —(CH₂)ₙQ, wherein Q is OH and n is 4;
M and M′ are each independently —OC(O)—;
R₅, R₆, and R₇ are each H;
R′ is C₁₋₁₂ alkyl substituted with C₆₋₉ alkyl;
R″ is C₃₋₁₄ alkyl; and
m is 6.

In some embodiments, an ionizable amino lipid of the disclosure comprises a compound having structure:

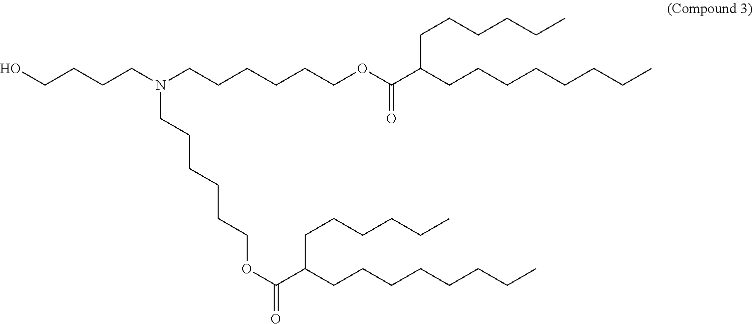 [SMILES: CCCCCCCCC(CCCCCC)C(=O)OCCCCCCN(CCCCO)CCCCCCOC(=O)C(CCCCCC)CCCCCCCC]

In some embodiments, the ionizable amino lipids are of Formula (AIII), or salts or isomers thereof, wherein:
R¹ is C₅₋₂₀ alkenyl;
R₂ and R₃ are each independently selected from C₁₋₁₄ alkyl and C₂₋₁₄ alkenyl;
R₄ is —(CH₂)ₙQ, wherein Q is OH and n is selected from 3, 4, and 5;
M and M′ are each independently C(O)O—;
R₅, R₆, and R₇ are each H;
R′ is a linear C₁₋₁₂ alkyl, or C₁₋₁₂ alkyl substituted with C₆₋₉ alkyl;
R″ is C₃₋₁₄ alkyl;
m is selected from 5, 6, 7, 8, 9, 10, 11, 12, and 13.

In some embodiments, the ionizable amino lipids are of Formula (AIII), or salts or isomers thereof, wherein:
R₁ is C₅₋₂₀ alkenyl;
R₂ and R₃ are each independently C₁₋₁₄ alkyl;
R₄ is —(CH₂)ₙQ, wherein Q is OH and n is 3;
M is —C(O)O—;
R₅, R₆, and R₇ are each H; and
m is 6.

In some embodiments, an ionizable amino lipid of the disclosure comprises a compound having structure:

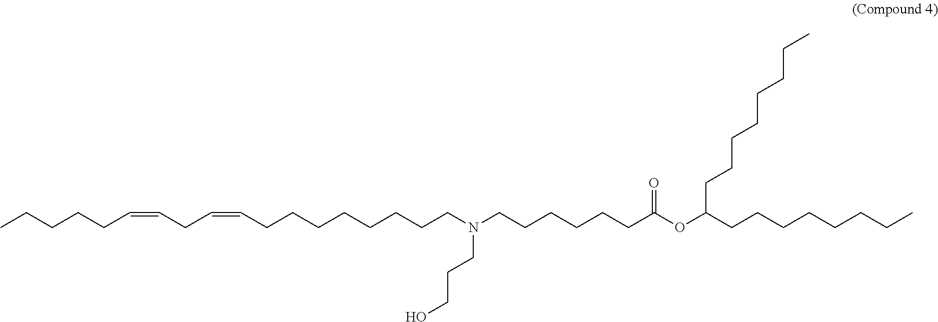 [SMILES: CCCCC/C=C\C/C=C\CCCCCCCCN(CCCO)CCCCCCC(=O)OC(CCCCCCCC)CCCCCCCC]

In some embodiments, the ionizable amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

The central amine moiety of a lipid according to Formula (AIII), (AIII-A), (AIII-B), (AIII-C), (AIII-D), (AIII-E), (AIII-F), (AIII-G), (AIII-H), (AIII-I), or (AIII-J) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

### Formula (AIV)

In some embodiments, the ionizable amino lipids of the present disclosure may be one or more of compounds of formula (AIV),

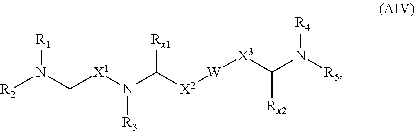

or salts or isomers thereof, wherein
W is

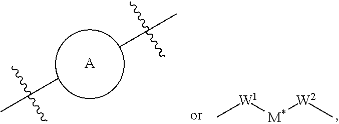

ring A is

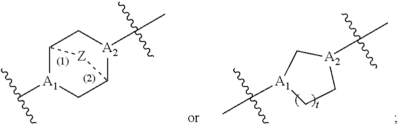

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
Rₓ₁ and Rₓ₂ are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R⁶)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

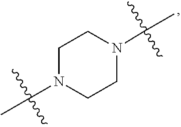 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (AIVa)-(AIVh):

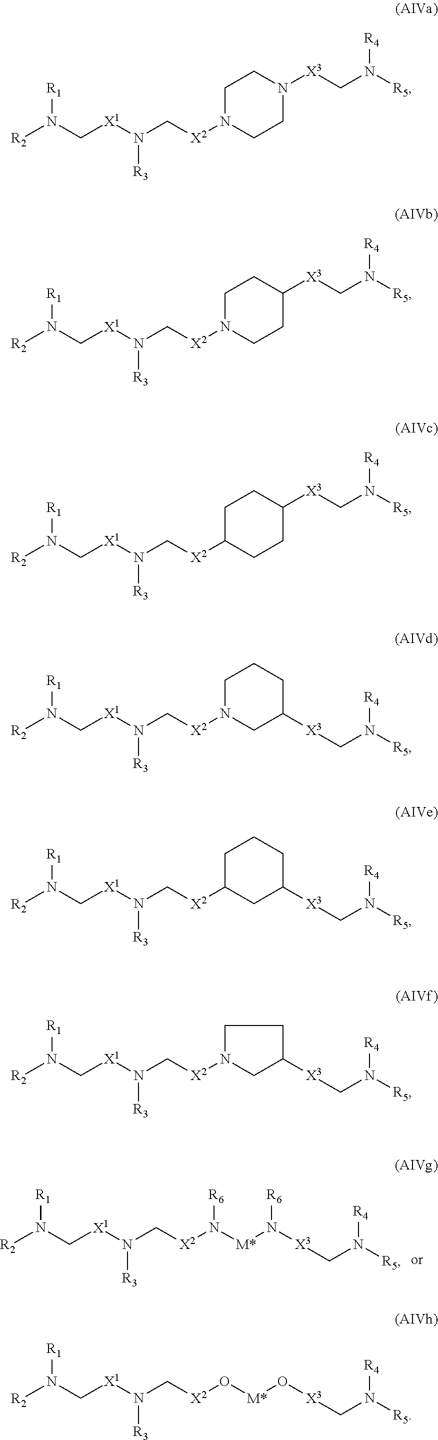

In some embodiments, the ionizable amino lipid is

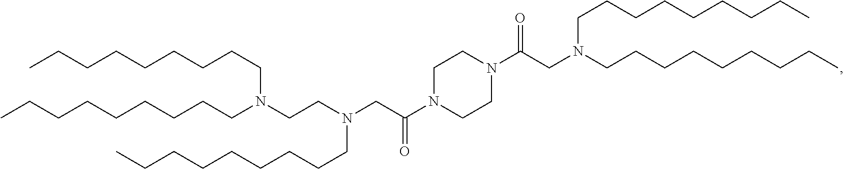 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (AIV), (AIVa), (AIVb), (AIVc), (AIVd), (AIVe), (AIVf), (AIVg), or (AIVh) may be protonated at a physiological pH. Thus, a lipid may have a positive or partial positive charge at physiological pH.

### Formula (AV)

In some embodiments, the lipid nanoparticle comprises a lipid having the structure:

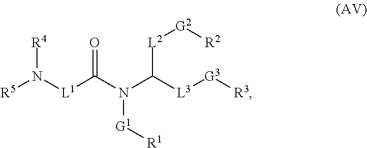

or a pharmaceutically acceptable salt, tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C₁-C₁₈ alkylene;
G¹ is a direct bond, —(CH₂)ₙO(C═O)—, —(CH₂)ₙ(C═O)O—, or —(C═O)—;
G² and G³ are each independently —(C═O)O— or —O(C═O)—; and n is an integer greater than 0.

### Formula (AVI)

In some embodiments, the lipid nanoparticle comprises a lipid having the structure:

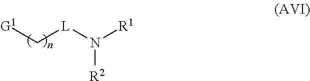

or a pharmaceutically acceptable salt, tautomer, or stereoisomer thereof, wherein:
G¹ is —N(R³)R⁴ or —OR⁵;
R¹ is optionally substituted branched, saturated or unsaturated C₁₂-C₃₆ alkyl;
R² is optionally substituted branched or unbranched, saturated or unsaturated C₁₂-C₃₆alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and
n is an integer from 1 to 12.

### Formula (AVII)

In some embodiments, the lipid nanoparticle comprises a lipid having the structure:

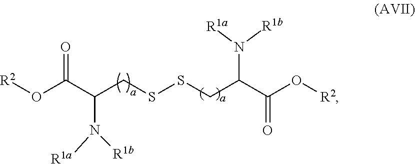

or a pharmaceutically acceptable salt thereof, wherein:
each R^{1a} is independently hydrogen, R^{1c}, or R^{1d};
each R^{1b} is independently R^{1c} or R^{1d};
each R^{1c} is independently —[CH₂]₂C(O)X¹R³;
each R^{1d} Is independently —C(O)R⁴;
each R² is independently —[C(R^{2a})₂]_{c}R^{2b};
each R^{2a} is independently hydrogen or C₁-C₆ alkyl;
R^{2b} is —N(L₁-B)₂; —(OCH₂CH₂)₆OH; or —(OCH₂CH₂)_{b}OCH₃;
each R³ and R⁴ is independently C₆-C₃₀ aliphatic;
each I.₃ is independently C₁-C₁₀ alkylene;
each B is independently hydrogen or an ionizable nitrogen-containing group;
each X¹ is independently a covalent bond or O;
each a is independently an integer of 1-10;
each b is independently an integer of 1-10; and
each c is independently an integer of 1-10.

### Formula (AVIII)

In some embodiments, the lipid nanoparticle comprises a lipid having the structure:

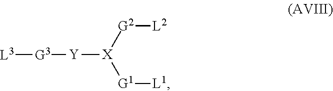

or a pharmaceutically acceptable salt, prodrug or stereoisomer thereof, wherein:
X is N, and Y is absent; or X is CR, and Y is NR;
L¹ is —O(C—O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R².
L³ is —O(C═O)R³ or —(C═O)OR³;
G¹ and G² are each independently C₂-C₁₂ alkylene or C₂-C₁₂ alkenylene;
G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
each R is independently H or C₁-C₁₂ alkyl;
R¹, R² and R³ are each independently C₁-C₂₄ alkyl or C₂-C₂₄ alkenyl; and x is 0, 1 or 2, and
wherein each alkyl, alkenyl, alkylene, alkenylene, heteroalkylene and heteroalkenylene is independently substituted or unsubstituted unless otherwise specified.

### Formula (AIX)

In some embodiments, the lipid nanoparticle comprises a lipid having the structure:

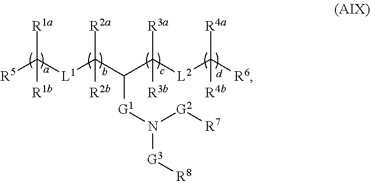

or a pharmaceutically acceptable salt, tautomer, prodrug or stereoisomer thereof, wherein:
L¹ and L² are each independently —O(C═O)—, —(C═O)O—, —C(═O)—, —O—, —S(O)x-ₛ —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C,—C₂ alkylene, —(C═O)—, —O(C═O)—, —SC(═O)—, —NR^{a}C(═O)— or a direct bond;
G² is —C(O)—, —(CO)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b)
R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4A} and R^{4B} are, at each occurrence, independently either: (a) H or C₁-C₁₂ alkyl; or (b) R^{4A} is H or C₁-C₁₂ alkyl, and R^{4B} together with the carbon atom to which it is bound is taken together with an adjacent R^{4B} and the carbon atom to which it is bound to form a carbon-carbon double bond;
R⁵ and R⁶ are each independently H or methyl;
R⁷ is H or C,—C₂₀ alkyl;
R₈ is OH, —N(R⁹)(C═O)R¹⁰, —(C═O)NR⁹R¹⁰, —NR⁹R¹⁰, —(C═O)OR″¹ or —O(C═O)R″,
provided that G³ is C₄-C₆ alkylene when R₈ is —NR⁹R¹⁰,
R⁹ and R¹⁰ are each independently H or C₁-C₁₂ alkyl;
R″ is aralkyl;
a, b, c and d are each independently an integer from 1 to 24; and x is 0, 1 or 2,
wherein each alkyl, alkylene and aralkyl is optionally substituted.

### Formula (AX)

In some embodiments, the lipid nanoparticle comprises a lipid having the structure:

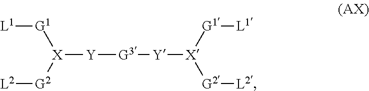

or a pharmaceutically acceptable salt, prodrug or stereoisomer thereof, wherein:
X and X′ are each independently N or CR;
Y and Y′ are each independently absent, —O(C═O)—, —(C═O)O— or NR, provided that:
   a) Y is absent when X is N;
   b) Y′ is absent when X′ is N;
   c) Y is —O(C═O)—, —(C═O)O— or NR when X is CR; and
   d) Y′ is —O(C═O)—, —(C═O)O— or NR when X′ is CR,
L¹ and L^{1′} are each independently —O(C═O)R′, —(C═O)OR′, —C(═O)R′, —OR¹, —S(O)_{z}R′, —S—SR¹, —C(═O)SR′, —SC(═O)R′, —NR^{a}C(═O)R′, —C(═O)NR^{b}R^{c}, —NR^{a}C(═O)NR^{b}R^{c}, —OC(═O)NR^{b}R′ or —NR^{a}C(═O)OR′;
L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R².
G¹, G^{1′}, G² and G^{2′} are each independently C₂-C₁₂ alkylene or C₂-C₁₂ alkenylene;
G is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{c} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and wherein each alkyl, alkenyl, alkylene, alkenylene, heteroalkylene and heteroalkenylene is independently substituted or unsubstituted unless otherwise specified.

### Formula (AXI)

In some embodiments, the lipid nanoparticle comprises a lipid having the structure:

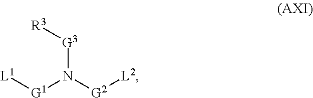

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R².
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl; and
x is 0, 1 or 2, and
wherein each alkyl, alkenyl, alkylene, alkenylene, cycloalkylene, cycloalkenylene, aryl and aralkyl is independently substituted or unsubstituted unless otherwise specified.

In some embodiments, the lipid nanoparticle comprises a lipid having the structure:

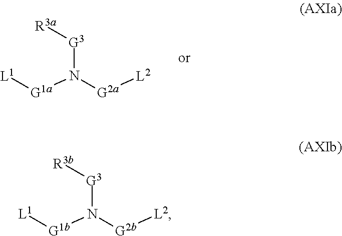

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R².
G^{1a} and G^{2b} are each independently C₂-C₁₂ alkylene or C₂-C₁₂ alkenylene;
G¹b and G^{2b} are each independently C₁-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R^{3a} is —C(═O)N(R^{4a})R^{5a} or —C(═O)OR⁶;
R^{3b} is —NR^{4b}C(═O)R^{5b};
R^{4a} is C₁-C₁₂ alkyl;
R^{4b} is H, C₁-C₁₂ alkyl or C₂-C₁₂ alkenyl;
R^{5a} is H, C₁-C₈alkyl or C₂-C₅ alkenyl;
R^{5b} is C₂-C₁₂ alkyl or C₂-C₁₂ alkenyl when R^{4b} is H; or R^{5b} is C₁-C₁₂ alkyl or C₂-C₁₂ alkenyl when R^{4b} is C₁-C₁₂ alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

### Formula (AXII)

In some embodiments, the lipid nanoparticle comprises a lipid having the structure:

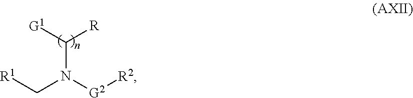

or a pharmaceutically acceptable salt, prodrug or stereoisomer thereof, wherein:
G¹ is —OH, —R³R⁴, —(C═O) R⁵ or —R³(C═O)R⁵;
G² is —CH₂— or —(C═O)—;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated C₁-C₆ alkyl;
R⁵ is optionally substituted straight or branched, saturated or unsaturated C₁-C₆ alkyl; and
n is an integer from 2 to 6.

### Formula (AXIII)

In some embodiments, the lipid nanoparticle comprises a lipid having the structure:

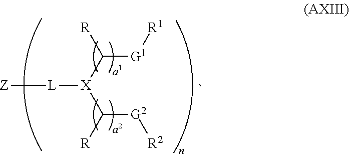

or a pharmaceutically acceptable salt, prodrug or stereoisomer thereof, wherein:
one of G¹ or G² is, at each occurrence, —O(C═O)—, —(C═O)O—, —C(═O)—, —O—, —S(O), —S—S—, —C(═O)S—, SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;
L is, at each occurrence, ˜O(C═O)—, wherein˜represents a covalent bond to X;
X is CR^{a};
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

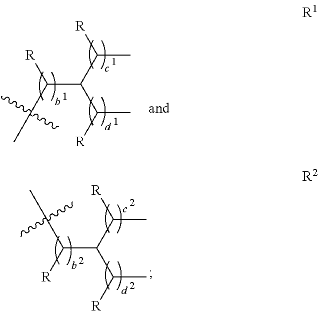

a¹ and a² are, at each occurrence, independently an integer from 3 to 12; b¹ and b² are, at each occurrence, independently 0 or 1;
c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

### Formula (AXIV)

In some embodiments, the lipid nanoparticle comprises a lipid having the structure:

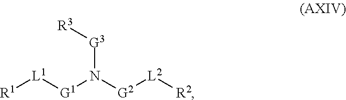

or a pharmaceutically acceptable salt, prodrug or stereoisomer thereof, wherein:
one of L¹ or L² is —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, R^{a}C(═O)R^{a}—, —OC(═O)R^{a}— or —R^{a}C(═O)O—, and the other of L¹ or L² is —O(C═O)—, —(C═O)O—, —C(═O)—, —O—, —S(O)X—, —S—S—, —C(═O)S—, SC(═O)—, —R^{a}C(═O)—, —C(═O)R^{a}—, R^{a}C(═O)R^{a}—, —OC(═O)R^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —R⁵C(═O)R⁴;
R⁴ is C₁-C₁₂ alkyl;
R⁵ is H or C₁-C₆ alkyl; and
x is 0, 1 or 2.

### Formula (AXV)

In some embodiments, the lipid nanoparticle comprises a lipid having the structure:

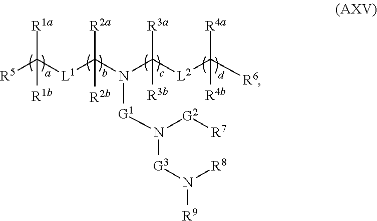

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, —R^{a}C(═O)R^{a}—, —OC(═O)R^{a}—, —R^{a}C(═O)O— or a direct bond;
G¹ is Ci-C₂ alkylene, —(C═O)—, —O(C═O)—, —SC(═O)—, —R^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

### Formula (AXVI)

In some embodiments, the lipid nanoparticle comprises a lipid having the structure:

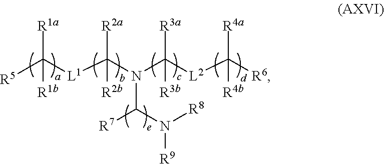

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24; b and c are each independently an integer from 1 to 24; and e is 1 or 2,
provided that:
at least one of R¹, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

### Formula (AXVII)

In some embodiments, the lipid nanoparticle comprises a lipid having the structure:

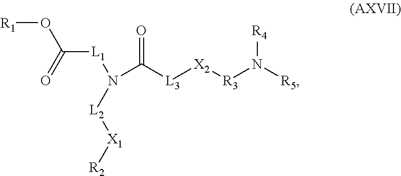

or a pharmaceutically acceptable salt thereof, wherein
R₁ and R₂ are the same or different, each a linear or branched alkyl with 1-9 carbons, or as alkenyl or alkynyl with 2 to 11 carbon atoms,
L₁ and L₂ are the same or different, each a linear alkyl having 5 to 18 carbon atoms, or form a heterocycle with N,
X₁ is a bond, or is —CG-G- whereby L₂-CO—O—R₂ is formed,
X₂ is S or O,
L₃ is a bond or a lower alkyl, or form a heterocycle with N,
R₃ is a lower alkyl, and
R₄ and R₅ are the same or different, each a lower alkyl.

### Compounds (A1)-(A11)

In some embodiments, the lipid nanoparticle comprises an ionizable lipid having the structure:

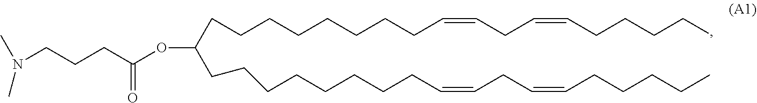

or a pharmaceutically acceptable salt thereof.

In some embodiments, the lipid nanoparticle comprises a lipid having the structure:

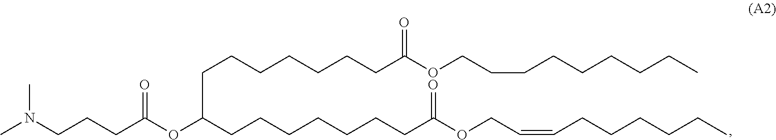 [SMILES: CCCCCC/C=C\COC(=O)CCCCCCCC(CCCCCCCC(=O)OCCCCCCCCC)OC(=O)CCCN(C)C]

or a pharmaceutically acceptable salt thereof.

In some embodiments, the lipid nanoparticle comprises a lipid having the structure:

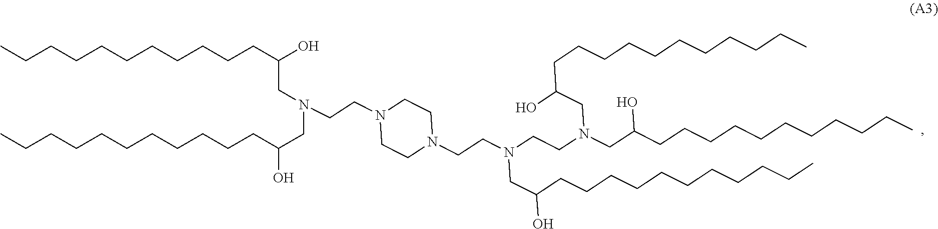 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

or a pharmaceutically acceptable salt thereof.

In some embodiments, the lipid nanoparticle comprises a lipid having the structure:

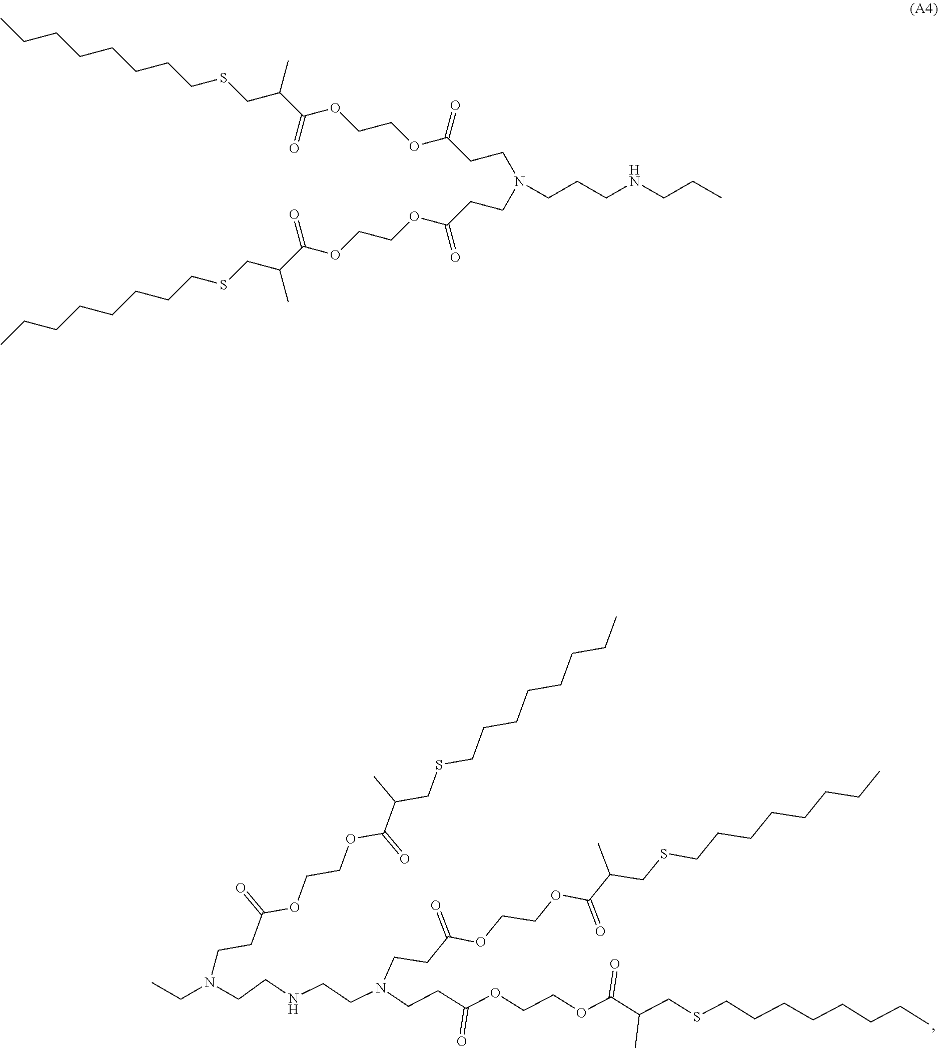

or a pharmaceutically acceptable salt thereof.

In some embodiments, the lipid nanoparticle comprises a lipid having the structure:

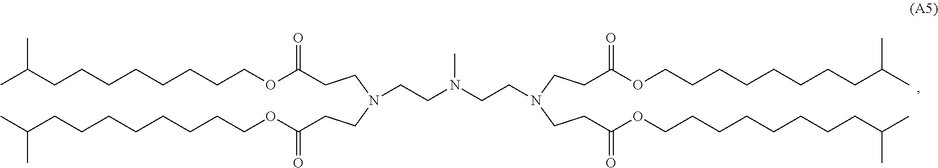 [SMILES: CC(C)CCCCCCCCOC(=O)CCN(CCC(=O)OCCCCCCCCC(C)C)CCN(C)CCN(CCC(=O)OCCCCCCCCC(C)C)CCC(=O)OCCCCCCCCC(C)C]

or a pharmaceutically acceptable salt thereof.

In some embodiments, the lipid nanoparticle comprises a lipid having the structure:

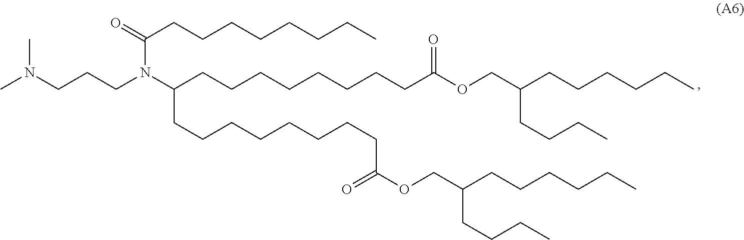 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

or a pharmaceutically acceptable salt thereof.

In some embodiments, the lipid nanoparticle comprises a lipid having the structure:

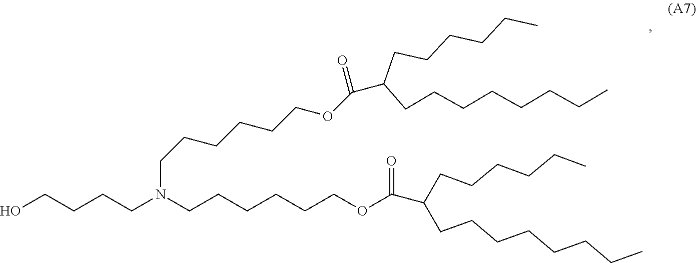 [SMILES: CCCCCCCCC(CCCCCC)C(=O)OCCCCCCN(CCCCO)CCCCCCOC(=O)C(CCCCCC)CCCCCCCC]

or a pharmaceutically acceptable salt thereof.

In some embodiments, the lipid nanoparticle comprises a lipid having the structure:

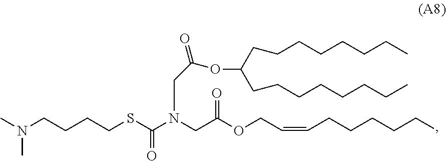

or a pharmaceutically acceptable salt thereof.

In some embodiments, the lipid nanoparticle comprises a lipid having the structure:

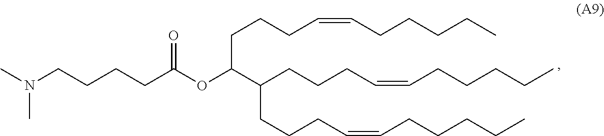 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments the lipid nanoparticle comprises a lipid having the structure:

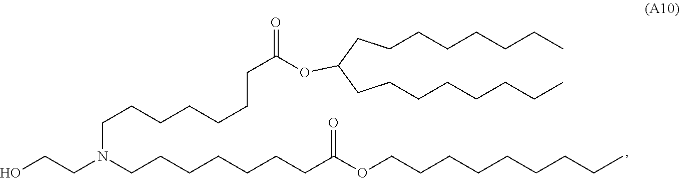 [SMILES: CCCCCCCCCOC(=O)CCCCCCCN(CCO)CCCCCCCC(=O)OC(CCCCCCCC)CCCCCCCC]

or a pharmaceutically acceptable salt thereof.

In some embodiments, the lipid nanoparticle comprises a lipid having the structure:

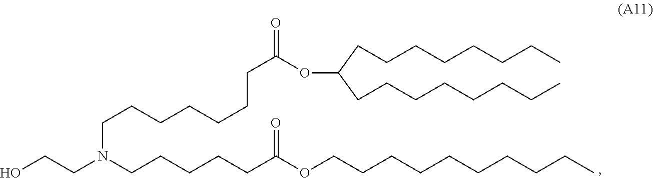 [SMILES: CCCCCCCCCCOC(=O)CCCCCN(CCO)CCCCCCCC(=O)OC(CCCCCCCC)CCCCCCCC]

or a pharmaceutically acceptable salt thereof.

### Non-Cationic Lipids

In certain embodiments, the lipid nanoparticles described herein comprise one or more non-cationic lipids. Non-cationic lipids may be phospholipids.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In certain embodiments, the lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

### Formula (HI)

In certain embodiments, a phospholipid is an analog or variant of DSPC. In certain embodiments, a phospholipid is a compound of Formula (HI):

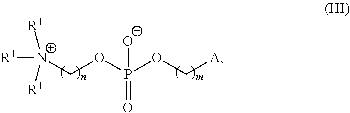

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

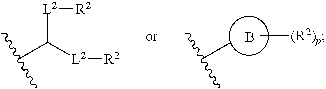

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O) OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound is not of the formula:

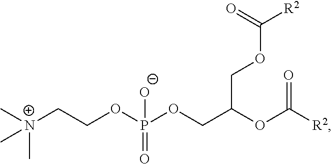

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

In some embodiments, the lipid nanoparticle comprises a molar ratio of 5-25% non-cationic lipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% non-cationic lipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% phospholipid lipid.

### Structural Lipids

The lipid composition of a pharmaceutical composition disclosed herein can comprise one or more structural lipids. As used herein, the term “structural lipid” includes sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises 30-45 mol % sterol, optionally 35-40 mol %, for example, 30-31 mol %, 31-32 mol %, 32-33 mol %, 33-34 mol %, 35-35 mol %, 35-36 mol %, 36-37 mol %, 38-38 mol %, 38-39 mol %, or 39-40 mol %. In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or 40 mol % cholesterol.

### Polyethylene Glycol (PEG)-Lipids

The lipid composition of a pharmaceutical composition disclosed herein can comprise one or more polyethylene glycol (PEG) lipids.

As used herein, the term “PEG-lipid” or “PEG-modified lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG, and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

In some embodiments, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified PEG DMG. PEG-DMG has the following structure:

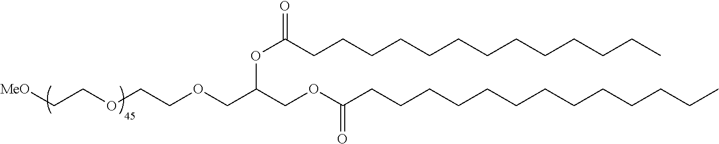

In some embodiments, PEG lipids can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

### Formula (PI)

In certain embodiments, a PEG lipid is a compound of Formula (PI):

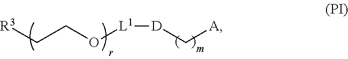

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

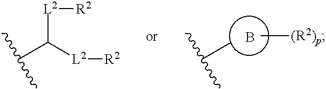

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O) OS(O) S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (PI) is a PEG-OH lipid (i.e., R³ is —OR^{O}, and R^{O} is hydrogen). In certain embodiments, the compound of Formula (PI) is of Formula (PI—OH):

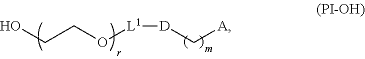

or a salt thereof.

### Formula (PII)

In certain embodiments, a PEG lipid is a PEGylated fatty acid. In certain embodiments, a PEG lipid is a compound of Formula (PII). In some embodiments, compounds of Formula (PII) have the following formula:

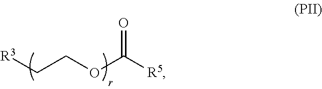

or a salts thereof, wherein:
R³ is-OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), —C(S)N(R^{N}), NR^{N}C(S), NR^{N}C(S)N(R^{N}), S(O), OS(O), S(O)O, OS(O)O, OS(O)₂, S(O)₂O, —OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (PII) is of Formula (PII—OH):

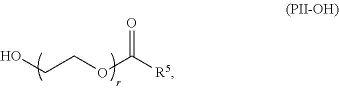

or a salt thereof. In some embodiments, r is 40-50.

In yet other embodiments the compound of Formula (PII) is:

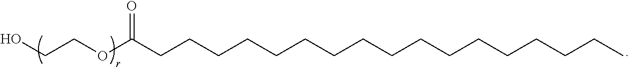

or a salt thereof.

In some embodiments, the compound of Formula (PII) is

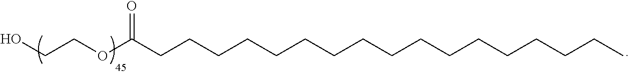

In some embodiments, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG- lipid.

In some embodiments, the lipid nanoparticle comprises 1-5% PEG-modified lipid, optionally 1-3 mol %, for example 1.5 to 2.5 mol %, 1-2 mol %, 2-3 mol %, 3-4 mol %, or 4-5 mol %. In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

Some embodiments comprise adding PEG to a composition comprising an LNP encapsulating a nucleic acid (e.g., which already includes PEG in the amounts listed above). In embodiments comprise adding about 0.5 mol % or more PEG to an LNP composition, such as about 1 mol %, about 1.5 mol %, about 2 mol %, about 2.5 mol %, about 3 mol %, about 3.5 mol %, about 4 mol %, about 5 mol %, or more after formation of an LNP composition (e.g., which already contains PEG in amount listed elsewhere herein).

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, a LNP of the disclosure comprises an ionizable amino lipid of Compound 1, wherein the non-cationic lipid is DSPC, the structural lipid that is cholesterol, and the PEG lipid is DMG-PEG.

In some embodiments, a LNP of the disclosure comprises an ionizable amino lipid of Compound 2, wherein the non-cationic lipid is DSPC, the structural lipid that is cholesterol, and the PEG lipid is DMG-PEG.

In some embodiments, a LNP comprises an ionizable amino lipid of any of Formula (AIII), (AIV), or (AV), a phospholipid comprising DSPC, a structural lipid, and a PEG lipid comprising PEG-DMG.

In some embodiments, a LNP comprises an ionizable amino lipid of any of Formula (AIII), (AIV), or (AV), a phospholipid comprising DSPC, a structural lipid, and a PEG lipid comprising a compound having Formula (PII).

In some embodiments, a LNP comprises an ionizable amino lipid of Formula (AIII), (AIV), or (AV), a phospholipid comprising a compound having Formula (HI), a structural lipid, and the PEG lipid comprising a compound having Formula (PI) or (PII).

In some embodiments, a LNP comprises an ionizable amino lipid of Formula (AIII), (AIV), or (AV), a phospholipid comprising a compound having Formula (HI), a structural lipid, and the PEG lipid comprising a compound having Formula (PI) or (PII).

In some embodiments, a LNP comprises an ionizable amino lipid of Formula (AIII), (AIV), or (AV), a phospholipid having Formula (HI), a structural lipid, and a PEG lipid comprising a compound having Formula (PII).

In some embodiments, the lipid nanoparticle comprises 49 mol % ionizable amino lipid, 10 mol % DSPC, 38.5 mol % cholesterol, and 2.5 mol % DMG-PEG.

In some embodiments, the lipid nanoparticle comprises 49 mol % ionizable amino lipid, 11 mol % DSPC, 38.5 mol % cholesterol, and 1.5 mol % DMG-PEG.

In some embodiments, the lipid nanoparticle comprises 48 mol % ionizable amino lipid, 11 mol % DSPC, 38.5 mol % cholesterol, and 2.5 mol % DMG-PEG.

In some embodiments, a LNP comprises an N:P ratio of from about 2:1 to about 30:1.

In some embodiments, a LNP comprises an N:P ratio of about 6:1.

In some embodiments, a LNP comprises an N:P ratio of about 3:1, 4:1, or 5:1.

In some embodiments, a LNP comprises a wt/wt ratio of the ionizable amino lipid component to the RNA of from about 10:1 to about 100:1.

In some embodiments, a LNP comprises a wt/wt ratio of the ionizable amino lipid component to the RNA of about 20:1.

In some embodiments, a LNP comprises a wt/wt ratio of the ionizable amino lipid component to the RNA of about 10:1.

Some embodiments comprise a composition having one or more LNPs having a diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. Some embodiments comprise a composition having a mean LNP diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. In some embodiments, the composition has a mean LNP diameter from about 30 nm to about 150 nm, or a mean diameter from about 60 nm to about 120 nm.

A LNP may comprise or one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides.

In some embodiments, the composition comprises a liposome. A liposome is a lipid particle comprising lipids arranged into one or more concentric lipid bilayers around a central region. The central region of a liposome may comprises an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles, such as those described herein, may be used for a wide variety of applications, including the stealth delivery of therapeutic payloads with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, a LNP described herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LCMS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

According to the disclosures herein, a lipid composition may comprise one or more lipids as described herein. Such lipids may include those useful in the preparation of lipid nanoparticle formulations as described above or as known in the art.

### Stabilizing Compounds

Some embodiments of the compositions described herein are stabilized pharmaceutical compositions. Various non-viral delivery systems, including nanoparticle formulations, present attractive opportunities to overcome many challenges associated with mRNA delivery. Lipid nanoparticles (LNPs) have drawn particular attention in recent years as various LNP formulations have shown promise in a variety of pharmaceutical applications. However, lipids have been shown to degrade nucleic acids, including mRNA, and lipid nanoparticle formulations undergo rapid loss of purity when stored as refrigerated liquids. Moreover, the storage stability of mRNA encapsulated within LNPs is lower than that of unencapsulated mRNA.

A class of compounds has been found to stabilize nucleic acids within a lipid carrier such as an LNP, an unexpected and unprecedented discovery which enables applications including extended refrigerated liquid shelf-life, extended in-use periods at room temperature, and extended in-use stability at physiological temperatures up to higher temperatures such as 40° C. Such stabilizing compounds solve a critical problem, as current manufacturing processes and formulations experience a 5-10% purity loss during LNP formation and processing that is typical with current large-scale LNP production.

In some embodiments, the stabilized pharmaceutical composition comprises a nucleic acid formulation comprising a nucleic acid and a stabilizing compound (e.g., a compound of Formula (I), of Formula (II), or a tautomer or solvate thereof). In some embodiments, the stabilized pharmaceutical composition comprises a nucleic acid formulation comprising a nucleic acid and a lipid, and a compound of Formula (I):

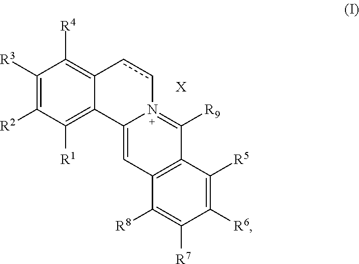

or a tautomer or solvate thereof, wherein:
[symbol] is a single bond or a double bond;
R¹ is H; R² is OCH₃, or together with R³ is OCH₂O; R³ is OCH₃, or together with R² is OCH₂O; R⁴ is H; R⁵ is H or OCH₃; R⁶ is OCH₃; R⁷ is H or OCH₃; R⁸ is H; R⁹ is H or CH₃; and X is a pharmaceutically acceptable anion, e.g., a halide such as chloride.

In some embodiments, the compound of Formula (I) has the structure of:

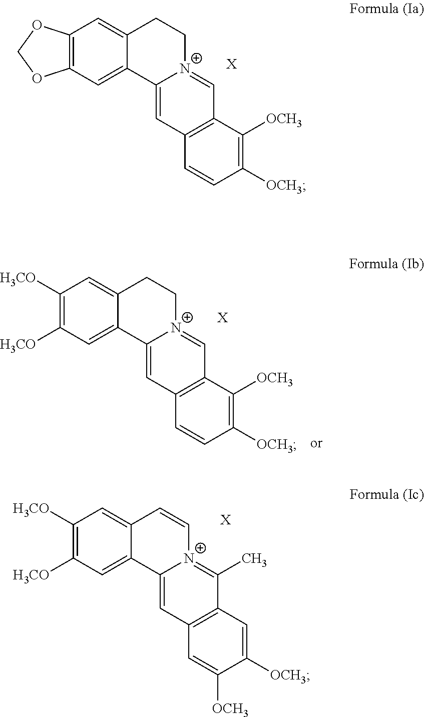

or a tautomer or solvate thereof.

In some embodiments, the stabilized pharmaceutical composition comprises a nucleic acid formulation comprising a nucleic acid and a lipid, and a compound of Formula (II):

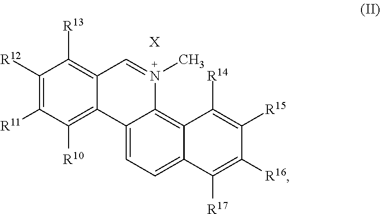

or a tautomer or solvate thereof, wherein:
R¹⁰ is H; R¹¹ is H; R¹² together with R¹³ is OCH₂O; R¹⁴ is H; R¹⁵ together with R¹⁶ is OCH₂O; R¹⁷ is H; and X is a pharmaceutically acceptable anion, e.g., a halide such as chloride.

In some embodiments, the compound of Formula (II) has the structure of:

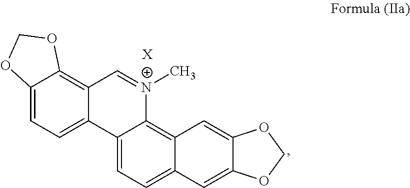

or a tautomer or solvate thereof.

Stabilizing compounds of Formulas (I), (Ia), (Ib), (Ic), (II), and (IIa) are described in International Application No. PCT/US2022/025967, which is incorporated by reference herein in its entirety.

In some embodiments, the nucleic acid formulation comprises lipid nanoparticles. In some embodiments, the nucleic acid is mRNA.

In some embodiments, the stabilizing compound (“the compound”) has a purity of at least 70%, 80%, 90%, 95%, or 99%. In some embodiments, the compound contains fewer than 100 ppm of elemental metals. In some embodiments, the stabilized pharmaceutical composition (“the composition”) comprises a pharmaceutically acceptable metal chelator, e.g., EDTA (ethylenediaminetetraacetic acid) or DTPA (diethylenetriaminepentaacetic acid).

In some embodiments, the composition is an aqueous solution. In some embodiments, the compound is present at a concentration between about 0.1 mM and about 10 mM in the aqueous solution. In some embodiments, the aqueous solution has a pH of or about 5 to 8, including pH of about 5, 5.5, 6, 6.5, 7, 7.5, or 8. In some embodiments, the aqueous solution does not comprise NaCl. In some embodiments, the aqueous solution comprises NaCl in a concentration of or about 150 mM. In some embodiments, the aqueous solution comprises a phosphate buffer, a tris buffer, an acetate buffer, a histidine buffer, or a citrate buffer.

In some embodiments, microbial growth in the composition is inhibited by the compound.

In some embodiments, the composition is characterized as having a mRNA purity level of greater than 60%, greater than 70%, greater than 80%, or greater than 90% main peak mRNA purity after at least thirty days of storage. In some embodiments, the composition comprises a mRNA purity level of greater than 50% main peak mRNA purity after at least six months of storage. In some embodiments, the storage is at room temperature.

In some embodiments, the composition comprises a lipid nanoparticle encapsulating a mRNA, and the composition comprises less than 50%, less than 60%, less than 70%, less than 80%, less than 90%, or less than 95% RNA fragments after at least thirty days of storage. In some embodiments, the storage temperature is greater than room temperature. In some embodiments, the storage temperature is about 4° C.

In some embodiments, the compound interacts with the nucleic acid comprised within a lipid nanostructure (e.g., a lipid nanoparticle, liposome, or lipoplex), e.g., via pi-pi stacking and/or by changing backbone helicity of the nucleic acid. In some embodiments, the compound intercalates with a nucleic acid. In some embodiments, the compound binds with a nucleic acid, e.g., reversible binding, and/or binding to the stranded regions of the nucleic acid. In some embodiments, the compound self-associates, binds to nucleic acid ribose contacts, and/or binds to nucleic acid base contacts. In some embodiments, the compound does not substantially bind to nucleic acid phosphate contacts. In some embodiments, the positive charge of the compound contributes to nucleic acid binding. In some embodiments, the interacts with the nucleic acid with a binding affinity defined by an equilibrium dissociation constant of less than 10⁻³ M (e.g., less than 10⁻⁴ M, less than 10⁻⁵ M, less than 10⁻⁵ M, less than 10⁻⁷ M, less than 10⁻⁸ M, or less than 10⁻⁹ M).

In some embodiments, the compound interacts with a nucleic acid and provides shielding from solvent, e.g., water. In some embodiments, the compound shields ribose from solvent more than the compound shields the phosphate groups of the nucleic acid. In some embodiments, the solvent exposure is measured by the solvent accessible surface area (SASA). In some embodiments, a stabilizing compound decreases the solvent accessible area of ribose to about 5-10 nm². In some embodiments, a stabilizing compound decreases the solvent accessible area of ribose to about 6-8 nm². In some embodiments, a stabilizing compound decreases the solvent accessible area of phosphate to about 9-12 nm². In some embodiments, a stabilizing compound decreases the solvent accessible area of phosphate to about 10-11 nm².

In some embodiments, a nucleic acid that is conformationally stabilized by the compound exhibits thermal unfolding temperatures (measured by circular dichroism or DSC, for example) that are higher than in the absence of the compound. In some embodiments, the compound confers increased stability, e.g., thermal stability, to the nucleic acid in a folded structure, e.g., relative to its unfolded or less folded or more linear form. In some embodiments, the compound causes compaction of the nucleic acid upon interaction with the nucleic acid. In some embodiments, the compound causes a decrease in the hydrodynamic radius of the nucleic acid molecule upon interaction with the nucleic acid. In some embodiments, a stabilizing compound causes compaction or a decrease in the hydrodynamic radius of a nucleic acid molecule by 5%, 10%, 15%, 20%, 25%, 30%, 35%, 40%, 45%, 50%, 55%, 60%, or more. In some embodiments, a stabilizing compound causes compaction or a decrease in the hydrodynamic radius of a nucleic acid molecule when the compound is in a concentration of 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 60 μM, 70 μM, 80 μM, 90 μM, or 100 μM.

mRNA-Lipid Adducts

It has been determined that certain ionizable lipids are susceptible to the formation of lipid-polynucleotide adducts. In particular, ionizable lipids that comprise a tertiary amine group may decompose into one or both of a secondary amine and a reactive aldehyde species capable of interacting with polynucleotides (such as mRNA) to form an ionizable lipid-polynucleotide adduct impurity that can be detected by reverse phase ion pair chromatography (RP—IP HPLC). For example, oxidation of the tertiary amine may lead to N-oxide formation that can undergo acid/base-catalyzed hydrolysis at the amine to generate aldehydes and secondary amines which may form adducts with mRNA. Thus, in some aspects, the ionizable lipid-polynucleotide adduct impurity is an aldehyde-mRNA adduct impurity.

It also has been determined that such adducts may disrupt mRNA translation and impact the activity of lipid nanoparticle (LNP) formulated mRNA products. Thus, it can be advantageous to prepare and use LNP compositions with a reduced content of ionizable lipid-polynucleotide adduct impurity, such as wherein less than about 20%, less than about 10%, less than about 5%, or less than about 1%, of the mRNA is in the form of ionizable lipid-polynucleotide adduct impurity, as may be measured by RP—IP HPLC. Thus, in accordance with some aspects, an LNP composition is provided wherein less than about 10%, less than about 5%, or less than about 1%, of the mRNA is in the form of ionizable lipid-polynucleotide adduct impurity, including less than 10%, less than 5%, or less than 1%, as may be measured by RP—IP HPLC.

In some aspects, an amount of lipid aldehydes in the composition is less than about 50 ppm, including less than 50 ppm. Additionally or alternatively, in some aspects an amount of N-oxide compounds in the composition is less than about 50 ppm, including less than 50 ppm. Additionally or alternatively, in some aspects an amount of transition metals, such as Fe, in the composition is less than about 50 ppm, including less than 50 ppm. Additionally or alternatively, in some aspects an amount of alkyl halide compounds in the composition is less than about 50 ppm, including less than 50 ppm. Additionally or alternatively, in some aspects an amount of anhydride compounds in the composition is less than about 50 ppm, including less than 50 ppm. Additionally or alternatively, in some aspects an amount of ketone compounds in the composition is less than about 50 ppm, including less than 50 ppm. Additionally or alternatively, in some aspects an amount of conjugated diene compounds in the composition is less than about 50 ppm, including less than 50 ppm.

In some aspects, the composition is stable against the formation of ionizable lipid-polynucleotide adduct impurity. In some aspects, an amount of ionizable lipid-polynucleotide adduct impurity in the composition increases at an average rate of less than about 2% per day when stored at a temperature of about 25° C. or below, including at an average rate of less than 2% per day. In some aspects, an amount of ionizable lipid-polynucleotide adduct impurity in the composition increases at an average rate of less than about 0.5% per day when stored at a temperature of about 5° C. or below, including at an average rate of less than 0.5% per day. In some aspects, an amount of ionizable lipid-polynucleotide adduct impurity in the composition increases at an average rate of less than about 0.5% per day when stored at a refrigerated temperature, optionally wherein the refrigerated temperature is about 5° C.

Lipid vehicle (e.g., LNP) compositions with a reduced content of ionizable lipid-polynucleotide adduct impurity can be prepared by methods that inhibit formation of one or both of N-oxides and aldehydes. Such methods may comprise treating a composition comprising an ionizable lipid comprising a tertiary amine group to inhibit formation of one or both of N-oxides and aldehydes, such as by treating the composition with a reducing agent; treating the composition with a chelating agent; adjusting the pH of the composition; adjusting the temperature of the composition; and adjusting the buffer in the composition. Such methods may comprise, prior to combining the ionizable lipid with a polynucleotide, one or more of treating the ionizable lipid with a scavenging agent; treating the ionizable lipid with a reductive treatment agent; treating the ionizable lipid with a reducing agent; treating the ionizable lipid with a chelating agent; treating the polynucleotide with a reducing agent; and treating the polynucleotide with a chelating agent.

In accordance with any of the foregoing, the scavenging agent, reductive treatment agent, and/or reducing agent may be an agent that reacts with aldehyde, ketone, anhydride and/or diene compounds. A scavenging agent may comprise one or more selected from (0-(2,3,4,5,6-Pentafluorobenzyl)hydroxylamine hydrochloride) (PFBHA), methoxyamine (e.g., methoxyamine hydrochloride), benzyloxyamine (e.g., benzyloxyamine hydrochloride), ethoxyamine (e.g., ethoxyamine hydrochloride), 4-[2-(aminooxy)ethyl]morpholine dihydrochloride, butoxyamine (e.g., tert-butoxyamine hydrochloride), 4-Dimethylaminopyridine (DMAP), 1,4-diazabicyclo[2.2.2]octane (DABCO), Triethylamine (TEA), Piperidine 4-carboxylate (BPPC), and combinations thereof. A reductive treatment agent may comprise a boron compound (e.g., sodium borohydride and/or bis(pinacolato)diboron). A reductive treatment agent may comprise a boron compound, such as one or both of sodium borohydride and bis(pinacolato)diboron). A chelating agent may comprise immobilized iminodiacetic acid. A reducing agent may comprise an immobilized reducing agent, such as immobilized diphenylphosphine on silica (Si-DPP), immobilized thiol on agarose (Ag-Thiol), immobilized cysteine on silica (Si-Cysteine), immobilized thiol on silica (Si-Thiol), or a combination thereof. A reducing agent may comprise a free reducing agent, such as potassium metabisulfite, sodium thioglycolate, tris(2-carboxyethyl)phosphine (TCEP), sodium thiosulfate, N-acetyl cysteine, glutathione, dithiothreitol (DTT), cystamine, dithioerythritol (DTE), dichlorodiphenyltrichloroethane (DDT), homocysteine, lipoic acid, or a combination thereof.

In accordance with any of the foregoing, the pH may be, or adjusted to be, a pH of from about 7 to about 9.

In accordance with any of the foregoing, a buffer may be selected from sodium phosphate, sodium citrate, sodium succinate, histidine, histidine-HCl, sodium malate, sodium carbonate, and TRIS (tris(hydroxymethyl)aminomethane). In accordance with any of the foregoing, a buffer may be TRIS and may be, or adjusted to be, from about 20 mM to about 150 mM TRIS.

In accordance with any of the foregoing, the temperature of the composition may be, or adjusted to be, 25° C. or less.

The composition may also comprise a free reducing agent or antioxidant.

### Multivalent Vaccines

The compositions, as provided herein, may include RNA (e.g., mRNA) or multiple RNAs (e.g., mRNAs) encoding two or more antigens of the same or different species. In some embodiments, composition includes an RNA (e.g., mRNA) or multiple RNAs (e.g., mRNAs) encoding two or more HSV proteins. In some embodiments, the RNA (e.g., mRNA) may encode 1, 2, 3, 4, 5, 6, 7, 8, 9, or 10, or more HSV proteins.

In some embodiments, two or more different RNA (e.g., mRNA) encoding antigens may be formulated in the same lipid nanoparticle. In other embodiments, two or more different RNA (e.g., mRNA) encoding antigens may be formulated in separate lipid nanoparticles (each RNA (e.g., mRNA) formulated in a single lipid nanoparticle). Lipid nanoparticles may then be combined and administered as a single vaccine composition (e.g., comprising multiple RNAs (e.g., mRNAs) encoding multiple antigens) or may be administered separately.

### Pharmaceutical Formulations

Provided herein are compositions (e.g., pharmaceutical compositions, such as vaccines), methods, kits and reagents for prevention or treatment of HSV in humans and other mammals, for example. The compositions provided herein can be used as therapeutic or prophylactic agents. They may be used in medicine to prevent and/or treat a HSV infection.

In some embodiments, the compositions containing RNA (e.g., mRNA) as described herein can be administered to a subject (e.g., a mammalian subject, such as a human subject), and the RNA (e.g., mRNA) are translated in vivo to produce an antigenic polypeptide (antigen).

An “effective amount” of a composition (e.g., comprising RNA (e.g., mRNA)) is based, at least in part, on the target tissue, target cell type, means of administration, physical characteristics of the RNA (e.g., mRNA) (e.g., length, nucleotide composition, and/or extent of modified nucleosides), other components of the vaccine, and other determinants, such as age, body weight, height, sex and general health of the subject. Typically, an effective amount of a composition provides an induced or boosted immune response as a function of antigen production in the cells of the subject. In some embodiments, an effective amount of the composition containing RNA (e.g., mRNA) having at least one chemical modification are more efficient than a composition containing a corresponding unmodified polynucleotide encoding the same antigen or a peptide antigen. Increased antigen production may be demonstrated by increased cell transfection (the percentage of cells transfected with the RNA (e.g., mRNA) vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent (e.g., mRNA) with a carrier (e.g., lipid composition, e.g., LNP)), inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the compositions (comprising polynucleotides and their encoded polypeptides) in accordance with the present disclosure may be used for treatment or prevention of a HSV infection. A composition may be administered prophylactically or therapeutically as part of an active immunization scheme to healthy individuals or early in infection during the incubation phase or during active infection after onset of symptoms. In some embodiments, the amount of RNA (e.g., mRNA) provided to a cell, a tissue or a subject may be an amount effective for immune prophylaxis.

A composition may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 12 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, or more. In exemplary embodiments, the time of administration between the initial administration of the prophylactic composition and the booster may be, but is not limited to, 1 week, 2 weeks, 3 weeks, 1 month, 2 months, 3 months, or 6 months. As is described herein, the booster may comprise the same or different RNA (e.g., mRNA) as compared to the earlier administration of the prophylactic composition. The booster, in some embodiments is monovalent (e.g., the RNA (e.g., mRNA) encodes a single antigen). In some embodiments, the booster is multivalent (e.g., the RNA (e.g., mRNA) encodes more than one antigen).

In some embodiments, “administering” or “administration” means providing a material to a subject in a manner that is pharmacologically useful. In some embodiments, a composition disclosed herein is administered to a subject enterally. In some embodiments, an enteral administration of the composition is oral. In some embodiments, a composition disclosed herein is administered to the subject parenterally. In some embodiments, a composition disclosed herein is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs.

A composition may be utilized in various settings depending on the prevalence of the infection or the degree or level of unmet medical need. As a non-limiting example, the mRNA vaccines may be utilized to treat and/or prevent HSV. mRNA vaccines have superior properties in that they produce much larger antibody titers, better neutralizing immunity, produce more durable immune responses, and/or produce responses earlier than commercially available vaccines.

Provided herein are pharmaceutical compositions including RNA (e.g., mRNA) and/or complexes optionally in combination with one or more pharmaceutically acceptable excipients.

The RNA (e.g., mRNA) may be formulated or administered alone or in conjunction with one or more other components. For example, a vaccine may comprise other components including, but not limited to, adjuvants.

In some embodiments, a vaccine does not include an adjuvant (they are adjuvant free).

An RNA (e.g., mRNA) may be formulated or administered in combination with one or more pharmaceutically-acceptable excipients. In some embodiments, vaccine compositions comprise at least one additional active substance, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, a vaccine is administered to humans, human patients or subjects. For the purposes of the present disclosure, the phrase “active ingredient” generally refers to the RNA (e.g., mRNA) contained therein, for example, RNA (e.g., mRNA) encoding HSV protein antigens.

Formulations of the vaccine compositions described herein may be prepared by any method known or hereafter developed in the art of pharmacology. In general, such preparatory methods include the step of bringing the active ingredient (e.g., mRNA) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, an RNA (e.g., mRNA) is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the RNA (e.g., mRNA) (e.g., for transplantation into a subject), hyaluronidase, nanoparticle mimics and combinations thereof.

### Methods of Prophylaxis/Treatment

Provided herein are a compositions (e.g., vaccines), methods, kits and reagents for prevention and/or treatment of a HSV infection in humans and other mammals. The compositions can be used as therapeutic or prophylactic agents. In some embodiments, the compositions are used to provide prophylactic protection from a HSV infection. In some embodiments, the compositions are used to treat a HSV infection. In some embodiments, embodiments, the compositions are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, a composition is administered to a subject (e.g., a mammalian subject, such as a human subject) in an effective amount to induce an antigen-specific immune response. The RNA encoding the HSV protein is expressed and translated in vivo to produce the antigen, which then stimulates an immune response in the subject.

Prophylactic protection from a HSV can be achieved following administration of a composition of the present disclosure. The compositions can be administered once, twice, three times, four times or more but it is likely sufficient to administer the composition once (optionally followed by a single booster). It is possible, although less desirable, to administer a composition to an infected individual to achieve a therapeutic response. Dosing may need to be adjusted accordingly.

A method of eliciting an immune response in a subject against a HSV protein (or multiple antigens) is provided in aspects of the present disclosure. In some embodiments, a method involves administering to the subject a vaccine comprising a RNA (e.g., mRNA) having an open reading frame encoding a HSV protein (or multiple antigens), thereby inducing in the subject an immune response specific to the HSV protein (or multiple antigens), wherein anti-antigen antibody titer in the subject is increased following vaccination relative to anti-antigen antibody titer in a subject vaccinated with a prophylactically effective dose of a traditional vaccine against the antigen. An “anti-antigen antibody” is a serum antibody the binds specifically to the antigen.

A prophylactically effective dose is an effective dose that prevents infection with the virus at a clinically acceptable level. In some embodiments, the effective dose is a dose listed in a package insert for the vaccine. A traditional vaccine, as used herein, refers to a vaccine other than the RNA (e.g., mRNA) vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against HSV or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against HSV or an unvaccinated subject.

A method of eliciting an immune response in a subject against HSV is provided in other aspects of the disclosure. The method involves administering to the subject a composition comprising an RNA (e.g., mRNA) comprising an open reading frame encoding a HSV protein, thereby inducing in the subject an immune response specific to HSV, wherein the immune response in the subject is equivalent to an immune response in a subject vaccinated with a traditional vaccine against HSV at 2 times to 100 times the dosage level relative to the composition.

In some embodiments, the immune response in the subject is equivalent to an immune response in a subject vaccinated with a traditional vaccine at twice the dosage level relative to a composition of the present disclosure. In some embodiments, the immune response in the subject is equivalent to an immune response in a subject vaccinated with a traditional vaccine at three times the dosage level relative to a composition of the present disclosure. In some embodiments, the immune response in the subject is equivalent to an immune response in a subject vaccinated with a traditional vaccine at 4 times, 5 times, 10 times, 50 times, or 100 times the dosage level relative to a composition of the present disclosure. In some embodiments, the immune response in the subject is equivalent to an immune response in a subject vaccinated with a traditional vaccine at 10 times to 1000 times the dosage level relative to a composition of the present disclosure. In some embodiments, the immune response in the subject is equivalent to an immune response in a subject vaccinated with a traditional vaccine at 100 times to 1000 times the dosage level relative to a composition of the present disclosure.

In other embodiments, the immune response is assessed by determining [protein] antibody titer in the subject. In other embodiments, the ability of serum or antibody from an immunized subject is tested for its ability to neutralize viral uptake or reduce HSV transformation of human B lymphocytes. In other embodiments, the ability to promote a robust T cell response(s) is measured using art recognized techniques.

Other aspects the disclosure provide methods of eliciting an immune response in a subject against HSV by administering to the subject composition comprising an mRNA having an open reading frame encoding a HSV protein, thereby inducing in the subject an immune response specific to the HSV protein, wherein the immune response in the subject is induced 2 days to 10 weeks earlier relative to an immune response induced in a subject vaccinated with a prophylactically effective dose of a traditional vaccine against HSV. In some embodiments, the immune response in the subject is induced in a subject vaccinated with a prophylactically effective dose of a traditional vaccine at 2 times to 100 times the dosage level relative to a composition of the present disclosure.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against HSV by administering to the subject an mRNA having an open reading frame encoding at least one HSV protein, wherein the mRNA does not include a stabilization element, and wherein an adjuvant is not co-formulated or co-administered with the vaccine.

A composition may be administered by any route that results in a therapeutically effective outcome. These include, but are not limited, to intradermal, intramuscular, intranasal, and/or subcutaneous administration. The present disclosure provides methods comprising administering RNA (e.g., mRNA) vaccines to a subject in need thereof. The exact amount required will vary from subject to subject, depending on the species, age, and general condition of the subject, the severity of the disease, the particular composition, its mode of administration, its mode of activity, and the like. The RNA (e.g., mRNA) is typically formulated in dosage unit form for ease of administration and uniformity of dosage. It will be understood, however, that the total daily usage of the RNA (e.g., mRNA) may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of the mRNA (e.g., an effective dose), as provided herein, may be as low as 20 μg, administered for example as a single dose or as two 10 μg doses (e.g., a first effective vaccine dose and a second effective vaccine dose). In some embodiments, the first effective vaccine dose and the second effective vaccine dose are the same amount. In some embodiments, the first effective vaccine dose and the second effective vaccine dose are different amounts. In some embodiments, the effective amount is a total dose of 5 μg-30 μg, 5 μg-25 μg, 5 μg-20 μg, 5 μg-15 μg, 5 μg-10 μg, 10 μg-30 μg, 10 μg-25 μg, 10 μg-20 μg, 10 μg-15 μg, 15 μg-30 μg, 15 μg-25 μg, 15 μg-20 μg, 20 μg-30 μg, 25 μg-30 μg, or 25 μg-300 μg. In some embodiments, the effective dose (e.g., effective amount) is at least 10 μg and less than 25 μg of the composition. In some embodiments, the effective dose (e.g., effective amount) is at least 5 μg and less than 25 μg of the composition. For example, the effective amount may be a total dose of 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. In some embodiments, the effective amount (e.g., effective dose) is a total dose of 10 μg. In some embodiments, the effective amount is a total dose of 20 μg (e.g., two 10 μg doses). In some embodiments, the effective amount is a total dose of 25 μg. In some embodiments, the effective amount is a total dose of 30 μg. In some embodiments, the effective amount is a total dose of 50 μg. In some embodiments, the effective amount is a total dose of 60 μg (e.g., two 30 μg doses). In some embodiments, the effective amount is a total dose of 75 μg. In some embodiments, the effective amount is a total dose of 100 μg. In some embodiments, the effective amount is a total dose of 150 μg. In some embodiments, the effective amount is a total dose of 200 μg. In some embodiments, the effective amount is a total dose of 250 μg. In some embodiments, the effective amount is a total dose of 300 μg.

The RNA (e.g., mRNA) described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects of the present disclosure provide formulations of the compositions (e.g., RNA vaccines), wherein the mRNA is formulated in an effective amount to produce an antigen specific immune response in a subject (e.g., production of antibodies specific to a HSV antigen). “An effective amount” is a dose of the mRNA effective to produce an antigen-specific immune response. Also provided herein are methods of inducing an antigen-specific immune response in a subject.

As used herein, an immune response to a vaccine of the present disclosure is the development in a subject of a humoral and/or a cellular immune response to a (one or more) HSV protein(s) encoded by the RNA (e.g., mRNA) present in the vaccine. For purposes of the present disclosure, a “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells. Cellular immune responses may be further divided into Th1 and Th2 responses, resulting the production of Th1-type cytokines and Th2-type cytokines, respectively. Th1-type cytokines tend to produce the proinflammatory responses responsible for killing intracellular parasites (e.g., viruses) and for perpetuating autoimmune responses. The main Th1 cytokine is interferon gamma (IFN-γ). Proinflammatory responses (e.g., Th1-based responses), in some embodiments, are counteracted by the Th2-type cytokines. The Th2-type cytokines include interleukins 4, 5, and 13, which are associated with the promotion of IgE and eosinophilic responses in atopy, and also interleukin-10, which is anti-inflammatory. In excess, Th2 responses will counteract the Th1-mediated microbicidal action. In some embodiments, Th2 responses balance excess Th1 responses to mitigate tissue damage due to inflammation. However, Th2 responses also hinder the antiviral activity of Th1 cells, which is may be deleterious in HSV infection. In some embodiments, administration of the vaccines provided herein may result in a Th17 response. T helper 17 cells (Th17) are a subset of pro-inflammatory T helper cells defined by their production of interleukin 17. Th17 cells maintain mucosal barriers and contribute to pathogen clearance at the mucosal surfaces. The Th17-type cytokines target innate immune cells and epithelial cells to produce G-CSF and I1-8, leading to neutrophil production and recruitment. In some embodiments, the compositions (e.g., vaccines) of the present disclosure produce a Th1 response. In some embodiments, the compositions (e.g., vaccines) of the present disclosure produce a Th2 response. In some embodiments, the compositions (e.g., vaccines) of the present disclosure produce a Th17 response. In some embodiments, the compositions (e.g., vaccines) of the present disclosure produce Th1 and Th2 responses, Th1 and Th17 responses, Th2 and Th17 responses, or Th1, Th2, and Th17 responses.

Some embodiments of the vaccines described herein elicit T cell responses that are polarized towards a Th1 phenotype (i.e., include more HSV-specific Th1 cells than HSV-specific Th2 cells). Polarization towards a Th1 phenotype is beneficial for preventing or treating HSV infection, at least in part because Th1 cells secrete pro-inflammatory cytokines including IFN-γ and TNF-a, which promote phagocytosis of virions and clearance of infected cells. By contrast Th2 cells and the cytokines they secrete (e.g., IL-4, IL-5, IL-9, IL-10, or IL-13) are pathogenic in HSV-2 infection, increasing morbidity and mortality rather than contributing to viral control or clearance. See, e.g., Nakajima et al., J Neuroimmunol. 2000. 110(1-2):106-113. Thus, polarizing the CD4+ T cell response towards a Th1 phenotype increases the prophylactic and/or therapeutic efficacy of the HSV vaccines described herein. In some embodiments, polarization towards a Th1 phenotype is characterized by at least 50% of CD4+ T cells specific to an HSV antigen encoded by an mRNA of the vaccine (HSV-specific CD4+ T cells) producing a Th1 cytokine (e.g., IFN-γ, TNF-a, and/or IL-2). In some embodiments, at least 50% of CD4+ T cells specific to HSV gB produce a Th1 cytokine. In some embodiments, at least 50% of CD4+ T cells specific to HSV gC produce a Th1 cytokine. In some embodiments, at least 50% of CD4+ T cells specific to HSV gD produce a Th1 cytokine. In some embodiments, at least 50%, at least 60%, at least 70%, at least 75%, at least 80%, at least 85%, at least 90%, at least 95%, or up to 100% of HSV-specific CD4+ T cells produce IFN-γ. In some embodiments, at least 50%, at least 60%, at least 70%, at least 75%, at least 80%, at least 85%, at least 90%, at least 95%, or up to 100% of HSV-specific CD4+ T cells produce TNF-a. In some embodiments, at least 50%, at least 60%, at least 70%, at least 75%, at least 80%, at least 85%, at least 90%, at least 95%, or up to 100% of HSV-specific CD4+ T cells produce IL-2. In some embodiments, polarization towards a Th1 phenotype is characterized by fewer than 50% of HSV-specific CD4+ T cells producing a Th2 cytokine (e.g., IL-4, IL-5, and/or IL-13). In some embodiments, fewer than 50%, fewer than 40%, fewer than 30%, fewer than 25%, fewer than 20%, fewer than 15%, fewer than 10%, fewer than 5%, or as few as 0% of HSV-specific CD4+ T cells produce IL-4. In some embodiments, fewer than 50%, fewer than 40%, fewer than 30%, fewer than 25%, fewer than 20%, fewer than 15%, fewer than 10%, fewer than 5%, or as few as 0% of HSV-specific CD4+ T cells produce IL-5. In some embodiments, fewer than 50%, fewer than 40%, fewer than 30%, fewer than 25%, fewer than 20%, fewer than 15%, fewer than 10%, fewer than 5%, or as few as 0% of HSV-specific CD4+ T cells produce IL-13.

In some embodiments, the antigen-specific immune response is characterized by measuring an anti-herpesvirus (e.g., anti-HSV) antigen antibody titer produced in a subject administered a composition as provided herein. An antibody titer is a measurement of the amount of antibodies within a subject, for example, antibodies that are specific to a particular antigen or epitope of an antigen. Antibody titer is typically expressed as the inverse of the greatest dilution that provides a positive result. Enzyme-linked immunosorbent assay (ELISA) is a common assay for determining antibody titers, for example.

A variety of serological tests can be used to measure antibody against encoded antigen of interest, for example, a HSV antigen. These tests include the hemagglutination-inhibition test, complement fixation test, fluorescent antibody test, enzyme-linked immunosorbent assay (ELISA), and plaque reduction neutralization test (PRNT). Each of these tests measures different antibody activities. In exemplary embodiments, A plaque reduction neutralization test, or PRNT (e.g., PRNT50 or PRNT90) is used as a serological correlate of protection. PRNT measures the biological parameter of in vitro virus neutralization and is the most serologically virus-specific test among certain classes of viruses, correlating well to serum levels of protection from virus infection. The basic design of the PRNT allows for virus-antibody interaction to occur in a test tube or microtiter plate, and then measuring antibody effects on viral infectivity by plating the mixture on virus-susceptible cells, preferably cells of mammalian origin. The cells are overlaid with a semi-solid media that restricts spread of progeny virus. Each virus that initiates a productive infection produces a localized area of infection (a plaque), that can be detected in a variety of ways. Plaques are counted and compared back to the starting concentration of virus to determine the percent reduction in total virus infectivity. In PRNT, the serum sample being tested is usually subjected to serial dilutions prior to mixing with a standardized amount of virus. The concentration of virus is held constant such that, when added to susceptible cells and overlaid with semi-solid media, individual plaques can be discerned and counted. In this way, PRNT end-point titers can be calculated for each serum sample at any selected percent reduction of virus activity.

In functional assays intended to assess vaccinal immunogenicity, the serum sample dilution series for antibody titration should ideally start below the “seroprotective” threshold titer. Regarding anti-herpesvirus (e.g., anti-HSV) neutralizing antibodies, a seropositivity threshold of 1:10 can be considered a seroprotection threshold in certain embodiments.

PRNT end-point titers are expressed as the reciprocal of the last serum dilution showing the desired percent reduction in plaque counts. The PRNT titer can be calculated based on a 50% or greater reduction in plaque counts (PRNT₅₀). A PRNT₅₀ titer is preferred over titers using higher cut-offs (e.g., PRNT₉₀, reflecting 90% or greater reduction) for vaccine sera, providing more accurate results from the linear portion of the titration curve.

There are several ways to calculate PRNT titers. The simplest and most widely used way to calculate titers is to count plaques and report the titer as the reciprocal of the last serum dilution to show >50% reduction of the input plaque count as based on the back-titration of input plaques. Use of curve fitting methods from several serum dilutions may permit calculation of a more precise result. There are a variety of computer analysis programs available for this (e.g., SPSS or GraphPad Prism).

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by an RNA vaccine.

In some embodiments, the anti-herpesvirus (e.g., anti-HSV) antigen antibody titer produced in a subject is increased by at least 1 log relative to a control. For example, the anti-herpesvirus (e.g., anti-HSV) antigen antibody titer produced in a subject may be increased by at least 1.5, at least 2, at least 2.5, or at least 3 log relative to a control. In some embodiments, the anti-herpesvirus (e.g., anti-HSV) antigen antibody titer produced in the subject is increased by 1, 1.5, 2, 2.5 or 3 log relative to a control. In some embodiments, the anti-herpesvirus (e.g., anti-HSV) antigen antibody titer produced in the subject is increased by 1-3 log relative to a control. For example, the anti-herpesvirus (e.g., anti-HSV) antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-herpesvirus (e.g., anti-HSV) antigen antibody titer produced in a subject is increased at least 2 times relative to a control. For example, the anti-herpesvirus (e.g., anti-HSV) antigen antibody titer produced in a subject may be increased at least 3 times, at least 4 times, at least 5 times, at least 6 times, at least 7 times, at least 8 times, at least 9 times, or at least 10 times relative to a control. In some embodiments, the anti-herpesvirus (e.g., anti-HSV) antigen antibody titer produced in the subject is increased 2, 3, 4, 5, 6, 7, 8, 9, or 10 times relative to a control. In some embodiments, the anti-herpesvirus (e.g., anti-HSV) antigen antibody titer produced in a subject is increased 2-10 times relative to a control. For example, the anti-herpesvirus (e.g., anti-HSV) antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

In some embodiments, an antigen-specific immune response is measured as a ratio of geometric mean titer (GMT), referred to as a geometric mean ratio (GMR), of serum neutralizing antibody titers to a herpesvirus (e.g., HSV). A geometric mean titer (GMT) is the average antibody titer for a group of subjects calculated by multiplying all values and taking the nth root of the number, where n is the number of subjects with available data.

A control, in some embodiments, is an anti-herpesvirus (e.g., anti-HSV) antigen antibody titer produced in a subject who has not been administered an RNA (e.g., mRNA) vaccine. In some embodiments, a control is an anti-herpesvirus (e.g., anti-HSV) antigen antibody titer produced in a subject administered a recombinant or purified protein vaccine. Recombinant protein vaccines typically include protein antigens that either have been produced in a heterologous expression system (e.g., bacteria or yeast) or purified from large amounts of the pathogenic organism.

In some embodiments, the ability of an RNA (e.g., mRNA) vaccine to be effective is measured in a murine model. For example, a composition may be administered to a murine model and the murine model assayed for induction of neutralizing antibody titers. Bacterial challenge studies may also be used to assess the efficacy of a vaccine of the present disclosure. For example, a composition may be administered to a murine model, the murine model challenged with virus, and the murine model assayed for survival and/or immune response (e.g., neutralizing antibody response, T cell response (e.g., cytokine response)).

In some embodiments, an effective amount of an RNA (e.g., mRNA) vaccine is a dose that is reduced compared to the standard of care dose of a recombinant protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified protein vaccine, or a live attenuated or inactivated vaccine, or a VLP vaccine, that a physician/clinician or other medical professional would administer to a subject to treat or prevent HSV infections or a related condition, while following the standard of care guideline for treating or preventing HSV infection or a related condition.

In some embodiments, the anti-herpesvirus (e.g., anti-HSV) antigen antibody titer produced in a subject administered an effective amount of a composition is equivalent to the anti-herpesvirus (e.g., anti-HSV) antigen antibody titer produced in a control subject administered a standard of care dose of a recombinant or purified protein vaccine, or a live attenuated or inactivated vaccine, or a VLP vaccine.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and

Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In some embodiments, efficacy of an RNA (e.g., mRNA) vaccine is at least 60% relative to unvaccinated control subjects. For example, efficacy of the composition may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective pathogen infection into the host. In some embodiments, the effective amount of a composition of the present disclosure is sufficient to provide sterilizing immunity in the subject for at least 1 year. For example, the effective amount of a composition of the present disclosure is sufficient to provide sterilizing immunity in the subject for at least 2 years, at least 3 years, at least 4 years, at least 5 years, at least 6 years, at least 7 years, at least 8 years, at least 9 years, at least 10 years, or more. In some embodiments, the effective amount of a composition of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of a composition of the present disclosure is sufficient to produce detectable levels of HSV antigen as measured in serum of the subject at 1-72 hours post administration.

Titer. An antibody titer is a measurement of the number of antibodies within a subject, for example, antibodies that are specific to a particular antigen (e.g., an anti-HSV antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of a composition of the present disclosure is sufficient to produce a 1,000-10,000 neutralizing antibody titer produced by neutralizing antibody against the specific antigen as measured in serum of the subject at 1-72 hours post administration. In some embodiments, the effective amount is sufficient to produce a 1,000-5,000 neutralizing antibody titer produced by neutralizing antibody against the specific antigen as measured in serum of the subject at 1-72 hours post administration. In some embodiments, the effective amount is sufficient to produce a 5,000-10,000 neutralizing antibody titer produced by neutralizing antibody against the specific antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-herpesvirus (e.g., anti-HSV) antigen antibody titer produced in the subject is increased by at least 1 log relative to a control. For example, an anti-herpesvirus (e.g., anti-HSV) antigen antibody titer produced in the subject may be increased by at least 2, 3, 4, 5, 6, 7, 8, 9 or 10 log relative to a control.

In some embodiments, an anti-herpesvirus (e.g., anti-HSV) antigen antibody titer produced in the subject is increased at least 2 times relative to a control. For example, an anti-herpesvirus (e.g., anti-HSV) antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a recombinant protein vaccine or an attenuated virus vaccine.

### Additional Embodiments

Additional embodiments of the present disclosure are encompassed by the following numbered paragraphs:

1. A herpes simplex virus (HSV) messenger ribonucleic acid (mRNA) vaccine comprising
(a) an mRNA comprising an open reading frame encoding a herpes simplex virus (HSV) glycoprotein B (gB);
(b) an mRNA comprising an open reading frame encoding an HSV glycoprotein C (gC);
(c) an mRNA comprising an open reading frame encoding an HSV glycoprotein D (gD); and
(d) an mRNA comprising an open reading frame encoding an HSV intracellular protein (ICP) selected from the group consisting of HSV ICP0 and HSV ICP4.

2. The vaccine of paragraph 1, wherein the HSV gB comprises a proline stabilizing mutation or is truncated at its C-terminus, relative to an HSV gB comprising a wild-type amino acid sequence of HSV gB such as SEQ ID NO: 36.

3. A herpes simplex virus (HSV) messenger ribonucleic acid (mRNA) vaccine comprising
(a) an mRNA comprising an open reading frame encoding a herpes simplex virus (HSV) glycoprotein B (gB), wherein the HSV gB comprises a proline stabilizing mutation or is truncated at its C-terminus, relative to an HSV gB comprising a wild-type amino acid sequence HSV gB such as SEQ ID NO: 36;
(b) an mRNA comprising an open reading frame encoding an HSV glycoprotein C (gC); and
(c) an mRNA comprising an open reading frame encoding an HSV glycoprotein D (gD).

4. The vaccine of paragraph 2 or 3, wherein the HSV gB comprises a proline stabilizing mutation.

5. The vaccine of paragraph 4, wherein the proline stabilizing mutation is at a position corresponding to position H510 of an HSV gB protein comprising the amino acid sequence of SEQ ID NO: 36.

6. The vaccine of paragraph 5, wherein the HSV gB comprises an amino acid sequence having at least 80%, least 85%, least 90% or at least 95% identity to the amino acid sequence of SEQ ID NO: 37, and wherein the proline stabilizing mutation is at a position corresponding to H510.

7. The vaccine of any one of paragraphs 2-4, wherein the HSV gB comprises two proline stabilizing mutations.

8. The vaccine of paragraph 7, wherein the proline stabilizing mutations are at positions corresponding to:
(a) positions 1518 and A519;
(b) positions M514 and L515;
(c) positions H510 and V511; or
(d) positions H506 and 1507 of an HSV gB protein comprising the amino acid sequence of SEQ ID NO: 36.

9. The vaccine of any one of paragraphs 1-8, wherein the HSV gB comprises an amino acid sequence having at least 80%, at least 85%, at least 90%, at least 95% or 100% identity to the amino acid sequence of any one of SEQ ID NOs: 50-53.

10. The vaccine of any one of paragraphs 2-8, wherein the HSV gB is truncated at its C-terminus, relative to an HSV gB comprising a wild-type amino acid sequence HSV gB such as SEQ ID NO: 35.

11. The vaccine of paragraph 9, wherein the HSV gB has a length of no longer than 850, 840, 830, 820, 810, 800, or 799 amino acids.

12. The vaccine of paragraph 9 or 10, wherein the HSV gB comprises an amino acid sequence having at least 80%, at least 85%, at least 90%, at least 95% or 100% identity to the amino acid sequence of any one of SEQ ID NOs: 54-57.

13. The vaccine of any one of paragraphs 3-12, wherein the vaccine comprises an mRNA comprising an open reading frame encoding an HSV intracellular protein (ICP) selected from the group consisting of HSV ICP0 and HSV ICP4.

14. The vaccine of paragraph 1 or 13, wherein the vaccine comprises an mRNA comprising an open reading frame encoding an HSV ICP0, and an mRNA comprising an open reading frame encoding an HSV ICP4.

15. The vaccine of any one of paragraphs 1 or 13-14, wherein the HSV ICP0 comprises an amino acid sequence having at least 80%, at least 85%, at least 90%, at least 95% or 100% identity to the amino acid sequence of SEQ ID NO: 46.

16. The vaccine of any one of paragraphs 1 or 13-15, wherein the HSV ICP0 lacks a nuclear localization signal and/or a RING finger domain.

17. The vaccine of paragraph 16, wherein the HSV ICP0 comprises an amino acid sequence having at least 80%, at least 85%, at least 90%, at least 95% or 100% identity to the amino acid sequence of SEQ ID NO: 47.

18. The vaccine of any one of paragraphs 1 or 13-17, wherein the HSV ICP4 comprises an amino acid sequence having at least 80%, at least 85%, at least 90%, at least 95% or 100% identity to the amino acid sequence of SEQ ID NO: 48.

19. The vaccine of paragraphs 1 or 13-18, wherein the HSV ICP4 lacks a nuclear localization signal and/or a RING finger domain.

20. The vaccine of paragraph 19, wherein the HSV ICP4 comprises an amino acid sequence having at least 80%, at least 85%, at least 90%, at least 95% or 100% identity to the amino acid sequence of SEQ ID NO: 49.

21. The vaccine of any one of the preceding paragraphs, wherein the HSV gC comprises a mutation in a complement-binding domain.

22. The vaccine of paragraph 21, wherein the mutation in the complement-binding domain is an amino acid substitution at a position corresponding to position F327 of an HSV gC protein comprising the amino acid sequence of SEQ ID NO: 65.

23. The vaccine of paragraph 22, wherein the HSV gC comprises an amino acid sequence having at least 80%, least 85%, least 90% or at least 95% identity to the amino acid sequence of SEQ ID NO: 38, and wherein the mutation is an amino acid substitution at position F327.

24. The vaccine of paragraph 22 or 23, wherein the amino acid substitution is F327A.

25. The vaccine of any one of paragraphs 1-20, wherein the HSV gC comprises an amino acid sequence having at least 80%, at least 85%, at least 90%, at least 95% or 100% identity to the amino acid sequence of SEQ ID NO: 38.

26. The vaccine of any one of paragraph 21-25, wherein the binding affinity of the HSV gC for human C3b protein is at least 1.5, 2.0, 2.5, 3.0, 3.5, 4.0, 4.5, 5.0, 6.0, 7.0, 8.0, 9.0, or 10.0 times lower than the binding affinity of an HSV gC comprising the amino acid sequence of SEQ ID NO: 65.

27. The vaccine of any one of the preceding paragraphs, wherein the HSV gD comprises an amino acid sequence having at least 80%, at least 85%, at least 90%, at least 95% or 100% identity to the amino acid sequence of SEQ ID NO: 65.

28. The vaccine of any one of the preceding paragraphs, further comprising an mRNA comprising an open reading frame encoding an HSV glycoprotein E (gE).

29. The vaccine of paragraph 28, wherein the HSV gE comprises an amino acid sequence having at least 80%, at least 85%, at least 90%, at least 95% or 100% identity to the amino acid sequence of SEQ ID NO: 41.

30. The vaccine of paragraph 28, wherein the HSV gE is soluble.

31. The vaccine of paragraph 30, wherein the HSV gE is truncated at its C-terminus, relative to an HSV gE comprising a wild-type amino acid sequence of HSV gE such as SEQ ID NO: 41.

32. The vaccine of paragraph 31, wherein the HSV gE has a length of no longer than 450, 440, 430, 420, 410, or 400 amino acids.

33. The vaccine of paragraph 30 or 31, wherein the HSV gE comprises an amino acid sequence having at least 80%, at least 85%, at least 90%, at least 95% or 100% identity to the amino acid sequence of SEQ ID NO: 41.

34. The vaccine of any one of the preceding paragraphs, wherein the molar ratio of the mRNA of (a) to the mRNA of (b) to the mRNA of (c) is 1:1:1 or 3:1:1.

35. The vaccine of any one of paragraphs 1-33, wherein the mass ratio of the mRNA of (a) to the mRNA of (b) to the mRNA of (c) is 1:1:1 or 3:1:1.

36. The vaccine of any one of the preceding paragraphs, wherein the mRNA of (a), the mRNA of (b), and/or the mRNA of (c) comprises a chemical modification.

37. The vaccine of paragraph 36, wherein 100% of the uracil of the mRNA of (a), the mRNA of (b), and/or the mRNA of (c) comprises the chemical modification.

38. The vaccine of paragraph 36 or 37, wherein the chemical modification is 1-methylpseudouracil.

39. The vaccine of any one of the preceding paragraphs, wherein the mRNAs are in a lipid nanoparticle.

40. The vaccine of paragraph 39, wherein the lipid nanoparticle comprises an ionizable lipid, a neutral lipid, a sterol, and a PEG-modified lipid.

41. A method comprising administering to a subject the vaccine of any one of the preceding paragraphs, wherein the subject has an HSV infection, has been exposed to HSV, or is at risk of HSV infection.

42. The method of paragraph 41, wherein the vaccine is administered in an amount effective for preventing a latent HSV infection in the subject.

43. The method of paragraph 41, wherein the vaccine is administered in an amount effective for preventing reactivation of a latent HSV infection in the subject.

44. The method of paragraph 41, wherein the vaccine is administered in an amount effective for preventing replication of HSV.

45. The method of paragraph 41, wherein the vaccine is administered in an amount effective for reducing duration of an HSV infection in the subject.

46. The method of paragraph 41, wherein the vaccine is administered in an amount effective for reducing the number of replication-competent HSV particles in the subject.

47. The method of paragraph 41, wherein the vaccine is administered in an amount effective for reducing the number of cells in the subject comprising an HSV genome.

48. The method of paragraph 41, wherein the vaccine is administered in an amount effective for inducing a T cell-mediated immune response in the subject.

49. The method of paragraph 48, wherein the T cell-mediated immune response comprises IFN-γ+CD4+ T cells, IL-2+CD4+ T cells, and/or TNF-α+CD4+ T cells.

50. A herpes simplex virus (HSV) protein comprising an amino acid sequence having at least 90%, at least 95%, at least 96%, at least 97%, at least 98%, at least 99%, or 100% identity to the amino acid sequence of any one of SEQ ID NOs: 36-70.

51. A ribonucleic acid (RNA) comprising an open reading frame encoding the HSV protein of paragraph 50.

52. A ribonucleic acid (RNA) comprising an open reading frame comprising a nucleic acid sequence having at least 90%, at least 95%, at least 96%, at least 97%, at least 98%, at least 99%, or 100% identity to the amino acid sequence of any one of SEQ ID NOs: 1-35.

53. The RNA of paragraph 51 or 52, wherein the RNA is a messenger RNA.

54. The RNA of any one of paragraphs 51-53, wherein the RNA is in a lipid nanoparticle.

55. The RNA of paragraph 54, wherein the lipid nanoparticle comprises a cationic lipid, a neutral lipid, a sterol, and a PEG-modified lipid.

### EXAMPLES

### Methods

According to the present disclosure, the manufacture of polynucleotides and/or parts or regions thereof may be accomplished utilizing the methods taught in International Publication WO2014/152027, entitled “Manufacturing Methods for Production of RNA Transcripts,” the content of which is incorporated herein by reference in its entirety.

Purification methods may include those taught in International Publication WO2014/152030 and International Publication WO2014/152031, each of which is incorporated herein by reference in its entirety.

Detection and characterization methods of the polynucleotides may be performed as taught in International Publication WO2014/144039, which is incorporated herein by reference in its entirety.

Characterization of the polynucleotides of the disclosure may be accomplished using polynucleotide mapping, reverse transcriptase sequencing, charge distribution analysis, detection of RNA impurities, or any combination of two or more of the foregoing. “Characterizing” comprises determining the RNA transcript sequence, determining the purity of the RNA transcript, or determining the charge heterogeneity of the RNA transcript, for example. Such methods are taught in, for example, International Publication WO2014/144711 and International Publication WO2014/144767, the contents of each of which are incorporated herein by reference in their entirety.

Production, purification, and evaluation of mRNA vaccines encoding HSV antigens may be accomplished using methods described in International Publication WO 2017/070623 and International Publication WO 2018/170256, the contents of each of which are incorporated herein by reference in their entirety.

In experiments where a lipid nanoparticle (LNP) formulation was used, the formulation included 48 mol % ionizable lipid of Compound 1, 11 mol % 1,2 distearoyl-sn-glycero-3-phosphocholine (DSPC), 38.5 mol % cholesterol, and 2.5 mol % PEG-modified 1,2 dimyristoyl-sn-glycerol, methoxypolyethyleneglycol (PEG2500 DMG).

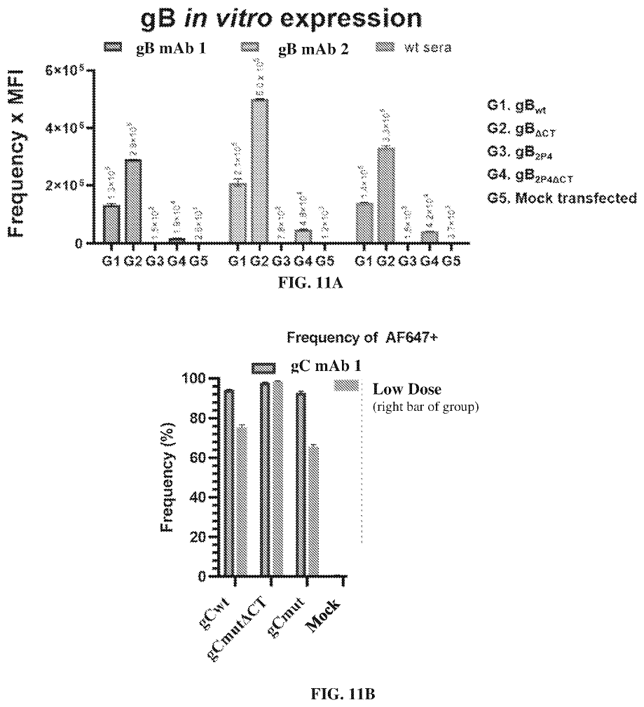

mRNAs encoding one or more HSV proteins, including glycoprotein D, glycoprotein B, glycoprotein C, glycoprotein E, glycoprotein H, glycoprotein L, intracellular protein 0 (ICP0), and/or intracellular protein (ICP4) were generated and encapsulated in lipid nanoparticles (FIG. 12A). The HSV glycoprotein B encoded was either a wild-type glycoprotein B (gBwt), or a glycoprotein B comprising an H510P substitution to stabilize the gB in its prefusion conformation (gBpf). To confirm that this substitution did not interfere with immunogenicity, HEK293 cells were transfected with mRNA encoding either gB, incubated with diluted (1:100, 1:1,000, or 1:10,000) sera from mice immunized with PBS control, mRNA encoding gBwt, or mRNA encoding gBpf, and then stained with Alexa Fluor 647-labeled anti-mouse IgG to detect mouse antibodies bound to the cells. Cells transfected with either mRNA were efficiently labeled by sera, particularly more concentrated sera diluted only 100-fold (FIG. 12B), and 10,000-fold diluted sera from mice immunized with either mRNA labeled all cells with comparable mean fluorescence intensities (FIG. 12C). These results indicate that many epitopes are shared between gBwt and gBpf, allowing mRNA encoding HSV gBpf to generate antibodies specific to wild-type HSV gB proteins.

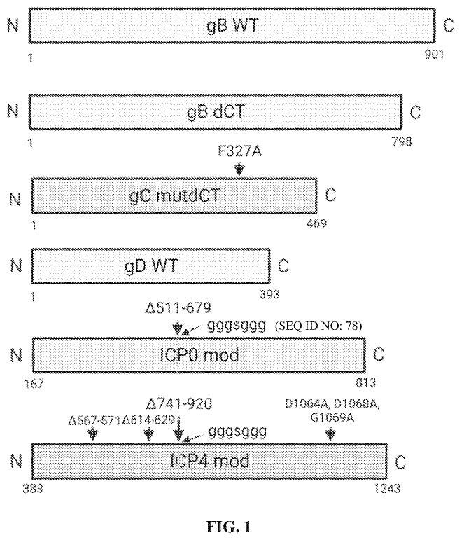

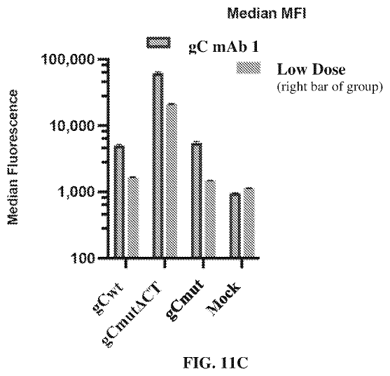

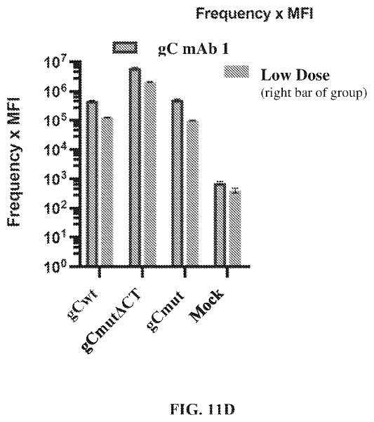

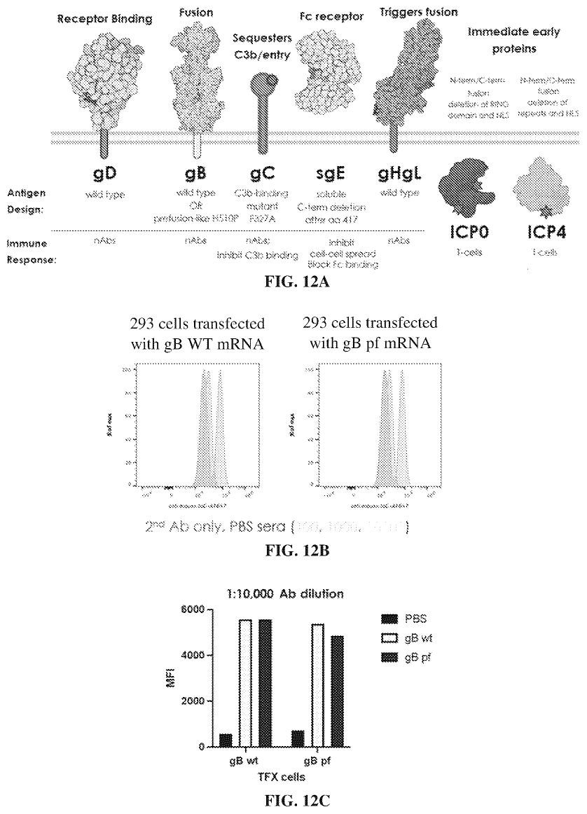

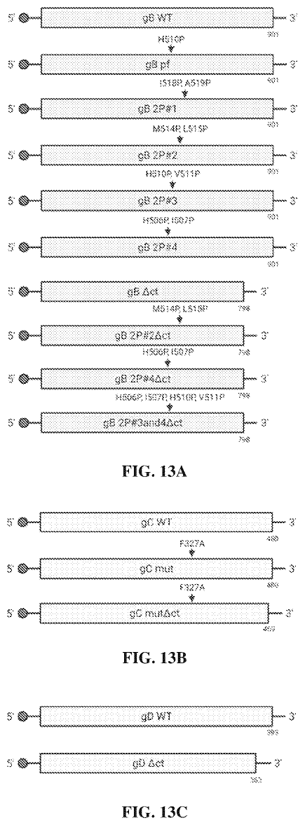

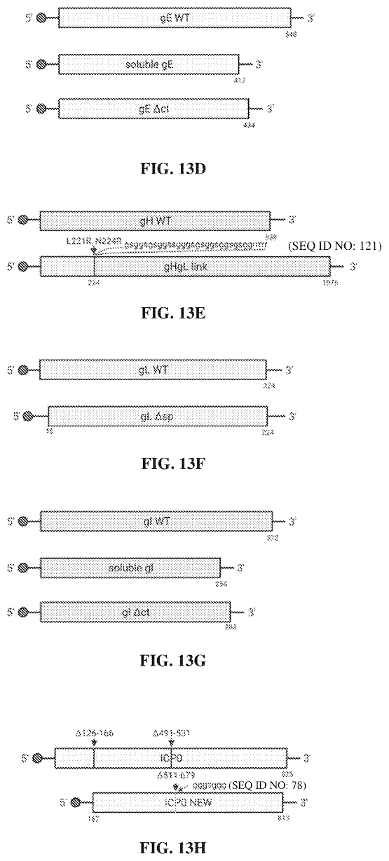

mRNAs encoding wild-type and variant versions of HSV surface proteins and glycoproteins including gB, gC, gD, gE, gH, gL, and gHgL were generated to test the effects of mutations, deletions, truncations, and linkers on immunogenicity. In additional intracellular proteins 0 and 4 (ICP0 and ICP4) were extensively modified with mutations, deletions, and linkers, and evaluated for immunogenicity. The mutations present in each of these variant proteins are shown in FIGS. 1, 4, and 13A-13I. Variant HSV gB proteins contained the H510P substitution described above, 2 proline substitutions at indicated positions, truncation of the cytoplasmic tail, and/or multiple proline substitutions and deletion of the cytoplasmic tail (FIGS. 1, 4, 13A). Variant gC proteins contained an F327A mutation to abrogate binding of gC to human complement protein C3b, and optionally deletion of the cytoplasmic tail of gC (FIGS. 1, 4, 13B). Variant gD proteins contained a deletion of the cytoplasmic tail of gD (FIG. 13C). Variant gE proteins were either soluble or contained a deletion of the cytoplasmic tail of gE (FIG. 13D). Variant gH proteins contained a fusion of gH to a gL domain, with the gH and gL being joined by a flexible linker (FIG. 13E). Variant gL proteins contained a deletion of the signal peptide (FIG. 13F). Variant gI proteins were soluble or contained a deletion of the cytoplasmic tail of gI (FIG. 13G). Variant HSV ICP0 proteins contained a deletion of two amino acid sequences 42 amino acids long, or deletion of the N-terminal 166 amino acids and an additional 170 internal amino acids (FIGS. 1, 4, 13H). Variant HSV ICP4 proteins contained multiple amino acid substitutions, and optionally deletion of the N-terminal 382 amino acids and an additional 181 internal amino acids (FIGS. 1, 4, 13I).

### Immunization Methods

Vaccine compositions of lipid nanoparticles containing mRNAs were administered to mice according the following administration schedule. C57BL/6 mice were immunized with two doses of a given composition, receiving the first dose on day 0, and the second dose on day 22. Sera were collected on day 21, three weeks after the first (prime) dose but before administration of the second (boost) dose, and day 36, two weeks after administration of the second (boost) dose. Where T cells were evaluated, mice were euthanized on day 36, and spleens were collected and processed to harvest splenocytes. Splenocytes were stimulated with one of a panel of peptide pools, each pool containing peptides from a single HSV antigen, in the presence of a Golgi blocker so that cells producing cytokines in response to stimulation would retain cytokines instead of secreting them. Cell surfaces were stained for lymphocyte markers, including CD3, CD4, and CD8, and cells were permeabilized and stained for multiple cytokines. Stained cells were incubated with a viability dye and analyzed by flow cytometry.

### Neutralization Assays

Antibodies in serum, when bound to a viral surface protein that is essential for infection, can prevent a virus from infecting a target cell, an activity referred to as “neutralization.” To determine the ability of mRNA compositions to generate neutralizing antibodies against HSV-1 and/or HSV-2, the neutralization activity of sera was quantified using a neutralization assay. For each assay, ARPE-19 cells were plated in 96-well plates, at a density of 2*10⁴ cells/well and incubated for 20-24 hours. Then, serial 3-fold dilutions of each serum sample were prepared in phenol red-free cDMEM. A consistent amount of HSV (HSV-1 or HSV-2) reporter virus, containing a gene encoding GFP, was incubated with each serum dilution sample, to allow for binding of any HSV-specific antibodies to the virus. After incubation, cells were washed, and incubated with HSV/serum mixtures. 24 hours after incubation, GFP fluorescence in each well was measured to determine the extent of infection. For a given serum sample, the 50% neutralization titer (NT₅₀) was calculated as the reciprocal of the serum dilution at which 50% of GFP+ cells were observed.

### Antibody-Dependent Cell-Mediated Cytotoxicity Assays

Antibodies in serum, when bound to viral protein expressed on the surface of an infected cell, can be recognized by effector cells, such as natural killer (NK) cells. Effector cells recognize the constant (Fc) region of antibodies bound to the target cells, and following recognition, release cytotoxic granules that induce apoptosis in the infected target cells. This process is referred to as “antibody-dependent cell-mediated cytotoxicity” (ADCC). To determine the ability of mRNA compositions to generate antibodies that could facilitate ADCC, the ADCC activity of sera was quantified using an ADCC assay. This assay used Jurkat cells that constitutively express mouse FcγRIV, allowing for recognition of antibody Fc regions, and luciferase under the control of the NFAT pathway, which is activated following Fc recognition. In each assay, Vero cells were plated in 96-well plates, at a density of 2.5*10⁴ cells/well, incubated for 20-24 hours, then inoculated with HSV-1 at a multiplicity of infection (MOI) of 5 plaque-forming units (PFU) per cell. 16 hours after inoculation, serial 3-fold dilutions of serum samples were prepared in RPMI+4% fetal bovine serum (FBS) containing only minimal amounts of IgG, to reduce background. Serum samples were added to each well, to allow antibodies to bind to infected Vero cells expressing viral surface proteins. Then, reporter effector cells were serially diluted in RPMI+4% low-IgG FBS, added to wells, and incubated for 6 hours to allow for recognition of surface-bound antibodies and expression of luciferase. After the 6 hours of incubation, a luciferase substrate was added to wells, so that any luciferase present would react with the substrate to produce light. Light emitted from wells was measured to quantify the amount of luciferase activity as a measurement of ADCC activity.

### Example 1: Immunization of Mice with Compositions Containing mRNAs Encoding HSV Glycoproteins and Intracellular Proteins at Escalating Doses

Mice were immunized with lipid nanoparticles containing the mRNAs encoding gB, gC, gD, and optionally ICP0 and ICP4 variant proteins, shown in FIG. 1. The antigens encoded by the mRNAs of each composition are shown in Table 4. A first dose (prime) was administered on day 0, and a second dose (booster) was administered on day 22. Serum was collected on day 21, three weeks after the administration after the first dose but before booster dose administration, and on day 36, two weeks after the administration of the booster dose. At day 36, mice were also euthanized to collect spleens for analysis of T cells by cell surface marker and intracellular cytokine staining. Sera were evaluated for antiviral activities such as neutralization, ADCC activity, inhibition of complement C3b-binding by HSV gC, and prevention of cell-cell spread by HSV.

TABLE 4
Panel of mRNA vaccines containing varying doses of mRNAs
encoding HSV glycoproteins and intracellular proteins.

| | | Total mRNA dose | Sample size | |
|---|---|---|---|---|
| Group | HSV antigens | (ratio of mRNAs) | (# of mice) | SEQ ID NO: |
| 1 | None (PBS) | — | 8 | — |
| 2 | gBΔCT, gCmutΔCT, gD, | 5 μg | 8 | 19, 28, 4, 12, 14 |
| | ICP0 (variant), ICP4 (variant) | (38:20:20:7.5:14.5) | | (mRNA) |
| | | | | 54, 63, 39, 47, 49 |
| | | | | (protein) |
| 3 | gBΔCT, gCmutΔCT, gD, | 10 μg | 8 | 19, 28, 4, 12, 14 |
| | ICP0 (variant), ICP4 (variant) | (38:20:20:7.5:14.5) | | (mRNA) |
| | | | | 54, 63, 39, 47, 49 |
| | | | | (protein) |
| 4 | gBwt, gCmutΔCT, gD, ICP0 | 5 μg | 8 | 1, 28, 4, 12, 14 |
| | (variant), ICP4 (variant) | (38:20:20:7.5:14.5) | | (mRNA) |
| | | | | 36, 63, 39, 47, 49 |
| | | | | (protein) |
| 5 | gBΔCT, gCmutΔCT, gD | 3.9 μg | 8 | 19, 28, 4 |
| | | (38:20:20) | | (mRNA) |
| | | | | 54, 63, 39 |
| | | | | (protein) |
| 6 | gBΔCT, gCmutΔCT, gD | 7.8 μg | 8 | 19, 28, 4 |
| | | (38:20:20) | | (mRNA) |
| | | | | 54, 63, 39 |
| | | | | (protein) |
| 7 | gBΔCT, gCmutΔCT, gD | 3.9 μg | 8 | 19, 28, 4 |
| | | (38:20:20) | | (mRNA) |
| | | | | 54, 63, 39 |
| | | | | (protein) |

Δ = d = deletion;
CT = cytoplasmic tail

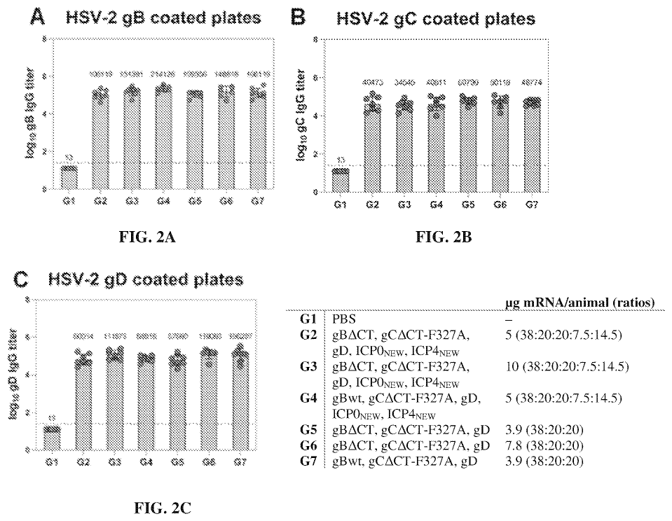

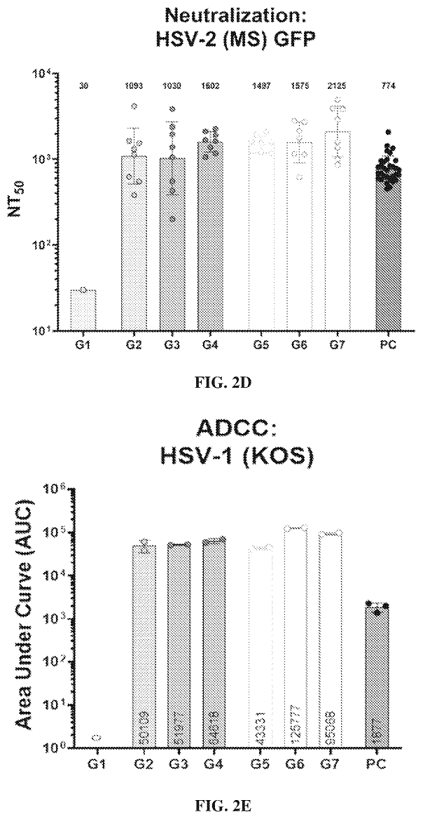

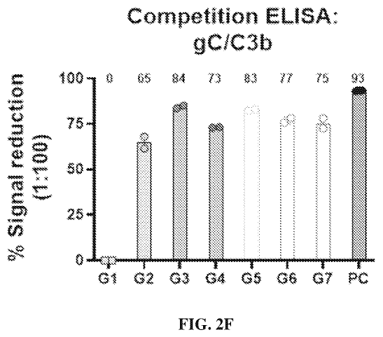

For each glycoprotein, antigen-specific antibody titers were similar in all groups after administration of compositions containing an approximately 2:1:1 mass ratio (38:20:20) of encoding HSV ICP0 and ICP4 did not substantially reduce the generation of glycoprotein-specific antibody titers (FIGS. 2A-2C, comparing G2 v. G5 and G3 v. G6).

With respect to neutralizing antibody titers, the highest NT₅₀ titers were observed in mice administered compositions containing only mRNAs encoding gB, gC, and gD (FIG. 2D, G7), but all mice vaccinated with mRNA compositions exhibited higher NT50 titers than positive control high titer by about 1.5-fold (FIG. 2D, comparing G2 v. G4 and G5 v. G7). Overall, variation between groups were minimal (<2-fold difference).

With respect to ADCC activity, the highest NT50 titers were again observed in mice administered compositions containing only mRNAs encoding gB, gC, and gD, but the reduction in signal from the inclusion of mRNAs encoding ICP0 and ICP4 was minimal (FIG. 2E, G2 v. G5 and G3 v. G6), with the largest difference between groups being less than 3-fold. As with neutralization titers, all mice vaccinated with mRNA compositions exhibited sera with substantially higher ADCC activity than positive control human serum.

Immunization with mRNAs encoding gC containing a truncated cytoplasmic tail elicited antibodies that were capable of inhibiting the C3b-binding activity of gC in a functional assay evaluating gC/C3b-binding activity (FIG. 2F, G2-G7 v. sham-vaccinated mice of G1). Additionally, including ICP0 and ICP4-encoding mRNAs had little impact on functional antibody responses), with no substantial differences being observed between groups of mice vaccinated with mRNA compositions.

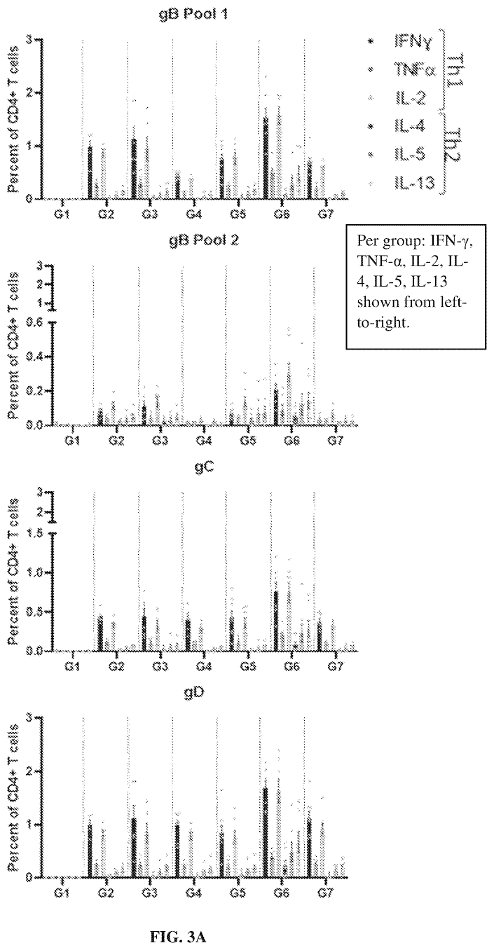

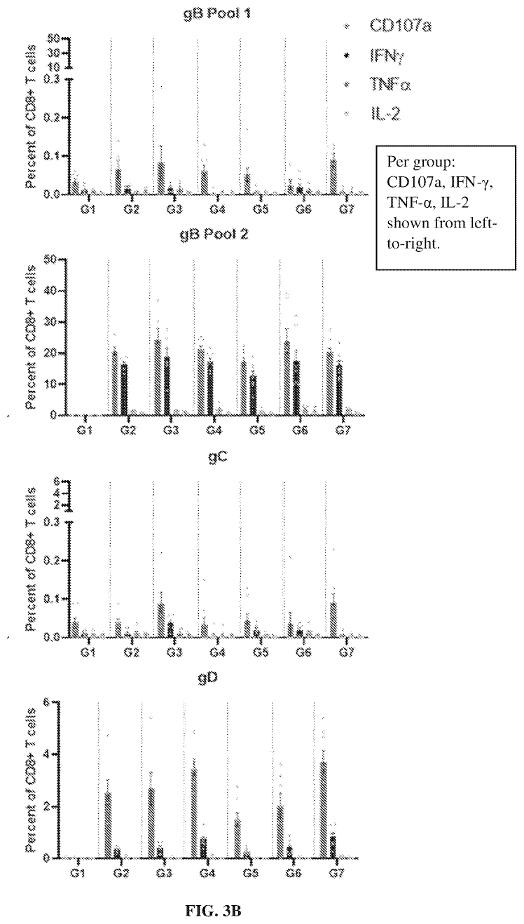

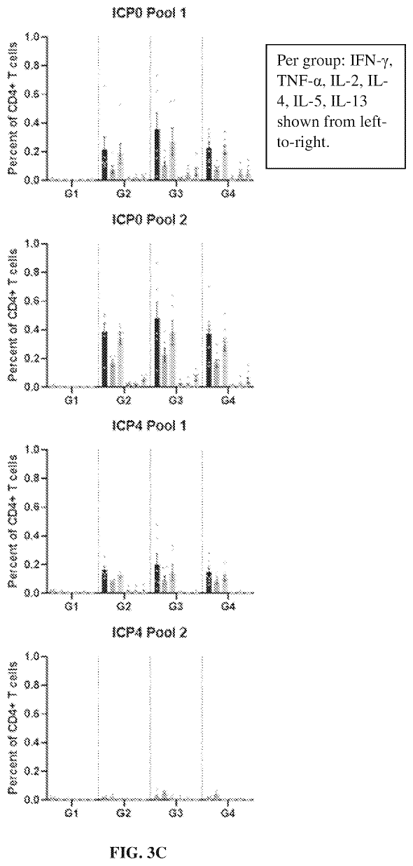

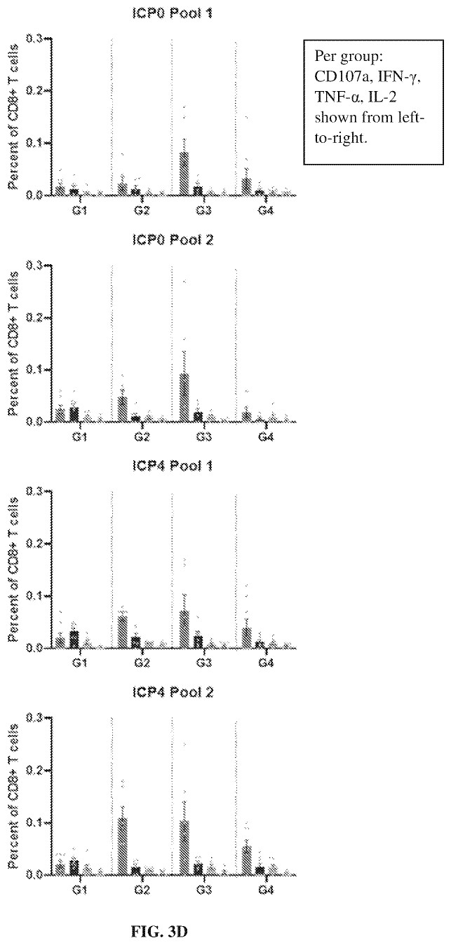

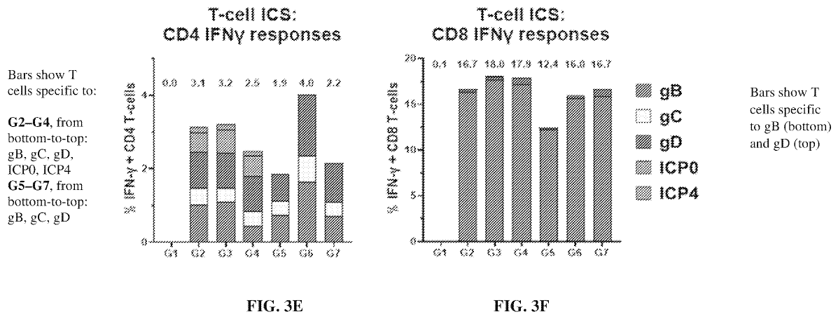

With respect to T cell responses, robust CD4+ T cell responses were observed to peptides of HSV gB, gC, and gD in all immunized groups, and responses to peptides of HSV ICP0 and ICP4 were observed in all groups immunized with compositions containing mRNAs encoding those antigens (FIGS. 3A, 3C, 3E). While inclusion of mRNAs encoding ICP0 and ICP4 in compositions modestly reduced Th1 cell (expressing IFN-γ, TNF-α, and/or IL-2) response to glycoprotein peptides, their presence also limited the generation of Th2 cells (expressing IL-4, IL-5, and IL-13) (FIGS. 3A, 3C). As Th2 cells and associated cytokines IL-4, IL-5, and IL-13 are associated with increased morbidity and mortality in HSV-2 infection, the ability of compositions containing mRNAs encoding HSV gB, gC, gD, ICP0, and ICP4 to elicit an antiviral Th1 response while limiting the pathogenic Th2 response is useful for preventing and controlling HSV infection.

Additionally, robust CD8+ T cell responses were observed to peptides of all antigens except HSV gC (FIGS. 3B, 3D, 3F). CD8+ T cell responses were similar across vaccine groups, and dominated by gB-specific CD8+ T cells (FIG. 3F). This phenomenon is believed to arise from a CD8+ T cell epitope that (gB₄₉₈₋₅₀₅) that is immunodominant in C57BL/6J mice.

### Example 2: Immunization of Mice with Compositions Containing mRNAs Encoding HSV Glycoproteins and Intracellular Proteins at Escalating Doses

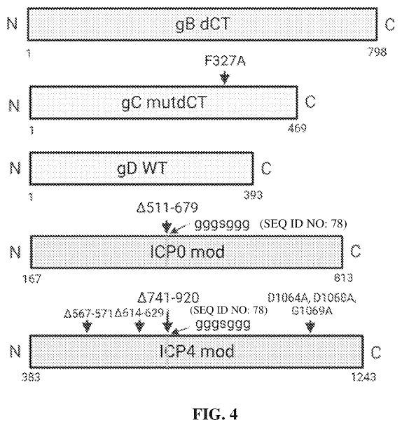

Mice were immunized with lipid nanoparticles containing mRNAs encoding gBΔCT, gCmutΔCT, gD, variant ICP0, and variant ICP4, shown in FIG. 4. The total mass of mRNA in each dose varied from 0.625 μg to 10 μg per mouse. The mRNA doses, and ratios between mRNAs, in each composition are shown in Table 5. A first dose (prime) was administered on day 0, and a second dose (booster) was administered on day 22. Sera were collected on day 21, three weeks after the administration after the first dose but before booster dose administration, and on day 36, two weeks after the administration of the booster dose. At day 36, mice were also euthanized to collect spleens for analysis of T cells by cell surface marker and intracellular cytokine staining. Sera were evaluated for antiviral activities such as neutralization, ADCC activity, and inhibition of complement C3b-binding by HSV gC.

TABLE 5
Panel of mRNA vaccines containing varying doses of mRNAs
encoding HSV glycoproteins and intracellular proteins.

| | | Total mRNA dose | Sample size | |
|---|---|---|---|---|
| Group | HSV antigens | (ratio of mRNAs) | (# of mice) | SEQ ID NO: |
| 1 | None (PBS) | — | 8 | — |
| 2 | gBΔCT, | 0.625 μg | 8 | 85 + 19 + 88 (gB 5′ UTR + ORF + 3′ UTR), |
| | gCmutΔCT, gD, | (38:20:20:7.5:14.5) | | 85 + 28 + 89 (gC 5′ UTR + ORF + 3′ UTR), |
| | ICP0 (variant), | | | 85 + 4 + 90 (gD 5′ UTR + ORF + 3′ UTR), |
| | ICP4 (variant) | | | 85 + 12 + 91 (ICP0 5′ UTR + ORF + 3′ UTR), |
| | | | | 85 + 14 + 92 (ICP4 5′ UTR + ORF + 3′ UTR); |
| | | | | 104-108 (full-length mRNAs) |
| | | | | 54, 63, 39, 47, 49 (protein) |
| 3 | gBΔCT, | 1.25 μg | 8 | 85 + 19 + 88 (gB 5′ UTR + ORF + 3′ UTR), |
| | gCmutΔCT, gD, | (38:20:20:7.5:14.5) | | 85 + 28 + 89 (gC 5′ UTR + ORF + 3′ UTR), |
| | ICP0 (variant), | | | 85 + 4 + 90 (gD 5′ UTR + ORF + 3′ UTR), |
| | ICP4 (variant) | | | 85 + 12 + 91 (ICP0 5′ UTR + ORF + 3′ UTR), |
| | | | | 85 + 14 + 92 (ICP4 5′ UTR + ORF + 3′ UTR); |
| | | | | 104-108 (full-length mRNAs) |
| | | | | 54, 63, 39, 47, 49 (protein) |
| 4 | gBΔCT, | 2.5 μg | 8 | 85 + 19 + 88 (gB 5′ UTR + ORF + 3′ UTR), |
| | gCmutΔCT, gD, | (38:20:20:7.5:14.5) | | 85 + 28 + 89 (gC 5′ UTR + ORF + 3′ UTR), |
| | ICP0 (variant), | | | 85 + 4 + 90 (gD 5′ UTR + ORF + 3′ UTR), |
| | ICP4 (variant) | | | 85 + 12 + 91 (ICP0 5′ UTR + ORF + 3′ UTR), |
| | | | | 85 + 14 + 92 (ICP4 5′ UTR + ORF + 3′ UTR); |
| | | | | 104-108 (full-length mRNAs) |
| | | | | 54, 63, 39, 47, 49 (protein) |
| 5 | gBΔCT, | 5.0 μg | 8 | 85 + 19 + 88 (gB 5′ UTR + ORF + 3′ UTR), |
| | gCmutΔCT, gD, | (38:20:20:7.5:14.5) | | 85 + 28 + 89 (gC 5′ UTR + ORF + 3′ UTR), |
| | ICP0 (variant), | | | 85 + 4 + 90 (gD 5′ UTR + ORF + 3′ UTR), |
| | ICP4 (variant) | | | 85 + 12 + 91 (ICP0 5′ UTR + ORF + 3′ UTR), |
| | | | | 85 + 14 + 92 (ICP4 5′ UTR + ORF + 3′ UTR); |
| | | | | 104-108 (full-length mRNAs) |
| | | | | 54, 63, 39, 47, 49 (protein) |
| 6 | gBΔCT, | 10 μg | 8 | 85 + 19 + 88 (gB 5′ UTR + ORF + 3′ UTR), |
| | gCmutΔCT, gD, | (38:20:20:7.5:14.5) | | 85 + 28 + 89 (gC 5′ UTR + ORF + 3′ UTR), |
| | ICP0 (variant), | | | 85 + 4 + 90 (gD 5′ UTR + ORF + 3′ UTR), |
| | ICP4 (variant) | | | 85 + 12 + 91 (ICP0 5′ UTR + ORF + 3′ UTR), |
| | | | | 85 + 14 + 92 (ICP4 5′ UTR + ORF + 3′ UTR); |
| | | | | 104-108 (full-length mRNAs) |
| | | | | 54, 63, 39, 47, 49 (protein) |

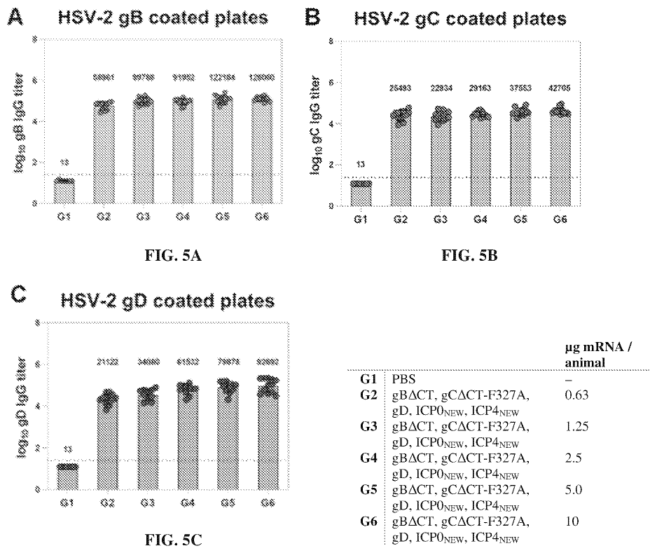

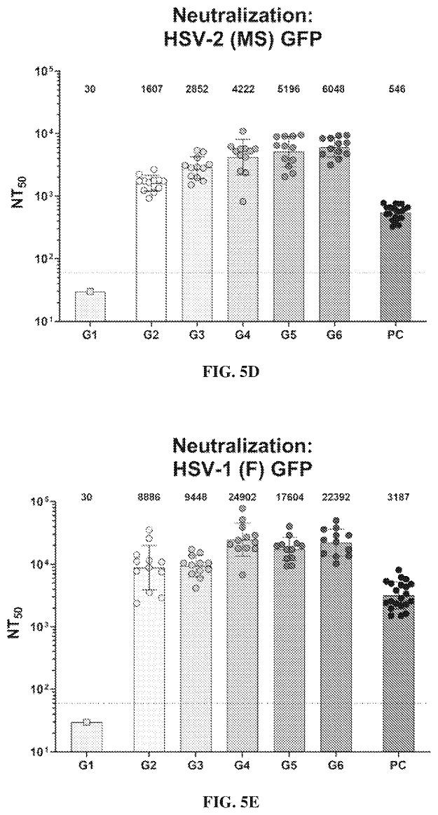

With respect to glycoprotein-specific antibody responses, all doses of the mRNA vaccine composition elicited strong binding antibody responses to each of gB, gC, and gD (FIGS. 5A-5C). While some dose-dependence was observed, binding antibody titers are high regardless of dose for each glycoprotein.

With respect to neutralizing antibody responses, immunization with increasing doses of the mRNA composition showed a dose-dependent neutralizing antibody response that was able to inhibit infection by HSV-2 (FIG. 5D) and cross-neutralize against HSV-1 infection (FIG. 5E). All dose levels outperformed high titer positive control human serum (PC), which was run on each assay plate.

With respect to ADCC activity, sera from all groups of vaccinated mice similarly outperformed positive control serum, in particular doses of 2.5-10 μg (FIG. 5F, G2-G6, PC v. sham-vaccinated mice of G1).

With respect to C3b-binding inhibition, sera from all groups of vaccinated mice reduced C3b-binding by HSV gC, with higher doses increasing the competitive inhibition (FIG. 5G, G2-G6, PC v. sham-vaccinated mice of G1). Sera from all vaccinated mice outperformed positive control serum (PC). PC serum was generated by immunization of mice with LNPs containing 5 μg mRNA encoding gC-F327A with an intact cytoplasmic tail, while, as noted in Table 5, mice in this study were immunized using mRNAs encoding gCΔCT-F327A. The ability of even low doses of mRNAs encoding gCΔCT-F327A to generate sera that more effectively inhibits the C3b-binding activity of HSV gC further demonstrates the improvement in expression achieved by truncation of the HSV gC cytoplasmic tail.

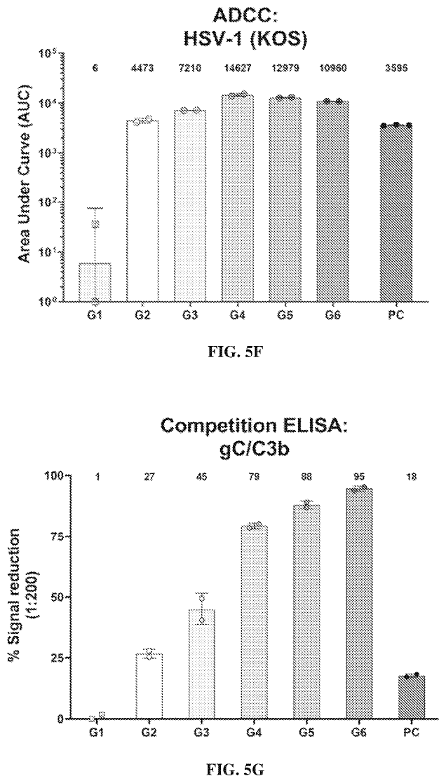

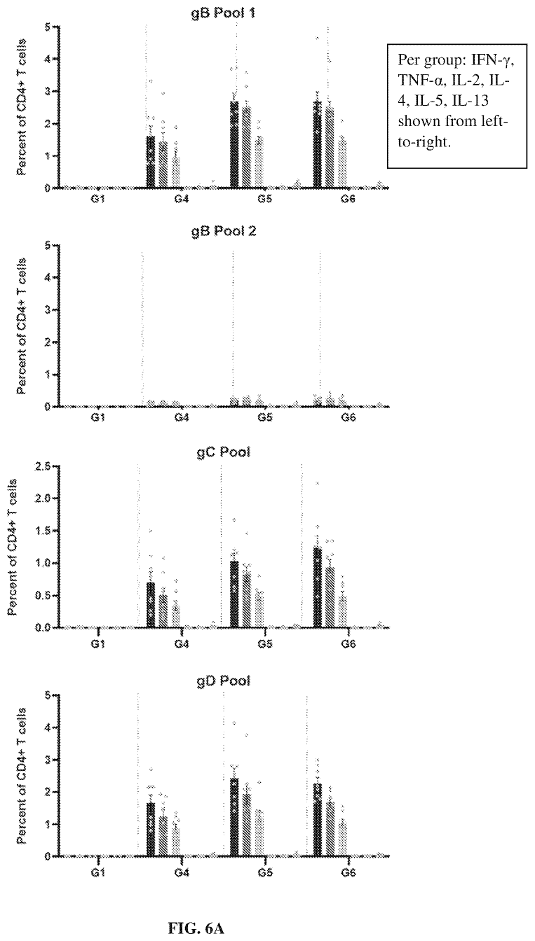

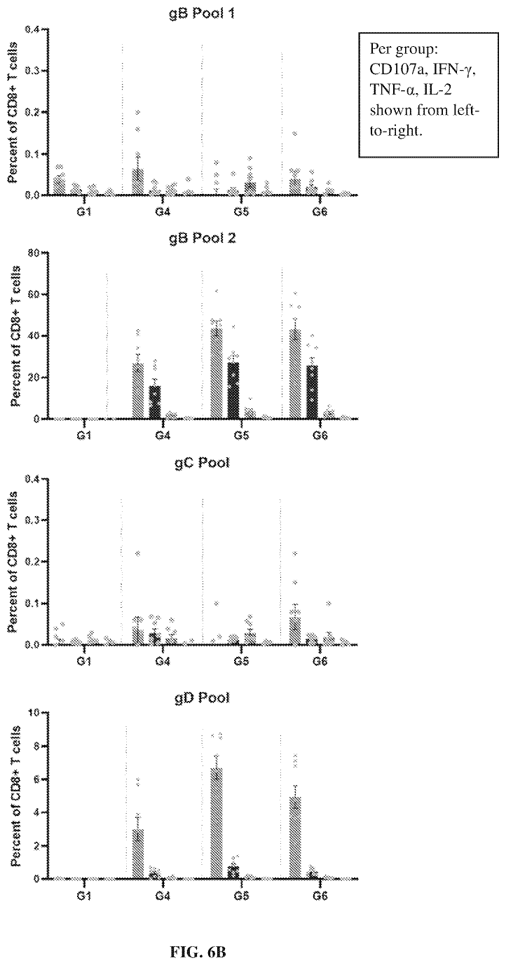

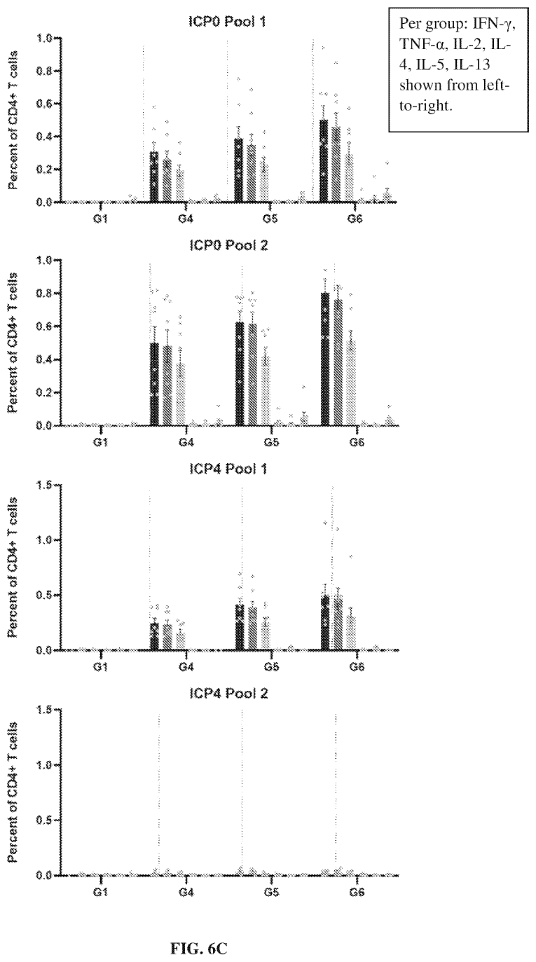

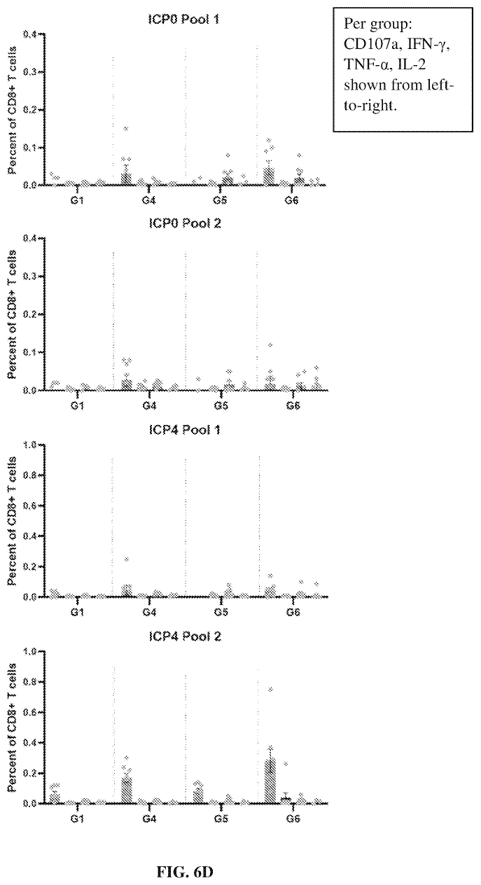

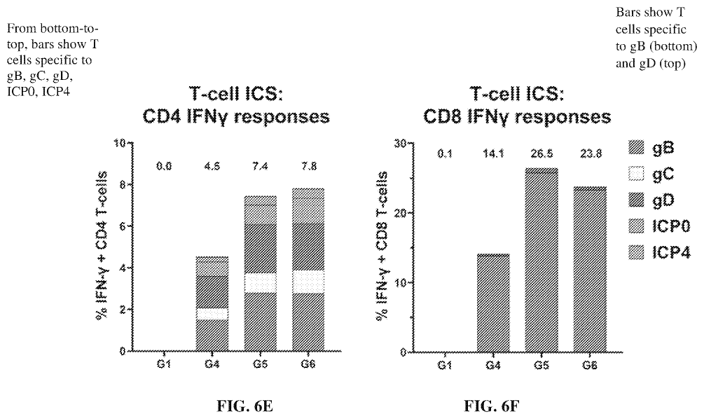

With respect to T cell responses, CD4+ T cell responses increased with mRNA dose, with a plateau being reached for the glycoprotein antigens at the 5 μg mRNA/animal dose (FIGS. 6A, 6C). At all doses, the population of HSV-specific CD4+ T cells was skewed towards a Th1 phenotype, as minimal to no Th2 cytokines (e.g., IL-4, IL-5, IL-13) were observed for any antigen (FIGS. 6A, 6C, 6E). CD8+ T cell responses increased with dose up to 5 μg mRNA/animal, with a slight decrease in the proportion of CD8+ T cells that produced IFN-γ at 10 μg/animal doses (FIGS. 6B, 6D, 6F). CD8+ T cell responses were dominated by gB-specific T cells, as expected in C57BL/6J mice due to the presence of the immunodominant CD8+ T cell epitope gB₄₉₈₋₅₀₅.

In another experiment, mice were immunized with lipid nanoparticles containing the mRNAs encoding gB, gC, gD, ICP0, and ICP4 variant proteins. The antigens encoded by the mRNAs of each composition are shown in Table 6. In a first group of compositions, mRNAs contained 5′ and 3′ UTRs of SEQ ID NO: 86. In a second group, mRNAs encoding the same antigens contained different 5′ and 3′ UTRs of SEQ ID NOs: 88-92. A first dose (prime) was administered on day 0, and a second dose (booster) was administered on day 22. Sera were collected on day 21, three weeks after the administration after the first dose but before booster dose administration, and on day 36, two weeks after the administration of the booster dose. At day 36, mice were also euthanized to collect spleens for analysis of T cells by cell surface marker and intracellular cytokine staining. Sera were evaluated for antiviral activities such as neutralization, ADCC activity, inhibition of complement C3b-binding by HSV gC.

TABLE 6
Panel of mRNA vaccines containing varying doses of mRNAs encoding HSV glycoproteins
and intracellular proteins with varying 5′ UTRs and 3′ UTRs.

| | | mRNA dose | Sample size | |
|---|---|---|---|---|
| Group | HSV antigens | (μg mRNA per antigen) | (# of mice) | SEQ ID NO: |
| 1 | None (PBS) | — | 8 | — |
| 2 | gBΔCT, | 1 | 8 | 84 + 19 + 87 (gB 5′ UTR + ORF + 3′ UTR), |
| | gCmutΔCT, gD, | (38:20:20:7.5:14.5), | | 84 + 28 + 87 (gC 5′ UTR + ORF + 3′ UTR), |
| | ICP0 (variant), | v1.1 UTRs | | 84 + 4 + 87 (gD 5′ UTR + ORF + 3′ UTR), |
| | ICP4 (variant) | | | 84 + 12 + 87 (ICP0 5′ UTR + ORF + 3′ UTR), |
| | | | | 84 + 14 + 87 (ICP4 5′ UTR + ORF + 3′ UTR) |
| | | | | 54, 63, 39, 47, 49 (protein) |
| 3 | gBΔCT, | 5 | 8 | 84 + 19 + 87 (gB 5′ UTR + ORF + 3′ UTR), |
| | gCmutΔCT, gD, | (38:20:20:7.5:14.5), | | 84 + 28 + 87 (gC 5′ UTR + ORF + 3′ UTR), |
| | ICP0 (variant), | v1.1 UTRs | | 84 + 4 + 87 (gD 5′ UTR + ORF + 3′ UTR), |
| | ICP4 (variant) | | | 84 + 12 + 87 (ICP0 5′ UTR + ORF + 3′ UTR), |
| | | | | 84 + 14 + 87 (ICP4 5′ UTR + ORF + 3′ UTR) |
| | | | | 54, 63, 39, 47, 49 (protein) |
| 4 | gBΔCT, | 1 | 8 | 85 + 19 + 88 (gB 5′ UTR + ORF + 3′ UTR), |
| | gCmutΔCT, gD, | (38:20:20:7.5:14.5) | | 85 + 28 + 89 (gC 5′ UTR + ORF + 3′ UTR), |
| | ICP0 (variant), | v2.0 UTRs | | 85 + 4 + 90 (gD 5′ UTR + ORF + 3′ UTR), |
| | ICP4 (variant) | | | 85 + 12 + 91 (ICP0 5′ UTR + ORF + 3′ UTR), |
| | | | | 85 + 14 + 92 (ICP4 5′ UTR + ORF + 3′ UTR); |
| | | | | 104-108 (full-length mRNAs) |
| | | | | 54, 63, 39, 47, 49 (protein) |
| 5 | gBΔCT, | 5 | 8 | 85 + 19 + 88 (gB 5′ UTR + ORF + 3′ UTR), |
| | gCmutΔCT, gD, | (38:20:20:7.5:14.5), | | 85 + 28 + 89 (gC 5′ UTR + ORF + 3′ UTR), |
| | ICP0 (variant), | v2.0 UTRs | | 85 + 4 + 90 (gD 5′ UTR + ORF + 3′ UTR), |
| | ICP4 (variant) | | | 85 + 12 + 91 (ICP0 5′ UTR + ORF + 3′ UTR), |
| | | | | 85 + 14 + 92 (ICP4 5′ UTR + ORF + 3′ UTR); |
| | | | | 104-108 (full-length mRNAs) |
| | | | | 54, 63, 39, 47, 49 (protein) |

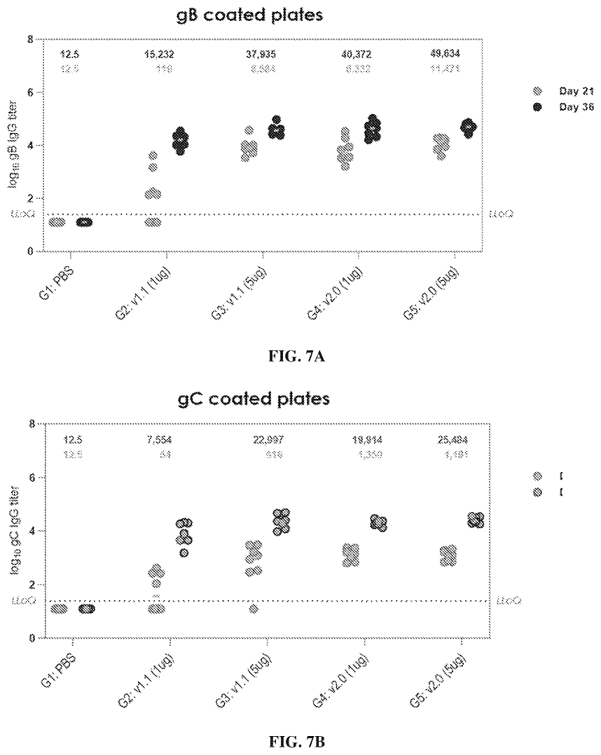

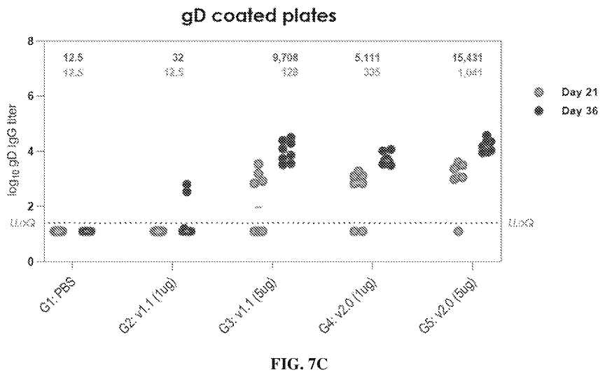

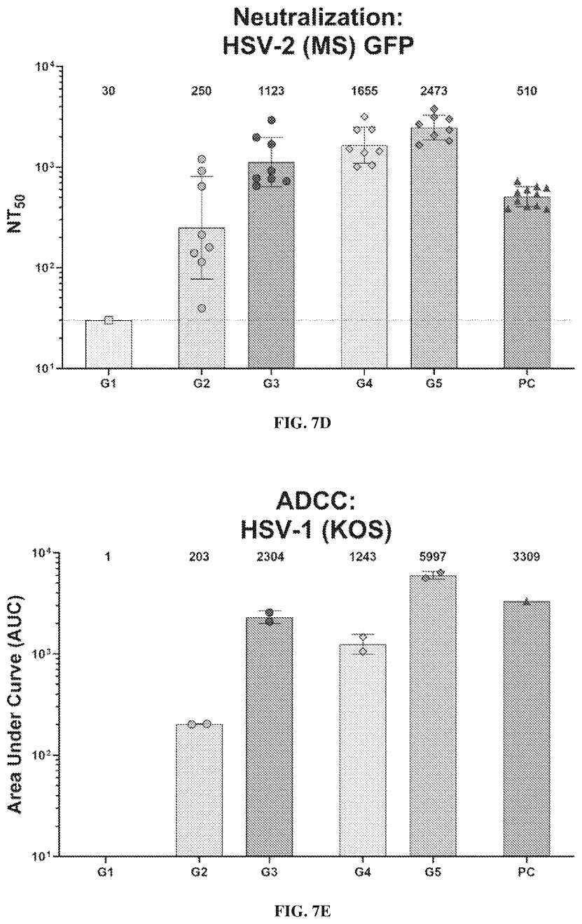

With respect to binding antibody responses, the mRNA vaccine compositions elicited strong binding antibody responses to each of gB, gC, and gD (FIGS. 7A-7C). Use of v2.0 5′ and 3′ UTRs improved binding antibody titers towards each glycoprotein, with this increase being greater in magnitude at low doses (FIGS. 7A-7C, G2 v. G4 and G3 v. G5).

With respect to functional antibody responses, use of v2.0 5′ and 3′ UTRs markedly increased NT₅₀ titers, with a greater increase being observed at lower total mRNA doses (FIG. 7D, G2 v. G4 and G3 v. G5). A similar trend was observed for ADCC activity (FIG. 7E, G2 v. G4 and G3 v. G5).

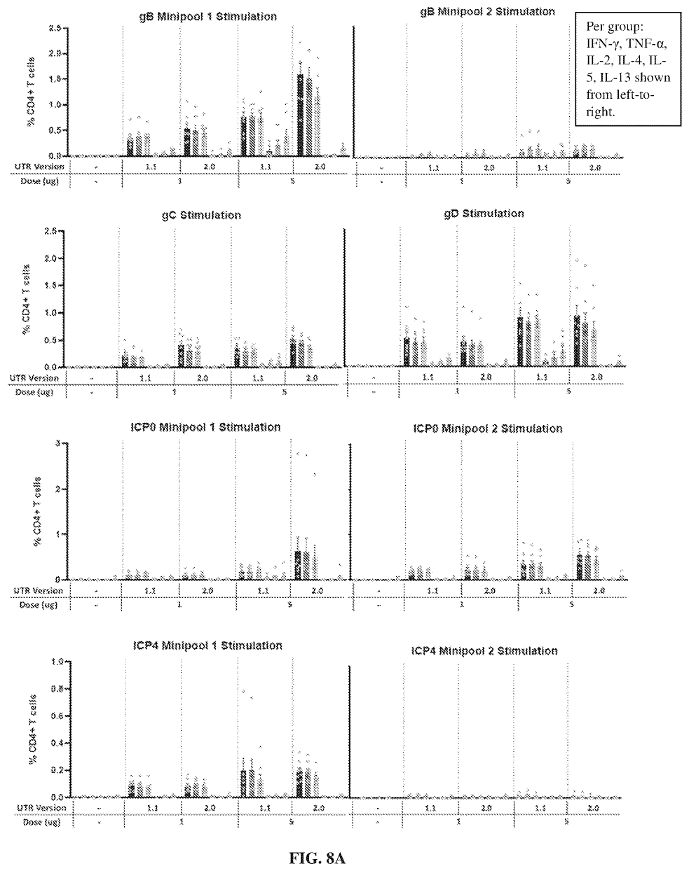

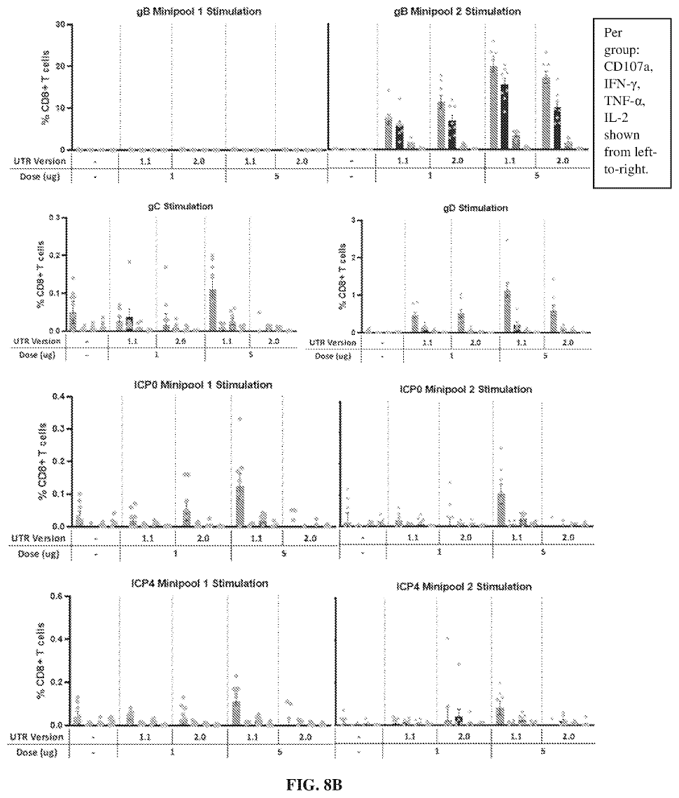

Robust CD4+ T cell responses to glycoprotein antigens were observed in all groups of vaccinated mice. Compared to v1.1 UTRs, use of v2.0 5′ and 3′ UTRs decreased the proportion of Th2 cells specific to peptides of gB, gC, and gD at both doses (FIG. 8A). Robust CD8+ T cell responses to gB were also observed, consistent with the presence of the immunodominant gB₄₉₈₋₅₀₅ peptide, as well as other HSV antigens (FIG. 8B). These results indicate that inclusion of a 5′ UTR of SEQ ID NO: 85, and 3′ UTRs of SEQ ID NOs: 88-92, in mRNAs further inhibits the generation of Th2 cells that are deleterious in HSV infections.

### Example 3: Immunization of Guinea Pigs with Varying Doses of mRNAs Encoding HSV Antigens During Latent HSV Infection

Guinea pigs are inoculated with HSV-2 to initiate a primary infection, with the virus establishing latency after the symptomatic phase resolves. The time course of this study is shown in FIG. 9. 14 days post-inoculation, guinea pigs are examined, and those with asymptomatic or severe primary disease (significant vaginal scarring) are excluded from the study. On days 21 and 35 post-inoculation, during the latent phase of infection, guinea pigs are administered an mRNA composition containing mRNAs encoding HSV antigens as described herein (50 μg mRNA total), or PBS as negative control.

From day 14 through day 21 post-inoculation, prior to immunization, and twice weekly after immunization, vaginal swabs are collected daily to quantify HSV-2 genome copies by qPCR and measure viral shedding. Starting from day 14 post-inoculation, guinea pigs are examined daily to check for genital lesions that indicate active HSV-2 replication. Sera are collected at day 21 (before the first vaccine dose), day 35 (2 weeks after the first vaccine dose but before the second dose), day 56 (3 weeks after the second dose), and day 70 (5 weeks after the second dose). At day 45 post-inoculation, 10 days after the second vaccine dose, blood is collected to analyze HSV-2-specific T cell responses in circulating T cells. At day 70, 5 weeks after the second vaccine dose, guinea pigs are euthanized, and spleens and vaginal mucosa are processed to analyze HSV-2-specific T cell responses. Dorsal root ganglia are also collected to analyze latent viral load.

### Example 4: Design of mRNA Encoding Truncated HSV Intracellular Antigens

The protein sequences of HSV proteins ICP0 and ICP4 were analyzed to identify regions containing high densities of T cell epitopes.

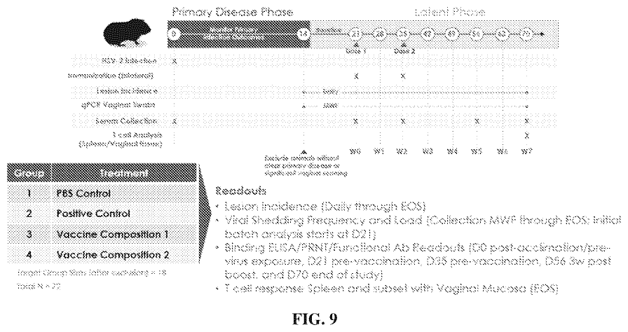

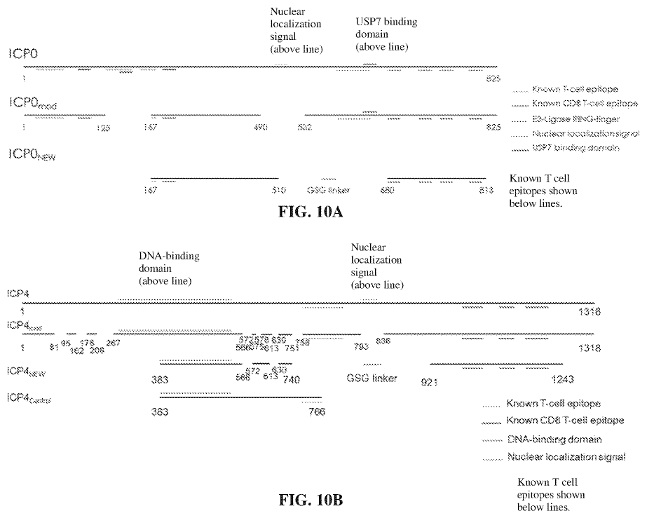

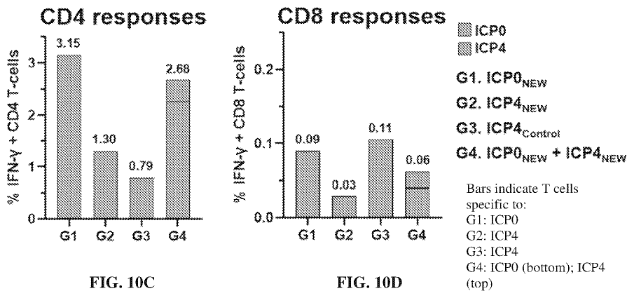

Regions of HSV ICP0 and HSV ICP4 that are rich in T cell epitopes are shown in FIGS. 10A-10B. Modified forms of each protein containing epitope-rich regions were designed.

These modified proteins contain higher densities of T cell epitopes than full-length forms and are thus useful for eliciting T cell responses to the proteins. Additionally, biologically active domains, such as the nuclear localization signal of HSV ICP0 and HSV ICP4, are absent from these modified forms, allowing proteins to be retained in the cytoplasm and more efficiently processed by the proteasome for presentation to T cells.

### Example 5: Immunization of Mice with Compositions Containing mRNAs Encoding Different HSV Intracellular Proteins

Mice are immunized with lipid nanoparticles containing the mRNAs encoding an HSV intracellular protein, ICP0 or ICP4, or both ICP0 and ICP4. ICP4 variants encoded by mRNAs include those set forth in SEQ ID NOs. 14 and 35, and control HSV ICP4 constructs for each of SEQ ID NOs. 14 and 35 that lack one or more known CD8+ T cell epitopes, as shown in FIG. 10B. The mRNAs and encoded antigens are shown in Table 13. A first dose (prime) is administered on day 0, and a second dose (booster) is administered on day 22. On day 28, mice are euthanized to collect spleens for analysis of immune cell populations, such as intracellular cytokine staining.

TABLE 7
Vaccine compositions containing mRNAs
encoding HSV intracellular proteins.

| | | mRNA | Sample | |
|---|---|---|---|---|
| | | dose | size | |
| | | (total | (# of | |
| Group | HSV antigens | mRNA) | mice) | SEQ ID NO: |
| 1 | None (PBS control) | 0 | 8 | None |
| 2 | ICP0 (variant) | 2 | 8 | 12 (mRNA) |
| | | | | 47 (protein) |
| 3 | ICP4 (variant) | 2 | 8 | 14 (mRNA) |
| | | | | 49 (protein) |
| 4 | Soluble ICP0 (variant) | 2 | 8 | 34 (mRNA) |
| | | | | 69 (protein) |
| 5 | Soluble ICP4 (variant) | 2 | 8 | 35 (mRNA) |
| | | | | 70 (protein) |
| 6 | ICP0 (variant) and | 2 | 8 | 12, 14 (mRNA) |
| | ICP4 (variant) | | | 47, 49 (protein) |
| 7 | ICP4 (control) | 2 | 8 | — |
| 8 | Soluble ICP4 (control) | 2 | 8 | — |

### Example 6: Immunization of Mice with Compositions Containing mRNAs Encoding Different HSV Intracellular Proteins

Mice were immunized with lipid nanoparticles containing the mRNAs encoding an HSV intracellular protein, ICP0 or ICP4, or both ICP0 and ICP4. ICP4 variants encoded by mRNAs included a variant ICP4 with the amino acid sequence set forth in SEQ ID NO: 49 and a control truncated ICP4 protein lacking several known T cell epitopes, as shown in FIG. 9B. A first dose (prime) containing 2 μg of mRNA per encoded protein was administered on day 0, and a second identical dose (booster) was administered on day 22. On day 28, mice were euthanized to collect spleens for analysis of immune cell populations by surface marker and intracellular cytokine staining. In all groups, administration of an mRNA encoding a given protein elicited IFN—γ+CD4+ to the encoded protein (FIG. 10C). In addition, mRNA encoding the ICP4 variant with the sequence set forth in SEQ ID NO: 49 elicited more IFN-γ+CD4+ T cells than mRNA encoding a truncated control ICP4. Some interference was observed when ICP4 and ICP0-encoding mRNAs were administered together, as fewer IFN-γ+CD4+ T cells specific to a given antigen were observed in mice administered both mRNAs (2 μg mRNA encoding ICP0 and 2 μg mRNA encoding ICP4), compared to mice administered mRNA encoding only one antigen (2 μg dose).

In contrast to the potent IFN-γ+CD4+ T cell response observed in this experiment, few CD8+ T cells specific to ICP0 or ICP4 were observed in each group (FIG. 10D). This result may arise from differences between the epitopes recognized by mouse CD8+ T cells or presented by mouse MHC-I proteins.

### Example 7: Evaluation of In Vitro Expression of Truncated HSV Glycoproteins

To evaluate expression of different HSV-2 gB variants, 10⁶ 293 cells were transfected with 500 ng of mRNA encoding (i) wild-type gB, (ii) gB with 120 amino acids deleted to truncate the cytoplasmic tail, (iii) gB with H506P and I507P mutations to stabilize the protein in prefusion conformation, or (iv) gB with both H506P and I507P mutations and a 120-amino acid deletion to truncate the cytoplasmic tail. Cells were then stained using a monoclonal antibodies specific to HSV-2 gB, or polyclonal mouse sera containing gB-specific antibodies, in combination with a fluorescently labeled secondary antibody, and flow cytometry was used to measure the extent of gB expression on the cell surface. Truncation of the cytoplasmic tail improved surface expression of gB by approximately two-fold (FIG. 11A). However, surface expression of gB containing H506P and I507P mutations was minimal.

In a similar experiment, 293 cells were transfected with mRNAs encoding wild-type gC, gC containing an F327A mutation, or gC containing an F327A mutation and a deletion of 11 C-terminal amino acids to truncate the cytoplasmic tail. 24 hours post-transfection, cells were stained with a monoclonal antibody specific to HSV-2 gC and a fluorescently labeled secondary antibody, and flow cytometry was used to measure the percentage of fluorescent cells (FIG. 11B), median fluorescence intensity (FIG. 11C). The percentage of fluorescent cells was multiplied by the MFI to calculate a measurement of total expression intensity (FIG. 11D). As with gB, truncation of the cytoplasmic tail markedly improved expression of gC. Additionally, the F327A mutation did not negatively impact expression. These results indicate that gCmutΔCT is robustly expressed by encoding mRNAs following mRNA introduction into cells.

### Example 8: Evaluation of Immune Sera for Inhibition of Human Complement-Binding by HSV gC

Sera from mice immunized as described in Examples 2-7 are collected at day 36, two weeks after administration of the second dose of an mRNA vaccine and tested for the ability to prevent gC from binding to human complement protein C3b in a competitive inhibition assay. In this assay, C3b is immobilized on a solid surface such as a 96-well plate, and biotinylated gC protein is pre-incubated with serum or a vehicle control to form immune complexes. Serum:gC immune complexes are then added to the C3b protein to assay the extent to which antibodies prevent C3b binding by gC. After washing to remove gC that is not bound to immobilized C3b, Streptavidin-HRP is added. The dilution of serum required to reduce the signal by 50%, relative to the signal observed with a vehicle control, is then calculated.

### Example 9: Immunization of Mice with mRNA Encoding HSV Proteins

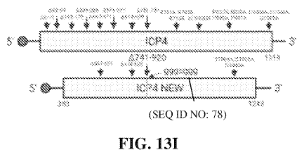

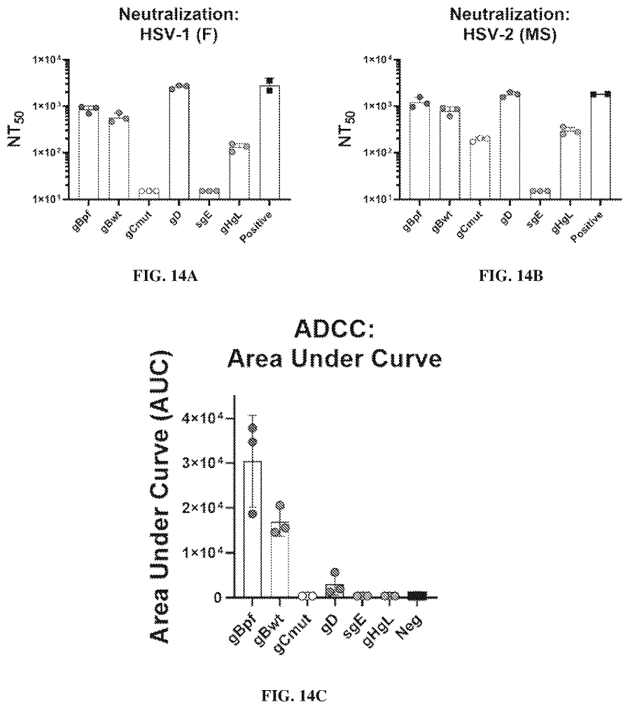

A panel of mRNA vaccine compositions, each containing lipid nanoparticles encapsulating an mRNA encoding a different HSV antigen, was tested in C57BL/6 mice. Mice were immunized with two doses of one of the compositions listed in Table 8, receiving the first dose on day 0 and the second dose on day 22. On day 36, two weeks after the second dose, serum was collected to evaluate neutralization activity against HSV infection in vitro and antibody-dependent cell-mediated cytotoxicity against HSV-1-infected cells. mRNA encoding prefusion or wild-type gB, gD, or gHgL proteins elicited antibodies that reduced infection of target cells by the F strain of HSV-1 or the MS strain of HSV-2 (FIGS. 14A-14B). Antibodies elicited by mRNA encoding gCmut showed neutralization activity against HSV-2, but not HSV-1, and antibodies elicited by mRNA encoding sgE did not neutralize either strain.

Sera from mice immunized with each mRNA composition shown in Table 8 were also tested for their ability to facilitate activation of effector cells in an ADCC assay. Sera from mice immunized with mRNA encoding either gB resulted in robust activation of the NFAT pathway in ADCC reporter cells, with the prefusion form (gBpf) eliciting greater activation than the wild-type form (FIG. 14C). mRNA encoding gD generated sera that resulted in minimal activation, while sera from mice immunized with other mRNAs encoding gCmut, sgE, or gHgL did not result in detectable activation. These results indicate that antibodies to gB on the surface of HSV-infected cells are essential for stimulating ADCC activity towards infected cells. Furthermore, the prefusion conformation of gB elicits more gB-specific antibodies than the wild-type conformation.

TABLE 8
Panel of mRNA vaccines tested in mice.

| | HSV antigen(s) | | |
|---|---|---|---|
| Group | encoded by mRNA | μg mRNA/antigen | SEQ ID NO: |
| 1 (N = 10) | gBpf | 5 | 2 (mRNA) |
| | | | 37 (protein) |
| 2 (N = 10) | gBwt | 5 | 1 (mRNA) |
| | | | 36 (protein) |
| 3 (N = 10) | gCmut | 5 | 3 (mRNA) |
| | | | 38 (protein) |
| 4 (N = 10) | gD | 5 | 4 (mRNA) |
| | | | 39 (protein) |
| 5 (N = 10) | Soluble gE (sgE or SgE) | 5 | 6 (mRNA) |
| | | | 41 (protein) |
| 6 (N = 10) | gHgL | 5 | 7, 8 (mRNA) |
| | | | 42, 43 (protein) |

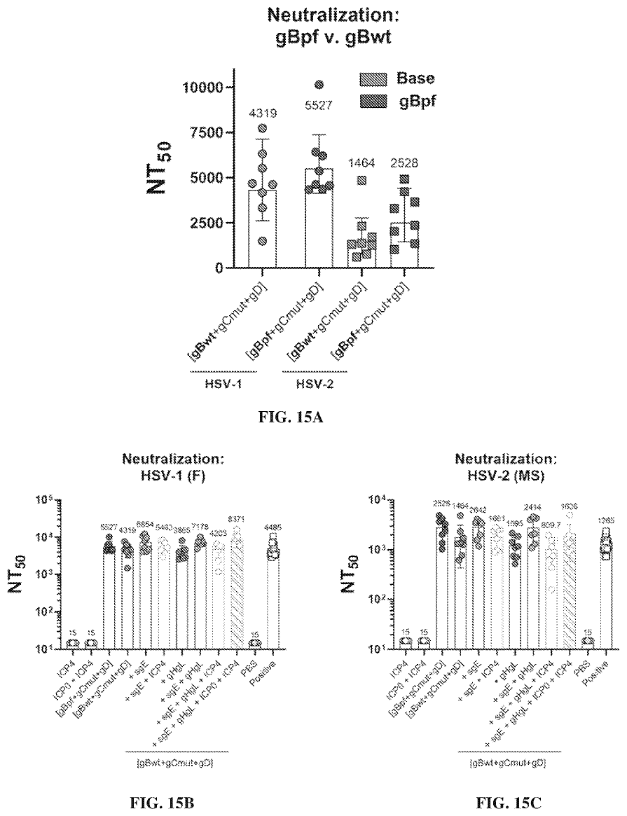

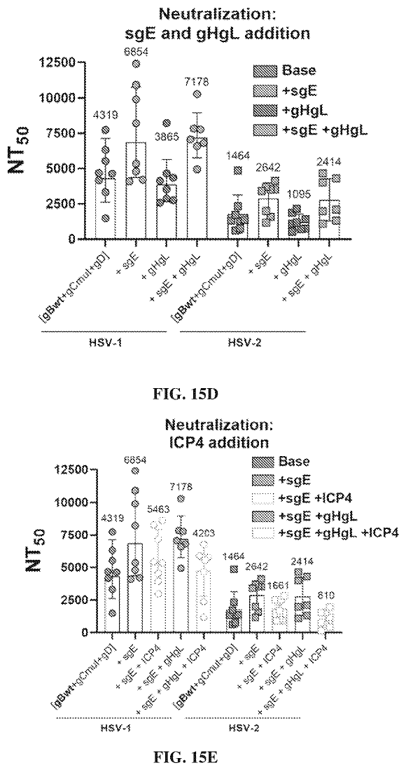

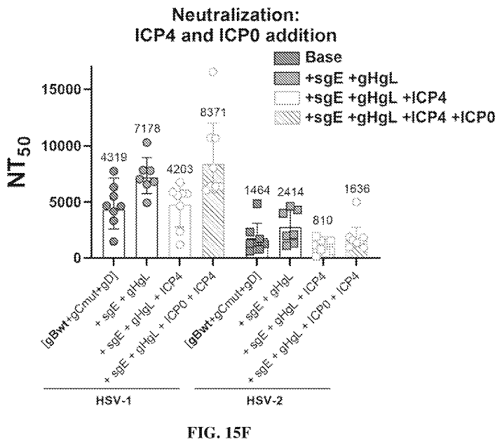

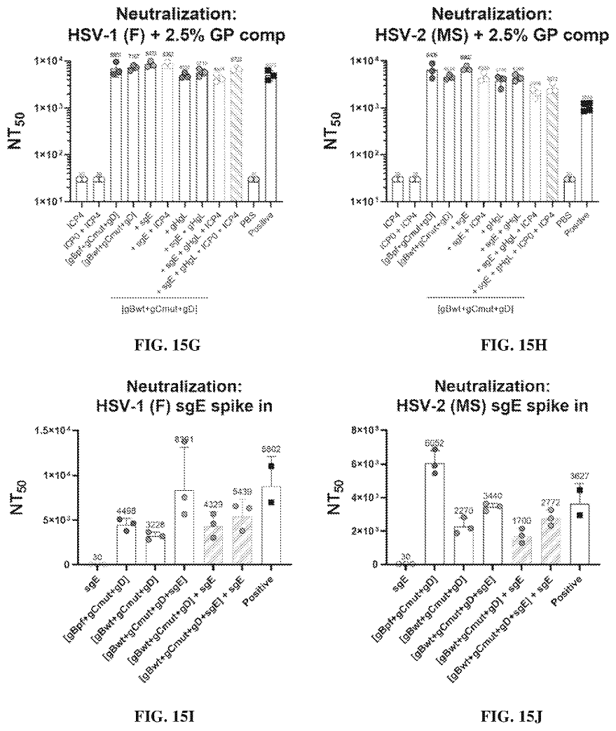

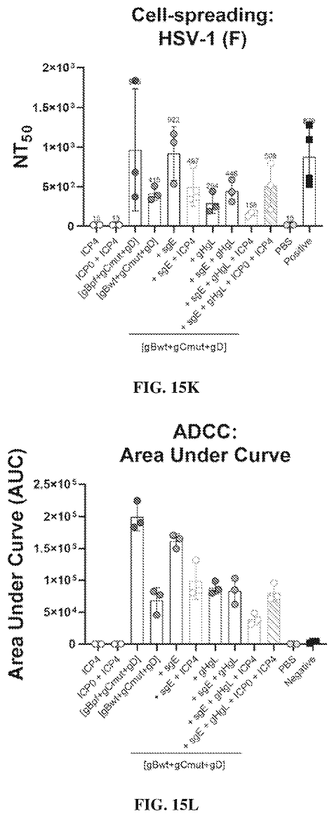

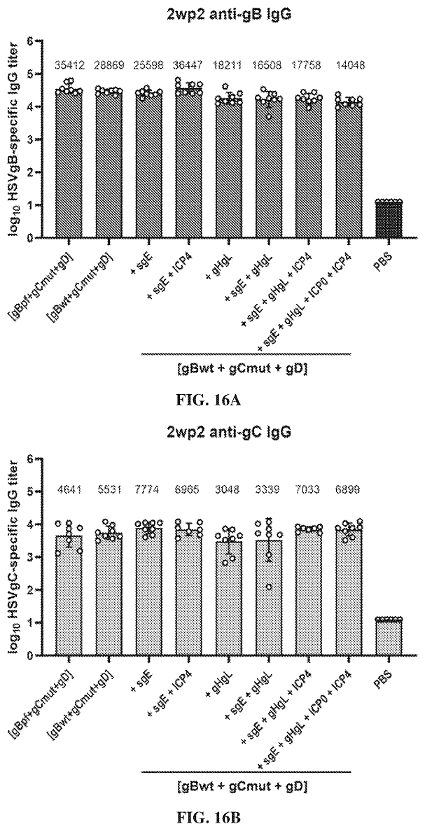

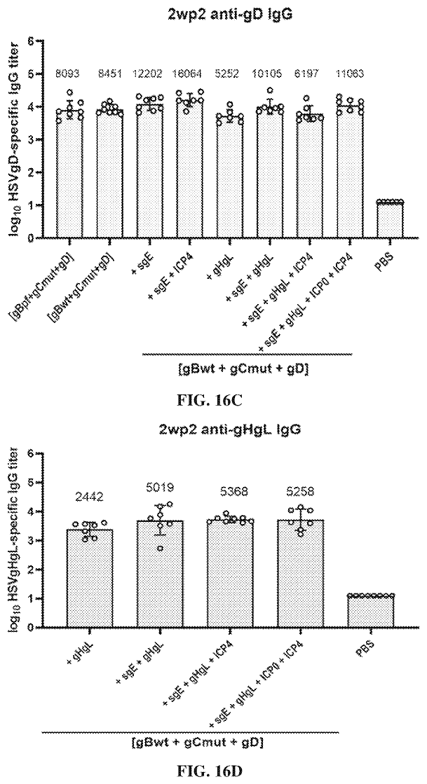

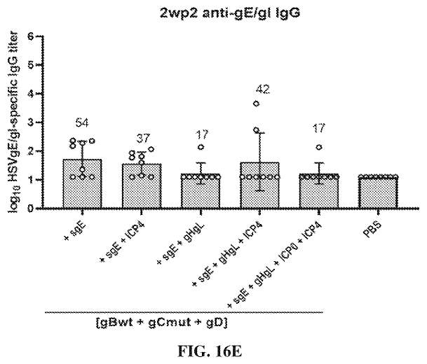

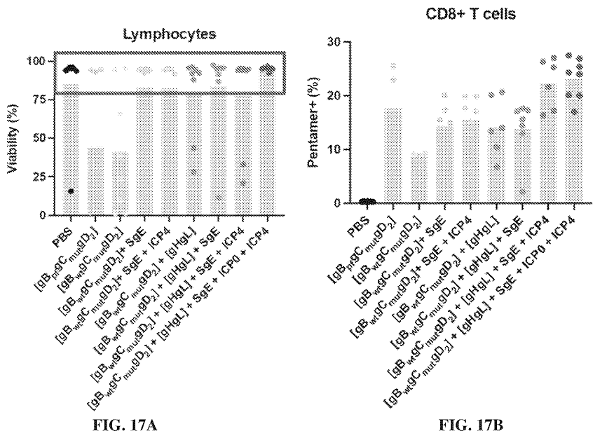

Another panel of mRNA vaccine compositions, each containing lipid nanoparticles encapsulating mRNAs encoding different HSV antigens, was tested in C57BL/6 mice using the same immunizations schedule described in the preceding paragraph. Mice were immunized with two doses of one of the compositions listed in Table 9, receiving the first dose on day 0 and the second dose on day 22. On day 36, two weeks after the second dose, serum was collected to evaluate neutralization activity against HSV infection in vitro (FIGS. 15A-15J), prevention of cell-cell spreading by replicating HSV-1 (FIG. 15K), antibody-dependent cell-mediated cytotoxicity against HSV-1-infected cells (FIG. 15L), and the amount of HSV surface protein-specific IgG (FIGS. 16A-16E).

TABLE 9
Panel of mRNA vaccines tested in mice.

| Group | HSV antigen(s) encoded by mRNA | μg mRNA/antigen | SEQ ID NO: |
|---|---|---|---|
| 1 (N = 8) | ICP4 | 2 | 13 (mRNA) |
| | | | 48 (protein) |
| 2 (N = 8) | ICP0, ICP4 | 2 | 11, 13 (mRNA) |
| | | | 46, 48 (protein) |
| 3 (N = 8) | gBpf, gCmut, gD | 2 | 2, 3, 4 (mRNA) |
| | | | 37, 38, 39 (protein) |
| 4 (N = 8) | gBwt, gCmut, gD | 2 | 1, 3, 4 (mRNA) |
| | | | 36, 38, 39 (protein) |
| 5 (N = 8) | gBwt, gCmut, gD, soluble gE | 2 | 1, 3, 4, 6 (mRNA) |
| | | | 36, 38, 39, 41 (protein) |
| 6 (N = 8) | gBwt, gCmut, gD, soluble gE, ICP4 | 2 | 1, 3, 4, 6, 13 (mRNA) |
| | | | 36, 38, 39, 41, 48 (protein) |
| 7 (N = 8) | gBwt, gCmut, gD, gHgL | 2 | 1, 3, 4, 7, 8 (mRNA) |
| | | | 36, 38, 39, 42, 43 (protein) |
| 8 (N = 8) | gBwt, gCmut, gD, gHgL, soluble gE | 2 | 1, 3, 4, 6, 7, 8 (mRNA) |
| | | | 36, 38, 39, 41, 42, 43 (protein) |
| 9 (N = 8) | gBwt, gCmut, gD, gHgL, soluble gE, | 2 | 1, 3, 4, 6, 7, 8, 13 (mRNA) |
| | ICP4 | | 36, 38, 39, 41, 42, 43, 48 (protein) |
| 10 (N = 8) | gBwt, gCmut, gD, gHgL, soluble gE, | 2 | 1, 3, 4, 6, 7, 8, 11, 13 (mRNA) |
| | ICP0, ICP4 | | 36, 38, 39, 41, 42, 43, 46, 48 (protein) |
| 11 (N = 8) | None (PBS control) | 0 | None |

With respect to mRNA encoding only gB, gC, and gD, sera from mice immunized with mRNA encoding the prefusion form of gB, rather than the wild-type form, were slightly more effective at neutralizing HSV-1 and HSV-2 (FIG. 15A). Sera from mice immunized with mRNA encoding gB, gC, and gD neutralized both the F strain of HSV-1 and MS strain of HSV-2, while sera from mice immunized with mRNA encoding only intracellular proteins (ICP4 and ICP0) did not neutralize either virus (FIGS. 15B-15C). The inclusion of mRNA encoding sgE elicited slightly higher neutralizing antibody titers towards both HSV-1 and HSV-2 (FIG. 15D). The addition of mRNA encoding HSV ICP4 to compositions including mRNAs encoding HSV gB, gC, gD, and sgE resulted in lower neutralization titers (FIG. 15E), while the further addition of mRNA encoding HSV ICP0 increased neutralization titers (FIG. 15F). To evaluate the role of complement in facilitating neutralization by antibodies in sera, some serum samples were supplemented with guinea pig complement to a final concentration of 2.5% (v/v) complement prior to neutralization assays. Addition of complement increased neutralization titers by about 10-fold relative to neutralization titers observed in unsupplemented sera (FIGS. 15G-15H). A similar experiment was performed to evaluate the role of sgE in neutralization of HSV-1 or HSV-2 by serum antibodies, in which sera were supplemented with sgE-specific sera prior to neutralization assays. The effect of sgE-specific sera supplementation was minimal in all assays, indicating that while the inclusion of an open reading frame encoding sgE in an mRNA slightly enhanced the generation of neutralizing antibodies to HSV-1 and HSV-2, the presence of sgE-specific antibodies is not a limiting factor in viral neutralization by serum antibodies (FIGS. 15I-15J).

Similar trends were observed with respect to the ability of sera to activate ADCC effector cells, with the prefusion conformation of gB, addition of sgE, and addition of HSV ICP0 increasing ADCC activity and inhibition of cell-cell spread, while the addition of HSV ICP4 reduced ADCC activity and inhibition of cell-cell spread (FIGS. 15K-15L). Sera from mice immunized with mRNA encoding gB, gC, and gD all contained IgG specific to each of these three antigens, regardless of whether the mRNA encoded the prefusion or wild-type gB, or the composition contained mRNA encoding additional HSV antigens (FIGS. 16A-16C). Each of the mRNAs encoding gHgL elicited robust antibodies specific to these proteins, as well (FIG. 16D). However, antibodies to gE and gI were largely absent from sera of mice immunized with mRNAs encoding sgE (FIG. 16E).

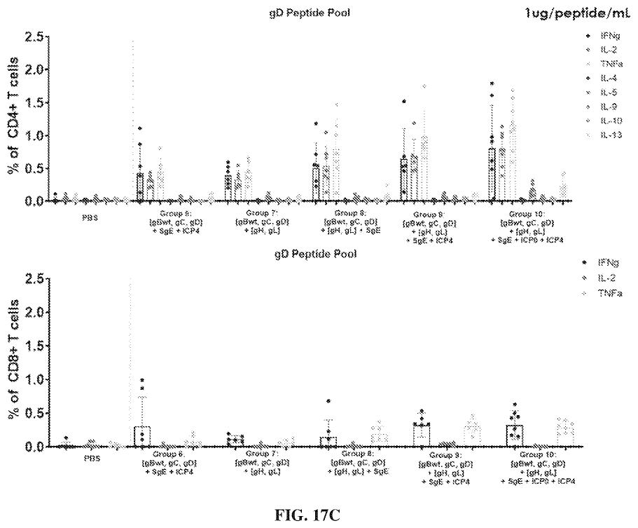

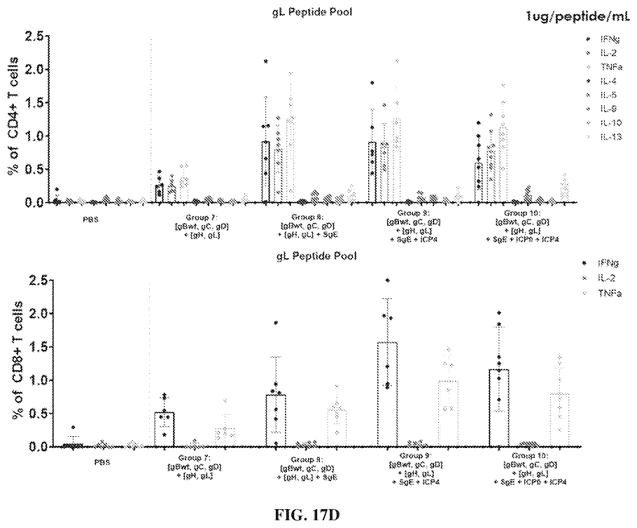

Mice were euthanized on day 36 post-first immunization, 2 weeks after receiving a second vaccine dose, to harvest spleens, which were processed for analysis of T cell responses by intracellular cytokine staining (ICS). Splenocytes were stimulated with one of a panel of peptide pools, each pool containing peptides from a single HSV antigen, in the presence of a Golgi blocker so that cells producing cytokines in response to stimulation would retain cytokines instead of secreting them. Cell surfaces were stained for lymphocyte markers, including CD3, CD4, and CD8, and cells were permeabilized and stained for multiple cytokines. Stained cells were incubated with a viability dye and analyzed by flow cytometry. The results of these experiments are shown in FIGS. 17A-17D.

Lymphocytes were largely viable, and not adversely affected by immunization or peptide stimulation (FIG. 17A). Among CD8+ T cells present in the spleen, the frequency of T cells that recognized a gB peptide was somewhat higher in mice that were immunized with mRNAs encoding more antigens than the baseline combination of gB, gC, and gD (FIG. 17B). Thus, the inclusion of additional antigens in the mRNA vaccines did not diminish the CD8+ T cell response to HSV gB.

Stimulation with a pool of peptides derived from gD revealed robust production of Th1-associated cytokines IFN-γ, IL-2, and TNF-α by CD4+ T cells, but lower production of these cytokines by CD8+ T cells (FIG. 17C). The inclusion of additional antigens, such as HSV sgE, ICP0, and ICP4 did not diminish these responses, though the inclusion of additional antigens resulted in the production of Th2-associated cytokines IL-5 and IL-13 by CD4+ T cells. Similar trends were observed in responses to the pool of gL peptides, except that robust production of Th1-associated cytokines was observed by both CD4+ T cells and CD8+ T cells (FIG. 17D).

Stimulation with pools of peptides from the N-terminal half of HSV ICP0 (ICP0-1) or the C-terminal half of HSV ICP0 (ICP0-2) resulted in some production of Th1-associated cytokines IFN-γ, IL-2, and TNF-α by CD4+ T cells, but negligible cytokine production by CD8+ T cells (data not shown). Stimulation with pools of peptides from the N-terminal half of HSV ICP4 (ICP4-1) resulted in some cytokine production, but lower cytokine production was observed with peptides from the C-terminal half of ICP4 (ICP4-2) (data not shown). Production of cytokines by CD8+ T cells was low after stimulation by either ICP4 peptide pool. In all cases, the inclusion of additional antigens, such as HSV sgE, gB, gC, gD, or gHgL, did not reduce the response to HSV ICP0 or HSV ICP4.

While T cell responses to peptides derived from HSV ICP0 and HSV ICP4 were marginal, mice do not always replicate observed T cell responses to the same antigens in humans. This discrepancy may be explained in part by differences in proteasome processing of antigens, MHC diversity effecting peptides presented to T cells, and differences in TCR repertoires between species. Thus, inclusion of mRNAs encoding HSV ICP0 and/or HSV ICP4 in mRNA vaccines may elicit protective T cell responses in humans.

### Example 10: Immunization of Mice with Varying Doses of mRNAs Encoding HSV Antigens

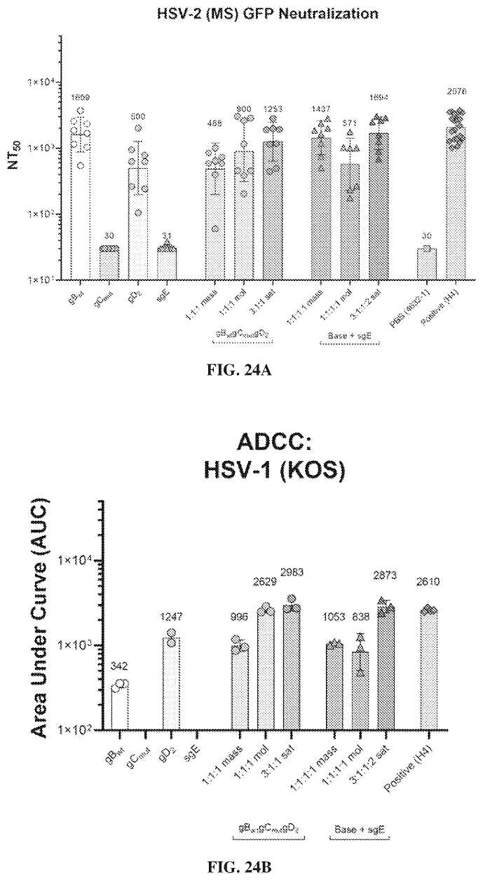

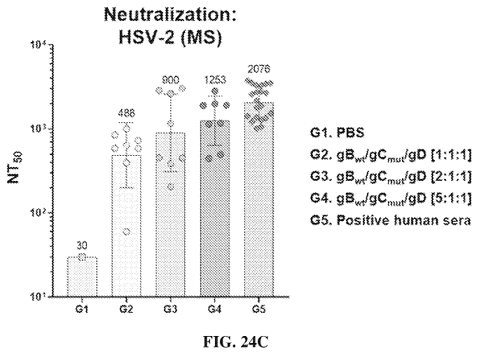

A panel of mRNA vaccine compositions, each containing lipid nanoparticles encapsulating mRNAs encoding different HSV antigens, was tested in C57BL/6 mice using the immunization schedule described in Methods above. Mice were immunized with two doses of one of the compositions listed in Table 10, receiving the first dose on day 0 and the second dose on day 22. In some groups, each LNP contained 2 μg mRNA encoding a given antigen (e.g., 2 μg mRNA encoding gB, 2 μg mRNA encoding gC, or 2 μg mRNA encoding gD), while in other groups, each LNP contained 0.4 μg mRNA encoding a given antigen (e.g., 0.4 μg mRNA encoding gB, 0.4 μg mRNA encoding gC, or 0.4 μg mRNA encoding gD). Control HSV ICP4 variants encoded by mRNAs administered to mice in Groups 8 and 16 lacked known CD8+ T cell epitopes that were present in HSV ICP4 variants encoded by the mRNA of SEQ ID NO: 14, as shown in FIG. 24B. Serum was collected on day 21, three weeks after the first dose, and day 36, two weeks after the second dose.

TABLE 10
Panel of mRNA vaccines tested in mice.

| Group | HSV antigen(s) encoded by mRNA | μg mRNA/antigen | SEQ ID NO: |
|---|---|---|---|
| 1 | None (PBS control) | 0 | |
| 2, 10 | gBwt, gCmut, gD | 2 (Group 2) or 0.4 (Group 10) | 1, 3, 4 (mRNA) |
| | | | 36, 38, 39 (protein) |
| 3, 11 | gBpf, gCmut, gD | 2 (Group 3) or 0.4 (Group 11) | 2, 3, 4 (mRNA) |
| | | | 37, 38, 39 (protein) |
| 4, 12 | gBpf, gCmut, gD, soluble gE | 2 (Group 4) or 0.4 (Group 12) | 2, 3, 4, 6 (mRNA) |
| | | | 37, 38, 39, 41 (protein) |
| 5, 13 | gBpf, gCmut, gD, soluble gE, ICP4 | 2 (Group 5) or 0.4 (Group 13) | 2, 3, 4, 6, 14 (mRNA) |
| | (variant) | | 37, 38, 39, 41, 49 (protein) |
| 6, 14 | gBpf, gCmut, gD, soluble gE, ICP0 | 2 (Group 6) or 0.4 (Group 14) | 2, 3, 4, 6, 12 (mRNA) |
| | (variant) | | 37, 38, 39, 41, 47 (protein) |
| 7, 15 | gBpf, gCmut, gD, soluble gE, ICP4 | 2 (Group 7) or 0.4 (Group 15) | 2, 3, 4, 6 (mRNA) |
| | (control) | | 37, 38, 39, 41 (protein) |
| 8, 16 | gBpf, gCmut, gD, soluble gE, gL | 2 (Group 8) or 0.4 (Group 16) | 2, 3, 4, 6, 8 (mRNA) |
| | | | 37, 38, 39, 41, 43 (protein) |
| 9, 17 | gBpf, gCmut, gD, soluble gE, gHgL | 2 (Group 9) or 0.4 (Group 17) | 2, 3, 4, 6, 7, 8 (mRNA) |
| | | | 37, 38, 39, 41, 42, 43 (protein) |

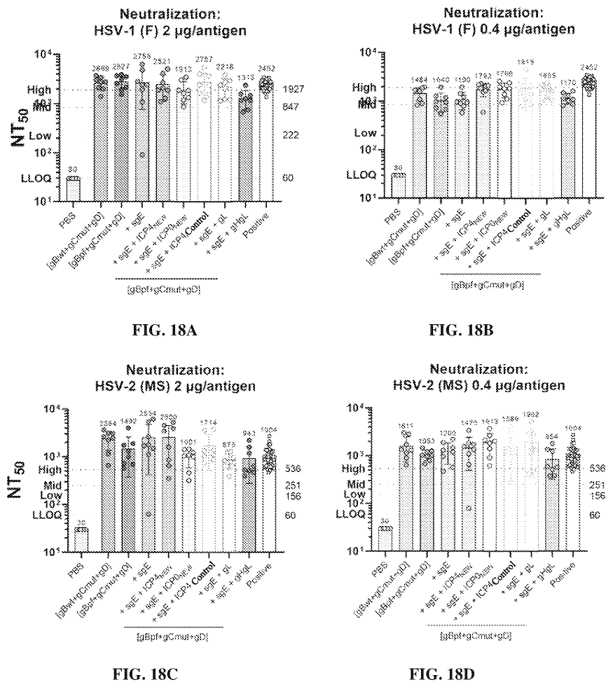

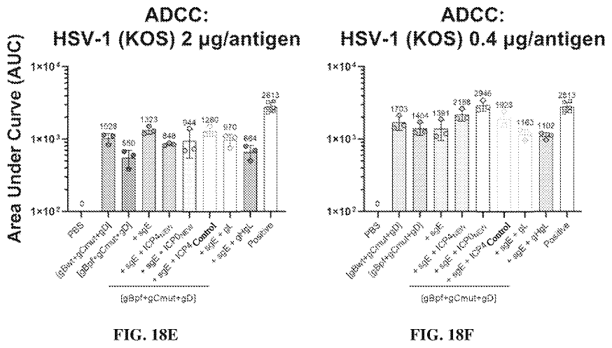

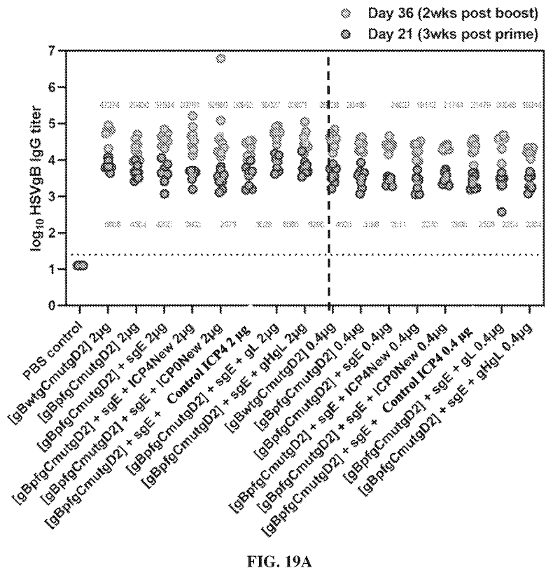

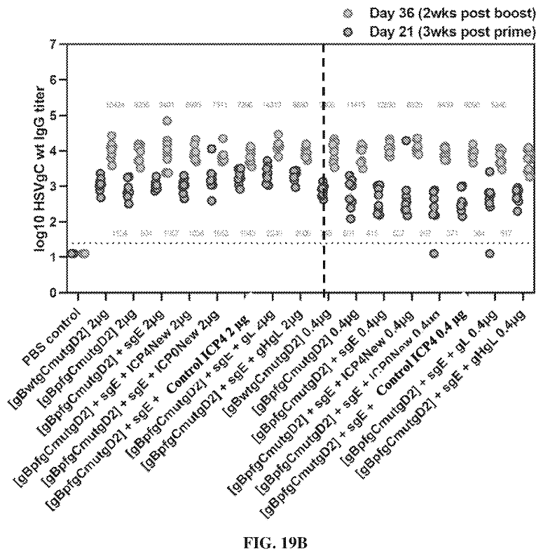

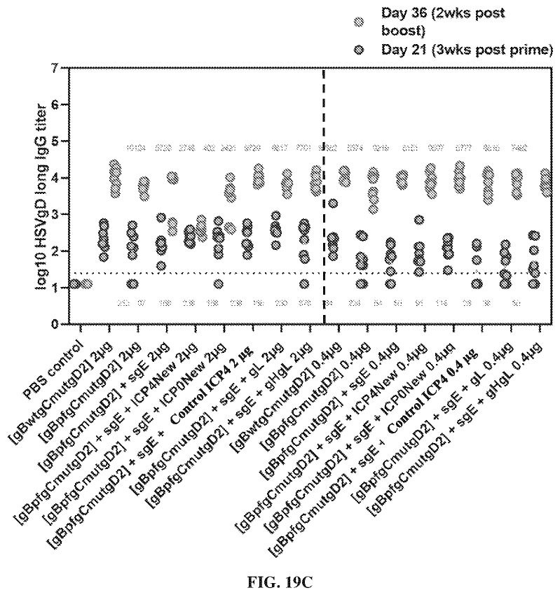

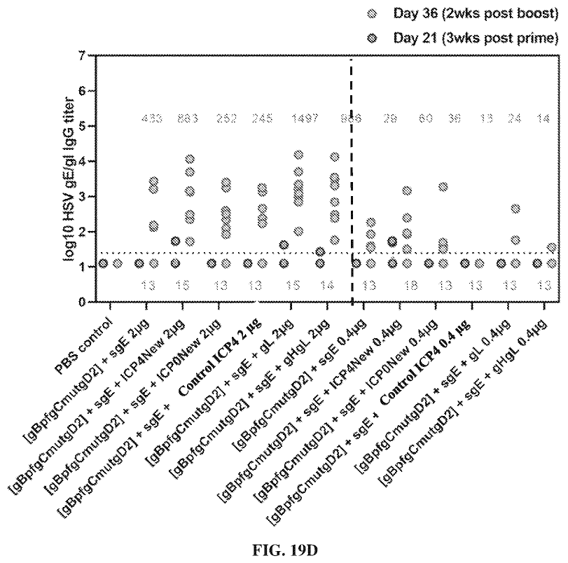

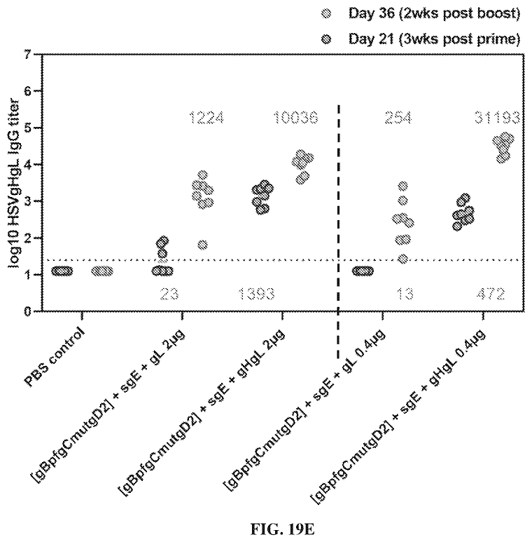

Sera from day 36 were tested for neutralization (FIGS. 18A-18D) and ADCC (FIGS. 18E-18F) activities. Sera from both timepoints were analyzed for amounts of HSV surface protein-specific IgG (FIGS. 19A-19E).

Neutralizing antibody titers from mice immunized with mRNA compositions were compared to neutralizing antibody titers in sera from a human serum repository. 80 human serum samples were rank ordered by neutralizing antibody titer towards a given HSV (HSV-1 F strain or HSV-2 MS strain) and divided into three groups of equal size. Within each, group, the geometric mean NT₅₀ was calculated and used as a benchmark for “high,” “medium,” or “low” neutralization activity. A potent serum sample was used as a positive control in neutralization assays. Reducing the dose of mRNA per encoded antigen by 5-fold, from 2 μg/antigen to 0.4 μg/antigen, caused only a modest decrease in neutralization titers, no more than 2.7-fold for HSV-1 (FIGS. 18A-18B) and no more than 2-fold for HSV-2 (FIGS. 18C-18D). However, reducing the dose improved ADCC activity, particularly for compositions including mRNA encoding HSV ICP0 (FIGS. 18E-18F). Higher doses, as well as particular routes of immunization, promote stronger germinal center reactions, skewing the antibody response towards immunodominant epitopes. See, e.g., Angeletti et al. Proc Nat Acad Sci USA. 2019. 116(27):13474-13479. Thus, lower mRNA doses may reduce this bias, favoring an immune response characterized by modestly lower neutralizing antibody titers but improved effector cell functions, such as antibody-dependent cell-mediated cytotoxicity, allowing for a balance between humoral and cellular immunity. This increased cellular immune response is particularly useful for treating or preventing herpesvirus infections by killing infected cells containing HSV proteins, before latent infection can be established.

Analysis of gB-, gC-, and gD-specific IgG titers by ELISA showed that a single dose containing 0.4 μg of mRNA per antigen elicited fewer antibodies towards the encoded proteins than a single dose containing 2 μg of the same mRNAs (FIGS. 19A-19C). However, in sera collected at day 36, two weeks after the second dose, antibodies were similar between both dose groups, with the exception of gD-specific titers, which were markedly lower in mice receiving 2 μg mRNA encoding HSV ICP4 (FIG. 19C). As seen in Example 9, titers of IgG specific to gE/gI were uniformly low (FIG. 19D). Finally, the addition of mRNA encoding gHgL to the composition, relative to the addition of mRNA encoding only gL, increased the titers of IgG specific to gHgL in both dose groups (FIG. 19E).

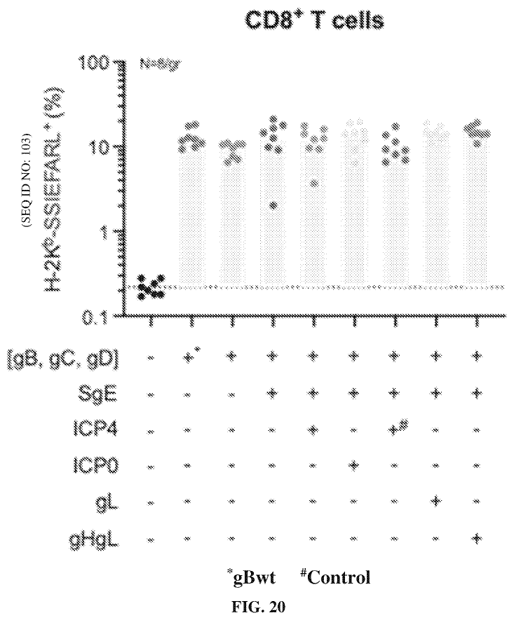

On day 36, mice were also euthanized to collect spleens for analysis of T cells as described in Methods above. In all groups, a substantial proportion of CD8⁺ T cells were specific to the SSIEFARL (SEQ ID NO: 103) epitope of HSV gB (FIG. 20). Robust Th1 responses towards HSV gD, gE, gL, and ICP0 were observed, as CD4+ T cells from mice immunized with mRNA compositions encoding those antigens produced Th1 cytokines IFN-γ, TNF-α, and IL-2 after stimulation with peptides from those antigens (Table 11). CD8+ cells also produced IFN-γ, TNF-α, and IL-2 after stimulation with peptides from gD, gE, and gL, but responded poorly to HSV ICP0 and ICP4 peptides (Table 11). Immunization with mRNAs encoding more antigens, such as the combination of gB, gC, gD, sgE, and ICP0, also elicited some Th2 cells, which produced IL-5, IL-9, IL-10, and IL-13 (data not shown). More CD4+ cells produced Th1 cytokines when stimulated with gD peptides than with peptides from other antigens, suggesting gD contains immunodominant epitopes (Table 11).

TABLE 11
T cell responses induced by vaccines shown in Table 6.

| | HSV antigen(s) encoded by mRNA | | | | | | | | | |
|---|---|---|---|---|---|---|---|---|---|---|
| | CD4+ Th1 cells | | | | | IFN-γ-producing CD8+ T cells | | | | |
| | gD | gE | gL | ICP0 | ICP4 | gD | gE | gL | ICP0 | ICP4 |
| None (PBS control) | | | | | | | | | | |
| gBwt, gCmut, gD | * | | | | | — | | | | |
| gBpf, gCmut, gD | * | | | | | — | | | | |
| gBpf, gCmut, gD, | * | — | | | | * | — | | | |
| soluble gE | | | | | | | | | | |
| gBwt, gCmut, gD, | * | — | | | — | — | — | | | — |
| soluble gE, ICP4 | | | | | | | | | | |
| (variant) | | | | | | | | | | |
| gBwt, gCmut, gD, | * | — | | ** | | ** | — | | — | |
| soluble gE, ICP0 | | | | | | | | | | |
| (variant) | | | | | | | | | | |
| gBwt, gCmut, gD, | * | — | | | — | — | — | | | — |
| soluble gE, ICP4 | | | | | | | | | | |
| (control) | | | | | | | | | | |
| gBwt, gCmut, gD, | * | — | — | | | — | — | * | | |
| soluble gE, gL | | | | | | | | | | |

— 0-0.25% of CD4+ or CD8+ cells
* 0.25-0.5% of CD4+ or CD8+ cells
** 0.5-1.0% of CD4+ or CD8+ cells
*** 1.0-1.5% of CD4+ or CD8+ cells

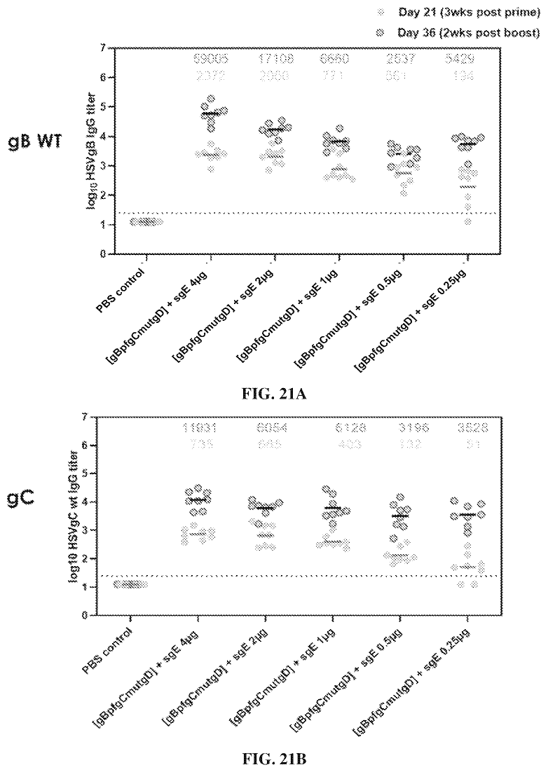

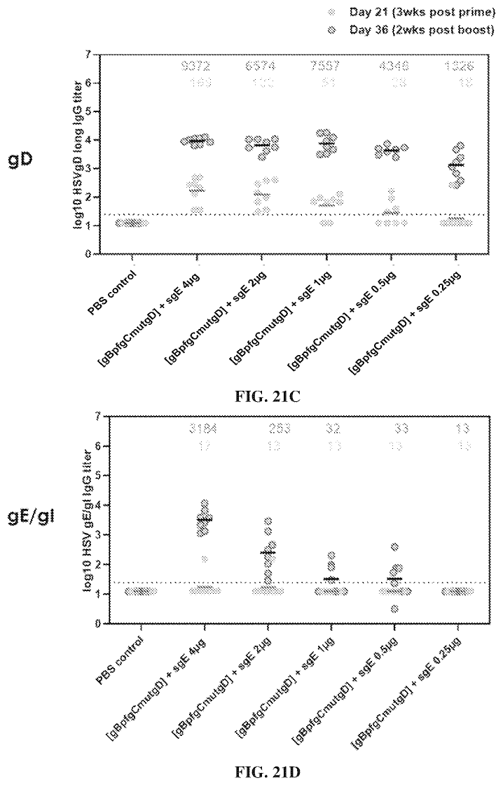

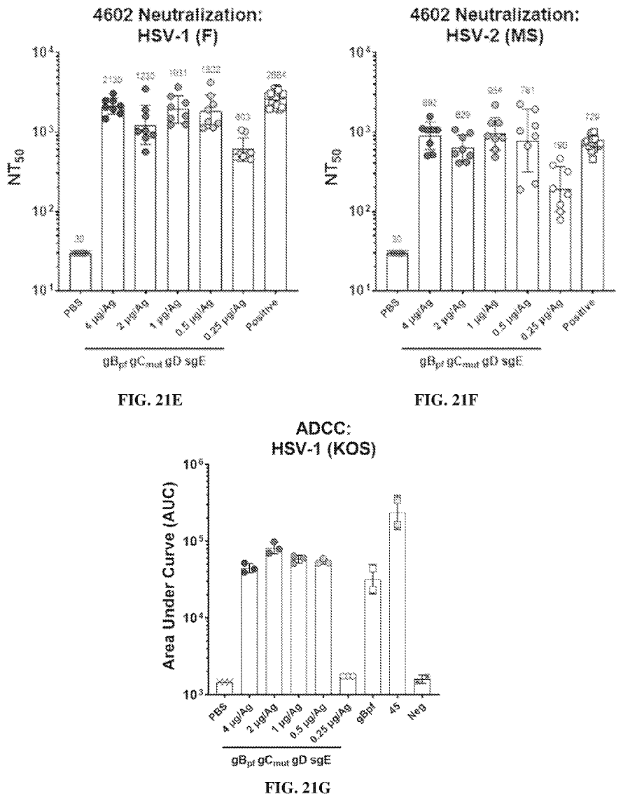

In a dose titration study, mice were immunized with lipid nanoparticles encapsulating different doses of the same mRNAs, encoding prefusion gB, complement-binding mutant gC, gD, and soluble gE. The doses of mRNA (μg per antigen) are shown in Table 12. Mice were immunized with two doses of one of the compositions listed in Table 12, receiving the first dose on day 0 and the second dose on day 22. Serum was collected on day 21, three weeks after the first dose, and day 36, two weeks after the second dose. Sera from both timepoints were analyzed for HSV protein-specific IgG (FIGS. 21A-21D), and sera from day 36 were tested for neutralization (FIGS. 21E-21F) and ADCC (FIG. 21G) activity.

Analysis of gB-, gC-, and gD-specific IgG titers by ELISA showed that lower doses of mRNA elicited lower IgG titers, for both day 21 and day 36 sera (FIGS. 21A-21C). IgG specific titers to gE/gI were negligible at day 21 in all groups, but increased after a second dose, with the highest titers being observed in mice that received the highest mRNA dose (FIG. 21D). With respect to neutralization activity, doses containing as little as 0.5 μg mRNA per antigen elicited neutralizing antibody titers that were comparable to those elicited by higher doses containing up to 4 μg mRNA per antigen (FIGS. 21E-21F). Similarly, ADCC activity was roughly equivalent in sera from mice immunized with compositions containing 4 μg, 2 μg, 1 μg, or 0.5 μg mRNA per antigen (FIG. 21G). These results indicate that relatively low amounts of mRNA are sufficient to elicit neutralizing antibodies to HSV, and antibodies that promote cellular effector functions towards HSV-infected cells.

TABLE 12
Doses of mRNA vaccines tested in mice.

| Group | HSV antigen(s) encoded by mRNA | μg mRNA/antigen | SEQ ID NO: |
|---|---|---|---|
| 1 | None (PBS control) | 0 | None |
| 2 | gBpf, gCmut, gD, soluble gE | 4 | 2, 3, 4, 6 (mRNA) |
| | | | 37, 38, 39, 41 (protein) |
| 3 | gBpf, gCmut, gD, soluble gE | 2 | 2, 3, 4, 6 (mRNA) |
| | | | 37, 38, 39, 41 (protein) |
| 4 | gBpf, gCmut, gD, soluble gE | 1 | 2, 3, 4, 6 (mRNA) |
| | | | 37, 38, 39, 41 (protein) |
| 5 | gBpf, gCmut, gD, soluble gE | 0.5 | 2, 3, 4, 6 (mRNA) |
| | | | 37, 38, 39, 41 (protein) |
| 6 | gBpf, gCmut, gD, soluble gE | 0.25 | 2, 3, 4, 6 (mRNA) |
| | | | 37, 38, 39, 41 (protein) |

### Example 11: Immunization of Mice with mRNA Encoding HSV Protein Variants

A panel of mRNA vaccine compositions, each containing lipid nanoparticles encapsulating mRNAs encoding different HSV antigens, was tested in C57BL/6 mice using the immunization schedule described in Methods above. Mice were immunized with two doses of one of the compositions listed in Table 13, receiving the first dose on day 0 and the second dose on day 22. One composition included mRNA encoding wild-type gB, another composition included mRNA encoding pre-fusion (pf) gB described in Methods above, and other compositions included mRNAs encoding different stabilized forms of gB. Each LNP contained 1 μg mRNA encoding a given antigen (e.g., 1 μg mRNA encoding gB, 1 μg mRNA encoding gC, and 1 μg mRNA encoding gD). Serum was collected on day 21, three weeks after the first dose, and day 36, two weeks after the second dose.

TABLE 13
Panel of mRNA vaccines tested in mice.

| | HSV antigen(s) | | | |
|---|---|---|---|---|
| Group | encoded by mRNA | gB mutations | μg mRNA/antigen | SEQ ID NO: |
| 1 (N = 8) | None (PBS control) | | 0 | None |
| 2 (N = 8) | gB_{wt}, gCmut, gD | | 1 | 1, 3, 4 (mRNA) |
| | | | | 36, 38, 39 (protein) |
| 3 (N = 8) | gB_{pf}, gCmut, gD | H510P | 1 | 2, 3, 4 (mRNA) |
| | | | | 37, 38, 39 (protein) |
| 4 (N = 8) | gB_{Δct}, gCmut, gD | Δ799-901 | 1 | 19, 3, 4 (mRNA) |
| | | | | 54, 38, 39 (protein) |
| 5 (N = 8) | gB_{2P#1}, gCmut, gD | I518P, A519P | 1 | 15, 3, 4 (mRNA) |
| | | | | 50, 38, 39 (protein) |
| 6 (N = 8) | gB_{2P#2}, gCmut, gD | M514P, L515P | 1 | 16, 3, 4 (mRNA) |
| | | | | 51, 38, 39 (protein) |
| 7 (N = 8) | gB_{2P#3}, gCmut, gD | H510P, V511P | 1 | 17, 3, 4 (mRNA) |
| | | | | 52, 38, 39 (protein) |
| 8 (N = 8) | gB_{2P#4}, gCmut, gD | H506P, I507P | 1 | 18, 3, 4 (mRNA) |
| | | | | 53, 38, 39 (protein) |
| 9 (N = 8) | gB_{2P#2Δct}, gCmut, gD | M514P, L515P, | 1 | 20, 3, 4 (mRNA) |
| | | Δ799-901 | | 55, 38, 39 (protein) |

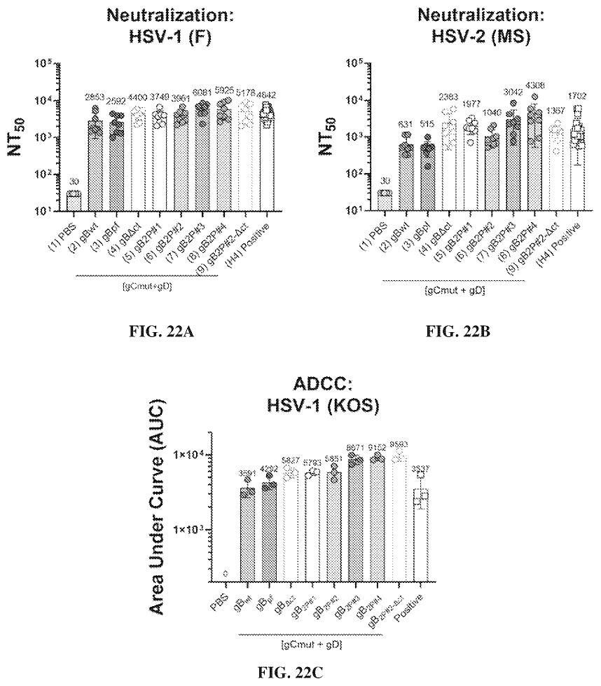

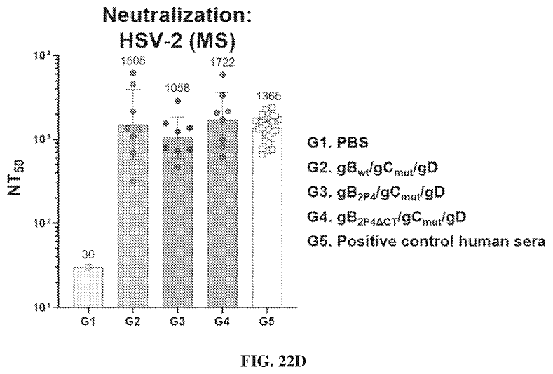

Sera from day 36 were tested for neutralization (FIGS. 22A-22B) and ADCC (FIG. 22C) activities. Relative to mRNAs encoding wild-type gB or H510P prefusion gB, mRNAs encoding gB proteins with two proline (2P) mutations to stabilize the gB in prefusion conformation, and/or deletions of amino acids 799-901 to remove the cytoplasmic tail, markedly increased neutralization titers. Neutralizing antibody titers against both HSV-1 and HSV-2 increased, but this effect was more pronounced for HSV-2. The greatest increases in neutralizing antibody titers were observed with the combination of H510P and V511P mutations, or with the combination of H506P and I507P mutations. Similarly, ADCC activity was higher in sera generated by immunization with compositions encoding gB lacking a cytoplasmic tail or containing 2P-stabilizing mutations.

In a follow-up experiment, mice were vaccinated using the same immunization and serum collection schedule described in the preceding two paragraphs, with lipid nanoparticles comprising mRNAs encoding HSV gCmut, gD, and either wild-type gB, gB containing 2P stabilizing mutations H506P and I507P, or the same 2P-stabilized gB with a deletion of 120 C-terminal amino acids (782-901) to remove the cytoplasmic tail. In contrast to the previous experiment, where lipid nanoparticles contained equal masses of mRNAs encoding each antigen, this experiment used a 2:1:1 ratio of mRNAs encoding gB:gC:gD. At this ratio, the combination with mRNA encoding 2P-stabilized gB elicited similar neutralizing antibody titers to the combination with mRNA-encoding wild-type gB. However, removal of the cytoplasmic tail of gB still improved immunogenicity relative to a matched control with a full-length cytoplasmic tail (FIG. 22D).

Another panel of mRNA vaccine compositions, each containing lipid nanoparticles encapsulating mRNAs encoding different HSV antigens, was tested in C57BL/6 mice using the immunization schedule described in Methods above. Mice were immunized with two doses of one of the compositions listed in Table 14, receiving the first dose on day 0 and the second dose on day 22. Each composition included mRNA encoding prefusion H510 gB, gC, gD, and gE as described in Example 11, as well as variants of gL or gHgL. Each LNP contained 2 μg mRNA encoding a given antigen (e.g., 2 μg mRNA encoding gB, 2 μg mRNA encoding gC, and 2 g mRNA encoding gD). Some compositions included wild-type gL, while one composition included mRNA encoding a variant gL lacking a signal peptide (Δsp) to prevent secretion, and another composition contained an mRNA encoding gH and gLjoined by a linker. One composition included equal masses of an mRNA encoding gH and mRNA encoding gL (1 μg each), while another composition included equimolar amounts of the two mRNAs (2 μg total mRNA encoding gH and gL). Serum was collected on day 21, three weeks after the first dose, and day 36, two weeks after the second dose.

TABLE 14
Panel of mRNA vaccines tested in mice.

| | HSV antigen(s) | Subcellular | | |
|---|---|---|---|---|
| Group | encoded by mRNA | localization | μg mRNA/antigen | SEQ ID NO: |
| 1 (N = 8) | None (PBS control) | | 0 | None |
| 2 (N = 8) | gB_{pf}, gCmut, gD, sgE, gL | Secreted gL | 2 | 2, 3, 4, 6, 8 (mRNA) |
| | | | | 37, 38, 39, 41, 43 (protein) |
| 3 (N = 8) | gB_{pf}, gCmut, gD, sgE, gLAsp | Cytosolic gL | 2 | 2, 3, 4, 6, 9 (mRNA) |
| | | | | 37, 38, 39, 41, 44 (protein) |
| 4 (N = 8) | gB_{pf}, gCmut, gD, sgE, | Membrane gL and | 2 | 2, 3, 4, 6, 7, 8 (mRNA) |
| | gHgL(equal mass gH mRNA | secreted gH | | 37, 38, 39, 41, 42, 43 (protein) |
| | and gL mRNA) | | | |
| 5 (N = 8) | gB_{pf}, gCmut, gD, sgE, gHgL | Membrane gL and | 2 | 2, 3, 4, 6, 7, 8 (mRNA) |
| | (equimolar gH mRNA and gL | secreted gH | | 37, 38, 39, 41, 42, 43 (protein) |
| | mRNA) | | | |
| 6 (N = 8) | gB_{pf}, gCmut, gD, sgE, gHgL | Membrane gHgL | 2 | 2, 3, 4, 6, 10 (mRNA) |
| | (linked gHgL) | | | 37, 38, 39, 41, 45 (protein) |

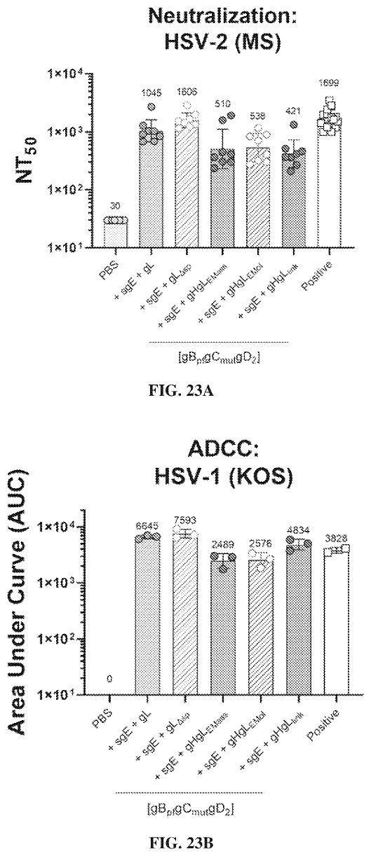

Sera from day 36 were tested for neutralization (FIG. 23A) and ADCC (FIG. 23B) activities. Addition of mRNA encoding gH to the compositions, either as a separate protein from gL or as a protein linked to gL, reduced both neutralizing antibody titers and ADCC activity.

On day 36, mice were also euthanized to collect spleens for analysis of T cells as described in Methods above. For assaying responses to gB, two pools containing distinct peptides from gB, were prepared, and cells were stimulated separately with each pool. The same approach was used to assay responses to gH. The results of these stimulations are shown in Table 15.

TABLE 15
T cell responses induced by vaccines shown in Table 10.

| | HSV antigen(s) encoded by mRNA | | | | | | | | | | | | | |
|---|---|---|---|---|---|---|---|---|---|---|---|---|---|---|
| | CD4+ Th1 cells | | | | | | | IFN-γ-producing CD8+ T cells | | | | | | |
| | gB | gB | | | gH | gH | | | gB | gB | | | gH | gH |
| | pool | pool | | | pool | pool | | gB | pool | pool | | | pool | pool |
| | 1 | 2 | gD | gE | 1 | 2 | gL | 1 | 1 | 2 | gD | gE | 1 | 2 |
| None (PBS control) | | | | | | | | | | | | | | |
| gB_{pf}, gCmut, | * | — | ** | — | | | * | — | **** | * | — | | | ** |
| gD, sgE, gL | | | | | | | | | | | | | | |
| gB_{pf}, gCmut, | ** | — | *** | — | | | * | — | **** | ** | — | | | *** |
| gD, sgE, gL_{Δsp} | | | | | | | | | | | | | | |
| gB_{pf}, gCmut, | * | — | * | — | * | ** | * | — | **** | * | — | — | — | ** |
| gD, sgE, gHgL | | | | | | | | | | | | | | |
| (equal mass | | | | | | | | | | | | | | |
| gH mRNA and | | | | | | | | | | | | | | |
| gL mRNA) | | | | | | | | | | | | | | |
| gB_{pf}, gCmut, | * | — | * | — | * | ** | * | — | **** | — | — | — | — | * |
| gD, sgE, gHgL | | | | | | | | | | | | | | |
| (equimolar gH | | | | | | | | | | | | | | |
| mRNA and gL | | | | | | | | | | | | | | |
| mRNA) | | | | | | | | | | | | | | |
| gB_{pf}, gCmut, | * | — | ** | — | —/* | ** | * | — | **** | — | — | — | — | * |
| gD, sgE, gHgL | | | | | | | | | | | | | | |
| (linked gHgL) | | | | | | | | | | | | | | |

— 0-0.25% of CD4+ or CD8+ cells
* 0.25-0.5% of CD4+ or CD8+ cells
** 0.5-1.0% of CD4+ or CD8+ cells
*** 1.0-1.5% of CD4+ or CD8+ cells
**** >1.5% of CD4+ or CD8+ cells

Robust Th1 responses towards gB (pool 1), gD, gH (pools 1 and 2), and gL were observed, as CD4+ T cells from mice immunized with mRNA compositions encoding those antigens produced Th1 cytokines IFN-γ, TNF-αL, and IL-2 after stimulation with peptides from those antigens. CD8+ cells also produced IFN-γ, TNF-αL, and IL-2 after stimulation with peptides from gB (pool 2), gD, and gL. Th2 cells responses were minimal, as few CD4+ T cells produced IL-4, IL-5, or IL-13 (data not shown). More CD4+ cells produced Th1 cytokines when stimulated with gD peptides than with peptides from other antigens, suggesting gD contains immunodominant MHC-JJ epitopes (Table 15). The same bias towards gB peptides occurred in CD8+ T cells, suggesting gB contains immunodominant MHC-J epitopes.

Consistent with the reduction in antibody titers, adding mRNA encoding gH to the compositions reduced the levels of gB and gD-specific Th1 cells, as well as the levels of gD-specific CD8+ T cells.

With respect to gL-specific responses, compositions containing mRNA encoding gL, gLΔsp, or equal masses of mRNAs encoding gH and gL elicited similar levels of gL-specific Th1 and CD8+ T cells. However, compositions containing equimolar amounts of mRNAs encoding gH and gL, or mRNA encoding the linked gHgL, elicited lower CD8+ T cell responses to gL.

With respect to gH-specific responses, compositions with either equimolar amounts or equal masses of mRNAs encoding separate gH and gL proteins elicited equivalent Th1 and CD8+ T cell responses. However, mRNAs encoding the linked gHgL elicited fewer gH-specific Th1 and CD8+ T cells.

### Example 12: Immunization of Mice with Compositions Containing Different Ratios of mRNAs Encoding HSV Antigens

Mice were immunized with lipid nanoparticles containing the mRNAs encoding one or more HSV antigens. The ratios of mRNAs encoding each antigen are shown in Table 16. Some compositions contained equal masses of each mRNA, while others contained equimolar amounts of mRNAs. In some compositions, mRNA ratios were varied so that the doses of mRNAs administered were saturating with respect to antibody titers generated. Saturating doses were determined using data from the dose de-escalation study shown in Example 11. A first dose (prime) was administered on day 0, and a second dose (booster) was administered on day 22. Serum was collected on day 21, three weeks after the administration after the first dose but before booster dose administration, and on day 36, two weeks after the administration of the booster dose. Sera were evaluated for antiviral activities such as neutralization and ADCC activity.

TABLE 16
Vaccine compositions containing varying ratios of mRNAs encoding HSV antigens.

| | HSV antigen(s) | | | |
|---|---|---|---|---|
| Group | encoded by mRNA | mRNA Ratio | mRNA dose (μg) | SEQ ID NO: |
| 1 (N = 8) | gB_{wt} | N/A | 1 | 1 (mRNA) |
| | | | | 36 (protein) |
| 2 (N = 8) | gCₘᵤₜ (F327A) | N/A | 1 | 3 (mRNA) |
| | | | | 38 (protein) |
| 3 (N = 8) | gD | N/A | 1 | 4 (mRNA) |
| | | | | 39 (protein) |
| 4 (N = 8) | Soluble gE (sgE) | N/A | 1 | 6 (mRNA) |
| | | | | 41 (protein) |
| 5 (N = 8) | gB_{wt}, gCₘᵤₜ, gD | 1:1:1 mass | 1 | 1, 3, 4 (mRNA) |
| | | | (0.33 each) | 36, 38, 39 (protein) |
| 6 (N = 8) | gB_{wt}, gCₘᵤₜ, gD | 1:1:1 molarity | 1 | 1, 3, 4 (mRNA) |
| | | | (0.49, 0.28, 0.23) | 36, 38, 39 (protein) |
| 7 (N = 8) | gB_{wt}, gCₘᵤₜ, gD | 3:1:1 saturating | 1 | 1, 3, 4 (mRNA) |
| | | | (0.74, 0.14, 0.12) | 36, 38, 39 (protein) |
| 8 (N = 8) | gB_{wt}, gCₘᵤₜ, gD, sgE | 1:1:1:1 mass | 1 | 1, 3, 4, 6 (mRNA) |
| | | | (0.25 each) | 36, 38, 39, 41 (protein) |
| 9 (N = 8) | gB_{wt}, gCₘᵤₜ, gD, sgE | 1:1:1:1 molarity | 1 | 1, 3, 4, 6 (mRNA) |
| | | | (0.39, 0.22, 0.19, 0.20) | 36, 38, 39, 41 (protein) |
| 10 (N = 8) | gB_{wt}, gCₘᵤₜ, gD, sgE | 3:1:1:2 saturating | 1 | 1, 3, 4, 6 (mRNA) |
| | | | (0.59, 0.11, 0.09, 0.21) | 36, 38, 39, 41 (protein) |

mRNAs encoding either gB or gD elicited neutralizing antibodies that effectively reduced infection of target cells by the MS strain of HSV-2 (FIG. 24A). Trivalent compositions containing mRNAs encoding gB, gC, and gD, and quadrivalent compositions containing mRNAs encoding gB, gC, gD, and sgE also elicited neutralizing antibodies, with titers that varied between those elicited by gD-encoding mRNA compositions and those elicited by gB-encoding mRNA compositions. In mice immunized with trivalent and quadrivalent compositions, antibody titers increased as the amount of gB-encoding mRNA in the composition increased relative to other mRNAs.

Sera from mice immunized with each mRNA composition shown in Table 16 were also tested for their ability to facilitate activation of effector cells in an antibody-dependent cellular cytotoxicity (ADCC) assay. Sera from mice immunized with mRNA encoding either gB or gD resulted in robust activation of the NFAT pathway in ADCC reporter cells, with gD-encoding mRNA eliciting a stronger ADCC response (FIG. 24B). As with neutralizing antibody responses, trivalent and quadrivalent compositions containing more gB-encoding mRNA elicited stronger ADCC responses. Unexpectedly, sera from mice immunized with trivalent and quadrivalent compositions promoted greater NFAT activation in ADCC reporter cells than sera from mice immunized with monovalent compositions encoding only a single antigen. This was unexpected in part because monovalent and multivalent compositions elicited similar NT₅₀ titers when tested for a neutralizing antibody response. These results indicate that antibodies to gB and gD on the surface of HSV-infected cells contribute to ADCC activity that removes infected cells, and that a combined response to multiple surface-expressed antigens, including gB, gD, and gC, or gB, gD, gC, and gE, facilitates greater antiviral ADCC activity than a response to a single antigen.

In a follow-up experiment, mice were vaccinated using the same immunization and serum collection schedule described in the preceding three paragraphs, with lipid nanoparticles comprising mRNAs encoding HSV gB, gCmut, and gD. Lipid nanoparticles contained mass ratios of 1:1:1, 2:1:1, or 5:1:1 of mRNAs encoding gB, gCmut, and gD with a total of 1 μg mRNA in each lipid nanoparticle (e.g., 0.5 μg mRNA encoding gB, 0.25 μg mRNA encoding gCmut, or 0.25 μg mRNA encoding gD for the 2:1:1 group). Increasing the amount of mRNA encoding gB also increased the neutralizing antibody titers obtained by immunization, with the most potent neutralizing antibody response occurring in mice vaccinated with composition containing a 5:1:1 ratio of mRNAs encoding gB, gCmut, and gD (FIG. 24C).

### Example 13: Immunization of Mice with Compositions Containing Combinations of mRNAs Encoding HSV Glycoproteins and Intracellular Proteins

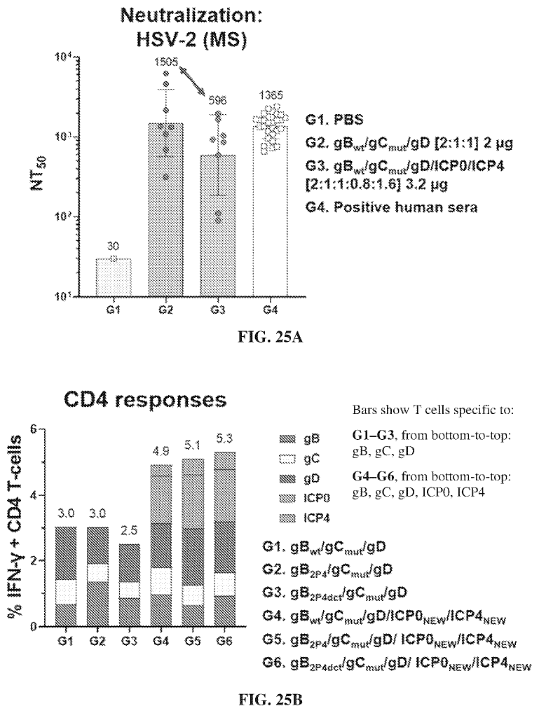

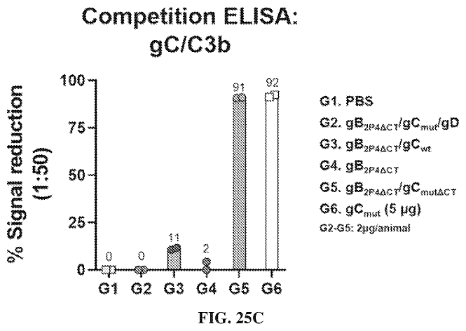

A panel of mRNA vaccine compositions, each containing lipid nanoparticles encapsulating mRNAs encoding different HSV antigens, was tested in mice using the same immunizations schedule described in Methods above. The antigens encoded by mRNAs of each composition, and the ratios of mRNAs in each composition, are shown in Table 17. In compositions containing mRNAs encoding gB, gC, and gD, the mRNAs were present in a mass ratio of 2 gB:1 gC: 1 gD. In compositions containing mRNAs encoding gB, gC, gD, ICP0, and ICP4, the mRNAs were present in a mass ratio of 2 gB:1 gC:1 gD:2.8 CP0:1.6 ICP4. Mice were immunized with lipid nanoparticles containing mRNAs, receiving the first dose on day 0 and the second dose on day 22. Serum was collected on day 21, three weeks after the first dose, and day 36, two weeks after the second dose. FIGS. 25A and 25C show the results of neutralization and ADCC assays using day 36 sera. Mice were also euthanized on day 36 to collect spleens for analysis of T cells by surface marker and intracellular cytokine staining, as shown in FIG. 25B.

TABLE 17
Panel of mRNA vaccines tested in mice.

| | | mRNA dose | Sample size | |
|---|---|---|---|---|
| Group | HSV antigens | (μg mRNA per antigen) | (# of mice) | SEQ ID NO: |
| 1 | None (PBS) | — | 8 | — |
| 2 | gBwt, gCmut, gD | 4 (2, 1, 1) | 8 | 1, 3, 4 (mRNA) |
| | | | | 36, 38, 39 (protein) |
| 3 | gB2P4, gCmut, gD | 4 (2, 1, 1) | 8 | 18, 3, 4 (mRNA) |
| | | | | 53, 38, 39 (protein) |
| 4 | gB2P4ΔCT, gCmut, gD | 4 (2, 1, 1) | 8 | 21, 3, 4 (mRNA) |
| | | | | 46, 38, 39 (protein) |
| 5 | gB2P4ΔCT, gCwt, gD | 4 (2, 1, 1) | 8 | 21, 30, 4 (mRNA) |
| | | | | 56, 65, 39 (protein) |
| 6 | gB2P4ΔCT, gD | 3 (2, 1) | 8 | 21, 4 (mRNA) |
| | | | | 56, 39 (protein) |
| 7 | gB2P4ΔCT, gCmutΔCT, gD | 4 (2, 1, 1) | 8 | 21, 28, 4 (mRNA) |
| | | | | 56, 63, 39 (protein) |
| 8 | gB2P4ΔCT, gCACT, gD | 4 (2, 1, 1) | 8 | 21, 27, 4 (mRNA) |
| | | | | 56, 62, 39 (protein) |
| 9 | gBwt, gCmut, gD, ICP0 | 5.4 (2, 1, 1, 0.8, 0.6) | 8 | 1, 3, 4, 12, 14 (mRNA) |
| | (variant), ICP4 (variant) | | | 36, 38, 39, 47, 49 (protein) |
| 10 | gB2P4, gCmut, gD, ICP0 | 5.4 (2, 1, 1, 0.8, 0.6) | 8 | 18, 3, 4, 12, 14 (mRNA) |
| | (variant), ICP4 (variant) | | | 53, 38, 39, 47, 49 (protein) |
| 11 | gB2P4ΔCT, gCmut, gD, | 5.4 (2, 1, 1, 0.8, 0.6) | 8 | 21, 3, 4, 12, 14 (mRNA) |
| | ICP0 (variant), ICP4 | | | 56, 38, 39, 47, 49 (protein) |
| | (variant) | | | |

Consistent with the results shown in FIG. 24C, mice immunized with mRNAs encoding gB, gC, and gD exhibited robust neutralizing antibody titers. However, some interference was observed when mRNAs encoding ICP0 and ICP4, which are targeted primarily by T cells, were included in the composition, as neutralizing antibody titers were lower in those mice (FIG. 25A). Despite this interference with the generation of neutralizing antibodies, the inclusion of mRNAs encoding ICP0 and ICP4 markedly increased the total number of IFN-γ+CD4+ T cells specific to HSV-2 antigens (FIG. 25B). These results indicate that HSV glycoproteins B, C, and D induce a strong CD4+ T cell responses.

In the same study, some mice were immunized with compositions containing mRNAs encoding different forms of gC, including wild-type gC, gC containing an F327A substitution (gCmut), or gC containing an F327A substitution and deletion of 11 amino acids to truncate the cytoplasmic tail (gCmutΔCT). Sera collected at day 36 post-immunization were used in an gC:C3b binding inhibition assay described in Example 8, to determine the extent to which the compositions elicited gC-specific antibodies capable of blocking C3b binding, which contributes to immune evasion by HSV-2. Immunization with mRNAs encoding gCmutΔCT increased the antibody response to gC approximately 100-fold, relative to immunization with mRNA encoding wild-type gC (FIG. 25C). Together with the in vitro expression data of Example 1, showing improved expression of gCmutΔCT compared to wild-type gC, these results indicate that truncation of the cytoplasmic tail of gC improves both protein expression and immunogenicity.

In another experiment, mice were immunized according to the same immunization, serum collection, and T cell collection schedule, with another panel of compositions, shown in Table 18. Lipid nanoparticles contained a total of 5.2 μg mRNA, with the ratios of mRNAs encoding gB, gC containing an F327A substitution, gD, ICP0, and ICP4 being 2 gB:1 gC:1 gD:0.4 ICP0:0.8 ICP4. One composition contained mRNA encoding wild-type gB, while the other contained mRNA encoding gB containing H506P and I507P substitutions and a deletion of amino acids 782-901 to truncate the cytoplasmic tail (gB2P4ΔCT).

TABLE 18
Panel of mRNA vaccines tested in mice.

| | | mRNA dose | Sample size | |
|---|---|---|---|---|
| Group | HSV antigens | (μg mRNA per antigen) | (# of mice) | SEQ ID NO: |
| 1 | None (PBS) | — | 8 | — |
| 2 | ICP0 (variant) | 2 | 8 | 12 (mRNA) |
| | | | | 47 (protein) |
| 3 | ICP4 (variant) | 2 | 8 | 14 (mRNA) |
| | | | | 49 (protein) |
| 4 | ICP0 (variant), ICP4 | 4 | 8 | 12, 14 (mRNA) |
| | (variant) | (2, 2) | | 47, 49 (protein) |
| 5 | gCmut, gD, ICP0 | 2.4 | 8 | 3, 4, 12 (mRNA) |
| | (variant) | (1, 1, 0.4) | | 38, 39, 47 (protein) |
| 6 | gCmut, gD, ICP4 | 2.4 | 8 | 3, 4, 14 (mRNA) |
| | (variant) | (1, 1, 0.8) | | 38, 39, 49 (protein) |
| 7 | gCmut, gD, ICP0 | 3.2 | 8 | 3, 4, 12, 14 (mRNA) |
| | (variant), ICP4 (variant) | (1. 1, 0.4, 0.8) | | 38, 39, 47, 49 (protein) |
| 8 | gBwt, gCmut, gD | 2 | 8 | 1, 3, 4 (mRNA) |
| | | (1, 0.5, 0.5) | | 36, 38, 39 (protein) |
| 9 | gB2P4ΔCT, gCmut, gD | 2 | 8 | 21, 3, 4 (mRNA) |
| | | (1, 0.5, 0.5) | | 56, 38, 39 (protein) |
| 10 | gB2P4ΔCT, gCmut, gD | 1.5 | 8 | 21, 3, 4 (mRNA) |
| | | (0.5, 0.5, 0.5) | | 56, 38, 39 (protein) |
| 11 | gBwt, gCmut, gD, ICP4 | 4.8 | 8 | 1, 3, 4, 14 (mRNA) |
| | (variant) | (2, 1, 1, 0.8) | | 36, 38, 39, 49 (protein) |
| 12 | gB2P4ΔCT, gCmut, | 4.8 | 8 | 21, 3, 4, 14 (mRNA) |
| | gD, ICP4 (variant) | (2, 1, 1, 0.8) | | 56, 38, 39, 49 (protein) |
| 13 | gB2P4ΔCT, gCmut, | 3.4 | 8 | 21, 3, 4, 14 (mRNA) |
| | gD, ICP4 (variant) | (1, 1, 1, 0.4) | | 56, 38, 39, 49 (protein) |
| 14 | gBwt, gCmut, gD, ICP0 | 5.2 | 8 | 1, 3, 4, 12, 14 (mRNA) |
| | (variant), ICP4 (variant) | (2, 1, 1, 0.4, 0.8) | | 36, 38, 39, 47, 49 (protein) |
| 15 | gB2P4ΔCT, gCmut, | 5.2 | 8 | 21, 3, 4, 12, 14 (mRNA) |
| | gD, ICP0 (variant), | (2, 1, 1, 0.4, 0.8) | | 56, 38, 39, 47, 49 (protein) |
| | ICP4 (variant) | | | |
| 16 | gB2P4ΔCT, gCmut, | 3.8 | 8 | 21, 3, 4, 12, 14 (mRNA) |
| | gD, ICP0 (variant), | (1, 1, 1, 0.4, 0.4) | | 56, 38, 39, 47, 49 (protein) |
| | ICP4 (variant) | | | |

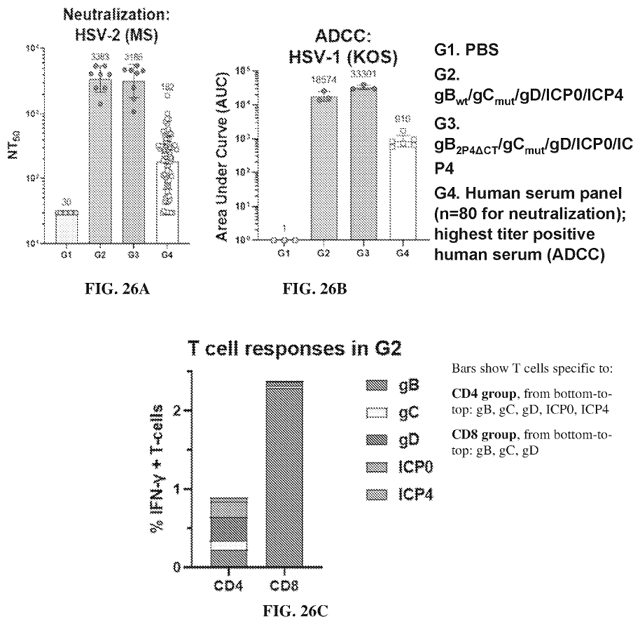

Both compositions elicited robust neutralizing antibody and ADCC responses, with ADCC activity being slightly higher in mice immunized with the composition containing mRNA encoding gB2P4ΔCT (FIGS. 26A-26B). Additionally, IFN-γ+CD4+ T cells specific to each encoded antigen were detected in mice (FIG. 26C). The CD8+ T cell response, while also high, was predominated by T cells specific to a peptide of gB that is immunodominant in mice.

### Example 14: Immunization of Mice with Compositions Containing mRNAs Encoding Different gE and gI Proteins

Mice are immunized with lipid nanoparticles containing the mRNAs encoding gB, gC, gD, and optionally gE and/or gI. The antigens encoded by the mRNAs of each composition are shown in Table 19. A first dose (prime) is administered on day 0, and a second dose (booster) is administered on day 22. Serum is collected on day 21, three weeks after the administration after the first dose but before booster dose administration, and on day 36, two weeks after the administration of the booster dose. Sera are evaluated for antiviral activities such as neutralization, ADCC activity, and prevention of cell-cell spread by HSV.

TABLE 19
Vaccine compositions containing mRNAs encoding different HSV gE and gI proteins.

| | | mRNA dose | Sample size | |
|---|---|---|---|---|
| Group | HSV antigens | (μg mRNA per antigen) | (# of mice) | SEQ ID NO: |
| 1 | gBwt, gCmut, gD | 0.5 | 8 | 1, 3, 4 (mRNA) |
| | | | | 36, 38, 39 (protein) |
| 2 | gBwt, gCmut, gD | 0.5, 0.5, 0.75 | 8 | 1, 3, 4 (mRNA) |
| | | | | 36, 38, 39 (protein) |
| 3 | gBwt, gCmut, gD, | 0.5 | 8 | 1, 3, 4, 6 (mRNA) |
| | soluble gE | | | 36, 38, 39, 41 (protein) |
| 4 | gBwt, gCmut, gD, | 0.5 | 8 | 1, 3, 4, 6, 24 (mRNA) |
| | soluble gE, | | | 36, 38, 39, 41, 59 (protein) |
| | soluble gI | | | |
| 5 | gBwt, gCmut, gD, | 0.5 | 8 | 1, 3, 4, 5 (mRNA) |
| | gE | | | 36, 38, 39, 40 (protein) |
| 6 | gBwt, gCmut, gD, | 0.5 | 8 | 1, 3, 4, 5, 23 (mRNA) |
| | gE, gI | | | 36, 38, 39, 40, 58 (protein) |
| 7 | gBwt, gCmut, gD, | 0.5 | 8 | 1, 3, 4, 25 (mRNA) |
| | gEACT | | | 36, 38, 39, 60 (protein) |
| 8 | gBwt, gCmut, gD, | 0.5 | 8 | 1, 3, 4, 25, 26 (mRNA) |
| | gEACT, gIACT | | | 36, 38, 39, 50, 51 (protein) |

“ACT” indicates a deletion of the cytoplasmic tail.

### Example 15: Immunization of Mice with Compositions Containing mRNAs Encoding Different gD Proteins

Mice are immunized with lipid nanoparticles containing the mRNAs encoding gB, gC, gD, and optionally gE and/or gI. The antigens encoded by the mRNAs of each composition are shown in Table 20. gD-gp120 refers to fusion protein of HSV gD and HIV-1 gp120. A first dose (prime) is administered on day 0, and a second dose (booster) is administered on day 22. Serum is collected on day 21, three weeks after the administration after the first dose but before booster dose administration, and on day 36, two weeks after the administration of the booster dose. Sera are evaluated for antiviral activities such as neutralization, ADCC activity, and prevention of cell-cell spread by HSV.

TABLE 20
Vaccine compositions containing mRNAs encoding different HSV gD proteins.

| | | mRNA dose | Sample size | |
|---|---|---|---|---|
| Group | HSV antigens | (μg mRNA per antigen) | (# of mice) | SEQ ID NO: |
| 1 | gBpf, gCmut, gD | 1 | 8 | 2, 3, 4 (mRNA) |
| | | | | 37, 38, 39 (protein) |
| 2 | gBpf, gCmut, soluble | 1 | 8 | 2, 3, 31 (mRNA) |
| | gD | | | 37, 38, 66 (protein) |
| 3 | gBpf, gCmut, gD- | 1 | 8 | 2, 3, 32 (mRNA) |
| | gp120 | | | 37, 38, 67 (protein) |
| 4 | gBpf, gCmut, soluble | 1 | 8 | 2, 3, 33 (mRNA) |
| | gD-gp120 | | | 37, 38, 68 (protein) |

SEQUENCES

| | | SEQ ID |
|---|---|---|
| Name | Sequence | NO: |
| HSV gB ACT | | |
| SEQ ID NO: 104 consists of from 5′ end to 3′ end: 5′ UTR SEQ ID NO: 85, mRNA ORF | | |
| SEQ ID NO: 19, and 3′ UTR SEQ ID NO: 88. | | |
| Cap | C1 | |
| 5′ UTR | GAGGAAAUCGCAAAAUUUGCUCUUCGCGUUAGAUUUCUUUUAGUUUUCUC | 85 |
| | GCAACUAGCAAGCUUUUUGUUCUCGCC | |
| ORF of mRNA Construct | AUGCGGGGUGGCGGACUGGUGUGCGCCCUGGUGGUCGGUGCACUUGUGGC | 19 |
| (excluding the stop codon) | CGCUGUGGCUAGCGCUGCACCAGCCGCACCCAGAGCCAGCGGAGGUGUGG | |
| | CCGCGACCGUUGCAGCCAACGGAGGCCCGGCUUCUCAGCCUCCUCCUGUG | |
| | CCCAGCCCCGCUACCACCAAGGCCCGUAAGCGGAAGACCAAGAAGCCUCC | |
| | CAAGCGGCCCGAGGCUACACCACCACCCGACGCCAACGCAACUGUUGCUG | |
| | CUGGCCACGCCACCCUGAGGGCCCACCUGCGGGAGAUCAAGGUGGAGAAC | |
| | GCCGACGCCCAGUUCUACGUGUGUCCACCUCCAACCGGCGCCACCGUGGU | |
| | ACAGUUCGAGCAACCUCGCCGGUGCCCCACUCGACCCGAGGGCCAGAACU | |
| | ACACCGAGGGCAUCGCCGUGGUGUUCAAGGAGAACAUCGCCCCAUACAAG | |
| | UUCAAGGCCACCAUGUACUACAAGGACGUGACCGUGAGCCAGGUGUGGUU | |
| | CGGCCACCGGUACAGCCAGUUCAUGGGCAUCUUCGAGGAUCGGGCCCCAG | |
| | UGCCCUUCGAGGAGGUGAUCGACAAGAUCAACGCCAAGGGCGUGUGCCGG | |
| | AGCACCGCCAAGUACGUGCGGAACAACAUGGAGACCACCGCCUUCCACCG | |
| | GGACGACCACGAGACCGACAUGGAGCUGAAGCCCGCCAAGGUGGCCACAC | |
| | GGACAAGCCGGGGCUGGCACACCACCGACCUGAAGUACAACCCAAGCCGC | |
| | GUGGAGGCGUUCCACCGGUACGGCACCACCGUGAACUGCAUCGUGGAGGA | |
| | GGUGGACGCCCGGAGCGUGUACCCCUACGACGAAUUCGUGCUGGCCACCG | |
| | GCGACUUCGUGUACAUGAGCCCCUUCUACGGCUACCGGGAGGGCAGCCAC | |
| | ACCGAGCACACCAGCUACGCCGCCGACCGGUUCAAGCAGGUGGACGGCUU | |
| | CUACGCCCGGGACCUGACCACUAAAGCCCGGGCCACCUCACCAACCACCC | |
| | GGAACCUGCUGACCACCCCAAAGUUCACCGUGGCCUGGGACUGGGUGCCC | |
| | AAGCGACCCGCCGUGUGCACCAUGACCAAGUGGCAGGAAGUGGACGAGAU | |
| | GCUGCGGGCCGAAUACGGCGGCAGCUUCCGGUUCAGCAGCGACGCCAUCA | |
| | GCACCACCUUCACCACCAACCUGACCCAGUACAGCCUGAGCCGGGUGGAU | |
| | CUGGGCGACUGCAUCGGCAGAGACGCUCGCGAGGCCAUCGACCGGAUGUU | |
| | CGCCCGGAAGUACAACGCCACCCACAUCAAGGUGGGCCAGCCCCAGUACU | |
| | ACCUCGCCACCGGCGGCUUCCUGAUCGCCUACCAGCCCUUGCUGAGCAAC | |
| | ACCCUGGCCGAGCUGUACGUGCGGGAGUACAUGCGGGAGCAGGACCGGAA | |
| | GCCCCGGAACGCCACACCCGCCCCUCUGAGAGAAGCCCCUUCCGCCAACG | |
| | CCAGCGUGGAGCGGAUCAAGACCACCAGCAGCAUCGAGUUCGCCCGGCUG | |
| | CAGUUCACCUACAACCACAUCCAGCGGCACGUGAACGACAUGCUGGGUCG | |
| | GAUCGCCGUGGCUUGGUGCGAGCUGCAGAACCACGAGCUGACCCUGUGGA | |
| | ACGAGGCCCGGAAGCUGAACCCCAACGCCAUCGCCAGCGCUACUGUGGGC | |
| | CGGAGGGUAAGCGCUCGGAUGCUGGGCGACGUGAUGGCCGUGAGCACGUG | |
| | UGUGCCCGUGGCCCCAGACAACGUGAUCGUGCAGAACAGCAUGCGGGUGA | |
| | GUAGCCGGCCUGGAACCUGCUACAGCCGGCCCCUGGUGUCUUUCCGGUAC | |
| | GAGGACCAGGGUCCCCUGAUCGAGGGCCAGCUGGGCGAGAACAACGAGCU | |
| | GCGGCUGACUCGCGACGCUCUGGAGCCCUGCACCGUGGGCCACAGACGGU | |
| | ACUUCAUCUUCGGCGGCGGCUACGUGUACUUCGAGGAGUACGCCUACAGC | |
| | CACCAGCUGAGCCGGGCCGACGUGACCACCGUGAGCACCUUCAUCGACCU | |
| | GAACAUCACCAUGCUGGAGGACCACGAGUUCGUGCCCCUGGAGGUGUACA | |
| | CCCGGCACGAGAUCAAGGACAGCGGCCUGCUGGAUUACACCGAGGUGCAG | |
| | CGGCGGAACCAGCUGCACGACCUGCGGUUCGCCGACAUCGACACCGUGAU | |
| | ACGGGCCGACGCAAACGCCGCCAUGUUCGCCGGCCUGUGCGCCUUCUUCG | |
| | AGGGCAUGGGCGACCUGGGCAGAGCCGUGGGCAAGGUGGUGAUGGGCGUG | |
| | GUGGGCGGAGUGGUAAGCGCCGUGAGCGGCGUGAGCAGCUUCAUGAGCAA | |
| | CCCCUUCGGUGCCCUGGCAGUGGGCCUGCUGGUGCUUGCUGGCCUGGUCG | |
| | CUGCCUUCUUCGCCUUCCGGUACGUGCUACAGCUGCAGCGGAAC | |
| 3′ UTR | UAAAGCUCCCCGGGGGCCUCGGUGGCCUAGCUUCUUGCCCCUUGGGCCUC | 88 |
| | CCCCCAGCCCCUCCUCCCCUUCCUGCAGGAGAUUGAGUGUAGUGACUAGU | |
| | GGUCUUUGAAUAAAGUCUGAGUGGGCGGC | |
| Corresponding amino acid | MRGGGLVCALVVGALVAAVASAAPAAPRASGGVAATVAANGGPASQPPPV | 54 |
| sequence | PSPATTKARKRKTKKPPKRPEATPPPDANATVAAGHATLRAHLREIKVEN | |
| | ADAQFYVCPPPTGATVVQFEQPRRCPTRPEGQNYTEGIAVVFKENIAPYK | |
| | FKATMYYKDVTVSQVWFGHRYSQFMGIFEDRAPVPFEEVIDKINAKGVCR | |
| | STAKYVRNNMETTAFHRDDHETDMELKPAKVATRTSRGWHTTDLKYNPSR | |
| | VEAFHRYGTTVNCIVEEVDARSVYPYDEFVLATGDFVYMSPFYGYREGSH | |
| | TEHTSYAADRFKQVDGFYARDLTTKARATSPTTRNLLTTPKFTVAWDWVP | |
| | KRPAVCTMTKWQEVDEMLRAEYGGSFRFSSDAISTTFTTNLTQYSLSRVD | |
| | LGDCIGRDAREAIDRMFARKYNATHIKVGQPQYYLATGGFLIAYQPLLSN | |
| | TLAELYVREYMREQDRKPRNATPAPLREAPSANASVERIKTTSSIEFARL | |
| | QFTYNHIQRHVNDMLGRIAVAWCELONHELTLWNEARKLNPNAIASATVG | |
| | RRVSARMLGDVMAVSTCVPVAPDNVIVONSMRVSSRPGTCYSRPLVSFRY | |
| | EDQGPLIEGOLGENNELRLTRDALEPCTVGHRRYFIFGGGYVYFEEYAYS | |
| | HQLSRADVTTVSTFIDLNITMLEDHEFVPLEVYTRHEIKDSGLLDYTEVQ | |
| | RRNQLHDLRFADIDTVIRADANAAMFAGLCAFFEGMGDLGRAVGKVVMGV | |
| | VGGVVSAVSGVSSFMSNPFGALAVGLLVLAGLVAAFFAFRYVLQLQRN | |
| PolyA tail | 100 nt | |
| HSV gC-F327AACT | | |
| SEQ ID NO: 105 consists of from 5′ end to 3′ end: 5′ UTR SEQ ID NO: 85, mRNA ORF | | |
| SEQ ID NO: 28, and 3′ UTR SEQ ID NO: 89. | | |
| Cap | C1 | |
| 5′ UTR | GAGGAAAUCGCAAAAUUUGCUCUUCGCGUUAGAUUUCUUUUAGUUUUCUC | 85 |
| | GCAACUAGCAAGCUUUUUGUUCUCGCC | |
| ORF of mRNA Construct | AUGGCACUUGGACGCGUCGGUCUGGCUGUCGGCCUGUGGGGCCUGCUGUG | 28 |
| (excluding the stop codon) | GGUGGGCGUGGUGGUGGUGCUGGCUAACGCCAGCCCCGGACGUACCAUCA | |
| | CCGUGGGGCCCAGAGGCAACGCCAGUAACGCCGCGCCAUCAGCCAGCCCG | |
| | AGAAACGCUAGUGCUCCCCGGACCACGCCAACUCCACCACAGCCCCGGAA | |
| | GGCCACCAAGAGCAAGGCCAGCACCGCCAAGCCCGCUCCACCUCCCAAGA | |
| | CCGGCCCACCCAAGACCAGCAGCGAGCCCGUGCGGUGCAACCGGCACGAU | |
| | CCUCUGGCCCGGUACGGCUCACGGGUGCAGAUCCGGUGCCGGUUCCCCAA | |
| | CAGCACCAGGACCGAGAGCCGGCUGCAGAUCUGGCGGUACGCCACCGCCA | |
| | CAGACGCCGAGAUCGGCACCGCCCCAAGCCUGGAGGAGGUGAUGGUGAAC | |
| | GUGUCUGCUCCACCUGGCGGCCAGCUGGUGUACGACAGCGCACCCAACCG | |
| | GACCGAUCCCCACGUGAUCUGGGCAGAAGGCGCUGGUCCUGGCGCUAGCC | |
| | CUCGACUGUACAGCGUGGUCGGCCCACUGGGCAGGCAGCGGCUGAUCAUC | |
| | GAGGAGCUGACCCUGGAGACCCAGGGCAUGUACUACUGGGUGUGGGGCCG | |
| | GACAGAUCGGCCUAGCGCCUACGGGACCUGGGUGCGGGUUCGCGUGUUCC | |
| | GGCCACCUAGCCUGACCAUCCAUCCCCACGCCGUGCUGGAGGGCCAGCCC | |
| | UUCAAAGCCACCUGUACCGCCGCCACCUACUACCCAGGCAACCGGGCCGA | |
| | GUUCGUGUGGUUCGAGGACGGACGGCGCGUGUUCGACCCCGCCCAGAUCC | |
| | ACACCCAGACCCAGGAGAACCCCGACGGCUUCAGCACCGUGAGCACGGUG | |
| | ACCAGCGCUGCCGUUGGAGGCCAAGGCCCUCCCAGAACCUUCACCUGCCA | |
| | GCUGACCUGGCACCGGGACAGCGUGAGCGCUUCUCGCCGGAACGCCAGCG | |
| | GAACUGCCAGCGUGCUGCCACGGCCCACCAUCACCAUGGAGUUCACCGGC | |
| | GACCACGCCGUGUGCACAGCCGGCUGCGUACCCGAGGGCGUGACCUUCGC | |
| | CUGGUUCCUGGGCGACGACAGCAGCCCCGCCGAGAAGGUGGCCGUGGCCA | |
| | GCCAGACCAGCUGUGGAAGACCCGGAACCGCCACCAUCCGGAGCACCCUG | |
| | CCCGUGAGCUACGAGCAGACCGAGUACAUCUGUCGGCUGGCCGGCUACCC | |
| | UGACGGCAUCCCCGUGUUGGAGCACCACGGCAGCCACCAGCCUCCUCCUC | |
| | GGGAUCCCACCGAGCGGCAGGUGAUCAGGGCCGUGGAGGGUGCAGGCAUC | |
| | GGCGUGGCCGUGCUGGUGGCCGUAGUGCUAGCCGGCACCGCCGUGGUAUA | |
| | CCUGACC | |
| 3′ UTR | UAAAGCUCCCCGGGGGCCUCGGUGGCCUAGCUUCUUGCCCCUUGGGCCUC | 89 |
| | CCCCCAGCCCCUCCUCCCCUUCCUGCAGGAUUGAGACUACGGGUGGUCUU | |
| | UGAAUAAAGUCUGAGUGGGCGGC | |
| Corresponding amino acid | MALGRVGLAVGLWGLLWVGVVVVLANASPGRTITVGPRGNASNAAPSASP | 63 |
| sequence | RNASAPRTTPTPPQPRKATKSKASTAKPAPPPKTGPPKTSSEPVRCNRHD | |
| | PLARYGSRVQIRCRFPNSTRTESRLQIWRYATATDAEIGTAPSLEEVMVN | |
| | VSAPPGGQLVYDSAPNRTDPHVIWAEGAGPGASPRLYSVVGPLGRORLII | |
| | EELTLETQGMYYWVWGRTDRPSAYGTWVRVRVFRPPSLTIHPHAVLEGQP | |
| | FKATCTAATYYPGNRAEFVWFEDGRRVFDPAQIHTQTQENPDGFSTVSTV | |
| | TSAAVGGQGPPRIFTCOLTWHRDSVSASRRNASGTASVLPRPTITMEFTG | |
| | DHAVCTAGCVPEGVTFAWFLGDDSSPAEKVAVASQTSCGRPGTATIRSTL | |
| | PVSYEQTEYICRLAGYPDGIPVLEHHGSHOPPPRDPTERQVIRAVEGAGI | |
| | GVAVLVAVVLAGTAVVYLT | |
| PolyA tail | 100 nt | |
| HSV gD | | |
| SEQ ID NO: 106 consists of from 5′ end to 3′ end: 5′ UTR SEQ ID NO: 85, mRNA ORF | | |
| SEQ ID NO: 4, and 3′ UTR SEQ ID NO: 90. | | |
| Cap | C1 | |
| 5′ UTR | GAGGAAAUCGCAAAAUUUGCUCUUCGCGUUAGAUUUCUUUUAGUUUUCUC | 85 |
| | GCAACUAGCAAGCUUUUUGUUCUCGCC | |
| ORF of mRNA Construct | AUGGGUCGGCUGACCAGCGGAGUUGGCACCGCCGCACUCCUGGUGGUGGC | 4 |
| (excluding the stop codon) | CGUAGGCCUGCGGGUGGUGUGCGCCAAGUACGCCCUGGCCGACCCAUCCC | |
| | UGAAGAUGGCCGACCCUAACCGGUUCCGGGGCAAGAAUCUGCCCGUGCUG | |
| | GAUCAGCUGACCGAUCCUCCUGGCGUGAAGCGGGUGUACCACAUCCAGCC | |
| | CAGCCUGGAGGAUCCCUUCCAGCCACCCUCCAUCCCCAUCACCGUUUACU | |
| | ACGCCGUGCUGGAGAGAGCCUGCCGCAGCGUGCUGCUGCACGCUCCAUCC | |
| | GAGGCGCCCCAGAUCGUGCGGGGCGCAAGCGACGAGGCCCGGAAGCACAC | |
| | CUACAACCUGACCAUCGCCUGGUACCGGAUGGGCGACAACUGCGCCAUCC | |
| | CUAUCACCGUGAUGGAGUACACCGAGUGCCCCUACAACAAGAGCCUGGGA | |
| | GUGUGCCCCAUCCGGACCCAGCCUCGGUGGUCCUACUACGACAGCUUCAG | |
| | CGCCGUGUCCGAGGACAACCUGGGCUUCCUGAUGCACGCCCCUGCCUUCG | |
| | AGACCGCCGGCACCUACCUGCGGCUGGUGAAGAUCAACGACUGGACCGAG | |
| | AUCACCCAGUUCAUCCUGGAGCACCGGGCAAGGGCCAGCUGCAAGUACGC | |
| | GCUGCCUCUGCGGAUCCCUCCCGCUGCUUGCCUGACCAGCAAGGCCUACC | |
| | AGCAGGGCGUGACCGUGGACAGCAUCGGCAUGCUGCCCAGGUUCAUCCCC | |
| | GAGAACCAGCGCACCGUGGCCCUGUACAGCCUGAAGAUCGCAGGCUGGCA | |
| | CGGACCCAAGCCUCCUUACACCUCCACCCUGCUGCCACCCGAGCUGAGCG | |
| | ACACCACCAACGCCACCCAGCCCGAGCUGGUGCCCGAGGACCCCGAGGAC | |
| | UCCGCCCUGCUGGAGGAUCCGGCCGGCACCGUAAGCUCCCAGAUCCCACC | |
| | CAACUGGCACAUCCCCAGCAUCCAGGACGUGGCCCCACAUCACGCGCCUG | |
| | CCGCUCCAAGCAACCCCGGCCUGAUCAUUGGAGCUCUGGCCGGGAGCACU | |
| | CUGGCGGUGCUGGUGAUCGGCGGCAUCGCCUUCUGGGUGCGUCGGAGAGC | |
| | CCAGAUGGCCCCUAAGCGGCUGCGGCUGCCACACAUACGGGACGACGACG | |
| | CGCCACCAUCCCACCAGCCCCUGUUCUAC | |
| 3′ UTR | UAAAGCUCCCCGGGGGCCUCGGUGGCCUAGCUUCUUGCCCCUUGGGCCUC | 90 |
| | CCCCCAGCCCCUCCUCCCCUUCCUGCAGCAUAGACACUACGUGGUCUUUG | |
| | AAUAAAGUCUGAGUGGGCGGC | |
| Corresponding amino acid | MGRLTSGVGTAALLVVAVGLRVVCAKYALADPSLKMADPNRFRGKNLPVL | 39 |
| sequence | DQLTDPPGVKRVYHIQPSLEDPFQPPSIPITVYYAVLERACRSVLLHAPS | |
| | EAPQIVRGASDEARKHTYNLTIAWYRMGDNCAIPITVMEYTECPYNKSLG | |
| | VCPIRTQPRWSYYDSFSAVSEDNLGFLMHAPAFETAGTYLRLVKINDWTE | |
| | ITQFILEHRARASCKYALPLRIPPAACLTSKAYQQGVTVDSIGMLPRFIP | |
| | ENQRTVALYSLKIAGWHGPKPPYTSTLLPPELSDTTNATQPELVPEDPED | |
| | SALLEDPAGTVSSQIPPNWHIPSIQDVAPHHAPAAPSNPGLIIGALAGST | |
| | LAVLVIGGIAFWVRRRAQMAPKRLRLPHIRDDDAPPSHOPLFY | |
| PolyA tail | 100 nt | |
| HSV ICPO Variant | | |
| SEQ ID NO: 107 consists of from 5′ end to 3′ end: 5′ UTR SEQ ID NO: 85, mRNA ORF | | |
| SEQ ID NO: 12, and 3′ UTR SEQ ID NO: 91. | | |
| Cap | C1 | |
| 5′ UTR | GAGGAAAUCGCAAAAUUUGCUCUUCGCGUUAGAUUUCUUUUAGUUUUCUC | 85 |
| | GCAACUAGCAAGCUUUUUGUUCUCGCC | |
| ORF of mRNA Construct | AUGAACACUCCCGUGGCCUACCUGAUCGUGGGUGUGACCGCCAGCGGCAG | 12 |
| (excluding the stop codon) | CUUCAGCACCAUCCCCAUCGUGAACGAUCCCCGGACGCGCGUCGAAGCAG | |
| | AAGCCGCCGUGAGAGCGGGUACCGCCGUGGACUUCAUCUGGACCGGCAAU | |
| | CCUCGGACCGCUCCUCGGAGCCUGAGCCUGGGCGGACACACCGUGAGGGC | |
| | CCUUAGCCCCACCCCUCCUUGGCCCGGAACCGACGACGAGGACGACGACC | |
| | UGGCCGACGUGGACUACGUGCCUCCGGCUCCUAGACGCGCUCCUAGAAGG | |
| | GGAGGUGGCGGUGCUGGAGCAACUAGGGGAACCAGCCAGCCUGCCGCUAC | |
| | AAGACCCGCACCUCCUGGUGCACCGCGGAGCAGCAGUAGCGGUGGCGCAC | |
| | CUCUGCGGGCAGGCGUUGGAUCAGGCUCCGGAGGCGGACCAGCUGUGGCC | |
| | GCCGUGGUUCCUCGGGUGGCCAGCCUACCACCUGCAGCCGGUGGAGGCAG | |
| | AGCGCAAGCUCGACGGGUGGGAGAGGACGCAGCUGCUGCCGAAGGCCGGA | |
| | CACCACCCGCUAGGCAACCACGGGCAGCCCAGGAGCCUCCCAUCGUGAUC | |
| | AGCGACAGCCCGCCACCAAGUCCCCGGAGACCUGCCGGACCUGGACCCCU | |
| | GAGCUUCGUGAGCAGCAGCAGCGCCCAGGUGUCAAGCGGUCCAGGAGGCG | |
| | GAGGCUUACCCCAGAGCAGCGGGAGGGCUGCAAGACCACGCGCUGCAGUG | |
| | GCUCCGCGGGUGAGAUCUCCGCCACGUGCCGCUGCAGCCCCUGUGGUGAG | |
| | CGCAUCCGCUGACGCUGCCGGUCCUGCACCUCCUGCCGUGCCCGUUGACG | |
| | CCCAUAGAGCUCCCCGGAGCCGGAUGACCCAGGCCCAGACCGACACCCAA | |
| | GCUCAAAGCCUGGGUCGUGCCGGUGCAACAGACGCCAGGGGCAGUGGUGG | |
| | ACCAGGUGCAGAGGGCGGGCCUGGAGUUCCACGGGGAACCAACACCCCUG | |
| | GUGCCGCUCCACACGCUGCCGAAGGGGCUGCUGCUGGCGGAGGCAGUGGA | |
| | GGUGGCUCUCCAGCCCCUGGACUGACCCGGUACCUGCCCAUCGCCGGCGU | |
| | GAGCAGCGUGGUGGCCCUGGCUCCCUACGUGAACAAGACCGUGACCGGCG | |
| | ACUGCUUACCCGUGCUGGACAUGGAGACCGGCCACAUCGGCGCCUACGUG | |
| | GUGCUGGUGGACCAGACCGGCAACGUGGCCGACCUCCUGAGAGCCGCCGC | |
| | ACCAGCUUGGAGCCGGAGAACCCUGCUGCCUGAGCACGCCCGGAACUGCG | |
| | UGCGGCCUCCCGACUACCCCACACCGCCUGCCAGCGAGUGGAACAGCCUG | |
| | UGGAUGACGCCCGUGGGCAACAUGCUGUUCGACCAGGGCACCCUGGUGGG | |
| | AGCCCUGGACUUCCACGGCCUGAGGAGCCGGCACCCUUGGAGCCGGGAGC | |
| | AAGGCGCACCC | |
| 3′ UTR | UAAAGCUCCCCGGGGGCCUCGGUGGCCUAGCUUCUUGCCCCUUGGGCCUC | 91 |
| | CCCCCAGCCCCUCCUCCCCUUCCUGCAGGAGAUUGAGUGUAGUGGUGGUC | |
| | UUUGAAUAAAGUCUGAGUGGGCGGC | |
| Corresponding amino acid | MNTPVAYLIVGVTASGSFSTIPIVNDPRTRVEAEAAVRAGTAVDFIWTGN | 47 |
| sequence | PRTAPRSLSLGGHTVRALSPTPPWPGTDDEDDDLADVDYVPPAPRRAPRR | |
| | GGGGAGATRGTSQPAATRPAPPGAPRSSSSGGAPLRAGVGSGSGGGPAVA | |
| | AVVPRVASLPPAAGGGRAQARRVGEDAAAAEGRTPPARQPRAAQEPPIVI | |
| | SDSPPPSPRRPAGPGPLSFVSSSSAQVSSGPGGGGLPQSSGRAARPRAAV | |
| | APRVRSPPRAAAAPVVSASADAAGPAPPAVPVDAHRAPRSRMTQAQTDTQ | |
| | AQSLGRAGATDARGSGGPGAEGGPGVPRGTNTPGAAPHAAEGAAAGGGSG | |
| | GGSPAPGLTRYLPIAGVSSVVALAPYVNKTVTGDCLPVLDMETGHIGAYV | |
| | VLVDQTGNVADLLRAAAPAWSRRTLLPEHARNCVRPPDYPTPPASEWNSL | |
| | WMTPVGNMLFDQGTLVGALDFHGLRSRHPWSREQGAP | |
| PolyA tail | 100 nt | |
| HSV ICP4 Variant | | |
| SEQ ID NO: 108 consists of from 5′ end to 3′ end: 5′ UTR SEQ ID NO: 85, mRNA ORF | | |
| SEQ ID NO: 14, and 3′ UTR SEQ ID NO: 92. | | |
| Cap | C1 | |
| 5′ UTR | GAGGAAAUCGCAAAAUUUGCUCUUCGCGUUAGAUUUCUUUUAGUUUUCUC | 85 |
| | GCAACUAGCAAGCUUUUUGUUCUCGCC | |
| ORF of mRNA Construct | AUGCGGGUGCUGUACGGUGGCCUGGGCGACUCAAGACCUGGCCUCUGGGG | 14 |
| (excluding the stop codon) | CGCACCUGAGGCAGAGGAAGCGCGGGCUCGUUUUGAGGCAUCUGGUGCCC | |
| | CUGCCCCUGUGUGGGCCCCAGAGCUGGGAGACGCCGCCCAGCAGUACGCC | |
| | CUGAUCACCCGGCUGCUGUACACACCCGACGCCGAGGCCAUGGGCUGGCU | |
| | GCAGAACCCUCGGGUGGCUCCAGGGGACGUGGCCCUGGACCAGGCCUGCU | |
| | UCAGAAUAAGCGGGGCCGCUCGGAACAGCAGCAGCUUCAUCAGCGGCAGC | |
| | GUUGCUCGAGCCGUGCCUCACCUGGGCUACGCAAUGGCCGCCGGCAGAUU | |
| | CGGCUGGGGCCUGGCUCACGUCGCUGCGGCAGUGGCUAUGAGCCGGCGGU | |
| | ACGACAGGGCCCAGAAGGGCUUCCUGUUGACAAGCCUGCGCCGCGCAUAC | |
| | GCUCCACUGUUAGCCCGCGAGAACGCCGCACUGACGGGAGCCCGGACUCC | |
| | UGACGACGGCGGAGACGCCAACAGACACGACGGGGACGACGCAAGAGGUA | |
| | AACCGCCCCUGCCCAGUGCUGCGGCCUCUCCCGCUGACGAGAGAGCCGUG | |
| | CCUGCAGGCUACGGCGCAGCUGGGGUGCUGGCAGCACUGGGCAGGCUGUC | |
| | AGCCGCCCCUGCUAGCGCUCCAGCUGGCGCUAGGGCUGAAGCAGGCAGGG | |
| | UGGCCGUGGAGUGCUUAGCCGCCUGUCGGGGCAUCCUGGAGGCCCUGGCC | |
| | GAAGGCUUUGACGGGGACCUGGCUGCGGUGCCUGGCUUGGCCGGUGCCAG | |
| | ACCUGCAGCACCUCCCCGACCUGGUCCAGCAGGCGCAGCAGCUCCUCCUC | |
| | ACGCUGACGCCCCACGCCUGAGAGCCUGGCUGCGGGAGCUGCGGUUCGUG | |
| | CGGGACGCCCUGGUGCUGAUGCGGCUGAGAGGCGACCUGCGUGUUGCUGG | |
| | CGGUUCCGAGGCAGCUGUGGCCGCAGUGAGAGCCGUUAGCCUGGUGGCCG | |
| | GCGCAUUAGGACCAGCCCUGCCACGUAGCCCGAGACUGCUGAGCAGCGAC | |
| | CUGCUGUUCCAGAAUCAGUCUUUGGGUGGAGGGUCCGGUGGAGGACCUCC | |
| | UCCAGGUGGAGCACCUGCCGCCUUCGGCCCUUUAAGGGCGUCUGGCCCUC | |
| | UGCGUAGAGCAGCCGCCUGGAUGCGGCAGGUGCCCGAUCCCGAGGACGUG | |
| | CGGGUGGUGAUCCUGUACAGUCCCCUGCCCGGCGAAGAUCUCGCAGCCGG | |
| | AAGAGCCGGAGGCGGACCACCACCUGAGUGGAGCGCCGAACGAGGUGGGC | |
| | UGAGCUGUCUUCUCGCCGCGCUGGGCAACAGACUCUGUGGACCCGCUACA | |
| | GCCGCCUGGGCUGGCAAUUGGACAGGUGCCCCGGACGUAUCCGCCCUGGG | |
| | CGCACAAGGCGUGCUGCUGCUGAGCACCCGGGAUCUGGCAUUCGCCGGCG | |
| | CCGUGGAGUUUCUGGGACUGCUGGCAGGUGCCUGUGACCGGCGGCUGAUC | |
| | GUGGUCAACGCAGUCAGAGCCGCUGACUGGCCUGCGGACGGACCUGUGGU | |
| | GAGCCGGCAGCACGCCUACCUGGCCUGCGAGGUGCUGCCAGCCGUGCAGU | |
| | GUGCAGUUCGGUGGCCUGCCGCCAGAGACCUGCGGCGGACCGUGUUAGCC | |
| | AGCGGCCGGGUUUUCGGUCCCGGAGUGUUCGCUCGGGUGGAAGCAGCCCA | |
| | CGCACGGCUGUACCCCGACGCCCCACCUCUGCGGCUGUGCAGAGGAGCAA | |
| | ACGUGCGGUACCGGGUGCGGACACGGUUCGGCCCAGACACCCUGGUGCCC | |
| | AUGUCACCCCGGGAGUACCGGAGAGCCGUGUUACCUGCCCUUGACGGAAG | |
| | GGCAGCCGCUUCUGGGGCCGGUGACGCUAUGGCUCCUGGCGCUCCCGACU | |
| | UCUGCGAGGACGAAGCCCACAGCCACCGAGCCUGCGCCAGGUGGGGUCUC | |
| | GGUGCCCCACUGAGACCCGUGUACGUGGCUCUGGGACGGGACGCUGUACG | |
| | GGGUGGGCCCGCUGAACUUAGAGGGCCACGGCGGGAGUUCUGUGCACGGG | |
| | CCCUGCUGGAG | |
| 3′ UTR | UAAAGCUCCCCGGGGGCCUCGGUGGCCUAGCUUCUUGCCCCUUGGGCCUC | 92 |
| | CCCCCAGCCCCUCCUCCCCUUCCUGCAGGAGAUUGAGUGUAGUGACGUGG | |
| | UCUUUGAAUAAAGUCUGAGUGGGCGGC | |
| Corresponding amino acid | MRVLYGGLGDSRPGLWGAPEAEEARARFEASGAPAPVWAPELGDAAQQYA | 49 |
| sequence | LITRLLYTPDAEAMGWLQNPRVAPGDVALDQACFRISGAARNSSSFISGS | |
| | VARAVPHLGYAMAAGRFGWGLAHVAAAVAMSRRYDRAQKGFLLTSLRRAY | |
| | APLLARENAALTGARTPDDGGDANRHDGDDARGKPPLPSAAASPADERAV | |
| | PAGYGAAGVLAALGRLSAAPASAPAGARAEAGRVAVECLAACRGILEALA | |
| | EGFDGDLAAVPGLAGARPAAPPRPGPAGAAAPPHADAPRLRAWLRELRFV | |
| | RDALVLMRLRGDLRVAGGSEAAVAAVRAVSLVAGALGPALPRSPRLLSSD | |
| | LLFONQSLGGGSGGGPPPGGAPAAFGPLRASGPLRRAAAWMRQVPDPEDV | |
| | RVVILYSPLPGEDLAAGRAGGGPPPEWSAERGGLSCLLAALGNRLCGPAT | |
| | AAWAGNWTGAPDVSALGAQGVLLLSTRDLAFAGAVEFLGLLAGACDRRLI | |
| | VVNAVRAADWPADGPVVSRQHAYLACEVLPAVQCAVRWPAARDLRRTVLA | |
| | SGRVFGPGVFARVEAAHARLYPDAPPLRLCRGANVRYRVRTRFGPDTLVP | |
| | MSPREYRRAVLPALDGRAAASGAGDAMAPGAPDFCEDEAHSHRACARWGL | |
| | GAPLRPVYVALGRDAVRGGPAELRGPRREFCARALLE | |
| PolyA tail | 100 nt | |

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A herpes simplex virus (HSV) messenger ribonucleic acid (mRNA) vaccine comprising
(a) an mRNA comprising an open reading frame encoding an HSV glycoprotein B (gB) that comprises a truncated C-terminus, relative to a wild-type HSV gB;
(b) an mRNA comprising an open reading frame encoding an HSV glycoprotein C (gC);
(c) an mRNA comprising an open reading frame encoding an HSV glycoprotein D (gD);
(d) an mRNA comprising an open reading frame encoding an HSV intracellular protein 0 (ICP0);
(e) an mRNA comprising an open reading frame encoding an HSV intracellular protein 4 (ICP4); and
(f) a lipid nanoparticle.

2. The HSV mRNA vaccine of claim 1, wherein the HSV gB comprises a truncated cytoplasmic tail.

3. The HSV mRNA vaccine of claim 1, wherein the HSV gB does not comprise a cytoplasmic tail.

4. The HSV mRNA vaccine of any one of claims 1-3, wherein the HSV gC comprises an F327A substitution and a truncated C-terminus, relative to a wild-type HSV gC.

5. The HSV mRNA vaccine of claim 4, wherein the HSV gC comprises a truncated cytoplasmic tail.

6. The HSV mRNA vaccine of claim 4, wherein the HSV gB and/or HSV gC does not comprise a cytoplasmic tail.

7. The HSV mRNA vaccine of any one of claims 1-6, wherein the HSV ICP0 lacks a nuclear localization signal and/or a RING finger domain.

8. The HSV mRNA vaccine of any one of claims 1-7, wherein the HSV ICP0 comprises a higher density of CD8+ T cell epitopes, relative to a wild-type HSV ICP0.

9. The HSV mRNA vaccine of any one of claims 1-8, wherein the HSV ICP4 lacks a nuclear localization signal and/or a RING finger domain.

10. The HSV mRNA vaccine of any one of claims 1-9, wherein the HSV ICP4 comprises a higher density of CD8+ T cell epitopes, relative to a wild-type HSV ICP4.

11. A herpes simplex virus (HSV) messenger ribonucleic acid (mRNA) vaccine comprising
(a) an mRNA comprising an open reading frame encoding an HSV glycoprotein B (gB), optionally wherein the gB comprises a truncated C-terminus, relative to a wild-type HSV gB;
(b) an mRNA comprising an open reading frame encoding a HSV glycoprotein C (gC) that comprises an F327A substitution, and a truncated C-terminus, relative to a wild-type HSV gC; and
(c) an mRNA comprising an open reading frame encoding a wild-type HSV glycoprotein D (gD);
(d) a lipid nanoparticle.

12. The HSV mRNA vaccine of claim 11 further comprising an mRNA comprising an open reading frame encoding a HSV intracellular protein 0 (ICP0) comprising a higher density of CD8+ T cell epitopes relative to a wild-type HSV ICP0.

13. The HSV mRNA vaccine of claim 11 or 12, further comprising an mRNA comprising an open reading frame encoding a HSV intracellular protein 4 (ICP4) comprising a higher density of CD8+ T cell epitopes relative to a wild-type HSV ICP4.

14. The HSV mRNA vaccine of any one of claims 11-13, wherein the HSV gB and/or HSV gC comprises a truncated cytoplasmic tail.

15. The HSV mRNA vaccine of any one of claims 11-13, wherein the HSV gB and/or HSV gC does not comprise a cytoplasmic tail.

16. A herpes simplex virus (HSV) messenger ribonucleic acid (mRNA) vaccine comprising
(a) an mRNA comprising an open reading frame encoding a HSV glycoprotein C (gC) that comprises an F327A substitution, and a truncated C-terminus, relative to a wild-type HSV gC;
(b) an mRNA comprising an open reading frame encoding a wild-type HSV glycoprotein D (gD);
(c) an mRNA comprising an open reading frame encoding a HSV intracellular protein 0 (ICP0) comprising a higher density of CD8+ T cell epitopes relative to a wild-type HSV ICP0;
(d) an mRNA comprising an open reading frame encoding a HSV intracellular protein 4 (ICP4) comprising a higher density of CD8+ T cell epitopes relative to a wild-type HSV ICP4; and
(e) a lipid nanoparticle.

17. The HSV mRNA vaccine of claim 16, wherein the HSV gC comprises a truncated cytoplasmic tail.

18. The HSV mRNA vaccine of claim 16 or 17, wherein the HSV gC does not comprise a cytoplasmic tail.

19. The HSV vaccine of any one of the preceding claims, wherein the vaccine induces a Th1-polarized CD4+ T cell-mediated immune response to the HSV gC, gB, and/or gD.

20. The HSV vaccine of any one of the preceding claims, wherein the vaccine elicits more Th1 cells that are specific to an antigen selected from HSV gB, gC, or gD, than Th2 cells specific to the antigen.

21. The HSV vaccine of any one of the preceding claims, wherein a population of CD4+ T cells specific to an antigen selected from HSV gB, gC, or gD, comprises more than 50% Th1 cells.

22. The HSV vaccine of any one of the preceding claims, wherein each of the HSV gB, gC, and gD comprises a transmembrane domain.

23. The HSV vaccine of any one of the preceding claims, wherein:
(a) the HSV gB has a length of about 798 amino acids;
(b) the HSV gC has a length of about 469 amino acids;
(c) the HSV ICP0 comprises a truncation in a nuclear localization signal, RING finger domain, and/or USP7-binding domain relative to a wild-type HSV ICP0; and/or
(d) the HSV ICP4 comprises a truncation in a nuclear localization signal and/or DNA-binding domain relative to a wild-type HSV ICP4.

24. The HSV vaccine of claim 23, wherein the HSV ICP0 does not comprise a nuclear localization signal, does not comprise a USP7-binding domain, and/or does not comprise a RING finger domain.

25. The HSV vaccine of claim 23 or 24, wherein the HSV ICP0 does not comprise a nuclear localization signal and/or comprises a truncated DNA-binding domain.

26. The HSV vaccine of any one of the preceding claims, wherein:
(a) the HSV gB comprises an amino acid sequence having at least 90%, at least 95%, or 100% identity to the amino acid sequence of SEQ ID NO: 54;
(b) the HSV gC comprises an amino acid sequence having at least 90%, at least 95%, or 100% identity to the amino acid sequence of SEQ ID NO: 63;
(c) the HSV gD comprises an amino acid sequence having at least 90%, at least 95%, or 100% identity to the amino acid sequence of SEQ ID NO: 39;
(d) the HSV ICP0 comprises an amino acid sequence having at least 90%, at least 95%, or 100% identity to the amino acid sequence of SEQ ID NO: 47; and/or
(e) the HSV ICP4 comprises an amino acid sequence having at least 90%, at least 95%, or 100% identity to the amino acid sequence of SEQ ID NO: 49.

27. The HSV vaccine of any one of the preceding claims, wherein the molar ratio of mRNA of (c) and (d) to the mRNA of (a), (b), and (c) is no more than 0.8:1.

28. The HSV vaccine of any one of the preceding claims, wherein the one or more mRNAs comprise a chemical modification.

29. The HSV vaccine of any one of the preceding claims, wherein 100% of the uracil nucleotides of the one or more mRNAs comprise a chemical modification.

30. The HSV vaccine of claim 28 or 29, wherein the chemical modification is 1-methylpseudouracil.

31. The HSV vaccine of any one of the preceding claims, wherein the lipid nanoparticle comprises an ionizable lipid, a neutral lipid, a sterol, and a PEG-modified lipid.

32. The HSV vaccine of claim 31, wherein the lipid nanoparticle comprises 40-50 mol % ionizable lipid, 5-15 mol % neutral lipid, 30-50 mol % sterol, and 0.5-3 mol % PEG-modified lipid.

33. The HSV vaccine of claim 31 or 32, wherein:
the ionizable lipid comprises a structure of Compound (I): 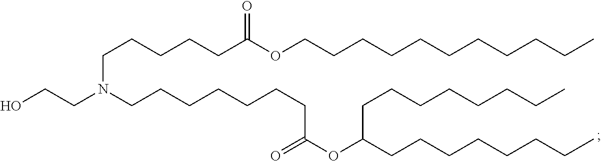 [SMILES: CCCCCCCCCCCOC(=O)CCCCCN(CCO)CCCCCCCC(=O)OC(CCCCCCCC)CCCCCCCC]
the neutral lipid is distearoylphosphatidylcholine (DSPC);
the sterol is cholesterol; and/or
the PEG-modified lipid is 1,2 dimyristoyl-sn-glycerol, methoxypolyethyleneglycol (PEG-DMG).

34. A method comprising administering to a subject the vaccine of any one of the preceding claims.

35. The method of claim 34, wherein the subject has an HSV infection or has been exposed to HSV.

36. The method of claim 34 or 35, wherein the vaccine is administered in an amount effective for preventing a latent or active HSV infection in the subject.

37. The method of any one of the preceding claims, wherein the vaccine is administered in an amount effective for preventing reactivation of a latent HSV infection in the subject, for preventing replication of HSV, reducing duration of an HSV infection in the subject, for reducing a number of replication-competent HSV particles in the subject, and/or for reducing a number of cells in the subject that comprise an HSV genome.

38. The method of any one of the preceding claims, wherein the vaccine induces a CD4+ T cell-mediated immune response to the HSV gB, gC, and/or gD, and the CD4+ T cells bind to one or more CD4+ T cell epitopes of the HSV gB, gC, or gD.

39. The method of claim 38, wherein at least 50% of the CD4+ T cells produce one or more cytokines selected from the group consisting of IFN-γ, IL-2, and TNF-α.

40. The method of claim 38 or 39, wherein fewer than 10% of the CD4+ T cells produce any one or more of IL-4, IL-5, IL-9, IL-10, or IL-13.

41. The method of any one of the preceding claims, wherein the vaccine induces a CD8+ T cell-mediated immune response to the HSV ICP0 and/or or ICP4, and the CD8+ T cells bind to one or more CD8+ T cell epitopes of the HSV ICP0 and or ICP4.

42. The method of claim 41, wherein the CD8+ T cells are cytotoxic.

43. A modified herpes simplex virus (HSV) intracellular protein 0 (ICP0) comprising fewer amino acids than a wild-type HSV ICP0.

44. The modified HSV ICP0 of claim 43, wherein the modified HSV ICP0 comprises a truncation in a nuclear localization signal relative to a wild-type HSV ICP0.

45. The modified HSV ICP0 of claim 43, wherein the modified HSV ICP0 does not comprise a nuclear localization signal.

46. The modified HSV ICP0 of any one of claims 43-45, wherein the modified HSV ICP0 comprises a truncation in a RING finger domain relative to a wild-type HSV ICP0.

47. The modified HSV ICP0 of any one of claims 43-45, wherein the modified HSV ICP0 does not comprise a RING finger domain.

48. The modified HSV ICP0 of any one of claims 43-47, wherein the modified HSV ICP0 comprises a truncation in a USP7-binding domain relative to the wild-type HSV ICP0.

49. The modified HSV ICP0 of any one of claims 43-47, wherein the modified HSV ICP0 does not comprise a USP7-binding domain.

50. The modified HSV ICP0 of any one of claims 43-49, wherein the modified HSV ICP0 comprises a linker between a first portion of the modified HSV ICP0 and a second portion of the modified HSV ICP0.

51. The modified HSV ICP0 of claim 50, wherein the linker comprises 2-10 glycine residues.

52. The modified HSV ICP0 of any one of claims 43-51, wherein the modified HSV ICP0 comprises an amino acid sequence having a length that is no more than 65% the length of the wild-type HSV IPC0.

53. The modified HSV ICP0 of any one of claims 43-52, wherein the modified HSV ICP0 amino acid sequence comprises at least 65% as many T cell epitopes as the wild-type HSV ICP0.

54. The modified HSV ICP0 of any one of claims 43-53, wherein the modified HSV ICP0 comprises about 487 amino acids.

55. A ribonucleic acid (RNA) comprising an open reading frame encoding the modified HSV ICP0 of any one of claims 43-54.

56. A modified herpes simplex virus (HSV) intracellular protein 4 (ICP4) comprising fewer amino acids than a wild-type HSV ICP4.

57. The modified HSV ICP4 of claim 56, wherein the modified HSV ICP4 comprises a truncation in a nuclear localization signal relative to the wild-type HSV ICP4.

58. The modified HSV ICP4 of claim 56, wherein the modified HSV ICP4 does not comprise a nuclear localization signal.

59. The modified HSV ICP4 of any one of claims 56-58, wherein the modified HSV ICP4 comprises a truncation in a DNA-binding domain relative to the wild-type HSV ICP4.

60. The modified HSV ICP4 of claim 59, wherein the modified HSV ICP4 does not comprise a DNA-binding domain.

61. The modified HSV ICP4 of any one of claims 56-60, wherein the modified HSV ICP4 comprises a linker between a first portion of the modified HSV ICP4 and a second portion of the modified HSV ICP4.

62. The modified HSV ICP4 of claim 61, wherein the linker comprises 2-10 glycine residues.

63. The modified HSV ICP4 of any one of claims 56-62, wherein the modified HSV ICP4 comprises an amino acid sequence having a length that is no more than 65% the length of the wild-type HSV IPC4.

64. The modified HSV ICP4 of any one of claims 56-63, wherein the modified HSV ICP4 amino acid sequence comprises at least 65% as many T cell epitopes as the wild-type HSV ICP4.

65. The modified HSV ICP4 of any one of claims 56-64, wherein the modified HSV ICP4 comprises about 687 amino acids.

66. A ribonucleic acid (RNA) comprising an open reading frame encoding the modified HSV ICP4 of any one of claims 56-65.

67. A herpes simplex virus (HSV) protein comprising an amino acid sequence having at least 90%, at least 95%, at least 96%, at least 97%, at least 98%, at least 99%, or 100% identity to the amino acid sequence of any one of SEQ ID NOs: 54, 63, 47, and 49.

68. A messenger ribonucleic acid (mRNA) comprising an open reading frame encoding the HSV protein of claim 67.

69. A messenger ribonucleic acid (mRNA) comprising an open reading frame comprising a nucleic acid sequence having at least 90%, at least 95%, at least 96%, at least 97%, at least 98%, at least 99%, or 100% identity to the nucleic acid sequence of any one of SEQ ID NOs: 19, 28, 4, 12, and 14.

70. The mRNA of claim 68 or 69, wherein the mRNA comprises a chemical modification.

71. The mRNA of any one of claims 68-70, wherein 100% of the uracil nucleotides of the mRNA comprises a chemical modification.

72. The mRNA of claim 71, wherein the chemical modification is 1-methylpseudouracil.


SEQUENCE LISTING (121 sequences)

SEQ ID NO: 1 - RNA, 2703 nt, unidentified
  source 1..2703: mol_type = genomic RNA; note = Herpes simplex virus
atgcggggtggcggactggtgtgcgccctggtggtcggtgcacttgtggccgctgtggctagcgctgcaccagccgcacccagagccagcggaggtgtggccgcgaccgttgcagccaacggaggcccggcttctcagcctcctcctgtgcccagccccgctaccaccaaggcccgtaagcggaagaccaagaagcctcccaagcggcccgaggctacaccaccacccgacgccaacgcaactgttgctgctggccacgccaccctgagggcccacctgcgggagatcaaggtggagaacgccgacgcccagttctacgtgtgtccacctccaaccggcgccaccgtggtacagttcgagcaacctcgccggtgccccactcgacccgagggccagaactacaccgagggcatcgccgtggtgttcaaggagaacatcgccccatacaagttcaaggccaccatgtactacaaggacgtgaccgtgagccaggtgtggttcggccaccggtacagccagttcatgggcatcttcgaggatcgggccccagtgcccttcgaggaggtgatcgacaagatcaacgccaagggcgtgtgccggagcaccgccaagtacgtgcggaacaacatggagaccaccgccttccaccgggacgaccacgagaccgacatggagctgaagcccgccaaggtggccacacggacaagccggggctggcacaccaccgacctgaagtacaacccaagccgcgtggaggcgttccaccggtacggcaccaccgtgaactgcatcgtggaggaggtggacgcccggagcgtgtacccctacgacgaattcgtgctggccaccggcgacttcgtgtacatgagccccttctacggctaccgggagggcagccacaccgagcacaccagctacgccgccgaccggttcaagcaggtggacggcttctacgcccgggacctgaccactaaagcccgggccacctcaccaaccacccggaacctgctgaccaccccaaagttcaccgtggcctgggactgggtgcccaagcgacccgccgtgtgcaccatgaccaagtggcaggaagtggacgagatgctgcgggccgaatacggcggcagcttccggttcagcagcgacgccatcagcaccaccttcaccaccaacctgacccagtacagcctgagccgggtggatctgggcgactgcatcggcagagacgctcgcgaggccatcgaccggatgttcgcccggaagtacaacgccacccacatcaaggtgggccagccccagtactacctcgccaccggcggcttcctgatcgcctaccagcccttgctgagcaacaccctggccgagctgtacgtgcgggagtacatgcgggagcaggaccggaagccccggaacgccacacccgcccctctgagagaagccccttccgccaacgccagcgtggagcggatcaagaccaccagcagcatcgagttcgcccggctgcagttcacctacaaccacatccagcggcacgtgaacgacatgctgggtcggatcgccgtggcttggtgcgagctgcagaaccacgagctgaccctgtggaacgaggcccggaagctgaaccccaacgccatcgccagcgctactgtgggccggagggtaagcgctcggatgctgggcgacgtgatggccgtgagcacgtgtgtgcccgtggccccagacaacgtgatcgtgcagaacagcatgcgggtgagtagccggcctggaacctgctacagccggcccctggtgtctttccggtacgaggaccagggtcccctgatcgagggccagctgggcgagaacaacgagctgcggctgactcgcgacgctctggagccctgcaccgtgggccacagacggtacttcatcttcggcggcggctacgtgtacttcgaggagtacgcctacagccaccagctgagccgggccgacgtgaccaccgtgagcaccttcatcgacctgaacatcaccatgctggaggaccacgagttcgtgcccctggaggtgtacacccggcacgagatcaaggacagcggcctgctggattacaccgaggtgcagcggcggaaccagctgcacgacctgcggttcgccgacatcgacaccgtgatacgggccgacgcaaacgccgccatgttcgccggcctgtgcgccttcttcgagggcatgggcgacctgggcagagccgtgggcaaggtggtgatgggcgtggtgggcggagtggtaagcgccgtgagcggcgtgagcagcttcatgagcaaccccttcggtgccctggcagtgggcctgctggtgcttgctggcctggtcgctgccttcttcgccttccggtacgtgctacagctgcagcggaaccccatgaaggccctgtaccctctgaccaccaaggagctgaagacctcagaccctggcggcgttggaggagagggtgaggagggagcagagggcggaggcttcgacgaggccaagctggctgaggcccgggagatgatccggtacatggccctggtgagcgccatggagcggaccgagcacaaggccagaaagaagggcaccagcgccctgctgagcagcaaggtgaccaacatggtgctgcggaagcggaacaaggcccggtactcacccctgcacaacgaggacgaggccggcgacgaagacgagctg

SEQ ID NO: 2 - RNA, 2703 nt, synthetic construct
  source 1..2703: mol_type = other RNA
atgcggggtggcggactggtgtgcgccctggtggtcggtgcacttgtggccgctgtggctagcgctgcaccagccgcacccagagccagcggaggtgtggccgcgaccgttgcagccaacggaggcccggcttctcagcctcctcctgtgcccagccccgctaccaccaaggcccgtaagcggaagaccaagaagcctcccaagcggcccgaggctacaccaccacccgacgccaacgcaactgttgctgctggccacgccaccctgagggcccacctgcgggagatcaaggtggagaacgccgacgcccagttctacgtgtgtccacctccaaccggcgccaccgtggtacagttcgagcaacctcgccggtgccccactcgacccgagggccagaactacaccgagggcatcgccgtggtgttcaaggagaacatcgccccatacaagttcaaggccaccatgtactacaaggacgtgaccgtgagccaggtgtggttcggccaccggtacagccagttcatgggcatcttcgaggatcgggccccagtgcccttcgaggaggtgatcgacaagatcaacgccaagggcgtgtgccggagcaccgccaagtacgtgcggaacaacatggagaccaccgccttccaccgggacgaccacgagaccgacatggagctgaagcccgccaaggtggccacacggacaagccggggctggcacaccaccgacctgaagtacaacccaagccgcgtggaggcgttccaccggtacggcaccaccgtgaactgcatcgtggaggaggtggacgcccggagcgtgtacccctacgacgaattcgtgctggccaccggcgacttcgtgtacatgagccccttctacggctaccgggagggcagccacaccgagcacaccagctacgccgccgaccggttcaagcaggtggacggcttctacgcccgggacctgaccactaaagcccgggccacctcaccaaccacccggaacctgctgaccaccccaaagttcaccgtggcctgggactgggtgcccaagcgacccgccgtgtgcaccatgaccaagtggcaggaagtggacgagatgctgcgggccgaatacggcggcagcttccggttcagcagcgacgccatcagcaccaccttcaccaccaacctgacccagtacagcctgagccgggtggatctgggcgactgcatcggcagagacgctcgcgaggccatcgaccggatgttcgcccggaagtacaacgccacccacatcaaggtgggccagccccagtactacctcgccaccggcggcttcctgatcgcctaccagcccttgctgagcaacaccctggccgagctgtacgtgcgggagtacatgcgggagcaggaccggaagccccggaacgccacacccgcccctctgagagaagccccttccgccaacgccagcgtggagcggatcaagaccaccagcagcatcgagttcgcccggctgcagttcacctacaaccacatccagcggcctgtgaacgacatgctgggtcggatcgccgtggcttggtgcgagctgcagaaccacgagctgaccctgtggaacgaggcccggaagctgaaccccaacgccatcgccagcgctactgtgggccggagggtaagcgctcggatgctgggcgacgtgatggccgtgagcacgtgtgtgcccgtggccccagacaacgtgatcgtgcagaacagcatgcgggtgagtagccggcctggaacctgctacagccggcccctggtgtctttccggtacgaggaccagggtcccctgatcgagggccagctgggcgagaacaacgagctgcggctgactcgcgacgctctggagccctgcaccgtgggccacagacggtacttcatcttcggcggcggctacgtgtacttcgaggagtacgcctacagccaccagctgagccgggccgacgtgaccaccgtgagcaccttcatcgacctgaacatcaccatgctggaggaccacgagttcgtgcccctggaggtgtacacccggcacgagatcaaggacagcggcctgctggattacaccgaggtgcagcggcggaaccagctgcacgacctgcggttcgccgacatcgacaccgtgatacgggccgacgcaaacgccgccatgttcgccggcctgtgcgccttcttcgagggcatgggcgacctgggcagagccgtgggcaaggtggtgatgggcgtggtgggcggagtggtaagcgccgtgagcggcgtgagcagcttcatgagcaaccccttcggtgccctggcagtgggcctgctggtgcttgctggcctggtcgctgccttcttcgccttccggtacgtgctacagctgcagcggaaccccatgaaggccctgtaccctctgaccaccaaggagctgaagacctcagaccctggcggcgttggaggagagggtgaggagggagcagagggcggaggcttcgacgaggccaagctggctgaggcccgggagatgatccggtacatggccctggtgagcgccatggagcggaccgagcacaaggccagaaagaagggcaccagcgccctgctgagcagcaaggtgaccaacatggtgctgcggaagcggaacaaggcccggtactcacccctgcacaacgaggacgaggccggcgacgaagacgagctg

SEQ ID NO: 3 - RNA, 1440 nt, synthetic construct
  source 1..1440: mol_type = other RNA
atggcacttggacgcgtcggtctggctgtcggcctgtggggcctgctgtgggtgggcgtggtggtggtgctggctaacgccagccccggacgtaccatcaccgtggggcccagaggcaacgccagtaacgccgcgccatcagccagcccgagaaacgctagtgctccccggaccacgccaactccaccacagccccggaaggccaccaagagcaaggccagcaccgccaagcccgctccacctcccaagaccggcccacccaagaccagcagcgagcccgtgcggtgcaaccggcacgatcctctggcccggtacggctcacgggtgcagatccggtgccggttccccaacagcaccaggaccgagagccggctgcagatctggcggtacgccaccgccacagacgccgagatcggcaccgccccaagcctggaggaggtgatggtgaacgtgtctgctccacctggcggccagctggtgtacgacagcgcacccaaccggaccgatccccacgtgatctgggcagaaggcgctggtcctggcgctagccctcgactgtacagcgtggtcggcccactgggcaggcagcggctgatcatcgaggagctgaccctggagacccagggcatgtactactgggtgtggggccggacagatcggcctagcgcctacgggacctgggtgcgggttcgcgtgttccggccacctagcctgaccatccatccccacgccgtgctggagggccagcccttcaaagccacctgtaccgccgccacctactacccaggcaaccgggccgagttcgtgtggttcgaggacggacggcgcgtgttcgaccccgcccagatccacacccagacccaggagaaccccgacggcttcagcaccgtgagcacggtgaccagcgctgccgttggaggccaaggccctcccagaaccttcacctgccagctgacctggcaccgggacagcgtgagcgcttctcgccggaacgccagcggaactgccagcgtgctgccacggcccaccatcaccatggagttcaccggcgaccacgccgtgtgcacagccggctgcgtacccgagggcgtgaccttcgcctggttcctgggcgacgacagcagccccgccgagaaggtggccgtggccagccagaccagctgtggaagacccggaaccgccaccatccggagcaccctgcccgtgagctacgagcagaccgagtacatctgtcggctggccggctaccctgacggcatccccgtgttggagcaccacggcagccaccagcctcctcctcgggatcccaccgagcggcaggtgatcagggccgtggagggtgcaggcatcggcgtggccgtgctggtggccgtagtgctagccggcaccgccgtggtatacctgacccacgccagcagcgtgcgctaccggagactgcgg

SEQ ID NO: 4 - RNA, 1179 nt, unidentified
  source 1..1179: mol_type = genomic RNA; note = Herpes simplex virus
atgggtcggctgaccagcggagttggcaccgccgcactcctggtggtggccgtaggcctgcgggtggtgtgcgccaagtacgccctggccgacccatccctgaagatggccgaccctaaccggttccggggcaagaatctgcccgtgctggatcagctgaccgatcctcctggcgtgaagcgggtgtaccacatccagcccagcctggaggatcccttccagccaccctccatccccatcaccgtttactacgccgtgctggagagagcctgccgcagcgtgctgctgcacgctccatccgaggcgccccagatcgtgcggggcgcaagcgacgaggcccggaagcacacctacaacctgaccatcgcctggtaccggatgggcgacaactgcgccatccctatcaccgtgatggagtacaccgagtgcccctacaacaagagcctgggagtgtgccccatccggacccagcctcggtggtcctactacgacagcttcagcgccgtgtccgaggacaacctgggcttcctgatgcacgcccctgccttcgagaccgccggcacctacctgcggctggtgaagatcaacgactggaccgagatcacccagttcatcctggagcaccgggcaagggccagctgcaagtacgcgctgcctctgcggatccctcccgctgcttgcctgaccagcaaggcctaccagcagggcgtgaccgtggacagcatcggcatgctgcccaggttcatccccgagaaccagcgcaccgtggccctgtacagcctgaagatcgcaggctggcacggacccaagcctccttacacctccaccctgctgccacccgagctgagcgacaccaccaacgccacccagcccgagctggtgcccgaggaccccgaggactccgccctgctggaggatccggccggcaccgtaagctcccagatcccacccaactggcacatccccagcatccaggacgtggccccacatcacgcgcctgccgctccaagcaaccccggcctgatcattggagctctggccgggagcactctggcggtgctggtgatcggcggcatcgccttctgggtgcgtcggagagcccagatggcccctaagcggctgcggctgccacacatacgggacgacgacgcgccaccatcccaccagcccctgttctac

SEQ ID NO: 5 - RNA, 1635 nt, synthetic construct
  source 1..1635: mol_type = other RNA
atggccagaggagccggcctggtgttcttcgtgggcgtgtgggtggtgtcctgcctggccgcagctcccagaaccagctggaagcgggtgaccagcggcgaggacgtggtgttactgccagctccagccgagcggactcgggcccacaagctgctttgggcagccgagccactggacgcctgcgggccattacggcctagctgggtggctctgtggccacctagacgggtgctggagaccgtggtggacgcagcctgcatgcgggcaccagagcccctggccatcgcctactctccacccttcccagccggcgacgaaggcctgtacagcgagctggcttggagagaccgggtggccgtggtgaacgagagcctggtgatctacggcgccctggagaccgacagcggcctgtacaccctgagcgtggtgggcctgagcgacgaggcccggcaagtggccagcgtggtgctggtggttgagcccgcccctgtgccaacgcccactcccgacgactacgacgaggaggacgacgcaggcgtgagcgagcggaccccagtgagcgtgcctccacccactcctcctcggagacctcccgtggctccacctacccatccccgggtgatccccgaggtgagccacgtgcggggcgtgaccgtgcacatggagacacccgaggccatcctgttcgcccctggcgagaccttcggcaccaacgtgagcatccacgccatagcccacgacgacggcccttacgccatggacgtcgtgtggatgcggttcgacgtgcccagcagctgcgccgagatgcggatctacgaggcctgcctgtaccatccccagctgcccgagtgcctgagccccgctgacgcaccctgcgccgtgagcagctgggcctaccggctggctgtgcggagctacgccgggtgtagccggacaacgcctccgccacggtgcttcgccgaggcccggatggaacctgttcccggcctggcctggcttgctagcaccgtgaacctggagttccagcacgccagcccacaacacgccggcctgtacctgtgcgtggtgtacgtggacgaccacatccacgcctggggccacatgaccatcagcaccgccgcccagtaccggaacgccgtggtggagcagcatctgccccagaggcagcccgagccagtggagcccacgagaccacacgtgcgggcacctcaccctgctcccagcgcacgtgggccactgcggctgggagctgtgctgggcgccgctctgctgctggcagccctgggactgagcgcctgggcctgcatgacctgttggaggaggcggtcttggcgcgcagtgaagagccgggccagtgccacaggcccaacctacatccgggtggccgacagcgagctgtacgccgactggagcagcgacagcgagggcgaacgggacggcagcctgtggcaggatccaccagagcggcctgacagccccagcaccaacggcagcggcttcgagatcctgagcccaaccgcccctagcgtgtaccctcacagcgagggacggaagagcagacggcccctgaccaccttcgggtctggcagcccaggccggagacacagccaggccagctaccccagcgtgctgtgg

SEQ ID NO: 6 - RNA, 1251 nt, synthetic construct
  source 1..1251: mol_type = other RNA
atggccagaggagccggcctggtgttcttcgtgggcgtgtgggtggtgtcctgcctggccgcagctcccagaaccagctggaagcgggtgaccagcggcgaggacgtggtgttactgccagctccagctggccctgaggagcggactcgggcccacaagctgctttgggcagccgagccactggacgcctgcgggccattacggcctagctgggtggctctgtggccacctagacgggtgctggagaccgtggtggacgcagcctgcatgcgggcaccagagcccctggccatcgcctactctccacccttcccagccggcgacgaaggcctgtacagcgagctggcttggagagaccgggtggccgtggtgaacgagagcctggtgatctacggcgccctggagaccgacagcggcctgtacaccctgagcgtggtgggcctgagcgacgaggcccggcaagtggccagcgtggtgctggtggttgagcccgcccctgtgccaacgcccactcccgacgactacgacgaggaggacgacgcaggcgtgagcgagcggaccccagtgagcgtgcctccacccactcctcctcggagacctcccgtggctccacctacccatccccgggtgatccccgaggtgagccacgtgcggggcgtgaccgtgcacatggagacacccgaggccatcctgttcgcccctggcgagaccttcggcaccaacgtgagcatccacgccatagcccacgacgacggcccttacgccatggacgtcgtgtggatgcggttcgacgtgcccagcagctgcgccgagatgcggatctacgaggcctgcctgtaccatccccagctgcccgagtgcctgagccccgctgacgcaccctgcgccgtgagcagctgggcctaccggctggctgtgcggagctacgccgggtgtagccggacaacgcctccgccacggtgcttcgccgaggcccggatggaacctgttcccggcctggcctggcttgctagcaccgtgaacctggagttccagcacgccagcccacaacacgccggcctgtacctgtgcgtggtgtacgtggacgaccacatccacgcctggggccacatgaccatcagcaccgccgcccagtaccggaacgccgtggtggagcagcatctgccccagaggcagcccgagccagtggagcccacgagaccacacgtgcgggcacctccacctgctcccagcgcacgtgggccactgcgg

SEQ ID NO: 7 - RNA, 2514 nt, synthetic construct
  source 1..2514: mol_type = other RNA
atgggccccggtctgtgggtggtgatgggcgtgctggttggcgtggctggcggacacgacacctactggaccgagcagatcgacccctggttcctgcacggcctgggcttggctcggacctactggcgggacaccaacaccggacggctgtggctgcccaacaccccagacgccagcgatccccaaagaggccggctggctccacctggcgagctgaacctgaccaccgccagcgtgcccatgctgcggtggtacgccgagcggttctgcttcgtgctggtgaccacggccgagtttccacgggacccaggccagctgctgtacatccccaagacctacctgctgggtagaccccggaacgccagccttcccgagctgcctgaggctggccccacctcacgaccacccgccgaggtgacccagctgaagggcctgagccacaatcccggagccagcgctctgctgcggagccgggcttgggtgaccttcgccgcagctcccgatagggagggcctgacattcccgagaggcgacgacggggcaaccgaaagacacccagacggccgcagaaacgctccacctcccggaccaccagcaggtacgccccggcatcccaccaccaacctgagcatcgcccacctgcacaacgccagcgtgacttggctggcggccagaggtctactgcggacgcccggcagatacgtgtacctgagcccctctgccagcacctggccagtgggcgtgtggaccaccggtggcctggccttcggttgcgacgccgctctcgtgcgagcccggtacggcaagggcttcatgggcctggttatcagcatgcgggacagtcctcccgccgagatcatcgtggtgcccgccgacaagaccctggcccgggttggcaaccccaccgacgagaacgcacccgcagtgctgccagggcctccagctggccctcggtaccgggtgttcgtgctgggagcgccaaccccagccgacaacggcagcgcgttggacgccttacgacgggtggccggctatcccgaggagagcaccaactacgcccagtacatgagccgggcctacgccgagtttctgggcgaggacccagggagcggcaccgacgcaaggcccagcctgttttggcgactggcaggcctcctggccagcagcggcttcgccttcgtgaacgccgcacacgcgcacgacgccatccggctgagcgacctgctgggcttcctggcccactctcgggtgctcgcaggccttgctgctcggggagcagccggttgtgccgccgacagcgtgttcctgaacgtgagcgtgcttgaccctgcggccaggctgagactggaggcccggctgggacacctggtggccgccatcctggagcgggagcagagcctggcagcccacgctctgggctaccagctggcctttgttctggacagcccagccgcctacggagccgttgccccttccgcagccagactgatcgacgccctgtacgccgaattcttaggtggacgggccctgaccgcaccaatggtgcggcgggccctgttctacgccaccgctgtcctgcgggcaccattccttgccggagccccatctgccgagcagcgggagcgtgccagaaggggtctgctgatcaccaccgccctgtgcaccagcgacgttgctgccgccacccacgctgatttacgagctgccctcgcacggaccgaccaccagaagaacctgttctggctgcccgaccacttctctccttgcgccgcgagcctacggttcgacctggccgaaggcggcttcatcctggacgccctcgccatggccacccggagcgacatccccgccgacgtgatggcccaacagacccggggagtggccagtgcactgacccggtgggcccactacaacgccctgatccgggccttcgtgcccgaggccacccaccagtgcagcggccccagccacaacgccgagccccggatcctggtgcccatcacccacaacgctagctacgtggtgacccacactcccttgccccggggaatcggctacaagctgaccggcgtggacgttcggcggcccctgttcatcacctacctgaccgccacctgtgaggggcacgcccgagagatcgagcccaagcggctggtgcggaccgagaatcggcgagacctgggcctggtgggtgccgtgttcctgcggtacacacccgccggcgaggtgatgagcgtgctgctggtggacaccgacgccacccagcagcaattggcccaaggccccgttgccggcacacccaacgtgttcagcagcgacgtgcccagcgtggccctgctgctgttccccaacggcaccgtgatccacctgctggccttcgacaccctgcccatcgccaccatcgccccagggttcctggctgcgagcgctctgggagtggtgatgatcaccgccgccttagccggcatcctgcgggttgtgcggacctgcgtgcccttcctgtggcggcgggag

SEQ ID NO: 8 - RNA, 672 nt, synthetic construct
  source 1..672: mol_type = other RNA
atgggcttcgtgtgcctgttcggcctggtggtgatgggcgcttggggtgcctggggcggtagccaggccaccgagtacgtgctgcggagcgtgatcgccaaggaggtgggcgacatcctgcgggtgccctgcatgcggacacccgccgacgacgtgagctggcggtacgaggcccctagcgtgatcgactacgcccggatcgacggcatcttcctgcggtaccactgccccggcctggacaccttcctgtgggaccggcacgcccagagagcctacctggtgaaccccttcctgttcgccgccggcttcctggaggacctgagccacagcgtgttccccgccgacacccaggagaccacaacccggcgggctctgtacaaggagatccgggacgccttaggcagccggaagcaggccgtgagccacgcaccagtgcgggctggctgcgtgaacttcgactacagccggaccaggcggtgtgtgggcagacgggaccttcggccagccaacaccaccagcacctgggagcctcctgtgagcagcgacgacgaggccagcagccagagcaagcccttagccacccagccacctgtgctggccctgtcaaacgctccacccaggcgggtgagccctactcgcggacggcgaaggcatacccggctgaggcggaac

SEQ ID NO: 9 - RNA, 627 nt, synthetic construct
  source 1..627: mol_type = other RNA
atggcctggggcggtagccaggccaccgagtacgtgctgcggagcgtgatcgccaaggaggtgggcgacatcctgcgggtgccctgcatgcggacacccgccgacgacgtgagctggcggtacgaggcccctagcgtgatcgactacgcccggatcgacggcatcttcctgcggtaccactgccccggcctggacaccttcctgtgggaccggcacgcccagagagcctacctggtgaaccccttcctgttcgccgccggcttcctggaggacctgagccacagcgtgttccccgccgacacccaggagaccacaacccggcgggctctgtacaaggagatccgggacgccttaggcagccggaagcaggccgtgagccacgcaccagtgcgggctggctgcgtgaacttcgactacagccggaccaggcggtgtgtgggcagacgggaccttcggccagccaacaccaccagcacctgggagcctcctgtgagcagcgacgacgaggccagcagccagagcaagcccttagccacccagccacctgtgctggccctgtcaaacgctccacccaggcgggtgagccctactcgcggacggcgaaggcatacccggctgaggcggaac

SEQ ID NO: 10 - RNA, 3228 nt, synthetic construct
  source 1..3228: mol_type = other RNA
atgggcttcgtgtgcctgttcggcctggtggtgatgggcgcttggggtgcctggggcggtagccaggccaccgagtacgtgctgcggagcgtgatcgccaaggaggtgggcgacatcctgcgggtgccctgcatgcggacacccgccgacgacgtgagctggcggtacgaggcccctagcgtgatcgactacgcccggatcgacggcatcttcctgcggtaccactgccccggcctggacaccttcctgtgggaccggcacgcccagagagcctacctggtgaaccccttcctgttcgccgccggcttcctggaggacctgagccacagcgtgttccccgccgacacccaggagaccacaacccggcgggctctgtacaaggagatccgggacgccttaggcagccggaagcaggccgtgagccacgcaccagtgcgggctggctgcgtgaacttcgactacagccggaccaggcggtgtgtgggcagacgggaccttcggccagccaacaccaccagcacctgggagcctcctgtgagcagcgacgacgaggccagcagccagagcaagcccttagccacccagccacctgtgctggccctgtcaaacgctccacccaggcgggtgagccctactcgcggacggcgaaggcatacccggagaagacggagaggcagcggcggatctggcagcggcggaagcagcggtggaggaagcggcagcggaggctcaggcggctcagggtctggcggcagaaggaggcggcggcacgacacctactggaccgagcagatcgacccctggttcctgcacggcctgggcttggctcggacctactggcgggacaccaacaccggacggctgtggctgcccaacaccccagacgccagcgatccccaaagaggccggctggctccacctggcgagctgaacctgaccaccgccagcgtgcccatgctgcggtggtacgccgagcggttctgcttcgtgctggtgaccacggccgagtttccacgggacccaggccagctgctgtacatccccaagacctacctgctgggtagaccccggaacgccagccttcccgagctgcctgaggctggccccacctcacgaccacccgccgaggtgacccagctgaagggcctgagccacaatcccggagccagcgctctgctgcggagccgggcttgggtgaccttcgccgcagctcccgatagggagggcctgacattcccgagaggcgacgacggggcaaccgaaagacacccagacggccgcagaaacgctccacctcccggaccaccagcaggtacgccccggcatcccaccaccaacctgagcatcgcccacctgcacaacgccagcgtgacttggctggcggccagaggtctactgcggacgcccggcagatacgtgtacctgagcccctctgccagcacctggccagtgggcgtgtggaccaccggtggcctggccttcggttgcgacgccgctctcgtgcgagcccggtacggcaagggcttcatgggcctggttatcagcatgcgggacagtcctcccgccgagatcatcgtggtgcccgccgacaagaccctggcccgggttggcaaccccaccgacgagaacgcacccgcagtgctgccagggcctccagctggccctcggtaccgggtgttcgtgctgggagcgccaaccccagccgacaacggcagcgcgttggacgccttacgacgggtggccggctatcccgaggagagcaccaactacgcccagtacatgagccgggcctacgccgagtttctgggcgaggacccagggagcggcaccgacgcaaggcccagcctgttttggcgactggcaggcctcctggccagcagcggcttcgccttcgtgaacgccgcacacgcgcacgacgccatccggctgagcgacctgctgggcttcctggcccactctcgggtgctcgcaggccttgctgctcggggagcagccggttgtgccgccgacagcgtgttcctgaacgtgagcgtgcttgaccctgcggccaggctgagactggaggcccggctgggacacctggtggccgccatcctggagcgggagcagagcctggcagcccacgctctgggctaccagctggcctttgttctggacagcccagccgcctacggagccgttgccccttccgcagccagactgatcgacgccctgtacgccgaattcttaggtggacgggccctgaccgcaccaatggtgcggcgggccctgttctacgccaccgctgtcctgcgggcaccattccttgccggagccccatctgccgagcagcgggagcgtgccagaaggggtctgctgatcaccaccgccctgtgcaccagcgacgttgctgccgccacccacgctgatttacgagctgccctcgcacggaccgaccaccagaagaacctgttctggctgcccgaccacttctctccttgcgccgcgagcctacggttcgacctggccgaaggcggcttcatcctggacgccctcgccatggccacccggagcgacatccccgccgacgtgatggcccaacagacccggggagtggccagtgcactgacccggtgggcccactacaacgccctgatccgggccttcgtgcccgaggccacccaccagtgcagcggccccagccacaacgccgagccccggatcctggtgcccatcacccacaacgctagctacgtggtgacccacactcccttgccccggggaatcggctacaagctgaccggcgtggacgttcggcggcccctgttcatcacctacctgaccgccacctgtgaggggcacgcccgagagatcgagcccaagcggctggtgcggaccgagaatcggcgagacctgggcctggtgggtgccgtgttcctgcggtacacacccgccggcgaggtgatgagcgtgctgctggtggacaccgacgccacccagcagcaattggcccaaggccccgttgccggcacacccaacgtgttcagcagcgacgtgcccagcgtggccctgctgctgttccccaacggcaccgtgatccacctgctggccttcgacaccctgcccatcgccaccatcgccccagggttcctggctgcgagcgctctgggagtggtgatgatcaccgccgccttagccggcatcctgcgggttgtgcggacctgcgtgcccttcctgtggcggcgggag

SEQ ID NO: 11 - RNA, 2259 nt, synthetic construct
  source 1..2259: mol_type = other RNA
atggagccacgacccggcacatccagcagagctgaccccggtcctgaacggcctcctcggcagacccctggcactcaaccagccgcaccacacgcctggggcatgctgaacgacatgcagtggctggccagcagcgacagcgaggaggagaccgaggtgggcatcagcgacgacgacctgcaccgggacagcaccagcgaagccggcagcaccgacaccgagatgttcgaggccggcctgatggacgccgctactccgccagcaaggccaccagcagagcggcaaggtagccccacgcctgctgacgctcagggctcctgtggaggaggccccgtgggtgaggaggaagcagaggccggcggtggtggagacgtgaacactcccgtggcctacctgatcgttggcgtgacagccagcggcagcttcagcaccatccccatcgtgaacgatccacggactcgggtcgaggccgaagcagcagtgagggccggtacagccgtggacttcatctggaccggcaaccctcggaccgcgccaaggagcctgagcctgggcggtcacaccgtccgggctctgtcacctactcctccctggccaggcaccgacgacgaggacgacgatctggccgacgtggactacgtgccacccgctcctcgaagagccccgcggagaggtggtggaggtgctggggcgacccgaggtaccagtcagcccgccgcgactagacccgcacctcctggtgcaccccgcagctccagttctggcggcgctcccctgagagcaggcgttggatctggcagtggcggaggacccgctgttgccgcggtagtcccacgggttgccagcctgcctcctgccgcaggtggaggcagggcccaagccaggagggtgggcgaagacgcagccgcagccgaaggccgaacccctcccgcacggcaacccagggctgcccaggagccacccatcgtgatcagcgactctccgcctcccagtccacggagacccgctggtcctggccctctgagcttcgtgagcagcagctcagcccaggtgagctctgggccgggtggaggaggactgccccaatcaagtggcagagccgcccgaccaagagcagcagtcgctcctcgggtgcggtctccacctagggcagcagctgctcccgtggtgagcgcctcagctgacgcagctggtccagctccacccgccgttcccgtcgacgctcaccgtgcgccgagaagccggatgacccaggcccagaccgacacccaagcccagagcctcggccgtgcaggagcaaccgacgcaagaggcagcggcggtccaggtgcagagggtggatccggacctgcggccagcagcagtgccagcagttctgcggcacctcggagcccactggccccacaaggggtgggcgccaaacgtgcagcaccgcgtcgtgccccagacagcgatagcggagaccggggacacggacctctcgcaccagctagtgccggcgcagcacctcctagcgctagccccagcagccaagccgccgtagccgctgctagctcaagcagcgcatcaagcagtagcgcgagctcaagctccgcctctagctcgagcgcaagctccagcagcgccagcagtagcagcgcctcgagcagctctgccagttcctccgccggaggagctggaggcagcgtggcctctgctagtggcgccggtgagcggagagagaccagcttaggaccgcgtgcggcagcacctcggggacctcggaagtgtgcgcgcaagactcggcacgccgaaggaggccctgaacccggagccagagatcctgcaccaggcctgacccggtacctgcccatcgccggcgtgagcagcgtggtggccctggcaccctacgtgaacaagaccgtgaccggagactgcctgcccgtgctggacatggagaccggccacatcggcgcctacgtggtgctggtggaccagaccggcaacgtggccgacctgctgagagcagccgctccagcctggagtcggcggaccctgctgccagagcacgccagaaactgcgtgcggccgcctgactaccccacaccgcccgcaagcgagtggaacagcctgtggatgacacccgtgggcaacatgctgttcgaccagggcactctggtgggcgcactggactttcacggcctgcggtctcggcacccctggtctcgggaacaaggcgctcctgcgcctgctggtgacgcacctgctggccacggcgag

SEQ ID NO: 12 - RNA, 1461 nt, synthetic construct
  source 1..1461: mol_type = other RNA
atgaacactcccgtggcctacctgatcgtgggtgtgaccgccagcggcagcttcagcaccatccccatcgtgaacgatccccggacgcgcgtcgaagcagaagccgccgtgagagcgggtaccgccgtggacttcatctggaccggcaatcctcggaccgctcctcggagcctgagcctgggcggacacaccgtgagggcccttagccccacccctccttggcccggaaccgacgacgaggacgacgacctggccgacgtggactacgtgcctccggctcctagacgcgctcctagaaggggaggtggcggtgctggagcaactaggggaaccagccagcctgccgctacaagacccgcacctcctggtgcaccgcggagcagcagtagcggtggcgcacctctgcgggcaggcgttggatcaggctccggaggcggaccagctgtggccgccgtggttcctcgggtggccagcctaccacctgcagccggtggaggcagagcgcaagctcgacgggtgggagaggacgcagctgctgccgaaggccggacaccacccgctaggcaaccacgggcagcccaggagcctcccatcgtgatcagcgacagcccgccaccaagtccccggagacctgccggacctggacccctgagcttcgtgagcagcagcagcgcccaggtgtcaagcggtccaggaggcggaggcttaccccagagcagcgggagggctgcaagaccacgcgctgcagtggctccgcgggtgagatctccgccacgtgccgctgcagcccctgtggtgagcgcatccgctgacgctgccggtcctgcacctcctgccgtgcccgttgacgcccatagagctccccggagccggatgacccaggcccagaccgacacccaagctcaaagcctgggtcgtgccggtgcaacagacgccaggggcagtggtggaccaggtgcagagggcgggcctggagttccacggggaaccaacacccctggtgccgctccacacgctgccgaaggggctgctgctggcggaggcagtggaggtggctctccagcccctggactgacccggtacctgcccatcgccggcgtgagcagcgtggtggccctggctccctacgtgaacaagaccgtgaccggcgactgcttacccgtgctggacatggagaccggccacatcggcgcctacgtggtgctggtggaccagaccggcaacgtggccgacctcctgagagccgccgcaccagcttggagccggagaaccctgctgcctgagcacgcccggaactgcgtgcggcctcccgactaccccacaccgcctgccagcgagtggaacagcctgtggatgacgcccgtgggcaacatgctgttcgaccagggcaccctggtgggagccctggacttccacggcctgaggagccggcacccttggagccgggagcaaggcgcaccc

SEQ ID NO: 13 - RNA, 3459 nt, synthetic construct
  source 1..3459: mol_type = other RNA
atgagcgccgagcaacaaggacggggcgcagaagtggccatggccgacgaggacggaggaagactccgggccgcagcagaaaccaccggaggtcctggctcacccgatccagcagacggccctcctccaaccccaaatccggatcgaagaccagctgctcggccaggctttgggtggcacggaggacctgaggagaacgaggacgaagcggacgcctcaggcgaggctgtggacgagcccgctgctgacggtgtggtgagcccacggcaactggccctgctggccagcatggtggacgaggccgtgcggaccatccccagtccacctccagagcgagacggggcccaagaggaggccgccagaagccccagtcctccacgcaccccaagcatgcgggctgactacggcgaggagaacgccggaaggtgggttcggggcccagagaccacctctgccgtgcggggtgcttaccccgaccccatggccagcctaagccctagaccacctgcaccagctgctcgggctcctgcctctgcagcagatcacgctgccggcggaactctgggagccgacgacgaggaagccggtgtgcctgctcgcgcacctggagctgctcctcgaccctctcctccgagggctgaaccagcgccagctcggacaccagccgcaaccgcaggacggctggaaaggcgtcgtgccagagcagccgtggctggcagagacgccacaggccgctttaccgccggacggccaagacgggtggagttggacgcagacgccgctagcggtgctttctacgcccggtaccgagacggctacgttagcggagaaccttggcctggagcaggaccaccgccacctggcagagtgctgtacggtggactgggcgacagccgtcctggcctgtggggagctcccgaagcagaggaagcccgggcaagattcgaagccagcggagctccagctcctgtgtgggcacccgagttaggagacgccgcccagcagtacgccctgatcacccggctgctgtacaccccagacgccgaagccatgggctggctgcagaatccaagagtggctcccggagacgtggccctggaccaggcctgcttccggatttccggcgccgcacggaatagcagcagcttcatctctggcagcgttgcccgagccgtgccacacctgggctacgccatggctgcgggcaggtttggctggggcctggctcacgtggccgctgccgtagcaatgagccggcggtacgacagagcccagaagggcttcctgctgaccagcctgcggagagcctacgctccccttctggctcgggagaacgccgctttaacgggcgcacggactcctgacgacggaggcgacgcgaaccgccacgacggtgacgacgccagagggaagcctcccctccctagcgcttccccagccgacgaacgagctgtgccagcgggttacggcgctgccggagtactggctgcgcttggaagactgagcgccgcacctgcatctgcgcctgctggcgctagagctgaggccggtagagtggccgtggagtgcctggccgcctgcagaggcatcctggaagccctggccgagggctttgacggcgatttagccgccgtgcccggattggcaggagcacgcccagctgcacctccacgacctggtcctgcgggtgctgctgctcctccgcacgcagacgctcccagactacgcgcctggcttcgggagctgcggttcgttcgggacgccctggtgctaatgcgcctgagaggcgatctgcgcgtagcgggaggaagcgaagccgctgtggctgctgttcgggccgtgagcctggtggctggagctctaggccccgcattaccccggagccctagactgctgagcagcgacctgctgttccagaaccagagcttgcggcccttactggctgacaccgtggcagcggctgactcccttgctgccccagctagcgcacccagagaggctgctgacgctccaaggcccgctgcagcacgacccgctgcactcacacggaggcctgccgaaggccctgacccacagggcggttggaggcgacagcctcctggcccctcacatacgcccgcacctagcgctgccgctctggaggcctactgcgctcctagagccgtggccgagctgaccgaccatcccctgtttccggcaccttggagacccgccctcatgttcgaccctagagccctggctagccttgcagcgcggtgtgcagctcctcctccaggaggggctccagctgcctttgggccactccgcgctagtggacccctcagaagggccgctgcctggatgcgtcaggtgcccgatcccgaggacgtgcgggtggtgatcctgtatagtcccctgcctggcgaggatttggctgccggaagagcaggaggcggaccacctcctgagtggagtgccgaacggggaggcctgagctgcctgctggctgccctgggcaaccggctgtgcggacctgccactgccgcttgggccggaaactggactggcgctcccgacgtgtcggcactcggagcgcaaggcgtgctgctgctgtccaccagagacctcgcattcgcaggggccgtggagttccttggcctcttggcgggtgcttgcgaccggcggctgatcgtggtgaacgccgttcgcgcagctgcttggcctgcagcagccccagtggtgagccggcagcacgcctacctggcctgtgaggtgctgccagccgtgcagtgcgccgtaaggtggcccgctgctcgagacctgcggcggactgtgctggccagcggtagggtgtttggacccggcgtgttcgcacgagtggaggcagcacacgctcggctgtaccccgacgctcctccactgcggctttgcaggggcgctaacgtgcggtacagggtgcggaccaggttcggccccgacacactggtgcccatgagtccccgggaataccggcgcgctgtgttgcccgcccttgacggaagagctgccgcttccggagctggagacgccatggcccctggtgcaccagacttctgcgaggacgaggcccacagccatcgggcttgtgcacggtggggtctgggagccccactgagacccgtgtacgtagccctgggcagagacgctgtacgcggaggcccagcagagctgaggggacccagacgggagttttgtgcccgggctctgctggagccagacggcgacgctccacctctggtcctgagagacgacgcagacgctgggcctccacctcagatccggtgggcttctgcagctggacgggcaggaaccgtgctggctgcagccggtggtggcgtggaggttgtcgggactgccgctggtctggctacacctccacgacgcgagcccgtggacatggacgccgagctggaggacgacgacgacggcctgttcggcgag

SEQ ID NO: 14 - RNA, 2061 nt, synthetic construct
  source 1..2061: mol_type = other RNA
atgcgggtgctgtacggtggcctgggcgactcaagacctggcctctggggcgcacctgaggcagaggaagcgcgggctcgttttgaggcatctggtgcccctgcccctgtgtgggccccagagctgggagacgccgcccagcagtacgccctgatcacccggctgctgtacacacccgacgccgaggccatgggctggctgcagaaccctcgggtggctccaggggacgtggccctggaccaggcctgcttcagaataagcggggccgctcggaacagcagcagcttcatcagcggcagcgttgctcgagccgtgcctcacctgggctacgcaatggccgccggcagattcggctggggcctggctcacgtcgctgcggcagtggctatgagccggcggtacgacagggcccagaagggcttcctgttgacaagcctgcgccgcgcatacgctccactgttagcccgcgagaacgccgcactgacgggagcccggactcctgacgacggcggagacgccaacagacacgacggggacgacgcaagaggtaaaccgcccctgcccagtgctgcggcctctcccgctgacgagagagccgtgcctgcaggctacggcgcagctggggtgctggcagcactgggcaggctgtcagccgcccctgctagcgctccagctggcgctagggctgaagcaggcagggtggccgtggagtgcttagccgcctgtcggggcatcctggaggccctggccgaaggctttgacggggacctggctgcggtgcctggcttggccggtgccagacctgcagcacctccccgacctggtccagcaggcgcagcagctcctcctcacgctgacgccccacgcctgagagcctggctgcgggagctgcggttcgtgcgggacgccctggtgctgatgcggctgagaggcgacctgcgtgttgctggcggttccgaggcagctgtggccgcagtgagagccgttagcctggtggccggcgcattaggaccagccctgccacgtagcccgagactgctgagcagcgacctgctgttccagaatcagtctttgggtggagggtccggtggaggacctcctccaggtggagcacctgccgccttcggccctttaagggcgtctggccctctgcgtagagcagccgcctggatgcggcaggtgcccgatcccgaggacgtgcgggtggtgatcctgtacagtcccctgcccggcgaagatctcgcagccggaagagccggaggcggaccaccacctgagtggagcgccgaacgaggtgggctgagctgtcttctcgccgcgctgggcaacagactctgtggacccgctacagccgcctgggctggcaattggacaggtgccccggacgtatccgccctgggcgcacaaggcgtgctgctgctgagcacccgggatctggcattcgccggcgccgtggagtttctgggactgctggcaggtgcctgtgaccggcggctgatcgtggtcaacgcagtcagagccgctgactggcctgcggacggacctgtggtgagccggcagcacgcctacctggcctgcgaggtgctgccagccgtgcagtgtgcagttcggtggcctgccgccagagacctgcggcggaccgtgttagccagcggccgggttttcggtcccggagtgttcgctcgggtggaagcagcccacgcacggctgtaccccgacgccccacctctgcggctgtgcagaggagcaaacgtgcggtaccgggtgcggacacggttcggcccagacaccctggtgcccatgtcaccccgggagtaccggagagccgtgttacctgcccttgacggaagggcagccgcttctggggccggtgacgctatggctcctggcgctcccgacttctgcgaggacgaagcccacagccaccgagcctgcgccaggtggggtctcggtgccccactgagacccgtgtacgtggctctgggacgggacgctgtacggggtgggcccgctgaacttagagggccacggcgggagttctgtgcacgggccctgctggag

SEQ ID NO: 15 - RNA, 2703 nt, synthetic construct
  source 1..2703: mol_type = other RNA
atgcggggtggcggactggtgtgcgccctggtggtcggtgcacttgtggccgctgtggctagcgctgcaccagccgcacccagagccagcggaggtgtggccgcgaccgttgcagccaacggaggcccggcttctcagcctcctcctgtgcccagccccgctaccaccaaggcccgtaagcggaagaccaagaagcctcccaagcggcccgaggctacaccaccacccgacgccaacgcaactgttgctgctggccacgccaccctgagggcccacctgcgggagatcaaggtggagaacgccgacgcccagttctacgtgtgtccacctccaaccggcgccaccgtggtacagttcgagcaacctcgccggtgccccactcgacccgagggccagaactacaccgagggcatcgccgtggtgttcaaggagaacatcgccccatacaagttcaaggccaccatgtactacaaggacgtgaccgtgagccaggtgtggttcggccaccggtacagccagttcatgggcatcttcgaggatcgggccccagtgcccttcgaggaggtgatcgacaagatcaacgccaagggcgtgtgccggagcaccgccaagtacgtgcggaacaacatggagaccaccgccttccaccgggacgaccacgagaccgacatggagctgaagcccgccaaggtggccacacggacaagccggggctggcacaccaccgacctgaagtacaacccaagccgcgtggaggcgttccaccggtacggcaccaccgtgaactgcatcgtggaggaggtggacgcccggagcgtgtacccctacgacgaattcgtgctggccaccggcgacttcgtgtacatgagccccttctacggctaccgggagggcagccacaccgagcacaccagctacgccgccgaccggttcaagcaggtggacggcttctacgcccgggacctgaccactaaagcccgggccacctcaccaaccacccggaacctgctgaccaccccaaagttcaccgtggcctgggactgggtgcccaagcgacccgccgtgtgcaccatgaccaagtggcaggaagtggacgagatgctgcgggccgaatacggcggcagcttccggttcagcagcgacgccatcagcaccaccttcaccaccaacctgacccagtacagcctgagccgggtggatctgggcgactgcatcggcagagacgctcgcgaggccatcgaccggatgttcgcccggaagtacaacgccacccacatcaaggtgggccagccccagtactacctcgccaccggcggcttcctgatcgcctaccagcccttgctgagcaacaccctggccgagctgtacgtgcgggagtacatgcgggagcaggaccggaagccccggaacgccacacccgcccctctgagagaagccccttccgccaacgccagcgtggagcggatcaagaccaccagcagcatcgagttcgcccggctgcagttcacctacaaccacatccagcggcacgtgaacgacatgctgggtcggcctcccgtggcttggtgcgagctgcagaaccacgagctgaccctgtggaacgaggcccggaagctgaaccccaacgccatcgccagcgctactgtgggccggagggtaagcgctcggatgctgggcgacgtgatggccgtgagcacgtgtgtgcccgtggccccagacaacgtgatcgtgcagaacagcatgcgggtgagtagccggcctggaacctgctacagccggcccctggtgtctttccggtacgaggaccagggtcccctgatcgagggccagctgggcgagaacaacgagctgcggctgactcgcgacgctctggagccctgcaccgtgggccacagacggtacttcatcttcggcggcggctacgtgtacttcgaggagtacgcctacagccaccagctgagccgggccgacgtgaccaccgtgagcaccttcatcgacctgaacatcaccatgctggaggaccacgagttcgtgcccctggaggtgtacacccggcacgagatcaaggacagcggcctgctggattacaccgaggtgcagcggcggaaccagctgcacgacctgcggttcgccgacatcgacaccgtgatacgggccgacgcaaacgccgccatgttcgccggcctgtgcgccttcttcgagggcatgggcgacctgggcagagccgtgggcaaggtggtgatgggcgtggtgggcggagtggtaagcgccgtgagcggcgtgagcagcttcatgagcaaccccttcggtgccctggcagtgggcctgctggtgcttgctggcctggtcgctgccttcttcgccttccggtacgtgctacagctgcagcggaaccccatgaaggccctgtaccctctgaccaccaaggagctgaagacctcagaccctggcggcgttggaggagagggtgaggagggagcagagggcggaggcttcgacgaggccaagctggctgaggcccgggagatgatccggtacatggccctggtgagcgccatggagcggaccgagcacaaggccagaaagaagggcaccagcgccctgctgagcagcaaggtgaccaacatggtgctgcggaagcggaacaaggcccggtactcacccctgcacaacgaggacgaggccggcgacgaagacgagctg

SEQ ID NO: 16 - RNA, 2703 nt, synthetic construct
  source 1..2703: mol_type = other RNA
atgcggggtggcggactggtgtgcgccctggtggtcggtgcacttgtggccgctgtggctagcgctgcaccagccgcacccagagccagcggaggtgtggccgcgaccgttgcagccaacggaggcccggcttctcagcctcctcctgtgcccagccccgctaccaccaaggcccgtaagcggaagaccaagaagcctcccaagcggcccgaggctacaccaccacccgacgccaacgcaactgttgctgctggccacgccaccctgagggcccacctgcgggagatcaaggtggagaacgccgacgcccagttctacgtgtgtccacctccaaccggcgccaccgtggtacagttcgagcaacctcgccggtgccccactcgacccgagggccagaactacaccgagggcatcgccgtggtgttcaaggagaacatcgccccatacaagttcaaggccaccatgtactacaaggacgtgaccgtgagccaggtgtggttcggccaccggtacagccagttcatgggcatcttcgaggatcgggccccagtgcccttcgaggaggtgatcgacaagatcaacgccaagggcgtgtgccggagcaccgccaagtacgtgcggaacaacatggagaccaccgccttccaccgggacgaccacgagaccgacatggagctgaagcccgccaaggtggccacacggacaagccggggctggcacaccaccgacctgaagtacaacccaagccgcgtggaggcgttccaccggtacggcaccaccgtgaactgcatcgtggaggaggtggacgcccggagcgtgtacccctacgacgaattcgtgctggccaccggcgacttcgtgtacatgagccccttctacggctaccgggagggcagccacaccgagcacaccagctacgccgccgaccggttcaagcaggtggacggcttctacgcccgggacctgaccactaaagcccgggccacctcaccaaccacccggaacctgctgaccaccccaaagttcaccgtggcctgggactgggtgcccaagcgacccgccgtgtgcaccatgaccaagtggcaggaagtggacgagatgctgcgggccgaatacggcggcagcttccggttcagcagcgacgccatcagcaccaccttcaccaccaacctgacccagtacagcctgagccgggtggatctgggcgactgcatcggcagagacgctcgcgaggccatcgaccggatgttcgcccggaagtacaacgccacccacatcaaggtgggccagccccagtactacctcgccaccggcggcttcctgatcgcctaccagcccttgctgagcaacaccctggccgagctgtacgtgcgggagtacatgcgggagcaggaccggaagccccggaacgccacacccgcccctctgagagaagccccttccgccaacgccagcgtggagcggatcaagaccaccagcagcatcgagttcgcccggctgcagttcacctacaaccacatccagcggcacgtgaacgaccctcccggtcggatcgccgtggcttggtgcgagctgcagaaccacgagctgaccctgtggaacgaggcccggaagctgaaccccaacgccatcgccagcgctactgtgggccggagggtaagcgctcggatgctgggcgacgtgatggccgtgagcacgtgtgtgcccgtggccccagacaacgtgatcgtgcagaacagcatgcgggtgagtagccggcctggaacctgctacagccggcccctggtgtctttccggtacgaggaccagggtcccctgatcgagggccagctgggcgagaacaacgagctgcggctgactcgcgacgctctggagccctgcaccgtgggccacagacggtacttcatcttcggcggcggctacgtgtacttcgaggagtacgcctacagccaccagctgagccgggccgacgtgaccaccgtgagcaccttcatcgacctgaacatcaccatgctggaggaccacgagttcgtgcccctggaggtgtacacccggcacgagatcaaggacagcggcctgctggattacaccgaggtgcagcggcggaaccagctgcacgacctgcggttcgccgacatcgacaccgtgatacgggccgacgcaaacgccgccatgttcgccggcctgtgcgccttcttcgagggcatgggcgacctgggcagagccgtgggcaaggtggtgatgggcgtggtgggcggagtggtaagcgccgtgagcggcgtgagcagcttcatgagcaaccccttcggtgccctggcagtgggcctgctggtgcttgctggcctggtcgctgccttcttcgccttccggtacgtgctacagctgcagcggaaccccatgaaggccctgtaccctctgaccaccaaggagctgaagacctcagaccctggcggcgttggaggagagggtgaggagggagcagagggcggaggcttcgacgaggccaagctggctgaggcccgggagatgatccggtacatggccctggtgagcgccatggagcggaccgagcacaaggccagaaagaagggcaccagcgccctgctgagcagcaaggtgaccaacatggtgctgcggaagcggaacaaggcccggtactcacccctgcacaacgaggacgaggccggcgacgaagacgagctg

SEQ ID NO: 17 - RNA, 2703 nt, synthetic construct
  source 1..2703: mol_type = other RNA
atgcggggtggcggactggtgtgcgccctggtggtcggtgcacttgtggccgctgtggctagcgctgcaccagccgcacccagagccagcggaggtgtggccgcgaccgttgcagccaacggaggcccggcttctcagcctcctcctgtgcccagccccgctaccaccaaggcccgtaagcggaagaccaagaagcctcccaagcggcccgaggctacaccaccacccgacgccaacgcaactgttgctgctggccacgccaccctgagggcccacctgcgggagatcaaggtggagaacgccgacgcccagttctacgtgtgtccacctccaaccggcgccaccgtggtacagttcgagcaacctcgccggtgccccactcgacccgagggccagaactacaccgagggcatcgccgtggtgttcaaggagaacatcgccccatacaagttcaaggccaccatgtactacaaggacgtgaccgtgagccaggtgtggttcggccaccggtacagccagttcatgggcatcttcgaggatcgggccccagtgcccttcgaggaggtgatcgacaagatcaacgccaagggcgtgtgccggagcaccgccaagtacgtgcggaacaacatggagaccaccgccttccaccgggacgaccacgagaccgacatggagctgaagcccgccaaggtggccacacggacaagccggggctggcacaccaccgacctgaagtacaacccaagccgcgtggaggcgttccaccggtacggcaccaccgtgaactgcatcgtggaggaggtggacgcccggagcgtgtacccctacgacgaattcgtgctggccaccggcgacttcgtgtacatgagccccttctacggctaccgggagggcagccacaccgagcacaccagctacgccgccgaccggttcaagcaggtggacggcttctacgcccgggacctgaccactaaagcccgggccacctcaccaaccacccggaacctgctgaccaccccaaagttcaccgtggcctgggactgggtgcccaagcgacccgccgtgtgcaccatgaccaagtggcaggaagtggacgagatgctgcgggccgaatacggcggcagcttccggttcagcagcgacgccatcagcaccaccttcaccaccaacctgacccagtacagcctgagccgggtggatctgggcgactgcatcggcagagacgctcgcgaggccatcgaccggatgttcgcccggaagtacaacgccacccacatcaaggtgggccagccccagtactacctcgccaccggcggcttcctgatcgcctaccagcccttgctgagcaacaccctggccgagctgtacgtgcgggagtacatgcgggagcaggaccggaagccccggaacgccacacccgcccctctgagagaagccccttccgccaacgccagcgtggagcggatcaagaccaccagcagcatcgagttcgcccggctgcagttcacctacaaccacatccagcggcctcccaacgacatgctgggtcggatcgccgtggcttggtgcgagctgcagaaccacgagctgaccctgtggaacgaggcccggaagctgaaccccaacgccatcgccagcgctactgtgggccggagggtaagcgctcggatgctgggcgacgtgatggccgtgagcacgtgtgtgcccgtggccccagacaacgtgatcgtgcagaacagcatgcgggtgagtagccggcctggaacctgctacagccggcccctggtgtctttccggtacgaggaccagggtcccctgatcgagggccagctgggcgagaacaacgagctgcggctgactcgcgacgctctggagccctgcaccgtgggccacagacggtacttcatcttcggcggcggctacgtgtacttcgaggagtacgcctacagccaccagctgagccgggccgacgtgaccaccgtgagcaccttcatcgacctgaacatcaccatgctggaggaccacgagttcgtgcccctggaggtgtacacccggcacgagatcaaggacagcggcctgctggattacaccgaggtgcagcggcggaaccagctgcacgacctgcggttcgccgacatcgacaccgtgatacgggccgacgcaaacgccgccatgttcgccggcctgtgcgccttcttcgagggcatgggcgacctgggcagagccgtgggcaaggtggtgatgggcgtggtgggcggagtggtaagcgccgtgagcggcgtgagcagcttcatgagcaaccccttcggtgccctggcagtgggcctgctggtgcttgctggcctggtcgctgccttcttcgccttccggtacgtgctacagctgcagcggaaccccatgaaggccctgtaccctctgaccaccaaggagctgaagacctcagaccctggcggcgttggaggagagggtgaggagggagcagagggcggaggcttcgacgaggccaagctggctgaggcccgggagatgatccggtacatggccctggtgagcgccatggagcggaccgagcacaaggccagaaagaagggcaccagcgccctgctgagcagcaaggtgaccaacatggtgctgcggaagcggaacaaggcccggtactcacccctgcacaacgaggacgaggccggcgacgaagacgagctg

SEQ ID NO: 18 - RNA, 2703 nt, synthetic construct
  source 1..2703: mol_type = other RNA
atgcggggtggcggactggtgtgcgccctggtggtcggtgcacttgtggccgctgtggctagcgctgcaccagccgcacccagagccagcggaggtgtggccgcgaccgttgcagccaacggaggcccggcttctcagcctcctcctgtgcccagccccgctaccaccaaggcccgtaagcggaagaccaagaagcctcccaagcggcccgaggctacaccaccacccgacgccaacgcaactgttgctgctggccacgccaccctgagggcccacctgcgggagatcaaggtggagaacgccgacgcccagttctacgtgtgtccacctccaaccggcgccaccgtggtacagttcgagcaacctcgccggtgccccactcgacccgagggccagaactacaccgagggcatcgccgtggtgttcaaggagaacatcgccccatacaagttcaaggccaccatgtactacaaggacgtgaccgtgagccaggtgtggttcggccaccggtacagccagttcatgggcatcttcgaggatcgggccccagtgcccttcgaggaggtgatcgacaagatcaacgccaagggcgtgtgccggagcaccgccaagtacgtgcggaacaacatggagaccaccgccttccaccgggacgaccacgagaccgacatggagctgaagcccgccaaggtggccacacggacaagccggggctggcacaccaccgacctgaagtacaacccaagccgcgtggaggcgttccaccggtacggcaccaccgtgaactgcatcgtggaggaggtggacgcccggagcgtgtacccctacgacgaattcgtgctggccaccggcgacttcgtgtacatgagccccttctacggctaccgggagggcagccacaccgagcacaccagctacgccgccgaccggttcaagcaggtggacggcttctacgcccgggacctgaccactaaagcccgggccacctcaccaaccacccggaacctgctgaccaccccaaagttcaccgtggcctgggactgggtgcccaagcgacccgccgtgtgcaccatgaccaagtggcaggaagtggacgagatgctgcgggccgaatacggcggcagcttccggttcagcagcgacgccatcagcaccaccttcaccaccaacctgacccagtacagcctgagccgggtggatctgggcgactgcatcggcagagacgctcgcgaggccatcgaccggatgttcgcccggaagtacaacgccacccacatcaaggtgggccagccccagtactacctcgccaccggcggcttcctgatcgcctaccagcccttgctgagcaacaccctggccgagctgtacgtgcgggagtacatgcgggagcaggaccggaagccccggaacgccacacccgcccctctgagagaagccccttccgccaacgccagcgtggagcggatcaagaccaccagcagcatcgagttcgcccggctgcagttcacctacaaccctccccagcggcacgtgaacgacatgctgggtcggatcgccgtggcttggtgcgagctgcagaaccacgagctgaccctgtggaacgaggcccggaagctgaaccccaacgccatcgccagcgctactgtgggccggagggtaagcgctcggatgctgggcgacgtgatggccgtgagcacgtgtgtgcccgtggccccagacaacgtgatcgtgcagaacagcatgcgggtgagtagccggcctggaacctgctacagccggcccctggtgtctttccggtacgaggaccagggtcccctgatcgagggccagctgggcgagaacaacgagctgcggctgactcgcgacgctctggagccctgcaccgtgggccacagacggtacttcatcttcggcggcggctacgtgtacttcgaggagtacgcctacagccaccagctgagccgggccgacgtgaccaccgtgagcaccttcatcgacctgaacatcaccatgctggaggaccacgagttcgtgcccctggaggtgtacacccggcacgagatcaaggacagcggcctgctggattacaccgaggtgcagcggcggaaccagctgcacgacctgcggttcgccgacatcgacaccgtgatacgggccgacgcaaacgccgccatgttcgccggcctgtgcgccttcttcgagggcatgggcgacctgggcagagccgtgggcaaggtggtgatgggcgtggtgggcggagtggtaagcgccgtgagcggcgtgagcagcttcatgagcaaccccttcggtgccctggcagtgggcctgctggtgcttgctggcctggtcgctgccttcttcgccttccggtacgtgctacagctgcagcggaaccccatgaaggccctgtaccctctgaccaccaaggagctgaagacctcagaccctggcggcgttggaggagagggtgaggagggagcagagggcggaggcttcgacgaggccaagctggctgaggcccgggagatgatccggtacatggccctggtgagcgccatggagcggaccgagcacaaggccagaaagaagggcaccagcgccctgctgagcagcaaggtgaccaacatggtgctgcggaagcggaacaaggcccggtactcacccctgcacaacgaggacgaggccggcgacgaagacgagctg

SEQ ID NO: 19 - RNA, 2394 nt, synthetic construct
  source 1..2394: mol_type = other RNA
atgcggggtggcggactggtgtgcgccctggtggtcggtgcacttgtggccgctgtggctagcgctgcaccagccgcacccagagccagcggaggtgtggccgcgaccgttgcagccaacggaggcccggcttctcagcctcctcctgtgcccagccccgctaccaccaaggcccgtaagcggaagaccaagaagcctcccaagcggcccgaggctacaccaccacccgacgccaacgcaactgttgctgctggccacgccaccctgagggcccacctgcgggagatcaaggtggagaacgccgacgcccagttctacgtgtgtccacctccaaccggcgccaccgtggtacagttcgagcaacctcgccggtgccccactcgacccgagggccagaactacaccgagggcatcgccgtggtgttcaaggagaacatcgccccatacaagttcaaggccaccatgtactacaaggacgtgaccgtgagccaggtgtggttcggccaccggtacagccagttcatgggcatcttcgaggatcgggccccagtgcccttcgaggaggtgatcgacaagatcaacgccaagggcgtgtgccggagcaccgccaagtacgtgcggaacaacatggagaccaccgccttccaccgggacgaccacgagaccgacatggagctgaagcccgccaaggtggccacacggacaagccggggctggcacaccaccgacctgaagtacaacccaagccgcgtggaggcgttccaccggtacggcaccaccgtgaactgcatcgtggaggaggtggacgcccggagcgtgtacccctacgacgaattcgtgctggccaccggcgacttcgtgtacatgagccccttctacggctaccgggagggcagccacaccgagcacaccagctacgccgccgaccggttcaagcaggtggacggcttctacgcccgggacctgaccactaaagcccgggccacctcaccaaccacccggaacctgctgaccaccccaaagttcaccgtggcctgggactgggtgcccaagcgacccgccgtgtgcaccatgaccaagtggcaggaagtggacgagatgctgcgggccgaatacggcggcagcttccggttcagcagcgacgccatcagcaccaccttcaccaccaacctgacccagtacagcctgagccgggtggatctgggcgactgcatcggcagagacgctcgcgaggccatcgaccggatgttcgcccggaagtacaacgccacccacatcaaggtgggccagccccagtactacctcgccaccggcggcttcctgatcgcctaccagcccttgctgagcaacaccctggccgagctgtacgtgcgggagtacatgcgggagcaggaccggaagccccggaacgccacacccgcccctctgagagaagccccttccgccaacgccagcgtggagcggatcaagaccaccagcagcatcgagttcgcccggctgcagttcacctacaaccacatccagcggcacgtgaacgacatgctgggtcggatcgccgtggcttggtgcgagctgcagaaccacgagctgaccctgtggaacgaggcccggaagctgaaccccaacgccatcgccagcgctactgtgggccggagggtaagcgctcggatgctgggcgacgtgatggccgtgagcacgtgtgtgcccgtggccccagacaacgtgatcgtgcagaacagcatgcgggtgagtagccggcctggaacctgctacagccggcccctggtgtctttccggtacgaggaccagggtcccctgatcgagggccagctgggcgagaacaacgagctgcggctgactcgcgacgctctggagccctgcaccgtgggccacagacggtacttcatcttcggcggcggctacgtgtacttcgaggagtacgcctacagccaccagctgagccgggccgacgtgaccaccgtgagcaccttcatcgacctgaacatcaccatgctggaggaccacgagttcgtgcccctggaggtgtacacccggcacgagatcaaggacagcggcctgctggattacaccgaggtgcagcggcggaaccagctgcacgacctgcggttcgccgacatcgacaccgtgatacgggccgacgcaaacgccgccatgttcgccggcctgtgcgccttcttcgagggcatgggcgacctgggcagagccgtgggcaaggtggtgatgggcgtggtgggcggagtggtaagcgccgtgagcggcgtgagcagcttcatgagcaaccccttcggtgccctggcagtgggcctgctggtgcttgctggcctggtcgctgccttcttcgccttccggtacgtgctacagctgcagcggaac

SEQ ID NO: 20 - RNA, 2394 nt, synthetic construct
  source 1..2394: mol_type = other RNA
atgcggggtggcggactggtgtgcgccctggtggtcggtgcacttgtggccgctgtggctagcgctgcaccagccgcacccagagccagcggaggtgtggccgcgaccgttgcagccaacggaggcccggcttctcagcctcctcctgtgcccagccccgctaccaccaaggcccgtaagcggaagaccaagaagcctcccaagcggcccgaggctacaccaccacccgacgccaacgcaactgttgctgctggccacgccaccctgagggcccacctgcgggagatcaaggtggagaacgccgacgcccagttctacgtgtgtccacctccaaccggcgccaccgtggtacagttcgagcaacctcgccggtgccccactcgacccgagggccagaactacaccgagggcatcgccgtggtgttcaaggagaacatcgccccatacaagttcaaggccaccatgtactacaaggacgtgaccgtgagccaggtgtggttcggccaccggtacagccagttcatgggcatcttcgaggatcgggccccagtgcccttcgaggaggtgatcgacaagatcaacgccaagggcgtgtgccggagcaccgccaagtacgtgcggaacaacatggagaccaccgccttccaccgggacgaccacgagaccgacatggagctgaagcccgccaaggtggccacacggacaagccggggctggcacaccaccgacctgaagtacaacccaagccgcgtggaggcgttccaccggtacggcaccaccgtgaactgcatcgtggaggaggtggacgcccggagcgtgtacccctacgacgaattcgtgctggccaccggcgacttcgtgtacatgagccccttctacggctaccgggagggcagccacaccgagcacaccagctacgccgccgaccggttcaagcaggtggacggcttctacgcccgggacctgaccactaaagcccgggccacctcaccaaccacccggaacctgctgaccaccccaaagttcaccgtggcctgggactgggtgcccaagcgacccgccgtgtgcaccatgaccaagtggcaggaagtggacgagatgctgcgggccgaatacggcggcagcttccggttcagcagcgacgccatcagcaccaccttcaccaccaacctgacccagtacagcctgagccgggtggatctgggcgactgcatcggcagagacgctcgcgaggccatcgaccggatgttcgcccggaagtacaacgccacccacatcaaggtgggccagccccagtactacctcgccaccggcggcttcctgatcgcctaccagcccttgctgagcaacaccctggccgagctgtacgtgcgggagtacatgcgggagcaggaccggaagccccggaacgccacacccgcccctctgagagaagccccttccgccaacgccagcgtggagcggatcaagaccaccagcagcatcgagttcgcccggctgcagttcacctacaaccacatccagcggcacgtgaacgaccctcccggtcggatcgccgtggcttggtgcgagctgcagaaccacgagctgaccctgtggaacgaggcccggaagctgaaccccaacgccatcgccagcgctactgtgggccggagggtaagcgctcggatgctgggcgacgtgatggccgtgagcacgtgtgtgcccgtggccccagacaacgtgatcgtgcagaacagcatgcgggtgagtagccggcctggaacctgctacagccggcccctggtgtctttccggtacgaggaccagggtcccctgatcgagggccagctgggcgagaacaacgagctgcggctgactcgcgacgctctggagccctgcaccgtgggccacagacggtacttcatcttcggcggcggctacgtgtacttcgaggagtacgcctacagccaccagctgagccgggccgacgtgaccaccgtgagcaccttcatcgacctgaacatcaccatgctggaggaccacgagttcgtgcccctggaggtgtacacccggcacgagatcaaggacagcggcctgctggattacaccgaggtgcagcggcggaaccagctgcacgacctgcggttcgccgacatcgacaccgtgatacgggccgacgcaaacgccgccatgttcgccggcctgtgcgccttcttcgagggcatgggcgacctgggcagagccgtgggcaaggtggtgatgggcgtggtgggcggagtggtaagcgccgtgagcggcgtgagcagcttcatgagcaaccccttcggtgccctggcagtgggcctgctggtgcttgctggcctggtcgctgccttcttcgccttccggtacgtgctacagctgcagcggaac

SEQ ID NO: 21 - RNA, 2394 nt, synthetic construct
  source 1..2394: mol_type = other RNA
atgcggggtggcggactggtgtgcgccctggtggtcggtgcacttgtggccgctgtggctagcgctgcaccagccgcacccagagccagcggaggtgtggccgcgaccgttgcagccaacggaggcccggcttctcagcctcctcctgtgcccagccccgctaccaccaaggcccgtaagcggaagaccaagaagcctcccaagcggcccgaggctacaccaccacccgacgccaacgcaactgttgctgctggccacgccaccctgagggcccacctgcgggagatcaaggtggagaacgccgacgcccagttctacgtgtgtccacctccaaccggcgccaccgtggtacagttcgagcaacctcgccggtgccccactcgacccgagggccagaactacaccgagggcatcgccgtggtgttcaaggagaacatcgccccatacaagttcaaggccaccatgtactacaaggacgtgaccgtgagccaggtgtggttcggccaccggtacagccagttcatgggcatcttcgaggatcgggccccagtgcccttcgaggaggtgatcgacaagatcaacgccaagggcgtgtgccggagcaccgccaagtacgtgcggaacaacatggagaccaccgccttccaccgggacgaccacgagaccgacatggagctgaagcccgccaaggtggccacacggacaagccggggctggcacaccaccgacctgaagtacaacccaagccgcgtggaggcgttccaccggtacggcaccaccgtgaactgcatcgtggaggaggtggacgcccggagcgtgtacccctacgacgaattcgtgctggccaccggcgacttcgtgtacatgagccccttctacggctaccgggagggcagccacaccgagcacaccagctacgccgccgaccggttcaagcaggtggacggcttctacgcccgggacctgaccactaaagcccgggccacctcaccaaccacccggaacctgctgaccaccccaaagttcaccgtggcctgggactgggtgcccaagcgacccgccgtgtgcaccatgaccaagtggcaggaagtggacgagatgctgcgggccgaatacggcggcagcttccggttcagcagcgacgccatcagcaccaccttcaccaccaacctgacccagtacagcctgagccgggtggatctgggcgactgcatcggcagagacgctcgcgaggccatcgaccggatgttcgcccggaagtacaacgccacccacatcaaggtgggccagccccagtactacctcgccaccggcggcttcctgatcgcctaccagcccttgctgagcaacaccctggccgagctgtacgtgcgggagtacatgcgggagcaggaccggaagccccggaacgccacacccgcccctctgagagaagccccttccgccaacgccagcgtggagcggatcaagaccaccagcagcatcgagttcgcccggctgcagttcacctacaaccctccccagcggcacgtgaacgacatgctgggtcggatcgccgtggcttggtgcgagctgcagaaccacgagctgaccctgtggaacgaggcccggaagctgaaccccaacgccatcgccagcgctactgtgggccggagggtaagcgctcggatgctgggcgacgtgatggccgtgagcacgtgtgtgcccgtggccccagacaacgtgatcgtgcagaacagcatgcgggtgagtagccggcctggaacctgctacagccggcccctggtgtctttccggtacgaggaccagggtcccctgatcgagggccagctgggcgagaacaacgagctgcggctgactcgcgacgctctggagccctgcaccgtgggccacagacggtacttcatcttcggcggcggctacgtgtacttcgaggagtacgcctacagccaccagctgagccgggccgacgtgaccaccgtgagcaccttcatcgacctgaacatcaccatgctggaggaccacgagttcgtgcccctggaggtgtacacccggcacgagatcaaggacagcggcctgctggattacaccgaggtgcagcggcggaaccagctgcacgacctgcggttcgccgacatcgacaccgtgatacgggccgacgcaaacgccgccatgttcgccggcctgtgcgccttcttcgagggcatgggcgacctgggcagagccgtgggcaaggtggtgatgggcgtggtgggcggagtggtaagcgccgtgagcggcgtgagcagcttcatgagcaaccccttcggtgccctggcagtgggcctgctggtgcttgctggcctggtcgctgccttcttcgccttccggtacgtgctacagctgcagcggaac

SEQ ID NO: 22 - RNA, 2394 nt, synthetic construct
  source 1..2394: mol_type = other RNA
atgcggggtggcggactggtgtgcgccctggtggtcggtgcacttgtggccgctgtggctagcgctgcaccagccgcacccagagccagcggaggtgtggccgcgaccgttgcagccaacggaggcccggcttctcagcctcctcctgtgcccagccccgctaccaccaaggcccgtaagcggaagaccaagaagcctcccaagcggcccgaggctacaccaccacccgacgccaacgcaactgttgctgctggccacgccaccctgagggcccacctgcgggagatcaaggtggagaacgccgacgcccagttctacgtgtgtccacctccaaccggcgccaccgtggtacagttcgagcaacctcgccggtgccccactcgacccgagggccagaactacaccgagggcatcgccgtggtgttcaaggagaacatcgccccatacaagttcaaggccaccatgtactacaaggacgtgaccgtgagccaggtgtggttcggccaccggtacagccagttcatgggcatcttcgaggatcgggccccagtgcccttcgaggaggtgatcgacaagatcaacgccaagggcgtgtgccggagcaccgccaagtacgtgcggaacaacatggagaccaccgccttccaccgggacgaccacgagaccgacatggagctgaagcccgccaaggtggccacacggacaagccggggctggcacaccaccgacctgaagtacaacccaagccgcgtggaggcgttccaccggtacggcaccaccgtgaactgcatcgtggaggaggtggacgcccggagcgtgtacccctacgacgaattcgtgctggccaccggcgacttcgtgtacatgagccccttctacggctaccgggagggcagccacaccgagcacaccagctacgccgccgaccggttcaagcaggtggacggcttctacgcccgggacctgaccactaaagcccgggccacctcaccaaccacccggaacctgctgaccaccccaaagttcaccgtggcctgggactgggtgcccaagcgacccgccgtgtgcaccatgaccaagtggcaggaagtggacgagatgctgcgggccgaatacggcggcagcttccggttcagcagcgacgccatcagcaccaccttcaccaccaacctgacccagtacagcctgagccgggtggatctgggcgactgcatcggcagagacgctcgcgaggccatcgaccggatgttcgcccggaagtacaacgccacccacatcaaggtgggccagccccagtactacctcgccaccggcggcttcctgatcgcctaccagcccttgctgagcaacaccctggccgagctgtacgtgcgggagtacatgcgggagcaggaccggaagccccggaacgccacacccgcccctctgagagaagccccttccgccaacgccagcgtggagcggatcaagaccaccagcagcatcgagttcgcccggctgcagttcacctacaaccctccccagcggcctcccaacgacatgctgggtcggatcgccgtggcttggtgcgagctgcagaaccacgagctgaccctgtggaacgaggcccggaagctgaaccccaacgccatcgccagcgctactgtgggccggagggtaagcgctcggatgctgggcgacgtgatggccgtgagcacgtgtgtgcccgtggccccagacaacgtgatcgtgcagaacagcatgcgggtgagtagccggcctggaacctgctacagccggcccctggtgtctttccggtacgaggaccagggtcccctgatcgagggccagctgggcgagaacaacgagctgcggctgactcgcgacgctctggagccctgcaccgtgggccacagacggtacttcatcttcggcggcggctacgtgtacttcgaggagtacgcctacagccaccagctgagccgggccgacgtgaccaccgtgagcaccttcatcgacctgaacatcaccatgctggaggaccacgagttcgtgcccctggaggtgtacacccggcacgagatcaaggacagcggcctgctggattacaccgaggtgcagcggcggaaccagctgcacgacctgcggttcgccgacatcgacaccgtgatacgggccgacgcaaacgccgccatgttcgccggcctgtgcgccttcttcgagggcatgggcgacctgggcagagccgtgggcaaggtggtgatgggcgtggtgggcggagtggtaagcgccgtgagcggcgtgagcagcttcatgagcaaccccttcggtgccctggcagtgggcctgctggtgcttgctggcctggtcgctgccttcttcgccttccggtacgtgctacagctgcagcggaac

SEQ ID NO: 23 - RNA, 1116 nt, synthetic construct
  source 1..1116: mol_type = other RNA
atgcccggccggtctctgcagggcctggccatcctgggcctgtgggtgtgcgccaccggcttagtggtgcgcggccctaccgtgagcctggtgagcgactcactggtggacgctggcgccgttggtccacagggcttcgtggaggaggacctgcgggtgttcggcgagctgcacttcgtgggcgcacaggtgccccacaccaactactacgacggcatcatcgagctgttccactatcccctgggcaaccactgccctcgggtggtgcacgtggtgaccctgaccgcctgcccacggagacccgctgtggccttcaccctgtgccggagcacccaccacgcccacagcccagcctaccccaccttagagctgggccttgccaggcagcctctgctgagagtgcggaccgccactcgggactacgccggcctgtacgtgctgagggtgtgggtgggcagcgccaccaacgccagccggttcgtgctgggagtggccctgagcgccaacggcaccttcgtgtacaacggcagcgactacggctcttgtgaccccgcccagctgccattcagcgccccaaggctgggacccagcagcgtgtacaccccaggagcctccagaccaacaccaccacggaccaccacccctccaagcagtccccgggatcccaccccagctccaggcgatacagggactcccgctccagcaagcggcgagatcgcccctcccaacagcacccggagcgctagcgaaagcaggcaccggctgaccgtggcccaggtgatccagatcgccatccccgccagcattatcgcctttgtgttcctgggcagctgcatctgcttcatacatcggtgccagcggcggtatagacgcccgcgaggacagatctacaaccctggcggcgtgtcctgcgctgtgaacgaggccgccatggccagactgggcgccgagctgaggagccaccccaacaccccaccaaagcctcggcggcgaagctcaagcagcacaaccatgccaagcctgaccagcatcgccgaggaaagcgaacccgggccagtggtgctgctgagcgtgtccccacgaccacggagtggtcccactgcacctcaggaggtg

SEQ ID NO: 24 - RNA, 762 nt, synthetic construct
  source 1..762: mol_type = other RNA
atgcccggccggtctctgcagggcctggccatcctgggcctgtgggtgtgcgccaccggcttagtggtgcgcggccctaccgtgagcctggtgagcgactcactggtggacgctggcgccgttggtccacagggcttcgtggaggaggacctgcgggtgttcggcgagctgcacttcgtgggcgcacaggtgccccacaccaactactacgacggcatcatcgagctgttccactatcccctgggcaaccactgccctcgggtggtgcacgtggtgaccctgaccgcctgcccacggagacccgctgtggccttcaccctgtgccggagcacccaccacgcccacagcccagcctaccccaccttagagctgggccttgccaggcagcctctgctgagagtgcggaccgccactcgggactacgccggcctgtacgtgctgagggtgtgggtgggcagcgccaccaacgccagccggttcgtgctgggagtggccctgagcgccaacggcaccttcgtgtacaacggcagcgactacggctcttgtgaccccgcccagctgccattcagcgccccaaggctgggacccagcagcgtgtacaccccaggagcctccagaccaacaccaccacggaccaccacccctccaagcagtccccgggatcccaccccagctccaggcgatacagggactcccgctccagcaagcggcgagatcgcccctcccaacagcacccggagcgctagcgaaagcaggcaccgg

SEQ ID NO: 25 - RNA, 1302 nt, synthetic construct
  source 1..1302: mol_type = other RNA
atggccagaggagccggcctggtgttcttcgtgggcgtgtgggtggtgtcctgcctggccgcagctcccagaaccagctggaagcgggtgaccagcggcgaggacgtggtgttactgccagctccagccgagcggactcgggcccacaagctgctttgggcagccgagccactggacgcctgcgggccattacggcctagctgggtggctctgtggccacctagacgggtgctggagaccgtggtggacgcagcctgcatgcgggcaccagagcccctggccatcgcctactctccacccttcccagccggcgacgaaggcctgtacagcgagctggcttggagagaccgggtggccgtggtgaacgagagcctggtgatctacggcgccctggagaccgacagcggcctgtacaccctgagcgtggtgggcctgagcgacgaggcccggcaagtggccagcgtggtgctggtggttgagcccgcccctgtgccaacgcccactcccgacgactacgacgaggaggacgacgcaggcgtgagcgagcggaccccagtgagcgtgcctccacccactcctcctcggagacctcccgtggctccacctacccatccccgggtgatccccgaggtgagccacgtgcggggcgtgaccgtgcacatggagacacccgaggccatcctgttcgcccctggcgagaccttcggcaccaacgtgagcatccacgccatagcccacgacgacggcccttacgccatggacgtcgtgtggatgcggttcgacgtgcccagcagctgcgccgagatgcggatctacgaggcctgcctgtaccatccccagctgcccgagtgcctgagccccgctgacgcaccctgcgccgtgagcagctgggcctaccggctggctgtgcggagctacgccgggtgtagccggacaacgcctccgccacggtgcttcgccgaggcccggatggaacctgttcccggcctggcctggcttgctagcaccgtgaacctggagttccagcacgccagcccacaacacgccggcctgtacctgtgcgtggtgtacgtggacgaccacatccacgcctggggccacatgaccatcagcaccgccgcccagtaccggaacgccgtggtggagcagcatctgccccagaggcagcccgagccagtggagcccacgagaccacacgtgcgggcacctcaccctgctcccagcgcacgtgggccactgcggctgggagctgtgctgggcgccgctctgctgctggcagccctgggactgagcgcctgggcc

SEQ ID NO: 26 - RNA, 849 nt, synthetic construct
  source 1..849: mol_type = other RNA
atgcccggccggtctctgcagggcctggccatcctgggcctgtgggtgtgcgccaccggcttagtggtgcgcggccctaccgtgagcctggtgagcgactcactggtggacgctggcgccgttggtccacagggcttcgtggaggaggacctgcgggtgttcggcgagctgcacttcgtgggcgcacaggtgccccacaccaactactacgacggcatcatcgagctgttccactatcccctgggcaaccactgccctcgggtggtgcacgtggtgaccctgaccgcctgcccacggagacccgctgtggccttcaccctgtgccggagcacccaccacgcccacagcccagcctaccccaccttagagctgggccttgccaggcagcctctgctgagagtgcggaccgccactcgggactacgccggcctgtacgtgctgagggtgtgggtgggcagcgccaccaacgccagccggttcgtgctgggagtggccctgagcgccaacggcaccttcgtgtacaacggcagcgactacggctcttgtgaccccgcccagctgccattcagcgccccaaggctgggacccagcagcgtgtacaccccaggagcctccagaccaacaccaccacggaccaccacccctccaagcagtccccgggatcccaccccagctccaggcgatacagggactcccgctccagcaagcggcgagatcgcccctcccaacagcacccggagcgctagcgaaagcaggcaccggctgaccgtggcccaggtgatccagatcgccatccccgccagcattatcgcctttgtgttcctgggcagctgcatctgcttcatacat

SEQ ID NO: 27 - RNA, 1407 nt, synthetic construct
  source 1..1407: mol_type = other RNA
atggcacttggacgcgtcggtctggctgtcggcctgtggggcctgctgtgggtgggcgtggtggtggtgctggctaacgccagccccggacgtaccatcaccgtggggcccagaggcaacgccagtaacgccgcgccatcagccagcccgagaaacgctagtgctccccggaccacgccaactccaccacagccccggaaggccaccaagagcaaggccagcaccgccaagcccgctccacctcccaagaccggcccacccaagaccagcagcgagcccgtgcggtgcaaccggcacgatcctctggcccggtacggctcacgggtgcagatccggtgccggttccccaacagcaccaggaccgagagccggctgcagatctggcggtacgccaccgccacagacgccgagatcggcaccgccccaagcctggaggaggtgatggtgaacgtgtctgctccacctggcggccagctggtgtacgacagcgcacccaaccggaccgatccccacgtgatctgggcagaaggcgctggtcctggcgctagccctcgactgtacagcgtggtcggcccactgggcaggcagcggctgatcatcgaggagctgaccctggagacccagggcatgtactactgggtgtggggccggacagatcggcctagcgcctacgggacctgggtgcgggttcgcgtgttccggccacctagcctgaccatccatccccacgccgtgctggagggccagcccttcaaagccacctgtaccgccgccacctactacccaggcaaccgggccgagttcgtgtggttcgaggacggacggcgcgtgttcgaccccgcccagatccacacccagacccaggagaaccccgacggcttcagcaccgtgagcacggtgaccagcgctgccgttggaggccaaggccctcccagaaccttcacctgccagctgacctggcaccgggacagcgtgagcttttctcgccggaacgccagcggaactgccagcgtgctgccacggcccaccatcaccatggagttcaccggcgaccacgccgtgtgcacagccggctgcgtacccgagggcgtgaccttcgcctggttcctgggcgacgacagcagccccgccgagaaggtggccgtggccagccagaccagctgtggaagacccggaaccgccaccatccggagcaccctgcccgtgagctacgagcagaccgagtacatctgtcggctggccggctaccctgacggcatccccgtgttggagcaccacggcagccaccagcctcctcctcgggatcccaccgagcggcaggtgatcagggccgtggagggtgcaggcatcggcgtggccgtgctggtggccgtagtgctagccggcaccgccgtggtatacctgacc

SEQ ID NO: 28 - RNA, 1407 nt, synthetic construct
  source 1..1407: mol_type = other RNA
atggcacttggacgcgtcggtctggctgtcggcctgtggggcctgctgtgggtgggcgtggtggtggtgctggctaacgccagccccggacgtaccatcaccgtggggcccagaggcaacgccagtaacgccgcgccatcagccagcccgagaaacgctagtgctccccggaccacgccaactccaccacagccccggaaggccaccaagagcaaggccagcaccgccaagcccgctccacctcccaagaccggcccacccaagaccagcagcgagcccgtgcggtgcaaccggcacgatcctctggcccggtacggctcacgggtgcagatccggtgccggttccccaacagcaccaggaccgagagccggctgcagatctggcggtacgccaccgccacagacgccgagatcggcaccgccccaagcctggaggaggtgatggtgaacgtgtctgctccacctggcggccagctggtgtacgacagcgcacccaaccggaccgatccccacgtgatctgggcagaaggcgctggtcctggcgctagccctcgactgtacagcgtggtcggcccactgggcaggcagcggctgatcatcgaggagctgaccctggagacccagggcatgtactactgggtgtggggccggacagatcggcctagcgcctacgggacctgggtgcgggttcgcgtgttccggccacctagcctgaccatccatccccacgccgtgctggagggccagcccttcaaagccacctgtaccgccgccacctactacccaggcaaccgggccgagttcgtgtggttcgaggacggacggcgcgtgttcgaccccgcccagatccacacccagacccaggagaaccccgacggcttcagcaccgtgagcacggtgaccagcgctgccgttggaggccaaggccctcccagaaccttcacctgccagctgacctggcaccgggacagcgtgagcgcttctcgccggaacgccagcggaactgccagcgtgctgccacggcccaccatcaccatggagttcaccggcgaccacgccgtgtgcacagccggctgcgtacccgagggcgtgaccttcgcctggttcctgggcgacgacagcagccccgccgagaaggtggccgtggccagccagaccagctgtggaagacccggaaccgccaccatccggagcaccctgcccgtgagctacgagcagaccgagtacatctgtcggctggccggctaccctgacggcatccccgtgttggagcaccacggcagccaccagcctcctcctcgggatcccaccgagcggcaggtgatcagggccgtggagggtgcaggcatcggcgtggccgtgctggtggccgtagtgctagccggcaccgccgtggtatacctgacc

SEQ ID NO: 29 - RNA, 1089 nt, synthetic construct
  source 1..1089: mol_type = other RNA
atgggtcggctgaccagcggagttggcaccgccgcactcctggtggtggccgtaggcctgcgggtggtgtgcgccaagtacgccctggccgacccatccctgaagatggccgaccctaaccggttccggggcaagaatctgcccgtgctggatcagctgaccgatcctcctggcgtgaagcgggtgtaccacatccagcccagcctggaggatcccttccagccaccctccatccccatcaccgtttactacgccgtgctggagagagcctgccgcagcgtgctgctgcacgctccatccgaggcgccccagatcgtgcggggcgcaagcgacgaggcccggaagcacacctacaacctgaccatcgcctggtaccggatgggcgacaactgcgccatccctatcaccgtgatggagtacaccgagtgcccctacaacaagagcctgggagtgtgccccatccggacccagcctcggtggtcctactacgacagcttcagcgccgtgtccgaggacaacctgggcttcctgatgcacgcccctgccttcgagaccgccggcacctacctgcggctggtgaagatcaacgactggaccgagatcacccagttcatcctggagcaccgggcaagggccagctgcaagtacgcgctgcctctgcggatccctcccgctgcttgcctgaccagcaaggcctaccagcagggcgtgaccgtggacagcatcggcatgctgcccaggttcatccccgagaaccagcgcaccgtggccctgtacagcctgaagatcgcaggctggcacggacccaagcctccttacacctccaccctgctgccacccgagctgagcgacaccaccaacgccacccagcccgagctggtgcccgaggaccccgaggactccgccctgctggaggatccggccggcaccgtaagctcccagatcccacccaactggcacatccccagcatccaggacgtggccccacatcacgcgcctgccgctccaagcaaccccggcctgatcattggagctctggccgggagcactctggcggtgctggtgatcggcggcatcgccttctgggtg

SEQ ID NO: 30 - RNA, 1440 nt, synthetic construct
  source 1..1440: mol_type = other RNA
atggcccttggacgggtaggcctagccgtgggcctgtggggcctactgtgggtgggtgtggtcgtggtgctggccaatgcctcccccggacgcacgataacggtgggcccgcgaggcaacgcgagcaatgctgccccctccgcgtccccgcggaacgcatccgccccccgaaccacacccacgcccccacaaccccgcaaagcgacgaaatccaaggcctccaccgccaaaccggctccgccccccaagaccggacccccgaagacatcctcggagcccgtgcgatgcaaccgccacgacccgctggcccggtacggctcgcgggtgcaaatccgatgccggtttcccaactccacgaggactgagtcccgtctccagatctggcgttatgccacggcgacggacgccgaaatcggaacagcgcctagcttagaagaggtgatggtgaacgtgtcggccccgcccgggggccaactggtgtatgacagtgcccccaaccgaacggacccgcatgtaatctgggcggagggcgccggcccgggcgccagcccgcgcctgtactcggttgtcggcccgctgggtcggcagcggctcatcatcgaagagttaaccctggagacacagggcatgtactattgggtgtggggccggacggaccgcccgtccgcctacgggacctgggtccgcgttcgagtatttcgccctccgtcgctgaccatccacccccacgcggtgctggagggccagccgtttaaggcgacgtgcacggccgcaacctactacccgggcaaccgcgcggagttcgtctggtttgaggacggtcgccgcgtattcgatccggcacagatacacacgcagacgcaggagaaccccgacggcttttccaccgtctccaccgtgacctccgcggccgtcggcgggcagggcccccctcgcaccttcacctgccagctgacgtggcaccgcgactccgtgtcgttctctcggcgcaacgccagcggcacggcctcggttctgccgcggccgaccattaccatggagtttacaggcgaccatgcggtctgcacggccggctgtgtgcccgagggggtcacgtttgcttggttcctgggggatgactcctcgccggcggaaaaggtggccgtcgcgtcccagacatcgtgcgggcgccccggcaccgccacgatccgctccaccctgccggtctcgtacgagcagaccgagtacatctgtagactggcgggatacccggacggaattccggtcctagagcaccacggaagccaccagcccccgccgcgggacccaaccgagcggcaggtgatccgggcggtggagggggcggggatcggagtggctgtccttgtcgcggtggttctggccgggaccgcggtagtgtacctgacccatgcctcctcggtacgctatcgtcggctgcgg

SEQ ID NO: 31 - RNA, 1017 nt, synthetic construct
  source 1..1017: mol_type = other RNA
atgggtcggctgaccagcggagttggcaccgccgcactcctggtggtggccgtaggcctgcgggtggtgtgcgccaagtacgccctggccgacccatccctgaagatggccgaccctaaccggttccggggcaagaatctgcccgtgctggatcagctgaccgatcctcctggcgtgaagcgggtgtaccacatccagcccagcctggaggatcccttccagccaccctccatccccatcaccgtttactacgccgtgctggagagagcctgccgcagcgtgctgctgcacgctccatccgaggcgccccagatcgtgcggggcgcaagcgacgaggcccggaagcacacctacaacctgaccatcgcctggtaccggatgggcgacaactgcgccatccctatcaccgtgatggagtacaccgagtgcccctacaacaagagcctgggagtgtgccccatccggacccagcctcggtggtcctactacgacagcttcagcgccgtgtccgaggacaacctgggcttcctgatgcacgcccctgccttcgagaccgccggcacctacctgcggctggtgaagatcaacgactggaccgagatcacccagttcatcctggagcaccgggcaagggccagctgcaagtacgcgctgcctctgcggatccctcccgctgcttgcctgaccagcaaggcctaccagcagggcgtgaccgtggacagcatcggcatgctgcccaggttcatccccgagaaccagcgcaccgtggccctgtacagcctgaagatcgcaggctggcacggacccaagcctccttacacctccaccctgctgccacccgagctgagcgacaccaccaacgccacccagcccgagctggtgcccgaggaccccgaggactccgccctgctggaggatccggccggcaccgtaagctcccagatcccacccaactggcacatccccagcatccaggacgtggccccacatcacgcgcctgccgctccaagcaacccc

SEQ ID NO: 32 - RNA, 1737 nt, synthetic construct
  source 1..1737: mol_type = other RNA
atgggtcggctgaccagcggagttggcaccgccgcactcctggtggtggccgtaggcctgcgggtggtgtgcgccaagtacgccctggccgacccatccctgaagatggccgaccctaaccggttccggggcaagaatctgcccgtgctggatcagggcgtgcccgtgtggaaggaggccaccaccaccctgttctgcgccagcgacgccaaggcctacgacaccgaggtgcacaacgtgtgggccacccacgcctgtgtgcccaccgaccccaacccacaggaggtggtgctggtgaacgtgaccgagaacttcaacatgtggaagaacgacatggtggagcagatgcacgaggacatcatcagcctgtgggaccagagcctgaagccctgcgtgaagctgactcccctgtgcgtgagcctgaagtgcaccgacctgaagaacgacaccaacaccaacagcagcagcggccggatgatcatggagaagggcgagatcaagaactgcagcttcaacatcagcaccagcatccggggcaaggtgcagaaggagtacgccttcttctacaagctggacatcatccccatcgacaacgacaccaccagctacaagctgaccagctgcaacaccagcgtgatcacccaggcctgccccaaggtgagcttcgagcccatccccatccactactgcgccccagccggctttgccatcctgaagtgcaacaacaagaccttcaacggcaccggaccctgcacgaacgtgagcaccgtgcagtgcacccacggaatccggcccgtggtgagcacccagctgctgctgaacggcagcctggccgaggaggaggtggtgatccggagcgtgaacttcaccgacaacgccaagaccatcatcgtgcagctgaacaccagcgtggagatcaactgcacccggcccaacaacaacacccggaagcggatcaggatccaaagaggccccggcagagccttcgtgaccatcggcaagatcggcaacatgcggcaggcccactgcaacatcagccgggccaagtggaacaacaccctgaagcagatcgccagcaagctgcgggagcagttcggcaacaacaagaccatcatcttcaagcagagctcaggcggggatcccgagatcgtgacccacagcttcaactgcggcggcgagttcttctactgcaacagcacccagctgttcaatagcacctggttcaacagcacctggagcaccgagggcagcaacaacaccgagggcagcgacaccatcaccctgccctgccggatcaagcagatcatcaacatgtggcagaaggtgggcaaggccatgtacgcccctcccatcagcggccagatccggtgcagcagcaacatcaccggcctgctgctgacccgggacggtggcaacagcaacaacgagagcgagatctttagacccggaggcggcgacatgcgggacaactggcggagcgagctgtacaagtacaaggtggtgaagatcgagccactgggagtggcccctaccaaggccaagcggcgcgtagtgcagggcggcggaagtggtggcggactgatcattggagctctggccgggagcactctggcggtgctggtgatcggcggcatcgccttctgggtgcgtcggagagcccagatggcccctaagcggctgcggctgccacacatacgggacgacgacgcgccaccatcccaccagcccctgttctac

SEQ ID NO: 33 - RNA, 1569 nt, synthetic construct
  source 1..1569: mol_type = other RNA
atgggtcggctgaccagcggagttggcaccgccgcactcctggtggtggccgtaggcctgcgggtggtgtgcgccaagtacgccctggccgacccatccctgaagatggccgaccctaaccggttccggggcaagaatctgcccgtgctggatcagggcgtgcccgtgtggaaggaggccaccaccaccctgttctgcgccagcgacgccaaggcctacgacaccgaggtgcacaacgtgtgggccacccacgcctgtgtgcccaccgaccccaacccacaggaggtggtgctggtgaacgtgaccgagaacttcaacatgtggaagaacgacatggtggagcagatgcacgaggacatcatcagcctgtgggaccagagcctgaagccctgcgtgaagctgactcccctgtgcgtgagcctgaagtgcaccgacctgaagaacgacaccaacaccaacagcagcagcggccggatgatcatggagaagggcgagatcaagaactgcagcttcaacatcagcaccagcatccggggcaaggtgcagaaggagtacgccttcttctacaagctggacatcatccccatcgacaacgacaccaccagctacaagctgaccagctgcaacaccagcgtgatcacccaggcctgccccaaggtgagcttcgagcccatccccatccactactgcgccccagccggctttgccatcctgaagtgcaacaacaagaccttcaacggcaccggaccctgcacgaacgtgagcaccgtgcagtgcacccacggaatccggcccgtggtgagcacccagctgctgctgaacggcagcctggccgaggaggaggtggtgatccggagcgtgaacttcaccgacaacgccaagaccatcatcgtgcagctgaacaccagcgtggagatcaactgcacccggcccaacaacaacacccggaagcggatcaggatccaaagaggccccggcagagccttcgtgaccatcggcaagatcggcaacatgcggcaggcccactgcaacatcagccgggccaagtggaacaacaccctgaagcagatcgccagcaagctgcgggagcagttcggcaacaacaagaccatcatcttcaagcagagctcaggcggggatcccgagatcgtgacccacagcttcaactgcggcggcgagttcttctactgcaacagcacccagctgttcaatagcacctggttcaacagcacctggagcaccgagggcagcaacaacaccgagggcagcgacaccatcaccctgccctgccggatcaagcagatcatcaacatgtggcagaaggtgggcaaggccatgtacgcccctcccatcagcggccagatccggtgcagcagcaacatcaccggcctgctgctgacccgggacggtggcaacagcaacaacgagagcgagatctttagacccggaggcggcgacatgcgggacaactggcggagcgagctgtacaagtacaaggtggtgaagatcgagccactgggagtggcccctaccaaggccaagcggcgcgtagtgcagcgggagaagcgg

SEQ ID NO: 34 - RNA, 1518 nt, synthetic construct
  source 1..1518: mol_type = other RNA
atggtgttcacccctcagatcctgggcctgatgctgttctggatcagcgccagccggggcaacactcccgtggcctacctgatcgtgggtgtgaccgccagcggcagcttcagcaccatccccatcgtgaacgatccccggacgcgcgtcgaagcagaagccgccgtgagagcgggtaccgccgtggacttcatctggaccggcaatcctcggaccgctcctcggagcctgagcctgggcggacacaccgtgagggcccttagccccacccctccttggcccggaaccgacgacgaggacgacgacctggccgacgtggactacgtgcctccggctcctagacgcgctcctagaaggggaggtggcggtgctggagcaactaggggaaccagccagcctgccgctacaagacccgcacctcctggtgcaccgcggagcagcagtagcggtggcgcacctctgcgggcaggcgttggatcaggctccggaggcggaccagctgtggccgccgtggttcctcgggtggccagcctaccacctgcagccggtggaggcagagcgcaagctcgacgggtgggagaggacgcagctgctgccgaaggccggacaccacccgctaggcaaccacgggcagcccaggagcctcccatcgtgatcagcgacagcccgccaccaagtccccggagacctgccggacctggacccctgagcttcgtgagcagcagcagcgcccaggtgtcaagcggtccaggaggcggaggcttaccccagagcagcgggagggctgcaagaccacgcgctgcagtggctccgcgggtgagatctccgccacgtgccgctgcagcccctgtggtgagcgcatccgctgacgctgccggtcctgcacctcctgccgtgcccgttgacgcccatagagctccccggagccggatgacccaggcccagaccgacacccaagctcaaagcctgggtcgtgccggtgcaacagacgccaggggcagtggtggaccaggtgcagagggcgggcctggagttccacggggaaccaacacccctggtgccgctccacacgctgccgaaggggctgctgctggcggaggcagtggaggtggctctccagcccctggactgacccggtacctgcccatcgccggcgtgagcagcgtggtggccctggctccctacgtgaacaagaccgtgaccggcgactgcttacccgtgctggacatggagaccggccacatcggcgcctacgtggtgctggtggaccagaccggcaacgtggccgacctcctgagagccgccgcaccagcttggagccggagaaccctgctgcctgagcacgcccggaactgcgtgcggcctcccgactaccccacaccgcctgccagcgagtggaacagcctgtggatgacgcccgtgggcaacatgctgttcgaccagggcaccctggtgggagccctggacttccacggcctgaggagccggcacccttggagccgggagcaaggcgcaccc

SEQ ID NO: 35 - RNA, 2118 nt, synthetic construct
  source 1..2118: mol_type = other RNA
atggtgttcacccctcagatcctgggcctgatgctgttctggatcagcgccagccggggccgggtgctgtacggtggcctgggcgactcaagacctggcctctggggcgcacctgaggcagaggaagcgcgggctcgttttgaggcatctggtgcccctgcccctgtgtgggccccagagctgggagacgccgcccagcagtacgccctgatcacccggctgctgtacacacccgacgccgaggccatgggctggctgcagaaccctcgggtggctccaggggacgtggccctggaccaggcctgcttcagaataagcggggccgctcggaacagcagcagcttcatcagcggcagcgttgctcgagccgtgcctcacctgggctacgcaatggccgccggcagattcggctggggcctggctcacgtcgctgcggcagtggctatgagccggcggtacgacagggcccagaagggcttcctgttgacaagcctgcgccgcgcatacgctccactgttagcccgcgagaacgccgcactgacgggagcccggactcctgacgacggcggagacgccaacagacacgacggggacgacgcaagaggtaaaccgcccctgcccagtgctgcggcctctcccgctgacgagagagccgtgcctgcaggctacggcgcagctggggtgctggcagcactgggcaggctgtcagccgcccctgctagcgctccagctggcgctagggctgaagcaggcagggtggccgtggagtgcttagccgcctgtcggggcatcctggaggccctggccgaaggctttgacggggacctggctgcggtgcctggcttggccggtgccagacctgcagcacctccccgacctggtccagcaggcgcagcagctcctcctcacgctgacgccccacgcctgagagcctggctgcgggagctgcggttcgtgcgggacgccctggtgctgatgcggctgagaggcgacctgcgtgttgctggcggttccgaggcagctgtggccgcagtgagagccgttagcctggtggccggcgcattaggaccagccctgccacgtagcccgagactgctgagcagcgacctgctgttccagaatcagtctttgggtggagggtccggtggaggacctcctccaggtggagcacctgccgccttcggccctttaagggcgtctggccctctgcgtagagcagccgcctggatgcggcaggtgcccgatcccgaggacgtgcgggtggtgatcctgtacagtcccctgcccggcgaagatctcgcagccggaagagccggaggcggaccaccacctgagtggagcgccgaacgaggtgggctgagctgtcttctcgccgcgctgggcaacagactctgtggacccgctacagccgcctgggctggcaattggacaggtgccccggacgtatccgccctgggcgcacaaggcgtgctgctgctgagcacccgggatctggcattcgccggcgccgtggagtttctgggactgctggcaggtgcctgtgaccggcggctgatcgtggtcaacgcagtcagagccgctgactggcctgcggacggacctgtggtgagccggcagcacgcctacctggcctgcgaggtgctgccagccgtgcagtgtgcagttcggtggcctgccgccagagacctgcggcggaccgtgttagccagcggccgggttttcggtcccggagtgttcgctcgggtggaagcagcccacgcacggctgtaccccgacgccccacctctgcggctgtgcagaggagcaaacgtgcggtaccgggtgcggacacggttcggcccagacaccctggtgcccatgtcaccccgggagtaccggagagccgtgttacctgcccttgacggaagggcagccgcttctggggccggtgacgctatggctcctggcgctcccgacttctgcgaggacgaagcccacagccaccgagcctgcgccaggtggggtctcggtgccccactgagacccgtgtacgtggctctgggacgggacgctgtacggggtgggcccgctgaacttagagggccacggcgggagttctgtgcacgggccctgctggag

SEQ ID NO: 36 - protein, 901 aa, unidentified
  source 1..901: note = Herpes simplex virus
MRGGGLVCALVVGALVAAVASAAPAAPRASGGVAATVAANGGPASQPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 37 - protein, 901 aa, synthetic construct
MRGGGLVCALVVGALVAAVASAAPAAPRASGGVAATVAANGGPASQPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRPVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 38 - protein, 480 aa, synthetic construct
MALGRVGLAVGLWGLLWVGVVVVLANASPGRTITVGPRGNASNAAPSASPRNASAPRTTPTPPQPRKATKSKASTAKPAPPPKTGPPKTSSEPVRCNRHDPLARYGSRVQIRCRFPNSTRTESRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSASRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHHGSHQPPPRDPTERQVIRAVEGAGIGVAVLVAVVLAGTAVVYLTHASSVRYRRLR

SEQ ID NO: 39 - protein, 393 aa, unidentified
  source 1..393: note = Herpes simplex virus
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 40 - protein, 548 aa, unidentified
  source 1..548: note = Herpes simplex virus
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 41 - protein, 417 aa, synthetic construct
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLR

SEQ ID NO: 42 - protein, 838 aa, unidentified
  source 1..838: note = Herpes simplex virus
MGPGLWVVMGVLVGVAGGHDTYWTEQIDPWFLHGLGLARTYWRDTNTGRLWLPNTPDASDPQRGRLAPPGELNLTTASVPMLRWYAERFCFVLVTTAEFPRDPGQLLYIPKTYLLGRPRNASLPELPEAGPTSRPPAEVTQLKGLSHNPGASALLRSRAWVTFAAAPDREGLTFPRGDDGATERHPDGRRNAPPPGPPAGTPRHPTTNLSIAHLHNASVTWLAARGLLRTPGRYVYLSPSASTWPVGVWTTGGLAFGCDAALVRARYGKGFMGLVISMRDSPPAEIIVVPADKTLARVGNPTDENAPAVLPGPPAGPRYRVFVLGAPTPADNGSALDALRRVAGYPEESTNYAQYMSRAYAEFLGEDPGSGTDARPSLFWRLAGLLASSGFAFVNAAHAHDAIRLSDLLGFLAHSRVLAGLAARGAAGCAADSVFLNVSVLDPAARLRLEARLGHLVAAILEREQSLAAHALGYQLAFVLDSPAAYGAVAPSAARLIDALYAEFLGGRALTAPMVRRALFYATAVLRAPFLAGAPSAEQRERARRGLLITTALCTSDVAAATHADLRAALARTDHQKNLFWLPDHFSPCAASLRFDLAEGGFILDALAMATRSDIPADVMAQQTRGVASALTRWAHYNALIRAFVPEATHQCSGPSHNAEPRILVPITHNASYVVTHTPLPRGIGYKLTGVDVRRPLFITYLTATCEGHAREIEPKRLVRTENRRDLGLVGAVFLRYTPAGEVMSVLLVDTDATQQQLAQGPVAGTPNVFSSDVPSVALLLFPNGTVIHLLAFDTLPIATIAPGFLAASALGVVMITAALAGILRVVRTCVPFLWRRE

SEQ ID NO: 43 - protein, 224 aa, unidentified
  source 1..224: note = Herpes simplex virus
MGFVCLFGLVVMGAWGAWGGSQATEYVLRSVIAKEVGDILRVPCMRTPADDVSWRYEAPSVIDYARIDGIFLRYHCPGLDTFLWDRHAQRAYLVNPFLFAAGFLEDLSHSVFPADTQETTTRRALYKEIRDALGSRKQAVSHAPVRAGCVNFDYSRTRRCVGRRDLRPANTTSTWEPPVSSDDEASSQSKPLATQPPVLALSNAPPRRVSPTRGRRRHTRLRRN

SEQ ID NO: 44 - protein, 209 aa, synthetic construct
MAWGGSQATEYVLRSVIAKEVGDILRVPCMRTPADDVSWRYEAPSVIDYARIDGIFLRYHCPGLDTFLWDRHAQRAYLVNPFLFAAGFLEDLSHSVFPADTQETTTRRALYKEIRDALGSRKQAVSHAPVRAGCVNFDYSRTRRCVGRRDLRPANTTSTWEPPVSSDDEASSQSKPLATQPPVLALSNAPPRRVSPTRGRRRHTRLRRN

SEQ ID NO: 45 - protein, 1076 aa, synthetic construct
MGFVCLFGLVVMGAWGAWGGSQATEYVLRSVIAKEVGDILRVPCMRTPADDVSWRYEAPSVIDYARIDGIFLRYHCPGLDTFLWDRHAQRAYLVNPFLFAAGFLEDLSHSVFPADTQETTTRRALYKEIRDALGSRKQAVSHAPVRAGCVNFDYSRTRRCVGRRDLRPANTTSTWEPPVSSDDEASSQSKPLATQPPVLALSNAPPRRVSPTRGRRRHTRRRRRGSGGSGSGGSSGGGSGSGGSGGSGSGGRRRRRHDTYWTEQIDPWFLHGLGLARTYWRDTNTGRLWLPNTPDASDPQRGRLAPPGELNLTTASVPMLRWYAERFCFVLVTTAEFPRDPGQLLYIPKTYLLGRPRNASLPELPEAGPTSRPPAEVTQLKGLSHNPGASALLRSRAWVTFAAAPDREGLTFPRGDDGATERHPDGRRNAPPPGPPAGTPRHPTTNLSIAHLHNASVTWLAARGLLRTPGRYVYLSPSASTWPVGVWTTGGLAFGCDAALVRARYGKGFMGLVISMRDSPPAEIIVVPADKTLARVGNPTDENAPAVLPGPPAGPRYRVFVLGAPTPADNGSALDALRRVAGYPEESTNYAQYMSRAYAEFLGEDPGSGTDARPSLFWRLAGLLASSGFAFVNAAHAHDAIRLSDLLGFLAHSRVLAGLAARGAAGCAADSVFLNVSVLDPAARLRLEARLGHLVAAILEREQSLAAHALGYQLAFVLDSPAAYGAVAPSAARLIDALYAEFLGGRALTAPMVRRALFYATAVLRAPFLAGAPSAEQRERARRGLLITTALCTSDVAAATHADLRAALARTDHQKNLFWLPDHFSPCAASLRFDLAEGGFILDALAMATRSDIPADVMAQQTRGVASALTRWAHYNALIRAFVPEATHQCSGPSHNAEPRILVPITHNASYVVTHTPLPRGIGYKLTGVDVRRPLFITYLTATCEGHAREIEPKRLVRTENRRDLGLVGAVFLRYTPAGEVMSVLLVDTDATQQQLAQGPVAGTPNVFSSDVPSVALLLFPNGTVIHLLAFDTLPIATIAPGFLAASALGVVMITAALAGILRVVRTCVPFLWRRE

SEQ ID NO: 46 - protein, 753 aa, synthetic construct
MEPRPGTSSRADPGPERPPRQTPGTQPAAPHAWGMLNDMQWLASSDSEEETEVGISDDDLHRDSTSEAGSTDTEMFEAGLMDAATPPARPPAERQGSPTPADAQGSCGGGPVGEEEAEAGGGGDVNTPVAYLIVGVTASGSFSTIPIVNDPRTRVEAEAAVRAGTAVDFIWTGNPRTAPRSLSLGGHTVRALSPTPPWPGTDDEDDDLADVDYVPPAPRRAPRRGGGGAGATRGTSQPAATRPAPPGAPRSSSSGGAPLRAGVGSGSGGGPAVAAVVPRVASLPPAAGGGRAQARRVGEDAAAAEGRTPPARQPRAAQEPPIVISDSPPPSPRRPAGPGPLSFVSSSSAQVSSGPGGGGLPQSSGRAARPRAAVAPRVRSPPRAAAAPVVSASADAAGPAPPAVPVDAHRAPRSRMTQAQTDTQAQSLGRAGATDARGSGGPGAEGGSGPAASSSASSSAAPRSPLAPQGVGAKRAAPRRAPDSDSGDRGHGPLAPASAGAAPPSASPSSQAAVAAASSSSASSSSASSSSASSSSASSSSASSSSASSSSASSSAGGAGGSVASASGAGERRETSLGPRAAAPRGPRKCARKTRHAEGGPEPGARDPAPGLTRYLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGALDFHGLRSRHPWSREQGAPAPAGDAPAGHGE

SEQ ID NO: 47 - protein, 487 aa, synthetic construct
MNTPVAYLIVGVTASGSFSTIPIVNDPRTRVEAEAAVRAGTAVDFIWTGNPRTAPRSLSLGGHTVRALSPTPPWPGTDDEDDDLADVDYVPPAPRRAPRRGGGGAGATRGTSQPAATRPAPPGAPRSSSSGGAPLRAGVGSGSGGGPAVAAVVPRVASLPPAAGGGRAQARRVGEDAAAAEGRTPPARQPRAAQEPPIVISDSPPPSPRRPAGPGPLSFVSSSSAQVSSGPGGGGLPQSSGRAARPRAAVAPRVRSPPRAAAAPVVSASADAAGPAPPAVPVDAHRAPRSRMTQAQTDTQAQSLGRAGATDARGSGGPGAEGGPGVPRGTNTPGAAPHAAEGAAAGGGSGGGSPAPGLTRYLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGALDFHGLRSRHPWSREQGAP

SEQ ID NO: 48 - protein, 1153 aa, synthetic construct
MSAEQQGRGAEVAMADEDGGRLRAAAETTGGPGSPDPADGPPPTPNPDRRPAARPGFGWHGGPEENEDEADASGEAVDEPAADGVVSPRQLALLASMVDEAVRTIPSPPPERDGAQEEAARSPSPPRTPSMRADYGEENAGRWVRGPETTSAVRGAYPDPMASLSPRPPAPAARAPASAADHAAGGTLGADDEEAGVPARAPGAAPRPSPPRAEPAPARTPAATAGRLERRRARAAVAGRDATGRFTAGRPRRVELDADAASGAFYARYRDGYVSGEPWPGAGPPPPGRVLYGGLGDSRPGLWGAPEAEEARARFEASGAPAPVWAPELGDAAQQYALITRLLYTPDAEAMGWLQNPRVAPGDVALDQACFRISGAARNSSSFISGSVARAVPHLGYAMAAGRFGWGLAHVAAAVAMSRRYDRAQKGFLLTSLRRAYAPLLARENAALTGARTPDDGGDANRHDGDDARGKPPLPSASPADERAVPAGYGAAGVLAALGRLSAAPASAPAGARAEAGRVAVECLAACRGILEALAEGFDGDLAAVPGLAGARPAAPPRPGPAGAAAPPHADAPRLRAWLRELRFVRDALVLMRLRGDLRVAGGSEAAVAAVRAVSLVAGALGPALPRSPRLLSSDLLFQNQSLRPLLADTVAAADSLAAPASAPREAADAPRPAAARPAALTRRPAEGPDPQGGWRRQPPGPSHTPAPSAAALEAYCAPRAVAELTDHPLFPAPWRPALMFDPRALASLAARCAAPPPGGAPAAFGPLRASGPLRRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAAAWPAAAPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGAGDAMAPGAPDFCEDEAHSHRACARWGLGAPLRPVYVALGRDAVRGGPAELRGPRREFCARALLEPDGDAPPLVLRDDADAGPPPQIRWASAAGRAGTVLAAAGGGVEVVGTAAGLATPPRREPVDMDAELEDDDDGLFGE

SEQ ID NO: 49 - protein, 687 aa, synthetic construct
MRVLYGGLGDSRPGLWGAPEAEEARARFEASGAPAPVWAPELGDAAQQYALITRLLYTPDAEAMGWLQNPRVAPGDVALDQACFRISGAARNSSSFISGSVARAVPHLGYAMAAGRFGWGLAHVAAAVAMSRRYDRAQKGFLLTSLRRAYAPLLARENAALTGARTPDDGGDANRHDGDDARGKPPLPSAAASPADERAVPAGYGAAGVLAALGRLSAAPASAPAGARAEAGRVAVECLAACRGILEALAEGFDGDLAAVPGLAGARPAAPPRPGPAGAAAPPHADAPRLRAWLRELRFVRDALVLMRLRGDLRVAGGSEAAVAAVRAVSLVAGALGPALPRSPRLLSSDLLFQNQSLGGGSGGGPPPGGAPAAFGPLRASGPLRRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGAGDAMAPGAPDFCEDEAHSHRACARWGLGAPLRPVYVALGRDAVRGGPAELRGPRREFCARALLE

SEQ ID NO: 50 - protein, 901 aa, synthetic construct
MRGGGLVCALVVGALVAAVASAAPAAPRASGGVAATVAANGGPASQPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRPPVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 51 - protein, 901 aa, synthetic construct
MRGGGLVCALVVGALVAAVASAAPAAPRASGGVAATVAANGGPASQPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDPPGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 52 - protein, 901 aa, synthetic construct
MRGGGLVCALVVGALVAAVASAAPAAPRASGGVAATVAANGGPASQPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRPPNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 53 - protein, 901 aa, synthetic construct
MRGGGLVCALVVGALVAAVASAAPAAPRASGGVAATVAANGGPASQPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNPPQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 54 - protein, 798 aa, synthetic construct
MRGGGLVCALVVGALVAAVASAAPAAPRASGGVAATVAANGGPASQPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRN

SEQ ID NO: 55 - protein, 798 aa, synthetic construct
MRGGGLVCALVVGALVAAVASAAPAAPRASGGVAATVAANGGPASQPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDPPGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRN

SEQ ID NO: 56 - protein, 798 aa, synthetic construct
MRGGGLVCALVVGALVAAVASAAPAAPRASGGVAATVAANGGPASQPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNPPQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRN

SEQ ID NO: 57 - protein, 798 aa, synthetic construct
MRGGGLVCALVVGALVAAVASAAPAAPRASGGVAATVAANGGPASQPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNPPQRPPNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRN

SEQ ID NO: 58 - protein, 372 aa, unidentified
  source 1..372: note = Herpes simplex virus
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTSPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 59 - protein, 254 aa, synthetic construct
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGEIAPPNSTRSASESRHR

SEQ ID NO: 60 - protein, 434 aa, synthetic construct
MARGAGLVFFVGVWVVSCLAAAPRTSWKRVTSGEDVVLLPAPAERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWA

SEQ ID NO: 61 - protein, 283 aa, synthetic construct
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGEIAPPNSTRSASESRHRLTVAQVIQIAIPASIIAFVFLGSCICFIH

SEQ ID NO: 62 - protein, 469 aa, synthetic construct
MALGRVGLAVGLWGLLWVGVVVVLANASPGRTITVGPRGNASNAAPSASPRNASAPRTTPTPPQPRKATKSKASTAKPAPPPKTGPPKTSSEPVRCNRHDPLARYGSRVQIRCRFPNSTRTESRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHHGSHQPPPRDPTERQVIRAVEGAGIGVAVLVAVVLAGTAVVYLT

SEQ ID NO: 63 - protein, 469 aa, synthetic construct
MALGRVGLAVGLWGLLWVGVVVVLANASPGRTITVGPRGNASNAAPSASPRNASAPRTTPTPPQPRKATKSKASTAKPAPPPKTGPPKTSSEPVRCNRHDPLARYGSRVQIRCRFPNSTRTESRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSASRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHHGSHQPPPRDPTERQVIRAVEGAGIGVAVLVAVVLAGTAVVYLT

SEQ ID NO: 64 - protein, 363 aa, synthetic construct
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWV

SEQ ID NO: 65 - protein, 480 aa, unidentified
  source 1..480: note = Herpes simplex virus
MALGRVGLAVGLWGLLWVGVVVVLANASPGRTITVGPRGNASNAAPSASPRNASAPRTTPTPPQPRKATKSKASTAKPAPPPKTGPPKTSSEPVRCNRHDPLARYGSRVQIRCRFPNSTRTESRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHHGSHQPPPRDPTERQVIRAVEGAGIGVAVLVAVVLAGTAVVYLTHASSVRYRRLR

SEQ ID NO: 66 - protein, 339 aa, synthetic construct
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNP

SEQ ID NO: 67 - protein, 579 aa, synthetic construct
MGRLTSGVGTAALLVVAVGLRVVCAKYALADPSLKMADPNRFRGKNLPVLDQGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQGGGSGGGLIIGALAGSTLAVLVIGGIAFWVRRRAQMAPKRLRLPHIRDDDAPPSHQPLFY

SEQ ID NO: 68 - protein, 523 aa, synthetic construct
MGRLTSGVGTAALLVVAVGLRVVCAKYALADPSLKMADPNRFRGKNLPVLDQGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 69 - protein, 506 aa, synthetic construct
MVFTPQILGLMLFWISASRGNTPVAYLIVGVTASGSFSTIPIVNDPRTRVEAEAAVRAGTAVDFIWTGNPRTAPRSLSLGGHTVRALSPTPPWPGTDDEDDDLADVDYVPPAPRRAPRRGGGGAGATRGTSQPAATRPAPPGAPRSSSSGGAPLRAGVGSGSGGGPAVAAVVPRVASLPPAAGGGRAQARRVGEDAAAAEGRTPPARQPRAAQEPPIVISDSPPPSPRRPAGPGPLSFVSSSSAQVSSGPGGGGLPQSSGRAARPRAAVAPRVRSPPRAAAAPVVSASADAAGPAPPAVPVDAHRAPRSRMTQAQTDTQAQSLGRAGATDARGSGGPGAEGGPGVPRGTNTPGAAPHAAEGAAAGGGSGGGSPAPGLTRYLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGALDFHGLRSRHPWSREQGAP

SEQ ID NO: 70 - protein, 706 aa, synthetic construct
MVFTPQILGLMLFWISASRGRVLYGGLGDSRPGLWGAPEAEEARARFEASGAPAPVWAPELGDAAQQYALITRLLYTPDAEAMGWLQNPRVAPGDVALDQACFRISGAARNSSSFISGSVARAVPHLGYAMAAGRFGWGLAHVAAAVAMSRRYDRAQKGFLLTSLRRAYAPLLARENAALTGARTPDDGGDANRHDGDDARGKPPLPSAAASPADERAVPAGYGAAGVLAALGRLSAAPASAPAGARAEAGRVAVECLAACRGILEALAEGFDGDLAAVPGLAGARPAAPPRPGPAGAAAPPHADAPRLRAWLRELRFVRDALVLMRLRGDLRVAGGSEAAVAAVRAVSLVAGALGPALPRSPRLLSSDLLFQNQSLGGGSGGGPPPGGAPAAFGPLRASGPLRRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGAGDAMAPGAPDFCEDEAHSHRACARWGLGAPLRPVYVALGRDAVRGGPAELRGPRREFCARALLE

SEQ ID NO: 71 - protein, 20 aa, Homo sapiens
METPAQLLFLLLLWLPDTTG

SEQ ID NO: 72 - protein, 18 aa, Homo sapiens
MDWTWILFLVAAATRVHS

SEQ ID NO: 73 - protein, 24 aa, Japanese encephalitis virus
MLGSNSGQRVVFTILLLLVAPAYS

SEQ ID NO: 74 - protein, 17 aa, unidentified
  source 1..17: note = vesicular stomatitis virus
MKCLLYLAFLFIGVNCA

SEQ ID NO: 75 - protein, 15 aa, Japanese encephalitis virus
MWLVSLAIVTACAGA

SEQ ID NO: 76 - protein, 15 aa, synthetic construct
GGSGGSGGSGGSGGG

SEQ ID NO: 77 - protein, 8 aa, synthetic construct
GGGSGGGS

SEQ ID NO: 78 - protein, 7 aa, synthetic construct
GGGSGGG

SEQ ID NO: 79 - protein, 4 aa, synthetic construct
GGGS

SEQ ID NO: 80 - protein, 20 aa, synthetic construct
  SITE 5..20: note = may be absent
GGGSGGGSGGGSGGGSGGGS

SEQ ID NO: 81 - protein, 4 aa, synthetic construct
GSGG

SEQ ID NO: 82 - protein, 6 aa, synthetic construct
GGGSGG

SEQ ID NO: 83 - RNA, 47 nt, synthetic construct
  source 1..47: mol_type = other RNA
gggaaataagagagaaaagaagagtaagaagaaatataagagccacc

SEQ ID NO: 84 - RNA, 57 nt, synthetic construct
  source 1..57: mol_type = other RNA
gggaaataagagagaaaagaagagtaagaagaaatataagaccccggcgccgccacc

SEQ ID NO: 85 - RNA, 77 nt, synthetic construct
  source 1..77: mol_type = other RNA
gaggaaatcgcaaaatttgctcttcgcgttagatttcttttagttttctcgcaactagcaagctttttgttctcgcc

SEQ ID NO: 86 - RNA, 119 nt, synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 87 - RNA, 119 nt, synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 88 - RNA, 129 nt, synthetic construct
  source 1..129: mol_type = other RNA
taaagctccccgggggcctcggtggcctagcttcttgccccttgggcctccccccagcccctcctccccttcctgcaggagattgagtgtagtgactagtggtctttgaataaagtctgagtgggcggc

SEQ ID NO: 89 - RNA, 123 nt, synthetic construct
  source 1..123: mol_type = other RNA
taaagctccccgggggcctcggtggcctagcttcttgccccttgggcctccccccagcccctcctccccttcctgcaggattgagactacgggtggtctttgaataaagtctgagtgggcggc

SEQ ID NO: 90 - RNA, 121 nt, synthetic construct
  source 1..121: mol_type = other RNA
taaagctccccgggggcctcggtggcctagcttcttgccccttgggcctccccccagcccctcctccccttcctgcagcatagacactacgtggtctttgaataaagtctgagtgggcggc

SEQ ID NO: 91 - RNA, 125 nt, synthetic construct
  source 1..125: mol_type = other RNA
taaagctccccgggggcctcggtggcctagcttcttgccccttgggcctccccccagcccctcctccccttcctgcaggagattgagtgtagtggtggtctttgaataaagtctgagtgggcggc

SEQ ID NO: 92 - RNA, 127 nt, synthetic construct
  source 1..127: mol_type = other RNA
taaagctccccgggggcctcggtggcctagcttcttgccccttgggcctccccccagcccctcctccccttcctgcaggagattgagtgtagtgacgtggtctttgaataaagtctgagtgggcggc

SEQ ID NO: 93 - RNA, 108 nt, synthetic construct
  source 1..108: mol_type = other RNA
taaagctccccgggggcctcggtggcctagcttcttgccccttgggcctccccccagcccctcctccccttcctgcagtggtctttgaataaagtctgagtgggcggc

SEQ ID NO: 94 - RNA, 78 nt, synthetic construct
  source 1..78: mol_type = other RNA
taaagctccccgggggcctcggtggcctagcttcttgccccttgggcctccccccagcccctcctccccttcctgcag

SEQ ID NO: 95 - RNA, 30 nt, synthetic construct
  source 1..30: mol_type = other RNA
tggtctttgaataaagtctgagtgggcggc

SEQ ID NO: 96 - RNA, 10 nt, synthetic construct
  source 1..10: mol_type = other RNA
ccrrccatgg

SEQ ID NO: 97 - RNA, 11 nt, synthetic construct
  source 1..11: mol_type = other RNA
gggatcctacc

SEQ ID NO: 98 - RNA, 21 nt, synthetic construct
  source 1..21: mol_type = other RNA
gagattgagtgtagtgactag

SEQ ID NO: 99 - RNA, 18 nt, synthetic construct
  source 1..18: mol_type = other RNA
gagattgagtgtagtgac

SEQ ID NO: 100 - RNA, 16 nt, synthetic construct
  source 1..16: mol_type = other RNA
gagattgagtgtagtg

SEQ ID NO: 101 - RNA, 15 nt, synthetic construct
  source 1..15: mol_type = other RNA
gattgagactacggg

SEQ ID NO: 102 - RNA, 13 nt, synthetic construct
  source 1..13: mol_type = other RNA
catagacactacg

SEQ ID NO: 103 - protein, 8 aa, unidentified
  source 1..8: note = Herpes simplex virus
SSIEFARL

SEQ ID NO: 104 - RNA, 2600 nt, synthetic construct
  source 1..2600: mol_type = other RNA
gaggaaatcgcaaaatttgctcttcgcgttagatttcttttagttttctcgcaactagcaagctttttgttctcgccatgcggggtggcggactggtgtgcgccctggtggtcggtgcacttgtggccgctgtggctagcgctgcaccagccgcacccagagccagcggaggtgtggccgcgaccgttgcagccaacggaggcccggcttctcagcctcctcctgtgcccagccccgctaccaccaaggcccgtaagcggaagaccaagaagcctcccaagcggcccgaggctacaccaccacccgacgccaacgcaactgttgctgctggccacgccaccctgagggcccacctgcgggagatcaaggtggagaacgccgacgcccagttctacgtgtgtccacctccaaccggcgccaccgtggtacagttcgagcaacctcgccggtgccccactcgacccgagggccagaactacaccgagggcatcgccgtggtgttcaaggagaacatcgccccatacaagttcaaggccaccatgtactacaaggacgtgaccgtgagccaggtgtggttcggccaccggtacagccagttcatgggcatcttcgaggatcgggccccagtgcccttcgaggaggtgatcgacaagatcaacgccaagggcgtgtgccggagcaccgccaagtacgtgcggaacaacatggagaccaccgccttccaccgggacgaccacgagaccgacatggagctgaagcccgccaaggtggccacacggacaagccggggctggcacaccaccgacctgaagtacaacccaagccgcgtggaggcgttccaccggtacggcaccaccgtgaactgcatcgtggaggaggtggacgcccggagcgtgtacccctacgacgaattcgtgctggccaccggcgacttcgtgtacatgagccccttctacggctaccgggagggcagccacaccgagcacaccagctacgccgccgaccggttcaagcaggtggacggcttctacgcccgggacctgaccactaaagcccgggccacctcaccaaccacccggaacctgctgaccaccccaaagttcaccgtggcctgggactgggtgcccaagcgacccgccgtgtgcaccatgaccaagtggcaggaagtggacgagatgctgcgggccgaatacggcggcagcttccggttcagcagcgacgccatcagcaccaccttcaccaccaacctgacccagtacagcctgagccgggtggatctgggcgactgcatcggcagagacgctcgcgaggccatcgaccggatgttcgcccggaagtacaacgccacccacatcaaggtgggccagccccagtactacctcgccaccggcggcttcctgatcgcctaccagcccttgctgagcaacaccctggccgagctgtacgtgcgggagtacatgcgggagcaggaccggaagccccggaacgccacacccgcccctctgagagaagccccttccgccaacgccagcgtggagcggatcaagaccaccagcagcatcgagttcgcccggctgcagttcacctacaaccacatccagcggcacgtgaacgacatgctgggtcggatcgccgtggcttggtgcgagctgcagaaccacgagctgaccctgtggaacgaggcccggaagctgaaccccaacgccatcgccagcgctactgtgggccggagggtaagcgctcggatgctgggcgacgtgatggccgtgagcacgtgtgtgcccgtggccccagacaacgtgatcgtgcagaacagcatgcgggtgagtagccggcctggaacctgctacagccggcccctggtgtctttccggtacgaggaccagggtcccctgatcgagggccagctgggcgagaacaacgagctgcggctgactcgcgacgctctggagccctgcaccgtgggccacagacggtacttcatcttcggcggcggctacgtgtacttcgaggagtacgcctacagccaccagctgagccgggccgacgtgaccaccgtgagcaccttcatcgacctgaacatcaccatgctggaggaccacgagttcgtgcccctggaggtgtacacccggcacgagatcaaggacagcggcctgctggattacaccgaggtgcagcggcggaaccagctgcacgacctgcggttcgccgacatcgacaccgtgatacgggccgacgcaaacgccgccatgttcgccggcctgtgcgccttcttcgagggcatgggcgacctgggcagagccgtgggcaaggtggtgatgggcgtggtgggcggagtggtaagcgccgtgagcggcgtgagcagcttcatgagcaaccccttcggtgccctggcagtgggcctgctggtgcttgctggcctggtcgctgccttcttcgccttccggtacgtgctacagctgcagcggaactaaagctccccgggggcctcggtggcctagcttcttgccccttgggcctccccccagcccctcctccccttcctgcaggagattgagtgtagtgactagtggtctttgaataaagtctgagtgggcggc

SEQ ID NO: 105 - RNA, 1607 nt, synthetic construct
  source 1..1607: mol_type = other RNA
gaggaaatcgcaaaatttgctcttcgcgttagatttcttttagttttctcgcaactagcaagctttttgttctcgccatggcacttggacgcgtcggtctggctgtcggcctgtggggcctgctgtgggtgggcgtggtggtggtgctggctaacgccagccccggacgtaccatcaccgtggggcccagaggcaacgccagtaacgccgcgccatcagccagcccgagaaacgctagtgctccccggaccacgccaactccaccacagccccggaaggccaccaagagcaaggccagcaccgccaagcccgctccacctcccaagaccggcccacccaagaccagcagcgagcccgtgcggtgcaaccggcacgatcctctggcccggtacggctcacgggtgcagatccggtgccggttccccaacagcaccaggaccgagagccggctgcagatctggcggtacgccaccgccacagacgccgagatcggcaccgccccaagcctggaggaggtgatggtgaacgtgtctgctccacctggcggccagctggtgtacgacagcgcacccaaccggaccgatccccacgtgatctgggcagaaggcgctggtcctggcgctagccctcgactgtacagcgtggtcggcccactgggcaggcagcggctgatcatcgaggagctgaccctggagacccagggcatgtactactgggtgtggggccggacagatcggcctagcgcctacgggacctgggtgcgggttcgcgtgttccggccacctagcctgaccatccatccccacgccgtgctggagggccagcccttcaaagccacctgtaccgccgccacctactacccaggcaaccgggccgagttcgtgtggttcgaggacggacggcgcgtgttcgaccccgcccagatccacacccagacccaggagaaccccgacggcttcagcaccgtgagcacggtgaccagcgctgccgttggaggccaaggccctcccagaaccttcacctgccagctgacctggcaccgggacagcgtgagcgcttctcgccggaacgccagcggaactgccagcgtgctgccacggcccaccatcaccatggagttcaccggcgaccacgccgtgtgcacagccggctgcgtacccgagggcgtgaccttcgcctggttcctgggcgacgacagcagccccgccgagaaggtggccgtggccagccagaccagctgtggaagacccggaaccgccaccatccggagcaccctgcccgtgagctacgagcagaccgagtacatctgtcggctggccggctaccctgacggcatccccgtgttggagcaccacggcagccaccagcctcctcctcgggatcccaccgagcggcaggtgatcagggccgtggagggtgcaggcatcggcgtggccgtgctggtggccgtagtgctagccggcaccgccgtggtatacctgacctaaagctccccgggggcctcggtggcctagcttcttgccccttgggcctccccccagcccctcctccccttcctgcaggattgagactacgggtggtctttgaataaagtctgagtgggcggc

SEQ ID NO: 106 - RNA, 1377 nt, synthetic construct
  source 1..1377: mol_type = other RNA
gaggaaatcgcaaaatttgctcttcgcgttagatttcttttagttttctcgcaactagcaagctttttgttctcgccatgggtcggctgaccagcggagttggcaccgccgcactcctggtggtggccgtaggcctgcgggtggtgtgcgccaagtacgccctggccgacccatccctgaagatggccgaccctaaccggttccggggcaagaatctgcccgtgctggatcagctgaccgatcctcctggcgtgaagcgggtgtaccacatccagcccagcctggaggatcccttccagccaccctccatccccatcaccgtttactacgccgtgctggagagagcctgccgcagcgtgctgctgcacgctccatccgaggcgccccagatcgtgcggggcgcaagcgacgaggcccggaagcacacctacaacctgaccatcgcctggtaccggatgggcgacaactgcgccatccctatcaccgtgatggagtacaccgagtgcccctacaacaagagcctgggagtgtgccccatccggacccagcctcggtggtcctactacgacagcttcagcgccgtgtccgaggacaacctgggcttcctgatgcacgcccctgccttcgagaccgccggcacctacctgcggctggtgaagatcaacgactggaccgagatcacccagttcatcctggagcaccgggcaagggccagctgcaagtacgcgctgcctctgcggatccctcccgctgcttgcctgaccagcaaggcctaccagcagggcgtgaccgtggacagcatcggcatgctgcccaggttcatccccgagaaccagcgcaccgtggccctgtacagcctgaagatcgcaggctggcacggacccaagcctccttacacctccaccctgctgccacccgagctgagcgacaccaccaacgccacccagcccgagctggtgcccgaggaccccgaggactccgccctgctggaggatccggccggcaccgtaagctcccagatcccacccaactggcacatccccagcatccaggacgtggccccacatcacgcgcctgccgctccaagcaaccccggcctgatcattggagctctggccgggagcactctggcggtgctggtgatcggcggcatcgccttctgggtgcgtcggagagcccagatggcccctaagcggctgcggctgccacacatacgggacgacgacgcgccaccatcccaccagcccctgttctactaaagctccccgggggcctcggtggcctagcttcttgccccttgggcctccccccagcccctcctccccttcctgcagcatagacactacgtggtctttgaataaagtctgagtgggcggc

SEQ ID NO: 107 - RNA, 1663 nt, synthetic construct
  source 1..1663: mol_type = other RNA
gaggaaatcgcaaaatttgctcttcgcgttagatttcttttagttttctcgcaactagcaagctttttgttctcgccatgaacactcccgtggcctacctgatcgtgggtgtgaccgccagcggcagcttcagcaccatccccatcgtgaacgatccccggacgcgcgtcgaagcagaagccgccgtgagagcgggtaccgccgtggacttcatctggaccggcaatcctcggaccgctcctcggagcctgagcctgggcggacacaccgtgagggcccttagccccacccctccttggcccggaaccgacgacgaggacgacgacctggccgacgtggactacgtgcctccggctcctagacgcgctcctagaaggggaggtggcggtgctggagcaactaggggaaccagccagcctgccgctacaagacccgcacctcctggtgcaccgcggagcagcagtagcggtggcgcacctctgcgggcaggcgttggatcaggctccggaggcggaccagctgtggccgccgtggttcctcgggtggccagcctaccacctgcagccggtggaggcagagcgcaagctcgacgggtgggagaggacgcagctgctgccgaaggccggacaccacccgctaggcaaccacgggcagcccaggagcctcccatcgtgatcagcgacagcccgccaccaagtccccggagacctgccggacctggacccctgagcttcgtgagcagcagcagcgcccaggtgtcaagcggtccaggaggcggaggcttaccccagagcagcgggagggctgcaagaccacgcgctgcagtggctccgcgggtgagatctccgccacgtgccgctgcagcccctgtggtgagcgcatccgctgacgctgccggtcctgcacctcctgccgtgcccgttgacgcccatagagctccccggagccggatgacccaggcccagaccgacacccaagctcaaagcctgggtcgtgccggtgcaacagacgccaggggcagtggtggaccaggtgcagagggcgggcctggagttccacggggaaccaacacccctggtgccgctccacacgctgccgaaggggctgctgctggcggaggcagtggaggtggctctccagcccctggactgacccggtacctgcccatcgccggcgtgagcagcgtggtggccctggctccctacgtgaacaagaccgtgaccggcgactgcttacccgtgctggacatggagaccggccacatcggcgcctacgtggtgctggtggaccagaccggcaacgtggccgacctcctgagagccgccgcaccagcttggagccggagaaccctgctgcctgagcacgcccggaactgcgtgcggcctcccgactaccccacaccgcctgccagcgagtggaacagcctgtggatgacgcccgtgggcaacatgctgttcgaccagggcaccctggtgggagccctggacttccacggcctgaggagccggcacccttggagccgggagcaaggcgcaccctaaagctccccgggggcctcggtggcctagcttcttgccccttgggcctccccccagcccctcctccccttcctgcaggagattgagtgtagtggtggtctttgaataaagtctgagtgggcggc

SEQ ID NO: 108 - RNA, 2265 nt, synthetic construct
  source 1..2265: mol_type = other RNA
gaggaaatcgcaaaatttgctcttcgcgttagatttcttttagttttctcgcaactagcaagctttttgttctcgccatgcgggtgctgtacggtggcctgggcgactcaagacctggcctctggggcgcacctgaggcagaggaagcgcgggctcgttttgaggcatctggtgcccctgcccctgtgtgggccccagagctgggagacgccgcccagcagtacgccctgatcacccggctgctgtacacacccgacgccgaggccatgggctggctgcagaaccctcgggtggctccaggggacgtggccctggaccaggcctgcttcagaataagcggggccgctcggaacagcagcagcttcatcagcggcagcgttgctcgagccgtgcctcacctgggctacgcaatggccgccggcagattcggctggggcctggctcacgtcgctgcggcagtggctatgagccggcggtacgacagggcccagaagggcttcctgttgacaagcctgcgccgcgcatacgctccactgttagcccgcgagaacgccgcactgacgggagcccggactcctgacgacggcggagacgccaacagacacgacggggacgacgcaagaggtaaaccgcccctgcccagtgctgcggcctctcccgctgacgagagagccgtgcctgcaggctacggcgcagctggggtgctggcagcactgggcaggctgtcagccgcccctgctagcgctccagctggcgctagggctgaagcaggcagggtggccgtggagtgcttagccgcctgtcggggcatcctggaggccctggccgaaggctttgacggggacctggctgcggtgcctggcttggccggtgccagacctgcagcacctccccgacctggtccagcaggcgcagcagctcctcctcacgctgacgccccacgcctgagagcctggctgcgggagctgcggttcgtgcgggacgccctggtgctgatgcggctgagaggcgacctgcgtgttgctggcggttccgaggcagctgtggccgcagtgagagccgttagcctggtggccggcgcattaggaccagccctgccacgtagcccgagactgctgagcagcgacctgctgttccagaatcagtctttgggtggagggtccggtggaggacctcctccaggtggagcacctgccgccttcggccctttaagggcgtctggccctctgcgtagagcagccgcctggatgcggcaggtgcccgatcccgaggacgtgcgggtggtgatcctgtacagtcccctgcccggcgaagatctcgcagccggaagagccggaggcggaccaccacctgagtggagcgccgaacgaggtgggctgagctgtcttctcgccgcgctgggcaacagactctgtggacccgctacagccgcctgggctggcaattggacaggtgccccggacgtatccgccctgggcgcacaaggcgtgctgctgctgagcacccgggatctggcattcgccggcgccgtggagtttctgggactgctggcaggtgcctgtgaccggcggctgatcgtggtcaacgcagtcagagccgctgactggcctgcggacggacctgtggtgagccggcagcacgcctacctggcctgcgaggtgctgccagccgtgcagtgtgcagttcggtggcctgccgccagagacctgcggcggaccgtgttagccagcggccgggttttcggtcccggagtgttcgctcgggtggaagcagcccacgcacggctgtaccccgacgccccacctctgcggctgtgcagaggagcaaacgtgcggtaccgggtgcggacacggttcggcccagacaccctggtgcccatgtcaccccgggagtaccggagagccgtgttacctgcccttgacggaagggcagccgcttctggggccggtgacgctatggctcctggcgctcccgacttctgcgaggacgaagcccacagccaccgagcctgcgccaggtggggtctcggtgccccactgagacccgtgtacgtggctctgggacgggacgctgtacggggtgggcccgctgaacttagagggccacggcgggagttctgtgcacgggccctgctggagtaaagctccccgggggcctcggtggcctagcttcttgccccttgggcctccccccagcccctcctccccttcctgcaggagattgagtgtagtgacgtggtctttgaataaagtctgagtgggcggc

SEQ ID NO: 109 - RNA, 75 nt, synthetic construct
  source 1..75: mol_type = other RNA
ggaaatcgcaaaatttgctcttcgcgttagatttcttttagttttctcgcaactagcaagctttttgttctcgcc

SEQ ID NO: 110 - RNA, 119 nt, synthetic construct
  source 1..119: mol_type = other RNA
taaagctccccgggggcctcggtggcctagcttcttgccccttgggcctccccccagcccctcctccccttcctgcacccgtacccccgtggtctttgaataaagtctgagtgggcggc

SEQ ID NO: 111 - protein, 904 aa, unidentified
  source 1..904: note = Herpes simplex virus 1
MRQGAPARGRRWFVVWALLGLTLGVLVASAAPSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 112 - protein, 904 aa, unidentified
  source 1..904: note = Herpes simplex virus 2
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 113 - protein, 511 aa, unidentified
  source 1..511: note = Herpes simplex virus 1
MAPGRVGLAVVLWSLLWLGAGVAGGSETASTGPTITAGAVTNASEAPTSGPPGSAASPEVTPTSTPNPNNVTQNKTTPTEPASPPTTPKPTSTPKSPPTSTPDPKPKNNTTPAKSGRPTKPPGPVWCDRRDPLARYGSRVQIRCRFRNSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAWGRMDSPHEYGTWVRVRMFRPPSLTLQPHAVMEGQPFKATCTAAAYYPRNPVEFVWFEDDRQVFNPGQIDTQTHEHPDGFTTVSTVTSEAVGGQVPPRTFTCQMTWHRDSVTFSRRNATGLALVLPRPTITMEFGVRHVACTAGCVPEGVTFTWFLGDDPSPAAKSAVTAQESCDHPGLATVRSTLPISYDYSEYICRLTGYPAGIPVLEHHGSHQPPPRDPTERQVIEAIEWVGIGIGVLAAGVLVVTAIVYVVRTSQSRQRHRR

SEQ ID NO: 114 - protein, 480 aa, unidentified
  source 1..480: note = Herpes simplex virus 2
MALGRVGLAVGLWGLLWVGVVVVLANASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHHGSHQPPPRDPTERQVIRAVEGAGIGVAVLVAVVLAGTAVVYLTHASSVRYRRLR

SEQ ID NO: 115 - protein, 394 aa, unidentified
  source 1..394: note = Herpes simplex virus 1
MGGAAARLGAVILFVVIVGLHGVRSKYALVDASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRHTQKAPKRIRLPHIREDDQPSSHQPLFY

SEQ ID NO: 116 - protein, 393 aa, unidentified
  source 1..393: note = Herpes simplex virus 2
MGRLTSGVGTAALLVVAVGLRVVCAKYALADPSLKMADPNRFRGKNLPVLDRLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 117 - protein, 775 aa, unidentified
  source 1..775: note = Herpes simplex virus 1
MEPRPGASTRRPEGRPQREPAPDVWVFPCDRDLPDSSDSEAETEVGGRGDADHHDDDSASEADSTDTELFETGLLGPQGVDGGAVSGGSPPREEDPGSCGGAPPREDGGSDEGDVCAVCTDEIAPHLRCDTFPCMHRFCIPCMKTWMQLRNTCPLCNAKLVYLIVGVTPSGSFSTIPIVNDPQTRMEAEEAVRAGTAVDFIWTGNQRFAPRYLTLGGHTVRALSPTHPEPTTDEDDDDLDDADYVPPAPRRTPRAPPRRGAAAPPVTGGASHAAPQPAAARTAPPSAPIGPHGSSNTNTTTNSSGGGGSRQSRAAAPRGASGPSGGVGVGVGVVEAEAGRPRGRTGPLVNRPAPLANNRDPIVISDSPPASPHRPPAAPMPGSAPRPGPPASAAASGPARPRAAVAPCVRAPPPGPGPRAPAPGAEPAARPADARRVPQSHSSLAQAANQEQSLCRARATVARGSGGPGVEGGHGPSRGAAPSGAAPLPSAASVEQEAAVRPRKRRGSGQENPSPQSTRPPLAPAGAKRAATHPPSDSGPGGRGQGGPGTPLTSSAASASSSSASSSSAPTPAGAASSAAGAASSSASASSGGAVGALGGRQEETSLGPRAASGPRGPRKCARKTRHAETSGAVPAGGLTRYLPISGVSSVVALSPYVNKTITGDCLPILDMETGNIGAYVVLVDQTGNMATRLRAAVPGWSRRTLLPETAGNHVMPPEYPTAPASEWNSLWMTPVGNMLFDQGTLVGALDFRSLRSRHPWSGEQGASTRDEGKQ

SEQ ID NO: 118 - protein, 825 aa, unidentified
  source 1..825: note = Herpes simplex virus 2
MEPRPGTSSRADPGPERPPRQTPGTQPAAPHAWGMLNDMQWLASSDSEEETEVGISDDDLHRDSTSEAGSTDTEMFEAGLMDAATPPARPPAERQGSPTPADAQGSCGGGPVGEEEAEAGGGGDVCAVCTDEIAPPLRCQSFPCLHPFCIPCMKTWIPLRNTCPLCNTPVAYLIVGVTASGSFSTIPIVNDPRTRVEAEAAVRAGTAVDFIWTGNPRTAPRSLSLGGHTVRALSPTPPWPGTDDEDDDLADVDYVPPAPRRAPRRGGGGAGATRGTSQPAATRPAPPGAPRSSSSGGAPLRAGVGSGSGGGPAVAAVVPRVASLPPAAGGGRAQARRVGEDAAAAEGRTPPARQPRAAQEPPIVISDSPPPSPRRPAGPGPLSFVSSSSAQVSSGPGGGGLPQSSGRAARPRAAVAPRVRSPPRAAAAPVVSASADAAGPAPPAVPVDAHRAPRSRMTQAQTDTQAQSLGRAGATDARGSGGPGAEGGPGVPRGTNTPGAAPHAAEGAAARPRKRRGSDSGPAASSSASSSAAPRSPLAPQGVGAKRAAPRRAPDSDSGDRGHGPLAPASAGAAPPSASPSSQAAVAAASSSSASSSSASSSSASSSSASSSSASSSSASSSSASSSAGGAGGSVASASGAGERRETSLGPRAAAPRGPRKCARKTRHAEGGPEPGARDPAPGLTRYLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGALDFHGLRSRHPWSREQGAPAPAGDAPAGHGE

SEQ ID NO: 119 - protein, 1298 aa, unidentified
  source 1..1298: note = Herpes simplex virus 1
MASENKQRPGSPGPTDGPPPTPSPDRDERGALGWGAETEEGGDDPDHDPDHPHDLDDARRDGRAPAAGTDAGEDAGDAVSPRQLALLASMVEEAVRTIPTPDPAASPPRTPAFRADDDDGDEYDDAADAAGDRAPARGREREAPLRGAYPDPTDRLSPRPPAQPPRRRRHGRWRPSASSTSSDSGSSSSSSASSSSSSSDEDEDDDGNDAADHAREARAVGRGPSSAAPAAPGRTPPPPGPPPLSEAAPKPRAAARTPAASAGRIERRRARAAVAGRDATGRFTAGQPRRVELDADATSGAFYARYRDGYVSGEPWPGAGPPPPGRVLYGGLGDSRPGLWGAPEAEEARRRFEASGAPAAVWAPELGDAAQQYALITRLLYTPDAEAMGWLQNPRVVPGDVALDQACFRISGAARNSSSFITGSVARAVPHLGYAMAAGRFGWGLAHAAAAVAMSRRYDRAQKGFLLTSLRRAYAPLLARENAALTGAAGSPGAGADDEGVAAVAAAAPGERAVPAGYGAAGILAALGRLSAAPASPAGGDDPDAARHADADDDAGRRAQAGRVAVECLAACRGILEALAEGFDGDLAAVPGLAGARPASPPRPEGPAGPASPPPPHADAPRLRAWLRELRFVRDALVLMRLRGDLRVAGGSEAAVAAVRAVSLVAGALGPALPRDPRLPSSAAAAAADLLFDNQSLRPLLAAAASAPDAADALAAAAASAAPREGRKRKSPGPARPPGGGGPRPPKTKKSGADAPGSDARAPLPAPAPPSTPPGPEPAPAQPAAPRAAAAQARPRPVAVSRRPAEGPDPLGGWRRQPPGPSHTAAPAAAALEAYCSPRAVAELTDHPLFPVPWRPALMFDPRALASIAARCAGPAPAAQAACGGGDDDDNPHPHGAAGGRLFGPLRASGPLRRMAAWMRQIPDPEDVRVVVLYSPLPGEDLAGGGASGGPPEWSAERGGLSCLLAALANRLCGPDTAAWAGNWTGAPDVSALGAQGVLLLSTRDLAFAGAVEFLGLLASAGDRRLIVVNTVRACDWPADGPAVSRQHAYLACELLPAVQCAVRWPAARDLRRTVLASGRVFGPGVFARVEAAHARLYPDAPPLRLCRGGNVRYRVRTRFGPDTPVPMSPREYRRAVLPALDGRAAASGTTDAMAPGAPDFCEEEAHSHAACARWGLGAPLRPVYVALGREAVRAGPARWRGPRRDFCARALLEPDDDAPPLVLRGDDDGPGALPPAPPGIRWASATGRSGTVLAAAGAVEVLGAEAGLATPPRREVVDWEGAWDEDDGGAFEGDGVL

SEQ ID NO: 120 - protein, 1318 aa, unidentified
  source 1..1318: note = Herpes simplex virus 2
MSAEQRKKKKTTTTTQGRGAEVAMADEDGGRLRAAAETTGGPGSPDPADGPPPTPNPDRRPAARPGFGWHGGPEENEDEADDAAADADADEAAPASGEAVDEPAADGVVSPRQLALLASMVDEAVRTIPSPPPERDGAQEEAARSPSPPRTPSMRADYGEENDDDDDDDDDDDRDAGRWVRGPETTSAVRGAYPDPMASLSPRPPAPRRHHHHHHHRRRRAPRRRSAASDSSKSGSSSSASSASSSASSSSSASASSSDDDDDDDAARAPASAADHAAGGTLGADDEEAGVPARAPGAAPRPSPPRAEPAPARTPAATAGRLERRRARAAVAGRDATGRFTAGRPRRVELDADAASGAFYARYRDGYVSGEPWPGAGPPPPGRVLYGGLGDSRPGLWGAPEAEEARARFEASGAPAPVWAPELGDAAQQYALITRLLYTPDAEAMGWLQNPRVAPGDVALDQACFRISGAARNSSSFISGSVARAVPHLGYAMAAGRFGWGLAHVAAAVAMSRRYDRAQKGFLLTSLRRAYAPLLARENAALTGARTPDDGGDANRHDGDDARGKPAAAAAPLPSAAASPADERAVPAGYGAAGVLAALGRLSAAPASAPAGADDDDDDDGAGGGGGGRRAEAGRVAVECLAACRGILEALAEGFDGDLAAVPGLAGARPAAPPRPGPAGAAAPPHADAPRLRAWLRELRFVRDALVLMRLRGDLRVAGGSEAAVAAVRAVSLVAGALGPALPRSPRLLSSAAAAAADLLFQNQSLRPLLADTVAAADSLAAPASAPREARKRKSPAPARAPPGGAPRPPKKSRADAPRPAAAPPAGAAPPAPPTPPPRPPRPAALTRRPAEGPDPQGGWRRQPPGPSHTPAPSAAALEAYCAPRAVAELTDHPLFPAPWRPALMFDPRALASLAARCAAPPPGGAPAAFGPLRASGPLRRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGAGDAMAPGAPDFCEDEAHSHRACARWGLGAPLRPVYVALGRDAVRGGPAELRGPRREFCARALLEPDGDAPPLVLRDDADAGPPPQIRWASAAGRAGTVLAAAGGGVEVVGTAAGLATPPRREPVDMDAELEDDDDGLFGE

SEQ ID NO: 121 - protein, 32 aa, synthetic construct
GSGGSGSGGSSGGGSGSGGSGGSGSGGRRRRR